(19) United States
(12) Patent Application Publication
(10) Pub. No.: US 2025/0213698 A1
(43) Pub. Date: Jul. 3, 2025

(54) BICYCLIC HETEROCYCLES AND THEIR LIGANDS FOR TARGETED DELIVERY OF THERAPEUTIC AGENTS

(71) Applicants: HANSOH BIO LLC, Rockville, MD (US); SHANGHAI HANSOH BIOMEDICAL CO., LTD., Shanghai (CN); JIANGSU HANSOH PHARMACEUTICAL GROUP CO., LTD., Lianyungang (CN)

(72) Inventors: Xiaoyang Guan, Rockville, MD (US); Hugh Y. Zhu, Rockville, MD (US); Hong Zong, Rockville, MD (US)

(21) Appl. No.: 18/850,048

(22) PCT Filed: Mar. 24, 2023
(86) PCT No.: PCT/CN2023/083768

§ 371 Date: Sep. 23, 2024

Related U.S. Application Data
(60) Provisional application No. 63/269,903, filed on Mar. 25, 2022.
(60) Provisional application No. 63/382,171, filed on Nov. 3, 2022.

(51) Int. Cl. A61K 47/54; C07C 215/44; C07D 203/26; C07D 205/12; C07D 209/44; C07D 209/52; C07D 221/20; C07D 257/02; C07D 471/08; C07D 487/08; C07H 15/04; C07H 15/26; C07H 21/00; C12N 15/113

(52) U.S. Cl. CPC A61K 47/549; C07C 215/44; C07D 203/26; C07D 205/12; C07D 209/44; C07D 209/52; C07D 221/20; C07D 257/02; C07D 471/08; C07D 487/08; C07H 15/04; C07H 15/26; C07H 21/00; C12N 15/1137; C07C 2602/22; C12N 2310/14; C12N 2310/351

(57) ABSTRACT

the present invention provides novel bicyclic heterocycles and their targeting ligands which can conjugate to a therapeutic agent, for use as medicament. The preparation method thereof, pharmaceutical compositions comprising the therapeutic compounds, and the pharmaceutical uses are disclosed.

### FIELD OF THE INVENTION

The present invention belongs to the field of medicine, and relates to bicyclic heterocycles, GalNAc-conjugation, oligonucleotide therapeutics, preparation methods thereof, pharmaceutical compositions comprising the compounds, and medical uses thereof.

### BACKGROUND OF THE INVENTION

Targeted delivery of oligonucleotides to liver hepatocytes using N-acetylgalactosamine (GalNAc) conjugates that bind to the asialoglycoprotein receptor (ASGPR) has become a breakthrough approach in the therapeutic oligonucleotide field. ASGPR is the ideal target for delivery of therapeutic oligonucleotides to the liver since it combines tissue specificity, high expression levels (500,000 copies/cell) and rapid internalization and turnover (15 min). GalNAC-conjugation leads to several FDA approved siRNA drugs and many clinical candidates against a diverse range of targets in liver hepatocytes. Beyond oligonucleotides, GalNAc has also been conjugated to small molecules, peptides, proteins and LNPs for targeting hepatocytes. It has also been well documented that the dramatic hierarchy of the inhibitory ability of different valences of oligosaccharides to inhibit the binding of labeled ligand to the mammalian hepatic lectin on hepatocytes is as follows: tetraantennary>triantennary>>biantennary>>monoantennary. With the advantages of GalNAc-conjugation as powerful delivery platform, diversified GalNAc-conjugation structures are important to improve the performance of different modalities.

Other GalNAc conjugation moieties have been disclosed previously. The GalNAc was linked with oligonucleotides through different linkers. Among them, triantennary GalNAc was formed by the usage of tetra-functional groups and tri-functional groups.

Tetra-functional groups tether three GalNAc through different long linkers. Tri-functional groups connect the GalNAc, oligonucleotide and solid support (during the synthesis). Tris, tri-acid, hydroxyprolinol, glycerol and other derivants have been widely used as tetra-functional groups and tri-functional groups. Despite the advances in application of oligonucleotides and oligonucleotide analogs as therapeutics, the need exists for oligonucleotides having improved pharmacological properties.

### SUMMARY OF THE INVENTION

The present invention disclosed a series of tetra-functional groups and tri-functional groups including bicyclic heterocycles and multi-cyclohexane structures, which could link the GalNAc and oligonucleotides and other therapeutics with unique shapes and properties. In one aspect, provides a targeting ligand having the structure shown in formula (I-a):

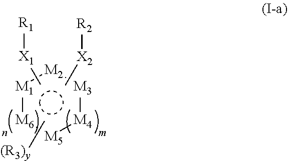

wherein:
X₁ and X₂ are each independently selected from the group of NRₐ(CH₂)ᵣ—, —O(CH₂)ᵣ— and —S(CH₂)ᵣ—, R₁ and R₂ are linked to the N, O or S;
M₁ is C, CH or N;
M₂ is absent, O, C(O) or CH₂;
M₃ is absent, CH, C, N, O, S;
each of M₄ and M₆ is independently selected from C(O) and CH₂;
M₅ is CH, CH₂ or NH;
Rₐ is selected from the group of hydrogen, deuterium, halogen, amino, cyano, hydroxy, alkyl, alkoxy, alkylthio and haloalkyl;
R₁ is hydrogen or a protecting group;
R₂ is hydrogen or a solid support, optionally linked via a linker group, or a phosphoramidite;
R₃ is independently selected from hydrogen, amino, —(CH₂)ᵣCOOH, alkylamino or hydroxyalkyl;
or, two of R₃ together with the C atom to which they are attached form heterocyclyl containing N atom; and the heterocyclyl containing N atom has the structure shown in formula (II):

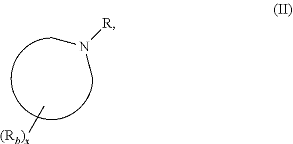

R is a carbohydrate or a derivative thereof having hydroxyl protecting group, linked via a linker group;
R_{b} is independently selected from the group of hydrogen, oxo and alkyl;
m is 0, 1, 2, 3; 4, 5 or 6;
n is 0, 1, 2, 3; 4, 5 or 6;
x is 1, 2, 3 or 4;
y is 1, 2, 3 or 4; and
each of r is independently selected from 0, 1, 2 and 3.

The present invention disclosed a series of tetra-functional groups and tri-functional groups including bicyclic heterocycles and multi-cyclohexane structures, which could link the GalNAc and oligonucleotides and other therapeutics with unique shapes and properties. In one aspect, provides a targeting ligand having the structure shown in formula (I):

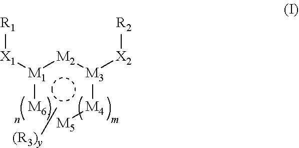

wherein:
X₁ and X₂ are each independently selected from the group of NRₐ(CH₂)ᵣ—, —O(CH₂)ᵣ— and —S(CH₂)ᵣ—, R₁ and R₂ are linked to the N, O or S;
M₁ is C, CH or N;
M₂ is absent, O, C(O) or CH₂;
M₃ is absent, CH, C, N, O, S;
each of M₄ and M₆ is independently selected from C(O) and CH₂; M₅ is CH, CH₂ or NH;
Rₐ is selected from the group of hydrogen, deuterium, halogen, amino, cyano, hydroxy, alkyl, alkoxy, alkylthio and haloalkyl;
R₁ is hydrogen or a protecting group;
R₂ is hydrogen or a solid support, optionally linked via a linker group, or a phosphoramidite;
R₃ is independently selected from hydrogen, amino, —(CH₂)ᵣCOOH, alkylamino or hydroxyalkyl;
or, two of R₃ together with the C atom to which they are attached form heterocyclyl containing N atom; and the heterocyclyl containing N atom has the structure shown in formula (II):

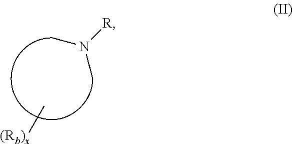

R is a carbohydrate or a derivative thereof having hydroxyl protecting group, linked via a linker group;
R_{b} is independently selected from the group of hydrogen, oxo and alkyl;
m is 0, 1, 2, 3; 4, 5 or 6;
n is 0, 1, 2, 3; 4, 5 or 6;
x is 1, 2, 3 or 4;
y is 1, 2, 3 or 4; and
each of r is independently selected from 0, 1, 2 and 3.

In an embodiment, the targeting ligand having the structure shown in formula (III) or (III-a):

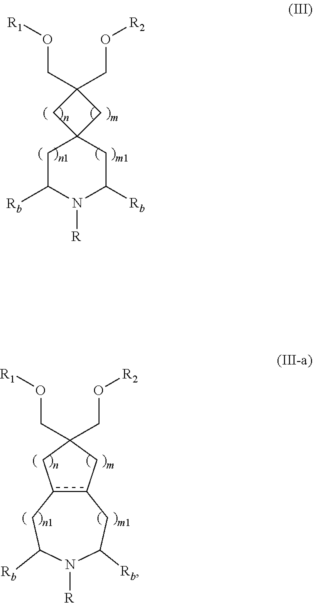

[symbol] is a single or double bond;
R_{b} is independently selected from the group of hydrogen and oxo;
each of m and n is independently selected from 1, 2, 3; 4 and 5
each of m1 and n1 is each independently selected from 0, 1, 2, 3 and 4.

In an embodiment, the targeting ligand having the structure shown in formula (III-b), (III-c) or (III-d):

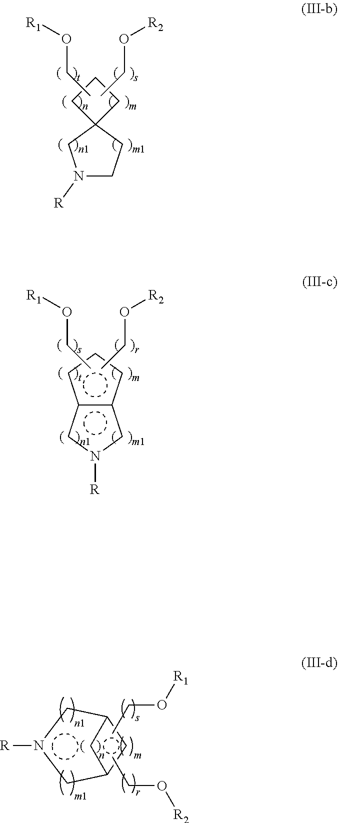

m is independently selected from 0, 1, 2, 3; 4 and 5;
n is independently selected from 1, 2, 3; 4 and 5;
each of r, s and t is independently selected from 0, 1 and 2;
each of m1 and n1 is each independently selected from 0, 1, 2, 3 and 4.

In a preferred embodiment, R is linked via a linker group, having the structure of -linker-R₉, wherein, R₉ is a carbohydrate.

In a more preferred embodiment, R₉ is selected from the group consisting of galactose, galactosamine, N-acetylgalactosamine (GalNAc), D-galactosaminitol, mannose, mannosamine, mannose-6-phosphate, glucose, glucosamine, N-acetyl-glucosamine (GluNAc), glucose-6-phosphate, glucosaminitol, glucose glyceraldehyde, fucose, fucosamine, fuculose, lactose, allose, altrose, arabinose, cladinose, erythrose, erythrulose, fructose, D-fucitol, L-fucitol, L-glycero-D-mannos-heptose, glycerol, glycerone, gulose, idose, lyxose, psicose, quinovose, quinovosamine, rhamnose, rhamnitol, rhamnosamine, ribose, ribulose, sedoheptulose, sorbose, tagatose, talose, tartaric acid, threose, xylose, and xylulose.

In some embodiments, R₉ is N-acetylgalactosamine.

In an embodiment, R is linked via a linker group, having the structure shown in formula (IV), (IV-a), (IV-b) or (IV-c):

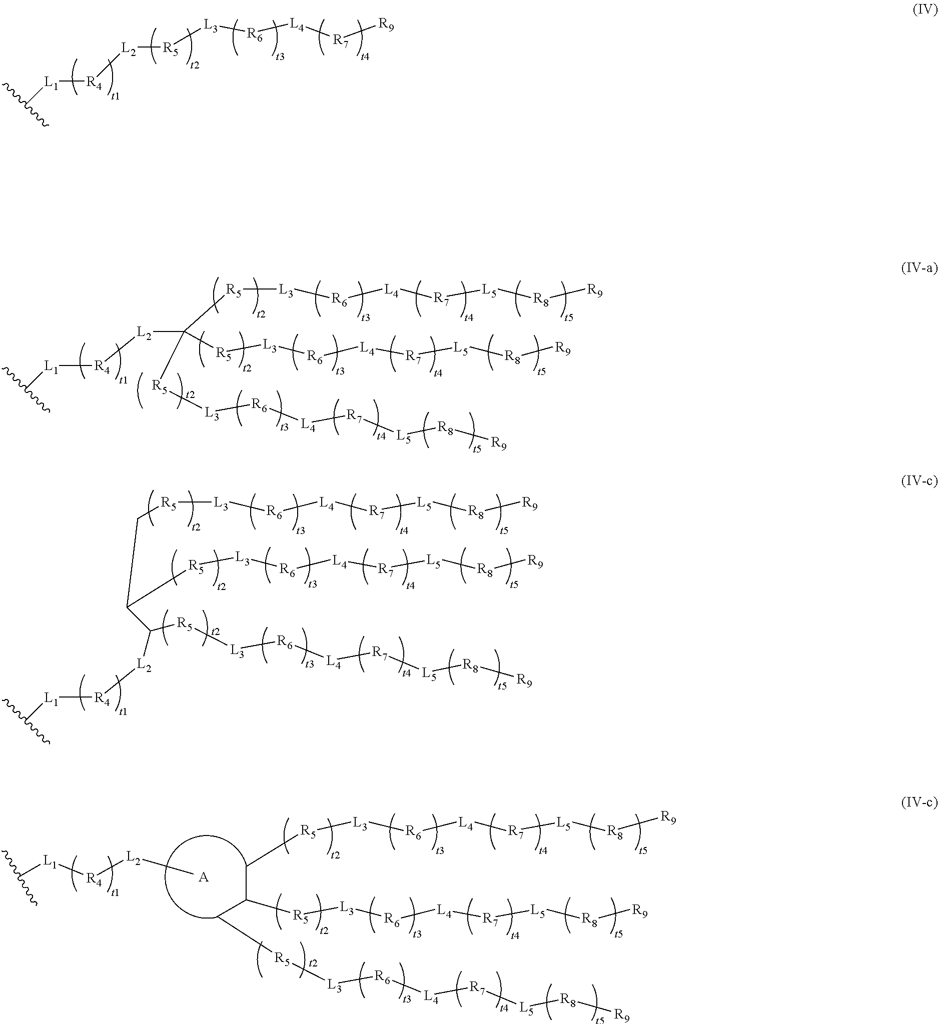

A is C₃₋₁₀ cycloalkyl or 4-12 membered heterocyclyl;
each of L₁, L₂, L₃, L₄ and L₅ is independently selected from the group of substituents consisting of absent, O, S, S—S, NH, CO, CONH, NHCO and 4-10 membered heterocyclyl;
each of R₄, R₆, R₇ and R₈ is independently selected from the group of substituents consisting of CH₂, OCH₂CH₂ and CH₂CH₂O;
R₅ is independently selected from the group of substituents consisting of absent, CH₂, OCH₂, CH₂O, OCH₂CH₂, CH₂CH₂O, NHCH₂, CH₂NH, NHCH₂CH₂, CH₂CH₂NH, C₀₋₆ alkyl (C₃₋₈ cycloalkyl) and C₀₋₆ alkyl (4-10 membered heterocyclyl);
R₉ is a carbohydrate;
each of t1, t2, t3, t4 and t5 is independently selected from 0, 1, 2, 3, 4, 5 and 6.

In an embodiment, R having the structure shown in formula (IV-1), (IV-a-1), (IV-b-1) or (IV-c-1):

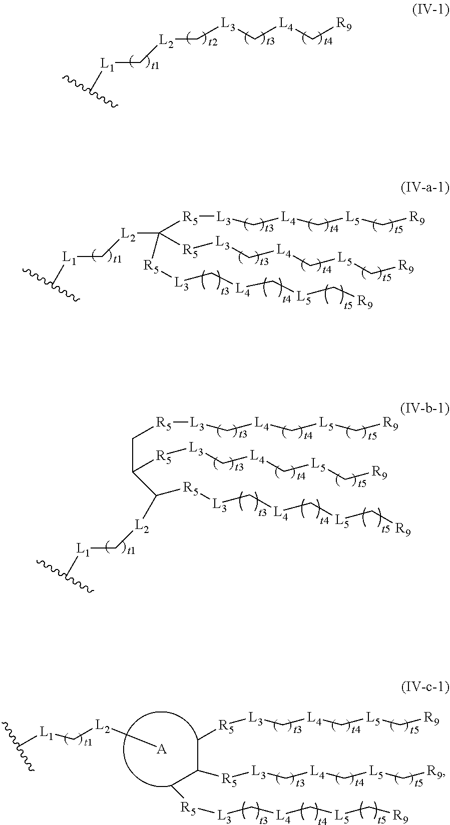

wherein, A is

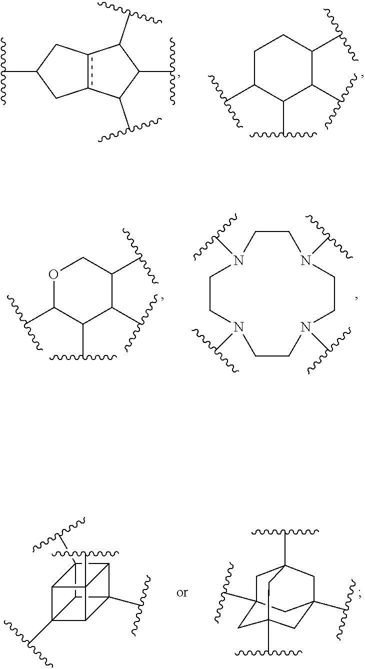

each of L₁, L₃, L₄ and L₅ is independently selected from the group of substituents consisting of absent, O, S, S—S, NH, CO, CONH and NHCO;
L₂ is absent, O, S, S—S, NH, CO, CONH, NHCO and

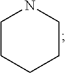

R₅ is independently selected from the group of substituents consisting of absent, CH₂, CH₂O, CH₂NH, C₃₋₆ cycloalkyl, C₃₋₆ CycloalkylC₁₋₃ alkyl, 4-8 membered heterocyclyl C₁₋₃ alkyl containing 1, 2 or 3 of ring heteroatoms selected from N or O and 4-8 membered heterocyclyl containing 1, 2 or 3 of heteroatoms selected from N or O. In some embodiments, R having the structure shown in formula (IV-d), (IV-e), (IV-f), (IV-d-a), (IV-e-a) or (IV-f-a):

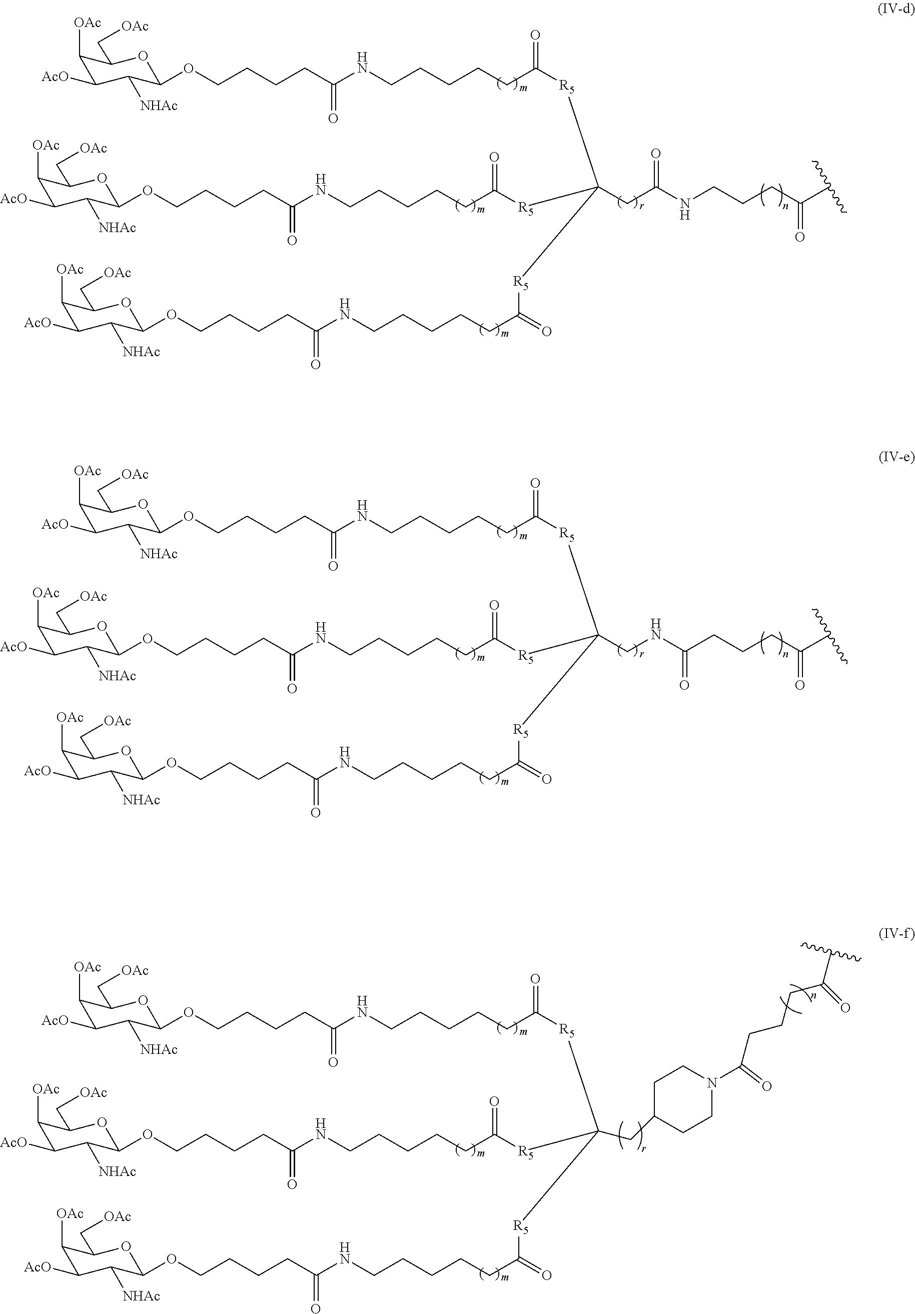

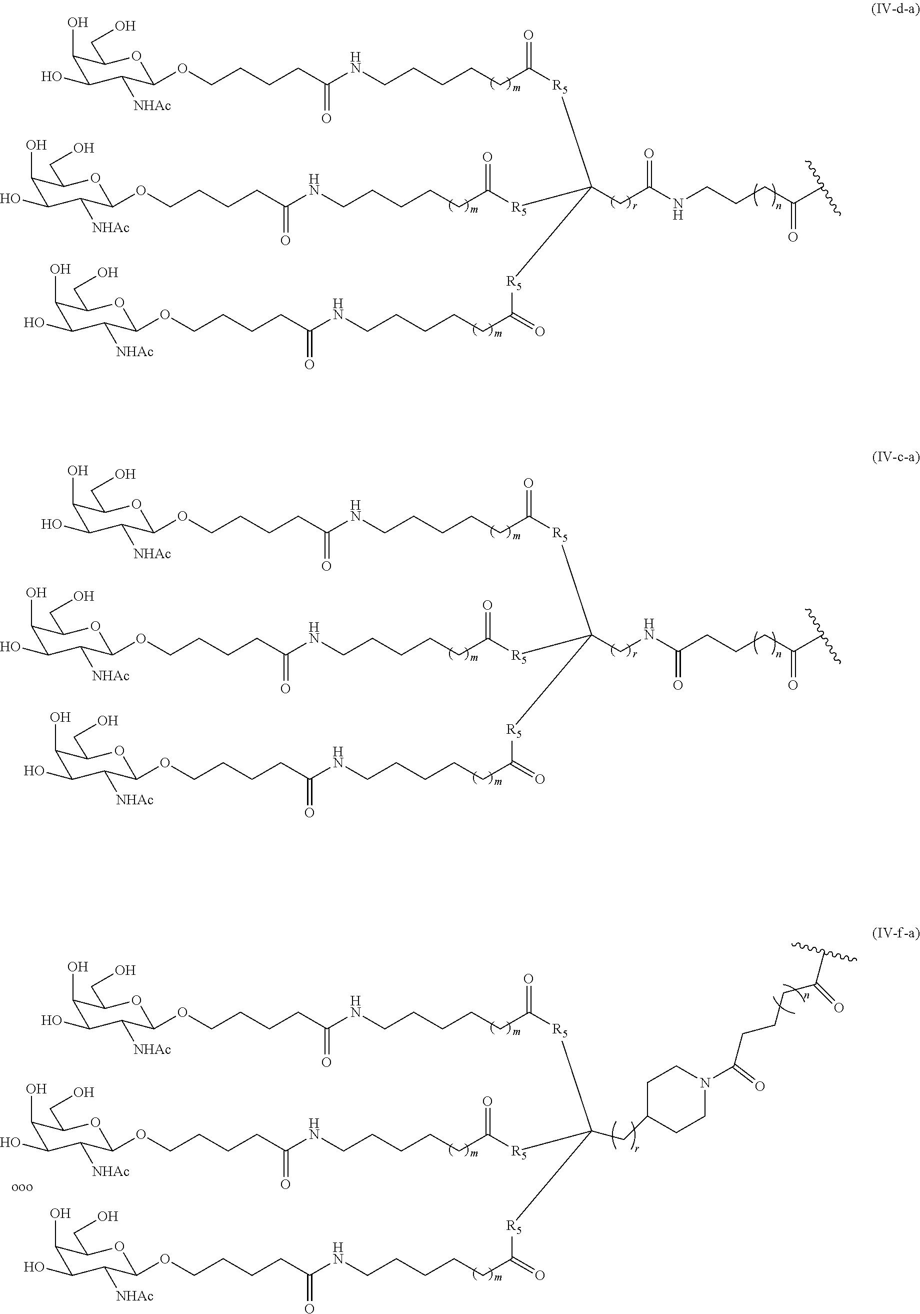

wherein,
R₅ is independently selected from the group of substituents consisting of OCH₂, NHCH₂ and 4-6 membered heterocyclyl containing 1 or 2 of heteroatoms selected from N or O;
m is independently selected from 0, 1, 2, 3, 4, 5 and 6;
n is 1, 2, 3, 4, 5, 6, 7, 8, 9 or 10; and
r is 0, 1, 2, 3, 4 or 5.

In some embodiments, R₉ is N-acetylgalactosamine.

In some embodiments, R₅ is independently selected from —OCH₂—, —NHCH₂—,

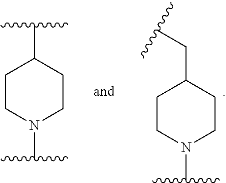 [SMILES: *C1CCN(*)CC1 ; *CC1CCN(*)CC1]

In some embodiments, the R is:

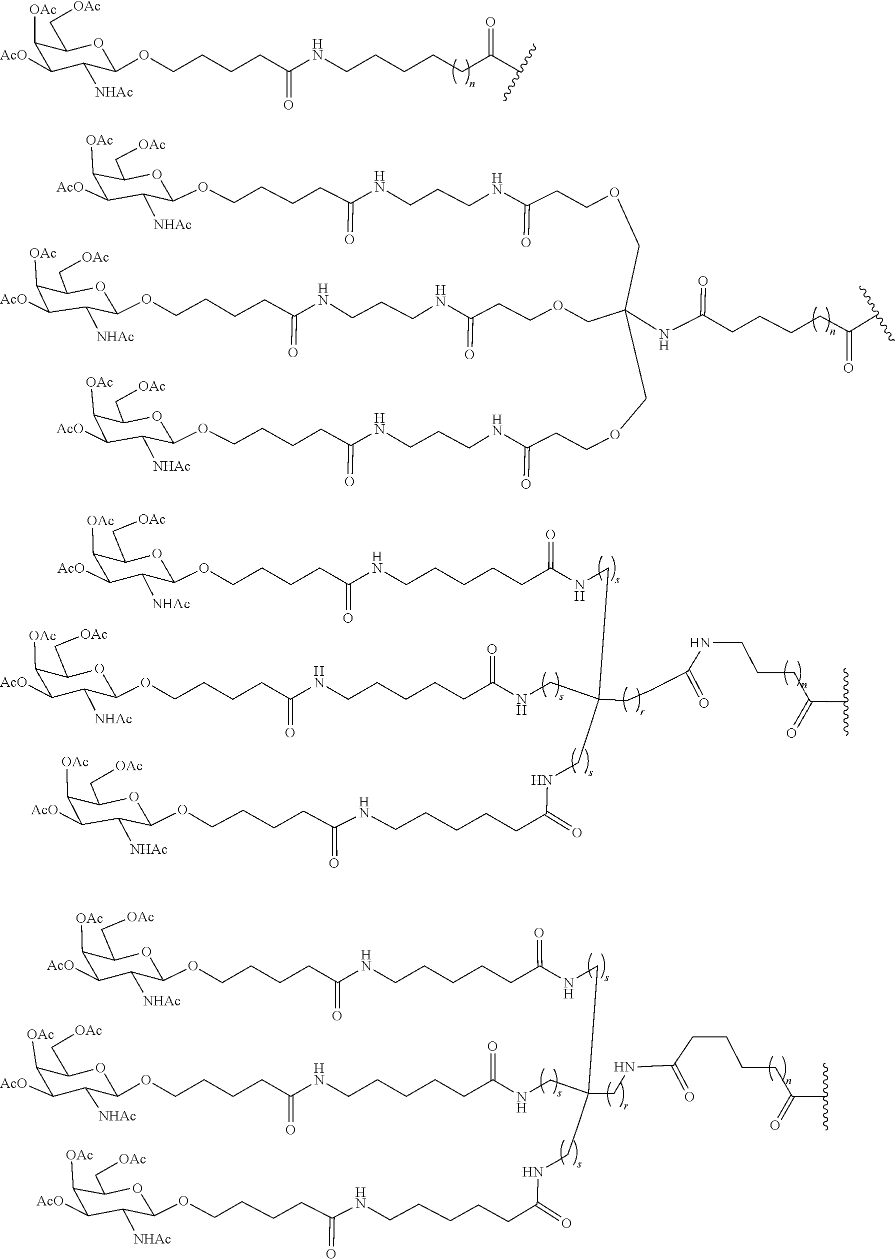

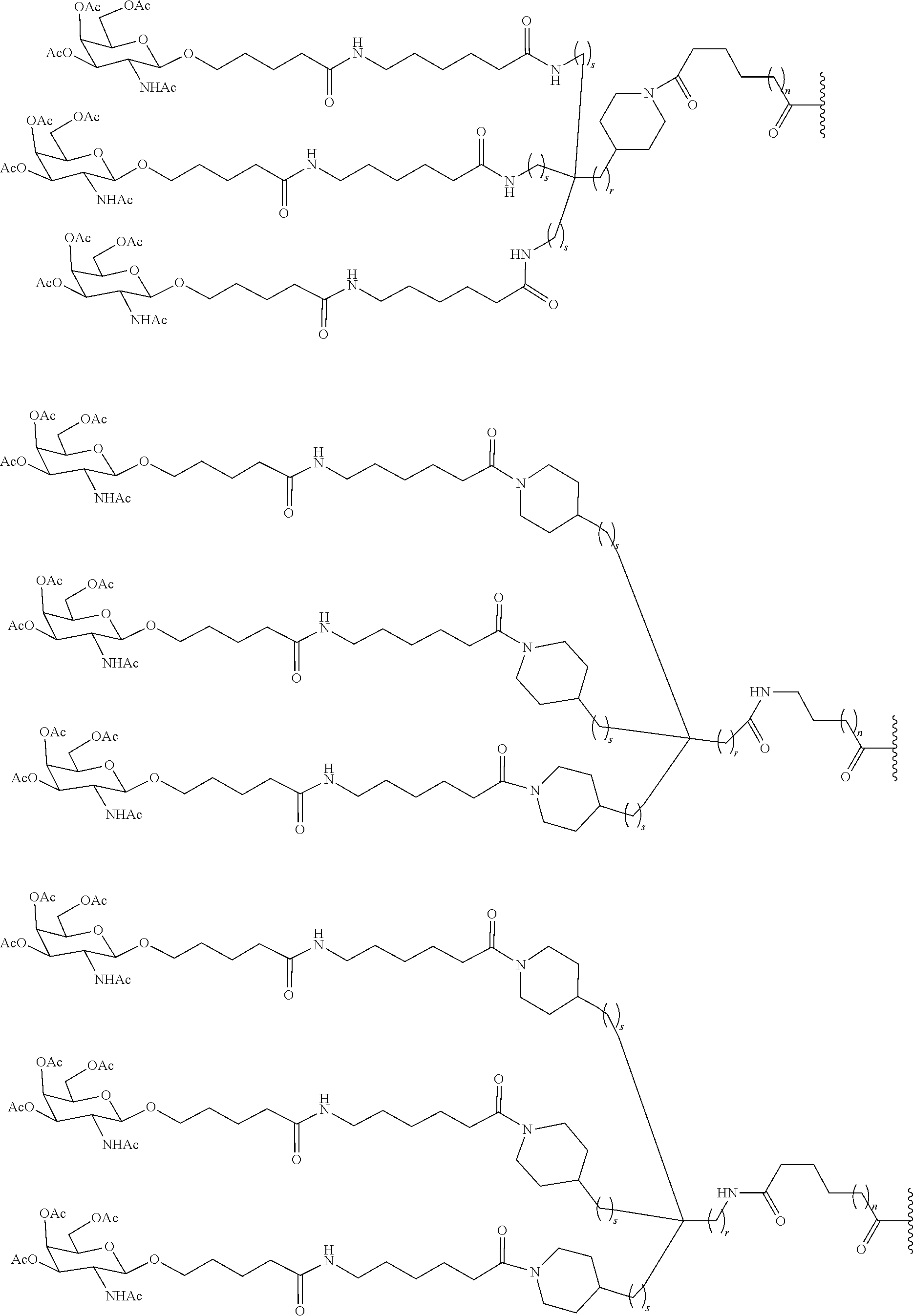

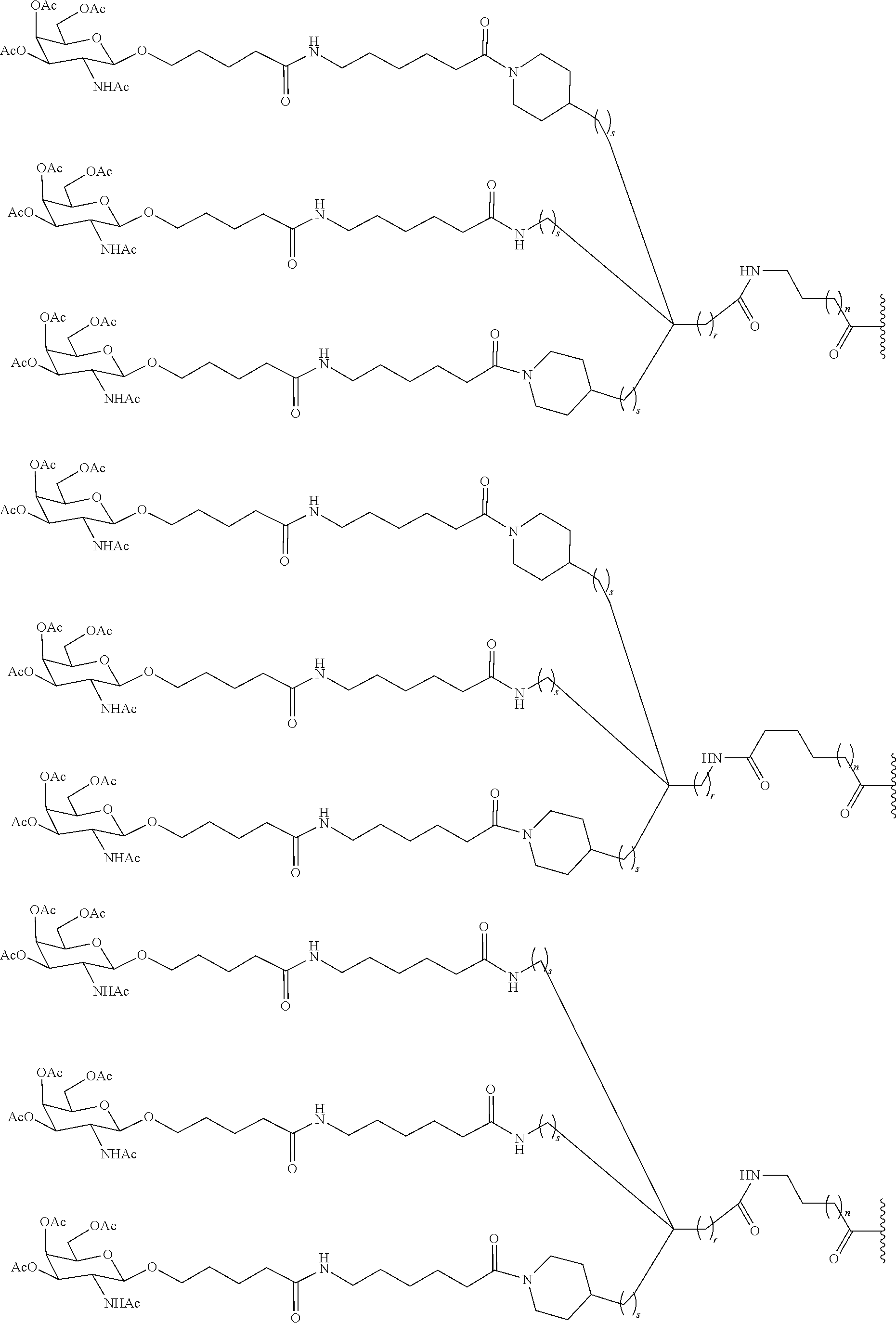

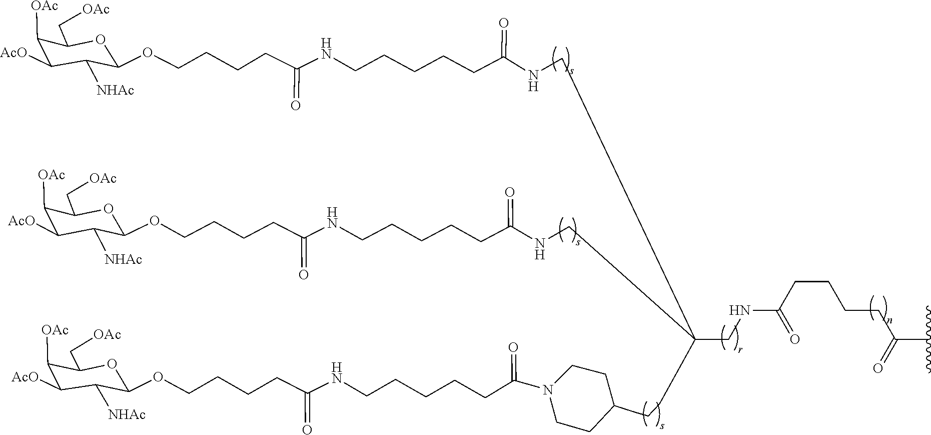

s is independently selected from 0, 1, 2 and 3;
r is independently selected from 0, 1, 2, 3, 4 and 5;
n is independently selected from 1, 2, 3, 4, 5, 6, 7, 8, 9 and 10.

In some embodiments, the R is:

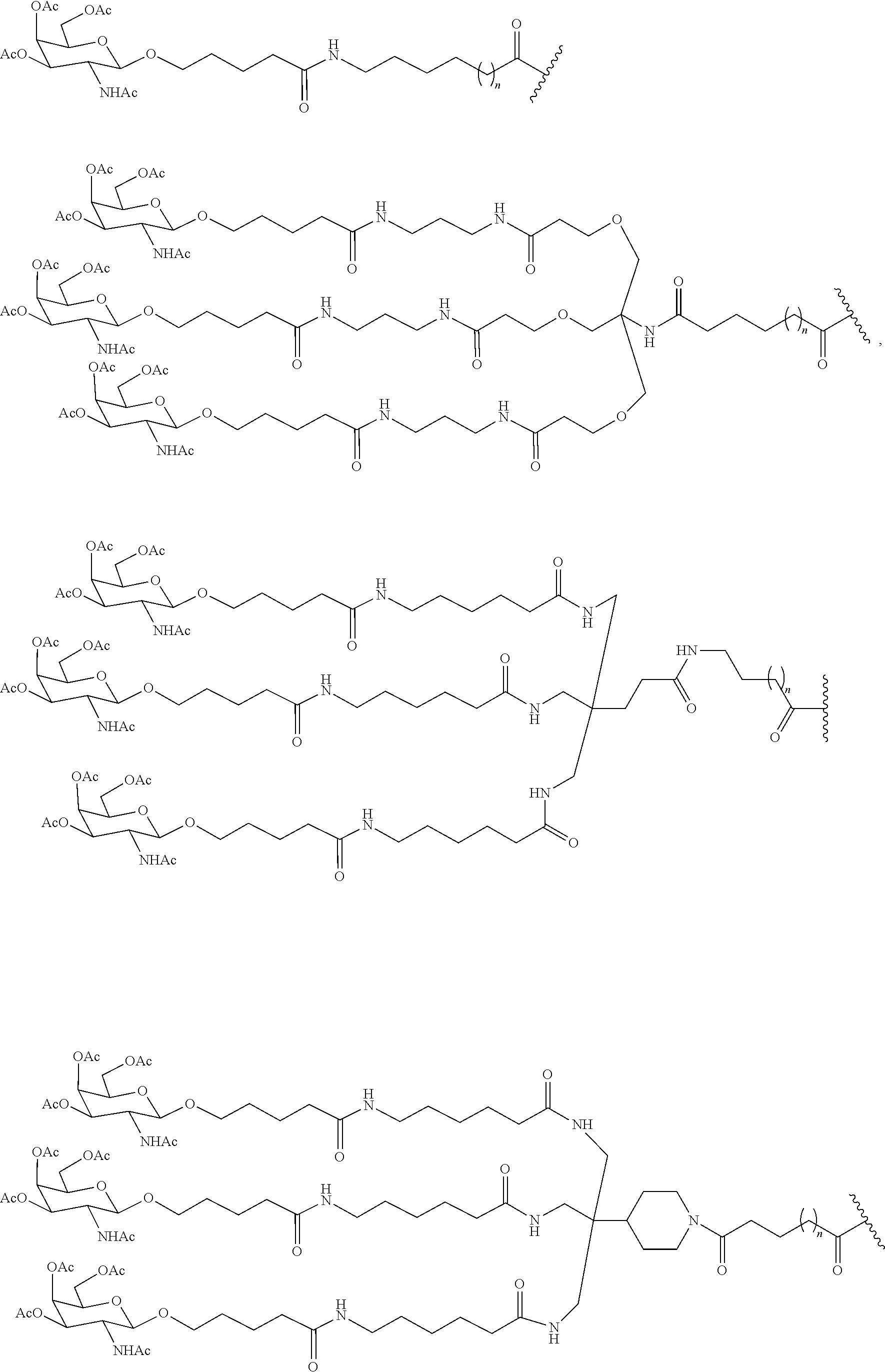

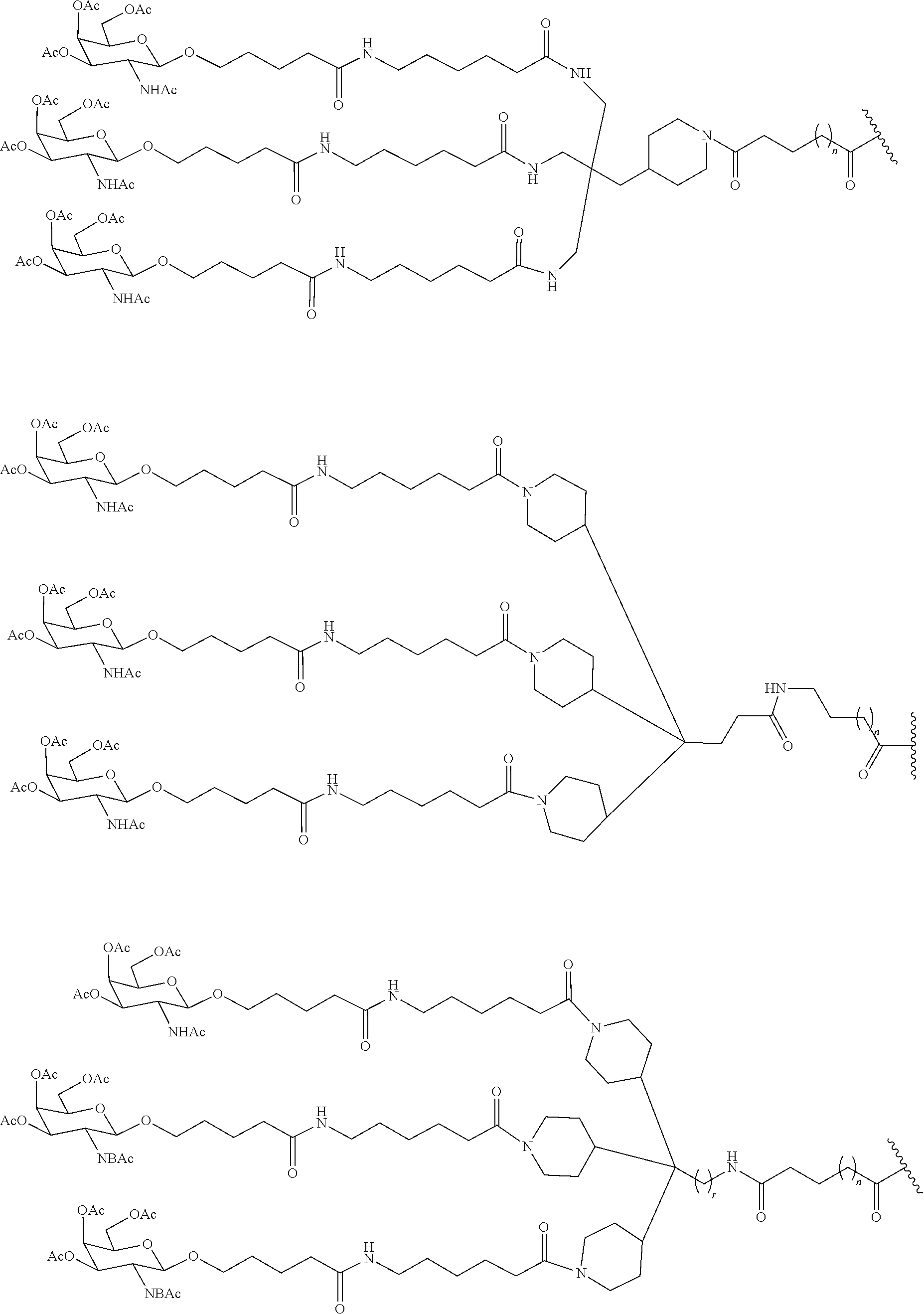

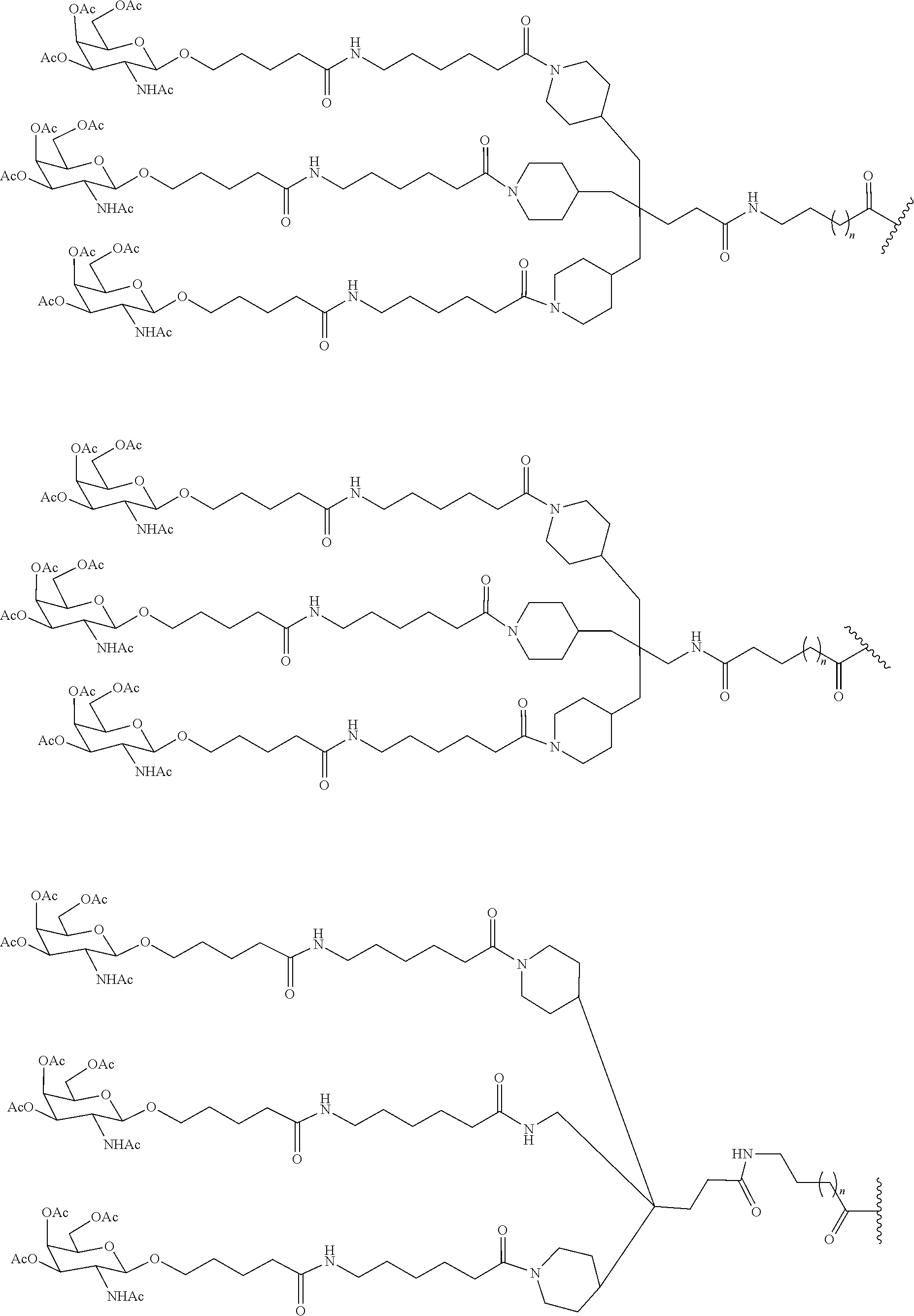

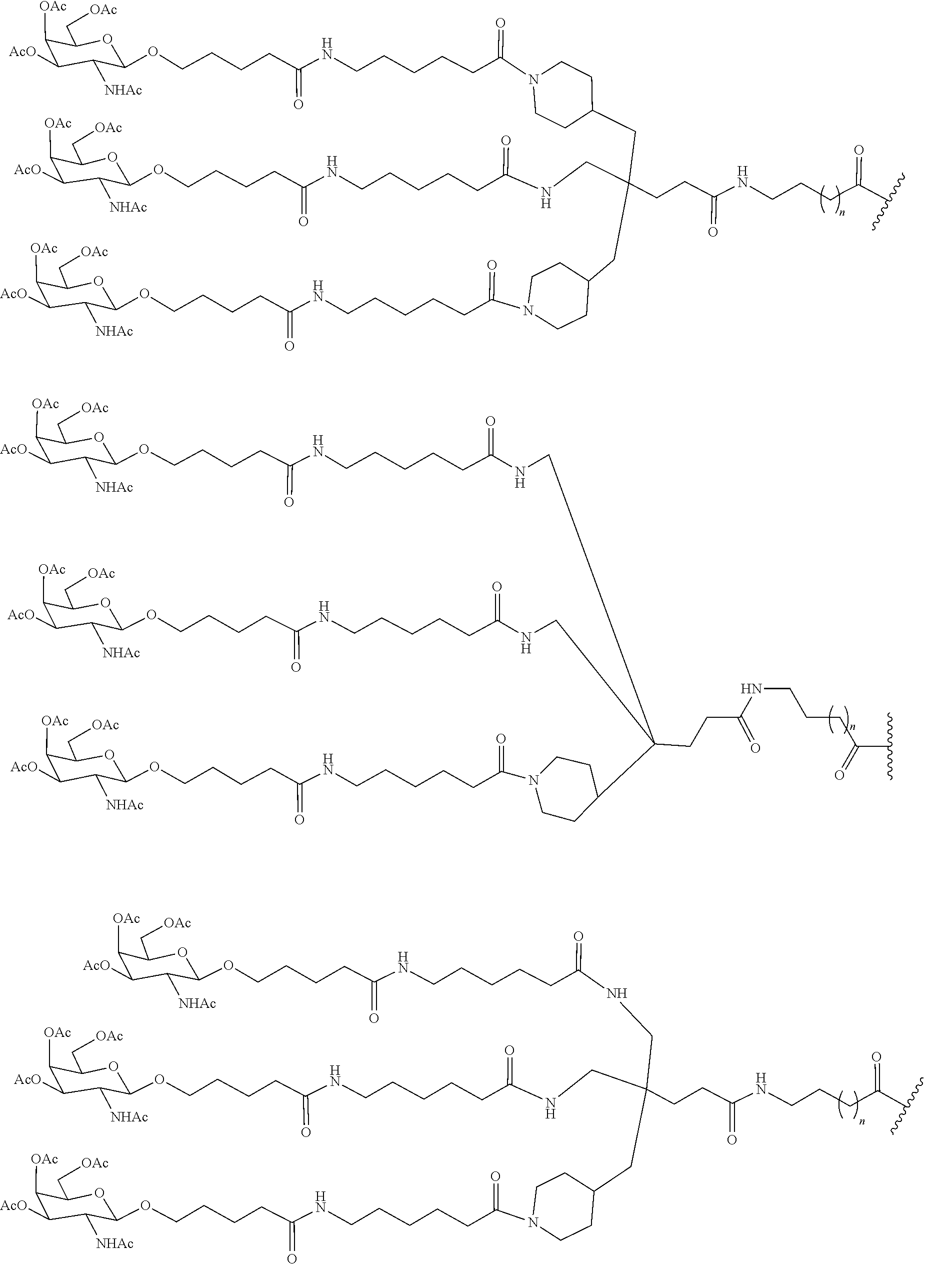

r is independently selected from 0, 1, 2, 3, 4 and 5;
n is independently selected from 1, 2, 3, 4, 5, 6, 7, 8, 9 and 10.

In some embodiments, n is 7 or 8.

In some embodiments, the R is:

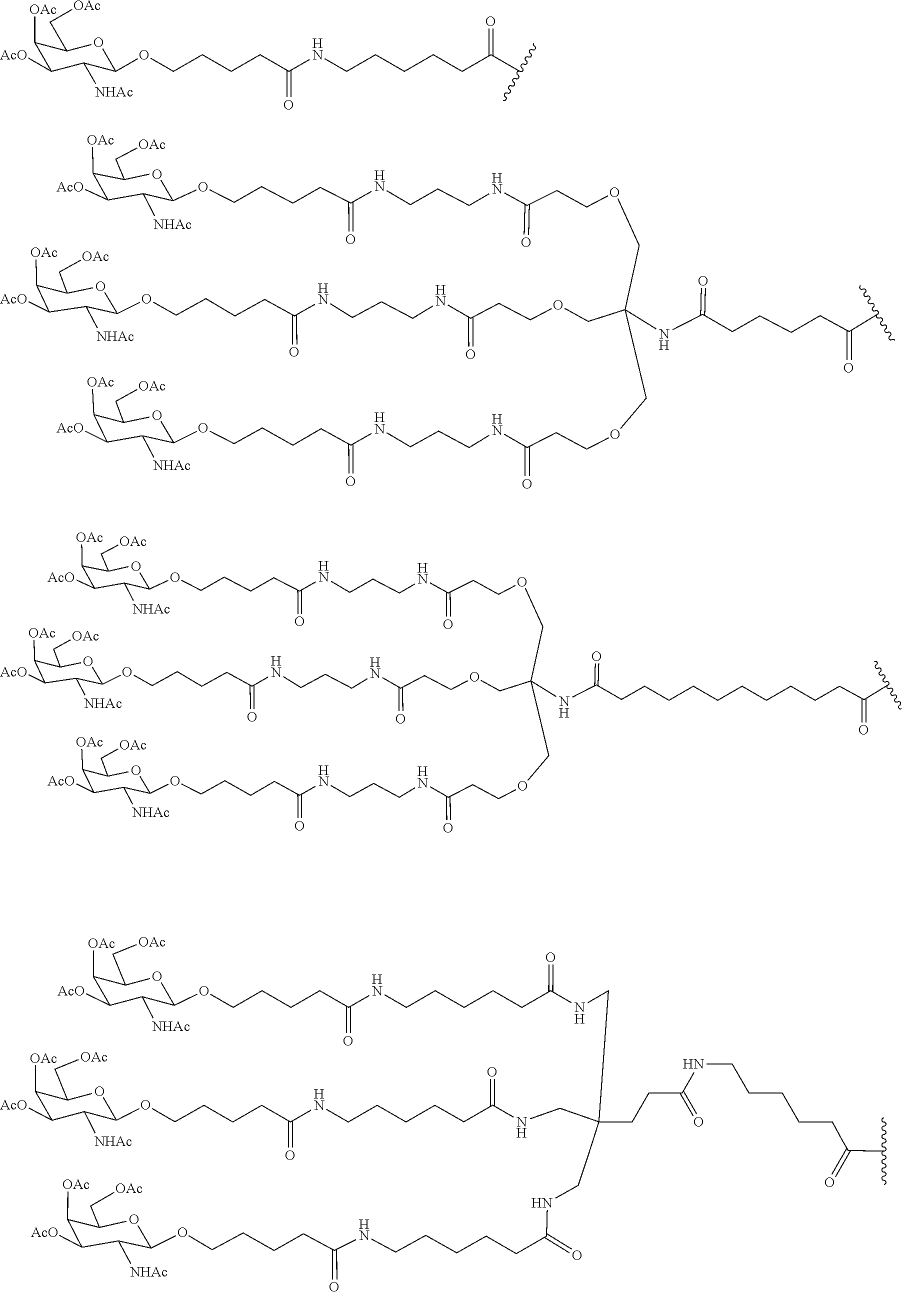

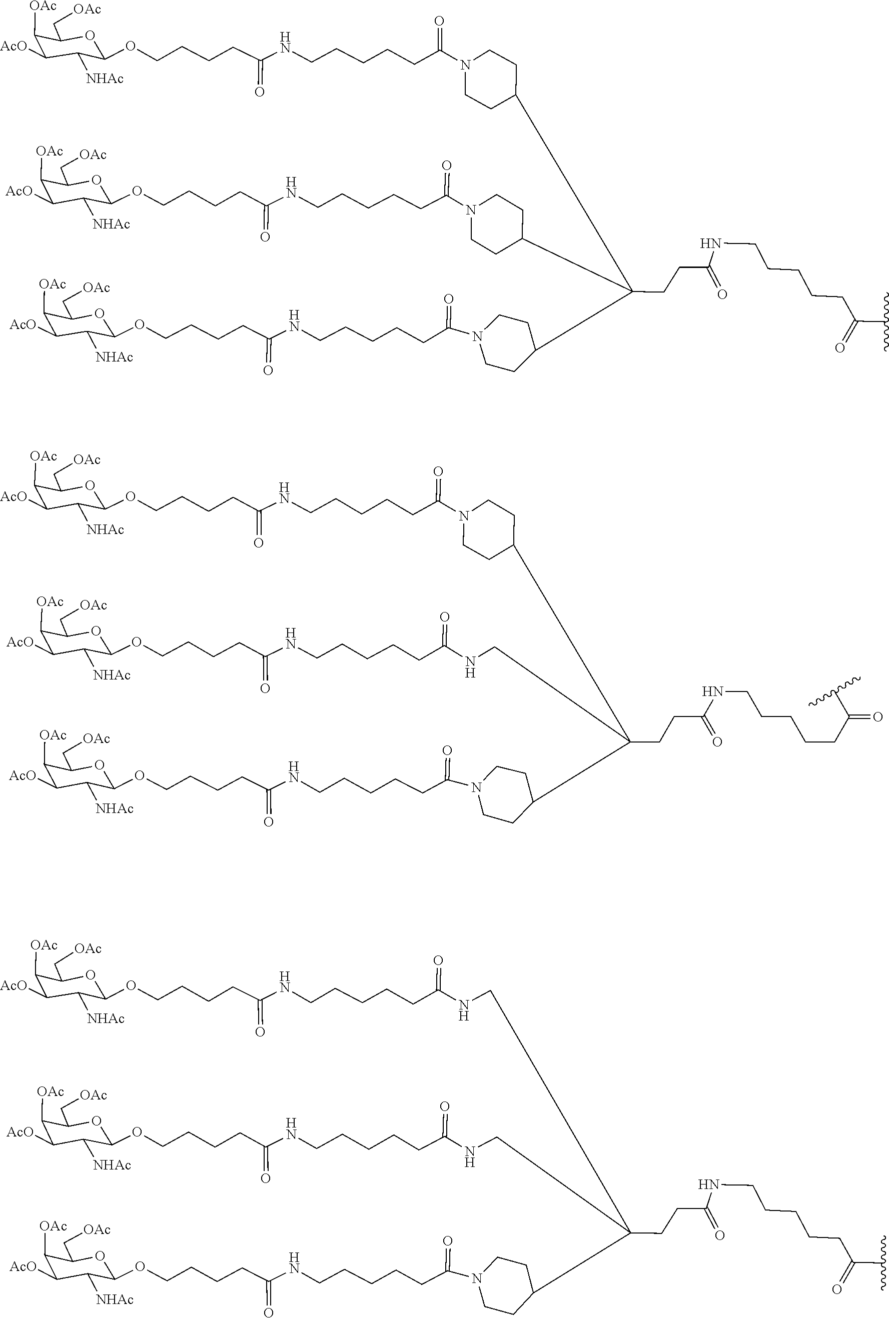

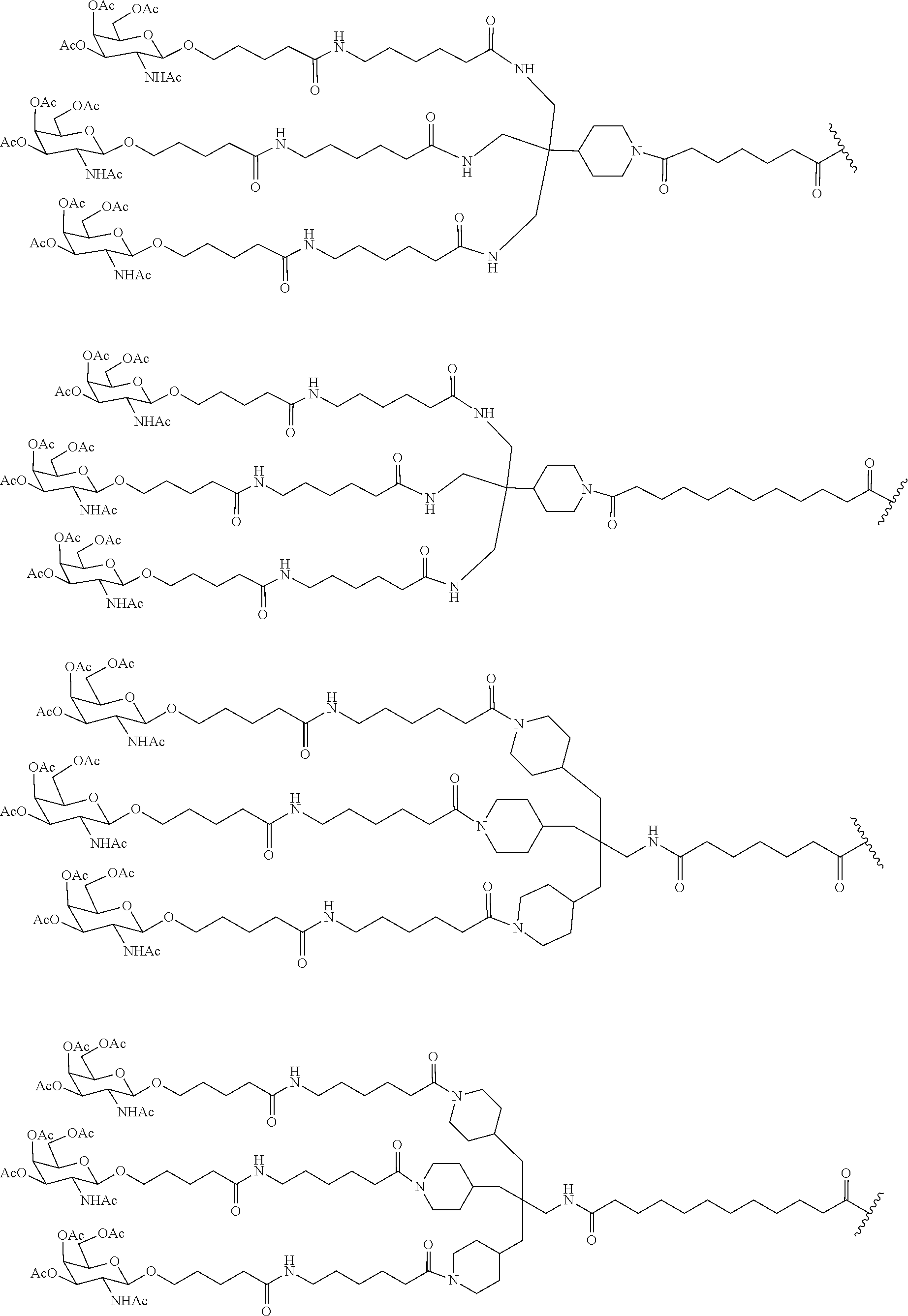

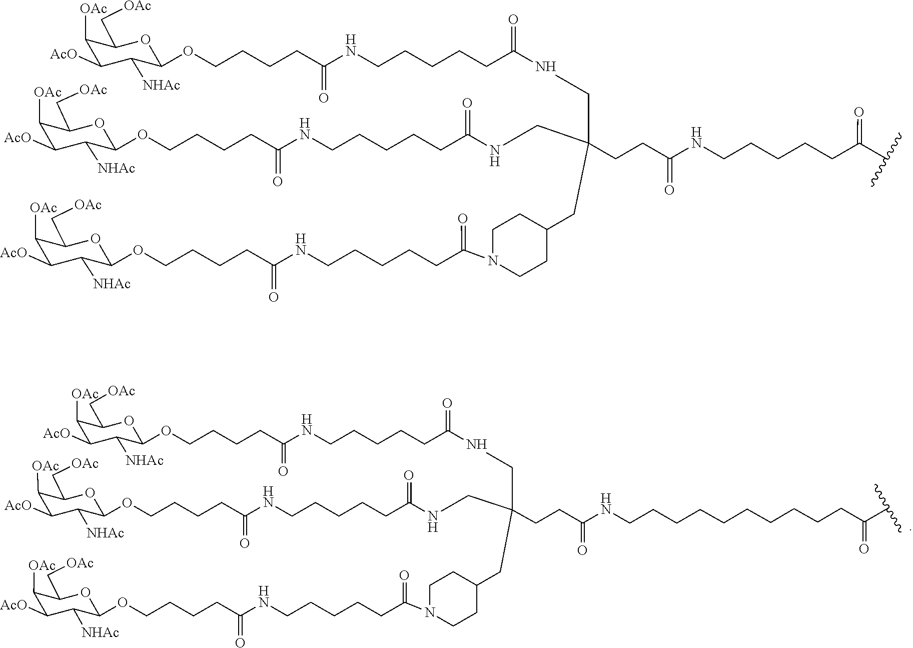 [SMILES: *C(=O)CCCCCNC(=O)CCC(CNC(=O)CCCCCNC(=O)CCCCOC1OC(COC(C)=O)C(OC(C)=O)C(OC(C)=O)C1NC(C)=O)(CNC(=O)CCCCCNC(=O)CCCCOC1OC(COC(C)=O)C(OC(C)=O)C(OC(C)=O)C1NC(C)=O)CC1CCN(C(=O)CCCCCNC(=O)CCCCOC2OC(COC(C)=O)C(OC(C)=O)C(OC(C)=O)C2NC(C)=O)CC1 ; *C(=O)CCCCCCCCCCNC(=O)CCC(CNC(=O)CCCCCNC(=O)CCCCOC1OC(COC(C)=O)C(OC(C)=O)C(OC(C)=O)C1NC(C)=O)(CNC(=O)CCCCCNC(=O)CCCCOC1OC(COC(C)=O)C(OC(C)=O)C(OC(C)=O)C1NC(C)=O)CC1CCN(C(=O)CCCCCNC(=O)CCCCOC2OC(COC(C)=O)C(OC(C)=O)C(OC(C)=O)C2NC(C)=O)CC1]

In some embodiments, the targeting ligand is:

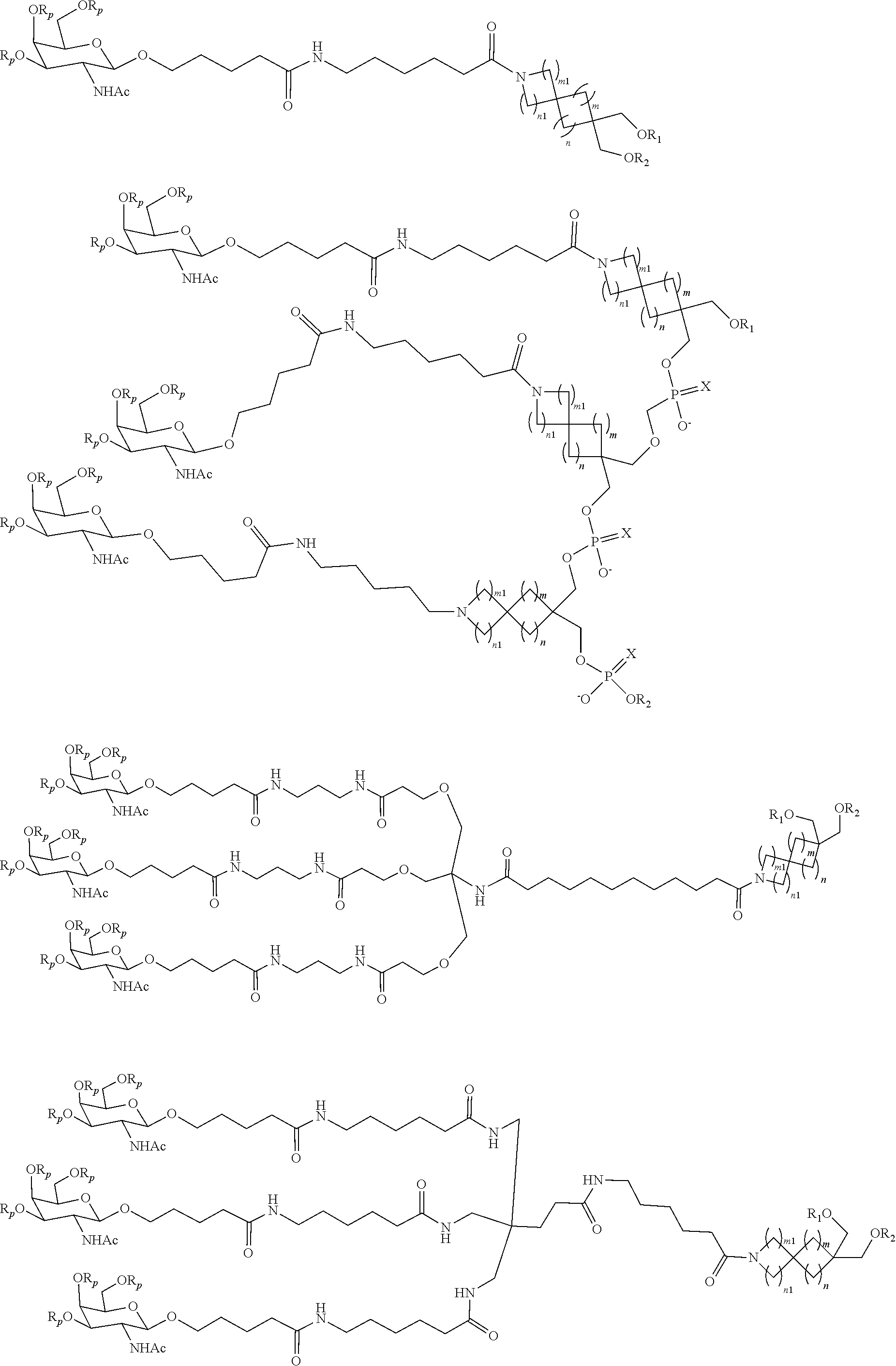

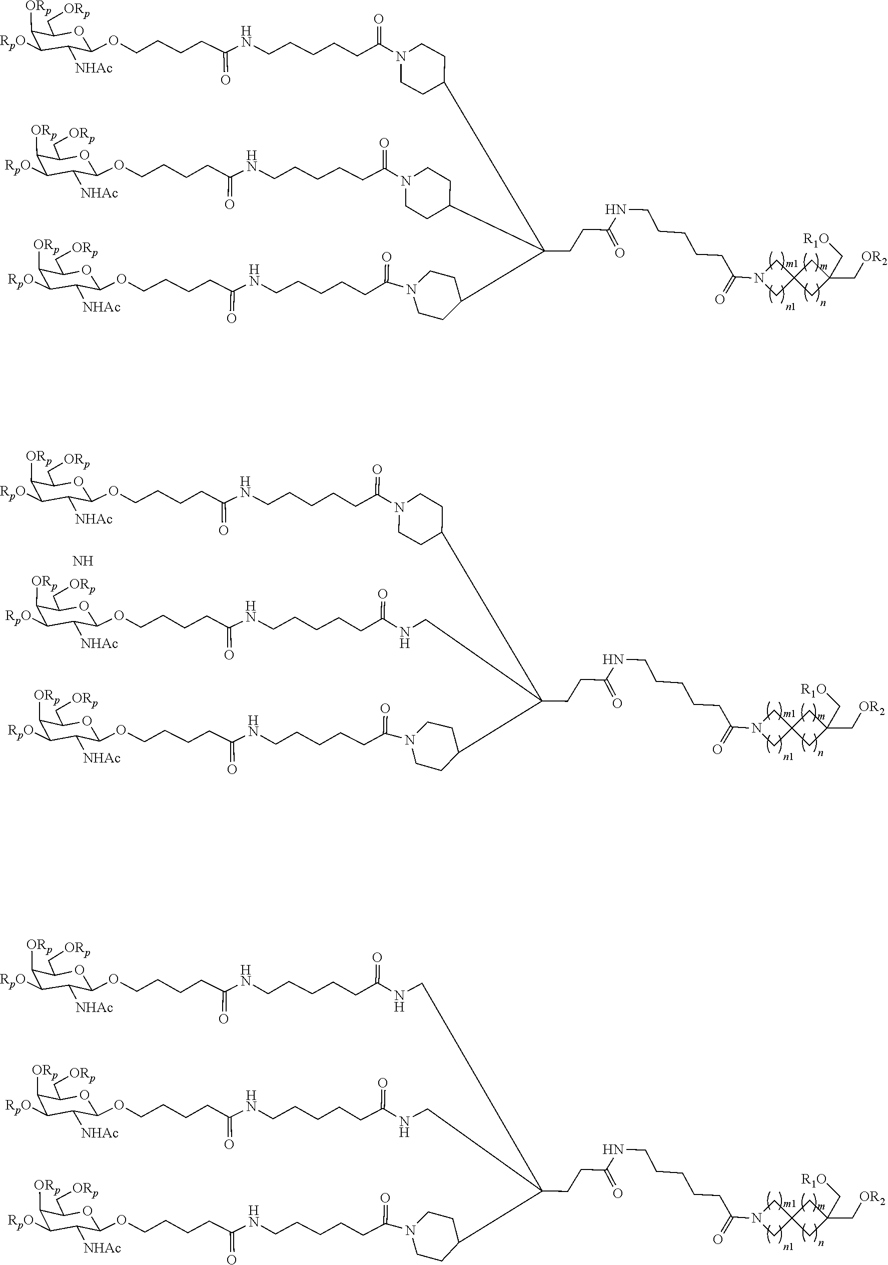

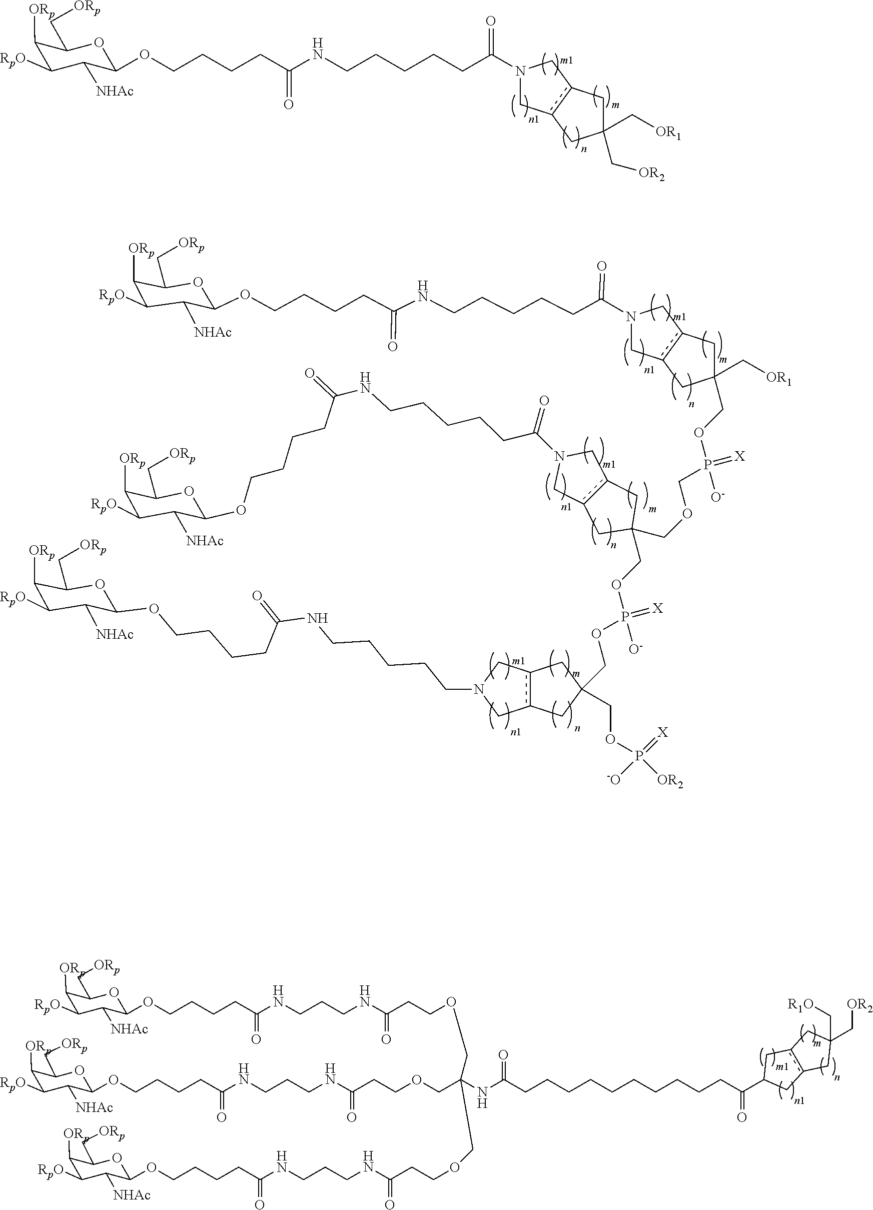

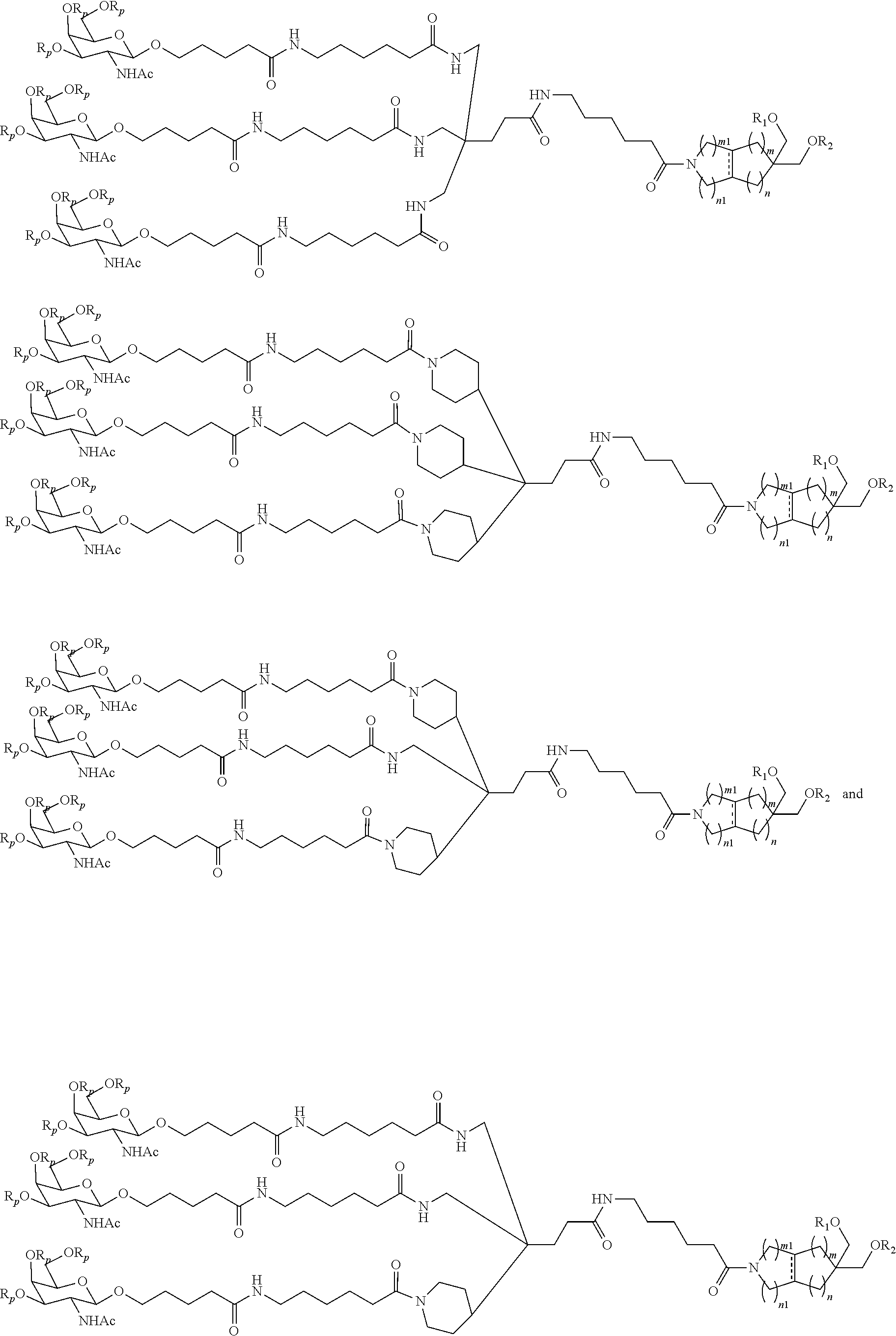

Rₚ is H or hydroxyl protecting group, preferably, hydroxyl protecting group is acetyl;
X is O or S;
each of m and n is independently selected from 1 or 2;
each of m1 and n1 is independently selected from 1, 2 or 3.

In some embodiments, the R₁ is hydrogen.

In some embodiments, R¹ is substituted or unsubstituted triphenylmethyl hydroxyl protecting group.

In some embodiments, R¹ is

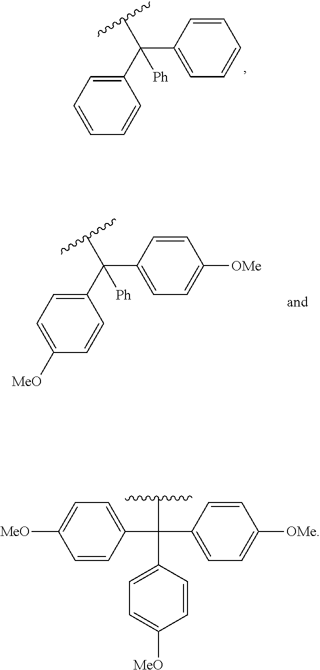 [SMILES: *C(c1ccccc1)(c1ccccc1)c1ccccc1 ; *C(c1ccccc1)(c1ccc(OC)cc1)c1ccc(OC)cc1 ; *C(c1ccc(OC)cc1)(c1ccc(OC)cc1)c1ccc(OC)cc1]

In some embodiments, R¹ is

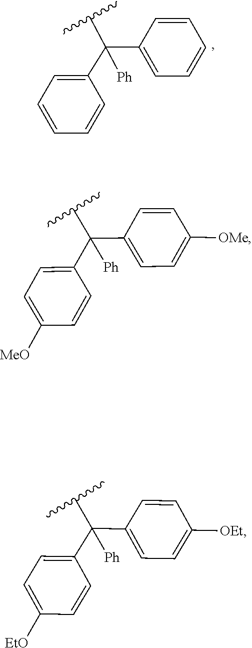 [SMILES: *C(c1ccccc1)(c1ccccc1)c1ccccc1 ; *C(c1ccccc1)(c1ccc(OC)cc1)c1ccc(OC)cc1 ; *C(c1ccccc1)(c1ccc(OCC)cc1)c1ccc(OCC)cc1]

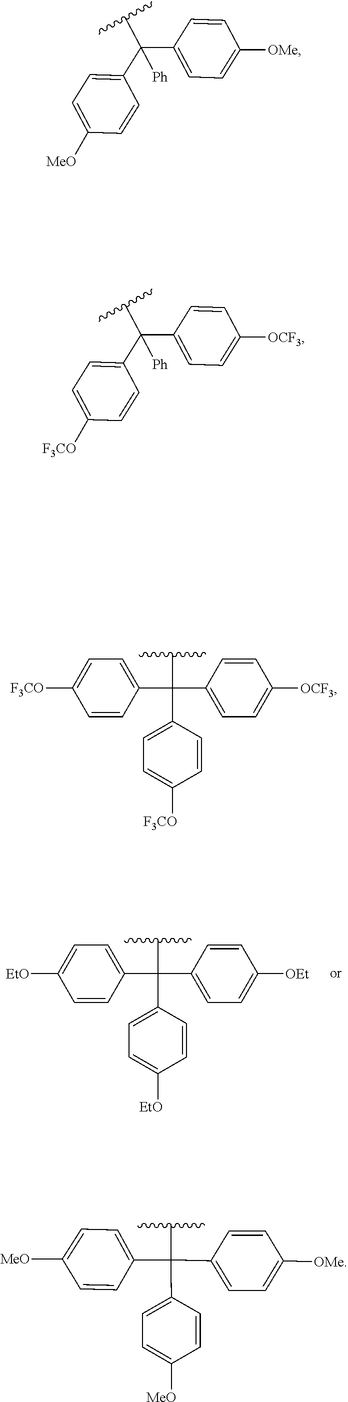 [SMILES: *C(c1ccccc1)(c1ccc(OC)cc1)c1ccc(OC)cc1 ; *C(c1ccccc1)(c1ccc(OC(F)(F)F)cc1)c1ccc(OC(F)(F)F)cc1 ; *C(c1ccc(OC(F)(F)F)cc1)(c1ccc(OC(F)(F)F)cc1)c1ccc(OC(F)(F)F)cc1 ; *C(c1ccc(OCC)cc1)(c1ccc(OCC)cc1)c1ccc(OCC)cc1 ; *C(c1ccc(OC)cc1)(c1ccc(OC)cc1)c1ccc(OC)cc1]

In some embodiments, R₂ is hydrogen.

In some embodiments, R₂ is

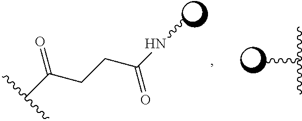

is a solid support.

In some embodiments, R₂ is

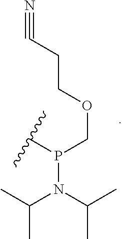 [SMILES: *P(COCCC#N)N(C(C)C)C(C)C]

The targeting ligand of the present invention can be linked to a therapeutic agent, for use as medicament. The therapeutic agent is selected from small molecule drug and macromolecular drug.

In a preferred embodiment, the therapeutic agent is siRNA.

The present invention, in another aspect, also provides spiro and fused bicyclic heterocycles of formula (V) and (VI). Bicyclic compounds are molecules that contain two rings that are joined together. There are three different ways that rings can be joined, including (1) fused bicyclics-two atoms and the bond between them are shared; (2) bridged bicyclics-more than two atoms are shared; (3) spirocyclics-two rings are joined at a single atom. Bicyclic compounds could be fully carbocyclic (all carbon) or heterocyclic (having one or more non-carbon atoms). Bicyclic heterocycles are key intermediates in drug synthesis which can be classified as nitrogen-, sulfur- and oxygen-containing compounds. Bicyclic heterocycles are of recent interest because of their interesting conformational features and their structural implications for biological systems. The strained bicyclic compounds are important targets, as they show diverse new biological activities for the unique shapes and properties. Moreover, besides potentially modulating binding potency and specificity, bicyclic scaffolds may also improve bioavailability and metabolic stability, key issues when developing new pharmacological entities. This patent has designed a series of novel spiro and fused bicyclic heterocycles with multiple functional groups as shown below. These spiro and fused bicyclic compounds could be used to tether different parts such as GalNAc, oligonucleotide and solid support.

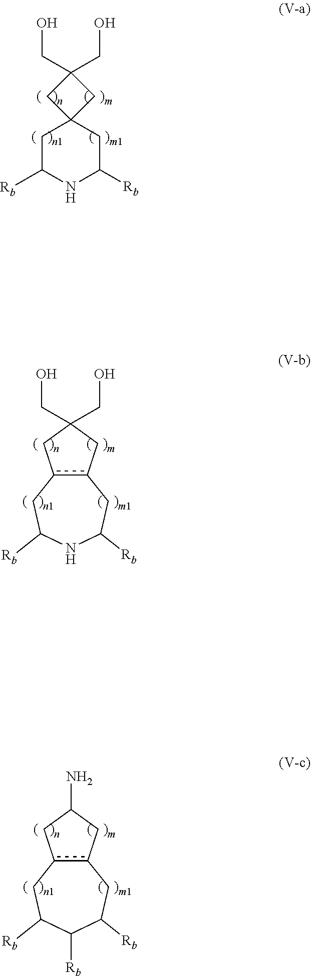

[symbol] is a single or double bond;
R_{b} is independently selected from the group of hydrogen and oxo;
each of m and n is independently selected from 1, 2, 3; 4 and 5
each of m1 and n1 is each independently selected from 0, 1, 2, 3 and 4.

The present invention, in another aspect, also provides bicyclic heterocycles of formula (VI-a), (VI-b), or (VI-c), or tautomer, pharmaceutically acceptable salt:

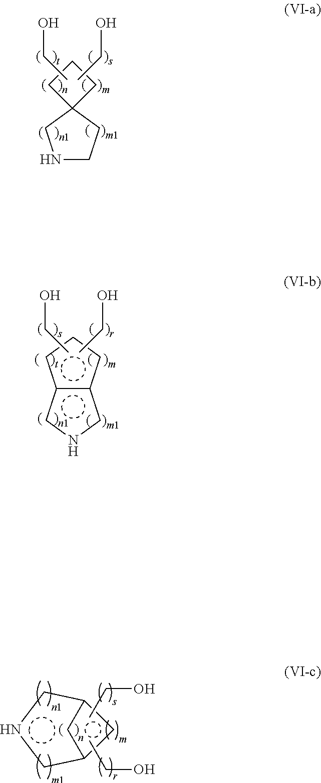

m is independently selected from 0, 1, 2, 3; 4 and 5;
n is independently selected from 1, 2, 3; 4 and 5;
each of r, s and t is independently selected from 0, 1 and 2;
each of m1 and n1 is each independently selected from 0, 1, 2, 3 and 4.

In a preferred embodiment, the compound selected from the following structure:

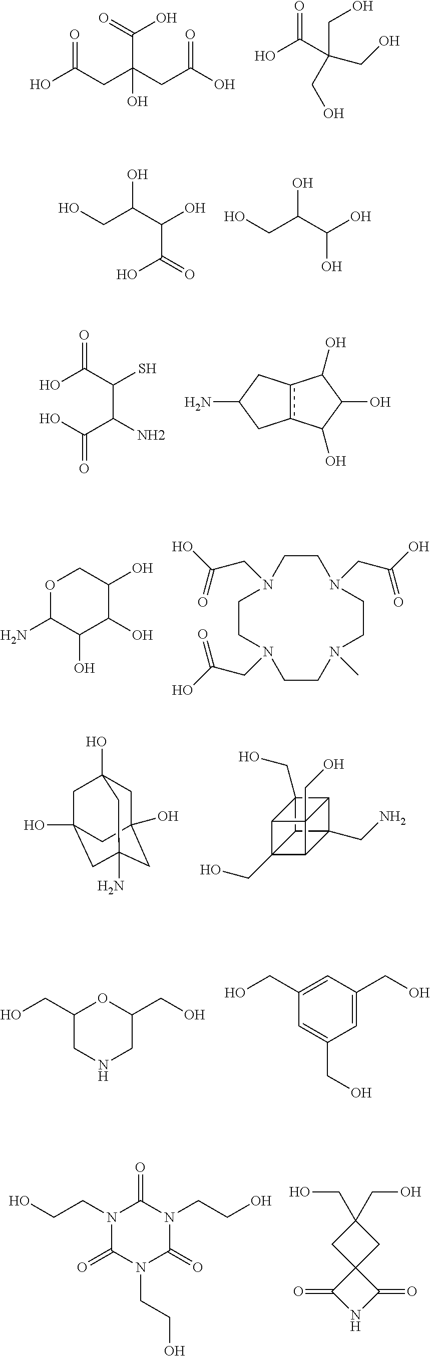

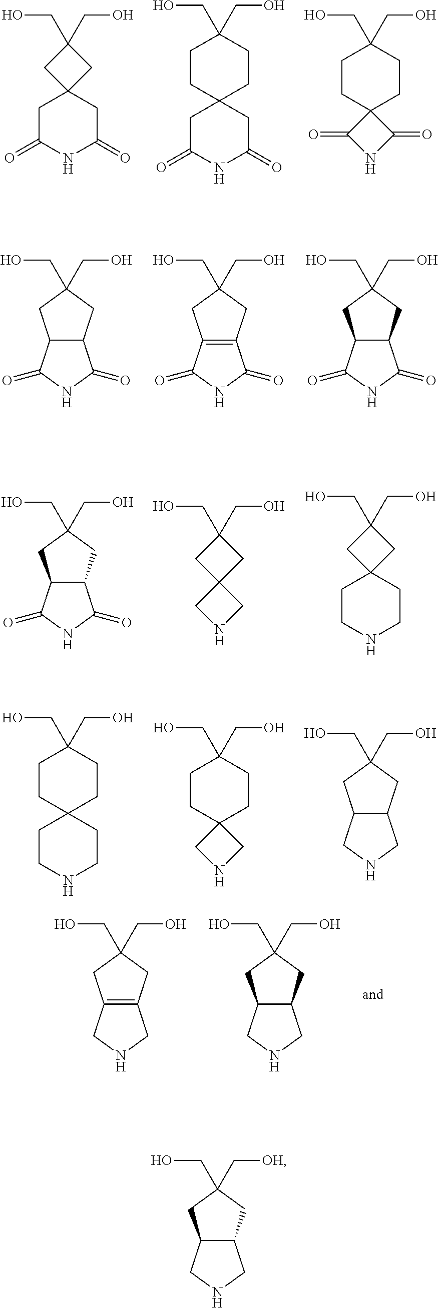

or tautomer, pharmaceutically acceptable salt thereof.

### GalNAc-Spiro/Fused Bicyclic Conjugation

GalNAc could be conjugated to different modalities by different linkers and tethering groups. The Spiro/bicyclic compounds could provide unique conformation to the whole conjugation. To introduce GalNAc to oligonucleotides, GalNAc ligands could be first assembled as a single piece of so-called triantennary ligand, then conjugated to solid support or transferred to its phosphoramidite form. However, an alternative approach using a monovalent GalNAc support with two additions of a monovalent GalNAc phosphoramidite was also described and yielded a structure shown below. This (1+1+1) trivalent GalNAc structure led to GalNAc modified siRNA oligos with potency equal to the equivalent siRNA with the triantennary GalNAc ligand both in vitro and in vivo.

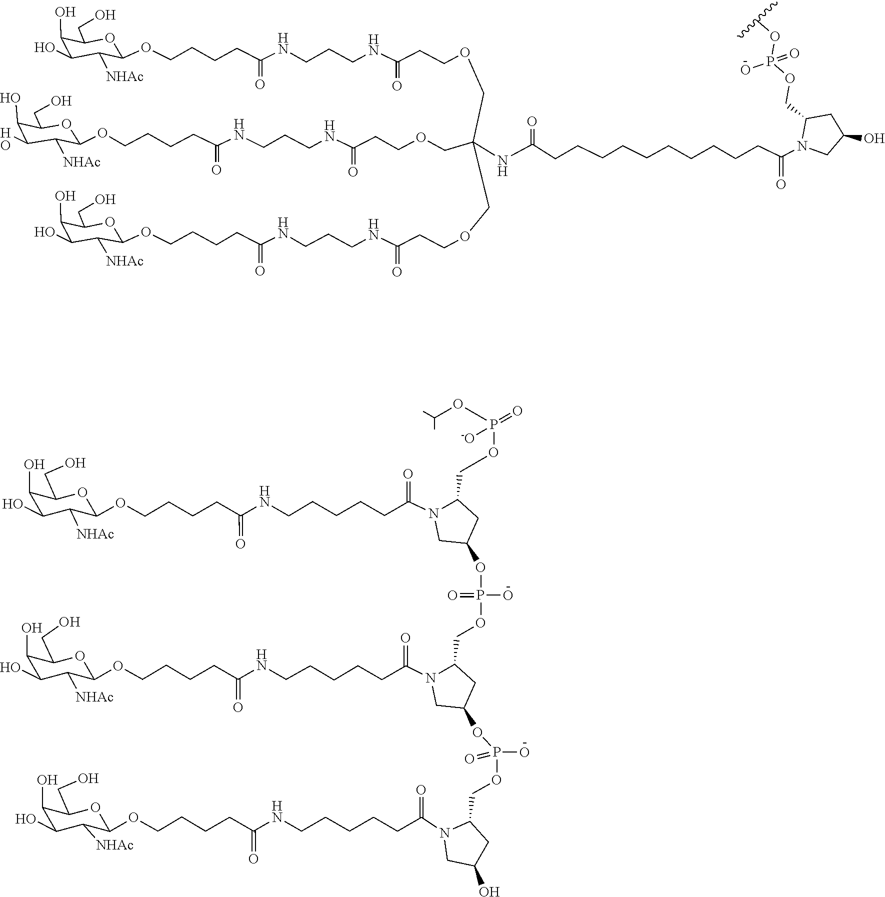

The present invention provides a series of novel GalNAc-bicyclic compounds, which could be conjugated to oligonucleotides by amidite chemistry or loaded onto solid support for solid phase oligonucleotide synthesis, for example:

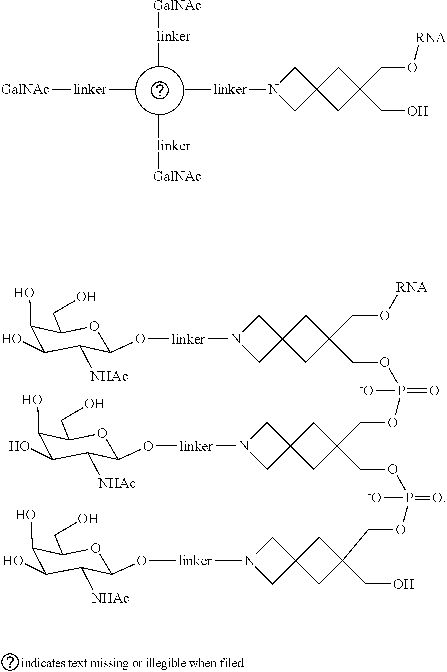

The tetra functional group is selected from

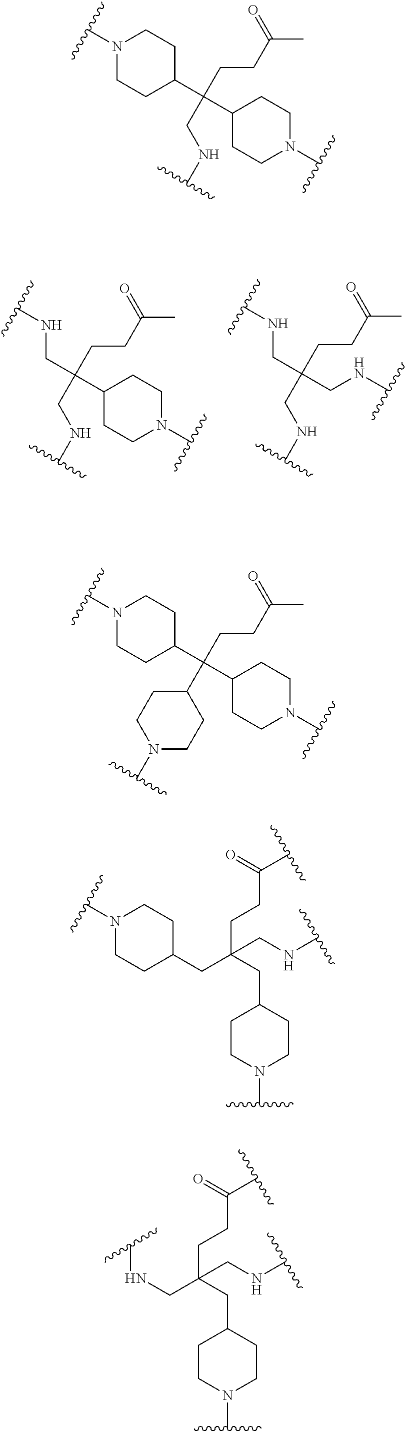 [SMILES: *NCC(CCC(C)=O)(C1CCN(*)CC1)C1CCN(*)CC1 ; *NCC(CCC(C)=O)(CN*)C1CCN(*)CC1 ; *NCC(CCC(C)=O)(CN*)CN* ; *N1CCC(C(CCC(C)=O)(C2CCN(*)CC2)C2CCN(*)CC2)CC1 ; *NCC(CCC(*)=O)(CC1CCN(*)CC1)CC1CCN(*)CC1 ; *NCC(CCC(*)=O)(CN*)CC1CCN(*)CC1]

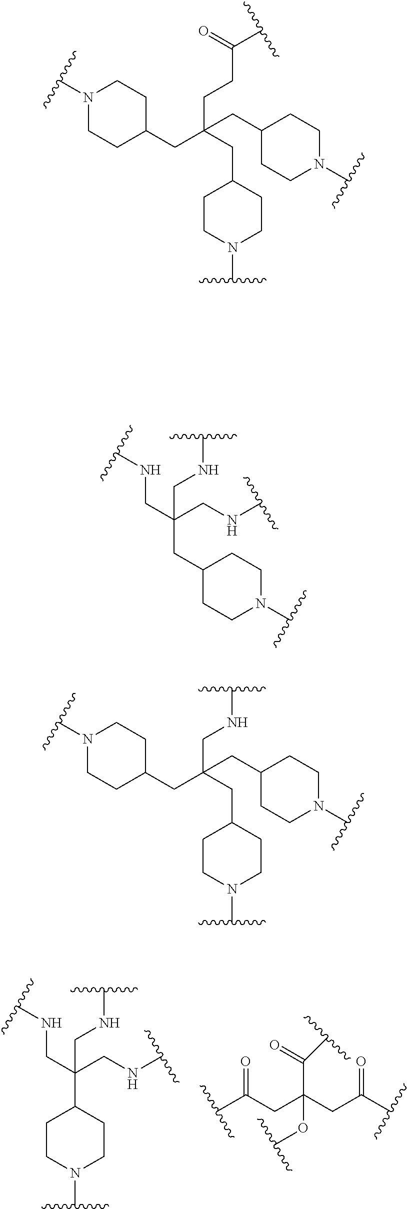

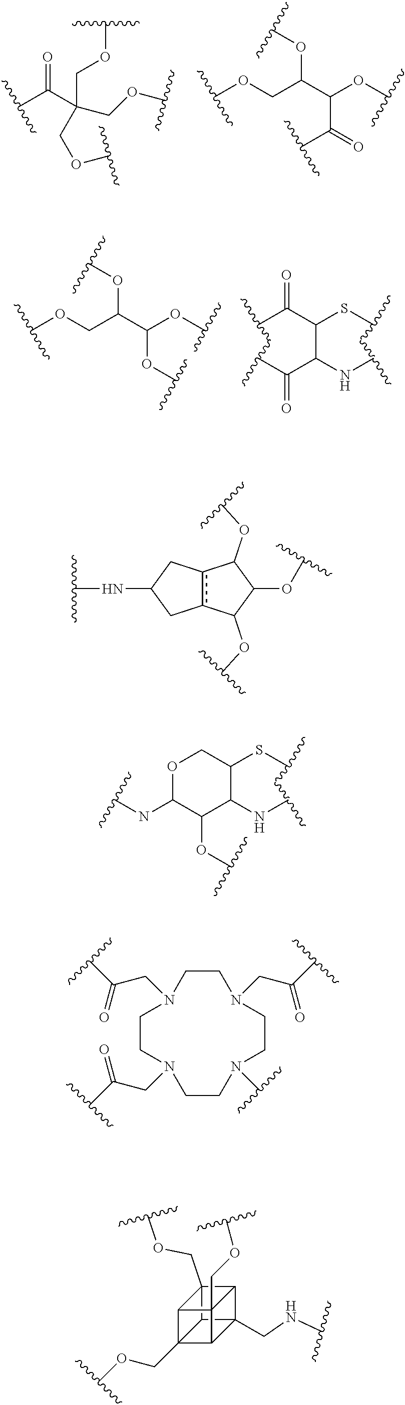

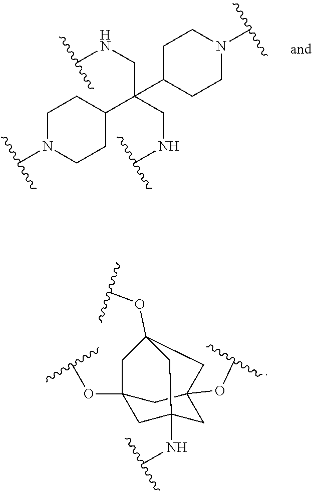 [SMILES: *NCC(CN*)(C1CCN(*)CC1)C1CCN(*)CC1 ; *NC12CC3(O*)CC(O*)(C1)CC(O*)(C2)C3]

In another aspect, the present invention provides a therapeutic compound comprising at least one therapeutic oligonucleotide, and the oligonucleotide conjugate to the targeting ligand.

In some embodiments, the targeting ligand conjugate to the 3′ end or the 5′ end of oligonucleotide; more preferably, the oligonucleotide is siRNA; more preferably, the siRNA is C3, C5, CFB, PCSK9, TTR, AGT, LPA, Agtr1, ALK, VEGF, ANGPTL3, ANGPTL4, ANGPTL8, APOA, APOC3, ASGR1, CIDEB, COL1A1, COL3A1, CTGF, DGAT2, DMPK, DNAJC15/MCJ, DPP4, Factor VIII, Factor X, Factor IX, Factor XI, Factor XII, GPR146, GPR75, GRB10/14, TLR7/8/RIG-1, HSD17B13, INHBE, ITGV6, KHK, KLK1, MASP2, MTARC1, MUC5B, NPC1L1, PNPLA3, ASGR1, SCAP, SERPINA1, SERPINF2, SREBF2, HMGCR, TGFB1, COX-2, TP53, CD4, CD8, CD40, CD71, DUX4, XDH, LDHA, ALDH2, DMD, EPHA2, KIF11, BCL2L12, APOA1, TRPV1, CASP2, KRAS, TMPRSS6, STAT3, PRDM14, PTGS2, CTGF, DDIT4, siRNA.

The novel GalNAc conjugates of the present invention have better activity compared to the prior GalNAc conjugates in vitro and in vivo.

The present invention also provides a pharmaceutical composition comprising the therapeutic compound or pharmaceutically acceptable salt thereof, and a pharmaceutically acceptable carrier, diluent or excipients.

In another aspect, the present invention relates to a method of treating a disease or disorder, comprising administering to a subject in need thereof an effective amount of the therapeutic compound or a pharmaceutically acceptable salt thereof, or a pharmaceutical composition comprising the same.

In some embodiments, the disease or disorder is liver disease, genetic disease, bleeding disorder (e.g. hemophilia), rare disease (e.g. acromegaly), metabolic diseases (e.g. hypercholesterolemia, dyslipidemia, hypertriglyceridemia), cardiovascular diseases, obesity, hemochromatosis, alcohol dependence and anemia (e.g. anemia of chronic disease, thalassemia).

In some embodiments, the liver disease is liver injury, liver fibrosis, alcoholic liver diseases, non-alcoholic steotohepatitis (NASH), non-alcoholic fatty liver disease (NAFLD) or viral hepatitis.

In another aspect, the present invention relates to a method of synthesizing the therapeutic compound, comprising: providing the targeting ligand of formula (I), and conjugating the targeting ligand to an oligonucleotide.

In a preferred embodiment, the targeting ligand be conjugated to the 3′ end or the 5′ end of oligonucleotide.

In a preferred embodiment, the targeting ligands could be conjugated to the 3′ end and the 5′ end of oligonucleotide

As described previously, GalNAc could be conjugated to oligonucleotide as a single could piece or step by step (1+1+1).

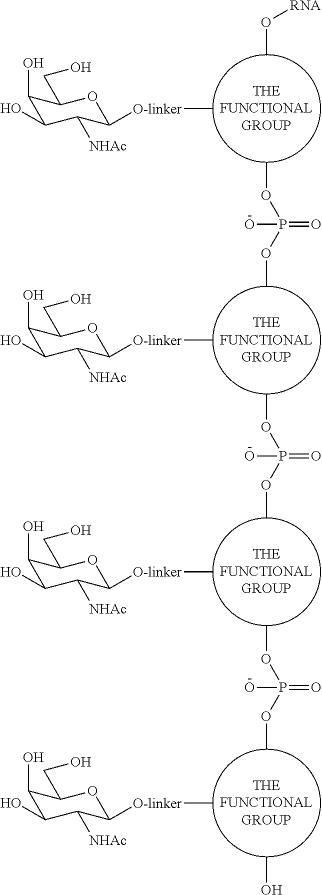

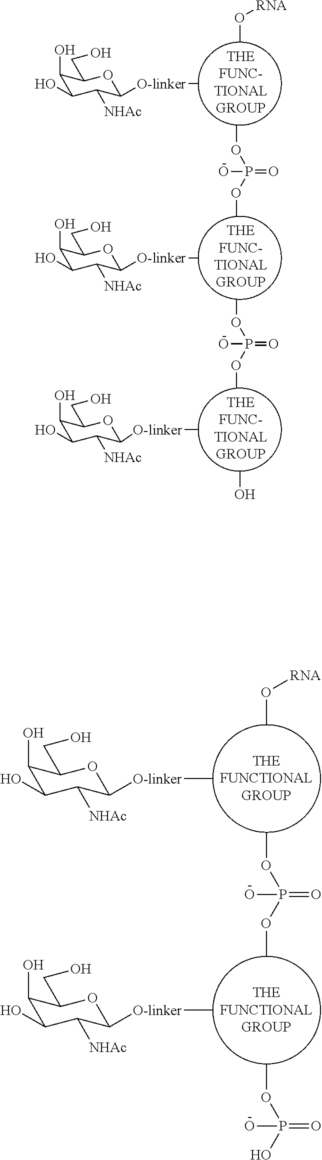

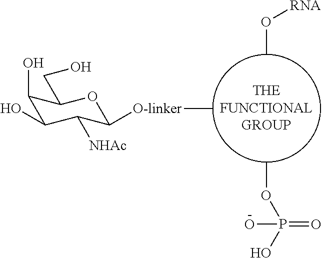

To realize the 1+1+1 assembly, mono-GalNAc amidite and mono-GalNAc solid support should be synthesized.

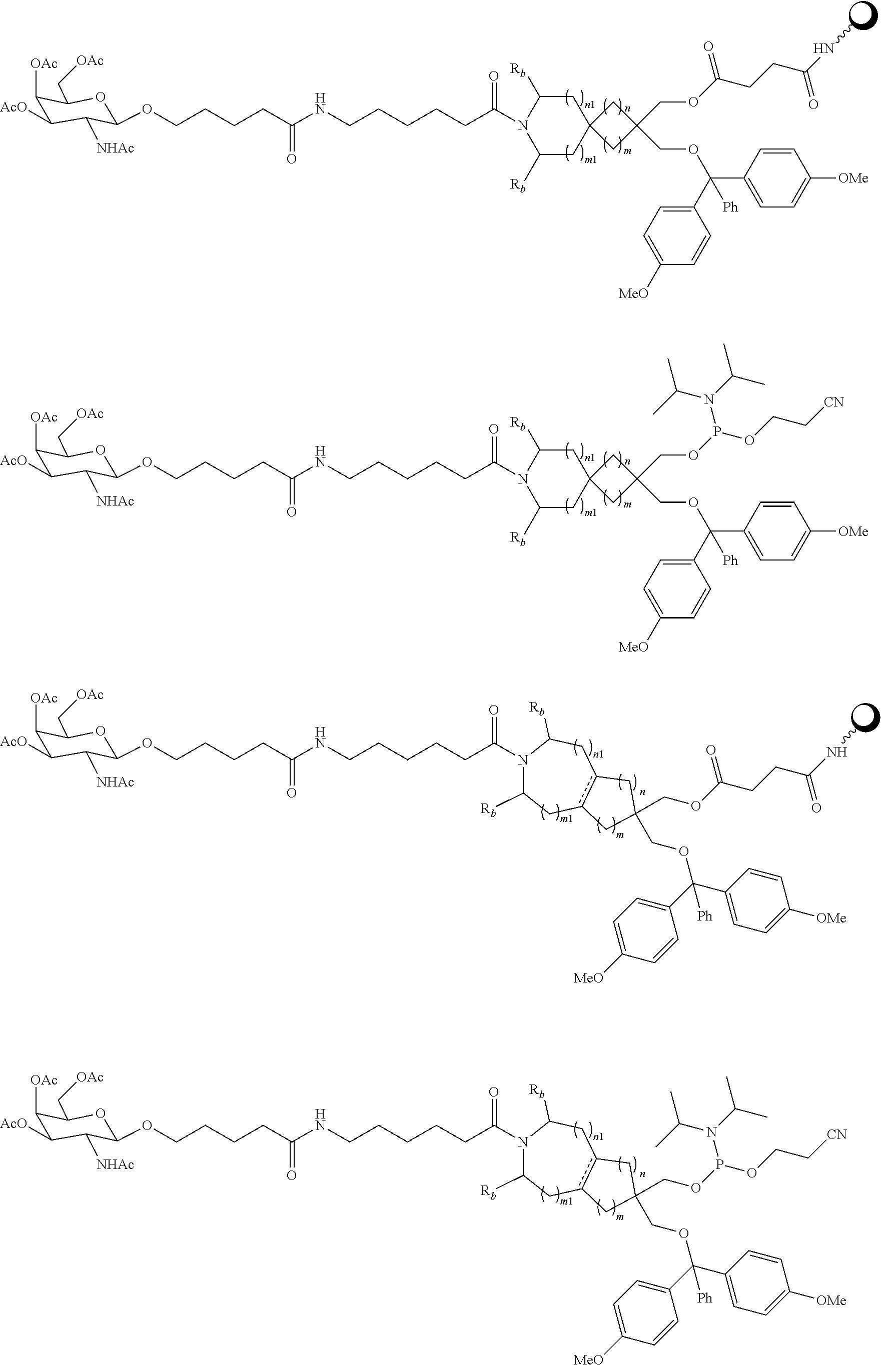

R_{b}, m, n, m1 and n1 are defined as in formula (V-a) and (V-b).

The specific method is as follows,

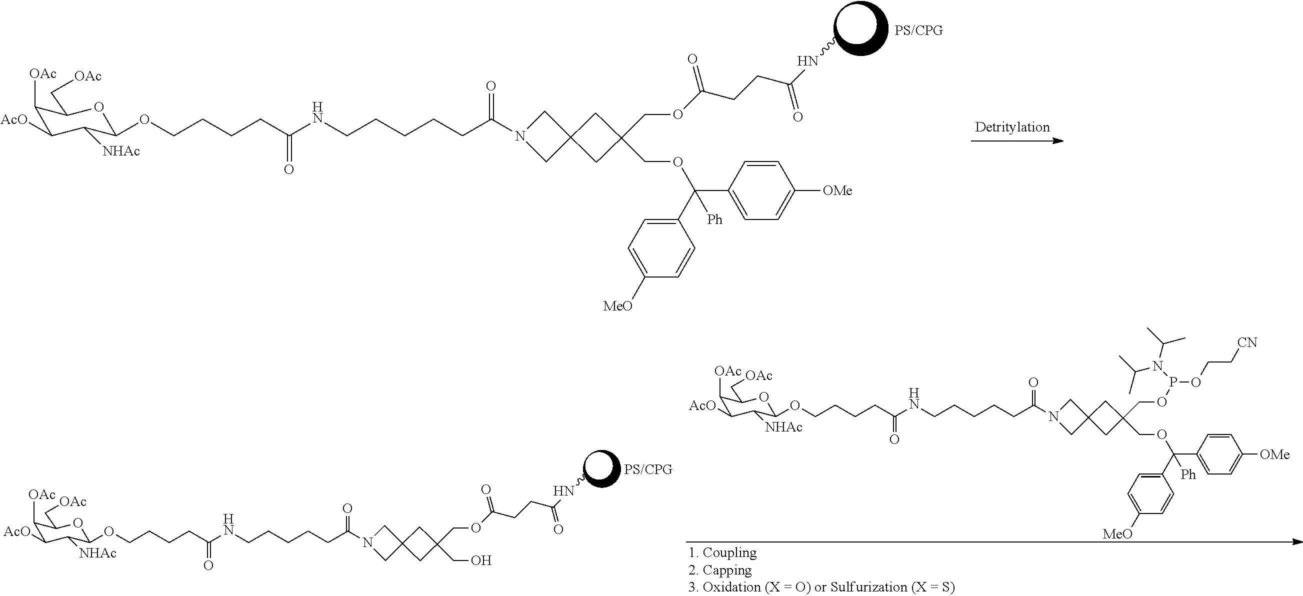

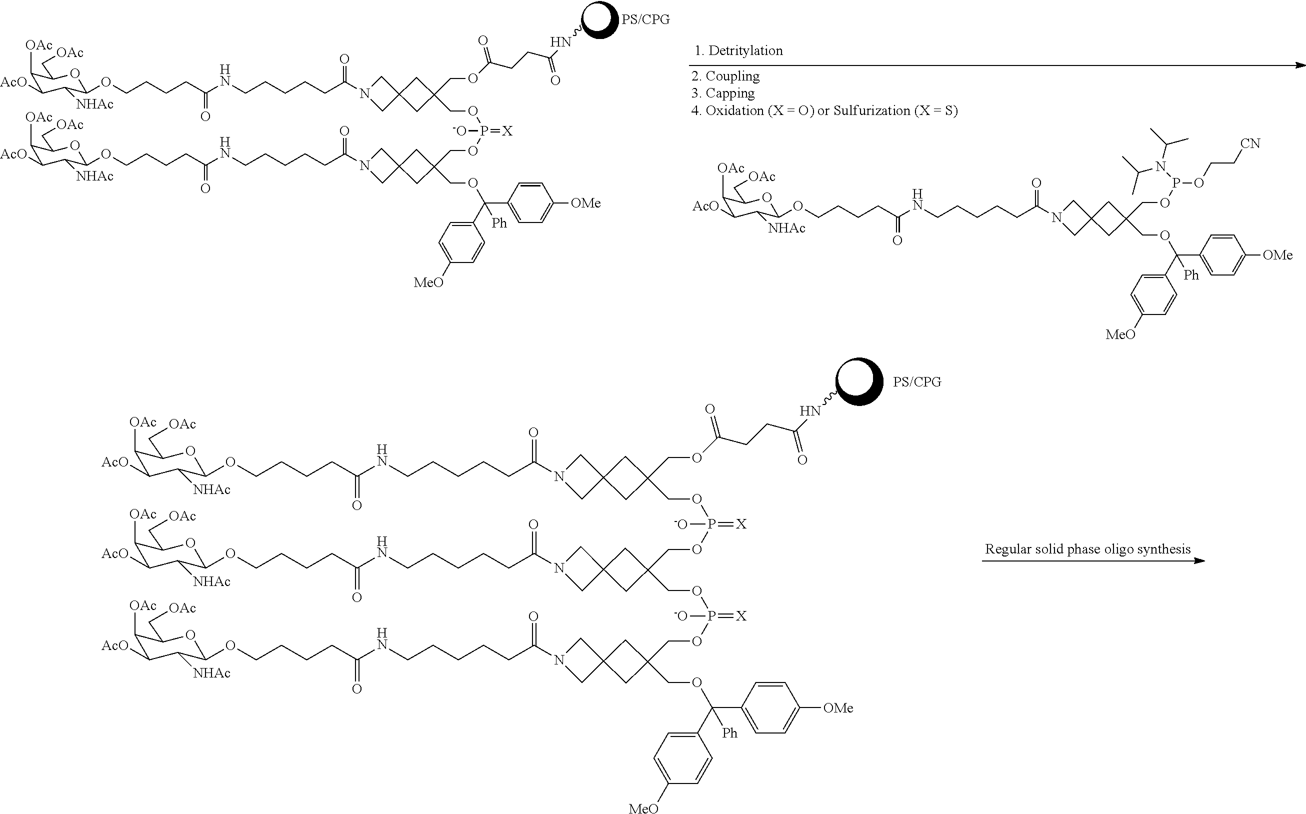

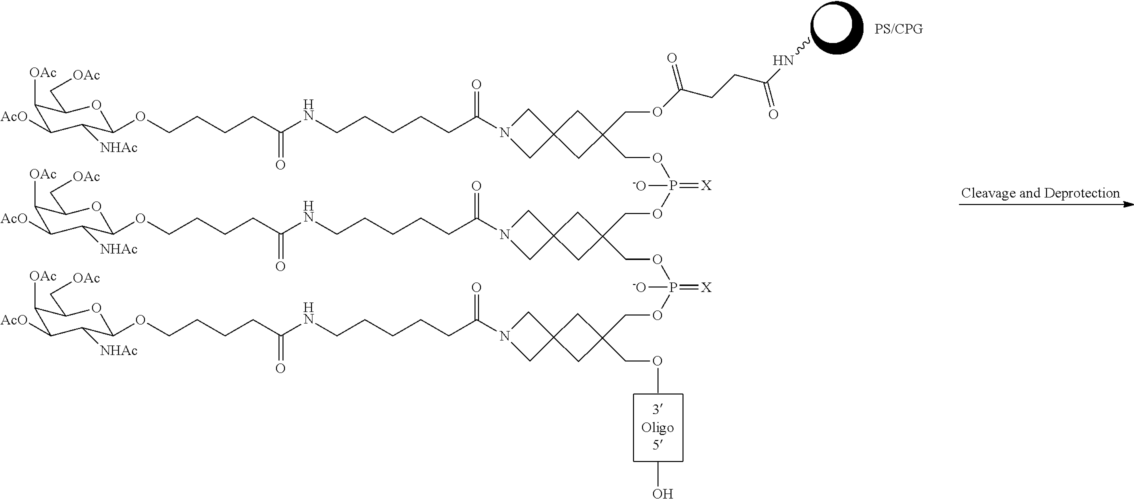

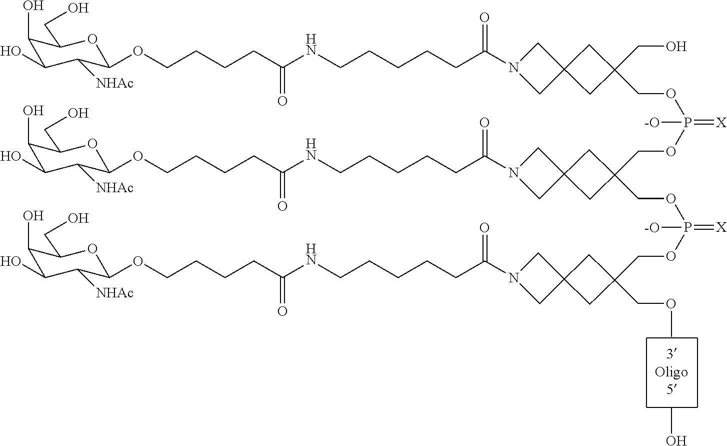

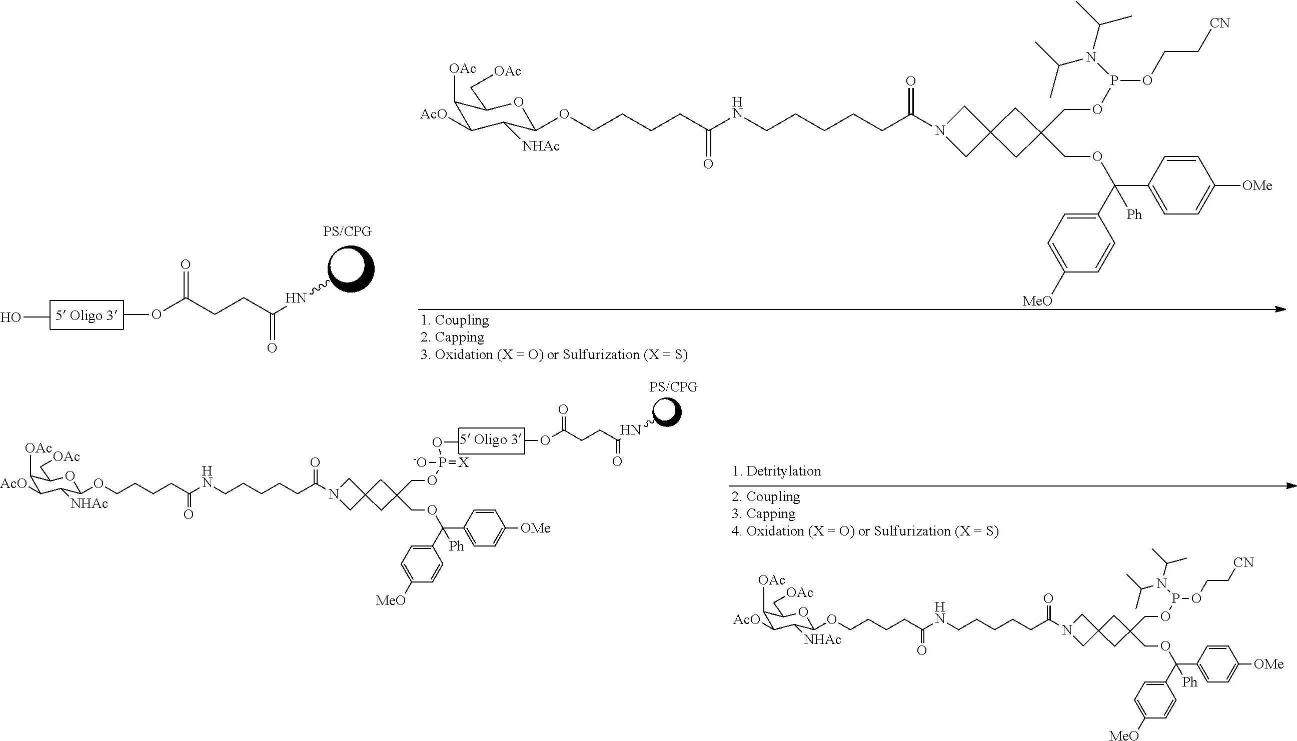

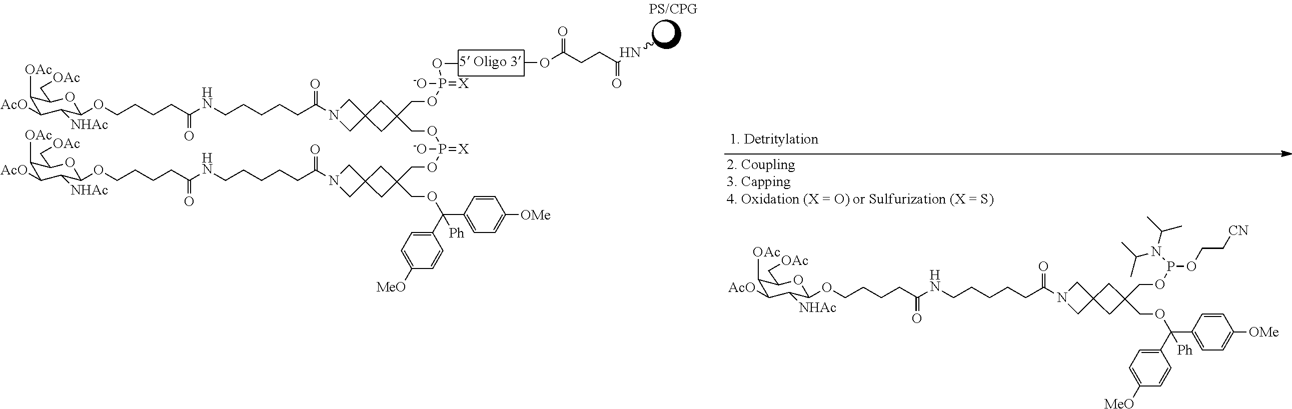

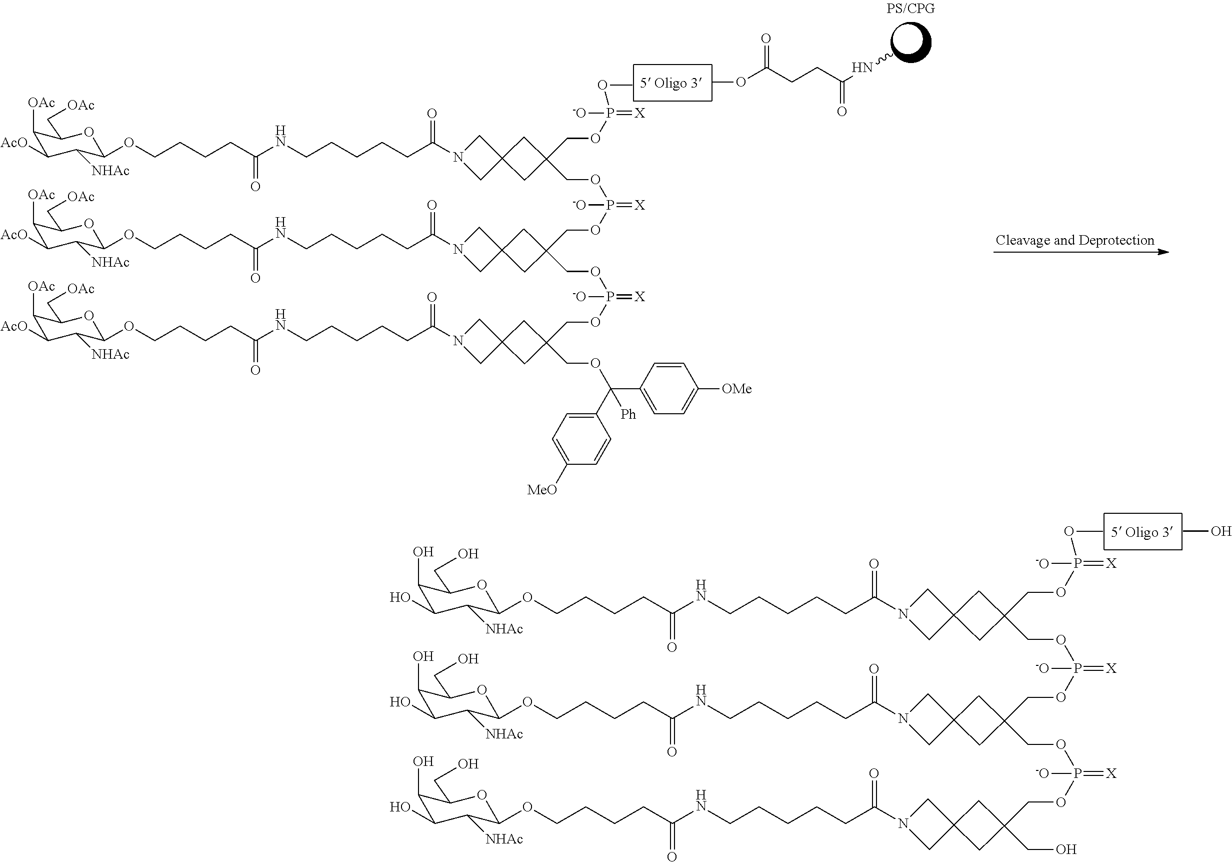

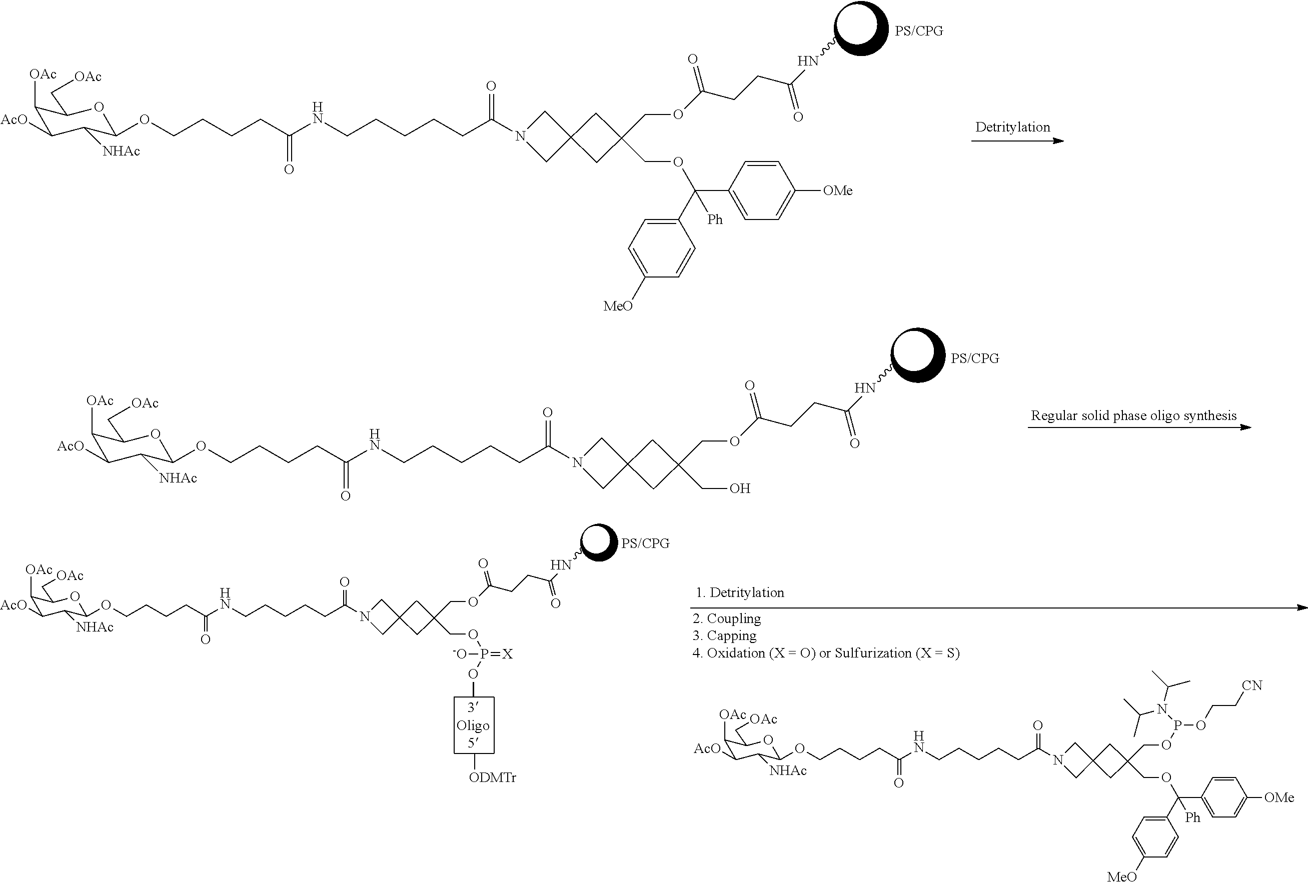

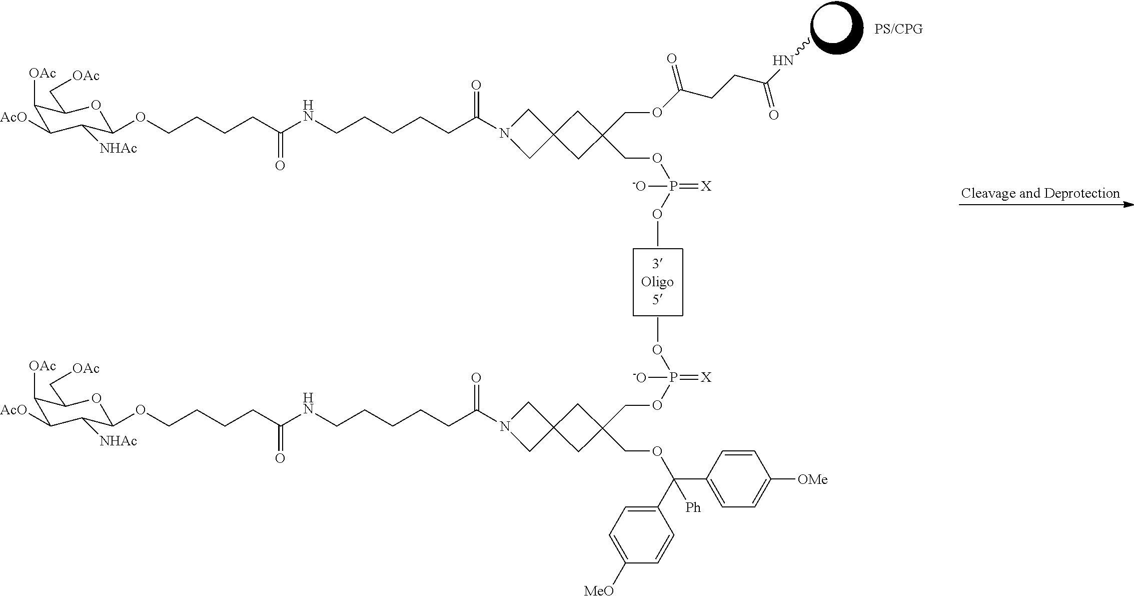

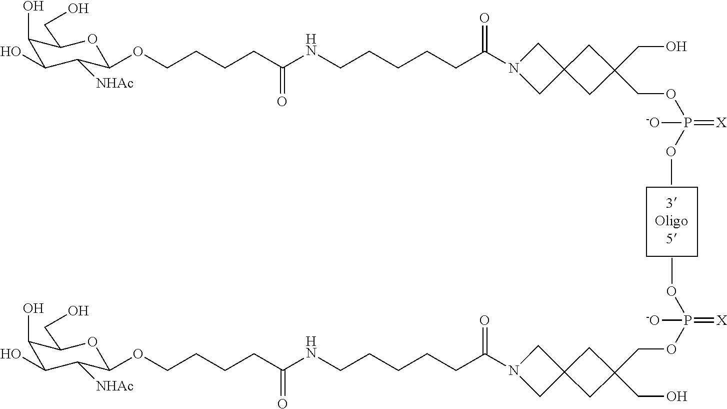

### BRIEF DESCRIPTION OF THE DRAWINGS

FIG. 1. illustrating the effect of siRNA-24, and siRNA-25 on serum mC3 protein in vivo studies.

FIG. 2. illustrating the effect of siRNA-24, and siRNA-25 on liver mC3 mRNA levels in vivo studies.

### DETAILED DESCRIPTION OF THE INVENTION

Given below are definitions of terms used in this application. Any term not defined herein takes the normal meaning as the skilled person would understand the term. ‘Alkyl’ refers to a saturated aliphatic hydrocarbon group including C₁-C₂₀ straight chain and branched chain groups. Preferably an alkyl group is an alkyl having 1 to 12, sometimes preferably 1 to 6, sometimes more preferably 1 to 4, carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

‘Alkoxy’ refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

‘Alkylthio’ refers to both an —S-(alkyl) and an —S— (unsubstituted cycloalkyl) group, wherein the alkyl is defined as above.

‘Haloalkyl’ refers to an alkyl group substituted by a halogen group, wherein alkyl is as defined above.

‘Halogen’ refers to fluoro, chloro, bromo or iodo atoms.

‘Cycloalkyl’ refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 carbon atoms or 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

‘Spiro Cycloalkyl’ refers to a 5 to 20 membered polycyclic group with rings connected through one common carbon atom (called a spiro atom), wherein one or more rings can contain one or more double bonds, but none of the rings has a completely conjugated pi-electron system. Preferably a spiro cycloalkyl is 6 to 14 membered, and more preferably 7 to 10 membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following substituents:

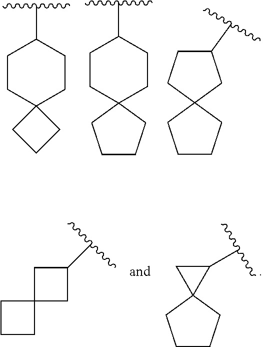

‘Fused Cycloalkyl’ refers to a 5 to 20 membered polycyclic hydrocarbon group, wherein each ring in the system shares an adjacent pair of carbon atoms with another ring, wherein one or more rings can contain one or more double bonds, but none of the rings has a completely conjugated pi-electron system. Preferably, a fused cycloalkyl group is 6 to 14 membered, more preferably 7 to 10 membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 5-membered/5-membered, or 5-membered/6-membered bicyclic fused cycloalkyl. Representative examples of fused cycloalkyls include, but are not limited to, the following substituents:

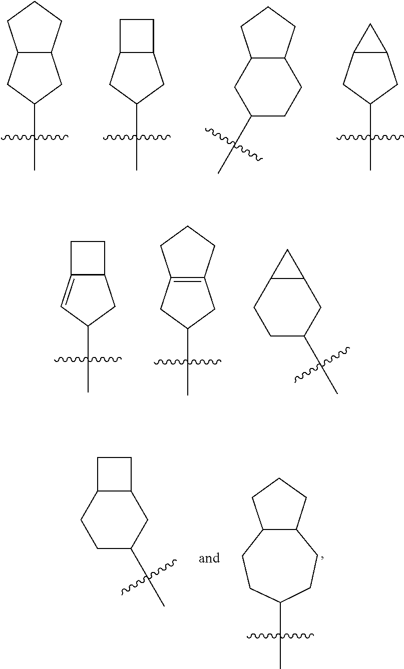

‘Bridged Cycloalkyl’ refers to a 5 to 20 membered polycyclic hydrocarbon group, wherein every two rings in the system share two disconnected carbon atoms. The rings can have one or more double bonds, but have no completely conjugated pi-electron system. Preferably, a bridged cycloalkyl is 6 to 14 membered, and more preferably 7 to 10 membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following substituents:

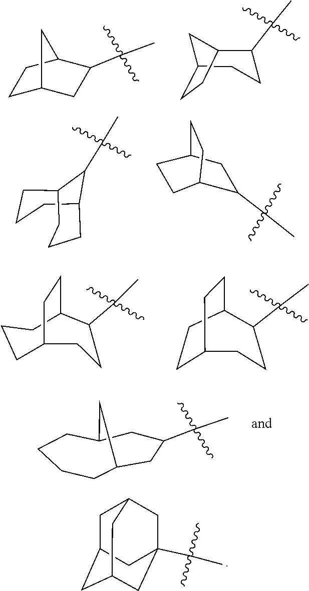

The cycloalkyl can be fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably one to five, sometimes more preferably one to three, substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group. ‘Heterocyclyl’ refers to a 3 to 20 membered saturated and/or partially unsaturated monocyclic or polycyclic hydrocarbon group having one or more, sometimes preferably one to five, sometimes more preferably one to three, heteroatoms selected from the group consisting of N, O, and S(O)ₘ (wherein m is 0, 1, or 2) as ring atoms, but excluding —O—O—, —O—S— or —S—S— in the ring, the remaining ring atoms being C. Preferably, heterocyclyl is a 3 to 12 membered having 1 to 4 heteroatoms; more preferably a 3 to 10 membered having 1 to 3 heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring. ‘Spiro heterocyclyl’ refers to a 5 to 20 membered polycyclic heterocyclyl with rings connected through one common carbon atom (called a spiro atom), wherein said rings have one or more, sometimes preferably one to five, sometimes more preferably one to three, heteroatoms selected from the group consisting of N, O, and S(O)ₘ (wherein m is 0.1 or 2) as ring atoms, the remaining ring atoms being C, wherein one or more rings can contain one or more double bonds, but none of the rings has a completely conjugated pi-electron system. Preferably a spiro heterocyclyl is 6 to 14 membered, and more preferably 7 to 10 membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following substituents:

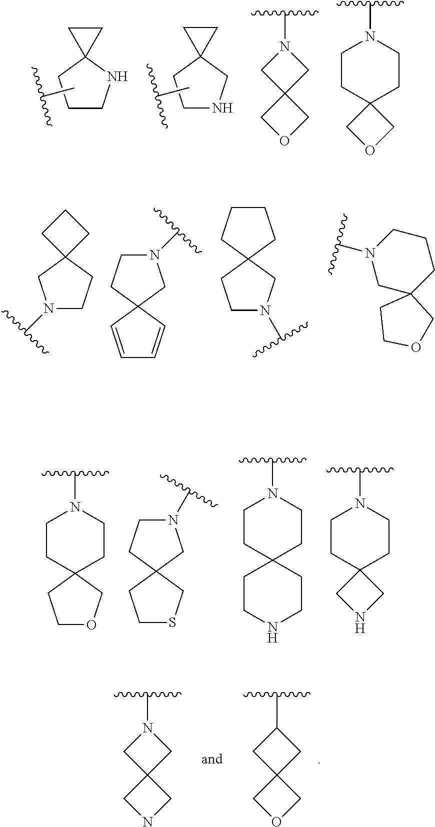

‘Fused Heterocyclyl’ refers to a 5 to 20 membered polycyclic heterocyclyl group, wherein each ring in the system shares an adjacent pair of carbon atoms with the other ring, wherein one or more rings can contain one or more double bonds, but none of the rings has a completely conjugated pi-electron system, and wherein said rings have one or more, sometimes preferably one to five, sometimes more preferably one to three, heteroatoms selected from the group consisting of N, O, and S(O)ₚ (wherein p is 0, 1, or 2) as ring atoms, the remaining ring atoms being C. Preferably a fused heterocyclyl is 6 to 14 membered, and more preferably 7 to 10 membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 5-membered/5-membered, or 5-membered/6-membered bicyclic fused heterocyclyl. Representative examples of fused heterocyclyl include, but are not limited to, the following substituents:

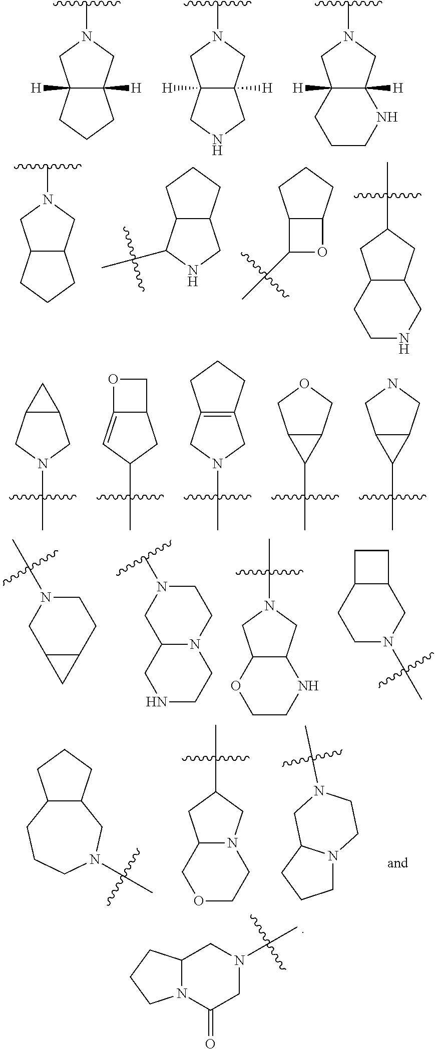

‘Bridged Heterocyclyl’ refers to a 5 to 14 membered polycyclic heterocyclic alkyl group, wherein every two rings in the system share two disconnected atoms, the rings can have one or more double bonds, but have no completely conjugated pi-electron system, and the rings have one or more heteroatoms selected from the group consisting of N, O, and S(O)ₘ (wherein m is 0, 1, or 2) as ring atoms, the remaining ring atoms being C. Preferably a bridged heterocyclyl is 6 to 14 membered, and more preferably 7 to 10 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following substituents:

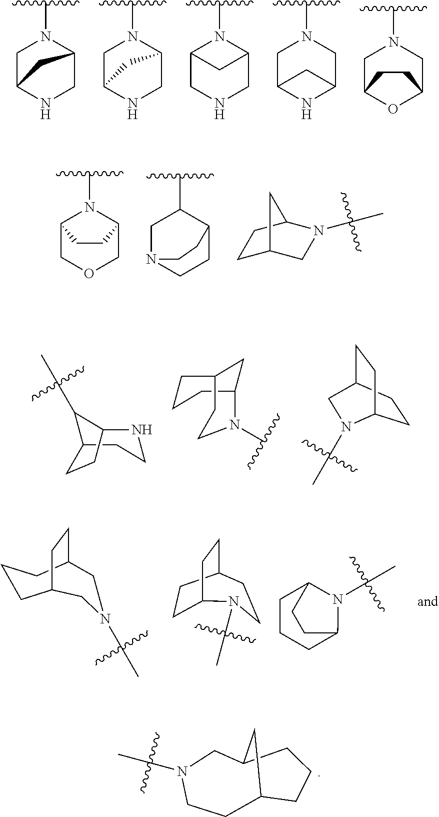

The ring of said heterocyclyl can be fused to the ring of an aryl, heteroaryl or cycloalkyl, wherein the ring bound to the parent structure is heterocyclyl. Representative examples include, but are not limited to the following substituents:

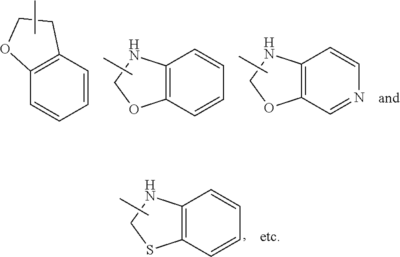

The heterocyclyl is optionally substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio. ‘Heterocycle’ is used, it is intended to include heteroaryl.

‘Pharmaceutical composition’ refers to a mixture of one or more of the compounds described in the present invention or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

‘Pharmaceutically acceptable salts’ refer to salts of the compounds of the invention, such salts being safe and effective when used in a mammal and have corresponding biological activity.

‘Optional’ or ‘optionally’ means that the event or circumstance described subsequently can, but need not, occur, and the description includes the instances in which the event or circumstance may or may not occur. For example, ‘the heterocyclic group optionally substituted by an alkyl’ means that an alkyl group can be, but need not be, present, and the description includes the case of the heterocyclic group being substituted with an alkyl and the heterocyclic group being not substituted with an alkyl.

‘Substituted’ refers to one or more hydrogen atoms in the group, preferably up to 5, more preferably 1 to 3 hydrogen atoms, independently substituted with a corresponding number of substituents. It goes without saying that the substituents exist in their only possible chemical position. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

‘Conjugate’ as used herein refers to a biomolecule which is covalently linked to a non-nucleotide moiety. Specific conjugates of the invention are nucleic acid conjugates where a nucleic acid molecule, e.g. an oligonucleotide or siRNA, is covalently linked to a non-nucleotide moiety, such as a GalNAc conjugate moiety of the present invention.

‘Oligonucleotide’ as used herein is defined as it is generally understood by the skilled person as a molecule comprising two or more covalently linked nucleosides. Such covalently bound nucleosides may also be referred to as nucleic acid molecules or oligomers.

Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

‘siRNA’ may be composed of two annealed polynucleotides or a single polynucleotide that forms a hairpin structure. An siRNA molecule of the invention comprises a sense region and an antisense region. The antisense region having a nucleotide sequence identical (perfectly complementary) or nearly identical (partially complementary) to a target nucleic acid.

RNAi molecules is essentially a synthetic, man-made molecule, and operates within the RNA interference (RNAi) pathway, where it interferes with a target nucleic acid with complementary nucleotide sequences by degrading mRNA after transcription, resulting in no translation. The siRNA interacts via a protein complex called Dicer, which dices up siRNA into smaller fragments. One strand of these fragments, in most cases the antisense strand, is loaded into another protein complex called the RNA-induced Silencing Complex (RISC). The strand bound by RISC then links the complex to the target nucleic acid by base pairing. The target nucleic acid is cleaved and destroyed so no protein can be synthesized.

‘Inhibiting expression’ refers to a reduction or blockade of the expression or activity and does not necessarily indicate a total elimination of expression or activity.

### EXAMPLES

The following examples serve to illustrate the invention, but the examples should not be considered as limiting the scope of the invention. If specific conditions for the experimental method are not specified in the examples of the present invention, they are generally in accordance with conventional conditions or recommended conditions of the raw materials and the product manufacturer. The reagents without a specific source indicated are commercially available, conventional reagents.

The structure of each compound is identified by nuclear magnetic resonance (NMR) and/or mass spectrometry (MS). NMR chemical shifts (8) are given in 10⁻⁶ (ppm). NMR is determined by Varian Mercury 300 MHz Bruker Avance III 400 MHZ machine. The solvents used are deuterated-dimethyl sulfoxide (DMSO-d₆), deuterated-chloroform (CDCl₃) and deuterated-methanol (CD₃OD).

High performance liquid chromatography (HPLC) is determined on an Agilent 1200DAD high pressure liquid chromatography spectrometer (Sunfire C18 150×4.6 mm chromatographic column) and a Waters 2695-2996 high pressure liquid chromatography spectrometer (Gimini C18 150×4.6 mm chromatographic column). Liquid Chromatography Mass Spectrometry (LCMS) is determined on an Agilent 1200 high pressure liquid chromatography spectrometer & mass spectrometry (Sunfire C18 4.6*50 mm 3.5 um chromatographic column) and an Agilent 19091S-433 HP-5 high pressure liquid chromatography spectrometer & mass spectrometry (XBridge C18 4.6*50 mm 3.5 um chromatographic column).

Chiral High performance liquid chromatography (HPLC) is determined on SFC Thar 80 & 150 & 200 (waters.)

The thin-layer silica gel plates used in thin-layer chromatography are Yantai Xinnuo silica gel plate. The dimension of the plates used in TLC is 0.15 mm to 0.2 mm, and the dimension of the plates used in thin-layer chromatography for product purification was 0.4 mm to 0.5 mm.

Column chromatography generally uses Qingdao Haiyang 200 to 300 mesh silica gel as carrier.

The known starting material of the invention can be prepared by the conventional synthesis method in the prior art, or can be purchased from ABCR GmbH & Co. KG, Acros Organics, Aldrich Chemical Company, Accela ChemBio Inc or Dari chemical Company, etc.

Unless otherwise stated in the examples, the following reactions are placed under argon atmosphere or nitrogen atmosphere.

The term “argon atmosphere” or “nitrogen atmosphere” means that a reaction flask is equipped with a balloon having 1 L of argon or nitrogen.

The term “hydrogen atmosphere” means that a reaction flask was equipped with a balloon having 1 L of hydrogen.

MS is mass spectroscopy with (+) referring to the positive mode which generally gives a M+1 (or M+H) absorption where M=the molecular mass.

### Example 1

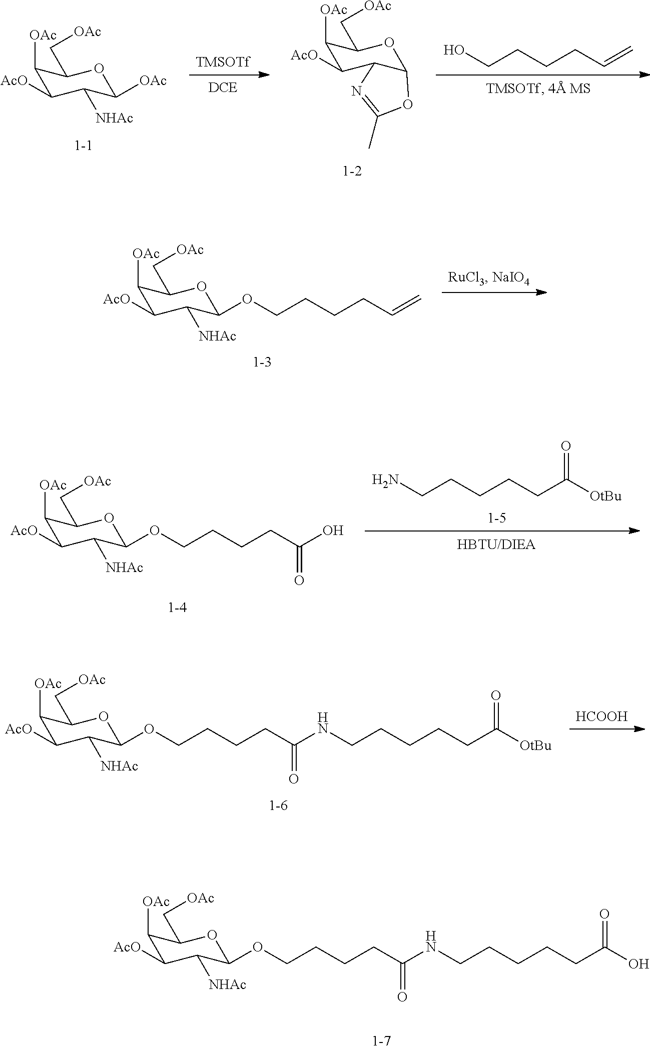

### Compound 1-2:

Compound 1-1 (5 g, 12.84 mmol) in anhydrous 1,2-dichloroethane (30 mL) was stirred at 0° C., TMSOTf (3.43 g, 15.41 mmol, 2.78 mL) was added dropwise under constant stirring over 10 min, and the stirring was continued overnight at room temperature. The reaction mixture was quenched with cold saturated NaHCO₃ solution (200 mL), and the organic layer was separated. The product was extracted with dichloromethane (60 mL×2); the combined organic layers were washed with water and brine, dried over anhydrous Na₂SO₄, and evaporated to dryness under reduced pressure to obtain compound 1-2 (4.23 g, 99% yield) as a yellow oil, which was used without further purification.

Mass calc. for C₁₄H₁₉NO₈: 329.1; found: 330.1 [M+H]⁺, ESI.

### Compound 1-3:

Compound 1-2 (4.23 g, 12.85 mmol) in anhydrous 1,2-dichloroethane (20 mL) was stirred with 4 Å molecular sieves (4.7 g) for 5 min at room temperature. 5-Hexen-1-ol (1.42 g, 14.13 mmol) was added and stirring was continued for 30 min. TMSOTf (1.43 g, 6.42 mmol, 1.16 mL) was added dropwise at 0° C. and the stirring was continued for 2 h at room temperature. The reaction mixture was quenched with cold saturated NaHCO₃ solution (100 mL), and the organic layer was separated. The product was extracted with dichloromethane (60 mL×2); the combined organic layers were washed with water, dried over anhydrous Na₂SO₄, and evaporated to dryness under reduced pressure to obtain compound 1-3 (5.5 g, 99% yield) as a yellow oil, which was used without further purification.

Mass calc. for C₂₀H₃₁NO₉: 429.2; found: 430.2 [M+H]⁺, ESI.

### Compound 1-4:

To a solution of compound 1-3 (5.5 g, 12.81 mmol) in DCM (35 mL) and MeCN (35 mL) was added 4.0 mol equiv. of sodium (meta) periodate (10.96 g, 51.24 mmol) in water (45.5 mL). The mixture was cooled to 0° C. in an ice bath and stirred for 15 min. Ruthenium chloride trihydrate (110.5 mg, 423 μmol) was added to the cold reaction mixture. The reaction mixture was stirred at room temperature for 4 h. The reaction mixture was diluted with water (90 mL), and the pH was adjusted to 7.5 by adding solid NaHCO₃. The DCM layer was removed, the aqueous layer was washed with DCM (30 mL×2), and organic extracts were discarded. pH of the aqueous layer was adjusted to 3 by addition of citric acid, and the carboxylic acid 1-4 was extracted into DCM (50 mL×3). The organic layer was stirred with saturated brine (50 mL×1) followed by drop-wise addition of Na₂S₂O₃ solution (50 mL×1) until the dark green organic phase turned to a pale-yellow color. The layers were separated, and the organic layer was dried over anhydrous Na₂SO₄ and evaporated under reduced pressure to afford compound 1-4 as a white solid (2.3 g, 40% yield). which was used without further purification.

Mass calc. for C₁₉H₂₉NO₁₁: 447.2; found: 448.2 [M+H]⁺, ESI.

¹H NMR (400 MHZ, DMSO-d₆) δ 7.81 (d, J=9.2 Hz, 1H), 5.84-5.73 (m, 1H), 5.21 (d, J=3.3 Hz, 1H), 4.98-4.93 (m, 3H), 4.48 (d, J=8.5 Hz, 1H), 4.04-4.00 (m, 3H), 3.88-3.83 (m, 1H), 3.73-3.68 (m, 1H), 3.40-3.36 (m, 1H), 2.10 (s, 3H), 2.03-2.02 (m, 2H), 2.00 (s, 3H), 1.89 (s, 3H), 1.76 (s, 3H), 1.43-1.32 (m, 4H).

### Compound 1-6:

To a solution of compound 1-5 (1.16 g, 6.17 mmol) and the acid 1-4 (2.3 g, 5.14 mmol) in DMF (30 mL) were added HBTU (2.92 g, 7.71 mmol) and DIPEA (1.99 g, 15.42 mmol, 2.69 mL). The reaction was stirred for 43 h at room temperature and diluted with water (150 mL). The mixture was extracted with ethyl acetate (60 mL×3). The combined organic layers were washed consecutively with water (100 mL×3) and brine (100 mL). After drying over anhydrous Na₂SO₄, the solvent was evaporated under reduced pressure to obtain the compound 1-6 as a yellow oil (4.3 g, crude), which was used without further purification.

Mass calc. for C₂₉H₄₈N₂O₁₂: 616.3; found: 617.4 [M+H]⁺, ESI.

### Compound 1-7:

Compound 1-6 (4.23 g, assuming 6.86 mmol) was added to formic acid (30 mL), the mixture was stirred at room temperature overnight. The reaction was completed determined by LC-MS. The mixture was then evaporated under reduced pressure and purified by silica gel chromatography (DCM:MeOH=10:1) to obtain the compound 1-7 as a yellow oil (2.88 g, 75% yield over 2 steps).

Mass calc. for C₂₅H₄₀N₂O₁₂: 560.3; found: 561.3 [M+H]⁺, ESI.

¹H NMR (400 MHZ, DMSO-d₆) δ12.02 (s, 1H), 7.83 (d, J=9.2 Hz, 1H), 7.25 (t, J=5.6 Hz, 1H), 5.21 (d, J=3.2 Hz, 1H), 4.96 (dd, J=11.2, 3.6 Hz, 1H), 4.47 (d, J=8.4 Hz, 1H), 4.04-4.00 (m, 3H), 3.90-3.83 (m, 1H), 3.73-3.68 (m, 1H), 3.42-3.36 (m, 1H), 3.02-2.97 (m, 1H), 2.18 (t, J=7.2 Hz, 2H), 2.10 (s, 3H), 2.02 (t, J=7.2 Hz, 2H), 1.99 (s, 3H), 1.89 (s, 3H), 1.77 (s, 3H), 1.54-1.41 (m, 7H), 1.27-1.21 (m, 3H).

### Example 2

### Method 1:

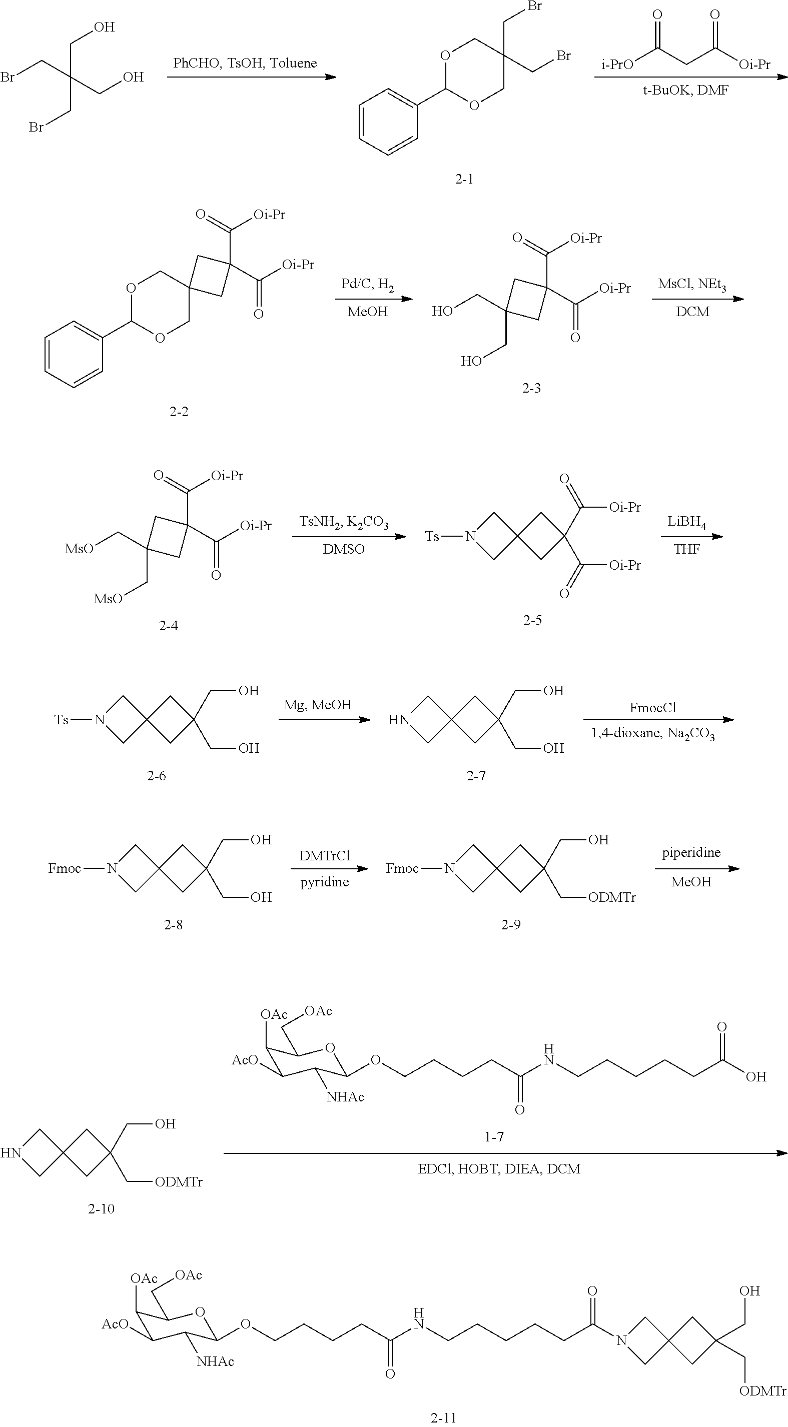

### Compound 2-1:

A mixture of 2,2-bis(bromomethyl) propane-1,3-diol (270 g, 1.03 mol), benzaldehyde (114.86 g, 1.08 mol), TsOH (17.74 g, 340.55 mmol) in toluene (1 L) was refluxed for 6 h. The mixture was cooled and extracted with EA (1 L), washed with NaHCO₃ solution, then washed with brine, dried over Na₂SO₄. The solvent was removed under reduced pressure, and the residue was recrystallized by MeOH to give desired product 2-1 (247 g, 68% yield).

¹H NMR (400 MHZ, DMSO-d₆) δ 7.47-7.41 (m, 2H), 7.40-7.36 (m, 3H), 5.48 (s, 1H), 4.09-3.93 (m, 6H), 3.46 (s, 2H).

### Compound 2-2:

To a suspension of t-BuOK (137.27 g, 1.43 mol) in dry DMF (700 mL), diisopropyl malonate (268.85 g, 1.43 mol) was added dropwise (the temperature being maintained below 70° C.) followed by adding compound 2-1 (250 g, 714.19 mmol). The resulting reaction mixture was heated at 140° C. for 6 h. After cooled, saturated NH₄Cl solution (1.5 L) was added, and the mixture was extracted with hexanes (500 mL×3). The combined organic extracts were dried over sodium sulfate and concentrated in vacuo. A solid product was separated from the liquid residue by filtration, washed with hexane (100 mL×2) and dried to yield pure product 2-2 as a white solid (194 g, 68% yield).

Mass calc. for C₂₁H₂₈O₆: 376.2; found: 377.2 [M+H]⁺, ESI.

¹H NMR (400 MHZ, DMSO-d₆) δ 7.45-7.31 (m, 5H), 5.45 (s, 1H), 5.02-4.93 (m, 2H), 3.96 (d, J=11.2 Hz, 2H), 3.77 (d, J=11.1 Hz, 2H), 2.55 (s, 2H), 2.13 (s, 2H), 1.23-1.15 (m, 12H).

### Compound 2-3:

To a solution of compound 2-2 (155 g, 411.7 mmol) in MeOH (750 mL), 10% Pd/C (31 g) was added, and the resulting suspension was hydrogenated under 5 atm H2 at ambient temperature upon stirring for 48 h. Filtration of the catalyst and removal of the solvent in vacuo was to yield compound 2-3 as a colorless oil (118 g, 99% yield), which was used in the next step without further purification.

Mass calc. for C₂₁H₂₈O₆: 288.2; found: 289.4 [M+H]⁺, ESI.

### Compound 2-4:

To a solution of compound 2-3 (140 g, 485.5 mmol) in dichloromethane (840 mL), mesyl chloride (155.7 g, 1.36 mol) was added. The resulting mixture was cooled to −30° C., then triethylamine (323.9 g, 3.2 mol) was added dropwise. After the addition was complete, the reaction mixture was warmed to ambient temperature and stirred for 12 h, then washed with water (1000 mL), 10% aqueous citric acid (1000 mL) and brine (1000 mL). The organic phase was dried over sodium sulfate and evaporated at reduced pressure to give compound 2-4 (125 g, 58% yield).

Mass calc. for C₂₁H₂₈O₆: 444.1; found: 445.2 [M+H]⁺, ESI.

¹H NMR (400 MHZ, DMSO-d₆) δ 5.02-4.93 (m, 2H), 4.20 (s, 4H), 3.23 (s, 6H), 2.45 (s, 4H), 1.22-1.14 (m, 12H).

### Compound 2-5:

A solution of compound 2-4 (63 g, 141.73 mmol), potassium carbonate (100.88 g, 729.89 mmol), and p-toluenesulfonamide (25.48 g, 148.81 mmol) in DMSO (300 mL) was heated at 85° C. for 12 h. After cooled, water (300 mL) was added, and the mixture was extracted with EtOAc (600 mL). The combined organic phases were washed with 10% aqueous citric acid (600 mL) and brine (600 mL), dried over sodium sulfate and evaporated in vacuo, then recrystallization with isopropyl ether to yield compound 2-5 (48 g, 80% yield).

Mass calc. for C₂₁H₂₈O₆ 423.2; found: 424.2 [M+H]⁺, ESI.

¹H NMR (400 MHZ, DMSO-d₆) δ 7.67 (d, J=8.2 Hz, 2H), 7.45 (d, J=8.0 Hz, 2H), 4.45 (t, J=5.4 Hz, 2H), 3.60 (s, 4H), 3.14 (d, J=5.7 Hz, 4H), 2.43 (s, 3H), 1.67 (s, 4H).

### Compound 2-6:

To a solution of compound 2-5 (58.7 g, 138.60 mmol) in THF (100 mL), a solution of lithium borohydride (2 M) in THF (263.82 mL) was added at −20° C. The resulting mixture was stirred at room temperature for 16 h. The mixture was slowly added to ice water (1 L). pH was adjusted to 7 with citric acid aqueous solution. The mixture was extracted with EA (500 mL), dried with anhydrous sodium sulfate and evaporated at reduced pressure to give compound 2-6 (40 g, 93% yield).

Mass calc. for C₂₁H₂₈O₆: 311.1; found: 312.1 [M+H]⁺, ESI.

¹H NMR (400 MHZ, DMSO-D6) δ 7.67 (d, J=8.2 Hz, 2H), 7.45 (d, J=8.0 Hz, 2H), 4.45 (t, J=5.4 Hz, 2H), 3.60 (s, 4H), 3.14 (d, J=5.7 Hz, 4H), 2.43 (s, 3H), 1.67 (s, 4H).

### Compound 2-7:

Compound 2-6 (5 g, 16.1 mmol) and Mg (powder, 3.47 g, 144.6 mmol) were mixed in MeOH (anhydrous, 60 mL) and stirred overnight at rt. LC-MS suggested full conversion, and water (30 mL) was added. A white precipitate formed. The mixture was filtered, and the filtrate was adjusted to pH 6 by HCl (aq. 4M), and concentrated to dryness to give compound 2-7 (2.55 g crude) as a yellow solid. The crude product was used without further purification.

Mass calc. for C₈H₁₅NO₂: 157.1; found: 158.1 [M+H]⁺, ESI.

### Compound 2-8:

Compound 2-7 (2.55 g, assuming 16.2 mmol) was dissolved in dioxane (20 mL), and FmocCl (4.60 g, 17.8 mmol) was added slowly at 0° C., followed by Na₂CO₃ (saturated, 20 mL). The reaction was stirred at 30° C. for 3 h. LC-MS suggested full conversion. The reaction mixture was extracted by EA (20 mL×3). The organic phase was concentrated and purified by flash column chromatography on silica gel (DCM:MeOH=97:3) to give a white solid 2-8 (2.75 g, 44.7% yield over 2 steps).

Mass calc. for C₂₃H₂₅NO₄: 379.2; found: 380.2 [M+H]⁺, ESI.

### Compound 2-9:

Compound 2-8 (2.7 g, 7.1 mmol) and pyridine (20 mL) were mixed in a flask and DMTr-Cl (2.4 g, 7.1 mmol) was added batch-wise. The reaction was stirred at rt for 4 h. LC-MS suggested full conversion. The reaction mixture was extracted by EA (20 mL×3). The organic phase was concentrated and purified by flash column chromatography on silica gel (DCM:MeOH=97:3) to give a yellow solid 2-9 (2.8 g, 58% yield).

Mass calc. for C₄₄H₄₃NO₆: 681.3; found: 682.3 [M+H]⁺, ESI.

### Compound 2-10:

Compound 2-9 (2.3 g, 6.8 mmol), piperidine (5 mL) and MeOH (anhydrous, 45 mL) were mixed in a flask and stirred at 30° C. for 3 h. LC-MS suggested full conversion. The reaction mixture was concentrated and purified by flash column chromatography on silica gel (DCM:MeOH=95:5) to give a yellow solid 2-10 (0.5 g, 33% yield).

Mass calc. for C₂₉H₃₃NO₄: 459.2; found: 460.2 [M+H]⁺, ESI.

### Compound 2-11:

Compound 1-7 (325 mg, 0.58 mmol), HOBT (105 mg, 0.77 mmol) and EDCI (150 mg, 0.78 mmol) were dissolved in DCM (6 mL) and stirred at rt for 15 min, then cooled to 0° C. DIPEA (211 mg, 1.6 mmol) was added followed by compound 2-9 (300 mg, 0.65 mmol), and stirred at rt for 4 h. LC-MS suggested full conversion. NaHCO₃ (saturated, 10 mL) was added. The reaction mixture was extracted by DCM (5 mL×3). The organic phase was concentrated and purified by flash column chromatograph on silica gel (DCM:MeOH=95:5) to give crude compound 2-11 as a white solid (350 mg, 88% purity at 210 nm, 46% yield). Purification by prep-HPLC: crude compound 2-7 (1.8 g, 88% purity at 210 nm) was purified by prep-HPLC (C-18 column, water/ACN, 10%-80% ACN) to give compound 2-11 (650 mg, 42% recovery yield). Mass calc. for C₅₄H₇₁N₃O₁₅: 1001.5; found: 1024.5 [M+Na]⁺, ESI.

¹H NMR (400 MHZ, DMSO) δ 7.82 (d, J=9.2 Hz, 1H), 7.75-7.69 (br, 1H), 7.40-7.35 (m, 2H), 7.37-7.21 (m, 7H), 6.95-6.88 (m, 4H), 5.21 (d, J=3.3 Hz, 1H), 5.00-4.90 (m, 1H), 4.66-4.63 (m, 1H), 4.48 (d, J=8.4 Hz, 1H), 4.08-4.00 (br, 4H), 3.91-3.83 (m, 1H), 3.73 (s, 6H), 3.72-3.66 (m, 3H), 3.51 (s, 1H), 3.43-3.37 (m, 3H), 3.03-2.96 (m, 2H), 2.91 (s, 2H), 2.10 (s, 3H), 2.00-1.76 (m, 17H), 1.49-1.30 (m, 8H), 1.23-1.19 (m, 2H).

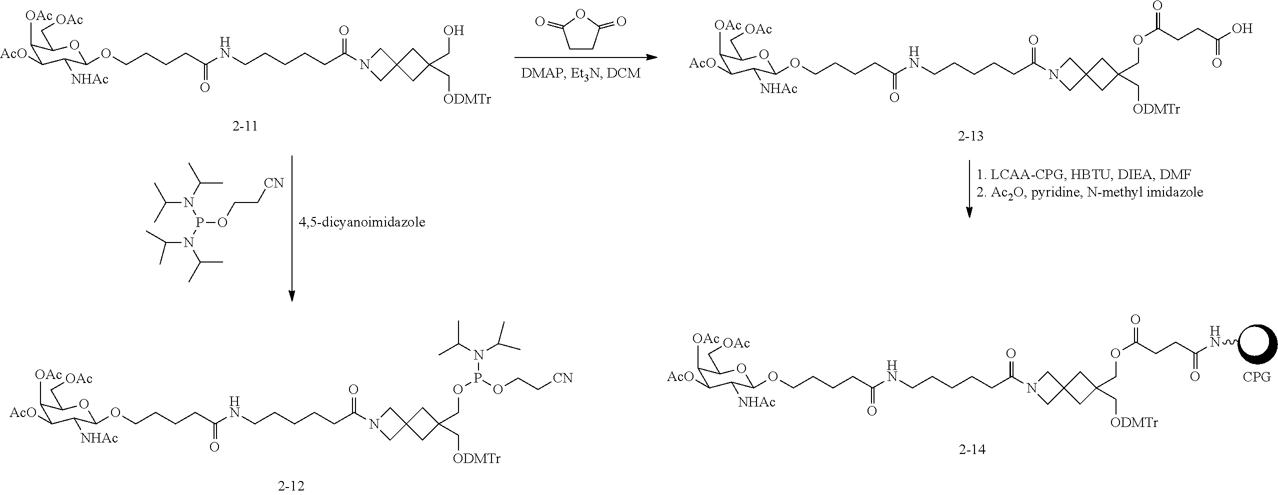

### Compound 2-12:

To a solution of compound 2-11 (300.0 mg, 0.30 mmol) in anhydrous DCM (3.0 mL) was added 4,5-dicyanoimidazole (32.0 mg, 0.27 mmol) and 2-Cyanoethyl N,N,N′,N′-tetraisopropylphosphorodiamidite (108 mg, 0.36 mmol) at rt and stirred for 1 h. LCMS showed the starting material was consumed completely. The solution was quenched by water (100 mL), washed with brine (300 mL×3) and dried over by Na₂SO₄. Then the solution was concentrated under reduced pressure and the residue was purified by Flash-Prep-HPLC with the following conditions (Column: C18 silica gel; mobile phase: CH₃CN/H₂O=1/1 increasing to CH₃CN/H₂O=1/0 within 20 min; Detector: UV 254 nm). This resulted in giving compound 2-12 as a white solid (177 mg, 49% yield).

Mass calc. for C₆₃H₈₈N₅O₁₆P: 1201.60; found: 1202.6 [M+H]⁺, ESI.

¹HNMR (600 MHZ, CD₃CN) δ 7.37-7.34 (m, 2H), 7.24-7.20 (m, 6H), 7.14-7.12 (m, 1H), 6.79-6.77 (m, 4H), 6.57-6.54 (m, 1H), 6.44-6.43 (m, 1H), 5.20 (d, J=6.0 Hz, 1H), 4.93-4.91 (m, 1H), 4.44 (d, J=6.0 Hz, 1H), 4.04-3.83 (m, 5H), 3.73-3.39 (m, 16H), 3.04-2.91 (m, 4H), 2.52-2.50 (m, 2H), 2.03-1.75 (m, 24H), 1.53-1.33 (m, 9H), 1.21-1.04 (m, 15H).

³¹PNMR (242 MHZ, CD₃CN) δ 147.37, 147.30.

### Compound 2-13:

To a solution of compound 2-11 (80 mg, 0.080 mmol) in anhydrous DCM (1.0 mL), DMAP (5 mg, 0.04 mmol) and TEA (24 mg, 0.24 mmol) were added, followed by adding succinic anhydride (20 mg, 0.2 mmol). The reaction mixture was stirred at rt for 3 h, LCMS showed the starting material was consumed completely. The reaction mixture was diluted with DCM (10 mL) and washed with H₂O (3 mL×4), then washed with brine (3 mL×4), the organic layer was concentrated to give compound 2-13 as a white solid (85 mg, 97% yield).

Mass calc. for C₅₈H₇₅N₃O₁₈: 1101.50; found: 1100.4 [M−H], ESI.

¹HNMR (600 MHZ, DMSO-d₆) δ 12.22 (s, 1H), 7.84-7.81 (m, 1H), 7.71-7.69 (m, 1H), 7.37-7.16 (m, 9H), 6.92-6.89 (m, 4H), 5.21 (d, J=6.0 Hz, 1H), 4.97-4.95 (m, 1H), 4.48 (d, J=12.0 Hz, 1H), 4.07-4.01 (m, 6H), 3.89-3.84 (m, 1H), 3.73-3.65 (m, 9H), 3.49 (s, 1H), 3.41-3.38 (m, 1H), 3.01-2.95 (m, 4H), 2.45-2.44 (m, 4H), 2.09-1.76 (m, 20H), 1.48-1.19 (m, 10H).

### Solid Support 2-14:

Native amino-LCAA-CPG (loading value: 75 umol/g, 1000 Å) was washed with ACN (100 mL×2), DMF (100 mL×2) and DCM (100 mL×2), then dried under high vacuum overnight.

To a solution of succinate 2-13 (85 mg, 0.077 mmol) and HBTU (53 mg, 0.14 mmol) in anhydrous DMF (1.5 mL), DIPEA (30 mg, 0.23 mmol) was added, the reaction mixture was shaken at RT for 10 min, then native amino-LCAA-CPG (250 mg, loading 75 umol/g) was added, the suspension was shaken at rt for 20 h, then filtered, washed with DMF (20 mL×5), ACN (20 mL×5), DCM (20 mL×5) until TLC showed no spot at 254 nm in eluent. The solid support was dried under vacuum for 2 h to give solid support (260 mg). The unreacted amines on the solid support were capped by stirring with Ac2O/pyridine/N-methyl imidazole (90 μL/1.0 mL/80 μL) at RT for 1 h, washed with DMF (20 mL×5), CAN (20 mL×5), DCM (20 mL×5) until TLC showed no spot at 254 nm in eluent. The solid support was dried under vacuum for 15 h to give solid support 2-14 (260 mg). For loading calculation, take 6.5 mg of the dried loaded CPG and add 25 mL of 3% DCA in DCM. Shake the solution and measure UV absorbance at 500 nm. Make sure the absorbance value is below 1.0 unit to ensure no signal saturation. Then apply the following equation: Loading in μmol/g=(total volume of DCA added in mL)*(Abs value at 500 nm)*1000)/(76*(mg of CPG taken))
Total volume of DCA added in mL=25 mL
Abs value at 500 nm=0.7721
mg of CPG taken=6.5 mg
Loading in μmol/g=((25)*(0.7721)*1000)/(76*(6.5))=39 μmol/g.

### Method 2:

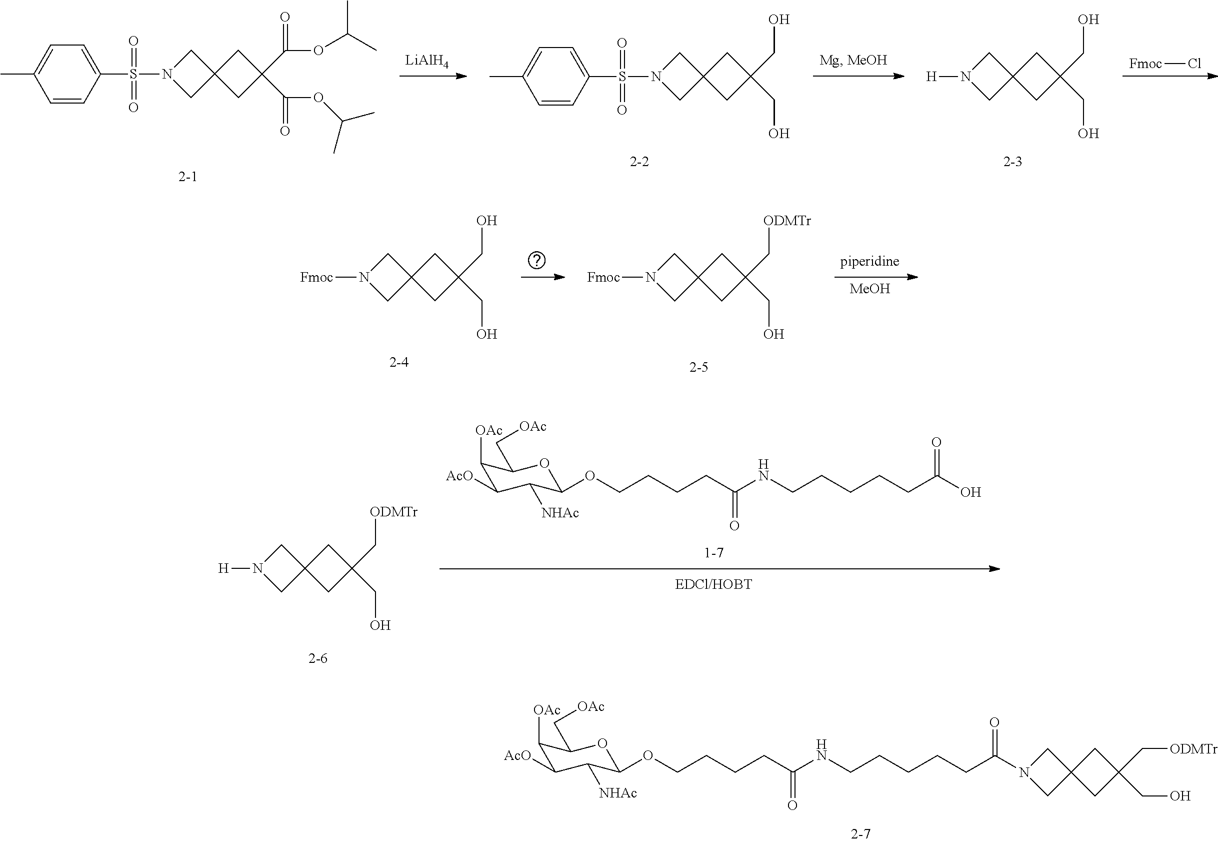

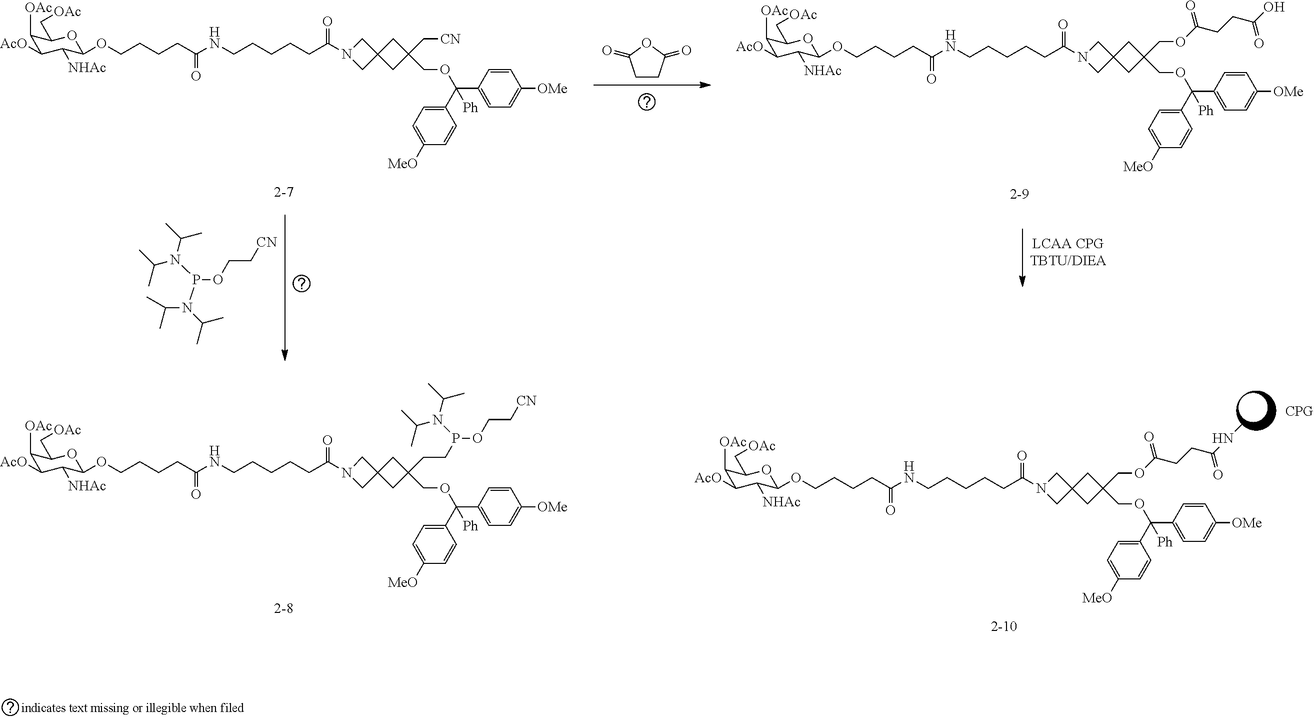

### Compound 2-2:

Compound 2-1 (11 g, 26.0 mmol) was dissolved in THF (anhydrous, 100 mL) in 250 mL flask. LiAlH₄ (1.97 g, 52 mmol) was added batch-wise under 0° C. over 15 min. The reaction was then stirred at rt for 4 h. LC-MS suggested full conversion. The reaction was cooled to 0° C., and water (2 mL) was added slowly, followed by NaOH (10%, 2 mL), then water (6 mL). The mixture was filtered, and the filtrate was concentrated to dryness to give compound 2-2 (6.5 g, 80% yield) as a white solid. The crude product was used without further purification. C₁₅H₂₁NO₄S for: 311.1; found: 312.1 [M+H]⁺, ESI.

¹H NMR (400 MHZ, DMSO) δ 7.67 (d, J=8.0 Hz, 2H), 7.48 (d, J=8.0 Hz, 2H), 4.46 (t, J=5.4 Hz, 2H), 3.60 (s, 4H), 3.31 (d, J=5.6 Hz, 4H), 2.43 (s, 3H), 1.67 (s, 4H).

### Compound 2-3:

Compound 2-2 (5 g, 16.1 mmol) and Mg (powder, 3.47 g, 144.6 mmol) were mixed in MeOH (anhydrous, 60 mL) and stirred overnight at rt. LC-MS suggested full conversion, and water (30 mL) was added. A white precipitate formed. The mixture was filtered, and the filtrate was adjusted to pH 6 by HCl (aq. 4M), and concentrated to dryness to give compound 2-3 (2.55 g crude) as a yellow solid. The crude product was used without further purification.

Mass calc. C₈H₁₅NO₂ for: 157.1; found: 158.1 [M+H]⁺, ESI.

### Compound 2-4:

Compound 2-3 (2.55 g, assuming 16.2 mmol) was dissolved in dioxane (20 mL), and FmocCl (4.60 g, 17.8 mmol) was added slowly at 0° C., followed by Na₂CO₃ (saturated, 20 mL). The reaction was stirred at 30° C. for 3 h. LC-MS suggested full conversion. The reaction mixture was extracted by EA (20 mL×3). The organic phase was concentrated and purified by flash column chromatography on silica gel (DCM:MeOH=97:3) to give a white solid 2-4 (2.75 g, 44.7% yield, 2 steps).

Mass calc. for C₂₃H₂₅NO₄: 379.2; found: 380.2 [M+H]⁺, ESI.

### Compound 2-5:

Compound 2-4 (2.7 g, 7.1 mmol) and pyridine (20 mL) were mixed in a flask and DMTr-Cl (2.4 g, 7.1 mmol) was added batch-wise. The reaction was stirred at rt for 4 h. LC-MS suggested full conversion. The reaction mixture was extracted by EA (20 mL×3). The organic phase was concentrated and purified by flash column chromatography on silica gel (DCM:MeOH=97:3) to give a yellow solid 2-5 (2.8 g, 58% yield).

Mass calc. for C₄₄H₄₃NO₆: 681.3; found: 682.3 [M+H]⁺, ESI.

### Compound 2-6:

Compound 2-5 (2.3 g, 6.8 mmol), piperidine (5 mL) and MeOH (anhydrous, 45 mL) were mixed in a flask and stirred at 30° C. for 3 h. LC-MS suggested full conversion. The reaction mixture was concentrated and purified by flash column chromatography on silica gel (DCM:MeOH=95:5) to give a yellow solid 2-6 (0.5 g, 33% yield).

Mass calc. for C₂₉H₃₃NO₄: 459.2; found: 460.2 [M+H]⁺, ESI.

### Compound 2-7:

Compound 1-7 (325 mg, 0.58 mmol), HOBT (105 mg, 0.77 mmol) and EDCI (150 mg, 0.78 mmol) were dissolved in DCM (6 mL) and stirred at rt for 15 min, then cooled to 0° C. DIPEA (211 mg, 1.6 mmol) was added followed by compound 2-6 (300 mg, 0.65 mmol), and stirred at rt for 4 h. LC-MS suggested full conversion. NaHCO₃ (saturated, 10 mL) was added. The reaction mixture was extracted by DCM (5 mL×3). The organic phase was concentrated and purified by flash column chromatograph on silica gel (DCM:MeOH=95:5) to give crude compound 2-7 as a white solid (350 mg, 88% purity at 210 nm, 46% yield). Purification by prep-HPLC: crude compound 2-7 (1.8 g, 88% purity at 210 nm) was purified by prep-HPLC (C-18 column, water/ACN, 10%-80% ACN) to give compound 2-7 (650 mg, 96% purity at 210 nm, 42% recovery yield). Mass calc. for C₅₄H₇₁N₃O₁₅: 1001.5; found: 1024.5 [M+Na]⁺, ESI.

¹H NMR (400 MHZ, DMSO) δ 7.82 (d, J=9.2 Hz, 1H), 7.75-7.69 (br, 1H), 7.40-7.35 (m, 2H), 7.37-7.21 (m, 7H), 6.95-6.88 (m, 4H), 5.21 (d, J=3.3 Hz, 1H), 5.00-4.90 (m, 1H), 4.66-4.63 (m, 1H), 4.48 (d, J=8.4 Hz, 1H), 4.08-4.00 (br, 4H), 3.91-3.83 (m, 1H), 3.73 (s, 6H), 3.72-3.66 (m, 3H), 3.51 (s, 1H), 3.43-3.37 (m, 3H), 3.03-2.96 (m, 2H), 2.91 (s, 2H), 2.10 (s, 3H), 2.00-1.76 (m, 17H), 1.49-1.30 (m, 8H), 1.23-1.19 (m, 2H).

### Compound 2-8:

To a solution of compound 2-7 (300.0 mg, 0.30 mmol) in anhydrous DCM (3.0 mL) was added DCI (32.0 mg, 0.27 mmol) and CEP [N(iPr)₂]₂ (108 mg, 0.36 mmol) at rt and stirred for 1 h, LCMS showed SM was consumed completely. The solution was quenched by water (100 mL) washed with brine (300 mL×3) and dry over by Na₂SO₄. Then the solution was concentrated under reduced pressure and the residue was purified by MPLC with the following conditions (Column: C18 silica gel; mobile phase: CH₃CN/H₂O=1/1 increasing to CH₃CN/H₂O=1/0 within 20 min, the eluted product was collected at CH₃CN/H₂O=3/2; Detector: UV 254 nm). This resulted in giving compound 2-8 as a white solid (177 mg, 0.15 mmol, 97.0% purity, 49% yield).

Mass calc. for C₆₃H₈₈N₅O₁₆P: 1201.60; found: 1202.6 [M+H]⁺, ESI.

¹H-NMR (600 MHZ, CD₃CN): 8=7.37-7.34 (m, 2H), 7.24-7.20 (m, 6H), 7.14-7.12 (m, 1H), 6.79-6.77 (m, 4H), 6.57-6.54 (m, 1H), 6.44-6.43 (m, 1H), 5.20-5.19 (d, J=6.0 Hz, 1H), 4.93-4.91 (m, 1H), 4.44-4.43 (d, J=6.0 Hz, 1H), 4.04-3.83 (m, 5H), 3.73-3.39 (m, 16H), 3.04-2.91 (m, 4H), 2.52-2.50 (m, 2H), 2.03-1.75 (m, 24H), 1.53-1.33 (m, 9H), 1.21-1.04 (m, 15H);
³¹PNMR (242 MHZ, CD₃CN) δ 147.37, 147.29.

### Compound 2-9:

To a solution of Compound 2-7 (80 mg, 0.080 mmol) in anhydrous DCM (1.0 mL), DMAP (5 mg, 0.04 mmol) and TEA (24 mg, 0.24 mmol) were added, followed by adding succinic anhydride (20 mg, 0.2 mmol). The reaction mixture was stirred at rt for 3 h, LCMS showed SM was consumed completely. The reaction mixture was diluted with DCM (10 mL) and washed with H₂O (3 mL×4), then washed with brine (3 mL×4), the organic layer was concentrated to give compound 2-9 as a white solid (85 mg, 0.077 mmol, 97% purity, 97% yield) Mass calc. for C₅₈H₇₅N₃O₁₈: 1101.50; found: 1100.4 [M−H], ESI.

¹H-NMR (600 MHZ, DMSO-d₆): δ=12.22 (s, 1H), 7.84-7.81 (m, 1H), 7.71-7.69 (m, 1H), 7.37-7.16 (m, 9H), 6.92-6.89 (m, 4H), 5.21-5.20 (d, J=6.0 Hz, 1H), 4.97-4.95 (m, 1H), 4.49-4.47 (d, J=12.0 Hz, 1H), 4.07-4.01 (m, 6H), 3.89-3.84 (m, 1H), 3.73-3.65 (m, 9H), 3.49 (s, 1H), 3.41-3.38 (m, 1H), 3.01-2.95 (m, 4H), 2.45-2.44 (m, 4H), 2.09-1.76 (m, 20H), 1.48-1.19 (m, 10H).

### Solid Support 2-10:

Native amino-LCAA-CPG (Loading value: 75 umol/g, 1000 Å) was washed with ACN (100 mL×2), DMF (100 mL×2) and DCM (100 mL×2), then dried under high vacuum overnight.

To a solution of succinate 2-9 (85 mg, 0.077 mmol) and HBTU (53 mg, 0.14 mmol) in anhydrous DMF (1.5 mL), DIPEA (30 mg, 0.23 mmol) was added, the reaction mixture was shook at RT for 10 min, then native amino-LCAA-CPG (250 mg, loading 75 umol/g) was added, the suspension was shook at RT for 20 h, then filtered, washed with DMF (20 mL×5), ACN (20 mL×5), DCM (20 mL×5) until TLC showed no spot at 254 nm in eluent. The solid support was dried under vacuum for 2 h to give solid support (260 mg). The unreacted amines on the solid support were capped by stirring with Ac2O/pyridine/N-methyl imidazole (90 μL/1.0 mL/80 μL) at RT for 1 h, washed with DMF (20 mL×5), CAN (20 mL×5), DCM (20 mL×5) until TLC showed no spot at 254 nm in eluent. The solid support was dried under vacuum for 15 h to give solid support 2-10 (260 mg). For loading calculation, take 6.5 mg of the dried loaded CPG and add 25 mL of 3% DCA in DCM. Shake the solution and measure UV absorbance at 500 nm. Make sure the absorbance value is below 1.0 unit to ensure no signal saturation. Then apply the following equation: Loading in umol/g=(total volume of DCA added in mL)*(Abs value at 500 nm)*1000)/(76*(mg of CPG taken))
Total volume of DCA added in mL=25 mL
Abs value at 500 nm=0.7721
mg of CPG taken=6.5 mg
Loading in umol/g=((25)*(0.7721)*1000)/(76*(6.5))=39 umol/g.

### Example 3

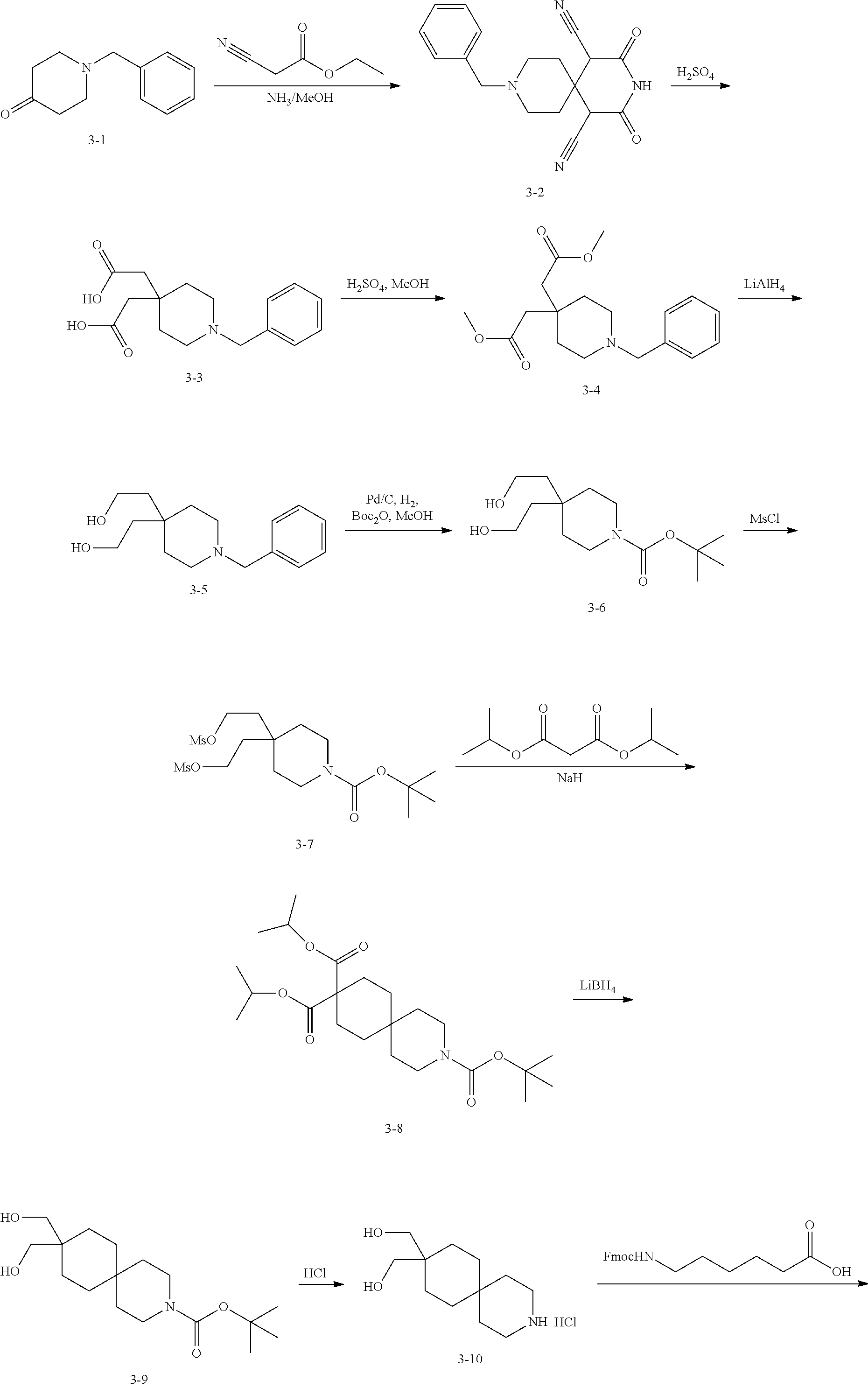

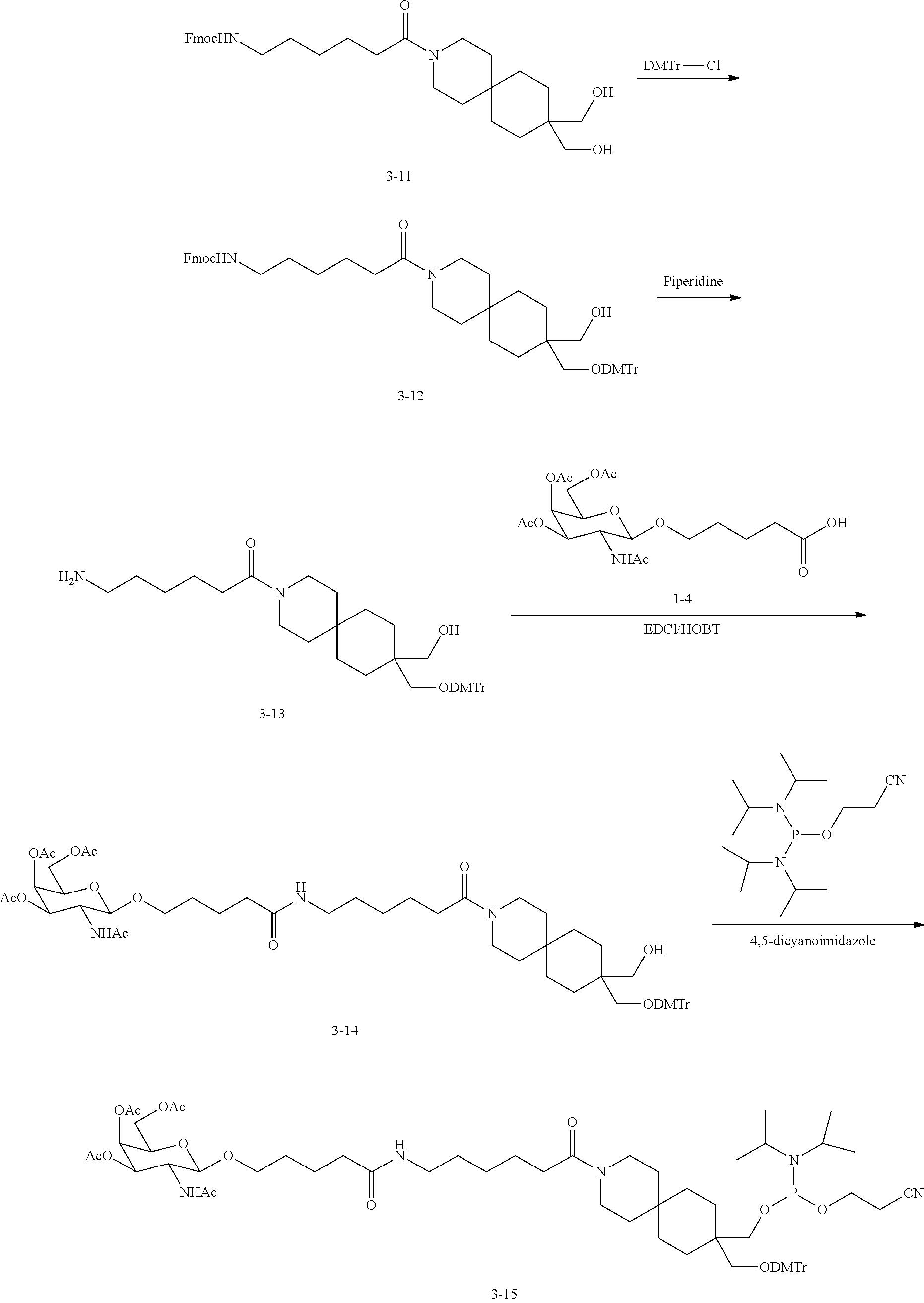

### Compound 3-2:

To a solution of Compound 3-1 (40 g, 211.36 mmol) in NH₃/MeOH (7 M, 192 mL) was added ethyl 2-cyanoacetate (47.82 g, 422.71 mmol) dropwise, and the mixture was stirred overnight. LC-MS showed full conversion, and the reaction was filtered. The filter cake was triturated in PE (100 mL), and filtered. The filter cake was dried to afford compound 3-2 as a solid (41 g, 44% yield).

Mass calc. for C₁₈H₁₈N₄O₂: 322.1; found: 323.1 [M+H]⁺, ESI.

### Compound 3-3:

Compound 3-2 (80 g, 248.17 mmol) was dissolved in sulfuric acid solution (conc. H₂SO₄/water=1:1, v/v, 160 mL), and stirred at 120° C. for 5 h. LC-MS showed full conversion, and the reaction mixture was adjusted to pH 9-10 by addition of NaOH (30% aq.), and re-adjusted to pH 4-5 by adding 1 M HCl solution. The reaction was then filtered and the filtrate was concentrated to dryness to afford crude compound 3-3 (72 g, 95% yield). The crude product was used directly without further purification.

Mass calc. For C₁₆H₂₁NO₄: 291.2; found: 292.3 [M+H]⁺, ESI.

### Compound 3-4:

Compound 3-3 (72.3 g crude) in MeOH (1600 mL) and H₂SO₄ (160 mL, conc. H₂SO₄/water=1:1, v/v) was stirred at 85° C. for 4 h. LC-MS showed full conversion. The reaction mixture was filtered and concentrated to dryness, and purified by silica gel column (0-25% EA in PE) to afford compound 3-4 (62.5 g, 78% yield over 2 steps).

Mass calc. for C₁₈H₂₅NO₄: 319.2; found: 320.3 [M+H]⁺, ESI.

### Compound 3-5:

To a solution of compound 3-4 (40 g, 125.2 mmol) in THF (295 mL) was added LiAlH₄ (14.3 g, 375.1 mmol) at 0° C. portion-wise and stirred for 1 h. LC-MS showed full conversion, and ethyl acetate (100 mL) was added to the reaction mixture, and adjusted to pH 9-10 by adding 10% NaOH (aq). The mixture was then filtered and washed by ethyl acetate (50 mL×2). The combined filtrate was concentrated to dryness to afford crude compound 3-5 (23 g, 83% yield). The crude product was used in the next step without further purification.

Mass calc. C₁₆H₂₅NO₂ for 263.2; found: 264.3 [M+H]⁺, ESI.

### Compound 3-6:

Compound 3-5 (20 g, 75.94 mmol), Boc2O (16.57 g, 75.94 mmol) and Pd/C (10% Pd on carbon, wet, ˜55% water) in MeOH (150 mL) was stirred at room temperature under 1 atm H₂ overnight. LC-MS showed full conversion, and the reaction mixture was filtered and concentrated to afford Compound 3-6 as an oil (20 g, 96% yield), which was used without further purification.

Mass calc. C₁₄H₂₇NO₄ for 273.2; found: 274.1 [M+H]⁺, ESI.

### Compound 3-7:

To a solution of compound 3-6 (58 g, 212.17 mmol) in triethylamine (85.88 g, 848.67 mmol, 118.37 mL) and DCM (1.16 L) was added MsCl (72.91 g, 636.51 mmol). The reaction was stirred at r.t. overnight. LC-MS showed full conversion (by EtOH quenching, and detection of diether). The reaction mixture was diluted by DCM (IL) and washed by 10% citric acid and then NaHCO₃ (sat. aq.), and dried to afford compound 3-7 as an oil (87 g, 95% yield), which was used directly for the next step without further purification.

Mass calc. for C₁₆H₃₁NO₈S₂: 429.2; found: 430.3 [M+H]⁺, ESI.

### Compound 3-8:

NaH (60% in mineral oil, 3.72 g, 93.12 mmol) was suspended in anhydrous DMF (60 mL) under nitrogen, diisopropyl malonate (8.76 g, 46.56 mmol) was added. The reaction mixture was stirred at r.t for 30 min, and a clear solution was formed. KI (1.55 g, 9.31 mmol) was added, followed by compound 3-7 (20 g, 46.56 mmol). The reaction mixture was stirred at 70° C. for 30 min and 140° C. for another 30 min. Full conversion showed by LC-MS. The reaction was cooled to r.t., and diluted by ethyl acetate (300 mL) and washed by citric acid (10% aq., 200 mL×2) and NaHCO₃ (sat. aq., 200 mL). The organic phase was concentrated and purified by silica gel column (0-10% EA in PE) to afford compound 3-8 as a colorless oil (3.55 g, 18% yield).

Mass calc. for C₂₃H₃₉NO₆: 425.3; found: 426.2 [M+H]⁺, ESI.

¹H NMR (400 MHz, DMSO-d₆) δ 4.96-4.90 (m, 2H), 3.27 (br, 4H), 1.86 (t, J=10.8 Hz, 4H), 1.38 (br, 13H), 1.28 (s, 4H), 1.17-1.16 (m, 12H).

### Compound 3-9:

To compound 3-8 (7.5 g, 17.62 mmol) was added LiBH₄ (172 mL, 1 M, in THF) at 0° C., and the mixture was stirred overnight. LC-MS showed full conversion. To the reaction mixture was added ethyl acetate (10 mL), then NaOH (10% aq., 20 mL) and filtered. The filtrate was diluted by water (200 mL) and extracted by ethyl acetate (50 mL×3), and concentrated to afford compound 3-9 (6.35 g, 98% yield) as a colorless oil, which was used directly for the next step without further purification.

Mass calc. for C₁₇H₃₁NO₄: 313.2; found: 314.2 [M+H]⁺, ESI.

### Compound 3-10:

Compound 3-9 (6.35 g, 20.26 mmol) was mixed with HCl (63.5 mL, 4M in dioxane) and the mixture was stirred at r.t. for 3 h. LC-MS showed full conversion. The reaction mixture was concentrated to dryness and azeotropically dried with toluene to afforded compound 3-10 (7 g, 99% yield), which was used directly for the next step without further purification.

Mass calc for C₁₂H₂₃NO₂ (free base): 213.2; found: 214.2 [M+H]⁺, ESI.

¹H NMR (400 MHZ, D₂O) δ 3.39 (br, 4H), 3.18 (t, J=11.6 Hz, 4H), 1.59 (t, J=12.0 Hz, 4H), 1.36-1.33 (m, 4H), 1.25 (t, J=12.4 Hz, 4H).

### Compound 3-11:

A solution of compound 3-10 (5 g, 20.02 mmol), DIEA (6.47 g, 50.04 mmol, 8.72 mL), HOBT (3.25 g, 24.02 mmol), EDCI (4.60 g, 24.02 mmol) in DCM (150 mL) was stirred at 0° C. for 30 min, then Fmoc-6-aminohexanoic acid (5.66 g, 16.01 mmol) was added, and the mixture was stirred at r.t. for 1 h. LC-MS showed full conversion. The reaction mixture was diluted by additional DCM (100 mL), washed by citric acid (10% aq., 200 mL), NaHCO₃ (sat. aq., 200 mL), and water (200 mL), dried over Na₂SO₄, and concentrated to afford crude product, which was purified by silica gel column (0-10% EA in PE) to afford compound 3-11 as a gum (3.65 g, LC-MS purity 92%, 33% yield).

Mass calc. for C₃₃H₄₄N₂O₅: 548.3; found: 549.3 [M+H]⁺, ESI.

¹H NMR (400 MHZ, DMSO-d₆) δ 7.88 (d, J=7.5 Hz, 2H), 7.84 (d, J=7.5 Hz, 2H), 7.41 (t, J=7.3 Hz, 2H), 7.34 (t, J=7.3 Hz, 2H), 6.65 (t, J=5.3 Hz, 1H), 6.28 (s, 2H), 4.36-4.18 (m, 3H), 3.36 (dt, J=19.9, 5.2 Hz, 4H), 3.24 (s, 4H), 2.92-2.87 (m, 2H), 2.24 (t, J=7.4 Hz, 2H), 1.49-1.26 (m, 18H).

### Compound 3-12:

A solution of compound 3-11 (2.55 g, 4.65 mmol), 4,4′-(chloro(phenyl)methylene) bis(methoxybenzene) (1.57 g, 4.65 mmol), DIEA (600.6 mg, 4.65 mmol), DMAP (56.8 mg, 0.47 mmol) in DCM (51 mL) was stirred at r.t. overnight. LC-MS showed partial conversion. Then reaction mixture was concentrated and purified by silica gel column (10-40% EA in PE to elude impurities and then 5-10% MeOH with 0.1% NH₃·H₂O in DCM) to afford compound 3-12 (2.75 g, 69% yield).

¹H NMR (400 MHZ, DMSO-d₆) δ 7.88-7.87 (m, 2H), 7.69-7.68 (m, 2H), 7.41-7.37 (m, 4H), 7.33-7.25 (m, 9H), 6.89-6.80 (m, 5H), 4.41 (t, J=5.0 Hz, 1H), 4.28 (d, J=6.8 Hz, 2H), 4.20 (t, J=6.8 Hz, 1H), 3.72 (s, 8H), 3.40-3.39 (m, 2H), 3.32-3.27 (m, 4H), 2.96 (q, J=6.3 Hz, 2H), 2.22 (t, J=7.9 Hz, 2H), 1.48-0.86 (m, 18H).

### Compound 3-13:

To a solution of compound 3-12 (1.7 g, 2.00 mmol) in MeOH (51 mL) was added piperidine (5.1 mL) and the mixture was stirred at r.t. for 3 h. LC-MS showed full conversion. The reaction mixture was concentrated, and azeotropically dried with xylene. The residue was purified by silica gel column (0-10% MeOH with 0.1% NH₃·H₂O in DCM) to afford compound 3-13 as a gum (1.1 g, 87% yield).

Mass calc. for C₃₉H₅₂N₂O₅: 628.4; found: 629.4 [M+H]⁺, ESI.

### Compound 3-14:

A solution of compound 3-13 (0.94 g, 1.49 mmol), DIEA (482.98 mg, 3.74 mmol), HOBT (242.38 mg, 1.79 mmol), EDCI (343.87 mg, 1.79 mmol) in DCM (29 mL) was stirred at 0° C., then compound 1-4 (936.36 mg, 2.09 mmol) was added, and the mixture was stirred at r.t. for 1 h. LC-MS showed full conversion. The reaction mixture was diluted by additional DCM (20 mL), and washed by NaHCO₃ (sat. Aq., 50 mL), and water (50 mL), concentrated and purified by prep-HPLC (C18 column, ACN/water) to afford compound 3-14 (700 mg, 44% yield).

Mass calc for C₅₈H₇₉N₃O₁₅: 1057.6; found: 1080.5 [M+Na]⁺, ESI.

¹H NMR (400 MHZ, DMSO-d₆) δ 7.82 (d, J=9.2 Hz, 1H), 7.69 (t, J=5.5 Hz, 1H), 7.40-7.38 (m, 2H), 7.32-7.19 (m, 7H), 6.89-6.87 (m, 4H), 5.21 (d, J=3.3 Hz, 1H), 4.97 (dd, J=11.2, 3.4 Hz, 1H), 4.48 (d, J=8.5 Hz, 1H), 4.40 (t, J=5.0 Hz, 1H), 4.02 (s, 3H), 3.91-3.83 (m, 1H), 3.73-3.68 (m, 7H), 3.43-3.39 (m, 3H), 3.32-3.27 (m, 2H), 2.99 (q, J=6.6 Hz, 2H), 2.87 (s, 2H), 2.22 (t, J=7.3 Hz, 2H), 2.10 (s, 3H), 2.03 (t, J=7.0 Hz, 2H), 1.99 (s, 3H), 1.89 (s, 3H), 1.77 (s, 3H), 1.53-1.03 (m, 20H), 0.90-0.83 (m, 2H).

### Compound 3-15:

To a solution of compound 3-14 (670.0 mg, 0.61 mmol) in anhydrous DCM (3.0 mL) was added 4,5-dicyanoimidazole (96.0 mg, 0.81 mmol) and 2-Cyanoethyl N,N,N′,N′-tetraisopropylphosphorodiamidite (324 mg, 1.08 mmol) at rt and stirred for 1 h. LCMS showed the starting material was consumed completely.

The solution was quenched by water (100 mL), washed with brine (300 mL×3) and dried over by Na₂SO₄. Then the solution was concentrated under reduced pressure and the residue was purified by Flash-Prep-HPLC with the following conditions (Column: C18 silica gel; mobile phase: CH₃CN/H₂O=1/1 increasing to CH₃CN/H₂O=1/0 within 20 min; Detector: UV 254 nm). This resulted in giving compound 3-15 as a white solid (600 mg, 83% yield).

Mass calc. for C₆₇H₉₆N₅O₁₆P: 1257.66; found: 1204.5 [M-CH₂CH₂CN], ESI.

¹HNMR (600 MHz, DMSO-d₆) δ 7.81-7.67 (m, 2H), 7.40-7.19 (m, 9H), 6.88-6.86 (m, 4H), 5.21 (d, J=6.0 Hz, 1H), 4.98-4.94 (m, 1H), 4.49 (d, J=6.0 Hz, 1H), 4.04-3.83 (m, 4H), 3.73-3.32 (m, 15H), 3.01-2.89 (m, 4H), 2.73-2.70 (m, 2H), 2.24-2.20 (m, 2H), 2.11-1.76 (m, 15H), 1.49-0.91 (m, 34H).

³¹PNMR (242 MHZ, DMSO-d₆) δ 146.21, 146.19.

### Example 4

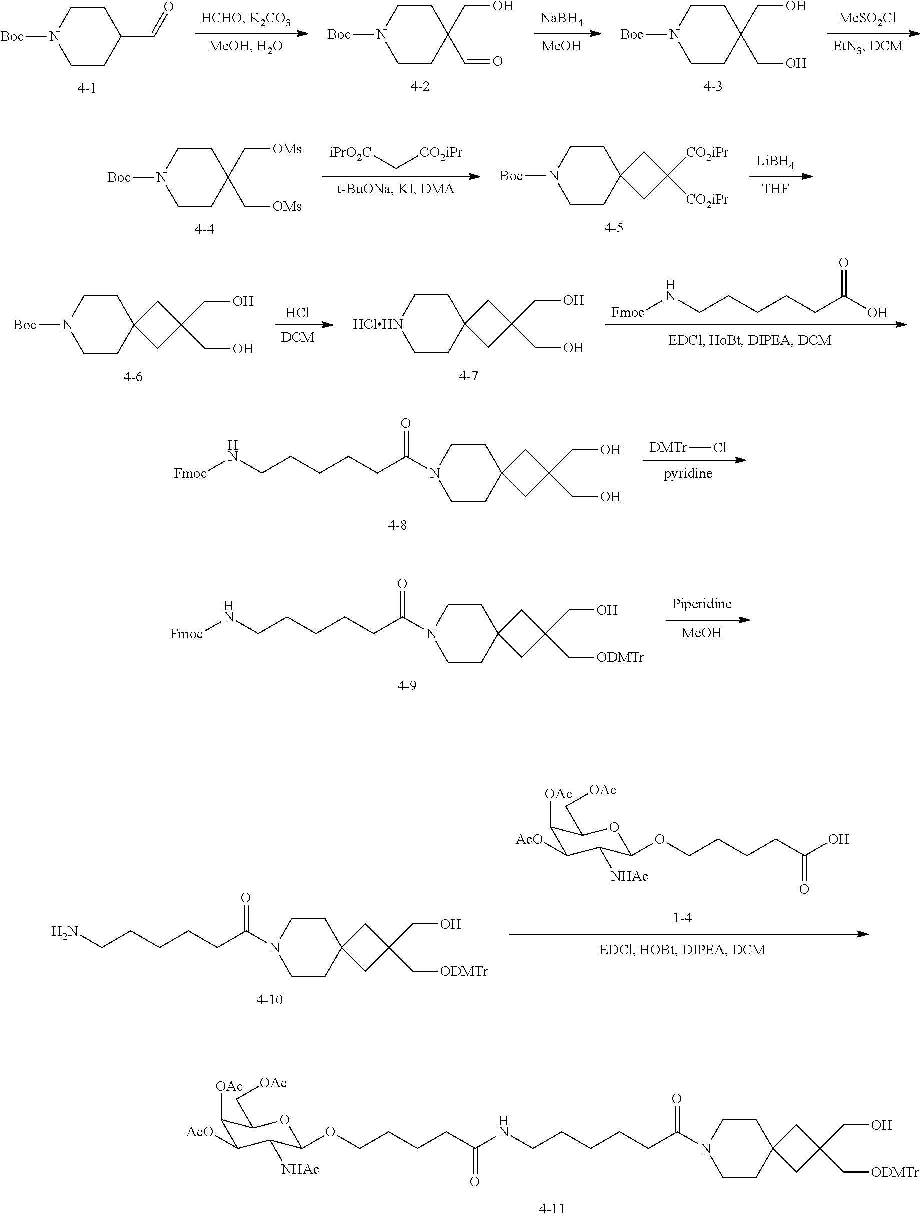

### Compound 4-2:

Potassium carbonate (8.10 g, 58.61 mmol) was added to a solution of MeOH (80 mL), H₂O (60 mL), formaldehyde (3.52 g, 117.22 mmol) and compound 4-1 (25 g, 117.22 mmol) at 0° C. The reaction mixture was stirred at 0° C. overnight. The reaction mixture was concentrated to 70% of its volume, and the residue was extracted three times with ethyl acetate. The organic phase was washed with water, brine, dried over anhydrous sodium sulfate, and then concentrated to give crude product 4-2 (27 g, 95% yield), which was used for the next step without further purification.

Mass calc. for C₁₂H₂₁NO₄: 243.2; found: 244.1 [M+H]⁺, ESI.

### Compound 4-3:

To a solution of compound 4-2 (35 g, 143.86 mmol) in methanol (28 ml) was added sodium borohydride (11.97 g, 316.48 mmol) at 0° C. in about five minutes. The reaction mixture was stirred at 0° C., then warmed to room temperature and stirred for 5 h. The reaction was quenched with saturated ammonium chloride solution, then concentrated, extracted with ethyl acetate (EA) for three times and washed with brine, then recrystallized with EA:PE=1:3 to give compound 4-3 (19.6 g, 54% yield).

Mass calc. C₁₂H₂₃NO₄ for: 245.2; found: 146.1 [M+H-Boc]⁺, ESI.

### Compound 4-4:

Compound 4-3 (30 g, 122.29 mmol), triethylamine (30.94 g, 305.73 mmol, 42.64 mL) and DCM (300 mL) were mixed, then the mixture was cooled to −30° C., then MsCl (35.02 g, 305.73 mmol) was added slowly. After stirring at room temperature for 1.5 h, the reaction mixture was extracted with 400 mL of water, washed with 100 mL of 10% citric acid aqueous solution, dried over anhydrous sodium sulfate. After concentration, the residue was purified by silica gel column to afford compound 4-4 as a light-yellow oily liquid (40 g, 81% yield).

Mass calc. for C₁₄H₂₇NO₈S₂: 401.1; found: 302.1 [M+H-Boc]⁺, ESI.

### Compound 4-5:

To a mixture of t-BuONa (9.57 g, 99.63 mmol) in DMA (50 mL) under nitrogen was added slowly diisopropyl malonate (14.06 g, 74.72 mmol). After stirring for 20 mins, compound 4-4 (20 g, 49.81 mmol), KI (4.13 g, 24.91 mmol) were added. The reaction mixture was warmed to 140° C. and stirred overnight. The reaction was quenched by adding saturated ammonium chloride and extracted with ethyl acetate. After concentration, the residue was purified by silica gel column (PE:EA) to afford compound 4-5 (6 g, 30% yield).

Mass calc. for C₂₁H₃₅NO₆: 397.3; found: 398.2 [M+H]⁺, ESI.

¹H NMR (400 MHZ, DMSO) δ 4.98-4.90 (m, 2H), 3.22-3.20 (m, 4H), 2.25 (s, 4H), 1.42 (t, J=5.6 Hz, 4H), 1.38 (s, 9H), 1.17 (d, J=6.0 Hz, 12H).

### Compound 4-6:

To a solution of compound 4-5 (9.75 g, 24.53 mmol) in anhydrous THF (200 mL) was added lithium borohydride (10.69 g, 490.56 mmol) at 0° C. The reaction mixture was warmed slowly to room temperature and stirred overnight. Then ethyl acetate (200 mL) was added to the reaction mixture, and it was extracted by water (150 mL×3), then washed with saturated sodium chloride, dried over anhydrous sodium sulfate, concentrated to obtain crude product 4-6 (6.52 g, 93% yield), which was used for the next step without further purification.

Mass calc. for C₁₅H₂₇NO₄: 285.2; found: 186.2 [M+H-Boc]⁺, ESI.

### Compound 4-7:

To a solution of product 4-6 (7.52 g, 26.35 mmol) in DCM (100 mL) at 0° C. was added HCl (4M in 1,4-dioxane, 21.52 mL) in 15 min. The reaction mixture was warmed to room temperature and stirred for 3 h. The reaction mixture was filtered, and the filter cake was washed with dichloromethane (×3) to give the crude product 4-7 (3.7 g, 76% yield).

Mass calc. for C₁₀H₁₉NO₂ (free base): 185.1 m/z; found: 186.2 [M+H]⁺, ESI.

### Compound 4-8:

A mixture of Fmoc-6-aminohexanoic acid (1.91 g, 5.41 mmol), 1-hydroxybenzotriazole (HOBT, 609.41 mg, 4.51 mmol), EDCI (864.59 mg, 4.51 mmol), DIEA (582.89 mg, 4.51 mmol, 785.56 μL) in DCM (10 mL) was stirred at room temperature for 30 min, then it was added to the solution of compound 4-7 (1 g, 4.51 mmol) in DCM and the mixture was stirred for 4 hours. The reaction mixture was washed with saturated sodium bicarbonate solution, brine and dried over anhydrous sodium sulfate. After concentration, the residue was purified by silica gel column to afford compound 4-8 (1.35 g, 58% yield).

Mass calc. for C₃₁H₄₀N₂O₅: 520.3; found: 521.2 [M+H]⁺, ESI.

¹H NMR (400 MHZ, DMSO) δ 7.88 (d, J=7.5 Hz, 2H), 7.68 (d, J=7.4 Hz, 2H), 7.39 (d, J=7.5 Hz, 4H), 7.34-7.18 (m, 10H), 6.89 (d, J=8.8 Hz, 4H), 4.64 (t, J=4.7 Hz, 1H), 4.28 (d, J=6.8 Hz, 2H), 4.20 (t, J=6.8 Hz, 1H), 3.73 (s, 6H), 3.44 (d, J=5.0 Hz, 2H), 3.30-3.10 (m, 4H), 3.01 (s, 2H), 3.00-2.90 (m, 2H), 2.21 (t, J=7.2 Hz, 2H), 1.56 (d, J=11.9 Hz, 2H), 1.46-1.30 (m, 8H), 1.21-1.00 (m, 4H).

### Compound 4-9:

The mixture of DMTr-Cl (488.07 mg, 1.44 mmol) and compound 4-8 (0.75 g, 1.44 mmol) in pyridine (6 ml) was stirred at room temperature overnight. The reaction mixture was concentrated under vacuum. The residue was re-dissolved in ethyl acetate with 1% pyridine and dried together with alumina for column chromatography purification (0-10% MeOH, 0.1% TEA in DCM) on alumina to afford compound 4-9 (0.39 g, 32.9% yield).

¹H NMR (400 MHZ, DMSO) δ 7.39 (d, J=7.3 Hz, 2H), 7.34-7.18 (m, 7H), 6.89 (d, J=8.8 Hz, 4H), 4.63 (s, 1H), 3.73 (s, 6H), 3.43 (s, 2H), 3.30-3.18 (m, 6H), 3.05 (s, 2H), 2.22 (t, J=7.4 Hz, 2H), 1.57 (d, J=12.1 Hz, 2H), 1.47-1.08 (m, 12H).

### Compound 4-10:

Compound 4-9 (1.4 g, 1.70 mmol) was dissolved in MeOH (40 mL), then piperidine (4 ml) was added. The mixture was stirred at room temperature overnight. The reaction mixture was concentrated under vacuum. The piperidine residue was further removed by azeotropic drying with xylene. Then the residue was purified by silica gel column (DCM:MeOH=10:1, methanol contains 10% ammonia) to afford compound 4-10 (1 g, 97% yield).

Mass calc. for C₃₇H₄₈N₂O₅: 600.4; found: 601.4 [M+H]⁺, ESI.

¹H NMR (400 MHZ, DMSO-d₆) δ 7.40-7.39 (m, 2H), 7.33-7.21 (m, 7H), 6.89 (d, J=8.8 Hz, 4H), 4.63 (s, 1H), 3.73 (s, 6H), 3.44-3.16 (m, 8H), 3.01-3.00 (m, 2H), 2.24-2.21 (m, 2H), 1.57 (d, J=12.2 Hz, 2H), 1.45-1.33 (m, 7H), 1.27-1.21 (m, 4H), 1.09 (s, 1H).

### Compound 4-11:

The mixture of compound 1-4 (959.23 mg, 2.14 mmol), HOBt (289.68 mg, 2.14 mmol), EDCI (410.98 mg, 2.14 mmol), DIEA (395.82 mg, 3.06 mmol, 533.45 μL) in DCM (10 mL) was stirred for 20 min, then it was added to the solution of compound 4-10 (920 mg, 1.53 mmol) in DCM (5 mL) and stirred at room temperature overnight. Water was added to quench the reaction and the organic layer was separated and concentrated. The residue was purified by prep-HPLC to give compound 4-11 (1 g, 63% yield).

Mass calc. for C₅₆H₇₅N₃O₁₅: 1029.5; found: 1052.3 [M+Na]⁺, ESI.

¹H NMR (400 MHZ, DMSO-d₆) δ 7.82 (d, J=9.2 Hz, 1H), 7.79 (t, J=5.0 Hz, 1H), 7.40-7.19 (m, 9H), 6.90-6.88 (m, 2H), 5.21 (d, J=3.2 Hz, 1H), 4.97 (dd, J=11.2, 3.3 Hz, 1H), 4.62 (t, J=4.9 Hz, 1H), 4.48 (d, J=8.4 Hz, 1H), 4.02 (s, 3H), 3.87 (dd, J=20.4, 9.6 Hz, 1H), 3.97-3.67 (m, 7H), 3.45-3.38 (m, 3H), 3.30-3.13 (m, 4H), 3.05-2.96 (m, 4H), 2.22 (t, J=7.3 Hz, 2H), 2.10 (s, 3H), 2.04-1.99 (m, 5H), 1.89 (s, 3H), 1.77 (s, 3H), 1.59-1.21 (m, 18H).

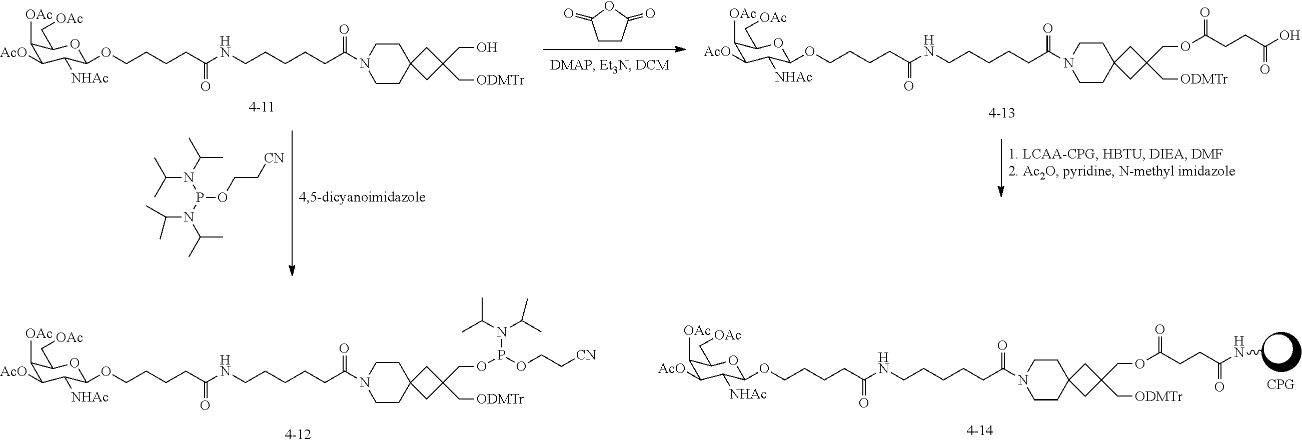

### Compound 4-12:

To a solution of compound 4-11 (750.0 mg, 0.75 mmol) in anhydrous DCM (7.0 mL) was added 4,5-dicyanoimidazole (80.0 mg, 0.67 mmol) and 2-Cyanoethyl N,N,N′,N′-tetraisopropylphosphorodiamidite (270 mg, 0.90 mmol) at rt and stirred for 1 h, LCMS showed the starting material was consumed completely.

The solution was quenched by water (100 mL), washed with brine (300 mL×3) and dried over by Na₂SO₄. Then the solution was concentrated under reduced pressure and the residue was purified by Flash-Prep-HPLC with the following conditions (Column, C18 silica gel; mobile phase, CH₃CN/H₂O=1/1 increasing to CH₃CN/H₂O=1/0 within 20 min; Detector, UV 254 nm). This resulted in to give 4-12 as a white solid (500 mg, 53% yield).

Mass calc. for C₆₅H₉₂N₅O₁₆P: 1229.63; found: 1129.0 [M-N(CH(CH₃)₂)₂]⁺, ESI.

¹HNMR (600 MHZ, DMSO-d₆): δ 7.44-7.18 (m, 9H), 6.85-6.83 (m, 4H), 6.52-6.44 (m, 2H), 5.26 (d, J=6.0 Hz, 1H), 4.99-4.96 (m, 1H), 4.49 (d, J=6.0 Hz, 1H), 4.13-3.89 (m, 4H), 3.79-3.55 (m, 13H), 3.44-3.48 (m, 1H), 3.35-3.07 (m, 8H), 2.58-2.56 (m, 2H), 2.24-1.89 (m, 16H), 1.62-1.11 (m, 31H).

³¹PNMR (242 MHZ, DMSO-d₆) δ 147.17, 147.16.

### Compound 4-13:

To a solution of compound 4-11 (80 mg, 0.080 mmol) in anhydrous DCM (1.0 mL), DMAP (5 mg, 0.04 mmol) and TEA (24 mg, 0.24 mmol) were added, followed by adding succinic anhydride (20 mg, 0.2 mmol), the reaction mixture was stirred at rt for 3 h, LCMS showed the starting material was consumed completely, then the reaction mixture was diluted with DCM and washed with water (3 mL×4), then washed with brine (3 mL×4), the organic layer was concentrated to give compound 4-13 as a white solid (85 mg, 97% yield).

Mass calc. for C₆₀H₇₉N₃O₁₈: 1129.54; found: 1128.6 [M−H], ESI.

¹HNMR (600 MHZ, DMSO-d₆) δ 12.22 (s, 1H), 7.84-7.81 (m, 1H), 7.71-7.69 (m, 1H), 7.37-7.16 (m, 9H), 6.92-6.89 (m, 4H), 5.21 (d, J=6.0 Hz, 1H), 4.97-4.95 (m, 1H), 4.48 (d, J=12.0 Hz, 1H), 4.12-4.01 (m, 3H), 3.89-3.84 (m, 1H), 3.73-3.65 (m, 7H), 3.41-3.38 (m, 2H), 3.28-3.15 (m, 4H), 3.15-2.98 (m, 4H), 2.46-2.44 (m, 4H), 2.22-2.20 (m, 2H), 2.09-1.76 (m, 14H), 1.65-1.60 (m, 2H), 1.48-1.06 (m, 19H).

### Compound 4-14:

To a solution of compound 4-13 (85 mg, 0.077 mmol) and HBTU (53 mg, 0.14 mmol) in anhydrous DMF (1.5 mL), DIPEA (30 mg, 0.23 mmol) was added. The reaction mixture was shaken at rt for 10 min, then native amino-lcaa-CPG (250 mg, loading 75 umol/g) was added, the suspension was shaken at r.t. for 20 h, then filtered, washed with DMF (20 mL×5), ACN (20 mL×5), DCM (20 mL×5) until TLC showed no spot at 254 nm in eluent. The solid support was dried under vacuum for 2 h to give solid support (260 mg). The unreacted amines on the support were capped by stirring with Ac₂O/pyridine/N-methyl imidazole (90 μL/1.0 mL/80 μL) at rt for 1 h, washed with DMF (20 mL×5), ACN (20 mL×5) and DCM (20 mL×5) until TLC showed no spot at 254 nm in eluent. The solid support was dried under vacuum for 15 h to give solid support 4-14 (260 mg). For loading calculation, take 5.97 mg of the dried loaded CPG and add 25 mL of 3% DCA on DCM. Shake the solution and measure UV absorbance at 500 nm. Make sure the absorbance value is below 1.0 units to ensure no signal saturation. Then apply the following equation:

Loading in umol/g=(total volume of DCA added in mL)*(Abs value at 500 nm)*1000)/76*(mg of CPG taken)
Total volume of DCA added in mL=25 mL
Abs value at 500 nm=0.6689
Mg of CPG taken=5.97 mg
Loading in umol/g=((25)*(0.6689)*1000)/76*(5.97)=36.85 umol/g.

### Example 5

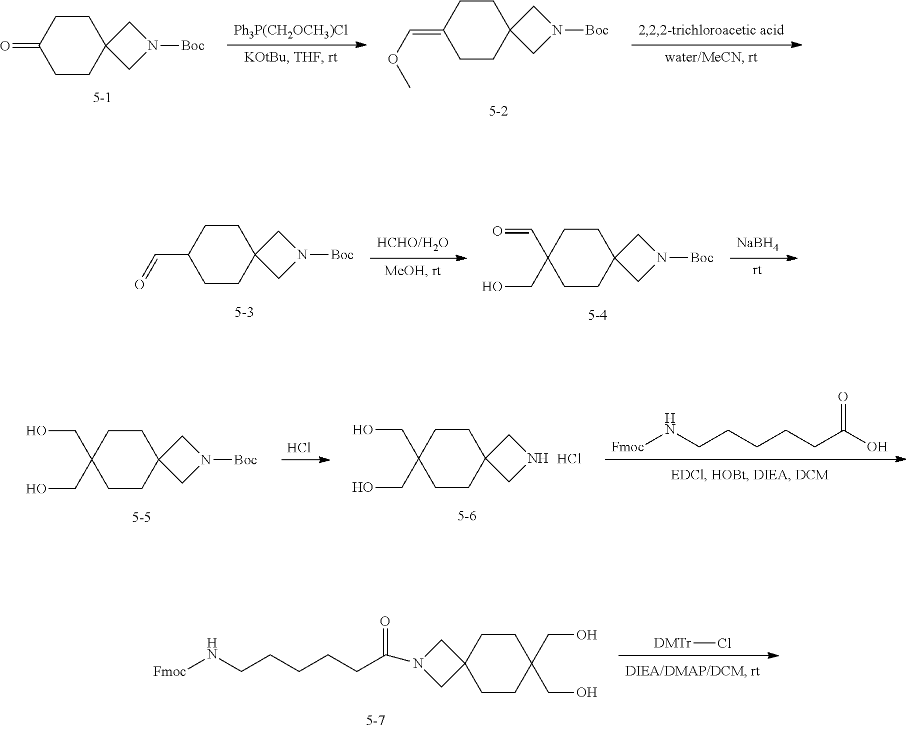

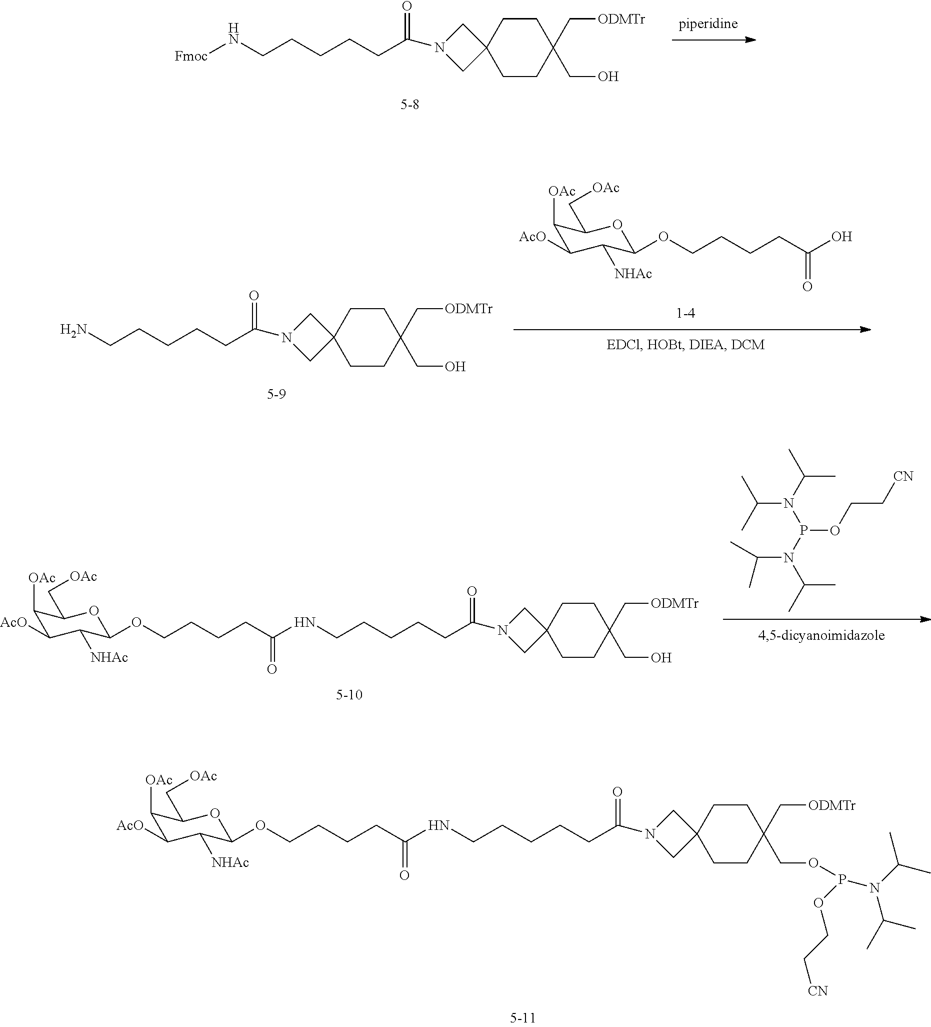

### Compound 5-2:

To a solution of methoxymethyl(triphenyl)phosphonium chloride (12.89 g, 37.61 mmol), KOtBu (4.69 g, 41.79 mmol) in THF (150 mL) under nitrogen at 0° C. was added compound 5-1 (10 g, 41.78 mmol). The reaction mixture was stirred at 25° C. overnight. TLC showed full conversion. The reaction was then adjusted to pH 6 by adding citric acid (10% aq.) and extracted by ethyl acetate (EA). The organic phase was concentrated and purified by flash column chromatography (0-10% EA in PE) to afford compound 5-2 as an oil (2.92 g, 37% yield).

Mass calc. for C₁₅H₂₅NO₃: 267.2; found: 268.2 [M+H]⁺, ESI.

### Compound 5-3:

To a mixture of compound 5-2 (8.2 g, 30.67 mmol) in MeCN (220 mL) was added 2,2,2-trichloroacetic acid (6.31 g, 38.64 mmol) in water (70 mL) and the mixture was stirred overnight. Full conversion by TLC. The reaction was then quenched by adjusting pH to 7-8 by NaHCO₃ (aq., sat.) and extracted by ethyl acetate. The organic phase was concentrated to afford compound 5-3 as an oil (7.8 g). The crude product was used directly for the next step without purification.

Mass calc. for C₁₄H₂₃NO₃: 253.2; found: 154.2 [M+H-Boc]⁺, ESI.

### Compound 5-4:

To a mixture of compound 5-3 (1.35 g, 5.33 mmol) and NaOH (21.32 mg, 532.89 μmol, 29.38 μL) in MeOH (30 mL) was added formaldehyde solution (454.47 mg, 15.13 mmol, 37% aq., 0.91 g). The reaction mixture was stirred at 25° C. for 36 h. The reaction mixture was concentrated to remove most of methanol, extracted by ethyl acetate. The organic phase was concentrated and purified by column (0-5% MeOH in DCM) to afford compound 5-4 as a solid (1.5 g, 96% yield over 2 steps).

Mass calc. for C₁₅H₂₅NO₄: 283.2; found: 184.2 [M+H-Boc]⁺, ESI.

### Compound 5-5:

To a solution of compound 5-4 (2.95 g, 10.41 mmol) in MeOH (90 mL) was added sodium borohydride (866.50 mg, 22.90 mmol). The reaction mixture was stirred for 1 h. LC-MS showed full conversion. The reaction mixture was adjusted to pH6 by citric acid (10% aq.), then it was concentrated to dryness and the residue was extracted by DCM and filtered. The filtrate was concentrated to afford compound 5-5 (2.95 g, 73% yield), which was used directly for the next step without purification.

Mass calc. for C₁₅H₂₇NO₄: 285.2; found: 271.2 [M+ACN+H−tBu]⁺, ESI.

### Compound 5-6:

To compound 5-5 (4 g, 14.02 mmol) was added HCl (4M in dioxane, 63.5 mL). The reaction mixture was stirred for 3 h. LC-MS showed full conversion. The reaction mixture was concentrated to afford compound 5-6 (3.5 g), which was used directly for the next step without purification.

Mass calc. for C₁₀H₁₉NO₂ (free base): 185.1; found: 186.1 [M+H]⁺, ESI.

### Compound 5-7:

A solution of Fmoc-6-aminohexanoic acid (3.19 g, 9.02 mmol), DIEA (2.91 g, 22.55 mmol, 3.93 mL), HOBT (1.46 g, 10.82 mmol), EDCI (2.08 g, 10.82 mmol) in DCM (12 mL) was stirred for 30 min at 0° C., then compound 5-6 (2 g, 9.02 mmol) was added. The reaction mixture was stirred at rt overnight. LC-MS showed full conversion. The reaction mixture was diluted by DCM (12 mL) and washed with citric acid (10% aq., 20 mL) and water. The organic phase was concentrated and purified by silica gel column (0-5% MeOH in DCM) to afford compound 5-7 (3.5 g, 34% yield).

Mass calc. for C₃₁H₄₀N₂O₅: 520.3; found: 521.2 [M+H]⁺, ESI.

¹H NMR (400 MHZ, DMSO-d₆) δ 7.91-7.68 (m, 4H), 7.44-7.28 (m, 5H), 4.32-4.28 (m, 4H), 4.20 (t, J=6.6 Hz, 1H), 3.69 (s, 2H), 3.43 (s, 2H), 3.22 (t, J=5.6 Hz, 4H), 2.02-1.98 (m, 3H), 1.55-1.52 (t, J=6.6 Hz, 4H), 2.26-2.23 (m, 4H), 1.24-1.16 (m, 8H).

### Compound 5-8:

To a solution of compound 5-7 (2.2 g, 4.23 mmol), DMAP (51.6 mg, 0.423 mmol) and DIEA (1.09 g, 8.46 mmol) in DCM (66 mL) was added DMTrCl (1.43 g, 4.23 mmol). The reaction mixture was stirred overnight at room temperature. LC-MS showed partial conversion. The reaction mixture was filtered through a pad of alumina and washed with DCM (50 mL). The filtrate was concentrated and purified by silica gel column (10-40% EA with 0.1% ammonia in PE, then 0-10% MeOH with 0.1% ammonia in DCM) to afford compound 5-8 (3.0 g, 86% yield).

Mass for calc. C₅₂H₅₈N₂O₇: 822.4; found: 845.3 [M+Na]⁺, ESI.

¹H NMR (400 MHZ, DMSO-d₆) δ 7.90-7.67 (m, 4H), 7.42-7.39 (m, 4H), 7.41-7.24 (m, 14H), 6.89-6.87 (m, 4H), 4.44 (t, J=7.6 Hz, 1H), 4.24-4.19 (m, 1H), 4.06-4.01 (m, 1H), 3.74-3.73 (m, 6H), 3.65-3.59 (m, 2H), 3.42-3.39 (m, 3H), 3.32 (br, 1H), 3.00-2.83 (m, 4H), 1.99-1.96 (m, 4H), 1.45-1.16 (m, 16H).

### Compound 5-9:

To a solution of compound 5-8 (3 g, 3.65 mmol) in MeOH (90 mL) was added piperidine (9 mL). The reaction mixture was stirred at rt overnight. LC-MS showed full conversion. The reaction mixture was concentrated and then further azeotropically distilled with xylene. The residue was purified by silica gel column chromatographic (0-10% MeOH with 1% TEA in DCM) to afford compound 5-9 (1.1 g, 91% yield).

Mass calc. for C₃₇H₄₈N₂O₅: 600.4; found: 601.3 [M+H]⁺, ESI.

¹H NMR (400 MHZ, DMSO-d₆) δ 7.40-7.21 (m, 9H), 6.89 (d, J=7.2 Hz, 4H), 3.74 (s, 6H), 3.67-3.61 (m, 2H), 3.39-3.33 (m, 2H), 2.86-2.62 (m, 4H), 1.99 (t, J=7.4 Hz, 2H), 1.51-1.21 (m, 16H).

### Compound 5-10:

A solution of compound 1-4 (2.09 g, 4.66 mmol), DIEA (1.08 g, 8.32 mmol, 1.45 mL), HOBT (539.77 mg, 3.99 mmol) and EDCI (765.80 mg, 3.99 mmol) in DCM (60 mL) was stirred at 0° C. for 30 min, then compound 5-9 (2 g, 3.33 mmol) was added. The reaction mixture was stirred overnight. The reaction mixture was concentrated and the residue was purified by prep-HPLC (C18, water/ACN) to afford compound 5-10 (1.0 g, 15% yield).

Mass calc. for C₅₆H₇₅N₃O₁₅: 1029.5; found: 1052.3 [M+Na]⁺, ESI.

¹H NMR (400 MHZ, DMSO-d₆) δ 7.83 (d, J=9.2 Hz, 1H), 7.71 (t, J=5.2 Hz, 1H), 7.40-7.38 (m, 2H), 7.32-7.19 (m, 5H), 6.90-9.87 (m, 2H), 5.22 (d, J=3.4 Hz, 1H), 4.97 (dd, J=11.2, 3.4 Hz, 1H), 4.49 (d, J=8.5 Hz, 1H), 4.42 (t, J=5.0 Hz, 1H), 4.03 (s, 2H), 3.91-3.84 (m, 2H), 3.74 (s, 6H), 3.67-3.61 (m, 1H), 3.44-3.37 (m, 4H), 3.00 (dd, J=12.7, 6.6 Hz, 2H), 2.85 (d, J=9.6 Hz, 2H), 2.11 (s, 3H), 2.05-1.96 (m, 7H), 1.90 (s, 3H), 1.78 (s, 3H), 1.52-1.18 (m, 18H).

### Compound 5-11:

To a solution of compound 5-10 (690.0 mg, 0.66 mmol) in anhydrous DCM (3.0 mL) was added 4,5-dicyanoimidazole mg, (96.0 0.81 mmol) and 2-Cyanoethyl N,N,N′,N′-tetraisopropylphosphorodiamidite (324 mg, 1.08 mmol) at rt and stirred for 1 h, LCMS showed the starting material was consumed completely. The solution was quenched by water (100 mL), washed with brine (300 mL×3) and dried over by Na₂SO₄. Then the solution was concentrated under reduced pressure and the residue was purified by Flash-Prep-HPLC with the following conditions (Column, C18 silica gel; mobile phase, CH₃CN/H₂O=1/1 increasing to CH₃CN/H₂O=1/0 within 20 min; Detector, UV 254 nm). This resulted in to give 5-11 as a white solid (460 mg, 56% yield).

Mass calc. for C₆₅H₉₂N₅O₁₆P: 1229.63; found: 1230.3 [M+H]⁺, ESI.

¹HNMR (600 MHZ, DMSO-d₆) δ 7.81-7.67 (m, 2H), 7.40-7.19 (m, 9H), 6.88-6.86 (m, 4H), 5.21 (d, J=6.0 Hz, 1H), 4.98-4.94 (m, 1H), 4.49 (d, J=6.0 Hz, 1H), 4.04-3.83 (m, 4H), 3.73-3.32 (m, 18H), 3.01-2.83 (m, 4H), 2.72-2.69 (m, 2H), 2.10-1.76 (m, 16H), 1.52-1.04 (m, 30H).

³¹PNMR (242 MHZ, DMSO-d₆) δ 146.33, 146.26.

### Example 6

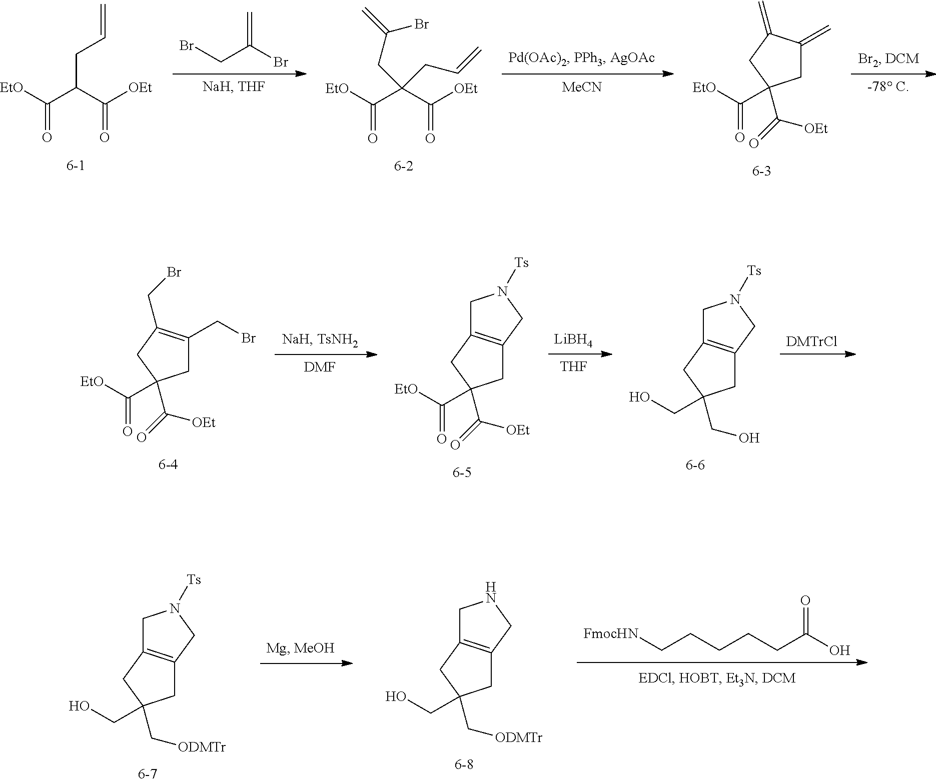

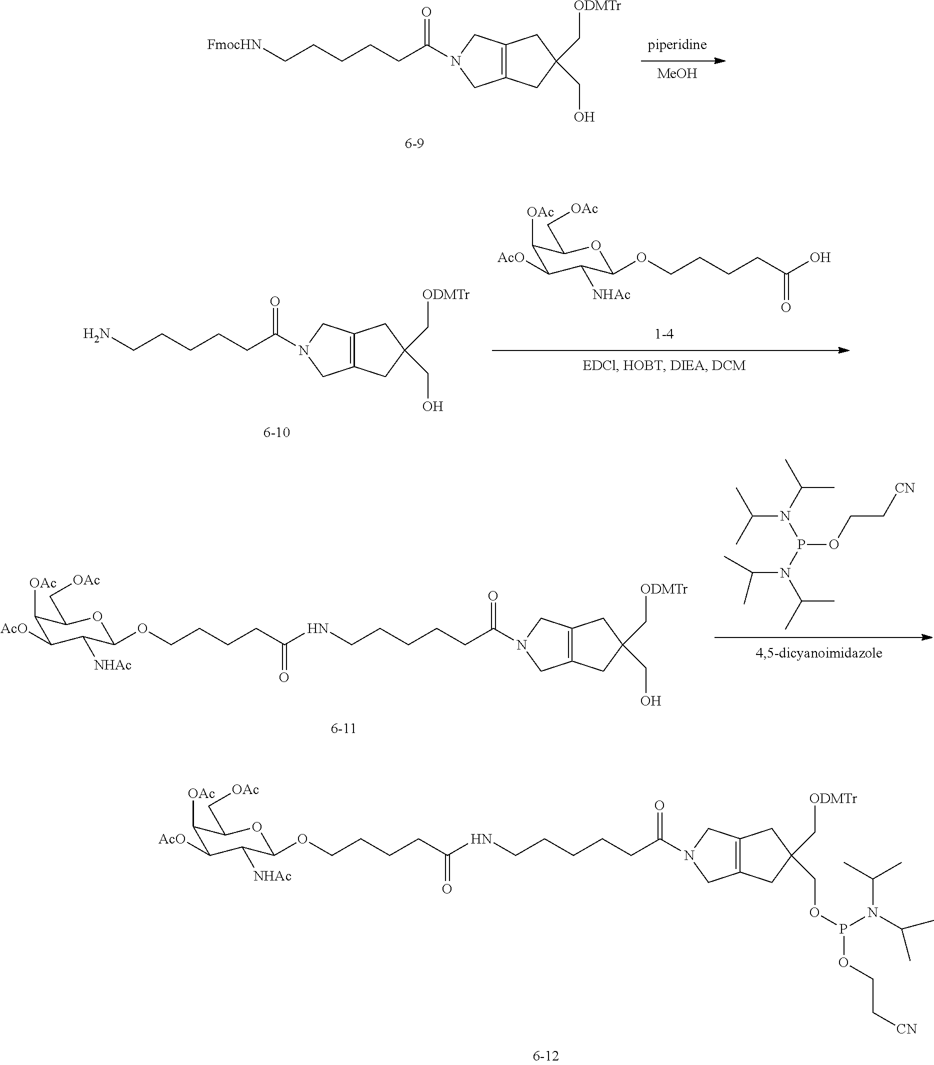

### Compound 6-2:

Compound 6-1 (20 g, 99.88 mmol) was dissolved in THF (200 mL). NaH (4.79 g, 119.86 mmol, 60% purity) was added to the reaction mixture and stirred 30 min at 0° C. 2,3-Dibromopropylene (20.96 g, 104.88 mmol) was slowly added to the reaction mixture. And the reaction was stirred at rt for 2 h. The reaction mixture was quenched with saturated NH₄Cl solution (100 mL), extracted with EA (200 mL×3), washed with brine (100 mL), dried over Na₂SO₄, concentrated under vacuum and purified by flash column chromatography on silica gel to give compound 6-2 as a colorless oil (31 g, 97% yield).

Mass calc. for C₁₃H₁₉BrO₄: 318.0; found: 319.0 [M+H]⁺, ESI.

¹H NMR (400 MHZ, CDCl₃) δ 5.71-5.58 (m, 3H), 5.14-5.10 (m, 2H), 4.25-4.13 (m, 4H), 3.14 (s, 2H), 2.7 (d, J=7.2 Hz, 2H), 1.26 (t, J=6.8 Hz, 6H).

### Compound 6-3:

Compound 6-2 (31 g, 97.12 mmol), Pd(OAc)₂ (2.18 g, 9.71 mmol), PPh₃ (5.09 g, 19.42 mmol) and AgOAc (19.45 g, 116.54 mmol) were dissolved in CAN (600 mL). The reaction mixture was stirred at 85° C. under N₂ atmosphere for 3 h. The reaction was cooled down to rt and filtered, concentrated under vacuum and purified by flash column chromatography on silica gel to give compound 6-3 as a colorless oil (21 g, 91% yield). Mass calc. for C₁₃H₁₈O₄: 238.1; found: 239.2 [M+H]⁺, ESI.

¹H NMR (400 MHZ, CDCl₃) δ 5.39 (t, J=2 Hz, 2H), 4.95 (t, J=1.6 Hz, 2H), 4.18 (q, J=7.2 Hz, 4H), 3.03 (t, J=7.2 Hz, 4H), 1.24 (t, J=7.2 Hz, 6H).

### Compound 6-4:

Compound 6-3 (21 g, 88.13 mmol) was dissolved in DCM (300 mL) and cooled down to −78° C. Br₂ (14.08 g, 88.13 mmol, 4.51 mL) was dissolved in DCM (100 mL) and added to the reaction mixture over 1 h. The mixture was stirred at −78° C. for 1 h. The reaction mixture was quenched with saturated Na₂SO₃ solution (100 mL), extracted with DCM (200 mL×3), washed with brine (100 mL), dried over Na₂SO₄, concentrated under vacuum and purified by flash column chromatography on silica gel to give compound 6-4 as a pale yellow oil (28 g, 80% yield).

Mass calc. for C₁₃H₁₈Br₂O₄: 396.0; found: 397.0 [M+H]⁺, ESI.

¹H NMR (400 MHZ, CDCl₃) δ: 4.21 (q, J=7.2 Hz, 4H), 4.00 (s, 4H), 3.19 (s, 4H), 1.26 (t, J=7.2 Hz, 6H).

### Compound 6-5:

4-methylbenzenesulfonamide (12.05 g, 70.35 mmol) was dissolved in DMF (260 mL). NaH (6.19 g, 154.77 mmol) was added to the reaction mixture at 0° C. And the reaction mixture was stirred at 0° C. for 30 min. Compound 6-4 (28 g, 70.35 mmol) in DMF (50 mL) was added to the reaction mixture slowly. The mixture was stirred at rt for 1 h. The reaction mixture was quenched with saturated NH₄Cl solution (500 mL), extracted with EA (200 mL×3), washed with water (500 mL×3), brine (300 mL), dried over Na₂SO₄, concentrated under vacuum and purified by flash column chromatography on silica gel to give compound 6-5 as a colorless solid (22 g, 76% yield).

Mass calc. for C₂₀H₂₅NO₆S: 407.1; found: 408.2 [M+H]⁺, ESI.

¹H NMR (400 MHZ, CDCl₃) δ 7.70-7.68 (m, 2H), 7.32-7.30 (m, 2H), 4.17 (q, J=7.2 Hz, 4H), 3.96 (s, 4H), 2.89 (s, 4H), 2.42 (s, 3H), 1.22 (t, J=7.2 Hz, 6H).

### Compound 6-6:

Compound 6-5 (22 g, 54.05 mmol) was dissolved in THF (300 mL). LiBH₄ (11.8 g, 540.5 mmol) was added to the reaction mixture at 0° C. The reaction was stirred at rt for 1 h. The reaction mixture was quenched with water (100 mL), extracted with EA (200 mL×3), washed with brine (100 mL), dried over Na₂SO₄, concentrated under vacuum and purified by flash column chromatography on silica gel to give compound 6-6 as a white solid (14.8 g, 89% yield).

Mass calc. for C₁₆H₂₁NO₄S: 323.1; found: 324.1 [M+H]⁺, ESI.

¹H NMR (400 MHZ, DMSO-d₆) δ 7.70-7.68 (m, 2H), 7.43-7.41 (m, 2H), 4.56 (t, J=5.2 Hz, 2H), 3.26 (d, J=4.8 Hz, 4H), 2.84 (s, 4H), 2.39 (s, 3H), 1.95 (s, 4H).

### Compound 6-7:

Compound 6-6 (4.1 g, 12.68 mmol) and DIEA (4.92 g, 3.83 mmol) were dissolved in DCM (20 mL). DMTrCl (4.30 g, 12.68 mmol) in DCM (80 mL) was slowly added to the reaction mixture at 0° C. The reaction was stirred at rt for 1 h. The reaction mixture was quenched with saturated NaHCO₃ solution (20 mL), extracted with DCM (50 mL×2), washed with brine (50 mL), dried over Na₂SO₄, concentrated under vacuum and purified by flash column chromatography (0-3% MeOH) on Al₂O₃ to give compound 6-7 as a white foam solid (6.39 g, 80% yield).

Mass calc. for C₃₇H₃₉NO₆S: 625.25; found: 648.25 [M+Na]⁺, ESI.

¹H NMR (400 MHZ, DMSO-d₆) δ 7.68 (d, J=8.0 Hz, 2H), 7.41 (d, J=8.0 Hz, 2H), 7.33 (d, J=4.0 Hz, 2H), 7.31-7.18 (m, 7H), 6.85 (d, J=8.0 Hz, 4H), 4.68 (t, J=4.8 Hz, 1H), 3.83 (s, 4H), 3.72 (s, 6H), 3.37 (d, J=2.0 Hz, 2H), 2.94 (s, 2H), 2.39 (s, 3H), 2.07-2.03 (m, 2H), 1.92-1.89 (m, 2H).

### Compound 6-8:

Compound 6-7 (6.0 g, 9.59 mmol) was dissolved in MeOH (300 mL), then Mg (18 g) was added to the reaction mixture at 0° C. The mixture was stirred at rt overnight. The reaction mixture was quenched with H₂O, extracted with DCM, washed with brine (50 mL), dried over Na₂SO₄, concentrated under vacuum and purified by flash column chromatography (0-15% MeOH) on Al₂O₃ to give compound 6-8 as a white foam solid (1.2 g, 26.6% yield).

Mass calc. for C₃₀H₃₃NO₄: 471.24; found: 472.20 [M+H]⁺, ESI.

¹H NMR (400 MHZ, DMSO-d₆) δ 7.37 (d, J=8.0 Hz, 2H), 7.32-7.28 (m, 2H), 7.25-7.19 (m, 5H), 6.89-6.87 (m, 4H), 4.76 (s, 1H), 3.83 (s, 1H), 3.73 (s, 6H), 3.47 (s, 3H), 3.45 (s, 3H), 3.00 (s, 2H), 2.13-2.09 (m, 2H), 1.97-1.93 (m, 2H).

### Compound 6-9:

Compound 6-8 (2.4 g. 5.09 mmol) and Fmoc-6-aminohexanoic acid (1.80 g, 5.09 mmol) were dissolved in DCM (60 mL), then DIEA (1.97 g, 15.27 mmol), EDCI (1.95 g, 10.18 mmol) and HOBT (1.38 g, 10.18 mmol) were added to the mixture. The reaction was stirred at rt for 2 h. The reaction mixture was quenched with saturated NaHCO₃ solution (200 mL), extracted with DCM (60 mL×3), washed with brine (60 mL), dried over Na₂SO₄, concentrated under vacuum and purified by flash column chromatography (0-10% MeOH) on Al₂O₃ to give compound 6-9 as a white foam solid (3.1 g, 75% yield).

¹H NMR (400 MHZ, DMSO-d₆) δ 7.88 (d, J=7.4 Hz, 2H), 7.68 (d, J=7.4 Hz, 2H), 7.43-7.37 (m, 4H), 7.34-7.28 (m, 5H), 7.26-7.21 (m, 5H), 6.88 (d, J=8.9 Hz, 4H), 4.75 (t, J=4.8 Hz, 1H), 4.30-4.28 (m, 2H), 4.22-4.19 (m, 1H), 4.11-3.91 (m, 2H), 3.91-3.85 (m, 2H), 3.73 (s, 6H), 3.49-3.48 (d, J=4.6 Hz, 2H), 3.02-2.95 (m, 4H), 2.20-2.14 (m, 4H), 2.00-1.97 (m, 2H), 1.52-1.46 (m, 2H), 1.44-1.36 (m, 2H), 1.24-1.29 (m, 2H).

### Compound 6-10:

Compound 6-9 (3.1 g, 3.84 mmol) was dissolved in MeOH (60 mL), then piperidine (6 mL) was added to the mixture. The reaction was stirred at rt for 8 h. The reaction mixture was quenched with water and extracted with DCM (100 mL×3), washed with brine (60 mL), dried over Na₂SO₄, concentrated under vacuum and purified by flash column chromatography (0-20% MeOH) on Al₂O₃ to give compound 6-10 as a white foam solid (1.45 g, 64% yield).

Mass calc. for C₃₆H₄₄N₂O₅: 584.33; found: 585.4 [M+H]⁺, ESI.

¹H NMR (400 MHZ, DMSO-d₆) δ 7.39-7.39 (m, 2H), 7.37-7.28 (m, 2H), 7.25-7.19 (m, 5H), 6.88 (d, J=8.8 Hz, 2H), 4.76 (s, 1H), 4.04 (m, 2H), 3.86 (m, 2H), 3.78 (s, 6H), 3.49 (s, 2H), 3.02 (s, 2H), 2.55 (m, 2H), 2.22-2.14 (m, 4H), 2.01-1.97 (m, 2H), 1.53-1.46 (m, 2H), 1.37-1.25 (m, 5H).

### Compound 6-11:

Compound 6-10 (1.5 g, 2.57 mmol) and compound 1-4 (1.15 g, 2.57 mmol) were dissolved in DCM (60 mL), then DIEA (1.66 g, 12.83 mmol), EDCI (1.10 g, 6.41 mmol), and HOBT (519.92 mg, 3.85 mmol) were added to the mixture. The reaction was stirred at rt for 2 h. The reaction mixture was quenched with saturated NaHCO₃ solution (200 mL), extracted with DCM (60 mL×3), washed with brine (60 mL), dried over Na₂SO₄, concentrated under vacuum and purified by flash column chromatography (0-10% MeOH) on Al₂O₃ to give compound 6-11 as a white foam solid (2 g, 76% yield).

Mass calc. for C₅₅H₇₁N₃O₁₅: 1013.49; found: 1014.4 [M+H]⁺, ESI.

¹H NMR (400 MHZ, DMSO-d₆) δ 7.82 (d, J=9.2 Hz, 1H), 7.72 (t, J=5.6 Hz, 1H), 7.39-7.37 (m, 2H), 7.32-7.28 (m, 2H), 7.26-7.19 (m, 5H), 6.90-6.87 (m, 4H), 5.22 (d, J=3.4 Hz, 1H), 4.97 (dd, J=11.2, 3.4 Hz, 1H), 4.50-4.48 (m, 1H), 4.03-4.00 (m, 5H), 3.91-3.84 (m, 3H), 3.74 (s, 7H), 3.49 (d, J=4.6 Hz, 1H), 3.43-3.38 (m, 1H), 3.02-2.98 (m, 4H), 2.21-2.15 (m, 4H), 2.10-2.08 (m, 3H), 2.05-2.00 (m, 7H), 1.89 (s, 3H), 1.78 (s, 3H), 1.52-1.35 (m, 9H), 1.27-1.24 (m, 3H).

### Compound 6-12:

To a solution of compound 6-11 (790.0 mg, 0.78 mmol) in anhydrous DCM (3.0 mL) was added 4,5-dicyanoimidazole (96.0 mg, 0.81 mmol) and 2-Cyanoethyl N,N,N′,N′-tetraisopropylphosphorodiamidite (324 mg, 1.08 mmol) at rt and stirred for 1 h, LCMS showed the starting material was consumed completely. The solution was quenched by water (100 mL), washed with brine (300 mL×3) and dried over by Na₂SO₄. Then the solution was concentrated under reduced pressure and the residue was purified by Flash-Prep-HPLC with the following conditions (Column, C18 silica gel; mobile phase, CH₃CN/H₂O=1/1 increasing to CH₃CN/H₂O=1/0 within 20 min; Detector, UV 254 nm). This resulted in to give 6-12 as a white solid (680 mg, 71% yield).

Mass calc. for C₆₄H₈₈N₅O₁₆P: 1213.60; found: 1159.5 [M-CH₂CH₂CN], ESI.

¹HNMR (600 MHZ, DMSO-d₆) δ 7.82-7.68 (m, 2H), 7.38-7.19 (m, 9H), 6.90-6.86 (m, 4H), 5.21 (d, J=6.0 Hz, 1H), 4.98-4.94 (m, 1H), 4.48 (d, J=12.0 Hz, 1H), 4.04-3.99 (m, 4H), 3.90-3.37 (m, 16H), 3.09-2.87 (m, 4H), 2.71-2.68 (m, 2H), 2.19-1.76 (m, 20H), 1.52-1.05 (m, 22H).

³¹PNMR (242 MHZ, DMSO-d₆) δ 146.51, 146.47.

### Example 7

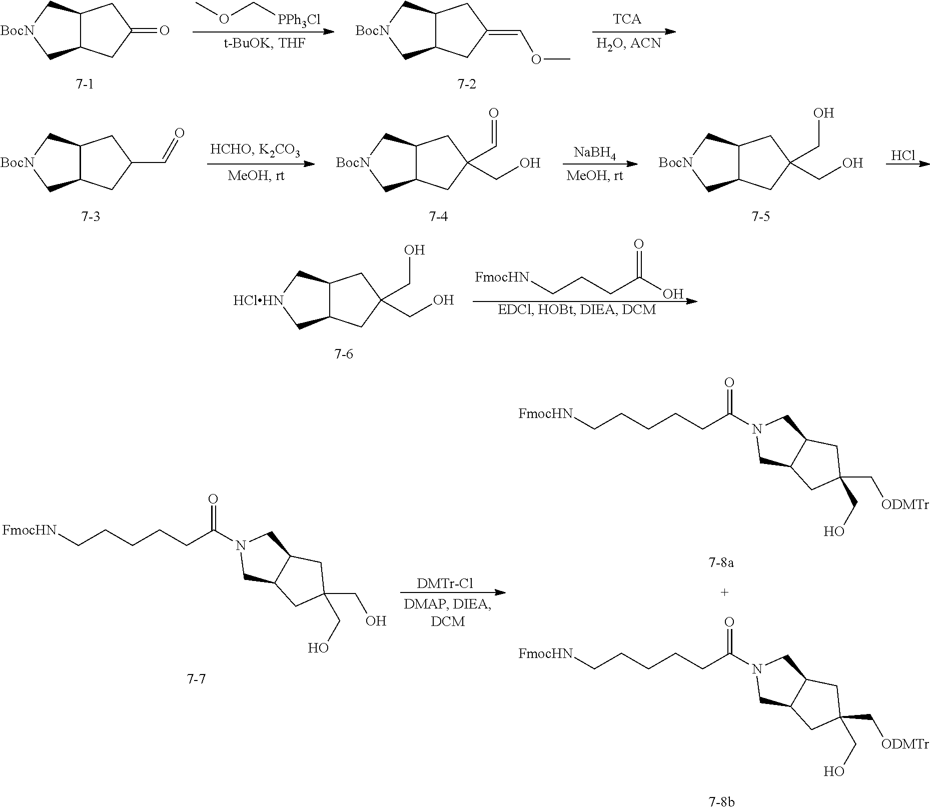

### Compound 7-2:

To a solution of methoxymethyl(triphenyl)phosphonium chloride (54.78 g, 159.80 mmol) in THF (600 mL) was added KOtBu (19.92 g, 177.55 mmol) at 0° C. and stirred for 0.5 h. Compound 7-1 (20 g, 88.78 mmol) was then added at 0° C. and stirred overnight, allowing the reaction to warm to rt. LC-MS showed full conversion. The reaction mixture was adjusted to pH6 by citric acid (10% aq.), filtered and concentrated to dryness. The residue was purified by silica gel column (0-5% EA in PE), to afford compound 7-2 as an oil (15.5 g, 68% yield).

Mass calc. for C₁₄H₂₃NO₃: 253.2; found: 239.1 [M−tBu+ACN+H]⁺, ESI.

¹H NMR (400 MHZ, DMSO-d₆) δ 5.99 (pent, J=2.1 Hz, 1H), 3.48 (s, 3H), 3.44-3.36 (m, 2H), 2.99-2.91 (m, 2H), 2.59 (br, 2H), 2.44-2.36 (m, 2H), 2.04-1.97 (m, 2H), 1.38 (s, 9H).

### Compound 7-3:

To a solution of compound 7-2 (5 g, 19.74 mmol) in MeCN (150 mL) was added 2,2,2-trichloroacetic acid (4.06 g, 24.87 mmol, in 50 mL of water). The reaction mixture was stirred at rt overnight. TLC showed full conversion. The reaction mixture was adjusted to pH7 by NaHCO₃ (aq, sat), and extracted by EA. The organic phase was concentrated to afford crude compound 7-3 as an oil (4.7 g 99% yield), which is used directly in the next step without further purification.

Mass calc. for C₁₃H₂₁NO₃: 239.2; found: 225.1 [M−tBu+ACN+H]⁺, ESI.

### Compound 7-4:

To a mixture of compound 7-3 (4.7 g, 19.64 mmol) and K₂CO₃ (542.87 mg, 3.93 mmol) in MeOH (150 mL) was added formalin (15.9 g 37% aq. formaldehyde, 55.78 mmol). The reaction mixture was stirred at rt overnight. TLC showed full conversion. The reaction mixture was adjusted to pH6 by citric acid (10% aq.), and extracted by EA. The organic phase was concentrated and purified by silica gel column (0-3% MeOH in DCM) to afford compound 7-4 as an oil (5.2 g, 98% yield).

Mass calc. for C₁₄H₂₃NO₄: 269.2; found: 170.1 [M−Boc+H]⁺, ESI.

### Compound 7-5:

To a solution of compound 7-4 (17 g, 63.12 mmol) in MeOH (190 mL) was added NaBH₄ (5.25 g, 138.86 mmol). The reaction mixture was stirred at r.t. for 1 h. LC-MS showed full conversion. The reaction mixture was adjusted to pH6 by citric acid (10% aq), filtered and extracted by DCM. The organic phase was concentrated and purified by silica gel column (0-5% MeOH in DCM) to afford compound 7-5 (14.5 g, 84% yield).

Mass calc. for C₁₄H₂₅NO₄: 271.2; found: 172.1 [M−Boc+H]⁺, ESI.

¹H NMR (400 MHZ, DMSO-d₆) δ 4.53 (t, J=5.3 Hz), 3.37-3.32 (m, 2H), 3.27 (d, J=5.3 Hz, 2H), 3.19 (d, J=5.3 Hz, 2H), 3.07 (dd, J=10.78, 3.5 Hz, 2H), 1.60 (dd, J=13.4, 7.9 Hz, 2H) 1.39 (s, 9H), 1.27 (dd, J=13.5, 6.3 Hz, 2H).

### Compound 7-6:

To a reaction vial charged with compound 7-5 (14.5 g, 53.44 mmol) was added HCl in dioxane (4 M, 140 mL). The reaction mixture was stirred at r.t. for 1.5 h. LC-MS showed full conversion. The reaction mixture was concentrated to afford crude compound 7-6 (11 g), which was used directly for the next step without further purification.

Mass calc. for C₉H₁₇NO₂ (free base): 171.1; found: 172.1 [M−Boc+H]⁺, ESI.

### Compound 7-7:

A mixture of Fmoc-6-aminohexanoic acid (18.38 g, 52.00 mmol), DIEA (16.80 g, 130.00 mmol, 22.64 mL), HOBT (8.43 g, 62.40 mmol) and EDCI (11.96 g, 62.40 mmol) in DCM (300 mL) was stirred at 0° C. for 30 min, then compound 7-6 (10.8 g, 52.00 mmol) was added. The reaction mixture was stirred overnight, allowing it to warm to r.t. LC-MS showed full conversion. The reaction mixture was diluted with DCM (300 mL) and washed with NaHCO₃ (aq, sat) and water. The organic phase was concentrated and purified by silica gel column (0-10% MeOH in DCM) to afford compound 7-7 as a gum (19.6 g, 74% yield).

Mass calc. for C₃₀H₃₈N₂O₅: 506.3; found: 507.3 [M+H]⁺, ESI.

¹H NMR (400 MHZ, DMSO-d₆) δ 7.90-7.68 (m, 4H), 7.44-7.33 (m, 5H), 4.55 (s, 2H), 4.29 (d, J=6.8 Hz, 2H), 4.23-4.18 (m, 1H), 3.28-3.21 (m, 6H), 2.97-2.96 (m, 2H), 2.61-2.53 (m, 2H), 2.17 (t, J=7.6 Hz, 2H), 1.63-1.23 (m, 12H).

### Compound 7-8:

To a solution of compound 7-7 (19 g, 37.50 mmol), N, N-dimethylpyridin-4-amine (458.17 mg, 3.75 mmol) and DIEA (7.27 g, 56.25 mmol, 9.80 mL) in DCM (590 mL) was added DMTrCl (13.98 g, 41.25 mmol) portion-wise at 0° C. The reaction mixture was stirred overnight. LC-MS showed partial conversion, formation of 2 isomers and di-DMTr by-product. The reaction mixture was concentrated and purified by silica gel column (20-100% EA with 0.2% TEA in PE) to afford compound 7-8a and 7-8b as a mixture of endo- and exo-isomers (17.9 g, 47% yield).

Isomers were separated by prep-HPLC (C18 column, water/ACN) to afford compound 7-8a (exo-, 7.5 g) and compound 7-8b (endo-, 5.7 g).

7-8a:

Mass calc. for C₅₁H₅₆N₂O₇: 808.4; found: 832.3 [M+Na]⁺, ESI.

¹H NMR (400 MHZ, DMSO-d₆) δ 7.90-7.88 (m, 2H), 7.69-7.67 (m, 2H), 7.43-8.18 (m, 13H), 6.88-6.85 (m, 4H), 4.62 (br, 1H), 4.37-4.28 (m, 2H), 4.20 (t, J=6.7 Hz, 1H), 3.73 (s, 6H), 3.47 (dd, J=9.9, 8.2 Hz, 1H), 3.28 (br, 2H), 3.11 (td, J=12.0, 4.3 Hz, 2H), 2.99-2.91 (m, 4H), 2.66-2.54 (m, 2H), 2.19-2.06 (m, 2H), 1.67 (pent, J=6.8 Hz, 2H), 1.51-1.17 (m, 10H). 7-8b:

Mass calc. for C₅₁H₅₆N₂O₇: 808.4; found: 832.3 [M+Na]⁺, ESI.

¹H NMR (400 MHZ, DMSO-d₆) δ 7.90-7.84 (m, 4H), 7.44-7.20 (m, 13H), 6.91-6.89 (m, 4H), 6.66 (br, 1H), 6.29 (s, 2H), 3.74 (s, 6H), 3.36-3.31 (m, 3H), 3.22-3.12 (m, 3H), 2.90 (dd, J=12.7, 6.5 Hz, 2H), 2.86 (s, 2H), 2.44-2.29 (m, 2H), 2.15 (t, J=7.1 Hz, 2H), 1.64-1.18 (m, 12H).

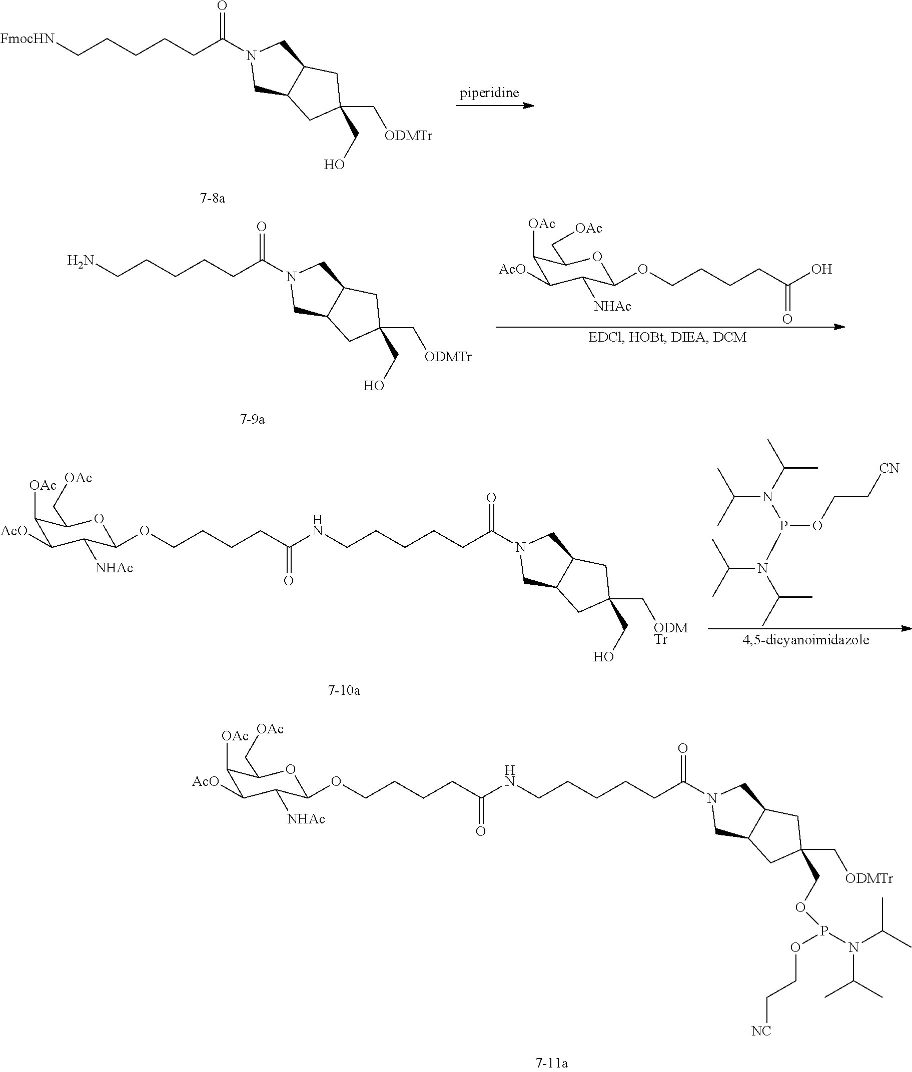

### Compound 7-9a:

A solution of compound 7-8a (6.5 g, 8.03 mmol) in MeOH (200 mL) and piperidine (20 mL) was stirred at r.t. overnight, LC-MS showed full conversion. The reaction mixture was concentrated, and the residue was purified by silica gel column (20-30% MeOH with 1% TEA in DCM) to afford compound 7-9a (4.4 g, 94% yield).

Mass calc. for C₃₆H₄₆N₂O₅: 586.3; found: 587.3 [M+H]⁺, ESI.

### Compound 7-10a:

A solution of compound 1-4 (4.70 g, 10.50 mmol), DIEA (2.42 g, 18.75 mmol, 3.27 mL), HOBT (1.22 g, 9.00 mmol) and EDCI (1.73 g, 9.00 mmol) in DCM (132 mL) was stirred at 0° C. for 30 min, then compound 7-9a (4.4 g, 7.50 mmol) was added. The reaction mixture was stirred overnight. LC-MS showed full conversion. The reaction mixture was diluted with DCM (150 mL), washed with water. The organic phase was concentrated and purified by silica gel column (0-10% MeOH with 1% TEA in DCM) to afford compound 7-10a (3.8 g, 51% yield).

Mass calc. for C₅₅H₇₃N₃O₁₅: 1015.5; found: 1039.1 [M+Na]⁺, ESI.

¹H NMR (400 MHZ, DMSO-d₆) δ 7.85 (d, J=9.2 Hz, 1H), 7.74 (t, J=5.3 Hz, 1H), 7.38-7.36 (m, 2H), 7.31-7.27 (m, 2H), 7.24-7.19 (m, 5H), 6.88-6.86 (m, 4H), 5.22 (d, J=2.8 Hz, 1H), 4.97 (dd, J=11.2, 2.8 Hz, 1H), 4.65 (t, J=4.8 Hz, 1H), 4.49 (d, J=8.4 Hz, 1H), 4.03 (s, 3H), 3.88 (dd, J=19.5, 9.1 Hz, 1H), 3.73-3.70 (m, 7H), 3.50-3.39 (m, 2H), 3.34-3.31 (m, 1H), 3.27 (d, J=4.5 Hz, 2H), 3.17 (d, J=5.2 Hz, 1H), 3.15-3.09 (m, 2H), 3.00 (dd, J=12.4, 6.3 Hz, 2H), 2.94 (br, 1H), 2.67-2.53 (m, 2H), 2.19-2.08 (m, 5H), 2.04 (t, J=6.9 Hz, 2H), 2.00 (s, 3H), 1.89 (s, 3H), 1.78 (s, 3H), 1.70-1.63 (m, 2H), 1.53-1.16 (m, 14H).

### Compound 7-11a:

To a solution of compound 7-10a (680.0 mg, 0.67 mmol) in anhydrous DCM (3.0 mL) was added 4,5-dicyanoimidazole (96.0 mg, 0.81 mmol) and 2-Cyanoethyl N,N,N′,N′-tetraisopropylphosphorodiamidite (324 mg, 1.08 mmol) at rt and stirred for 1 h, LCMS showed the starting material was consumed completely. The solution was quenched by water (100 mL), washed with brine (300 mL×3) and dried over by Na₂SO₄. Then the solution was concentrated under reduced pressure and the residue was purified by Flash-Prep-HPLC with the following conditions (Column, C18 silica gel; mobile phase, CH₃CN/H₂O=1/1 increasing to CH₃CN/H₂O=1/0 within 20 min; Detector, UV 254 nm). This resulted in to give 7-11a as a white solid (390 mg, 47% yield).

Mass calc. for C₆₄H₉₀N₅O₁₆P: 1215.61; found: 1161.3 [M-CH₂CH₂CN], ESI.

¹HNMR (600 MHZ, DMSO-d₆) δ 7.81-7.69 (m, 2H), 7.40-7.19 (m, 9H), 6.88-6.86 (m, 4H), 5.21 (d, J=6.0 Hz, 1H), 4.98-4.94 (m, 1H), 4.49 (d, J=6.0 Hz, 1H), 4.02-3.83 (m, 4H), 3.73-3.32 (m, 18H), 3.01-2.97 (m, 6H), 2.71-2.68 (m, 4H), 2.12-1.99 (m, 18H), 1.49-1.04 (m, 24H).

³¹PNMR (242 MHZ, DMSO-d₆) δ 146.70.

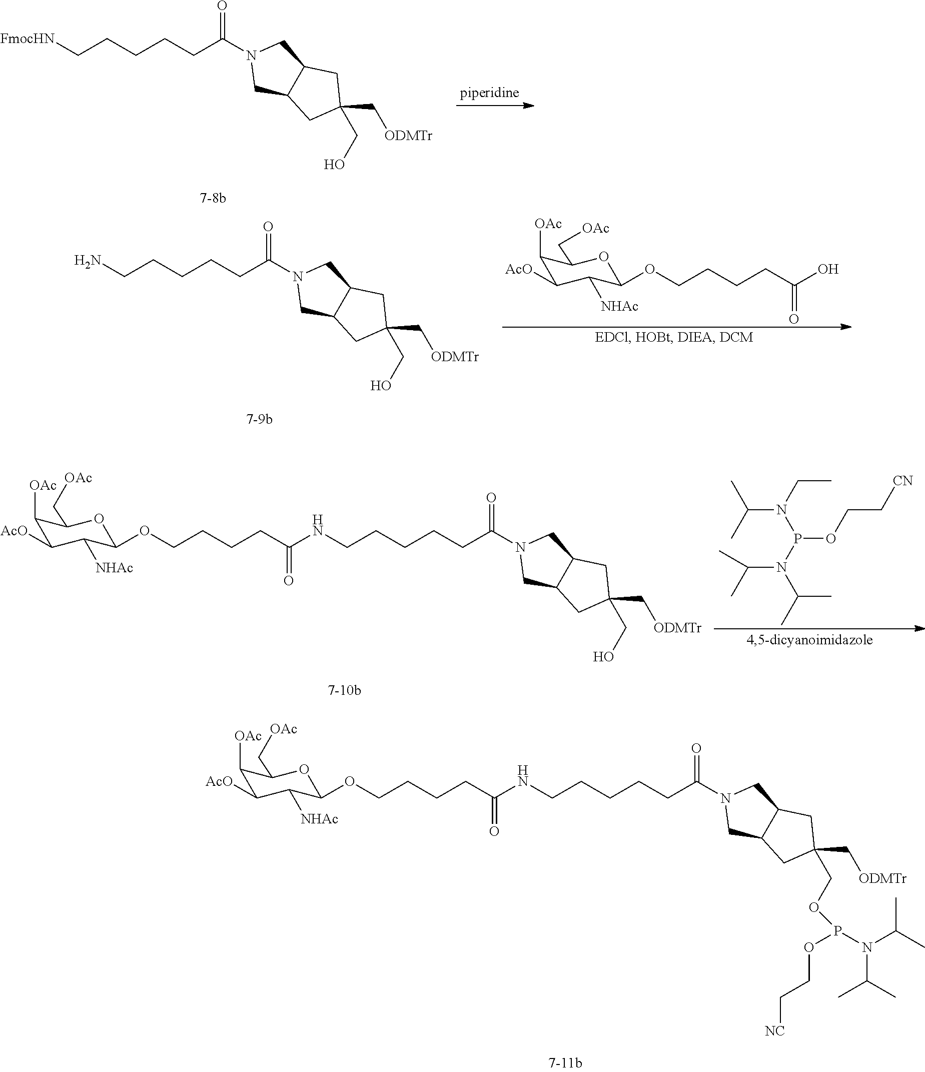

Compound 7-9b: A solution of compound 7-8b (5.70 g, 7.05 mmol) in MeOH (200 mL) and piperidine (20 mL) was stirred at r.t. overnight. LC-MS showed full conversion. The reaction mixture was concentrated and purified by silica gel column (10-20% MeOH with 1% TEA in DCM) to afford compound 7-9b (4.1 g, 99% yield).

Mass calc. for C₃₆H₄₆N₂O₅: 586.3; found: 587.3 [M+H]⁺, ESI.

### Compound 7-10b:

A solution of compound 1-4 (4.38 g, 9.78 mmol), DIEA (2.26 g, 17.47 mmol, 3.04 mL), HOBT (1.13 g, 8.39 mmol), EDCI (1.61 g, 8.39 mmol) in DCM (120 ml) was stirred at 0° C. for 30 min, then compound 7-9b (4.10 g, 6.99 mmol) was added. The reaction mixture was stirred overnight. LC-MS showed full conversion. The reaction mixture was diluted with DCM (150 mL), washed by water. The organic phase was concentrated and purified by silica gel column (0-10% MeOH with 1% TEA in DCM) to give crude product 4.4 g, which is further purified by prep-HPLC (C18, water/ACN) to afford compound 7-10b (2.0 g, 28% yield).

Mass calc. for C₅₅H₇₃N₃O₁₅: 1015.5; found: 1039.1 [M+Na]⁺, ESI.

¹H NMR (400 MHZ, DMSO-d₆) δ 7.84 (d, J=9.2 Hz, 1H), 7.72 (t, J=5.5 Hz, 1H), 7.40-7.38 (m, 2H), 7.33-7.29 (m, 2H), 7.28-7.20 (m, 5H), 6.91-6.88 (m, 4H), 5.22 (d, J=3.3 Hz, 1H), 4.97 (dd, J=11.2, 3.3 Hz, 1H), 4.66 (t, J=4.9 Hz, 1H), 4.49 (d, J=8.5 Hz, 1H), 4.03 (s, 3H), 3.88 (dd, J=20.2, 8.8 Hz, 1H), 3.74-3.69 (m, 7H), 3.48-3.38 (m, 4H), 3.34-3.31 (m, 1H), 3.21 (dd, J=10.4, 4.2 Hz, 1H), 3.14 (dd, J=12.0, 4.6 Hz, 1H), 3.00 (dd, J=12.7, 6.6 Hz, 2H), 2.86 (s, 2H), 2.46-2.27 (m, 2H), 2.16 (t, J=6.7 Hz, 2H), 2.11 (s, 3H), 2.04 (t, J=7.0 Hz, 2H), 2.00 (s, 3H), 1.89 (s, 3H), 1.78 (s, 3H), 1.63-1.12 (m, 14H).

### Compound 7-11b:

To a solution of compound 7-10b (680.0 mg, 0.67 mmol) in anhydrous DCM (3.0 mL) was added 4,5-dicyanoimidazole (96.0 mg, 0.81 mmol) and 2-Cyanoethyl N,N,N′,N′-tetraisopropylphosphorodiamidite (324 mg, 1.08 mmol) at rt and stirred for 1 h, LCMS showed the starting material was consumed completely. The solution was quenched by water (100 mL), washed with brine (300 mL×3) and dried over by Na₂SO₄. Then the solution was concentrated under reduced pressure and the residue was purified by Flash-Prep-HPLC with the following conditions (Column, C18 silica gel; mobile phase, CH₃CN/H₂O=1/1 increasing to CH₃CN/H₂O=1/0 within 20 min; Detector: UV 254 nm). This resulted in to give 7-11b as a white solid (390 mg, 47% yield).

Mass calc. for C₆₄H₉₀N₅O₁₆P: 1215.61; found: 1161.6 [M-CH₂CH₂CN], ESI.

¹HNMR (600 MHZ, DMSO-d₆) δ 7.81-7.69 (m, 2H), 7.40-7.19 (m, 9H), 6.88-6.86 (m, 4H), 5.21 (d, J=6.0 Hz, 1H), 4.98-4.94 (m, 1H), 4.49 (d, J=6.0 Hz, 1H), 4.02-3.83 (m, 4H), 3.73-3.32 (m, 18H), 3.01-2.97 (m, 6H), 2.71-2.68 (m, 4H), 2.12-1.99 (m, 18H), 1.49-1.04 (m, 24H).

³¹PNMR (242 MHZ, DMSO-d₆) δ 146.22.

### Example 8

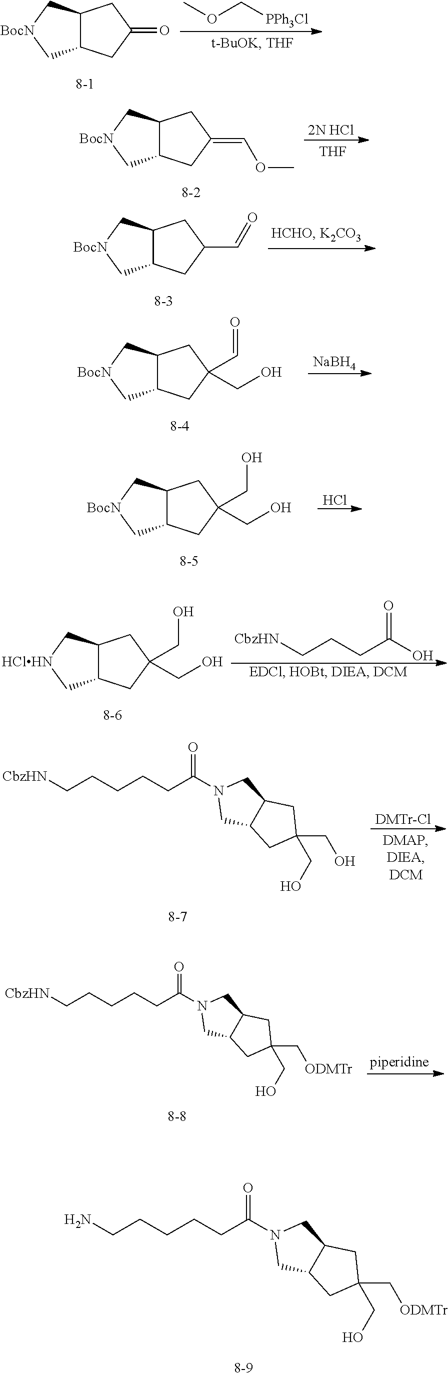

### Compound 8-2:

To a solution of methoxymethyl(triphenyl)phosphonium chloride (17.71 g, 51.67 mmol) in THF (120 mL) was added KOtBu (9.66 g, 86.12 mmol) at 0° C. and stirred for 0.5 h. Compound 8-1 (9.7 g, 43.06 mmol) was then added at 0° C. and stirred overnight, allowing the reaction to warm to rt. LC-MS showed full conversion. The reaction mixture was adjusted to pH6 by adding citric acid (10% aq.), filtered and concentrated to dryness. The residue was purified by silica gel column (0-5% EA in PE) to afford compound 8-2 as an oil (6.0 g, 55% yield).

¹H NMR (400 MHZ, DMSO-d₆) δ 6.01 (s, 1H), 3.48 (s, 3H), 3.43-3.33 (m, 2H), 2.82-2.74 (m, 2H), 2.35-2.26 (m, 2H), 1.90-1.79 (m, 4H), 1.38 (s, 9H).

### Compound 8-3:

To a solution of compound 8-2 (5.85 g, 23.09 mmol) in THF (50 mL) was added hydrochloric acid (3.28 g, 90.00 mmol, 2N, 45 mL). The reaction mixture was stirred at rt for 3 h. TLC showed full conversion. The reaction mixture was adjusted to pH7 by adding NaHCO₃ (aq., sat.), and extracted by EA. The organic phase was concentrated to afford crude compound 8-3 as an oil (5.5 g), which is used directly in the next step without further purification.

¹H NMR (400 MHZ, DMSO-d₆) δ 9.62 (pent, J=1.2 Hz, 1H), 3.42-3.33 (m, 3H), 2.82-2.72 (m, 2H), 2.03-1.93 (m, 1H), 1.92-1.87 (m, 2H), 1.75-1.62 (m, 1H), 1.51-1.44 (m, 1H), 1.38 (s, 9H).

### Compound 8-4:

To a mixture of compound 8-3 (5.25 g, 21.94 mmol) and K₂CO₃ (660.39 mg, 4.39 mmol) in MeOH (30 mL) was added formalin (6.59 g, 37% aq., 219.38 mmol). The reaction mixture was stirred at rt overnight. TLC showed full conversion. The reaction mixture was adjusted to pH6 by adding citric acid (10% aq.) and extracted by EA. The organic phase was concentrated to afford compound 8-4 (6.7 g), which was used directly in the next step without further purification.

### Compound 8-5:

To a solution of compound 8-4 (6.3 g, 23.39 mmol) in MeOH (50 mL) was added NaBH₄ (2.65 g, 70.17 mmol). The reaction mixture was stirred at rt for 1 h. LC-MS showed full conversion. The reaction mixture was adjusted to pH6 by adding citric acid (10% aq.), filtered and extracted by DCM. The organic phase was concentrated and purified by silica gel column (0-5% MeOH in DCM) to afford compound 8-5 (1.4 g, 22% yield).

Mass calc. for C₁₄H₂₅NO₄: 271.18; found: 257.24 [M−tBu+CH₃CN+H]⁺, ESI.

¹H NMR (400 MHZ, DMSO-d₆) δ 7.16-7.55 (m, 1H), 4.52-4.48 (m, 2H), 3.36-3.27 (m, 5H), 2.74-2.50 (m, 2H), 1.98-1.84 (m 2H), 1.54-1.50 (m 2H), 1.49 (s, 9H), 1.05-0.97 (m, 2H).

### Compound 8-6:

Compound 8-5 (1.35 g, 4.97 mmol) was dissolved in DCM (5 mL), HCl/Dioxane (4M, 10 mL) was added to the mixture at rt and then stirred at rt for 2 h. The reaction mixture concentrated under vacuum to give crude product 8-6 (1.3 g) as an oil, which was used in the next step without further purification.

Mass calc. for CH₁₇NO₂: 171.1; found: 172.2 [M+H]⁺, ESI.

### Compound 8-7:

Z-6-aminohexanoic acid (1.99 g, 7.51 mmol), HATU (4.72 g, 12.52 mmol) and DIPEA (2.43 g, 18.78 mmol, 3.27 mL) were dissolved in DCM (20 mL). The reaction was stirred for 20 min, then compound 8-6 (1.3 g, 6.26 mmol) was added and stirred for another 2 hours. The reaction mixture was quenched with H₂O (20 mL), washed with saturated NaHCO₃ aq. (30 mL), washed with brine (30 mL), dried over Na₂SO₄, concentrated under vacuum and purified by flash column chromatography (DCM:MeOH=20:1) on silica gel to give compound 8-7 as a white foam solid (1.5 g, 3.58 mmol, 57% yield). Mass calc. for C₂₃H₃₄N₂O₅: 418.3; found: 419.3 [M+H]⁺, ESI.

### Compound 8-8:

Compound 8-7 (1.2 g, 2.87 mmol) was dissolved in DCM (20 mL). DMTrCl (971.49 mg, 2.87 mmol) and DIPEA (741.12 mg, 5.73 mmol, 998.81 μL) were added. The reaction mixture was stirred at rt for 2 hours. Then the reaction was quenched with water, extracted with DCM (50 mL×2), washed with brine (50 mL). The organic phase was collected, dried, concentrated under vacuum and purified by flash column chromatography (DCM:MeOH:TEA=20:1:0.5%) on Al₂O₃ to give compound 8-8 as a white foam solid (850 mg, 41% yield).

Mass calc. for C₄₄H₅₂N₂O₇: 720.4; found: 721.4 [M+H]⁺, ESI.

¹H NMR (400 MHZ, DMSO-d₆) δ 7.41-7.36 (m, 3H), 7.35-7.29 (m, 6H), 7.27-7.20 (m, 5H), 6.89 (d, J=8.9 Hz, 4H), 5.00 (s, 2H), 4.67 (t, J=4.9 Hz, 1H), 3.75 (s, 6H), 3.55-3.47 (m, 2H), 3.39 (d, J=4.5 Hz, 2H), 3.00-2.95 (m, 4H), 2.92-2.84 (m, 1H), 2.62 (q, J=11 Hz, 1H), 2.16-2.10 (m, 2H), 1.95-1.70 (m, 2H), 1.62-1.58 (m, 1H), 1.51-1.35 (m, 5H), 1.27-1.23 (m, 3H), 1.12-1.00 (m, 2H).

### Compound 8-9:

Compound 8-8 (0.8 g, 1.11 mmol) was dissolved in MeOH (10 mL). Pd/C (80 mg) was added. The reaction mixture was stirred at rt for 3 h under hydrogen atmosphere. The reaction mixture was filtered and concentrated under vacuum to give crude product 8-9 as a white foam solid (550 mg, 84% yield), which was used in the next step without further purification.

Mass calc. for C₃₆H₄₆N₂O₅: 586.3; found: 587.3 [M+H]⁺, ESI.

¹H NMR (400 MHZ, DMSO-d₆) δ 7.40-7.38 (m, 2H), 7.31 (t, J=7.7 Hz, 2H), 7.29-7.18 (m, 5H), 6.89 (d, J=8.9 Hz, 4H), 3.74 (s, 6H), 3.54-3.43 (m, 2H), 3.34-3.21 (m, 2H), 2.96 (s, 2H), 2.94-2.85 (m, 2H), 2.67-2.55 (m, 1H), 2.17-2.10 (m, 2H), 1.99-1.74 (m, 2H), 1.63-1.58 (m, 1H), 1.53-1.42 (m, 3H), 1.36-1.24 (m, 5H), 1.16-1.10 (m, 1H), 1.05-0.98 (m, 1H).

### Example 9

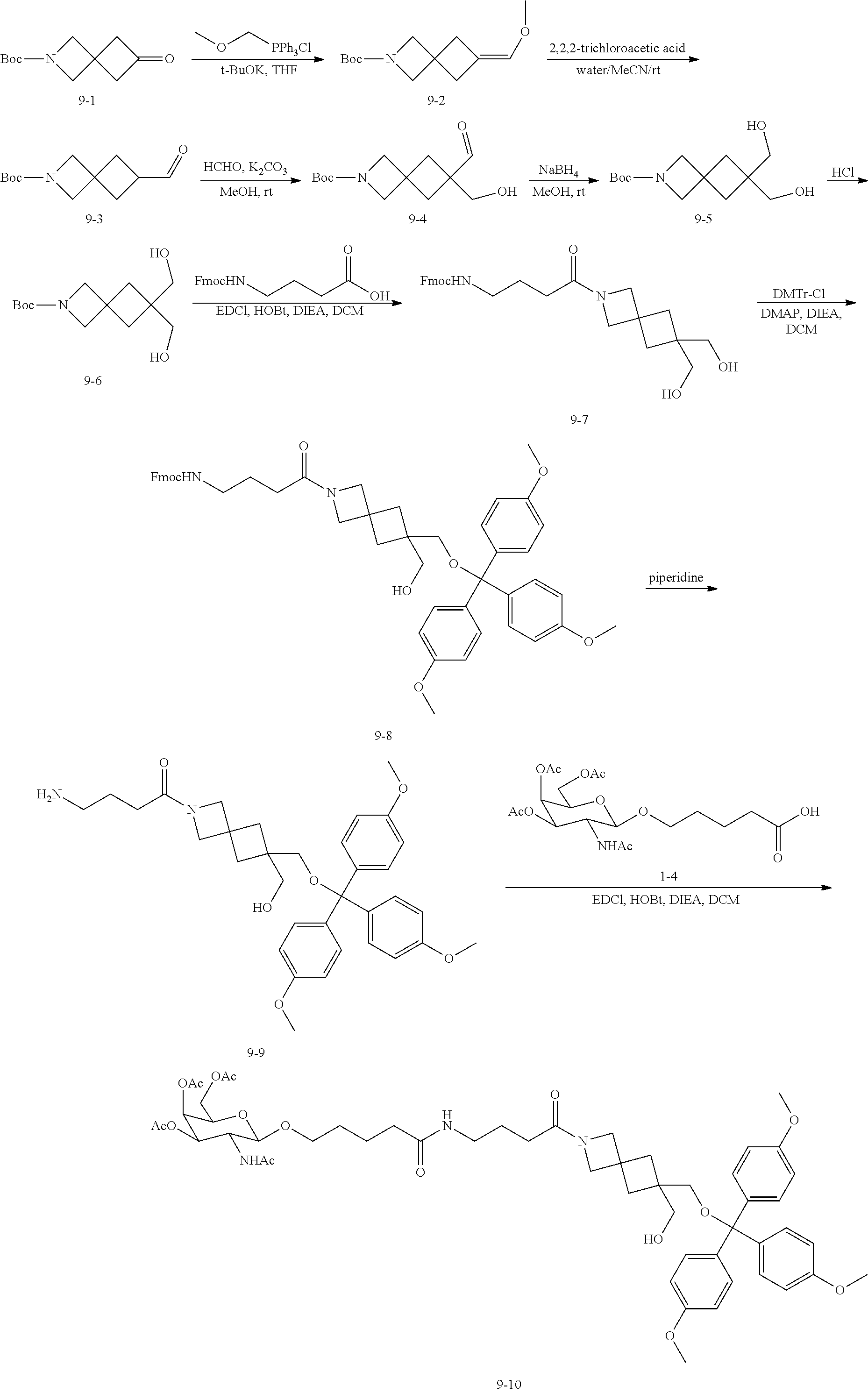

### Compound 9-2:

Methoxymethyl(triphenyl)phosphonium chloride (116.8 g, 340.72 mmol) was dissolved in THF (1200 mL). KOtBu (46.74 g, 416.56 mmol) was added to the mixture at 0° C. The reaction mixture was stirred at 0° for another 30 min. Compound 9-1 (40 g, 189.34 mmol) was added to the mixture. The mixture was warmed to rt and stirred at rt overnight. The reaction was quenched with saturated citric acid and pH was adjusted to 6-7. The reaction mixture was extracted with EA (500 mL×3), washed with brine (200 mL), dried over Na₂SO₄, concentrated under vacuum and purified by flash column chromatography (10-30% EA in PE) on silica gel to get compound 9-2 as an oil (24 g, 52% yield).

Mass calc. for C₁₃H₂₁NO₃: 239.3; found: 225.1 [M−tBu+ACN+H]⁺, ESI.

¹H NMR (400 MHZ, DMSO-d₆) δ 5.88 (t, J=2.4 Hz, 1H), 3.83 (s, 4H), 3.47 (s, 3H), 2.79-2.74 (m, 4H), 1.41 (s, 9H).

### Compound 9-3:

Compound 9-2 (15 g, 62.68 mmol) was dissolved in ACN (450 mL). 2,2,2-trichloroacetic acid (12.90 g, 78.98 mmol) in water (150 mL) was added to the reaction mixture. The reaction was stirred at rt overnight, then it was quenched with saturated NaHCO₃ and PH was adjusted to 7-8. The mixture was extracted with EA (200 mL×3), washed with brine (100 mL), dried over Na₂SO₄, concentrated under vacuum TO give crude product 9-3 as an oil (13 g, 92% yield), which was used in the next step without further purification.

Mass calc. for C₁₂H₁₉NO₃: 225.3; found: 211.1 [M−tBu+ACN+H]⁺, ESI.

### Compound 9-4:

Compound 9-3 (13 g, 57.71 mmol) was dissolved in MeOH (400 mL). K₂CO₃ (1.60 g, 11.54 mmol) and formaldehyde (4.92 g, 163.88 mmol, in water) was added to the mixture. The reaction was stirred at rt overnight, then it was quenched with saturated citric acid and PH was adjusted to 6-7. The mixture was concentrated under vacuum to remove MeOH. The residue was extracted with EA (200 mL×3), washed with brine (100 mL), dried over Na₂SO₄, concentrated under vacuum to give crude product 9-4 (14 g, 95% yield).

Mass calc. for C₁₃H₂₁NO₄: 255.3; found: 241.1 [M−tBu+ACN+H]⁺, ESI.

### Compound 9-5:

Compound 9-4 (14 g, 54.84 mmol) was dissolved in MeOH (420 mL). NaBH₄ (4.56 g, 120.64 mmol) was added portion-wise. The reaction was stirred at rt for 1 h, then it was quenched with water. PH was adjusted to 6-7 with saturated citric acid. The mixture was concentrated under vacuum to remove MeOH, then the residue was extracted with DCM (200 mL×3), washed with brine (100 mL), dried over Na₂SO₄, concentrated under vacuum and purified by flash column chromatography (0-5% MeOH) on silica gel to give compound 9-5 (7.05 g, 50% yield).

Mass calc. for C₁₃H₂₃NO₄: 257.3; found: 243.1 [M−tBu+ACN+H]⁺, ESI.

¹H NMR (400 MHZ, DMSO-d₆) δ 4.51-4.11 (m, 2H), 3.83-3.75 (m, 4H), 3.25-3.17 (m, 4H), 2.01-1.91 (m, 4H), 1.36 (s, 9H).

### Compound 9-6:

Compound 9-5 (13.1 g, 50.91 mmol) was dissolved in HCl in 1,4-Dioxane (4 M, 131 mL). The reaction mixture was stirred at rt for 1 h, then it was concentrated under vacuum to give crude product 9-6 (9.8 g), which was used in the next step without further purification.

Mass calc. for C₈H₁₃NO₂Cl: 157.2; found: 158.2 [M+H]⁺, ESI.

### Compound 9-7:

Compound 9-6 (9.8 g, 50.60 mmol) was dissolved in DCM (300 mL). DIEA (22.89 g, 177.10 mmol, 30.85 mL), HOBT (8.20 g, 60.72 mmol), EDCI (11.64 g, 60.72 mmol) and Fmoc-6-hexanoic acid (17.88 g, 50.60 mmol) were added to the reaction mixture. And the reaction was stirred at rt overnight, then it was quenched with water, washed with 10% citric acid (50 mL), saturated NaHCO₃ solution (50 mL), brine (50 mL), dried over Na₂SO₄, concentrated under vacuum and purified by flash column chromatography (0-10% MeOH) on silica gel to give compound 9-7 (6.7 g, 25% yield over two steps).

Mass calc. for C₃₀H₃₆N₂O₅: 492.3; found: 493.3 [M+H]⁺, ESI.

¹H NMR (400 MHZ, DMSO-d₆) δ 7.91-7.68 (m, 4H), 7.44-7.28 (m, 4H), 6.76 (s, 1H), 6.68-6.66 (m, 1H), 6.29 (s, 2H), 4.01 (s, 2H), 3.74 (s, 2H), 3.69 (s, 2H), 3.27 (s, 4H), 2.92-2.87 (m, 2H), 1.98-1.91 (m, 4H), 1.46-1.18 (m, 6H).

### Compound 9-8:

Compound 9-7 (6.7 g, 13.60 mmol), DMAP (166.15 mg, 1.36 mmol) and DIEA (2.64 g, 20.40 mmol, 3.18 mL) were dissolved in DCM (180 mL). DMTrCl (5.52 g, 14.96 mmol) was added to the reaction mixture portion-wise at 0° C. The reaction was stirred at rt overnight, then it was quenched with water. The mixture was extracted with DCM (50 mL×2), washed with brine (50 mL), dried over Na₂SO₄, concentrated under vacuum and purified by flash column chromatography (50-100% EA) on Al₂O₃ to give compound 9-8 (4.3 g, 40% yield).

Mass calc. for C₅₁H₅₆N₂O₈: 824.4; found: 848.3 [M+Na]⁺, ESI.

¹H NMR (400 MHZ, DMSO-d₆) δ 7.91-7.68 (m, 4H), 7.44-7.28 (m, 4H), 7.75-7.71 (m, 6H), 6.90-6.88 (m, 6H), 6.67-6.65 (m, 1H), 6.29 (s, 2H), 3.96 (s, 1H), 3.74 (s, 9H), 3.69-3.67 (m, 2H), 3.53 (s, 1H), 3.39-3.37 (m, 2H), 2.92-2.89 (m, 4H), 2.03-1.79 (m, 8H), 1.46-1.18 (m, 6H).

### Compound 9-9:

Compound 9-8 (4.2 g, 5.09 mmol) was dissolved in MeOH (126 mL), then piperidine (12.6 mL) was added to the mixture. The reaction mixture was stirred at rt overnight, then it was concentrated under vacuum and purified by flash column chromatography (0-10% MeOH) on Al₂O₃ to give compound 9-9 as a white foam (3 g, 97% yield).

Mass calc. for C₃₆H₄₆N₂O₆: 602.3; found: 603.4 [M+H]⁺, ESI.

### Compound 9-10:

Compound 1-4 (3.2 g, 7.16 mmol) and compound 9-9 (3 g, 5.11 mmol) were dissolved in DCM (130 mL). DIEA (1.65 g, 12.78 mmol, 2.17 mL), HOBT (807.01 mg, 6.13 mmol) and EDCI (1.18 g, 6.13 mmol) were added to the mixture. The reaction was stirred at rt overnight, then it was quenched with water (40 mL), extracted with DCM (50 mL×2), washed with brine (50 mL), dried over Na₂SO₄, concentrated under vacuum and purified by HPLC to give compound 9-10 (610 mg, 12% yield).

Mass calc. for C₅₅H₇₃N₃O₁₆: 1031.5; found: 1054.5 [M+Na]⁺, ESI.

¹H NMR (400 MHZ, DMSO-d₆) δ 7.82-7.73 (m, 2H), 7.24-6.88 (m, 12H), 5.22 (s, 1H), 4.97 (d, J=10.8 Hz, 1H), 4.64-4.48 (m, 2H), 4.05-3.85 (m, 4H), 3.71-3.69 (m, 12H), 3.52 (s, 1H), 3.39-3.38 (m, 1H), 3.04-2.91 (m, 4H), 2.11-1.78 (m, 19H), 1.46-1.18 (m, 14H).

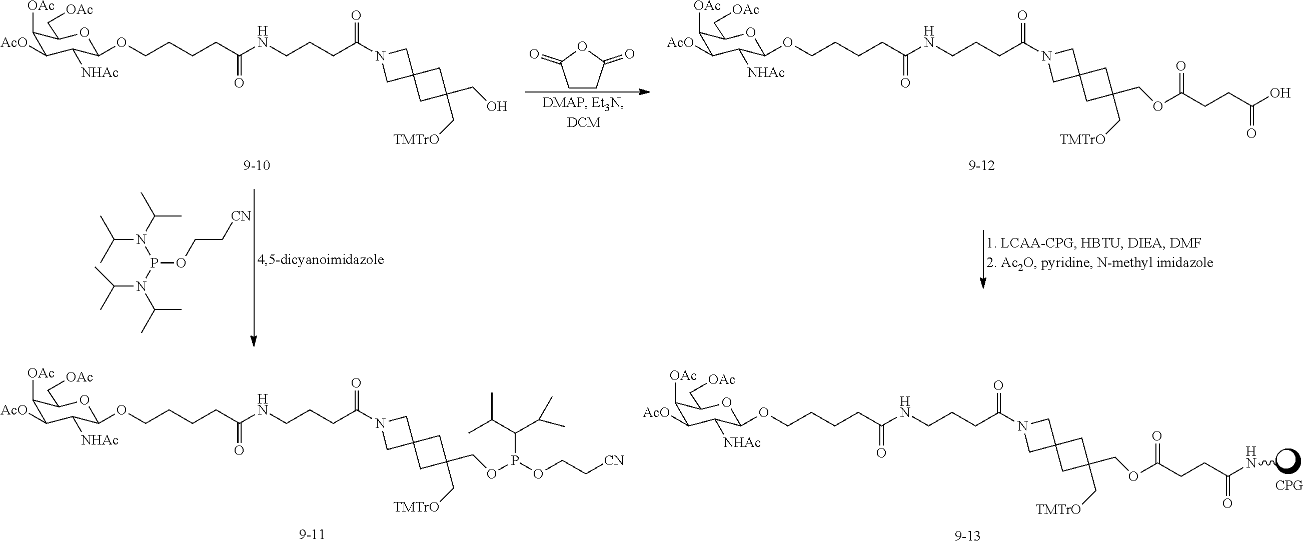

### Compound 9-11:

To a solution of compound 9-10 (1.0 g, 0.97 mmol) in anhydrous DCM (3.0 mL) was added 4,5-dicyanoimidazole (32.0 mg, 0.27 mmol) and 2-Cyanoethyl N,N,N′,N′-tetraisopropylphosphorodiamidite (108 mg, 0.36 mmol) at rt and stirred for 1 h. LCMS showed the starting material was consumed completely. The solution was quenched by water (100 mL), washed with brine (300 mL×3) and dried over by Na₂SO₄. Then the solution was concentrated under reduced pressure and the residue was purified by Flash-Prep-HPLC with the following conditions (Column: C18 silica gel; mobile phase: CH₃CN/H₂O=1/1 increasing to CH₃CN/H₂O=1/0 within 20 min; Detector: UV 254 nm). This resulted in giving compound 9-11 as a white solid (680 mg, 56% yield).

Mass calc. for C₆₄H₉₀N₅O₁₇P: 1231.61; found: 1177.1 [M-CH₂CH₂CN], ESI.

¹HNMR (600 MHZ, DMSO-d₆) δ 7.81-7.67 (m, 2H), 7.26-7.23 (m, 6H), 6.88-6.86 (m, 6H), 5.22 (d, J=6.0 Hz, 1H), 4.98-4.95 (m, 1H), 4.48 (d, J=12.0 Hz, 1H), 4.04-3.96 (m, 4H), 3.89-3.84 (m, 1H), 3.73-3.38 (m, 20H), 3.01-2.93 (m, 4H), 2.73-2.71 (m, 2H), 2.10-1.75 (m, 20H), 1.51-1.07 (m, 22H).

³¹PNMR (242 MHZ, DMSO-d₆) δ 146.82, 146.70.

### Compound 9-12:

To a solution of compound 9-10 (100 mg, 0.097 mmol) in anhydrous DCM (1.0 mL), DMAP (5 mg, 0.04 mmol) and TEA (24 mg, 0.24 mmol) were added, followed by adding succinic anhydride (20 mg, 0.2 mmol). The reaction mixture was stirred at rt for 3 h, LCMS showed the starting material was consumed completely. The reaction mixture was diluted with DCM (10 mL) and washed with H₂O (3 mL×4), then washed with brine (3 mL×4), the organic layer was concentrated to give compound 9-12 as a white solid (85 mg, 90% yield).

Mass calc. for C₅₉H₇₇N₃O₁₉: 1131.52; found: 1130.2 [M−H], ESI.

¹HNMR (600 MHZ, DMSO-d₆) δ 7.87-7.72 (m, 2H), 7.24-7.21 (m, 6H), 6.90-6.87 (m, 6H), 5.21 (d, J=6.0 Hz, 1H), 4.98-4.95 (m, 1H), 4.49 (d, J=12.0 Hz, 1H), 4.06-3.98 (m, 6H), 3.90-3.83 (m, 1H), 3.73-3.50 (m, 13H), 3.02-2.94 (m, 4H), 2.45-2.44 (m, 4H), 2.11-1.76 (m, 20H), 1.48-1.18 (m, 12H).

### Solid Support 9-13:

Native amino-LCAA-CPG (loading value: 75 umol/g, 1000 Å) was washed with ACN (100 mL×2), DMF (100 mL×2) and DCM (100 mL×2), then dried under high vacuum overnight.

To a solution of succinate 9-12 (85 mg, 0.075 mmol) and HBTU (53 mg, 0.14 mmol) in anhydrous DMF (1.5 mL), DIPEA (30 mg, 0.23 mmol) was added, the reaction mixture was shaken at rt for 10 min, then native amino-LCAA-CPG (300 mg, loading 75 umol/g) was added, the suspension was shaken at rt for 20 h, then filtered, washed with DMF (20 mL×5), ACN (20 mL×5), DCM (20 mL×5) until TLC showed no spot at 254 nm in eluent. The solid support was dried under vacuum for 2 h to give solid support (300 mg). The unreacted amines on the solid support were capped by stirring with Ac2O/pyridine/N-methyl imidazole (90 μL/1.0 mL/80 μL) at RT for 1 h, washed with DMF (20 mL×5), CAN (20 mL×5), DCM (20 mL×5) until TLC showed no spot at 254 nm in eluent. The solid support was dried under vacuum for 15 h to give solid support 9-13 (300 mg). For loading calculation, take 5.00 mg of the dried loaded CPG and add 25 mL of 3% DCA on DCM. Shake the solution and measure UV absorbance at 482 nm. Make sure the absorbance value is below 1.0 units to ensure no signal saturation. Then apply the following equation:

Loading in umol/g=(total volume of DCA added in mL)*(Abs value at 482 nm)*1000)/78.3*(mg of CPG taken)
Total volume of DCA added in mL=25 mL
Abs value at 482 nm=0.577
Mg of CPG taken=5.00 mg
Loading in umol/g=((25)*(0.577)*1000)/78.3*(5.00)=36.8 umol/g.

### Example 10

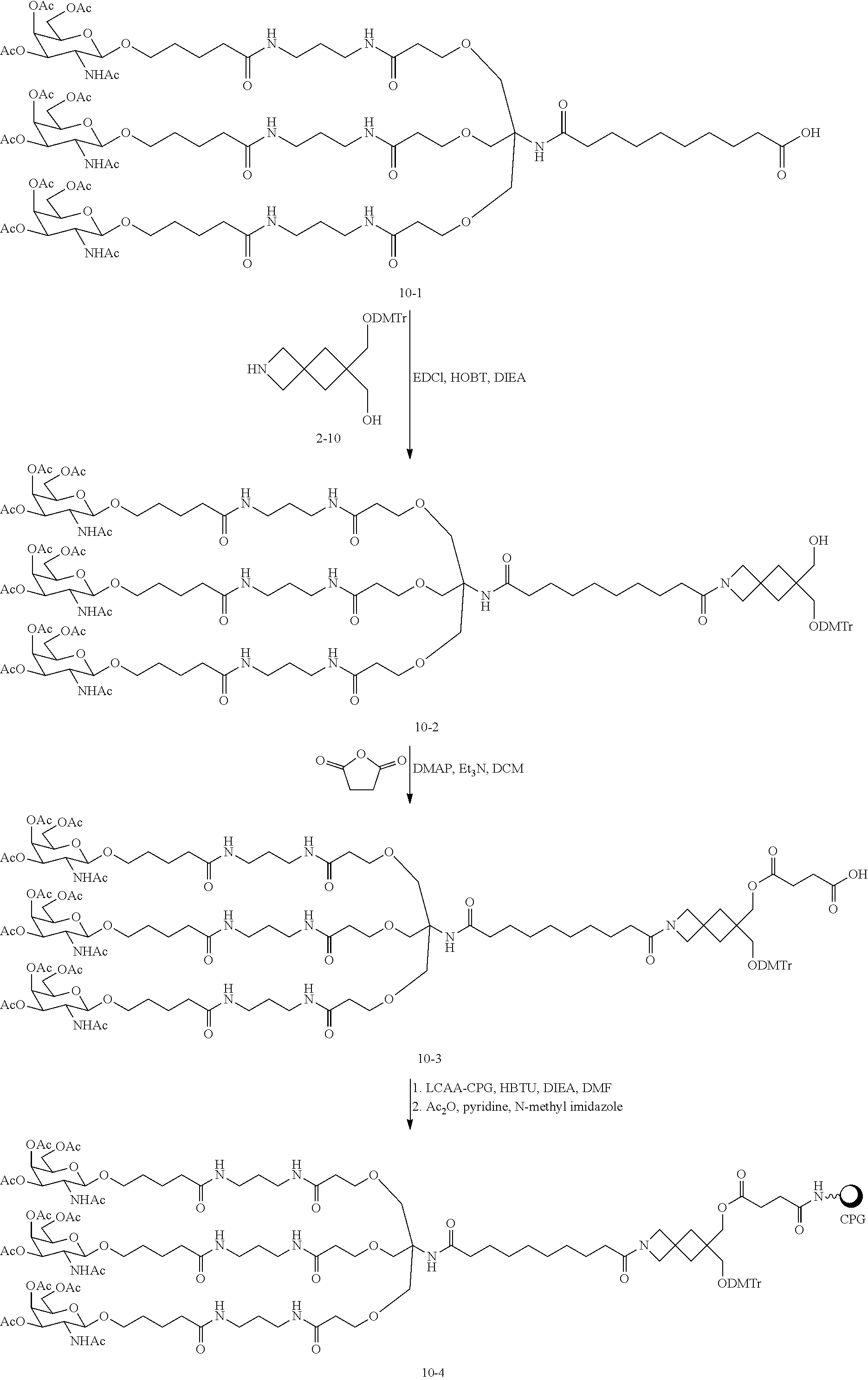

### Compound 10-2:

Compound 10-1 (0.6 g, 0.299 mmol), DIEA (96.63 mg, 0.748 mmol, 130.23 μL), HOBT (48.49 mg, 0.359 mmol) and EDCI (68.80 mg, 0.359 mmol) were mixed in DCM (18 mL) and stirred at rt for 30 min, then compound 2-10 (412.34 mg, 0.897 mmol) was added and stirred at rt for 1 h. LC-MS showed full conversion. The reaction mixture was concentrated and purified by prep-HPLC (C18 column, eluent A: water, eluent B: ACN) to afford compound 10-2 as a white solid (580 mg, 79% yield).

Mass calc. for C₁₂₀H₁₇₉N₁₁O₄₂: 2446.22; found: 1247.61 [M+2Na]²⁺, ESI.

¹H NMR (400 MHZ, DMSO-d₆) δ 7.86-7.82 (m, 6H), 7.75 (t, J=5.5 Hz, 3H), 7.39-7.29 (m, 4H), 7.26-7.19 (m, 5H), 7.00 (br, 1H), 6.89 (dd, J=8.9, 3.7 Hz, 4H), 5.21 (d, J=3.4 Hz, 3H), 4.96 (dd, J=11.2, 3.4 Hz, 3H), 4.66-4.64 (m, 1H), 4.78 (d, J=8.4 Hz, 3H), 4.04-3.96 (m, 10H), 3.90-3.83 (m, 3H), 3.73-3.67 (m, 11H), 3.55-3.50 (m, 13H), 3.43-3.37 (m, 5H), 3.02 (pent, J=5.8 Hz, 12H), 2.90 (s, 2H), 2.27 (t, J=6.2 Hz, 6H), 2.10 (s, 9H) 2.07-2.00 (m, 10H), 1.99 (s, 9H), 1.96-1.92 (m, 2H), 1.89 (s, 9H), 1.85-1.80 (m, 2H), 1.77 (s, 9H), 1.54-1.37 (m, 22H), 1.22-1.20 (m, 12H).

### Compound 10-3:

To a solution of compound 10-2 (150 mg, 0.06 mmol) in anhydrous DCM (1.0 mL), DMAP (14 mg, 0.12 mmol) and TEA (24 mg, 0.24 mmol) were added, followed by succinic anhydride (20 mg, 0.2 mmol), the reaction mixture was stirred at rt for 3 h, LCMS showed the starting material was consumed completely, the reaction mixture was diluted with DCM and washed with water (3 mL×4), then washed with brine (3 mL×4), the organic layer was concentrated to give compound 10-3 (130 mg, 97% yield) as a white solid.

Mass calc. for C₁₂₄H₁₈₃N₁₁O₄₅: 2546.24; found: 1273.5 [M+2H]²⁺, ESI.

¹HNMR (600 MHZ, DMSO-d₆): 7.84-7.73 (m, 9H), 7.37-7.21 (m, 9H), 6.98-6.89 (m, 5H), 5.21 (d, J=6.0 Hz, 3H), 4.97-4.94 (m, 3H), 4.48 (d, J=12.0 Hz, 3H), 4.07-3.66 (m, 26H), 3.55-3.37 (m, 17H), 3.04-2.95 (m, 14H), 2.45 (d, J=6.0 Hz, 4H), 2.29-2.25 (m, 6H), 2.11-1.72 (m, 50H), 1.51-1.12 (m, 38H).

### Compound 10-4:

To a solution of compound 10-3 (130 mg, 0.05 mmol) and HBTU (53 mg, 0.14 mmol) in anhydrous DMF (1.5 mL), DIPEA (30 mg, 0.23 mmol) was added. The reaction mixture was shaken at rt for 10 min, then native amino-lcaa-CPG (300 mg, loading 75 μmol/g) was added, the suspension was shaken at rt for 20 h, then filtered, washed with DMF (20 mL×5), ACN (20 mL×5), DCM (20 mL×5) until TLC showed no spot at 254 nm in eluent. The solid support was dried under vacuum for 2 h to give solid support (300 mg). The unreacted amines on the support were capped by stirring with Ac₂O/pyridine/N-methyl imidazole (90 μL/1.0 mL/80 μL) at rt for 1 h, washed with DMF (20 mL×5), ACN (20 mL×5), DCM (20 mL×5) until TLC showed no spot at 254 nm in eluent. The solid support was dried under vacuum for 15 h to give solid support 10-4 (300 mg). For loading calculation, take 5.19 mg of the dried loaded CPG and add 20 mL of 3% DCA on DCM. Shake the solution and measure UV absorbance at 500 nm. Make sure the absorbance value is below 1.0 units to ensure no signal saturation. Then apply the following equation:

Loading in μmol/g=(total volume of DCA added in mL)*(Abs value at 500 nm)*1000)/76*(mg of CPG taken)
Total volume of DCA added in mL=20 mL
Abs value at 500 nm=0.335
Mg of CPG taken=5.19 mg
Loading in μmol/g=((20)*(0.335)*1000)/76*(5.19)=17 μmol/g.

### Example 11

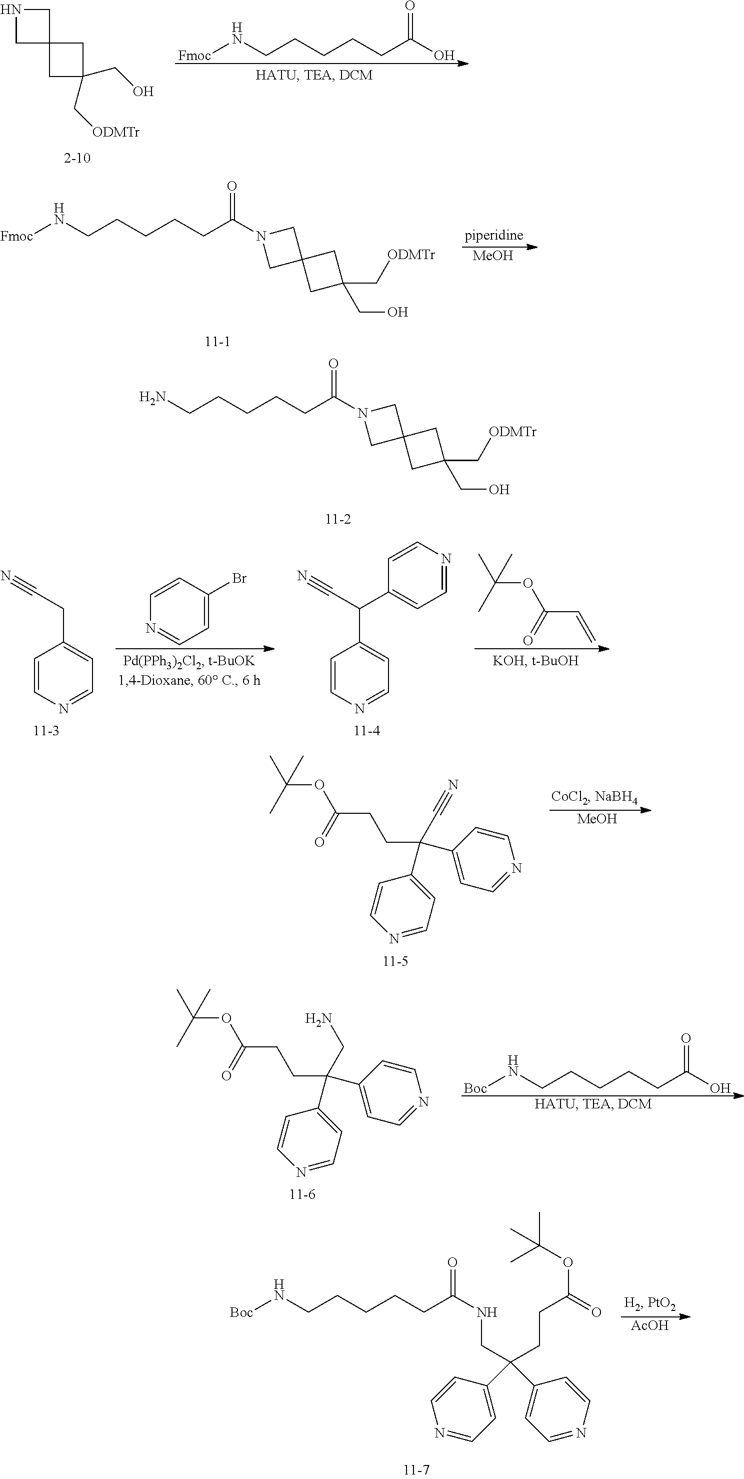

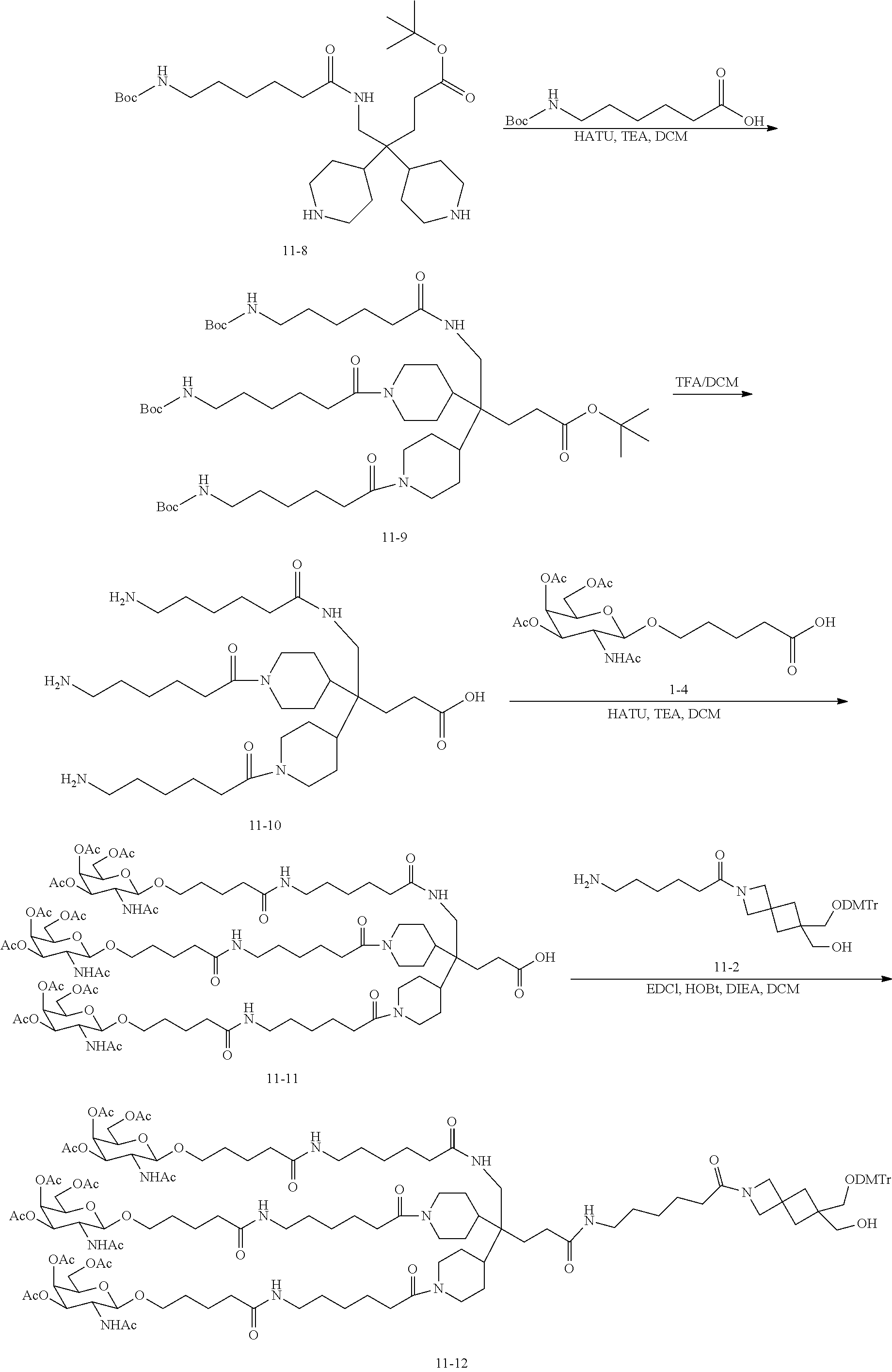

### Compound 11-1:

To a solution of compound 2-10 (1.27 g, 3.59 mmol) in DCM (15 mL) were added TEA (495.41 mg, 4.90 mmol) and HATU (1.35 g, 3.59 mmol). After stirred for 15 min, Fmoc-6-hexanoic acid (1.5 g, 3.26 mmol) was added to the reaction mixture and stirring was continued for 4 h. The mixture was partitioned between DCM (50 mL) and water (35 mL) and the DCM extract was washed with brine (100 mL), dried over sodium sulfate and concentrated in vacuo. The residue was purified by column chromatography eluting with DCM/MeOH=20/1 to give 600 mg of compound 11-1 in 23.2% yield.

¹H NMR (400 MHZ, DMSO-d₆) δ 7.87 (d, J=7.6 Hz, 2H), 7.68 (d, J=7.4 Hz, 2H), 7.44-7.35 (m, 4H), 7.35-7.28 (m, 4H), 7.27-7.17 (m, 6H), 6.88 (d, J=7.6 Hz, 4H), 4.64 (td, J=5.04, 1.6 Hz, 1H), 4.28 (d, J=6.9 Hz, 2H), 4.20 (t, J=6.8 Hz, 1H), 3.96 (s, 1H), 3.73 (d, J=3.8 Hz, 6H), 3.66 (d, J=10.4 Hz, 2H), 3.50 (s, 1H), 3.39 (t, J=5.9 Hz, 2H), 2.96 (t, J=6.6 Hz, 2H), 2.91 (s, 2H), 2.06-1.74 (m, 6H), 1.47-1.31 (m, 4H), 1.26-1.13 (m, 2H).

### Compound 11-2:

Piperidine (0.4 mL) was added to a solution of compound 11-1 (600 mg, 0.75 mmol) in MeOH (4 mL). After stirred overnight, the mixture was concentrated in vacuo. The residue was purified by column chromatography eluting with DCM/MeOH/NH₃OH=100/10/1 to give 360 mg of compound 11-2 in 82% yield.

Mass calc. for C₃₅H₄₄N₂O₅: 572.3; found: 573.4 [M+H]⁺, ESI.

¹H NMR (400 MHZ, DMSO-d₆) δ 7.27-7.21 (m, 4H), 6.91-6.85 (m, 9H), 3.97 (s, 1H), 3.73 (s, 6H), 3.68 (s, 2H), 3.47 (d, J=6.5 Hz, 2H), 3.16 (s, 2H), 2.90 (s, 2H), 2.66-2.62 (m, 2H), 2.54-2.51 (m, 2H), 1.49-1.17 (m, 12H).

### Compound 11-4:

A mixture of compound 11-3 (10 g, 84.65 mmol), 4-bromopyridine (14.71 g, 93.11 mmol), Pd(PPh₃)₂Cl₂ (4 g, 5.70 mmol) and t-BuOK (19.00 g, 169.30 mmol) in 1,4-dioxane (200 mL) was degassed by purging with nitrogen. The mixture was then heated at 60° C. for 6 hours. The mixture was concentrated. The residue was then purified by column chromatography eluting with DCM/MeOH/NH₄OH=95/5/1 to give 8 g of compound 11-4 as a yellow solid in 48% yield.

Mass calc. for C₁₂H₉N₃: 195.1; found: 196.2 [M+H]⁺, ESI.

### Compound 11-5:

Compound 11-4 (10 g, 51.22 mmol) was dissolved in tert-butanol and warmed to 50° C., then KOH (574.84 mg, 10.24 mmol and MeOH (5 mL) were added. tert-butyl acrylate (7.22 g, 56.35 mmol) was then slowly added to the reaction mixture and allowed to stir for 5 h. The mixture was concentrated in vacuo. The residue was partitioned between ethyl acetate (250 mL) and water (150 mL) and the ethyl acetate extract was washed with brine (100 ml), dried over sodium sulfate and concentrated in vacuo. The residue was purified by column chromatography eluting with PE/EA=1/2 to give 11 g of compound 11-5 in 66% yield.

Mass calc. for C₁₉H₂₁N₃O₂: 323.2; found: 324.2 [M+H]⁺, ESI.

¹H NMR (400 MHZ, DMSO-d₆) δ 8.65 (d, J=5.6 Hz, 4H), 7.50 (d, J=6.0 Hz, 4H), 2.82 (t, J=7.8 Hz, 2H), 2.23 (t, J=7.8 Hz, 2H), 1.35 (s, 9H).

### Compound 11-6:

Cobalt chloride hexahydrate (1.47 g, 6.18 mmol) was added to a solution of compound 11-5 (1 g, 3.09 mmol) in MeOH (20 mL). Sodium borohydride (1.17 g, 30.92 mmol) then was slowly added to the reaction mixture at −6° C. The mixture was allowed to warm to room temperature and stirred for 5 h. The mixture was concentrated in vacuo. The residue was dissolved in EA and filtered through a bed of celite diatomaceous earth and washed with MeOH. The filtrate was concentrated to give 0.7 g of compound 11-6 in 69% yield.

Mass calc. for C₁₉H₂₅N₃O₂: 327.2; found: 328.3 [M+H]⁺, ESI.

### Compound 11-7:

Compound 11-6 (2.12 g, 9.16 mmol) was dissolved in DCM (40 mL). Then TEA (925.42 mg, 9.16 mmol) and HATU (3.46 g, 9.16 mmol) were added to this solution. After stirred for 20 min, Boc-6-aminohexanoic acid (3 g, 9.16 mmol) was added to the reaction mixture and the mixture was stirred for 5 h. The mixture was partitioned between DCM (50 mL) and water (35 mL) and the DCM extract was washed with brine (100 mL), dried over sodium sulfate and concentrated in vacuo. The residue was purified by column chromatography eluting with DCM/MeOH=9/1 to give 2.5 g of compound 11-7 as a yellow oil in 71% yield.

Mass calc. for C₃₀H₄₄N₄O₅: 540.3; found: 541.4 [M+H]⁺, ESI.

¹H NMR (400 MHZ, DMSO-d₆) δ 8.48 (d, J=5.9 Hz, 4H), 7.45 (t, J=6.6 Hz, 1H), 7.14 (d, J=6.1 Hz, 4H), 6.73 (t, J=5.4 Hz, 1H), 3.88 (d, J=6.1 Hz, 2H), 2.85 (q, J=6.6 Hz, 2H), 2.31 (t, J=8.0 Hz, 2H), 1.94 (q, J=8.0 Hz, 4H), 1.36 (s, 9H), 1.32 (s, 9H), 1.31-1.23 (m, 4H), 1.1-0.9 (m, 2H).

### Compound 11-8:

Platinum oxide (2.2 g, 9.69 mmol) was added to a solution of compound 11-7 (4.5 g, 8.32 mmol) in AcOH (15 ml). The mixture was hydrogenated under ˜0.4 MPa for 56 hours. The catalyst was filtered through a bed of celite diatomaceous earth and washed with MeOH. The mixture was concentrated in vacuo. pH was adjusted to 7 by addition of conc. NaHCO₃ aqueous solution and then dried in vacuo. The residue was dissolved in THE, then the mixture was filtered, and the filtrate was evaporated under reduced pressure to give 4 g of compound 11-8 as a colorless oil in 87% yield.

Mass calc. for C₃₀H₅₆N₄O₅: 552.4; found: 553.4 [M+H]⁺, ESI.

### Compound 11-9:

TEA (402.72 mg, 3.98 mmol) and HATU (1.50 g, 3.98 mmol) were added to a solution of Boc-6-aminohexanoic acid (920.49 mg, 3.98 mmol) in DCM (10 mL). After stirring the solution for 20 min, compound 11-8 (1 g, 1.18 mmol) was added to the reaction mixture and stirring was continued for 5 h. The mixture was partitioned between DCM (50 mL) and water (35 mL) and the DCM extract was washed with brine (100 mL), dried over sodium sulfate and concentrated in vacuo. The residue was purified by column chromatography eluting with DCM/MeOH=15/1 to give 0.4 g of compound 11-9 as a white solid in 22% yield.

Mass calc. for C₅₂H₉₄N₆O₁₁: 978.7; found: 979.8 [M+H]⁺, ESI.

¹H NMR (400 MHZ, DMSO-d₆) δ 7.41 (t, J=6.0 Hz, 1H), 6.78-6.70 (m, 3H), 3.88 (d, J=11.4 Hz, 2H), 3.02 (d, J=6.5 Hz, 6H), 2.92-2.82 (m, 8H), 2.25 (t, J=7.4 Hz, 4H), 2.15 (t, J=8.1 Hz, 2H), 2.16 (t, J=7.2 Hz, 2H), 1.70-1.50 (m, 6H), 1.49-1.38 (m, 10H), 1.38 (s, 9H), 1.36 (s, 27H), 1.34-1.15 (m, 14H).

### Compound 11-10:

TFA (2 mL) was added to a solution of compound 11-9 (1.4 g, 1.43 mmol) in DCM (6 mL), then the mixture was stirred for 4 h at rt. The mixture was concentrated in vacuo to give 850 mg of compound 11-10 as a colorless oil in 95% yield.

Mass calc. for C₃₃H₆₂N₆O₅: 622.5; found: 623.6 [M+H]⁺, ESI.

### Compound 11-11:

TEA (584.84 mg, 5.78 mmol) and HATU (1.64 g, 4.33 mmol) were added to a solution of compound 1-4 (2.13 g, 4.77 mmol) in DMF (10 mL). After stirred for 20 min, compound 11-10 (0.9 g, 1.44 mmol) was added to the reaction mixture and stirring was continued for 5 h. The mixture was purified by reverse phase column (C18 column) to provide 1.3 g of compound 11-11 as a white solid in 47% yield.

Mass calc. for C₉₀H₁₄₃N₉O₃₅: 1909.9; found: 956.0 [M+2H]²⁺, ESI.

¹H NMR (400 MHZ, DMSO-d₆) δ 12.0 (br, 1H), 7.83 (d, J=9.2 Hz, 3H), 7.70 (t, J=5.5 Hz, 3H), 7.50-7.40 (m, 1H), 5.20 (d, J=3.4 Hz, 3H), 4.96 (dd, J=11.24, 3.4 Hz, 3H), 4.50-4.40 (m, 5H), 4.08-3.96 (m, 9H), 3.92-3.82 (m, 5H), 3.76-3.66 (m, 3H), 3.46-3.36 (m, 4H), 3.15-2.75 (m, 11H), 2.40-2.30 (m, 2H), 2.25 (t, J=7.3 Hz, 4H), 2.10 (s, 9H), 2.08-2.00 (m, 8H), 1.99 (s, 9H), 1.98 (s, 9H), 1.77 (s, 9H), 1.60-1.08 (m, 42H).

### Compound 11-12:

Compound 11-11 (1.4 g, 0.73 mmol) was dissolved in DCM (15 mL), then HOBT (148.47 mg, 1.10 mmol), EDCI (210.10 mg, 1.10 mmol) and DIEA (236.69 mg, 1.83 mmol) were added to this solution. After stirred for 15 min, compound 11-2 (503.47 mg, 0.88 mmol) was added to the reaction mixture and stirring was continued for 4 h. The mixture was partitioned between DCM (50 mL) and water (35 mL) and the DCM extract was washed with brine (100 mL), dried over sodium sulfate and concentrated in vacuo. The residue was purified by reverse phase column (C18 column) to provide 0.8 g of compound 11-12 as a white powder in 44% yield.

Mass calc. for C₁₂₅H₁₈₅N₁₁O₃₉: 2464.3; found: 1255.6 [M+2Na]²⁺, ESI.

¹H NMR (400 MHZ, DMSO-d₆) δ 7.86-7.76 (m, 4H), 7.71 (t, J=5.3 Hz, 3H), 7.45 (s, 1H), 7.41-7.35 (m, 2H), 7.34-7.28 (m, 2H), 7.27-7.18 (m, 5H), 6.93-6.86 (m, 4H), 5.27-5.15 (d, J=3.3 Hz, 3H), 5.02-4.90 (dd, J=11.2, 3.4 Hz, 3H), 4.69-4.60 (m, 1H), 4.57-4.33 (m, 5H), 4.11-3.98 (m, 9H), 3.97 (s, 1H), 3.93-3.81 (m, 5H), 3.73 (s, 6H), 3.72-3.62 (m, 5H), 3.51 (s, 1H), 3.46-3.36 (m, 5H), 3.14-2.73 (m, 14H), 2.43-2.29 (m, 2H), 2.29-2.19 (m, 4H), 2.01 (s, 9H), 2.06-1.97 (m, 19H), 1.96-1.91 (m, 3H), 1.87 (s, 9H), 1.86-1.80 (m, 2H), 1.77 (s, 9H), 1.70-1.55 (m, 5H), 1.54-1.13 (m, 44H).

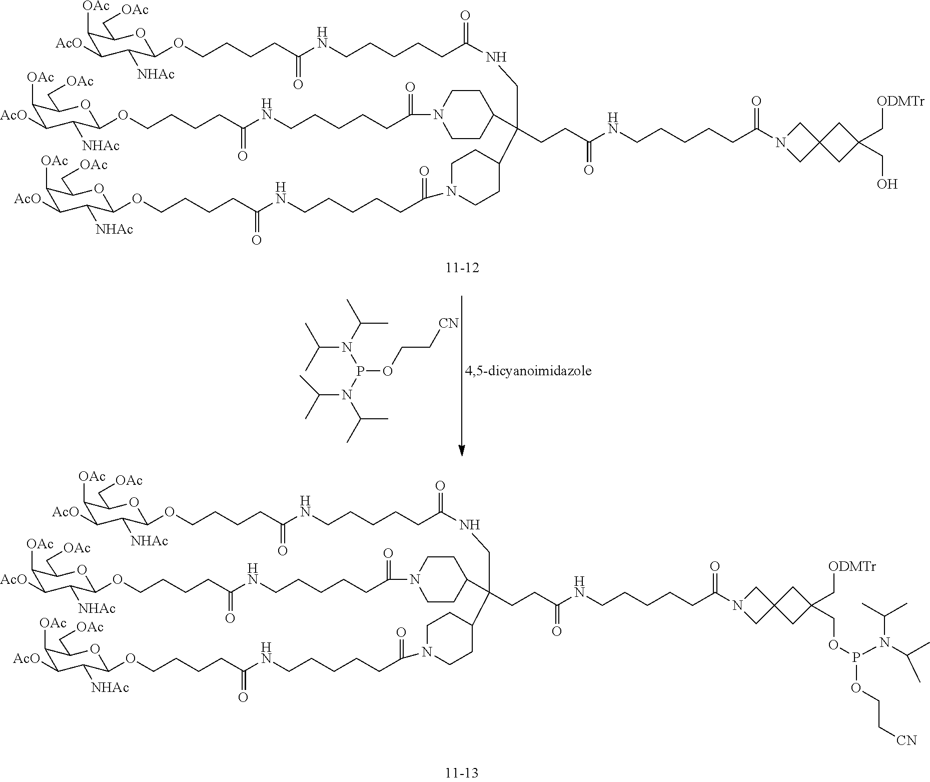

### Compound 11-13:

To a solution of compound 11-12 (2.0 g, 0.8 mmol) in anhydrous DCM (7.0 mL) was added 4,5-dicyanoimidazole (80.0 mg, 0.67 mmol) and 2-Cyanoethyl N,N,N′,N′-tetraisopropylphosphorodiamidite (270 mg, 0.90 mmol) at rt and stirred for 1 h, LCMS showed the starting material was consumed completely.

The solution was quenched by water (100 mL), washed with brine (300 mL×3) and dry over by Na₂SO₄. Then the solution was concentrated under reduced pressure and the residue was purified by Flash-Prep-HPLC with the following conditions (Column, C18 silica gel; mobile phase, CH₃CN/H₂O=1/1 increasing to CH₃CN/H₂O=1/0 within 20 min; Detector, UV 254 nm). This resulted in giving compound 11-13 as a white solid (1.0 g, 45% yield).

Mass calc. for C₁₃₄H₂₀₂N₁₃O₄₀P: 2664.39; found: 1132.5 [M-DMTr-N(CH(CH₃)₂)₂+4H]²⁺, ESI.

¹HNMR (600 MHZ, DMSO-d₆) δ 7.82-7.69 (m, 6H), 7.44-7.19 (m, 9H), 6.90-6.87 (m, 4H), 5.21 (d, J=6.0 Hz, 3H), 4.98-4.94 (m, 3H), 4.49-4.44 (m, 5H), 4.04-3.37 (m, 35H), 3.02-2.71 (m, 15H), 2.33-1.75 (m, 56H), 1.64-1.07 (m, 57H).

³¹PNMR (242 MHZ, DMSO-d₆) δ 146.79, 146.67.

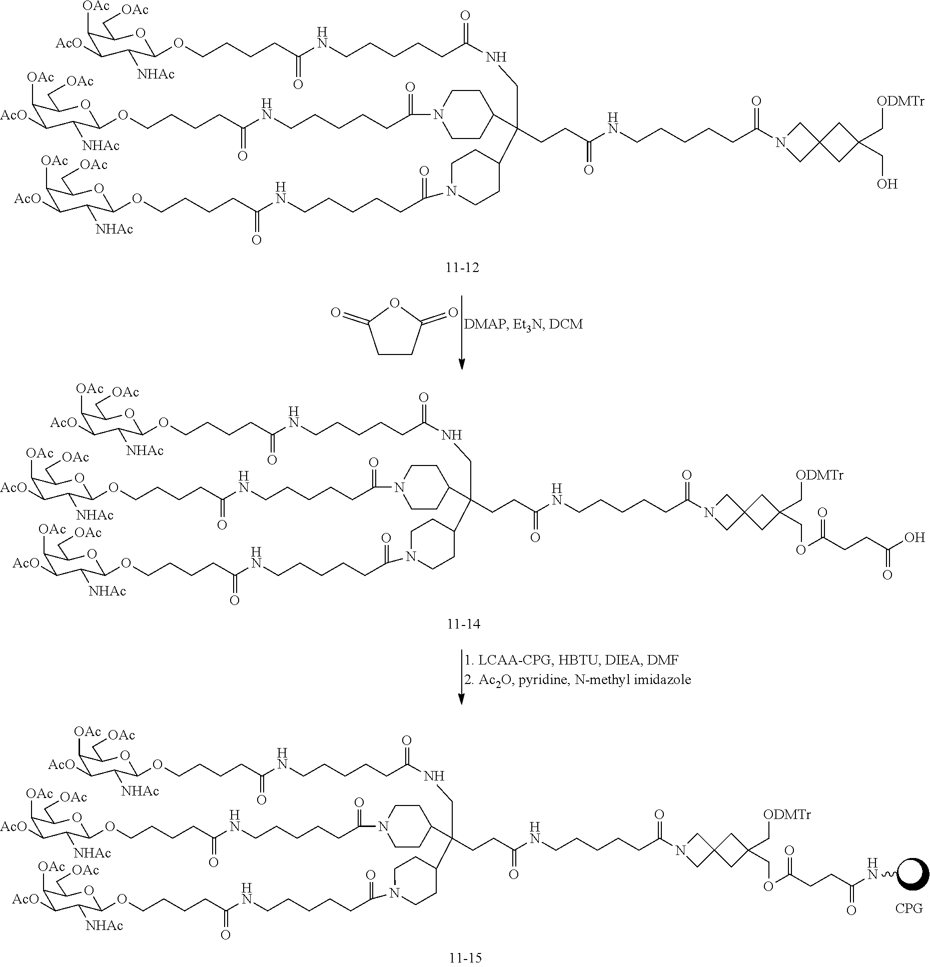

### Compound 11-14:

To a solution of compound 11-12 (150 mg, 0.06 mmol) in anhydrous DCM (1.0 mL), DMAP (5 mg, 0.04 mmol) and TEA (24 mg, 0.24 mmol) were added, followed by succinic anhydride (20 mg, 0.2 mmol) was added, the reaction mixture was stirred at rt for 3 h, LCMS showed the starting material was consumed completely, the reaction mixture was diluted with DCM and washed with water (3 mL×4), then washed with brine (3 mL×4), the organic layer was concentrated to give compound 11-14 (135 mg, 97% yield) as a white solid.

Mass calc. for C₁₂₉H₁₈₉N₁₁O₄₂: 2564.30; found: 1132.5 [M-DMTr+2H]²⁺, ESI.

¹H NMR (600 MHZ, DMSO-d₆): 7.84-7.71 (m, 6H), 7.45-7.21 (m, 10H), 6.92-6.89 (m, 4H), 5.21 (d, J=6.0 Hz, 3H), 4.98-4.94 (m, 3H), 4.49-4.44 (m, 5H), 4.07-3.37 (m, 36H), 3.16-2.84 (m, 14H), 2.44-1.77 (m, 62H), 1.45-1.21 (m, 50H).

### Compound 11-15:

To a solution of compound 11-14 (135 mg, 0.05 mmol) and HBTU (53 mg, 0.14 mmol) in anhydrous DMF (1.5 mL), DIPEA (30 mg, 0.23 mmol) was added. The reaction mixture was shaken at rt for 10 min, then native amino-lcaa-CPG (300 mg, loading 75 umol/g) was added, the suspension was shaken at rt for 20 h, then filtered, washed with DMF (20 mL×5), ACN (20 mL×5), DCM (20 mL×5) until TLC showed no spot at 254 nm in eluent. The solid support was dried under vacuum for 2 h to give solid support (300 mg). The unreacted amines on the support were capped by stirring with Ac₂O/pyridine/N-methyl imidazole (90 μL/1.0 mL/80 μL) at rt for 1 h, washed with DMF (20 mL×5), ACN (20 mL×5), DCM (20 mL×5) until TLC showed no spot at 254 nm in eluent. The solid support was dried under vacuum for 15 h to give solid support 11-15 (300 mg). For loading calculation, take 5.00 mg of the dried loaded CPG and add 20 mL of 3% DCA on DCM. Shake the solution and measure UV absorbance at 500 nm. Make sure the absorbance value is below 1.0 units to ensure no signal saturation. Then apply the following equation:

Loading in umol/g=(total volume of DCA added in mL)*(Abs value at 500 nm)*1000)/76*(mg of CPG taken)
Total volume of DCA added in mL=20 mL
Abs value at 500 nm=0.464
Mg of CPG taken=5.0 mg
Loading in umol/g=((20)*(0.464)*1000)/76*(5.0)=24 umol/g.

### Example 12

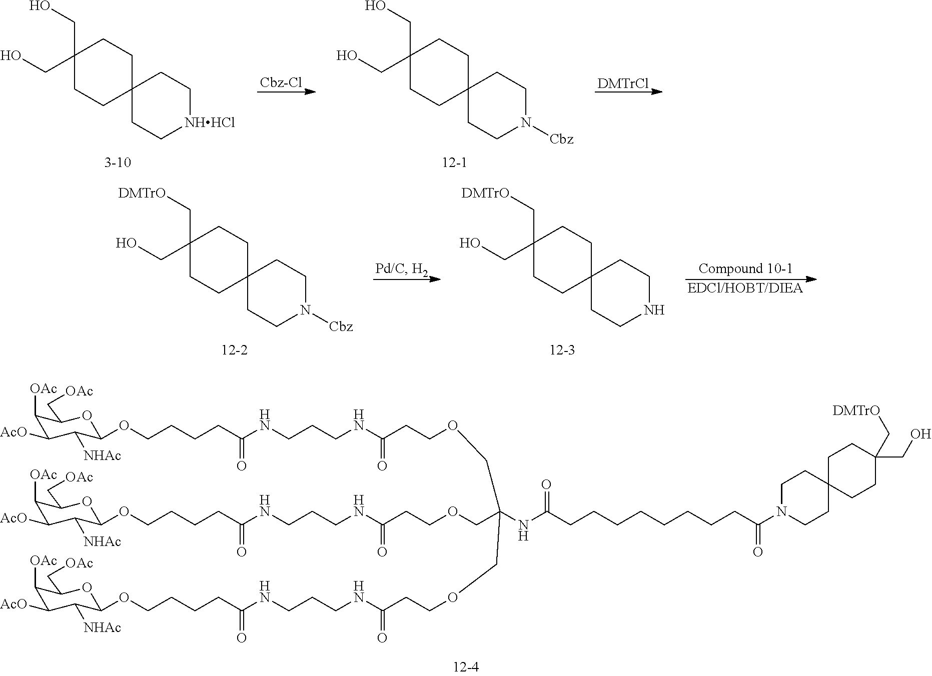

### Compound 12-1:

Compound 3-4 (1.1 g, 5.16 mmol) was dissolved in H₂O (10 mL) and THF (20 mL). K₂CO₃ (1.43 g, 10.31 mmol) and benzyl chloroformate (967 mg, 5.67 mmol) were added. The reaction mixture was stirred at rt for 3 h. Then it was concentrated under vacuum and purified by flash column chromatography (PE/EA=5/1) on silica gel to get compound 12-1 as a white foam solid (1.5 g, 84% yield).

Mass calc. for C₂₀H₂₉NO₄: 347.2; found: 348.3 [M+H]⁺, ESI.

### Compound 12-2:

Compound 12-1 (1.52 g, 4.37 mmol), DMAP (147.10 mg, 1.20 mmol) and DIEA (3.11 g, 24.08 mmol, 4.19 mL) were dissolved in DCM (20 mL). DMTrCl (1.48 g, 4.37 mmol) in DCM (20 mL) was slowly added to the reaction mixture at 0° C. The reaction was stirred at rt for 2 h. The reaction mixture was quenched with saturated NaHCO₃ solution (40 mL), extracted with DCM (50 mL×2), washed with brine (50 mL), dried over Na₂SO₄, concentrated under vacuum and purified by flash column chromatography (0-3% MeOH) on Al₂O₃ to give compound 12-4 as a white foam solid (1.9 g, 67% yield).

Mass calc. for C₄₁H₄₇NO₆: 649.34; found: 650.34 [M+H]⁺, ESI.

¹H NMR (400 MHZ, DMSO-d₆) δ 7.41-7.19 (m, 15H), 6.90-6.87 (m, 3H), 5.06-5.05 (m, 2H), 4.42-4.40 (m, 1H), 3.76 (s, 6H), 3.40-3.38 (m, 6H), 2.88 (s, 2H), 1.39-1.11 (m, 12H).

### Compound 12-3:

Compound 12-2 (0.7 g, 1.08 mmol) was dissolved in MeOH (30 mL), then Pd/C (140 mg) was added to the solution. The reaction mixture was stirred at rt for 3 h under hydrogen atmosphere. The reaction mixture was filtered and concentrated under vacuum to give crude compound 12-3 as a white foam solid (520 mg), which was used in the next step without further purification.

Mass calc. for C₃₃H₄₁NO₄: 515.30; found: 516.30 [M+H]⁺, ESI.

¹H NMR (400 MHZ, DMSO-d₆) δ 7.38 (d, J=8.8 Hz, 2H), 7.32-7.20 (m, 7H), 6.89-6.87 (m, 4H), 4.51-4.49 (m, 1H), 3.73 (s, 6H), 2.89-2.86 (m, 8H), 1.52-1.51 (m, 2H), 1.19-1.12 (m, 7H), 0.91-0.88 (m, 2H).

### Compound 12-4:

Compound 10-1 (1.56 g. 0.756 mmol) was dissolved in DCM (20 mL). DIEA (300.74 mg, 2.33 mmol), EDCI (297.39 mg, 1.55 mmol) and HOBT (209.62 mg, 1.55 mmol) were added to the mixture at 0° C. The reaction was stirred at 0° C. for 30 min. Compound 12-3 (400 mg, 0.756 mmol) was added to the reaction mixture. The reaction mixture was stirred at rt for 2 h. The reaction mixture was quenched with saturated NaHCO₃ solution (40 mL), extracted with DCM (30 mL×2), washed with brine (30 mL), dried over Na₂SO₄, concentrated under vacuum and purified by flash column chromatography (0-8% MeOH) on Al₂O₃ to give compound 12-4 as a white foam solid (1 g, 51% yield).

Mass calc. for C₁₂₄H₁₈₇N₁₁O₄₂: 2503.28; found: 1101.6 [M-DMTr+2H+]²⁺, ESI.

¹H NMR (400 MHZ, DMSO-d₆) δ 7.85-7.81 (m, 6H), 7.73 (t, J=5.6 Hz, 3H), 7.39 (d, J=7.32 Hz, 2H), 7.32-7.19 (m, 8H), 6.99 (s, 1H), 6.88 (d, J=8.9 Hz, 4H), 5.21-5.20 (m, 3H), 4.98-4.94 (m, 3H), 4.48 (d, J=8.44 Hz, 3H), 4.42 (t, J=5.0 Hz, 1H), 4.05-4.00 (m, 9H), 3.90-3.83 (m, 3H), 3.73-3.67 (m, 9H), 3.55-3.52 (m, 11H), 3.43-3.38 (m, 6H), 3.22-3.21 (m, 6H), 3.06-3.00 (m, 11H), 2.29-2.24 (m, 5H), 2.22 (t, J=7.4 Hz, 2H), 2.10 (s, 8H), 2.05 (t, J=6.96 Hz, 8H), 1.99-1.98 (m, 9H), 1.89 (s, 8H), 1.77 (s, 8H), 1.52-1.44 (m, 22H), 1.30-1.22 (m, 24H).

### Example 13

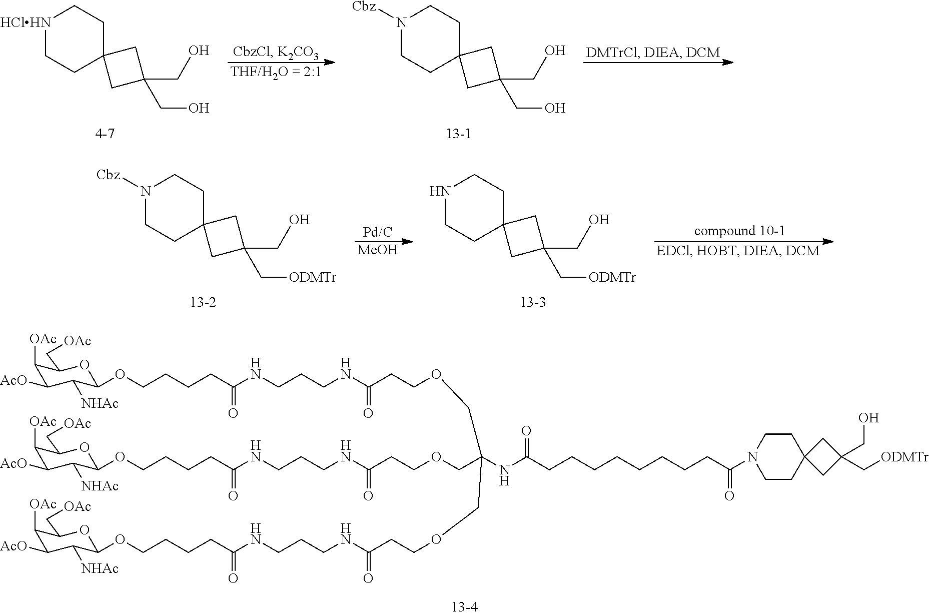

### Compound 13-1:

Compound 4-7 (1.3 g, 7.01 mmol) was dissolved in THF (30 mL) and H₂O (15 mL). K₂CO₃ (2.91 g, 21.05 mmol) was added to solution. And benzyl chloroformate (1.32 g, 7.72 mmol) was added to the reaction mixture at 0° C. The mixture was stirred at rt for 3 h. The reaction mixture was extracted with EA (50 mL×2), washed with brine (50 mL), dried over Na₂SO₄, concentrated under vacuum and purified by flash column chromatography (0-5% MeOH) on silica gel to get compound 13-1 as a white foam solid (1.95 g, 87% yield).

Mass calc. for C₁₈H₂₅NO₄: 319.2; found: 320.2 [M+H]⁺, ESI.

### Compound 13-2:

Compound 13-1 (2 g, 6.26 mmol) and DIEA (2.43 g, 18.79 mmol, 4.19 mL) were dissolved in DCM (60 mL). DMTrCl (2.02 g, 5.95 mmol) was added to the reaction mixture at 0° C. The reaction was stirred at 0° C. for 1 h. The reaction mixture was quenched with saturated NaHCO₃ solution (40 mL), extracted with DCM (50 mL×2), washed with brine (50 mL), dried over Na₂SO₄, concentrated under vacuum and purified by flash column chromatography (PE/EA=0-40%) on Al₂O₃ to give compound 13-2 as a white foam solid (2.6 g, 66% yield).

### Compound 13-3:

Compound 13-2 (600 mg, 0.96 mmol) was dissolved in MeOH (20 mL), then Pd/C was added to the reaction mixture. The reaction mixture was stirred at rt for 3 h under hydrogen atmosphere. The reaction mixture was filtered and concentrated under vacuum to give crude product 13-3 as a white foam solid (470 mg, crude), which was used in the next step without further purification.

Mass calc. for C₃₁H₃₇NO₄: 487.3; found: 488.3 [M+H]⁺, ESI.

Compound 13-4: Compound 10-1 (1.23 g, 0.61 mmol) was dissolved in DCM (20 mL). DIEA (239 mg, 1.85 mmol, 0.32 mL), EDCI (236 mg, 1.23 mmol) and HOBT (166 mg, 1.23 mmol) were added to the mixture at 0° C. The reaction was stirred at 0° C. for 30 min. Compound 13-3 (300 mg, 0.61 mmol) was added to the reaction mixture. The reaction mixture was stirred at rt for 2 h. The reaction mixture was quenched with saturated NaHCO₃ solution (40 mL), extracted with DCM (30 mL×2), washed with brine (30 mL), dried over Na₂SO₄, concentrated under vacuum and purified by flash column chromatography (0-8% MeOH) on Al₂O₃ to get compound 13-4 as a white foam solid (1 g, 66% yield).

¹H NMR (400 MHZ, DMSO-d₆) δ 7.85-7.82 (m, 6H), 7.75-7.73 (m, 3H), 7.40-7.38 (m, 2H), 733-7.29 (m, 2H), 7.27-7.25 (m, 4H), 7.23-7.19 (m, 1H), 6.98 (s, 1H), 6.90-6.88 (m, 4H), 5.21 (d, J=3.2 Hz, 3H), 4.97 (dd, J=3.2, 11.2 Hz, 3H), 4.63-4.61 (m, 1H), 4.48 (d, J=8.4 Hz, 3H), 4.04-4.00 (m, 9H), 3.91-3.83 (m, 3H), 3.73 (s, 6H), 3.71-3.67 (m, 3H), 3.55-3.52 (m, 12H), 3.44-3.37 (m, 5H), 3.27-3.15 (m, 5H), 3.06-3.01 (m, 14H), 2.27 (t, J=12.8 Hz, 6H), 2.21 (d, J=14.8 Hz, 2H), 2.10 (s, 9H), 2.04 (t, J=6.8 Hz, 8H), 1.99 (s, 9H), 1.89 (s, 9H), 1.77 (s, 9H), 1.58-1.33 (m, 30H), 1.29-1.23 (m, 12H).

### Example 14

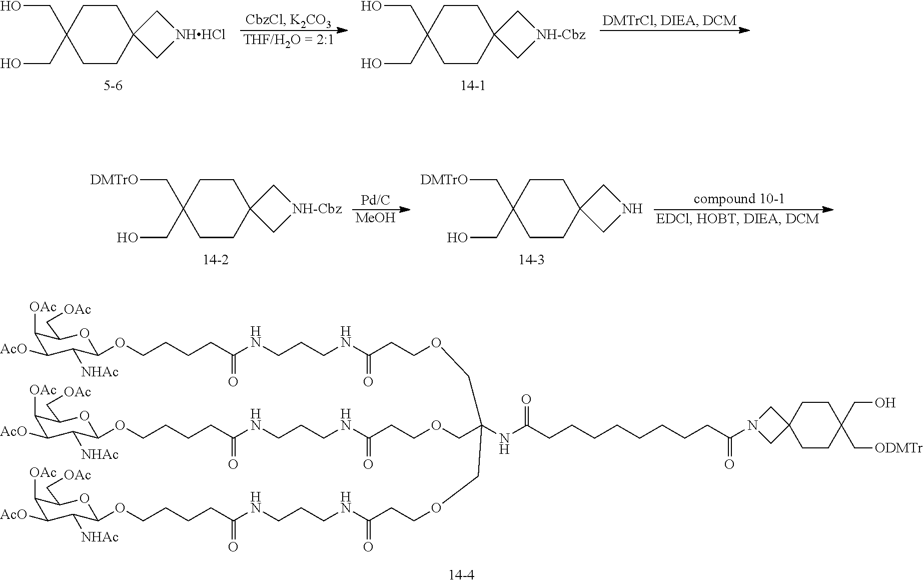

### Compound 14-1:

Compound 5-6 (3.6 g, 19.27 mmol) was dissolved in THF (30 mL) and H₂O (15 mL). K₂CO₃ (5.33 g, 38.54 mmol) was added to the solution. And benzyl chloroformate (3.62 g, 21.20 mmol) was added to the reaction mixture at 0° C. The mixture was stirred at rt for 2 h. The reaction mixture was extracted with EA (50 mL×2), washed with brine (50 mL), dried over Na₂SO₄, concentrated under vacuum and purified by flash column chromatography (0-2% MeOH) on silica gel to get compound 14-1 as white foam solid (5 g, 81% yield).

Mass calc. for C₁₈H₂₅NO₄: 319.2; found: 320.2 [M+H]⁺, ESI.

¹H NMR (400 MHZ, DMSO) δ 7.39-7.28 (m, 5H), 5.01 (s, 2H), 4.27 (t, J=5.4 Hz, 2H), 3.61-3.50 (m, 4H), 3.22 (d, J=5.3 Hz, 4H), 1.55 (t, J=6.1 Hz, 4H), 1.25-1.22 (m, 8H).

### Compound 14-2:

Compound 14-1 (3.5 g, 10.96 mmol) and DIEA (2.12 g, 16.44 mmol) were dissolved in DCM (60 mL). DMTrCl (3.34 g, 9.86 mmol) was added to the reaction mixture at 0° C. The reaction was stirred at 0° C. for 3 h. The reaction mixture was quenched with saturated NaHCO₃ solution (40 mL), extracted with DCM (50 mL×2), washed with brine (50 mL), dried over Na₂SO₄, concentrated under vacuum and purified by flash column chromatography (PE/EA=0-60%) on Al₂O₃ to get compound 14-2 as a white foam solid (5.5 g, 81% yield).

¹H NMR (400 MHz, DMSO-d₆) δ 7.39-7.18 (m, 14H), 6.88 (d, J=8.8 Hz, 4H), 5.00 (s, 2H), 4.39 (t, J=4.9 Hz, 1H), 3.73 (s, 6H), 3.58-3.35 (m, 6H), 2.83 (s, 2H), 1.29-1.15 (m, 8H).

### Compound 14-3:

Compound 14-2 (254 mg, 408.52 μmol) was dissolved in MeOH (10 mL), then Pd/C was added to the reaction mixture. The reaction mixture was stirred at rt for 2 h under hydrogen atmosphere. The reaction mixture was filtered and concentrated under vacuum to give crude product 14-3 as a white foam solid (199 mg, crude), which was used in the next step without further purification.

Mass calc. for C₃₁H₃₇NO₄: 487.3; found: 488.3 [M+H]⁺, ESI.

### Compound 14-4:

Compound 10-1 (820 mg, 408.51 μmol) was dissolved in DCM (20 mL). DIEA (158 mg, 1.23 mmol), EDCI (157 mg, 817 μmol) and HOBT (110 mg, 817 μmol) were added to the mixture at 0° C. The reaction was stirred at 0° C. for 30 min. Compound 14-3 (199 mg, 408.51 μmol) was added to the reaction mixture. The reaction mixture was stirred at rt for 2 h. The reaction mixture was quenched with saturated NaHCO₃ solution (40 mL), extracted with DCM (30 mL×2), washed with brine (30 mL), dried over Na₂SO₄, concentrated under vacuum and purified by flash column chromatography (0-6% MeOH) on Al₂O₃ to get compound 14-4 as a white foam solid (540 mg, 53% yield).

¹H NMR (400 MHZ, DMSO-d₆) δ 7.89-7.80 (m, 6H), 7.75 (t, J=5.5 Hz, 3H), 7.37 (d, J=7.3 Hz, 2H), 7.30 (t, J=7.7 Hz, 2H), 7.23-7.17 (m, 5H), 6.99 (s, 1H), 6.87 (d, J=8.4 Hz, 4H), 5.21 (d, J=3.4 Hz, 3H), 4.96 (dd, J=10.8, 3.4 Hz, 3H), 4.47 (d, J=8.5 Hz, 3H), 4.41 (t, J=4.9 Hz, 1H), 4.04-3.98 (m, 9H), 3.91-3.82 (m, 3H), 3.74-3.48 (m, 23H), 3.43-3.36 (m, 6H), 3.06-2.98 (m, 12H), 2.83 (d, J=9.4 Hz, 2H), 2.27 (t, J=6.3 Hz, 6H), 2.09 (s, 9H), 2.03 (t, J=7.0 Hz, 8H), 2.00-1.95 (m, 12H), 2.01 (s, 9H), 1.89 (s, 9H), 1.77 (s, 9H), 1.54-1.37 (m, 22H), 1.31-1.14 (m, 20H).

### Example 15

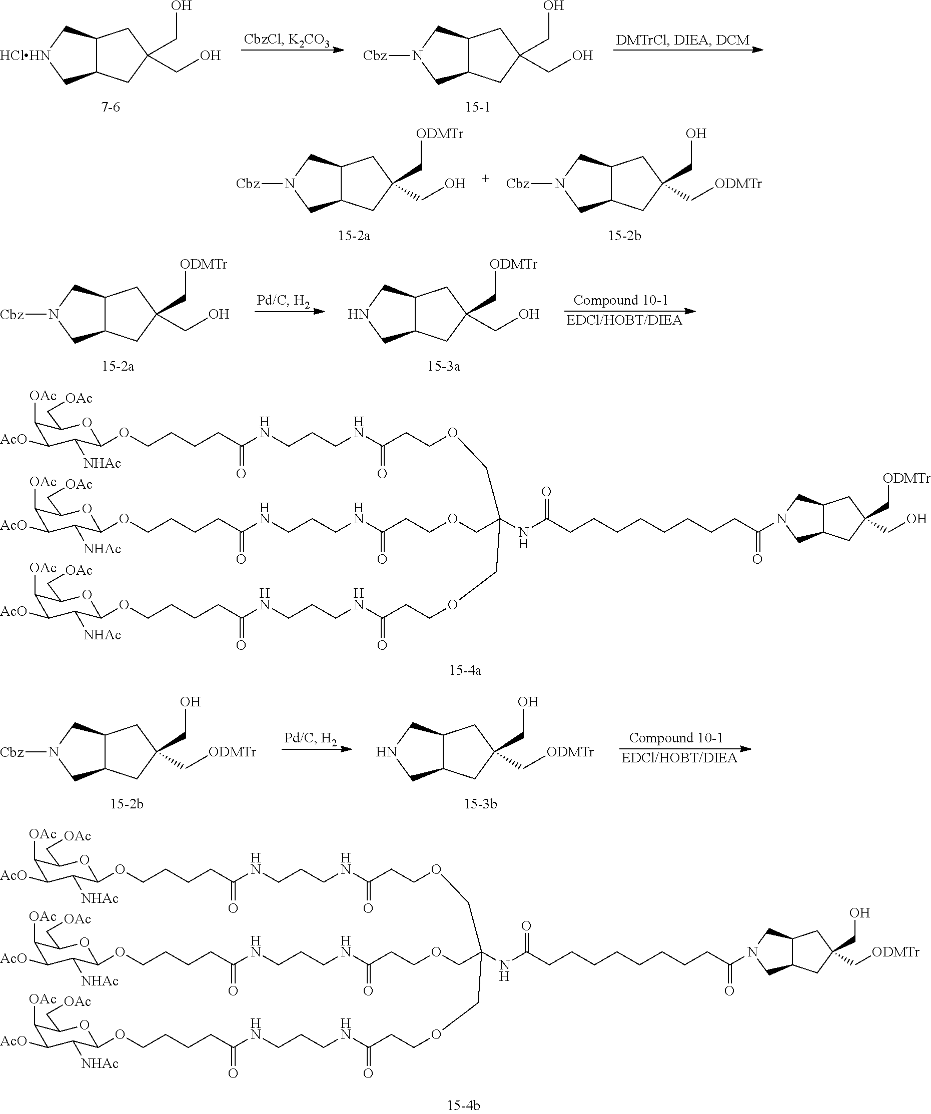

### Compound 15-1:

Compound 7-6 (3.8 g, 18.30 mmol) was dissolved in THF (30 mL) and H₂O (15 mL). K₂CO₃ (5.06 g, 36.59 mmol) was added to the reaction mixture. And benzyl chloroformate (3.43 g, 20.13 mmol) was added to the reaction mixture at 0° C. The mixture was stirred at rt for 2 h. The reaction mixture was extracted with EA (50 mL×2), washed with brine (50 mL), dried over Na₂SO₄, concentrated under vacuum to give the crude product 15-1 as a white foam (3.46 g, crude).

Mass calc. for C₁₇H₂₃NO₄: 305.2; found: 306.2 [M+H]⁺, ESI.

### Compound 15-2:

Compound 15-1 (3.07 g, 10.06 mmol) and DIEA (1.95 g, 15.09 mmol) were dissolved in DCM (60 mL). DMTrCl (3.41 g, 10.06 mmol) was added to the reaction mixture at 0° C. The reaction was stirred at 0° C. for 3 h. The reaction mixture was quenched with saturated NaHCO₃ solution (40 mL), extracted with DCM (50 mL×2), washed with brine (50 mL), dried over Na₂SO₄, concentrated under vacuum and purified by flash column chromatography (PE/EA=0-60%) on Al₂O₃ and prep-HPLC to give compound 15-2a as a white foam solid (1.04 g, 34% yield) and compound 15-2b (1.78 g, 58% yield).

15-2a:

¹H NMR (400 MHZ, DMSO-d₆) δ 7.38-7.36 (m, 1H), 7.36-7.31 (m, 5H), 7.31-7.20 (m, 7H), 7.20-7.15 (m, 1H), 6.85 (d, J=8.9 Hz, 4H), 5.02 (d, J=6.1 Hz, 2H), 6.98 (s, 1H), 4.61 (t, J=5.0 Hz, 1H), 3.71 (s, 6H), 3.46-3.35 (m, 2H), 3.27 (d, J=5.0 Hz, 2H), 3.13-3.05 (m, 2H), 2.93 (s, 2H), 2.64-2.55 (m, 2H), 1.72-1.62 (m, 2H), 1.30-1.21 (m, 2H).

15-2b:

¹H NMR (400 MHZ, DMSO-d₆) δ 7.40-7.27 (m, 9H), 7.27-7.22 (m, 4H), 7.22-7.16 (m, 1H), 6.89 (d, J=8.9 Hz, 4H), 5.03 (s, 2H), 6.98 (s, 1H), 4.64 (t, J=5.1 Hz, 1H), 3.73 (s, 6H), 3.43-3.34 (m, 4H), 3.19-3.09 (m, 2H), 3.13-3.05 (m, 2H), 2.84 (s, 2H), 2.41-2.31 (m, 2H), 1.62-1.54 (m, 2H), 1.38-1.30 (m, 2H).

### Compound 15-3:

Compound 15-2a (210 mg, 350.48 μmol) was dissolved in MeOH (10 mL), then Pd/C was added to the solution. The reaction mixture was stirred at rt for 2 h under hydrogen atmosphere. The reaction mixture was filtered and concentrated under vacuum to give crude product 15-3a as a white foam solid (166 mg, crude), which was used in the next step without further purification.

Mass calc. for C₃₀H₃₅NO₄: 473.3; found: 474.3 [M+H]⁺, ESI.

Compound 15-2b (610 mg, 1.00 mmol) was dissolved in MeOH (10 mL), then Pd/C was added to the solution. The reaction mixture was stirred at rt for 2 h under hydrogen atmosphere. The reaction mixture was filtered and concentrated under vacuum to give crude product 15-3b as a white foam solid (475 mg, crude), which was used in the next step without further purification.

### Compound 15-4:

Compound 10-1 (700 mg, 348.92 μmol) was dissolved in DCM (20 mL). DIEA (135 mg, 1.05 mmol), EDCI (134 mg, 698 μmol) and HOBT (94 mg, 698 μmol) were added to the mixture at 0° C. The reaction was stirred at 0° C. for 30 min. Compound 15-3a (199 mg, 408.51 μmol) was added to the reaction mixture. The reaction mixture was stirred at rt for 2 h. The reaction mixture was quenched with saturated NaHCO₃ solution (40 mL), extracted with DCM (30 mL×2), washed with brine (30 mL), dried over Na₂SO₄, concentrated under vacuum and purified by flash column chromatography (0-6% MeOH) on Al₂O₃ to get compound 15-4a as a white foam solid (550 mg, 64% yield).

¹H NMR (400 MHZ, DMSO-d₆) δ 7.88-7.80 (m, 6H), 7.75 (t, J=5.4 Hz, 3H), 7.38-7.34 (m, 2H), 7.28 (t, J=7.6 Hz, 2H), 7.24-7.17 (m, 5H), 7.00 (s, 1H), 6.87 (d, J=8.9 Hz, 4H), 5.21 (d, J=3.4 Hz, 3H), 4.96 (dd, J=11.3, 3.4 Hz, 3H), 4.64-4.60 (m, 1H), 4.47 (d, J=8.5 Hz, 3H), 4.05-3.98 (m, 9H), 3.92-3.83 (m, 3H), 3.76-3.65 (m, 9H), 3.62-3.45 (m, 13H), 3.45-3.37 (m, 6H), 3.28-3.23 (m, 2H), 3.17-3.09 (m, 2H), 3.07-2.98 (m, 12H), 2.97-2.89 (m, 2H), 2.69-2.53 (m, 2H), 2.27 (t, J=6.2 Hz, 6H), 2.16-2.07 (m, 11H), 2.06-1.96 (m, 17H), 1.88 (s, 9H), 1.78 (s, 9H), 1.57-1.36 (m, 22H), 1.26-1.15 (m, 14H).

Compound 10-1 (1 g, 500.29 μmol) was dissolved in DCM (20 mL). DIEA (194 mg, 1.50 mmol), EDCI (192 mg, 1.00 mmol) and HOBT (135 mg, 1.00 mmol) were added to the mixture at 0° C. The reaction was stirred at 0° C. for 30 min. Compound 15-3b (238 mg, 502.53 μmol) was added to the reaction mixture. The reaction mixture was stirred at rt for 2 h. The reaction mixture was quenched with saturated NaHCO₃ solution (40 mL), extracted with DCM (30 mL×2), washed with brine (30 mL), dried over Na₂SO₄, concentrated under vacuum and purified by flash column chromatography (0-6% MeOH) on Al₂O₃ to get compound 15-4b as a white foam solid (550 mg, 44% yield).

¹H NMR (400 MHZ, DMSO-d₆) δ 7.87-7.80 (m, 4H), 7.74 (t, J=5.6 Hz, 3H), 7.40-7.36 (m, 2H), 7.31 (t, J=7.7 Hz, 2H), 7.27-7.18 (m, 5H), 6.99 (s, 1H), 6.91-6.87 (m, 4H), 5.22 (d, J=3.4 Hz, 3H), 4.96 (dd, J=11.3, 3.4 Hz, 3H), 4.66-4.62 (m, 1H), 4.48 (d, J=8.4 Hz, 3H), 4.05-3.98 (m, 9H), 3.92-3.83 (m, 3H), 3.75-3.67 (m, 9H), 3.57-3.47 (m, 11H), 3.46-3.36 (m, 6H), 3.34-3.27 (m, 4H), 3.23-3.09 (m, 2H), 3.07-2.98 (m, 12H), 2.85 (s, 2H), 2.46-2.31 (m, 2H), 2.30-2.24 (m, 6H), 2.18-2.12 (m, 2H), 2.09 (s, 9H), 2.06-2.01 (m, 8H), 1.99 (s, 9H), 1.88 (s, 9H), 1.77 (s, 9H), 1.55-1.37 (m, 22H), 1.33-1.14 (m, 14H).

### Example 16

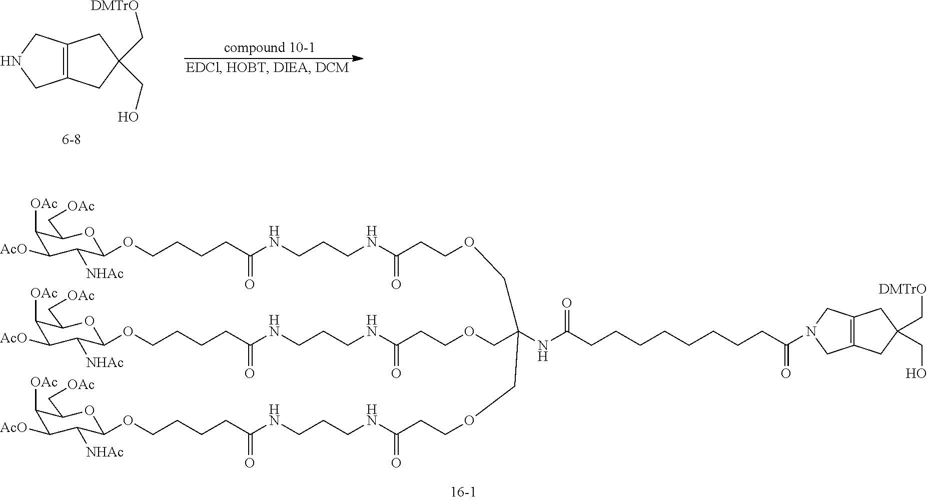

### Compound 16-1:

Compound 6-8 (190 mg, 0.403 mmol) and compound 10-1 (809 mg, 0.403 mmol) were dissolved in DCM (10 mL), then DIEA (156.35 mg, 1.21 mmol), EDCI (154.61 mg, 0.81 mmol) and HOBT (108.97 mg, 0.81 mmol) were added to the mixture. The reaction was stirred at rt for 2 h. The reaction mixture was quenched with saturated NaHCO₃ solution (40 mL), extracted with DCM (30 mL×2), washed with brine (30 mL), dried over Na₂SO₄, concentrated under vacuum and purified by flash column chromatography (0-10% MeOH) on Al₂O₃ to obtain compound 16-1 as a white foam solid (772 mg, 77% yield).

Mass calc. for C₁₂₁H₁₇₉N₁₁O₄₂: 2458.22; found: 1079.2 [M-DMTr+2H]²⁺, ESI.

¹H NMR (400 MHZ, DMSO-d₆) δ 7.85-7.82 (m, 6H), 7.75-7.72 (m, 3H), 7.38-7.36 (m, 2H), 7.29-7.27 (m, 2H), 7.25-7.22 (m, 5H), 6.98 (s, 1H), 6.87 (d, J=8.0 Hz, 4H), 5.21 (d, J=4.0 Hz, 3H), 4.97 (dd, J=3.2, 8.0 Hz, 3H), 4.80-4.70 (m, 1H), 4.48 (d, J=8.4 Hz, 3H), 4.02-4.01 (m, 11H), 3.88-3.86 (m, 5H), 3.73-3.69 (m, 9H), 3.55-3.52 (m, 12H), 3.46-3.36 (m, 5H), 3.17 (d, J=5.2 Hz, 1H), 3.04-3.00 (m, 14H), 2.29-2.26 (m, 10H), 2.10-2.07 (m, 8H), 2.06-2.02 (m, 19H), 1.99 (s, 9H), 1.77 (s, 9H), 1.52-1.46 (m, 22H), 1.45-1.22 (m, 12H).

### Example 17

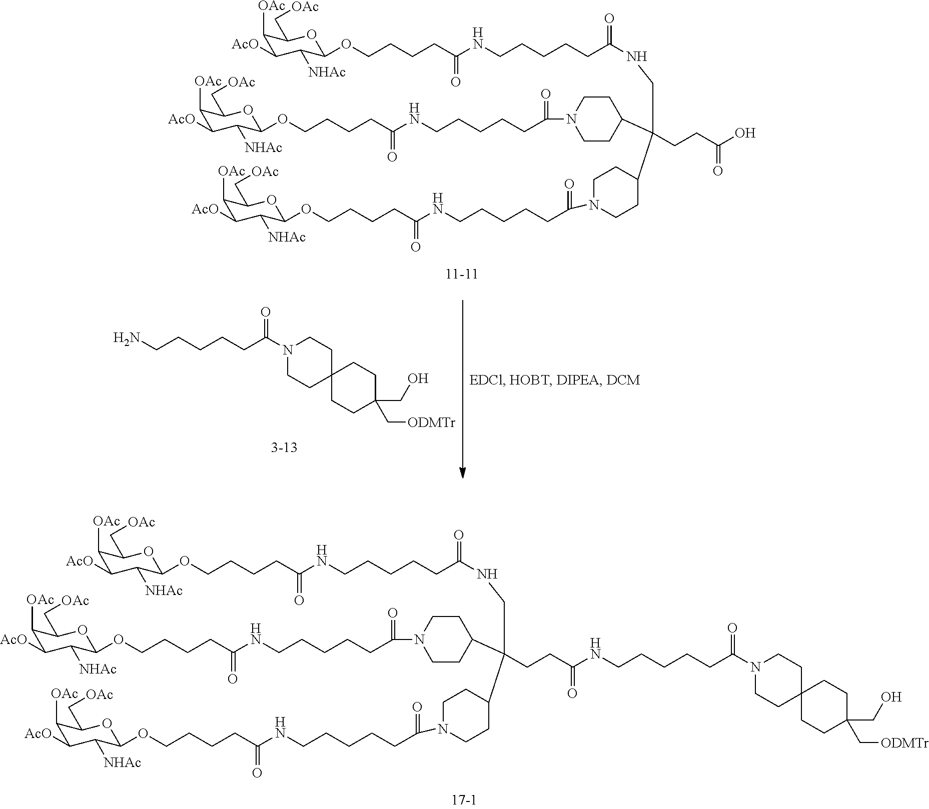

Compound 17-1: Compound 11-11 (1.5 g, 0.78 mmol), DIEA (302.32 mg, 2.35 mmol), HOBT (212.11 mg, 1.57 mmol) and EDCI (300.92 mg, 1.57 mmol) was dissolved in DCM (15 mL) at 0° C., then compound 3-12 (493.56 mg, 0.78 mmol) was added, and the mixture was stirred at rt for 1 h. LC-MS showed full conversion. The reaction mixture was diluted by adding DCM (20 mL), and washed by NaHCO₃ (sat. Aq., 50 mL), and water (50 mL), concentrated and purified by prep-HPLC (C18 column, ACN/water) to afford compound 17-1 (1.14 g, 58% yield).

Mass calc for C₁₂₉H₁₉₃N₁₁O₃₉ 2520.35, found 1109.35 (M-DMTr+2H)²⁺, ESI.

¹H NMR (400 MHZ, DMSO-d₆) δ 7.83 (d, J=9.2 Hz, 4H), 7.73 (t, J=9.2 Hz, 3H), 7.47-7.39 (m, 3H), 7.33-7.19 (m, 7H), 6.88 (d, J=8.92 Hz, 4H), 5.21 (d, J=3.3 Hz, 3H), 4.98-4.94 (m, 3H), 4.49-4.42 (m, 5H), 4.02 (s, 9H), 3.91-3.86 (m, 5H), 3.73-3.68 (m, 9H), 3.43-3.39 (m, 5H), 3.34-3.30 (m, 3H), 3.02-2.99 (m, 10H), 2.87 (s, 4H), 2.34-2.33 (m, 2H), 2.25-2.21 (m, 6H), 2.10 (s, 9H), 2.10-2.08 (m, 8H), 2.0 (s, 9H), 1.99 (s, 3H), 1.89 (s, 9H), 1.77 (s, 9H), 1.45-1.24 (m, 64H).

### Example 18

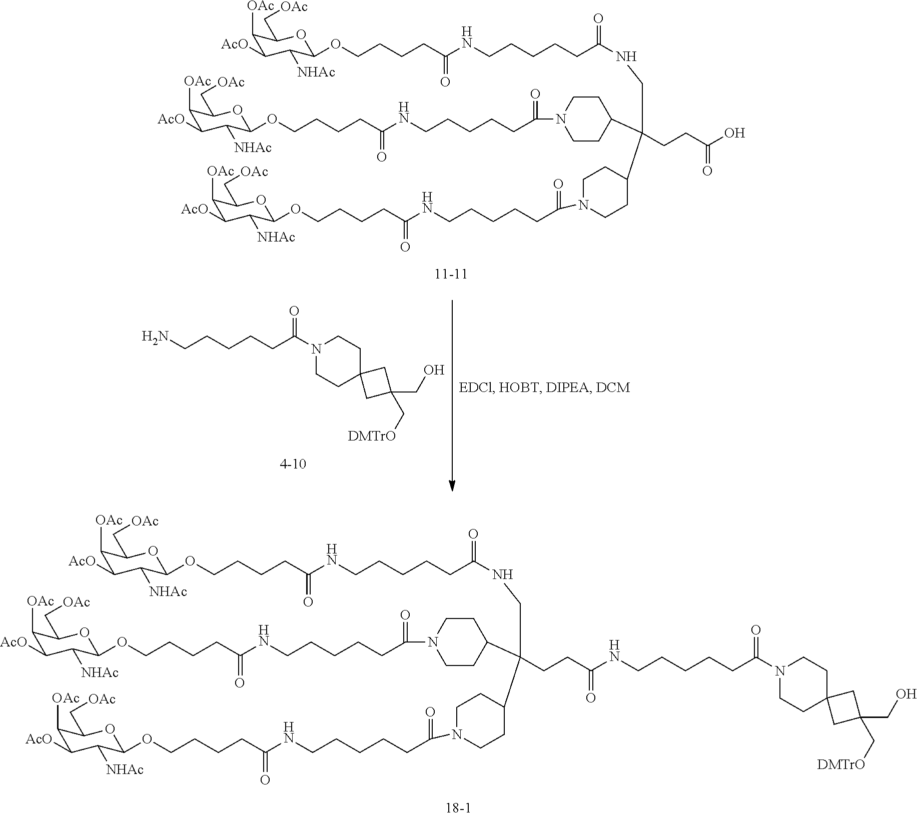

### Compound 18-1:

To a solution of compound 11-11 (1.01 g, 0.45 mmol) in DCM (20 mL) were added DIEA (193.61 mg, 1.85 mmol, 0.32 mL), EDCI (191.45 mg, 0.99 mmol) and HOBT (134.95 mg, 0.99 mmol) at 0° C. The reaction was stirred at 0° C. for 30 min. Compound 4-10 (300 mg, 0.49 mmol) was added to the reaction mixture. The reaction mixture was stirred at rt for 2 h. The reaction mixture was quenched with saturated NaHCO₃ solution (40 mL), extracted with DCM (30 mL×2), washed with brine (30 mL), dried over Na₂SO₄, concentrated under vacuum and purified by flash column chromatography (0-8% MeOH) on Al₂O₃ to give compound 18-1 as a white foam solid (500 mg, 46% yield).

Mass calc. for C₁₂₇H₁₈₉N₁₁O₃₉: 2493.31; found: 1096.7 [M-DMTr+2H]²⁺, ESI.

¹H NMR (400 MHZ, DMSO-d₆) δ 7.84-7.78 (m, 2H), 7.72 (t, J=5.4 Hz, 3H), 7.45 (br, 1H), 7.40-7.39 (m, 2H), 7.33-7.30 (m, 2H), 7.28-7.20 (m, 5H), 5.22 (d, J=3.4 Hz, 3H), 4.99-4.95 (m, 3H), 4.64-4.62 (m, 3H), 4.49 (d, J=8.4 Hz, 2H), 4.03 (s, 9H), 3.91-3.84 (m, 5H), 3.74 (s, 6H), 3.72-3.68 (m, 3H), 3.45-3.41 (m, 2H), 3.40-3.34 (m, 4H), 3.28-3.16 (m, 4H), 3.01-2.99 (m, 11H), 2.85-2.67 (m, 4H), 2.34-2.32 (m, 2H), 2.27-2.22 (m, 6H), 2.07-2.00 (m, 17H), 1.89 (s, 9H), 1.78 (s, 9H), 1.64-1.56 (m, 4H), 1.55-1.34 (m, 36H), 1.28-1.21 (m, 16H).

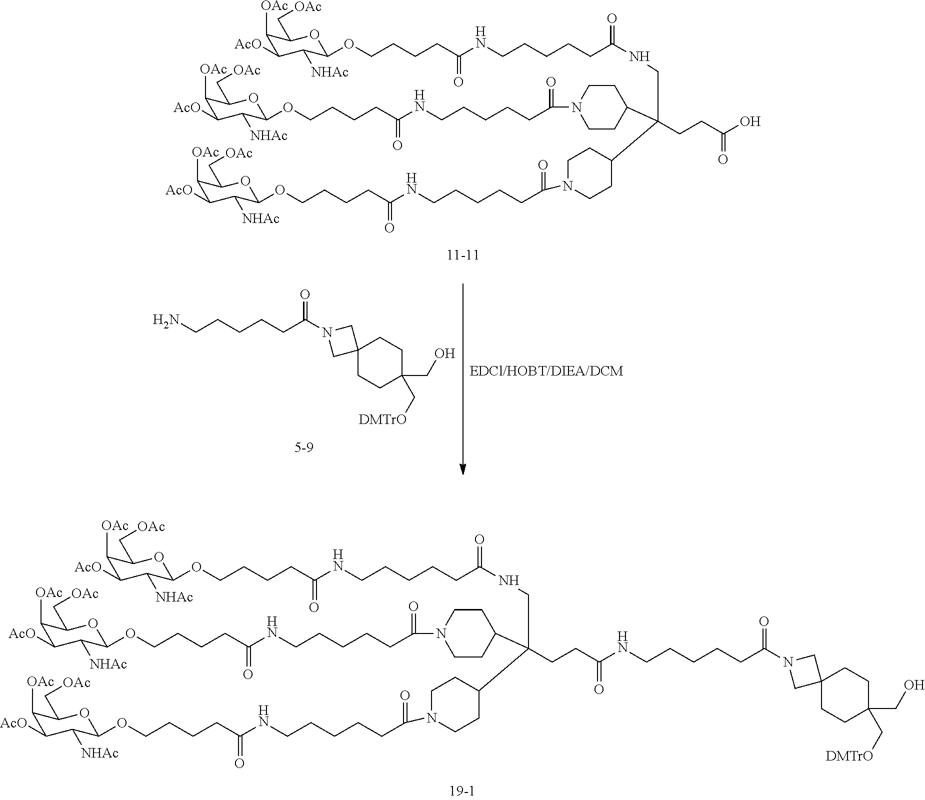

### Example 19

### Compound 19-1:

Compound 11-11 (1.59 g, 832.24 μmol) (3.2 g, 7.16 mmol) and compound 5-9 (500 mg, 832.24 μmol) were dissolved in DCM (50 mL). DIEA (215.12 mg, 1.66 mmol, 289.92 μL), EDCI (239.31 mg, 1.25 mmol) and HOBT (112.45 mg, 832.24 μmol) were added to the mixture. The reaction was stirred at rt overnight. The reaction mixture was quenched with water (40 mL), extracted with DCM (50 mL×2), washed with brine (50 mL), dried over Na₂SO₄, concentrated under vacuum and purified by HPLC to give compound 19-1 (840 mg, 36% yield).

Mass calc. for C₁₂₇H₁₈₉N₁₁O₃₉: 2492.3; found: 1258.4 [M+2Na]²⁺, ESI.

¹H NMR (400 MHZ, DMSO-d₆) δ 7.85-7.71 (m, 7H), 7.49 (s, 1H), 7.37-7.20 (m, 9H), 6.82-6.87 (m, J=8.8 Hz, 4H), 5.22 (d, J=3.2 Hz, 3H), 4.97 (d, J=11.6 Hz, 3H), 4.49-4.46 (m, 6H), 4.05 (s, 9H), 3.91-3.85 (m, 5H), 3.77-3.62 (m, 11H), 3.41 (s, 6H), 3.01-2.71 (m, 14H), 2.32-2.25 (m, 6H), 2.11-1.78 (m, 48H), 1.62-1.34 (m, 37H), 1.26-1.21 (m, 20H).

### Example 20

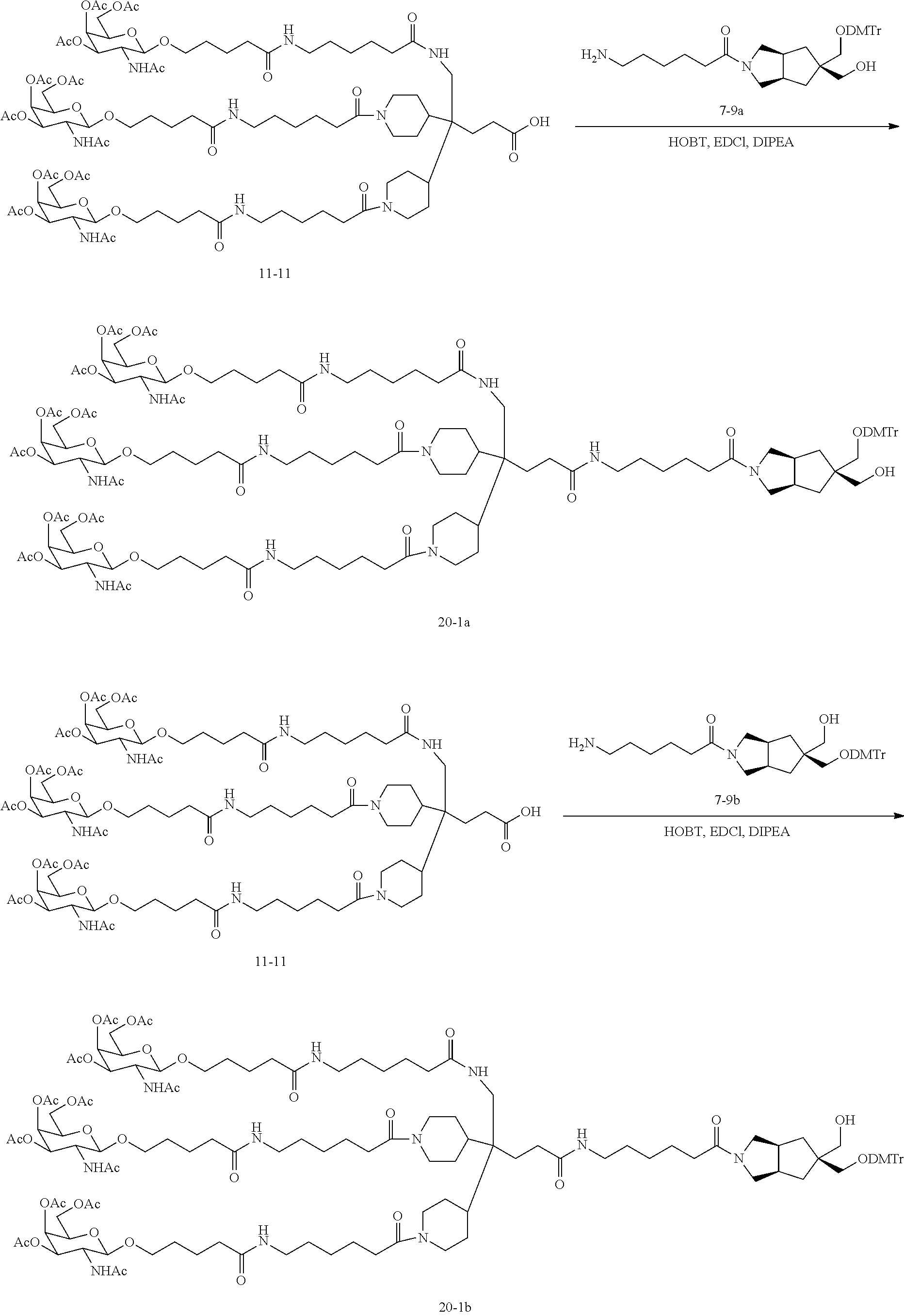

### Compound 20-1a:

Compound 11-11 (554 mg, 289.73 μmol) was dissolved in DCM (20 mL). DIEA (112 mg, 869.18 μmol), EDCI (222 mg, 1.16 mmol) and HOBT (157 mg, 1.16 mmol) were added to the mixture at 0° C. The reaction was stirred at 0° C. for 30 min. Compound 7-9a (170 mg, 289.73 μmol) was added to the reaction mixture and the mixture was stirred at rt for 2 h. The reaction was quenched with saturated NaHCO₃ solution (40 mL), extracted with DCM (30 mL×2), washed with brine (30 mL), dried over Na₂SO₄, concentrated and purified by prep-HPLC (C18 column, ACN/water) to afford compound 20-1a as a white foam solid (640 mg, 89% yield).

¹H NMR (400 MHZ, DMSO-d₆) δ 7.86-7.76 (m, 4H), 7.72 (t, J=5.4 Hz, 3H), 7.49-7.42 (m, 1H), 7.38 (d, J=8.6 Hz, 2H), 7.31 (t, J=7.7 Hz, 2H), 7.27-7.19 (m, 5H), 6.88 (d, J=8.9 Hz, 4H), 5.21 (d, J=3.4 Hz, 3H), 4.96 (dd, J=11.2, 3.4 Hz, 3H), 4.65 (t, J=5.0 Hz, 1H), 4.50-4.41 (m, 5H), 4.05-3.99 (m, 9H), 3.91-3.82 (m, 5H), 3.76-3.66 (m, 9H), 3.49-3.36 (m, 6H), 3.32-3.28 (m, 1H), 3.23-3.09 (m, 2H), 3.08-2.91 (m, 10H), 2.84 (s, 2H), 2.44-2.21 (m, 8H), 2.20-1.92 (m, 30H), 1.88 (s, 9H), 1.77 (s, 9H), 1.70-1.08 (m, 54H).

### Compound 20-1b:

Compound 11-11 (1.3 g, 681.71 μmol) was dissolved in DCM (20 mL). DIEA (176 mg, 1.36 mmol), EDCI (261 mg, 1.36 mmol) and HOBT (138 mg, 1.02 mmol) was added to the mixture at 0° C. The reaction was stirred at 0° C. for 30 min. Compound 7-9b (400 mg, 681.71 μmol) was added to the reaction mixture. The reaction mixture was stirred at rt for 2 h.

The reaction mixture was quenched with saturated NaHCO₃ solution (40 mL), extracted with DCM (30 mL×2), washed with brine (30 mL), dried over Na₂SO₄, concentrated and purified by prep-HPLC (C18 column, ACN/water) to afford compound 20-1b as a white foam solid (610 mg, 36% yield).

¹H NMR (400 MHZ, DMSO-d₆) δ: 7.87-7.76 (m, 4H), 7.72 (t, J=5.3 Hz, 3H), 7.46 (s, 1H), 7.35 (d, J=7.4 Hz, 2H), 7.28 (t, J=7.6 Hz, 2H), 7.25-7.18 (m, 5H), 6.86 (d, J=8.9 Hz, 4H), 5.21 (d, J=3.4 Hz, 3H), 4.96 (dd, J=11.2, 3.4 Hz, 3H), 4.62 (t, J=5.0 Hz, 1H), 4.50-4.38 (m, 5H), 4.07-3.98 (m, 9H), 3.92-3.82 (m, 5H), 3.75-3.66 (m, 9H), 3.51-3.36 (m, 6H), 3.29-3.23 (m, 2H), 3.15-3.07 (m, 2H), 3.01-2.94 (m, 10H), 2.93-2.88 (m, 2H), 2.87-2.78 (m, 2H), 2.68-2.60 (m, 1H), 2.37-2.23 (m, 2H), 2.29-2.19 (m, 4H), 2.13-2.08 (m, 10H), 2.07-1.98 (m, 18H), 1.88 (s, 9H), 1.77 (s, 9H), 1.71-1.58 (m, 6H), 1.53-1.13 (m, 48H).

### Example 21

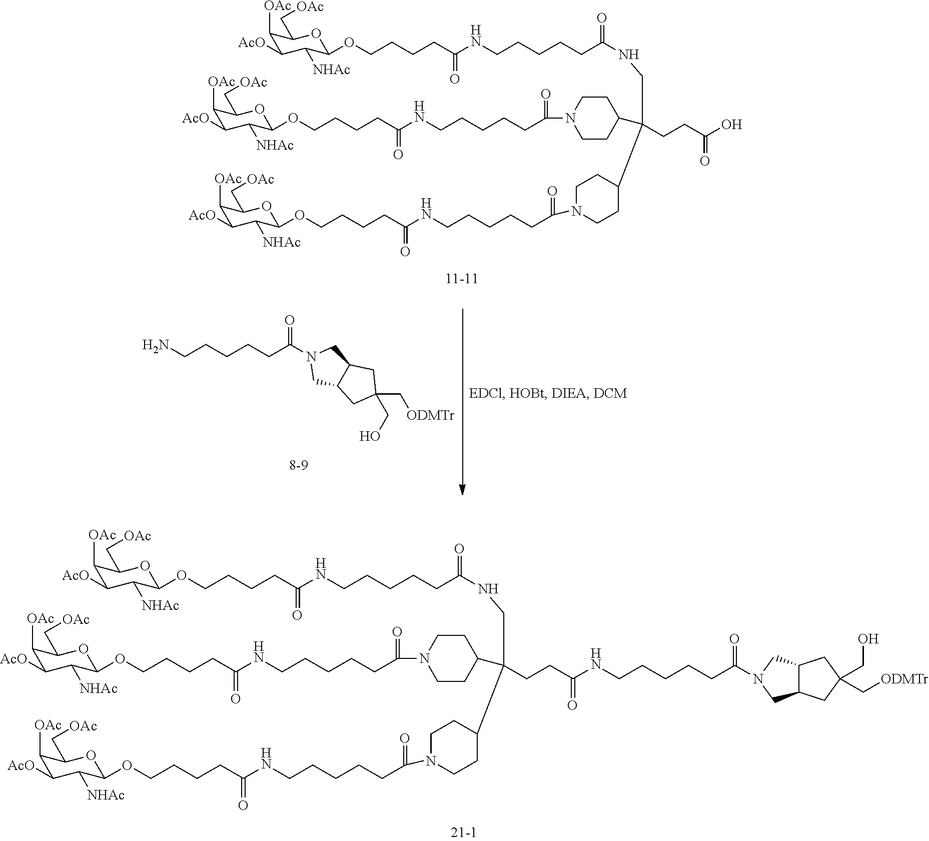

### Compound 21-1:

Compound 11-11 (1.76 g, 920.31 μmol), HOBt (248.70 mg, 1.84 mmol), EDCI (529.27 mg, 2.76 mmol) were dissolved in DCM (20 mL). The mixture was stirred for 30 min, then compound 8-9 (540 mg, 920.31 μmol) was added. The reaction mixture was stirred for another 2 hours, then it was concentrated and purified by prep-HPLC to give compound 21-1 (1.2 g, 53% yield).

Mass calc. for C₁₂₆H₁₈₇N₁₁O₃₉: 2478.3; found: 1089.6 [M-DMTr+2H]²⁺, ESI.

¹H NMR (400 MHZ, DMSO-d₆) δ 7.84-7.80 (m, 4H), 7.72 (t, J=5.4 Hz, 3H), 7.39 (s, 1H), 7.40-7.37 (m, 2H), 7.31 (t, J=7.7 Hz, 2H), 7.27-7.20 (m, 5H), 6.98 (d, J=8.9 Hz, 4H), 5.22 (d, J=3.4 Hz, 3H), 4.97 (dd, J=3.4, 7.8 Hz, 3H), 4.67 (t, J=4.8 Hz, 1H), 4.48 (d, J=8.5 Hz, 3H), 4.47-4.42 (m, 2H), 4.05-3.98 (m, 9H), 3.92-3.83 (m, 5H), 3.73 (s, 6H), 3.72-3.67 (m, 3H), 3.55-3.45 (m, 2H), 3.44-3.35 (m, 5H), 3.05-2.93 (m, 11H), 2.91-2.80 (m, 3H), 2.65-2.55 (m, 2H), 2.38-2.20 (m, 9H), 2.10 (s, 9H), 2.08-2.01 (m, 9H), 2.00 (s, 9H), 1.89 (s, 9H), 1.78 (s, 9H), 1.69-1.52 (m, 6H), 1.47-0.98 (m, 48H).

### Example 22

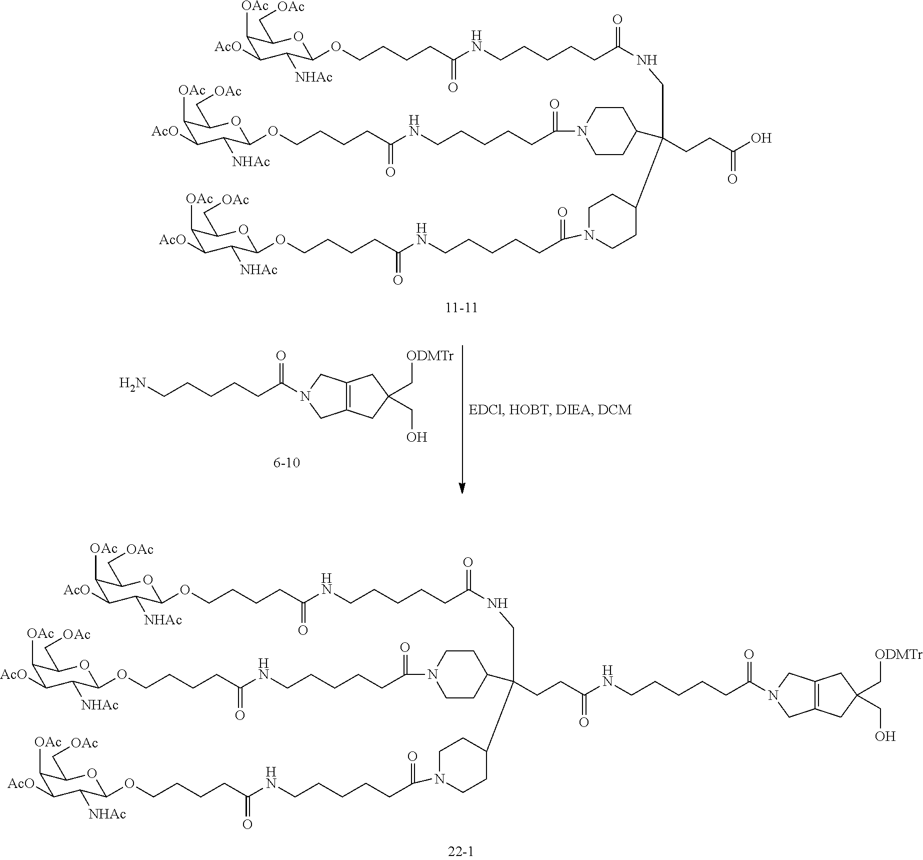

### Compound 22-1:

Compound 6-10 (410 mg. 0.71 mmol) and compound 11-11 (1.34 g, 0.71 mmol) were dissolved in DCM (60 mL), then DIEA (453.09 mg, 3.51 mmol), EDCI (299.57 mg, 1.75 mmol) and HOBT (236.85 mg, 1.75 mmol) were added to the solution. The reaction mixture was stirred at rt for 2 h. The reaction was quenched with saturated NaHCO₃ solution (100 mL), extracted with DCM (60 mL×3), washed with brine (60 mL), dried over Na₂SO₄, concentrated under vacuum and purified by flash column chromatography (0-20% MeOH) on Al₂O₃ to give compound 22-1 as s white foam solid (1.2 g, 69% yield).

Mass calc. C₁₂₆H₁₈₅N₁₁O₃₉ for: 2476.28; found: 1088.2 [M-DMTr+2H]²⁺, ESI.

¹H NMR (400 MHZ, DMSO-d₆) δ 7.85-7.81 (m, 3H), 7.75-7.72 (m, 3H), 7.47 (s, H), 7.39-7.37 (m, 2H), 7.32-7.25 (m, 2H), 7.24-7.19 (m, 5H), 6.88 (d, J=8.84 Hz, 4H), 5.22 (d, J=3.4 Hz, 2H), 4.97 (d, J=11.2 Hz, 3H), 4.75 (t, J=4.4 Hz, 1H), 4.50-4.48 (m, 5H), 4.03 (s, 11H), 3.94-3.80 (m, 7H), 3.77-3.66 (m, 9H), 3.50-3.41 (m, 6H), 3.02-3.00 (m, 12H), 2.92-2.79 (m, 2H), 2.43-2.30 (m, 2H), 2.29-2.22 (m, 2H), 2.21-2.19 (m, 3H), 2.11-2.08 (m, 11H), 2.05-2.02 (m, 8H), 2.00 (s, 9H), 1.90 (s, 9H), 1.78 (s, 9H), 1.62-1.23 (m, 48H).

### Example 23

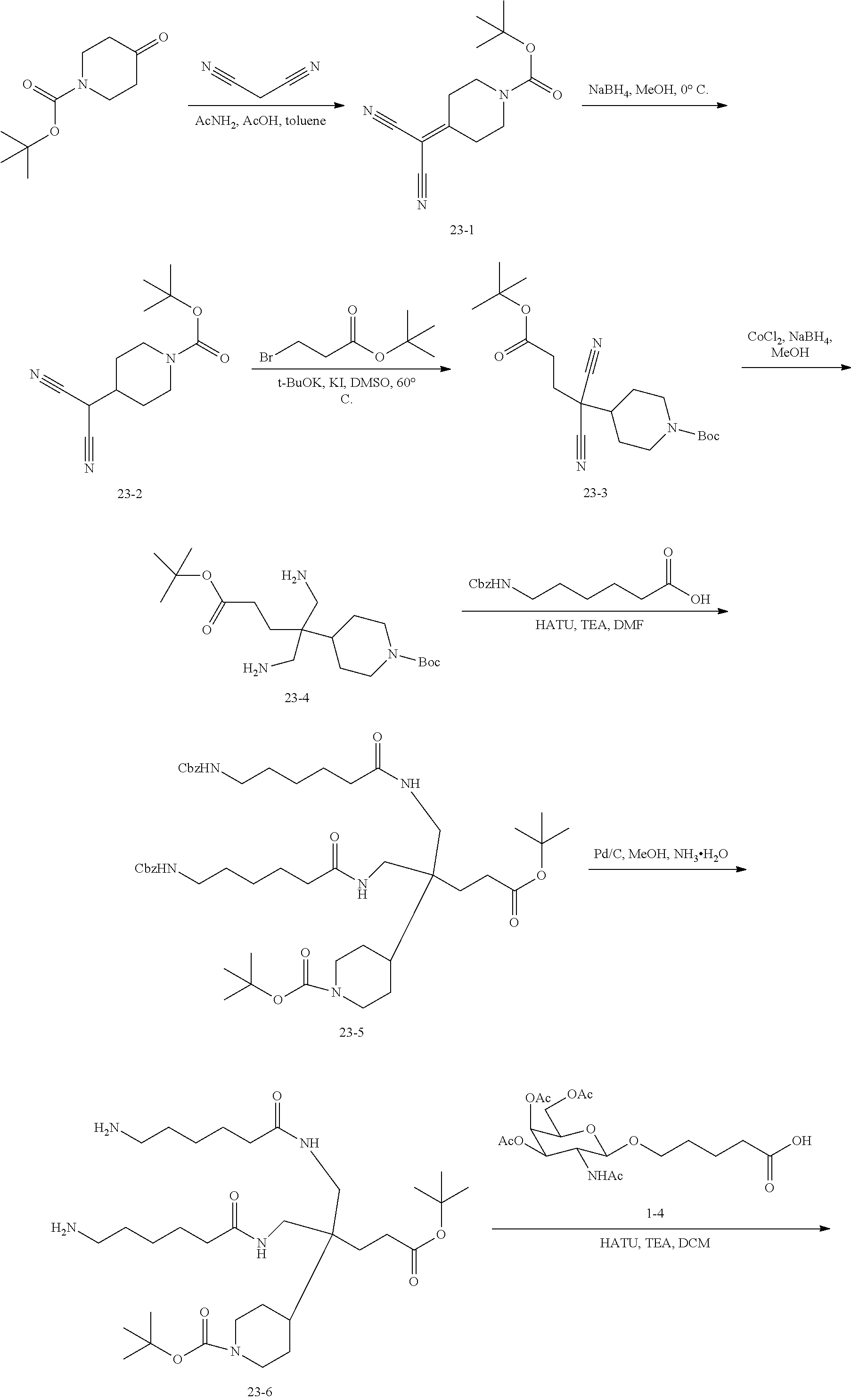

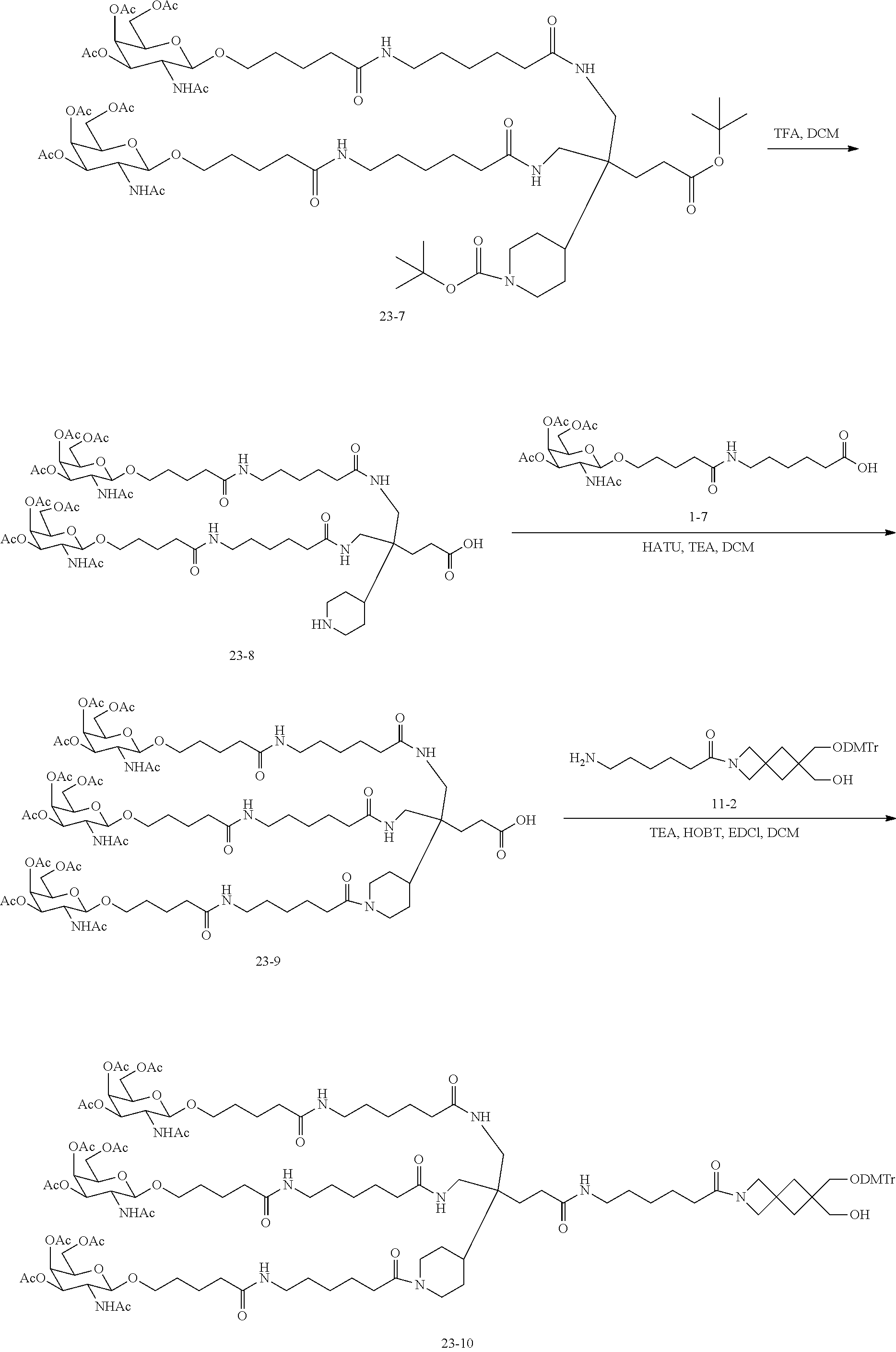

### Compound 23-1:

A solution of N-Boc-4-piperidone (5 g, 25.09 mmol), malononitrile (2.49 g, 37.64 mmol), AcNH₂ (3.87 g, 50.19 mmol) and AcOH (4.52 g, 75.28 mmol) in toluene (50 mL) was stirred at 110° C. for 2 h. LC-MS showed full conversion. The reaction mixture was washed by water (50 mL), dried over Na₂SO₄, concentrated and purified by silica gel column (0-10% EA in PE) to afford compound 23-1 as a gum (6.18 g, 98% yield).

Mass calc. for C₁₃H₁₇O₂N₃: 247.1; found: 246.2 [M−H], ESI.

¹H NMR (400 MHZ, DMSO-d₆) δ 3.53 (t, J=11.6 Hz, 4H), 2.69 (d, J=11.6 Hz, 4H), 7.41 (t, J=7.3 Hz, 2H), 1.427 (s, 9H).

### Compound 23-2:

A solution of compound 23-1 (7.6 g, 30.73 mmol) in MeOH (76 mL) was stirred at 0° C. NaBH₄ (1.16 g, 30.73 mmol) was added in portions to the stirred solution and stirred for 20 min. The mixture was concentrated in vacuo. The residue was partitioned between ethyl acetate (250 mL) and water (150 mL) and the ethyl acetate extract was washed with brine (100 mL), dried over sodium sulfate and concentrated in vacuo. The residue was purified by column chromatography eluting with PE/EA=1/5 to afford compound 23-2 as a gum (4.6 g, 60% yield).

Mass calc. for C₁₃H₁₉O₂N₃: 249.2; found: 248.3 [M−H], ESI.

¹H NMR (400 MHZ, DMSO-d₆) δ 4.04-4.01 (m, 2H), 2.75 (s, 2H), 2.31-2.27 (m, 1H), 1.82-1.78 (m, 2H), 1.04 (s, 9H), 1.27-1.16 (m, 3H).

### Compound 23-3:

A mixture of compound 23-2 (25 g, 100.28 mmol), tert-butyl 3-bromopropanoate (41.93 g, 200.56 mmol), t-BuOK (22.50 g, 200.56 mmol) and KI (33.29 g, 200.56 mmol) in DMSO (250 mL) was stirred at 60° C. overnight. LC-MS showed full conversion. The mixture was concentrated in vacuo. The residue was partitioned between ethyl acetate (100 mL×3) and water (150 mL×3) and the ethyl acetate extract was washed with brine (100 mL), dried over sodium sulfate and concentrated in vacuo. The residue was purified by column chromatography eluting with PE/EA=10/1 to afford compound 23-3 as a gum (32.87 g, 87% yield).

Mass calc. for C₂₀H₃₁O₄N₃: 377.2; found: 319.4 [M−Boc+MeCN+H]⁺, ESI. —¹H NMR (400 MHZ, DMSO-d₆) δ 4.06 (d, J=11.2 Hz, 2H), 2.75 (s, 2H), 2.55-2.53 (m, 1H), 2.35-2.31 (m, 3H), 1.89 (d, J=12.4 Hz, 2H), 1.43 (s, 9H), 1.41 (s, 9H), 1.27-1.19 (m, 3H).

### Compound 23-4:

Cobalt chloride hexahydrate (40.34 g, 169.55 mmol) was added to a solution of compound 23-3 (16 g, 42.39 mmol) in MeOH (400 mL), then sodium borohydride (32.07 g, 847.73 mmol) was slowly added to the reaction mixture at −20° C. and stirred for 1 h. Then the mixture was stirred for 1 h at room temperature. The reaction mixture was concentrated to dryness and azeotropically dried with DMF to give the crude compound 23-4, which was directly used for the next step without further purification.

Mass calc. for C₂₀H₃₉O₄N₃: 385.5; found: 386.5 [M+H]⁺, ESI.

### Compound 23-5:

A solution of Z-6-Aminohexanoic acid (27.53 g, 103.75 mmol), TEA (12.6 g, 124.50 mmol), HATU (46.97 g, 124.50 mmol) in DMF (240 mL) was stirred at room temperature for 30 min, compound 23-4 (16 g, 41.50 mmol) was added and stirred at rt overnight. LC-MS showed full conversion. Water (50 mL) and EA (50 mL) was added to the reaction mixture. The mixture was filtered and the filtrate was partitioned between ethyl acetate (100 mL×3) and water (150 mL×3) and the ethyl acetate extract was washed with brine (100 mL), dried over sodium sulfate and concentrated in vacuo. The residue was purified by prep-HPLC (C18 column, ACN/water) to afford compound 23-5 as a gum (8.3 g, 23% yield).

Mass calc. for C₄₈H₇₂O₁₁N₄: 879.5; found: 880.1 [M+H]⁺, ESI.

¹H NMR (400 MHZ, DMSO-d₆) δ 7.73 (t, J=12.4 Hz, 2H), 7.38-7.30 (m, 10H), 7.23 (t, J=10.8 Hz, 2H), 5.00 (s, 4H), 4.06-3.60 (m, 4H), 3.00-2.97 (m, 8H), 2.23-2.19 (m, 4H), 1.99 (s, 2H), 1.58-1.48 (m, 6H), 1.42-1.38 (m, 25H), 1.25-1.23 (m, 4H).

### Compound 23-6:

A mixture of compound 23-5 (3.8 g, 4.31 mmol), NH₃·H₂O (3.5 mL) and Pd/C (760 mg) in MeOH (38 mL) was stirred at room temperature for 2 h under hydrogen atmosphere. LC-MS showed full conversion. The mixture was filtered and the filtrate was concentrated in vacuo to give the crude compound 23-6, which was used directly for the next step without further purification.

Mass calc. for C₃₂H₆₀N₄O₇: 611.8; found: 612.8 [M+H]⁺, ESI.

### Compound 23-7:

A mixture of compound 1-4 (2.46 g, 5.49 mmol), TEA (757.89 mg, 7.49 mmol) and HATU (2.35 mg, 6.24 mmol) in DCM (15 mL) was stirred for 30 min at room temperature. Compound 23-6 (1.53 g, 2.5 mmol) was added and the mixture was stirred at rt overnight. LC-MS showed full conversion. The reaction mixture was diluted by adding DCM (20 mL), then washed with water (50 mL), concentrated and purified by prep-HPLC (C18 column, ACN/water) to afford compound 23-7 (3.14 g, 85% yield).

¹H NMR (400 MHZ, DMSO-d₆) δ 7.82 (d, J=9.2 Hz, 2H), 7.71 (s, 4H), 5.52 (d, J=0.4 Hz, 2H), 4.99-4.95 (m, 2H), 4.48 (d, J=8.8 Hz, 2H), 4.03 (s, 8H), 3.95-3.84 (m, 2H), 3.72-3.70 (m, 2H), 3.44-3.38 (m, 2H), 3.01-2.99 (m, 8H), 2.23-2.19 (m, 2H), 2.11-2.07 (m, 10H), 2.05-2.00 (m, 10H), 1.90 (s, 6H), 1.78 (s, 6H), 1.58-1.49 (m, 14H), 1.39-1.38 (m, 25H), 1.24-1.23 (m, 8H).

### Compound 23-8:

A solution of compound 23-7 (3.0 g, 2.04 mmol) and TFA (12 mL) in DCM (30 mL) was stirred at room temperature for 4 h. LC-MS showed full conversion. The solution was concentrated in vacuo and then basified by adding TEA. The compound 23-8 was used directly for the next step without further purification.

Mass calc. for C₆₁H₉₉O₂₄N₇: 1313.4; found: 1314.4 [M+H]⁺, ESI.

Compound 23-9: Compound 1-7 (2.68 g, 2.04 mmol), TEA (412.62.89 mg, 4.08 mmol) and HATU (769.19 mg, 2.04 mmol) in DCM (30 mL) was stirred for 4 h at room temperature. Then the solution was added into compound 23-8 (2.68 g, 2.04 mmol) and stirred at rt overnight. LC-MS showed full conversion. The reaction mixture was diluted by adding DCM (20 mL), washed with water (50 mL), concentrated and purified by prep-HPLC (C18 column, ACN/water) to afford compound 23-9 (1.76 g, 47% yield).

Mass calc. for C₈₆H₁₃₇O₃₅N₉: 1857.0; found: 929.0 [M+2H]²⁺, ESI.

¹H NMR (400 MHZ, DMSO-d₆) δ 7.94 (d, J=9.2 Hz, 2H), 7.88-7.79 (m, 6H), 5.21 (d, J=3.2 Hz, 3H), 4.99-4.95 (m, 3H), 4.51-4.48 (m, 4H), 4.05-4.00 (m, 9H), 3.92-3.84 (m, 4H), 3.73-3.37 (m, 3H), 2.99 (s, 10H), 2.81 (t, J=23.2 Hz, 1H), 2.48-2.44 (m, 2H), 2.34-2.16 (m, 6H), 2.11-2.09 (m, 13H), 2.05-2.00 (m, 16H), 1.89 (s, 9H), 1.78 (s, 9H), 1.49-1.35 (m, 31H), 1.24-1.23 (m, 6H).

### Compound 23-10:

A mixture of compound 23-9 (1.76 g, 0.94 mmol), DIEA (367.47 mg, 2.84 mmol), HOBT (256.12 mg, 1.90 mmol) and EDCI (363.37 mg, 1.90 mmol) in DCM (20 mL) was stirred at 0° C. for 1 h, then compound 11-2 (542.80 mg, 0.94 mmol) was added.

The mixture was stirred at rt overnight. LC-MS showed full conversion. The reaction mixture was diluted by adding DCM (20 mL), washed with NaHCO₃ (sat. aq., 50 mL), and water (50 mL), concentrated and purified by prep-HPLC (C18 column, ACN/water) to afford compound 23-10 (1.07 g, 47% yield).

¹H NMR (400 MHZ, DMSO-d₆) δ 7.83 (d, J=9.2 Hz, 3H), 7.76-7.65 (m, 6H), 7.40-7.30 (m, 4H), 7.26-7.22 (m, 5H), 6.92-6.89 (m, 4H), 5.22 (d, J=0.8 Hz, 3H), 4.99-4.95 (m, 3H), 4.65 (d, J=10.4 Hz, 1H), 4.49 (d, J=8.4 Hz, 4H), 4.03-3.98 (m, 10H), 3.91-3.89 (m, 4H), 3.74-3.67 (m, 11H), 3.51 (s, 1H), 3.41-3.39 (m, 5H), 3.01-2.92 (m, 14H), 2.27-2.25 (m, 14H), 2.05-2.02 (m, 7H), 2.00-1.94 (m, 12H), 1.78 (s, 9H), 1.49-1.37 (m, 33H), 1.24-1.22 (m, 11H).

### Example 24

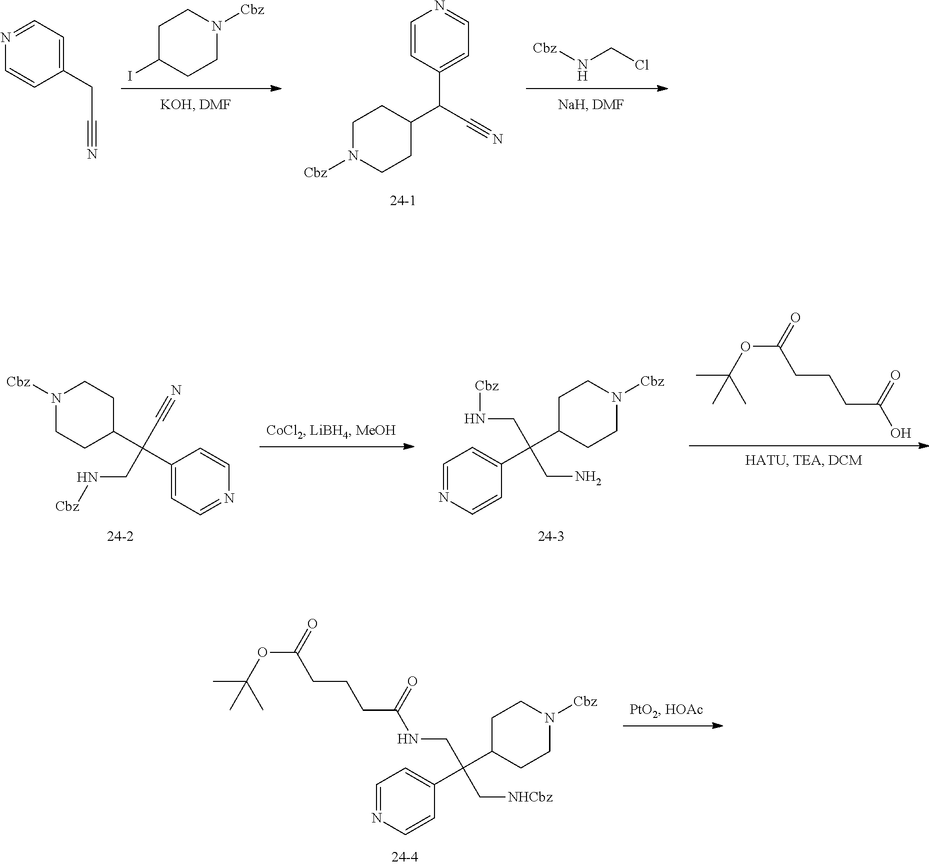

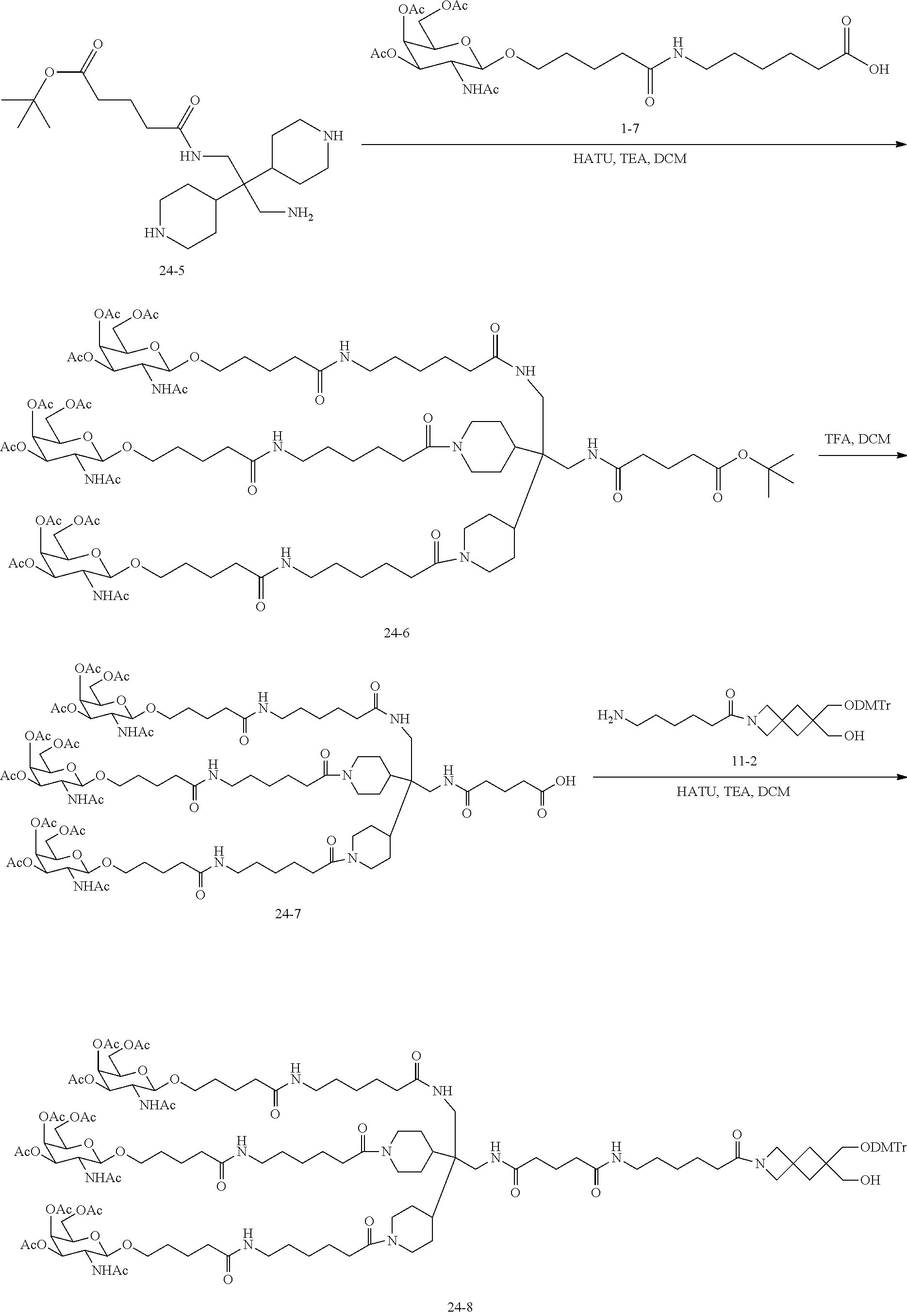

### Compound 24-1:

4-Pyridylacetonitrile (5 g, 42.32 mmol), N-Cbz-4-iodopiperidine (21.91 g, 63.49 mmol) and KOH (4.75 g, 84.64 mmol) were dissolved in DMF (50 mL). The reaction mixture was stirred at rt overnight. LC-MS showed full conversion. The residue was partitioned between ethyl acetate (100 mL×2) and water (80 mL×3) and the ethyl acetate extract was washed with brine (100 mL), dried over sodium sulfate and concentrated in vacuo. The residue was purified by column chromatography eluting with DCM/MeOH=20/1 to afford compound 24-1 as a pale brown oil (11.3 g, 80% yield).

Mass calc. for C₂₀H₂₁N₃O₂: 335.4; found: 336.2 [M+H]⁺, ESI.

¹H NMR (400 MHZ, DMSO-d₆) δ 8.62-8.61 (m, 2H), 7.40-7.31 (m, 7H), 5.06 (s, 2H), 4.35 (d, J=7.2 Hz, 1H), 4.34-4.00 (m, 2H), 2.75 (s, 2H), 2.12-2.06 (m, 1H), 1.64-1.53 (m, 2H), 1.24-1.14 (m, 2H).

### Compound 24-2:

KI (2.97 g, 17.89 mmol) and NaH (858.6 mg, 35.78 mmol) in anhydrous THF (10 mL) under N₂ atmosphere was cooled at 0° C. for 10 min. Compound 24-1 (3 g, 8.94 mmol) in anhydrous THF (15 mL) was added. After the mixture was stirred for 30 min. Then benzyl N-(chloromethyl) carbamate (2.14 g, 10.73 mmol) in anhydrous THF (5 mL) was added and stirred at room temperature for overnight under N₂ atmosphere. The mixture was quenched with water and extracted with ethyl acetate (50 mL×3). The combined organic layers were washed with brine (60 mL), dried over anhydrous MgSO₄, concentrated in vacuo. The residue was purified by column chromatograph eluting with dichloromethane/methanol 4:1 to afford compound 24-2 as a yellow oil (1.8 g, 40% yield).

Mass calc. for C₂₉H₃₀N₄O₄: 498.2; found: 499.2 [M+H]⁺, ESI.

¹H NMR (400 MHZ, DMSO) δ 8.61 (d, J=5.2 Hz, 2H), 7.52-7.57 (m, 1H), 7.44-7.22 (m, 12H), 5.06 (s, 2H), 5.03-4.91 (m, 2H), 4.14 (d, J=13.6 Hz, 1H), 3.97-3.89 (m, 2H), 3.73-3.68 (m, 1H), 2.89-2.68 (m, 2H), 2.39-2.34 (t, J=11.6 Hz, 1H), 2.07 (d, J=11.8 Hz, 2H), 1.31-1.21 (m, 1H), 1.18-1.12 (m, 1H), 1.04-0.94 (m, 1H).

### Compound 24-3:

CoCl₂·6H₂O (2.97 g, 17.89 mmol) and compound 24-2 (5 g, 10.03 mmol) in anhydrous MeOH (50 mL) under N₂ atmosphere were cooled at −50° C. for 20 min. LiBH₄ in THF (50 mmol, 2M, 25 mL) was added dropwise at −50° C. and stirred for 3 h, then anhydrous MeOH (50 mL) was added dropwise at −50° C. The mixture was allowed to warm to room temperature and stirred for overnight under N₂ atmosphere. The mixture was concentrated in vacuo. The residue was partitioned between dichloromethane (250 mL) and water (150 mL) and the dichloromethane extract was washed with brine (100 mL), dried over sodium sulfate and concentrated in vacuo.

The residue was purified by column chromatography eluting with DCM/MeOH=15/1 to afford compound 24-3 as a pale-yellow solid (1.8 g, 40% yield).

Mass calc. for C₂₉H₃₄N₄O₄: 502.2; found: 503.2 [M+H]⁺, ESI.

¹H NMR (400 MHZ, DMSO-d₆) δ 8.48 (d, J=5.2 Hz, 2H), 7.50-7.47 (m, 1H), 7.37-7.25 (m, 12H), 5.02 (s, 2H), 4.99 (s, 2H), 3.96 (d, J=12.4 Hz, 2H), 3.69-3.58 (m, 2H), 3.16 (d, J=14 Hz, 1H), 2.94 (d, J=13.2 Hz, 1H), 2.63 (s, 2H), 1.93 (s, 2H), 1.77-1.68 (m, 2H), 1.26 (d, J=8.8 Hz, 1H), 0.86-0.77 (m, 2H).

### Compound 24-4:

Compound 24-3 (2.1 g, 4.18 mmol), 5-(tert-butoxy)-5-oxopentanoic acid (943.7 mg, 5.01 mmol), HATU (3.15 g, 8.36 mmol) and Et₃N (1.27 g, 12.53 mmol) were dissolved in DCM (20 mL). The reaction mixture was stirred at rt for 2 h. Then the reaction mixture was diluted with CH₂C12 (50 mL). The organic layer was washed with water (50 mL×2), dried over sodium sulfate, and concentrated in vacuo. The residue was purified by column chromatography eluting with DCM/MeOH=20/1 to afford compound 24-4 as a pale-yellow solid (2.23 g, 81% yield).

Mass calc. for C₃₈H₄₈N₄O₇: 672.3; found: 673.3 [M+H]⁺, ESI.

¹H NMR (400 MHZ, DMSO-d₆) δ 8.48 (d, J=5.2 Hz, 2H), 7.83-7.79 (m, 1H), 7.36-7.21 (m, 13H), 5.01 (s, 2H), 4.98 (s, 2H), 3.96 (d, J=12.4 Hz, 2H), 3.65-3.52 (m, 4H), 2.68-2.62 (m, 2H), 2.12-2.09 (m, 4H), 1.77-1.69 (m, 5H), 1.35 (s, 9H), 0.76-0.64 (m, 2H).

### Compound 24-5:

PtO₂ (1.21 g, 5.36 mmol) and compound 24-4 (1.8 g, 2.67 mmol) in HOAc (10 mL) under H₂ atmosphere was stirred at room temperature overnight. Then the mixture was filtered and the solvent was evaporated in vacuo to give the crude product 24-5, which was used directly for the next step.

Mass calc. for C₂₂H₄₂N₄O₃: 410.3; found: 411.3 [M+H]⁺, ESI.

### Compound 24-6:

A mixture of compound 1-7 (4.76 g, 8.52 mmol), TEA (1.48 g, 14.6 mmol), HATU (2.94 g, 7.8 mmol) in DCM (15 mL) was stirred for 30 min at room temperature, then compound 24-5 (1 g, 2.44 mmol) was added and stirred at rt for 2 h. LC-MS showed full conversion. The reaction mixture was diluted by additional DCM (20 mL), and washed by water (50 mL), concentrated and purified by prep-HPLC (C18 column, ACN/water) to afford compound 24-6 as a white solid (1 g, 20% yield).

Mass calc. for C₉₇H₁₅₆N₁₀O₃₆: 2037.07; found: 1019.1 [M+2H]²⁺, ESI.

¹H NMR (400 MHZ, DMSO-d₆) δ 7.80 (d, J=9.2 Hz, 3H), 7.73-7.63 (m, 6H), 5.22 (d, J=3.2 Hz, 3H), 4.97 (dd, J=11.2, 3.2 Hz, 3H), 4.50-4.47 (m, 5H), 4.08-4.01 (m, 9H), 3.91-3.86 (m, 5H), 3.84-3.70 (m, 3H), 3.43-3.40 (m, 3H), 3.07-3.05 (m, 3H), 2.81 (d, J=9.2 Hz, 2H), 2.68-2.67 (m, 1H), 2.34-2.23 (m, 12H), 2.18 (s, 9H), 2.16-2.11 (m, 5H), 2.03 (s, 9H), 1.90 (s, 9H), 1.78 (s, 9H), 1.72-1.67 (m, 14H), 1.54-1.48 (m, 18H), 1.43-1.37 (m, 13H), 1.28-1.18 (m, 12H).

### Compound 24-7:

A solution of compound 24-6 (1.0 g, 0.49 mmol) and TFA (12 mL) in DCM (30 mL) was stirred at room temperature for 4 h. LC-MS showed full conversion. The solution was concentrated in vacuo and then basified by TEA. The crude compound 24-7 was directly used for the next step without further purification.

Mass calc. for C₉₃H₁₄₈N₁₀O₃₆: 1981.0; found: 991.4 [M+2H]²⁺, ESI.

### Compound 24-8:

A mixture of compound 24-7 (400 mg, 0.21 mmol), TEA (61.26 mg, 0.61 mmol), HATU (252.26 mg, 0.40 mmol) in DCM (5 mL) was stirred at rt for 1 h, then compound 11-2 (115.16 mg, 0.20 mmol) was added and stirred at rt overnight. LC-MS showed full conversion. The reaction mixture was diluted by additional DCM (5 mL), and washed by water (20 mL), concentrated and purified by prep-HPLC (C18 column, ACN/water) to afford compound 24-8 as a white solid (200 mg, 39% yield).

¹H NMR (400 MHz, DMSO-d₆) δ 7.83 (d, J=9.2 Hz, 3H), 7.76-7.66 (m, 5H), 7.40-7.30 (m, 4H), 7.26-7.22 (m, 5H), 6.92-6.88 (m, 4H), 5.22 (d, J=3.2 Hz, 3H), 4.99-4.95 (m, 3H), 4.66 (t, J=5.6 Hz, 1H), 4.48 (d, J=8.4 Hz, 4H), 4.43-4.42 (m, 1H), 4.05-3.97 (m, 9H), 3.91-3.86 (m, 5H), 3.74-3.62 (m, 11H), 3.51 (s, 1H), 3.43-3.38 (m, 5H), 3.06-2.92 (m, 13H), 2.86-2.82 (m, 2H), 2.34-2.22 (m, 6H), 2.11 (s, 9H), 2.08-2.06 (m, 3H), 2.05-2.02 (m, 8H), 2.00 (s, 9H), 1.96-1.94 (m, 3H), 1.89 (s, 9H), 1.85-1.82 (m, 3H), 1.78 (s, 9H), 1.72-1.62 (m, 5H), 1.52-1.42 (m, 18H), 1.39-1.33 (m, 8H), 1.28-1.22 (m, 9H), 0.95 (t, J=8.4 Hz, 1H).

### Example 25

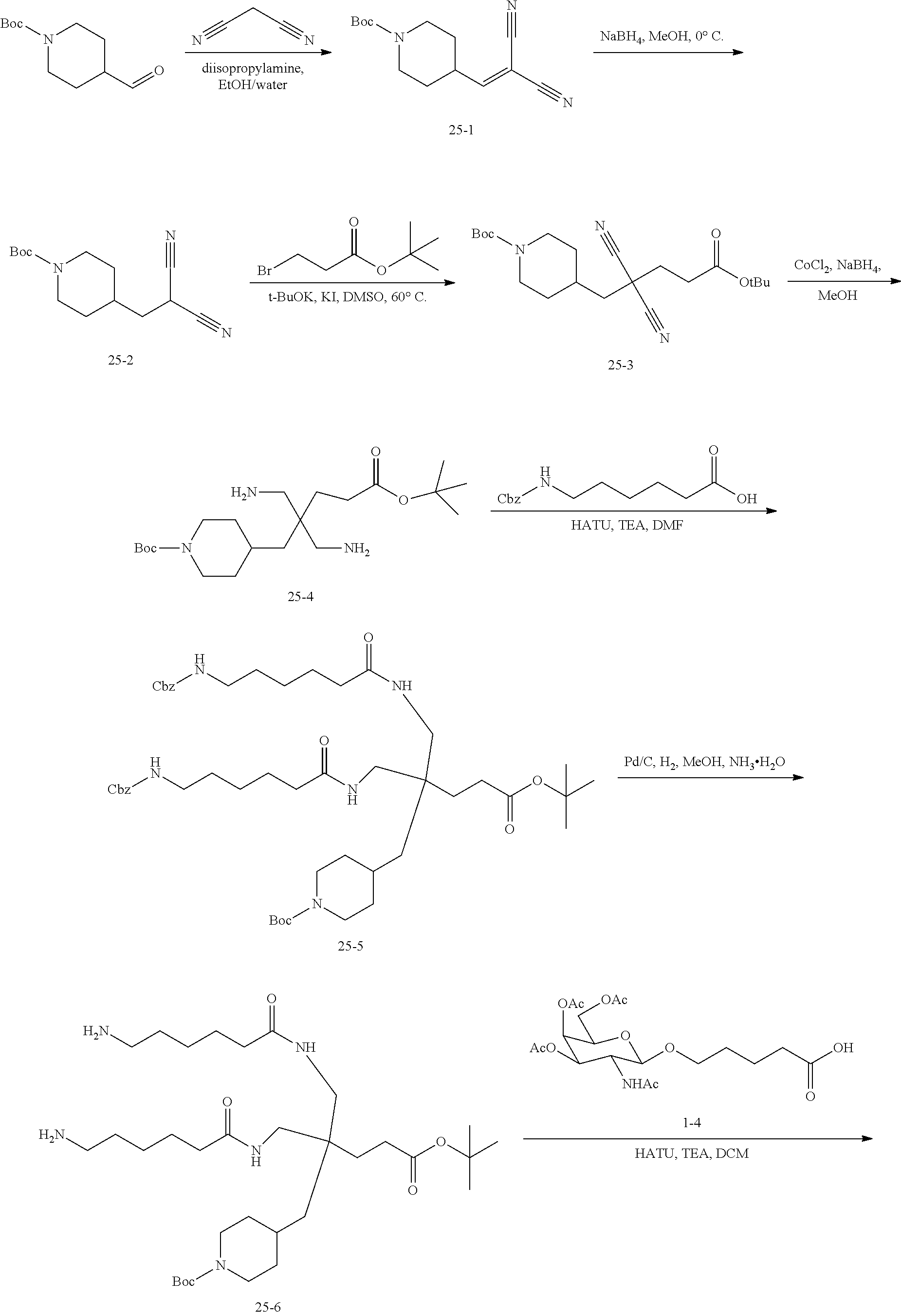

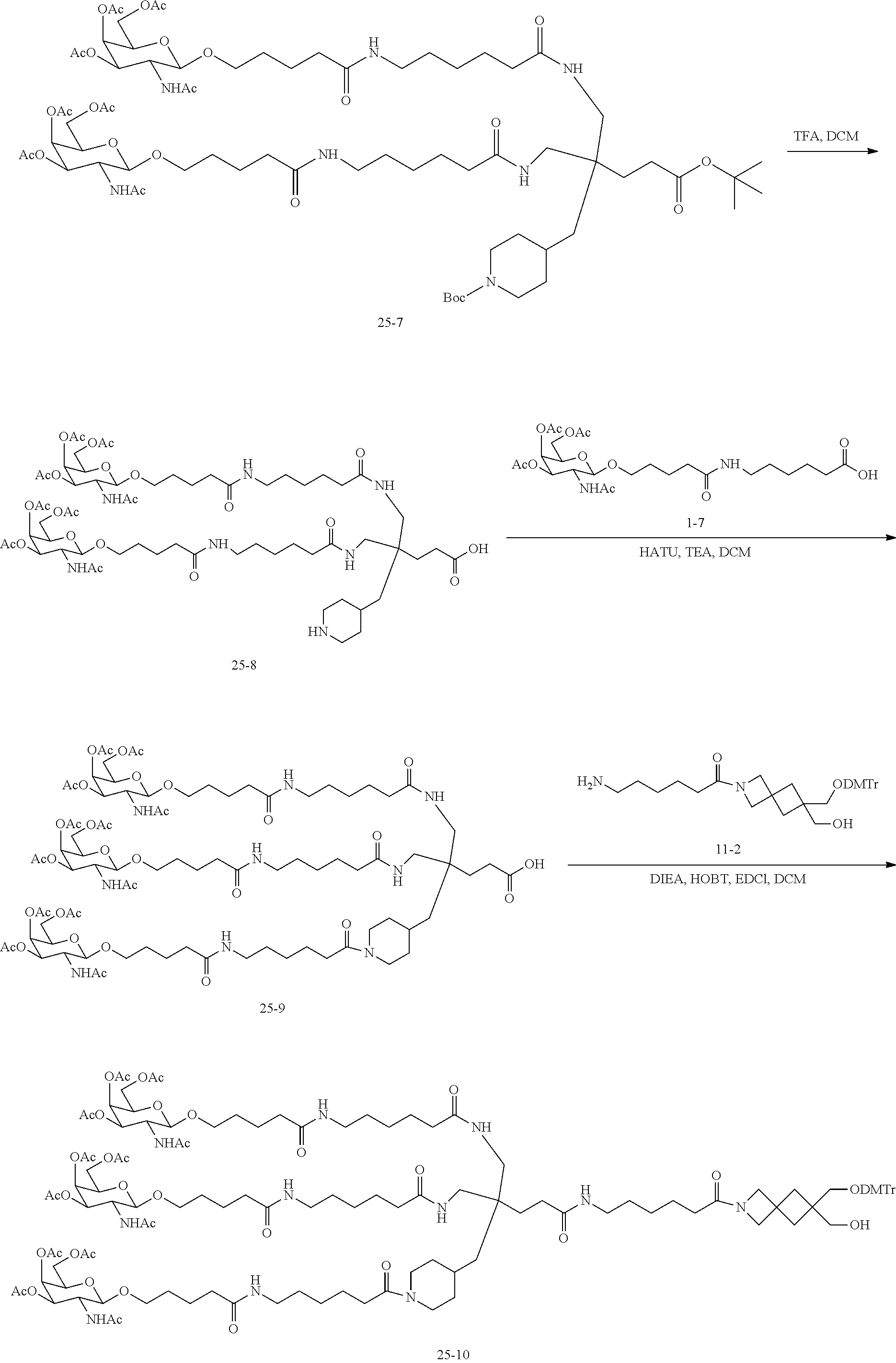

### Compound 25-1:

A solution of N-Boc-piperidine-4-carboxaldehyde (50 g, 234.44 mmol), malononitrile (18.59 g, 281.33 mmol) and diisopropylamine (11.86 g, 117.21 mmol) in H₂O (200 mL) and EtOH (800 mL) was stirred at rt for 2 h. LC-MS showed full conversion. The reaction mixture was washed by water (50 mL), dried over Na₂SO₄, concentrated and purified by silica gel column (0-50% EA in PE) to afford compound 25-1 (30.00 g, 46% yield).

Mass calc. for C₁₄H₁₉N₃O₂: 261.1; found: 260.2 [M−H], ESI.

¹H NMR (400 MHZ, DMSO-d₆) δ 7.84 (d, J=10.2 Hz, 1H), 3.97-3.89 (m, 2H), 2.94-2.72 (m, 3H), 1.69-1.59 (m, 2H), 1.45-1.34 (m, 13H).

### Compound 25-2:

Compound 25-1 (30 g, 114.80 mmol) in MeOH (450 mL) was stirred at −5° C. NaBH₄ (4.34 g, 114.80 mmol) was add in portions to the solution and stirred for 3 h. The mixture was concentrated in vacuo. The residue was partitioned between ethyl acetate (250 mL) and water (150 mL), and the ethyl acetate extract was washed with brine (100 mL), dried over sodium sulfate and concentrated in vacuo. The residue was purified by column chromatography eluting with PE/EA=3/1 to afford compound 25-2 (19.4 g, 64% yield).

Mass calc. for C₁₄H₂₁N₃O₂: 263.2; found: 262.3 [M−H], ESI.

¹H NMR (400 MHZ, DMSO-d₆) δ 4.87 (t, J=7.6 Hz, 1H), 3.95-3.87 (m, 2H), 2.82-2.61 (m, 2H), 1.98 (t, J=7.2 Hz, 2H), 1.70-1.56 (m, 3H), 1.42-1.35 (m, 9H), 1.11-0.98 (m, 2H).

### Compound 25-3:

A mixture of compound 25-2 (15 g, 56.96 mmol), tert-butyl 3-bromopropanoate (17.86 g, 85.44 mmol), t-BuOK (8.31 g, 74.05 mmol) and KI (9.46 g, 56.96 mmol) in DMSO (250 mL) was stirred at rt for 3 h. LC-MS showed full conversion. The mixture was partitioned between ethyl acetate (100 mL×3) and water (150 mL×3) and the ethyl acetate extract was washed with brine (100 mL), dried over sodium sulfate and concentrated in vacuo. The residue was purified by column chromatography eluting with PE/EA=7/1 to afford compound 25-3 (17.4 g, 94% yield).

Mass calc. for C₂₁H₃₃N₃O₄: 391.2; found: 333.3 [M−Boc+MeCN+H]⁺, ESI.

¹H NMR (400 MHz, DMSO-d₆) δ 3.96-3.87 (m, 2H), 2.76 (s, 2H), 2.55-2.50 (m, 2H), 2.36-2.31 (m, 2H), 2.05 (d, J=6.2 Hz, 2H), 1.85-1.74 (m, 3H), 1.42 (s, 9H), 1.39 (s, 9H), 1.21-1.08 (m, 2H).

### Compound 25-4:

Cobalt chloride hexahydrate (29.17 g, 122.60 mmol) was added to a solution of compound 25-3 (12 g, 30.65 mmol) in MeOH (350 mL), then sodium borohydride (23.19 g, 613.02 mmol) was slowly added to the reaction mixture at −20° C. and stirred for 1 h. Then the mixture was stirred for 1 h at room temperature. The reaction mixture was concentrated to dryness and azeotropically dried with DMF. The crude compound 25-4 was directly used for the next step without further purification.

Mass calc. for C₂₁H₄₁N₃O₄: 399.3; found: 400.4 [M+H]⁺, ESI.

### Compound 25-5:

A solution of Z-6-aminohexanoic acid (17.52 g, 66.04 mmol), TEA (9.10 g, 90.05 mmol) and HATU (28.31 g, 75.04 mmol) in DMF (240 mL) was stirred at room temperature for 30 min, compound 25-4 (12 g, 30.03 mmol) was added and stirred at rt overnight. LC-MS showed full conversion. Water (50 mL) and EA (50 mL) was added to the reaction mixture. The mixture was filtered and the filtrate was partitioned between ethyl acetate (100 mL×3) and water (150 mL×3) and the ethyl acetate extract was washed with brine (100 mL), dried over sodium sulfate and concentrated in vacuo. The residue was purified by prep-HPLC (C18 column, ACN/water) to afford compound 25-5 (4.08 g, 14% yield).

Mass calc. for C₄₉H₇₅N₅O₁₀: 893.6; found: 894.7 [M+H]⁺, ESI.

¹H NMR (400 MHZ, DMSO-d₆) δ 7.68 (t, J=6.1 Hz, 2H), 7.38-7.27 (m, 10H), 7.22 (t, J=5.5 Hz, 2H), 4.99 (s, 4H), 3.89-3.79 (m, 2H), 3.00-2.93 (m, 4H), 2.92-2.85 (m, 3H), 2.73-2.56 (m, 2H), 2.23-2.16 (m, 2H), 2.13-2.06 (m, 4H), 1.61-1.42 (m, 9H), 1.42-1.34 (m, 24H), 1.28-1.18 (m, 4H), 1.05-0.99 (m, 2H).

### Compound 25-6:

A mixture of compound 25-5 (3.0 g, 3.36 mmol), NH₃·H₂O (15 mL) and Pd/C (1.20 g) in MeOH (60 mL) was stirred at room temperature for 4 h under a hydrogen atmosphere. LC-MS showed full conversion. The mixture was filtered and the filtrate was concentrated in vacuo. The crude compound 25-6 was directly used for the next step without further purification.

Mass calc. for C₃₃H₆₃N₅O₆: 625.5; found: 626.5 [M+H]⁺, ESI.

### Compound 25-7:

A mixture of compound 1-4 (3.30 g, 7.38 mmol), TEA (1.02 g, 10.07 mmol) and HATU (3.04 g, 8.05 mmol) in DCM (15 mL) was stirred for 30 min at room temperature. Compound 25-6 (2.10 g, 3.36 mmol) was added and stirred at rt overnight. LC-MS showed full conversion. The reaction mixture was diluted by adding DCM (20 mL), and washed by water (50 mL), concentrated and purified by prep-HPLC (C18 column, ACN/water) to afford compound 25-7 (3.14 g, 86% yield).

¹H NMR (400 MHZ, DMSO-d₆) δ 7.82 (d, J=9.3 Hz, 2H), 7.74-7.65 (m, 4H), 5.21 (d, J=3.4 Hz, 2H), 4.99-4.93 (m, 2H), 4.48 (d, J=8.5 Hz, 2H), 4.05-3.98 (m, 6H), 3.91-3.80 (m, 4H), 3.74-3.67 (m, 2H), 3.44-3.36 (m, 2H), 3.03-2.95 (m, 4H), 2.91-2.86 (m, 3H), 2.73-2.60 (m, 2H), 2.24-2.17 (m, 2H), 2.13-2.07 (m, 10H), 2.05-1.98 (m, 10H), 1.89 (s, 6H), 1.71 (s, 6H), 1.61-1.32 (m, 41H), 1.27-1.19 (m, 4H), 1.04-0.99 (m, 2H).

### Compound 25-8:

A solution of compound 25-7 (1.0 g, 673.53 μmol) and TFA (10 mL) in DCM (20 mL) was stirred at room temperature for 4 h. LC-MS showed full conversion. The solution was concentrated in vacuo and then basified by TEA. The crude compound 25-8 was directly used for the next step without further purification.

Mass calc. for C₆₂H₁₀₁N₇O₂₄: 1327.7; found: 665.0 [M+2H]²⁺, ESI.

### Compound 25-9:

A mixture of compound 1-7 (1.43 g, 2.56 mmol), TEA (777 mg, 7.68 mmol) and HATU (1.06 g, 2.82 mmol) in DCM (30 mL) was stirred for 30 min at room temperature. Then the solution was added into compound 22-8 (3.4 g, 2.56 mmol) and stirred at rt overnight. LC-MS showed full conversion. The reaction mixture was diluted by adding DCM (20 mL), washed by water (50 mL), concentrated and purified by prep-HPLC (C18 column, ACN/water) to afford compound 25-9 (2.5 g, 49% yield).

Mass calc. for C₈₇H₁₃₉N₉O₃₅: 1869.9; found: 936.3 [M+2H]²⁺, ESI.

¹H NMR (400 MHZ, DMSO-d₆) δ 7.96-7.90 (m, 2H), 7.87-7.75 (m, 5H), 7.71 (s, 1H), 5.21 (d, J=3.4 Hz, 3H), 4.99-4.94 (m, 3H), 4.52-4.47 (m, 3H), 4.32-4.25 (m, 1H), 4.05-3.98 (m, 9H), 3.92-3.82 (m, 3H), 3.80-3.67 (m, 5H), 3.52-3.42 (m, 4H), 3.03-2.91 (m, 10H), 2.89-2.80 (m, 2H), 2.24 (t, J=7.5 Hz, 2H), 2.20-2.13 (m, 2H), 2.13-2.07 (m, 13H), 2.05-1.96 (m, 15H), 1.89 (s, 9H), 1.77 (s, 9H), 1.68-1.55 (m, 4H), 1.54-1.32 (m, 26H), 1.28-1.18 (m, 6H), 1.04-0.98 (m, 2H).

### Compound 25-10:

A mixture of compound 25-9 (750 mg, 400.84 μmol), DIEA (155 mg, 1.20 mmol), HOBT (217.12 mg, 1.60 mmol) and EDCI (307.36 mg, 1.06 mmol) in DCM (20 mL) was stirred for 30 min, compound 11-2 (229.57 mg, 400.84 μmol) was added and stirred at rt for 3 h. LC-MS showed full conversion. The reaction mixture was diluted by adding DCM (20 mL), washed by NaHCO₃ (sat. aq., 30 mL) and water (30 mL), concentrated and purified by prep-HPLC (C18 column, ACN/water) to afford compound 25-10 (500 mg, 51% yield).

¹H NMR (400 MHZ, DMSO-d₆) δ 7.83 (d, J=9.2 Hz, 3H), 7.75-7.65 (m, 6H), 7.41-7.34 (m, 2H), 7.31 (t, J=7.1 Hz, 2H), 7.27-7.18 (m, 5H), 6.93-6.84 (m, 4H), 5.22 (d, J=3.0 Hz, 3H), 5.00-4.90 (m, 3H), 4.71-4.56 (m, 1H), 4.48 (d, J=8.4 Hz, 3H), 4.28 (d, J=10.7 Hz, 1H), 4.11-3.93 (m, 10H), 3.92-3.82 (m, 3H), 3.77-3.63 (m, 12H), 3.51 (s, 1H), 3.45-3.36 (m, 6H), 3.05-2.79 (m, 15H), 2.28-2.19 (m, 2H), 2.12-2.08 (m, 12H), 2.06-1.98 (m, 17H), 1.96-1.92 (m, 3H), 1.89 (s, 9H), 1.85-1.80 (m, 2H), 1.77 (s, 9H), 1.60-1.00 (m, 47H).

### Example 26

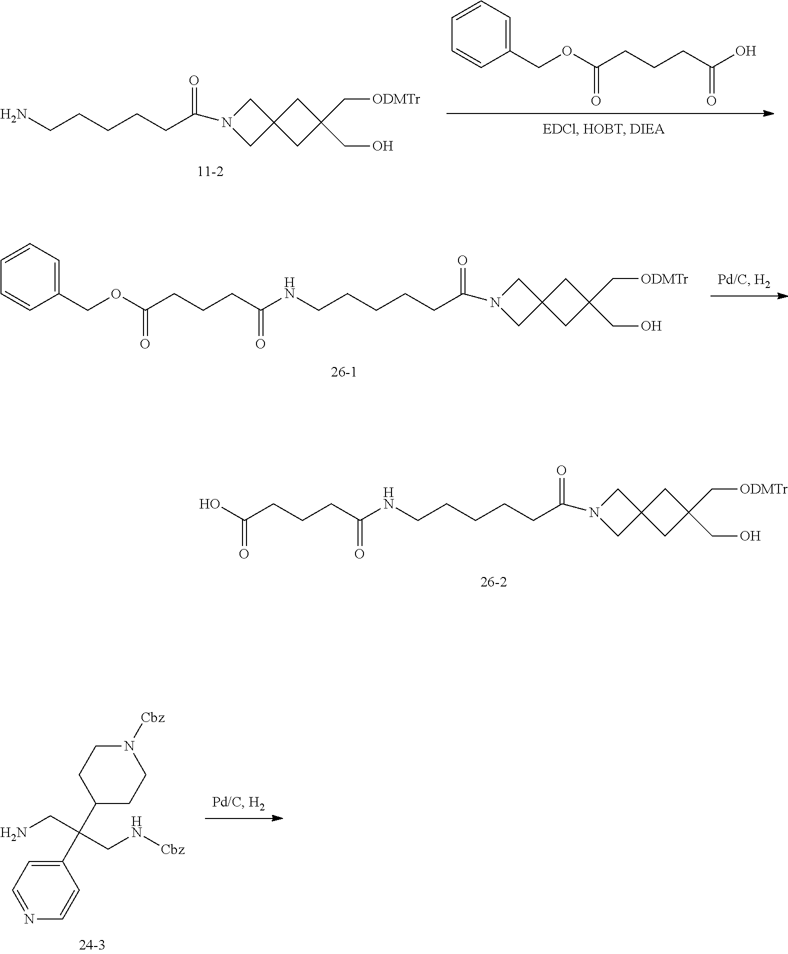

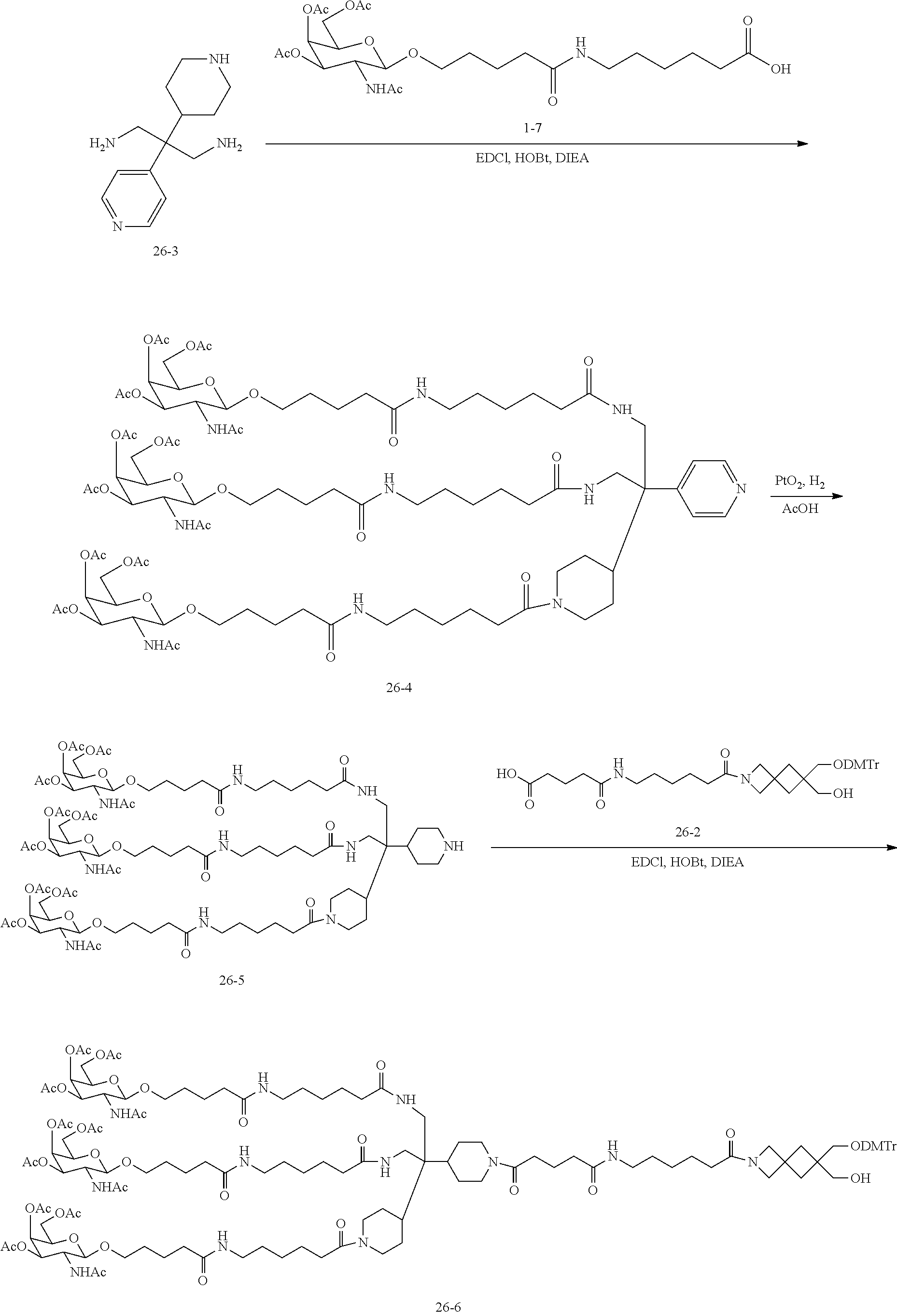

### Compound 26-1:

A mixture of 5-(benzyloxy)-5-oxopentanoic acid (0.3 g, 1.35 mmol), compound 11-2 (773.14 mg, 1.35 mmol), DIEA (523.40 mg, 4.05 mmol), EDC (517.56 mg, 2.70 mmol), HOBT (364.81 mg, 2.70 mmol) in DCM (20 mL) was stirred at rt for 3 h. LC-MS showed full conversion. The reaction mixture was diluted by adding DCM (20 mL), washed by NaHCO₃ (sat. aq., 150 mL) and water (150 mL), concentrated and purified by column chromatography eluting with DCM/MeOH=20:1 to afford compound 26-1 (480 mg, 45% yield).

Mass calc. for C₄₇H₅₆N₂O₈: 776.4; found: 799.3 [M+Na]⁺, ESI.

### Compound 26-2:

A mixture of compound 26-1 (0.48 g, 0.629 mmol) and Pd/C (48 mg) in MeOH (20 mL) stirred at rt for 3 h under H₂. LC-MS showed full conversion. The reaction mixture was concentrated and purified by column chromatography eluting with DCM/MeOH=10:1 to afford compound 26-2 (280 mg, 65% yield).

Mass calc. for C₄₇H₅₆N₂O₈: 686.3; found: 725.3 [M+K]⁺, ESI

¹H NMR (400 MHZ, DMSO-d₆) δ 7.79-7.75 (m, 1H), 7.40-7.35 (m, 2H), 7.34-7.30 (m, 2H), 7.27-7.20 (m, 5H), 6.92-6.89 (m, 4H), 3.98 (s, 1H), 3.68 (d, J=6.4 Hz, 2H), 3.51 (s, 1H), 3.40-3.38 (m, 2H), 3.00 (t, J=6.4 Hz, 2H), 2.93-2.92 (m, 2H), 2.19 (t, J=7.4 Hz, 2H), 2.09-2.05 (m, 2H), 2.03-1.88 (m, 4H), 1.85-1.80 (m, 2H), 1.71-1.68 (m, 2H), 1.42-1.33 (m, 4H), 1.24-1.18 (m, 4H).

### Compound 26-3:

A mixture of compound 26-2 (3.1 g, 6.17 mmol) and Pd/C (2.97 g) in anhydrous MeOH (40 mL) was stirred at room temperature overnight under H₂. The mixture was filtered and the filtrate was concentrated under reduce pressure to give the crude compound 26-3 as a white solid (1.4 g, 97% yield), which was used without purification.

### Compound 26-4:

A mixture of compound 26-3 (1.50 g, 6.40 mmol), compound 1-7 (11.12 g, 19.84 mmol), EDCI (7.34 g, 38.41 mmol), HOBT (5.19 g, 38.41 mmol), DIEA (7.45 g, 57.61 mmol) in anhydrous DCM (50 mL) was stirred at room temperature for 3 h under N₂ atmosphere. The mixture was concentrated in vacuo, then extracted with dichloromethane (100 mL×3) and washed with brine (100 mL), dried over sodium sulfate and concentrated in vacuo. The residue was purified by prep-HPLC (C18 column, ACN/water) to afford compound 26-4 as a pale-yellow solid (6 g, 50% yield).

Mass calc. for C₈₈H₁₃₆N₁₀O₃₃: 1860.9; found: 931.5 [M+2H]²⁺, ESI.

### Compound 26-5:

A mixture of compound 26-4 (1.01 g, 0.53 mmol) and PtO₂ (0.243 g, 1.07 mmol) in AcOH (20 mL) was stirred overnight under H₂. The reaction mixture was filtered and concentrated in vacuo. Then the residue was purified by prep-HPLC (C18 column, ACN/water) to afford compound 26-5 as a white solid (640 mg, 64% yield).

Mass calc. for C₈₈H₁₄₂N₁₀O₃₃: 1867.0; found: 934.5 [M+2H]²⁺, ESI.

¹H NMR (400 MHZ, DMSO-d₆) δ 7.86-7.84 (m, 3H), 7.75-7.73 (m, 3H), 7.68-7.61 (m, 2H), 5.22 (d, J=3.3 Hz, 3H), 4.97 (q, J=3.3 Hz, 3H), 4.50-4.43 (m, 4H), 4.03 (s, 9H), 3.91-3.84 (m, 4H), 3.75-3.70 (m, 3H), 3.43-3.38 (m, 11H), 3.06-2.99 (m, 12H), 2.83-2.77 (m, 1H), 2.46-2.42 (m, 2H), 2.27-2.23 (m, 4H), 2.11-2.09 (m, 13H), 2.07-2.00 (m, 16H), 1.90 (s, 9H), 1.78 (s, 9H), 1.68-1.62 (m, 5H), 1.49-1.46 (m, 22H), 1.39-1.36 (m, 9H), 1.25-1.20 (m, 9H).

### Compound 26-6:

A mixture of compound 26-5 (0.543 g, 0.29 mmol), compound 26-2 (200 mg, 2.12 mmol), DIEA (112 mg, 0.87 mmol), EDC (111 mg, 0.58 mmol) and HOBT (80 mg, 0.58 mmol) in DCM (20 mL) was stirred at rt for 3 h. LC-MS showed full conversion. The reaction mixture was diluted by adding DCM (20 mL), washed with NaHCO₃ (sat. aq., 150 mL) and water (150 mL), concentrated and purified by prep-HPLC (C18 column, ACN/water) to afford compound 26-6 (0.15 g, 20% yield).

Mass calc. for C₁₂₁H₁₇₉O₃₉N₁₁: 3411.7; found: 1117.2 [M-DMTr+2H]²⁺, ESI.

¹H NMR (400 MHZ, DMSO-d₆) δ 7.84 (d, J=9.3 Hz, 3H), 7.78-7.72 (m, 5H), 7.65 (s, 2H), 7.40-7.37 (m, 2H), 7.32 (t, J=7.1 Hz, 2H), 7.26-7.20 (m, 6H), 6.92-6.89 (m, 4H), 5.22 (d, J=3.4 Hz, 2H), 4.98-4.95 (m, 2H), 4.65 (t, J=5.0 Hz, 1H), 4.48 (d, J=8.4 Hz, 2H), 4.45-4.42 (m, 2H), 4.05-4.03 (m, 9H), 3.98 (s, 2H), 3.91-3.84 (m, 5H), 3.74 (s, 7H), 3.71-3.67 (m, 5H), 3.51 (s, 1H), 3.43-3.39 (m, 6H), 3.07-2.97 (m, 13H), 2.92 (s, 2H), 2.81-2.78 (m, 2H), 2.34-2.22 (m, 8H), 2.13-2.07 (m, 16H), 2.05-1.96 (m, 20H), 1.89 (s, 11H), 1.79-1.77 (m, 10H), 1.70-1.62 (m, 7H), 1.47-1.41 (m, 23H), 1.40-1.35 (m, 14H), 1.24-1.22 (m, 14H).

### Example 27

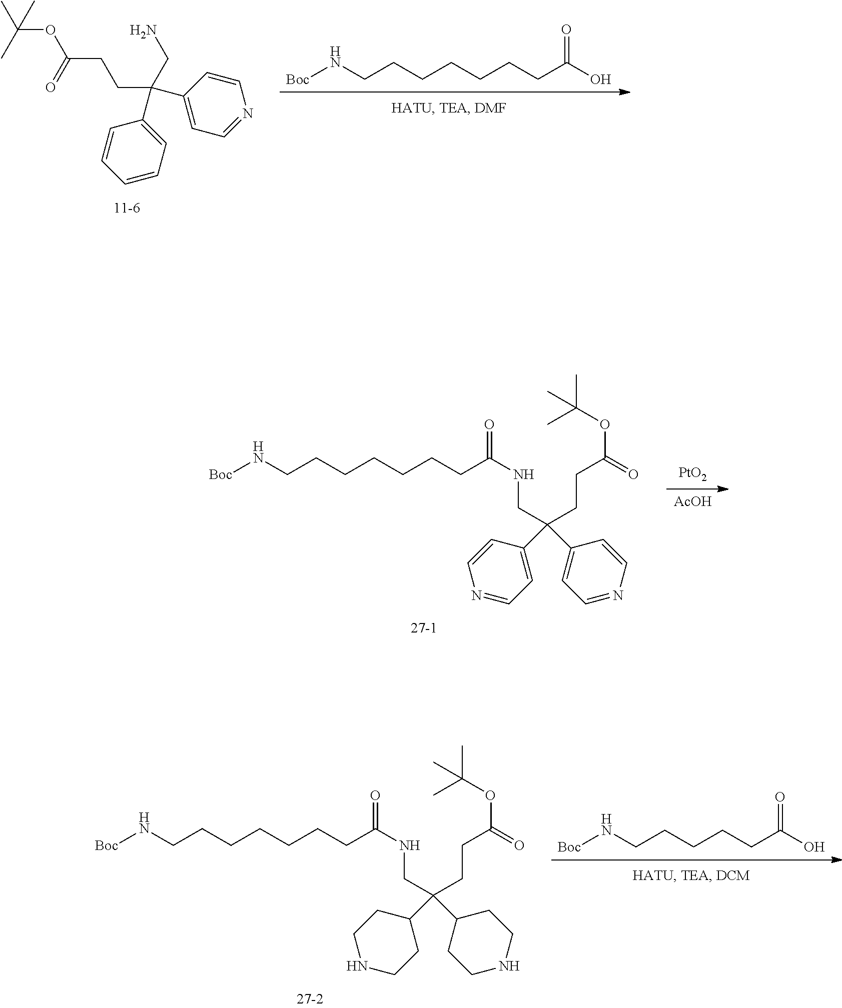

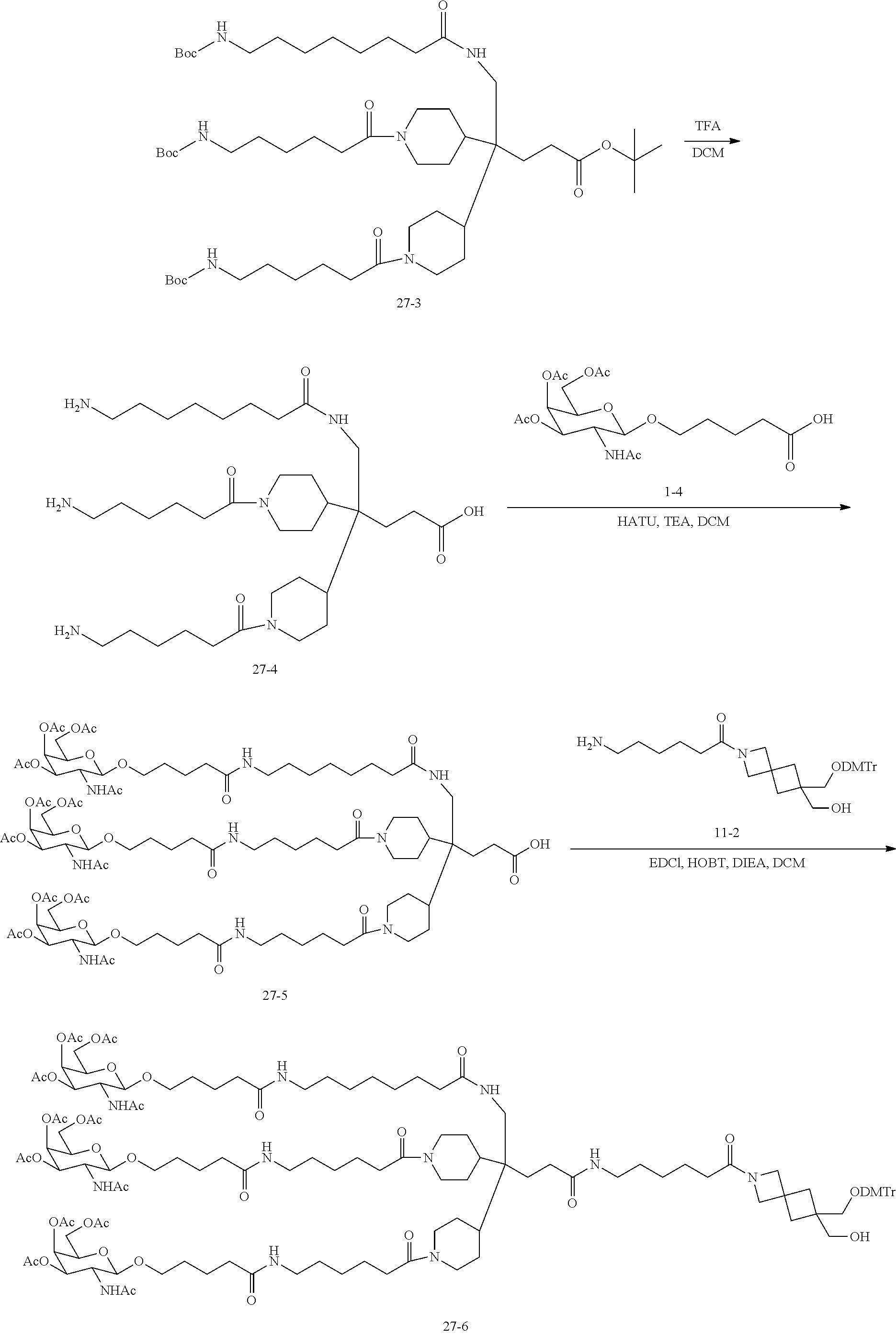

Compound 27-1: A mixture of compound 11-6 (15 g, 45.81 mmol), Boc-8-aminooctanoic acid (13.07 g, 50.39 mmol), Et₃N (6.95 g, 68.72 mmol) and HATU (19.01 g, 50.39 mmol) was stirred at rt for 1 h. Diatomite (15 g), water (300 mL) and EA (400 mL) were added to the mixture. The reaction was filtered after stirred for 30 min. The filtrate was extracted with EA (400 mL). The organic phase was washed with water (500 mL×2) and brine (300 mL), dried over Na₂SO₄, concentrated under vacuum and purified by flash column chromatography (0-10% MeOH) on silica gel to give compound 27-1 (15 g, 57% yield).

Mass calc. for C₃₂H₄₈N₄O₅: 568.3; found: 569.4 [M+H]⁺, ESI.

¹H NMR (400 MHZ, DMSO-d₆) δ 8.49-8.48 (m, 4H), 7.46-6.77 (m, 6H), 3.89 (t, J=6.4 Hz, 2H), 2.91-2.86 (m, 2H), 2.33-2.29 (m, 2H), 1.98-1.92 (m, 4H), 1.38 (s, 22H), 1.32-1.24 (m, 6H).

### Compound 27-2:

To compound 27-1 (14.5 g, 25.49 mmol) in AcOH (120 mL), PtO₂ (8 g, 35.23 mmol) was added. The reaction was stirred under 0.4 MPa hydrogen atmosphere for 88 h, then the clean liquid of the reaction mixture was poured out and concentrated under vacuum. Its pH was adjusted to 7-8 by saturated NaHCO₃. The aqueous phase was concentrated under vacuum. THF (300 mL) was added to the residue and stirred 30 min. The mixture was filtered and concentrated under vacuum to give crude compound 27-2 (15.32 g). Mass calc. for C₃₂H₆₀N₄O₅: 580.5; found: 581.6 [M+H]⁺, ESI.

### Compound 27-3:

A mixture of Boc-6-aminohexanoic acid (12.80 g, 55.38 mmol), HATU (23.87 g, 63.29 mmol) and TEA (8.0 g, 79.11 mmol) in DCM (60 mL) was stirred at rt for 1 h. Compound 27-2 (15.32 g, 26.37 mmol) in DCM (60 mL) was added to the reaction mixture and stirred at rt for 1.5 h. The reaction was quenched with water (200 mL), extracted with DCM (200 mL×3). The combined organic phase was washed with brine (200 mL), dried over Na₂SO₄, filtrated, concentrated under vacuum and purified by flash column chromatography (0-10% MeOH) on silica gel to give compound 27-3 (23 g, 75% yield).

Mass calc. for C₅₄H₉₈N₆O₁₁: 1006.7; found: 907.7 [M+H-Boc]⁺, ESI.

¹H NMR (400 MHZ, DMSO-d₆) δ 7.44-6.76 (m, 4H), 4.46 (d, J=11.6 Hz, 2H), 3.89 (d, J=12.0 Hz, 2H), 3.06-2.86 (m, 10H), 2.40-2.23 (m, 12H), 1.70-1.16 (m, 68H).

### Compound 27-4:

Compound 27-3 (10 g, 9.93 mmol) was dissolved in DCM (54 mL), then TFA (59 mL) was added to the mixture. The reaction was stirred at 30° C. for 3 h. The reaction was concentrated under vacuum without further purification to give compound 27-4 (6.64 g).

Mass calc. for C₃₅H₆₆N₆O₅: 650.5; found: 651.6 [M+H]⁺, ESI.

### Compound 27-5:

A mixture of compound 1-4 (13.32 g, 29.77 mmol), HATU (13.10 g, 34.73 mmol) and TEA (5.02 g, 49.62 mmol, 6.92 mL) in DCM (50 mL) was stirred at rt for 2 h. Compound 27-4 was added to the mixture. And the reaction was stirred at rt for another 4 h. The reaction was concentrated under vacuum and purified by prep. HPLC to give compound 27-5 (13 g, 67% yield).

Mass calc. for C₉₂H₁₄₇N₉O₃₅: 1938.0; found: 970.1 [M+2H]²⁺, ESI.

¹H NMR (400 MHZ, DMSO-d₆) δ 12.01 (s, 1H), 7.86-7.50 (m, 7H), 5.22-4.96 (m, 6H), 4.50-4.48 (m, 4H), 4.03-3.69 (m, 17H), 3.41-3.32 (m, 6H), 3.01-2.60 (m, 8H), 2.46-2.21 (m, 4H), 2.15-1.78 (m, 42H), 1.68-1.23 (m, 46H), 0.95 (t, J=7.6 Hz, 6H).

### Compound 27-6:

A mixture of compound 27-5 (2.75 g, 1.42 mmol), DIEA (383.61 mg, 2.97 mmol, 517.00 μL), EDCI (426.76 mg, 2.23 mmol) and HOBT (200.53 mg, 1.48 mmol) in DCM (30 mL) was stirred at rt for 30 min. Compound 11-2 (0.85 g, 1.48 mmol), was added to the reaction mixture. The reaction was stirred at rt for 2 h. The reaction was quenched with water (50 mL), extracted with DCM (50 mL×2). The combined organic phase was concentrated under vacuum and purified by prep. HPLC to give compound 27-6 (1.2 g, 32% yield).

Mass calc. for C₁₂₇H₁₈₉O₃₉N₁₁: 2493.9; found: 1270.0 [M+2Na]²⁺, ESI.

¹H NMR (400 MHZ, DMSO-d₆) δ 7.84-7.72 (m, 7H), 7.44-7.23 (m, 10H), 6.92-6.88 (m, 4H), 5.22 (d, J=3.6 Hz, 3H), 4.99-4.95 (m, 3H), 4.66-4.45 (m, 6H), 4.03-3.69 (m, 26H), 3.41-3.36 (m, 6H), 3.01-2.86 (m, 14H), 2.46-2.21 (m, 6H), 2.15-1.78 (m, 52H), 1.68-1.23 (m, 52H).

### Example 28

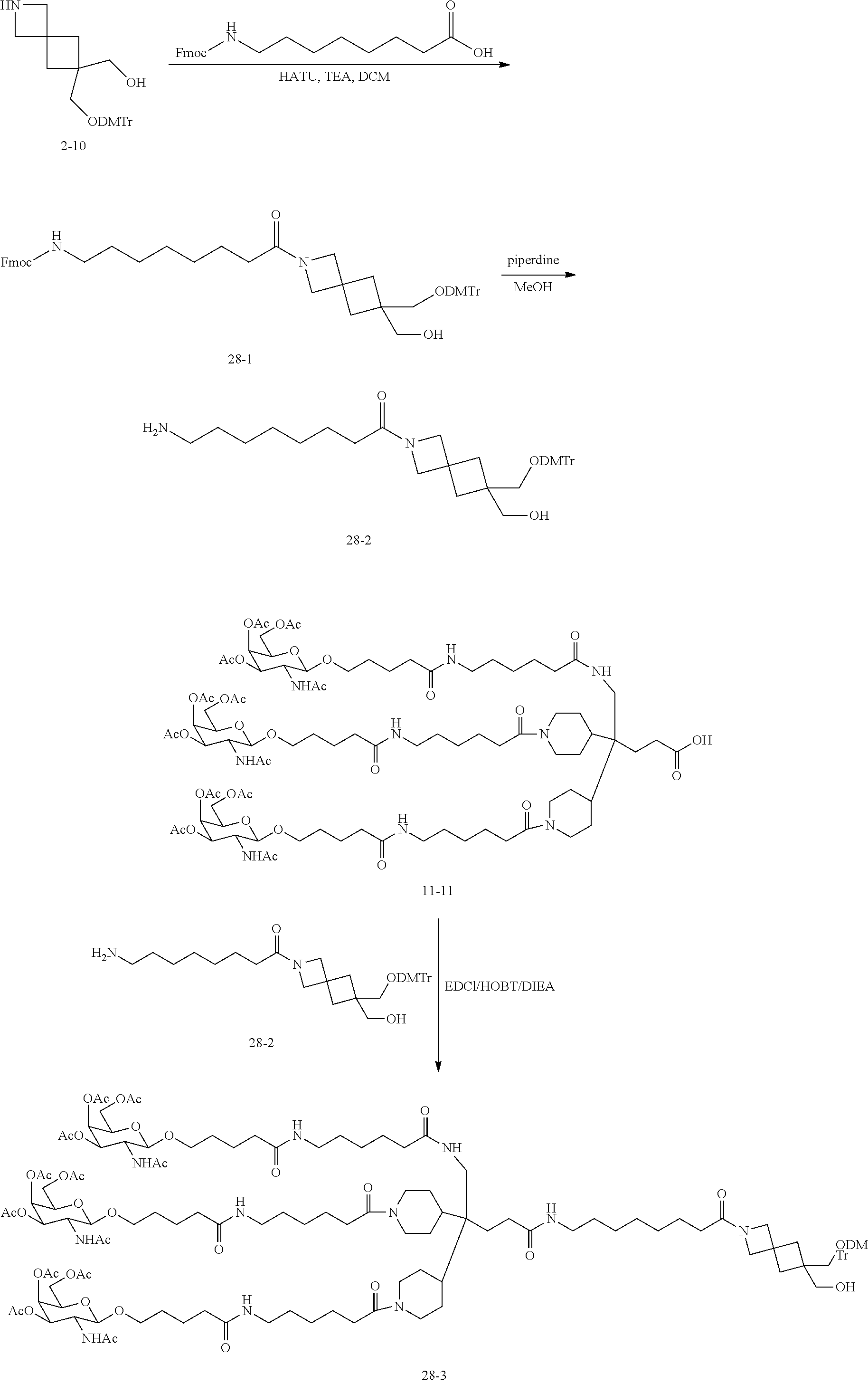

### Compound 28-1:

A mixture of 8-(Fmoc-amino) octanoic acid (1.27 g, 3.59 mmol), TEA (495.41 mg, 4.90 mmol) and HATU (1.35 g, 3.59 mmol) in DCM (15 mL) was stirred for 20 min, then compound 2-10 (1.5 g, 3.26 mmol) was added to the reaction mixture and stirring was continued for 4 h. The mixture was partitioned between DCM (50 mL) and water (35 mL), and the DCM extract was washed with brine (100 mL), dried over sodium sulfate, concentrated in vacuo and purified by flash column chromatography (DCM/MeOH=0-6%) on Al₂O₃ to give compound 28-1 as a white foam solid (600 mg, 23% yield).

¹H NMR (400 MHZ, DMSO-d₆) δ 7.87 (d, J=7.6 Hz, 2H), 7.68 (d, J=7.4 Hz, 2H), 7.44-7.35 (m, 4H), 7.34-7.28 (m, 4H), 7.27-7.17 (m, 6H), 6.89 (d, J=7.6 Hz, 4H), 4.64 (td, J=5.0, 1.6 Hz, 1H), 4.28 (d, J=6.9 Hz, 2H), 4.20 (t, J=6.8 Hz, 1H), 3.96 (s, 1H), 3.73 (d, J=3.8 Hz, 6H), 3.67 (d, J=10.4 Hz, 2H), 3.50 (s, 1H), 3.39 (t, J=5.9 Hz, 2H), 2.96 (t, J=6.6 Hz, 2H), 2.91 (s, 2H), 2.06-1.74 (m, 6H), 1.47-1.31 (m, 4H), 1.26-1.13 (m, 6H).

### Compound 28-2:

Piperidine (0.4 mL) was added to a solution of compound 28-1 (600 mg, 0.75 mmol) in MeOH (4 mL). After stirred overnight, the mixture was concentrated in vacuo to give crude compound 28-2 as a white solid (600 mg), which was used without further purification.

Mass calc. for C₃₅H₄₄N₂O₅: 600.4; found: 601.4 [M+H]⁺, ESI.

### Compound 28-3:

Compound 11-11 (1.75 g, 915.47 μmol) was dissolved in DCM (30 mL). DIEA (355 mg, 2.75 mmol), EDCI (351 mg, 1.83 mmol) and HOBT (247 mg, 1.83 mmol) were added to the mixture at 0° C. The reaction was stirred at 0° C. for 30 min. Compound 28-2 (550 mg, 915.47 μmol) was added to the reaction mixture. The reaction mixture was stirred at rt for 2 h. The reaction mixture was quenched with saturated NaHCO₃ solution (40 mL), extracted with DCM (30 mL×2), washed with brine (30 mL), dried over Na₂SO₄, concentrated and purified by prep-HPLC (C18 column, ACN/water) to afford compound 28-3 as a white foam solid (1.1 g, 48% yield).

¹H NMR (400 MHZ, DMSO-d₆) δ 7.86-7.76 (m, 4H), 7.73 (t, J=5.4 Hz, 3H), 7.46 (s, 1H), 7.40-7.35 (m, 2H), 7.31 (t, J=7.6 Hz, 2H), 7.27-7.18 (m, 5H), 6.93-6.85 (m, 4H), 5.21 (d, J=3.4 Hz, 3H), 4.95 (dd, J=11.2, 3.4 Hz, 3H), 4.65 (t, J=5.1 Hz, 1H), 4.50-4.40 (m, 5H), 4.10-3.94 (m, 10H), 3.93-3.80 (m, 5H), 3.70-3.64 (m, 11H), 3.50 (s, 1H), 3.45-3.36 (m, 5H), 3.08-3.75 (m, 14H), 2.39-2.20 (m, 6H), 2.15-1.92 (m, 31H), 1.92-1.73 (m, 21H), 1.67-1.13 (m, 52H).

### Example 29

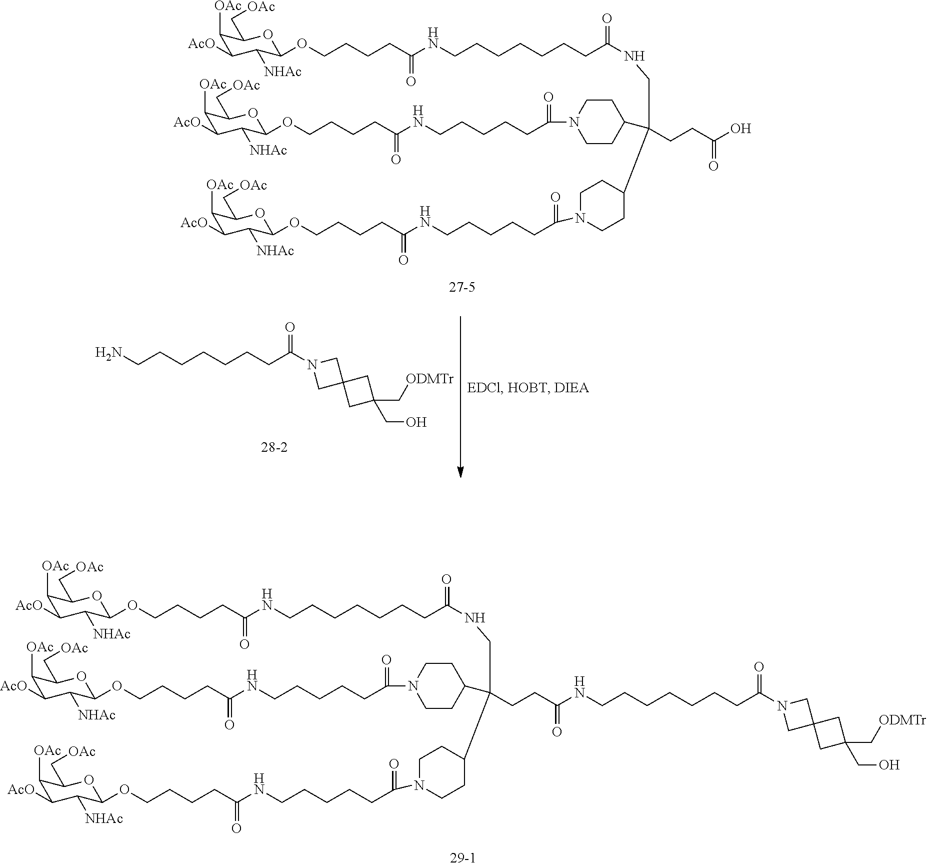

### Compound 29-1:

A mixture of compound 27-5 (2.74 g, 1.41 mmol), DIEA (365.70 mg, 2.83 mmol, 492.86 μL), EDCI (406.83 mg, 2.12 mmol) and HOBT (191.17 mg, 1.41 mmol) in DCM (30 mL) was stirred at rt for 30 min. Compound 28-2 (0.85 g, 1.41 mmol) was added to the mixture. The reaction was continued to stir at rt for 2 h. The reaction was quenched with water (50 mL), extracted with DCM (50 mL×2). The combined organic phase was concentrated under vacuum and purified by prep. HPLC to give compound 29-1 (1.1 g, 29% yield).

Mass calc. for C₁₂₉H₁₉₃O₃₉N₁₁: 2520.4; found: 1284.0 [M+2Na]²⁺, ESI.

¹H NMR (400 MHZ, DMSO-d₆) δ: 7.84-7.72 (m, 7H), 7.44-7.23 (m, 10H), 6.92-6.88 (m, 4H), 5.22 (d, J=3.6 Hz, 3H), 4.99-4.95 (m, 3H), 4.66-4.45 (m, 6H), 4.03-3.69 (m, 26H), 3.41-3.36 (m, 6H), 3.01-2.86 (m, 14H), 2.46-2.21 (m, 6H), 2.15-1.78 (m, 52H), 1.68-1.23 (m, 56H).

### Example 30

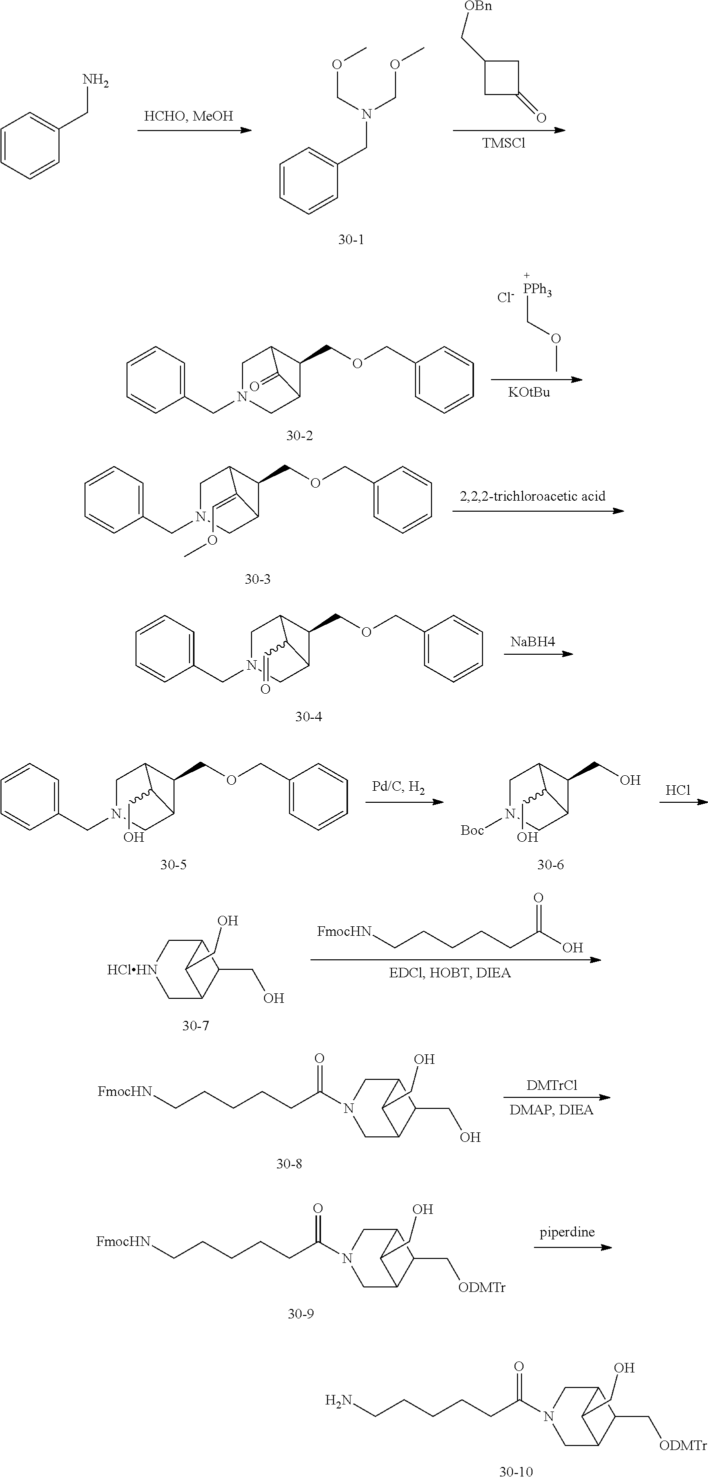

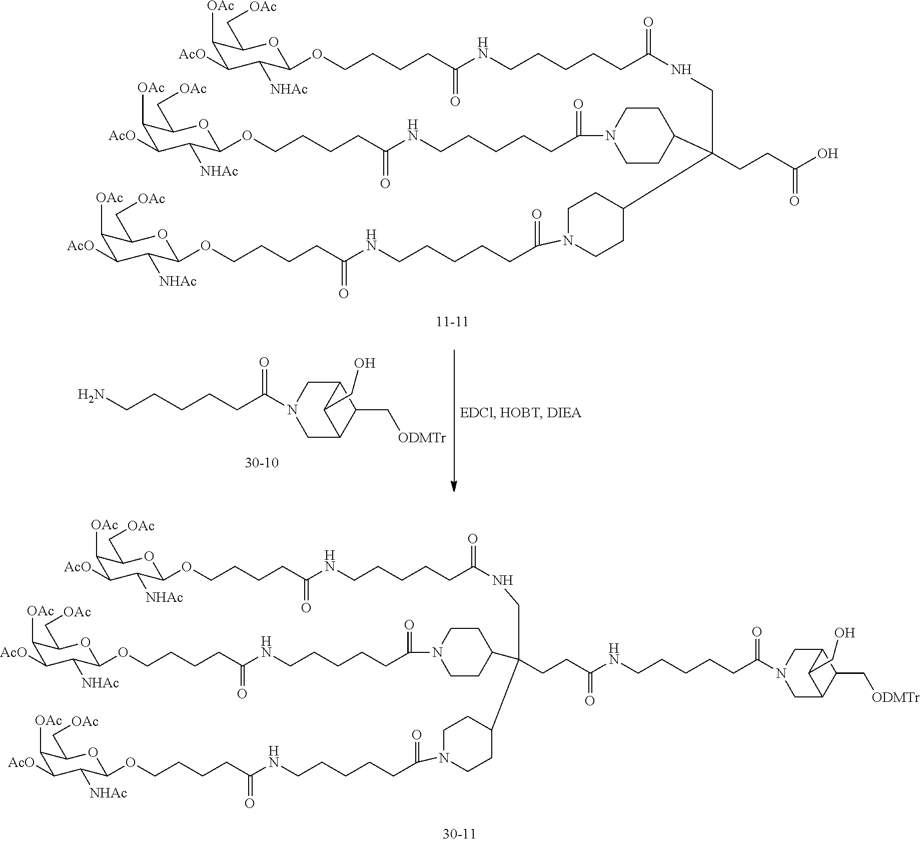

### Compound 30-1:

A mixture of benzylamine (25 g, 233.31 mmol), polyformaldehyde (42.03 g, 1.40 mol) and K₂CO₃ (32.24 g, 233.31 mmol) in MeOH (30 mL) was stirred at rt overnight. The reaction mixture was filtered and concentrated under vacuum. To the residue was added PE:EA=1:1 (100 mL), then the mixture was filtered and concentrated under vacuum to give crude compound 30-1 as a colorless oil (45.56 g).

¹H NMR (400 MHZ, DMSO-d₆) δ: 7.33-7.27 (m, 5H), 4.27 (s, 4H), 4.04 (s, 2H), 3.30 (s, 6H).

### Compound 30-2:

A mixture of 3-((Benzyloxy)methyl)cyclobutanone (23.38 g, 122.92 mmol), compound 30-1 (24 g, 122.92 mmol) in MeCN (200 mL) in a dropping funnel, and chlorotrimethylsilane (26.70 g, 245.84 mmol) in MeCN (200 mL) in another dropping funnel were mixed dropwise to acetonitrile (200 mL) upon stirring at 40° C. during 3 h. The reaction was stirred at 20° C. overnight. The pH was adjusted to 7-8 by saturated NaHCO₃ and removed ACN under vacuum. The aqueous layer was extracted with EA (200 mL×3). Combined organic phase was concentrated under vacuum. The residue was purified by C18 reversed-phase column and flash column chromatography to give 30-2 (1.5 g, 4% yield).

Mass calc. for C₂₁H₂₃O₂N: 321.2; found: 322.1 [M+H]⁺, ESI.

¹H NMR (400 MHZ, DMSO-d₆) δ 7.35-7.24 (m, 10H), 4.48 (s, 2H), 3.61 (s, 2H), 3.40 (d, J=6.8 Hz, 2H), 3.33-3.08 (m, 4H), 2.88 (d, J=2.0 Hz, 2H), 2.24 (t, J=6.8 Hz, 1H).

### Compound 30-3:

Methoxymethyl(triphenyl)phosphonium chloride (2.88 g, 8.40 mmol) was dissolved in THF (30 mL). KOtBu (1.05 g, 9.33 mmol) was added to the mixture at 0° C. The reaction mixture was stirred at 0° C. for another 30 min. Compound 30-2 (1.5 g, 4.67 mmol) was added to the mixture. The mixture was warmed to rt and stirred at rt overnight. The reaction mixture was quenched with saturated citric acid and PH was adjusted to 6-7. The reaction mixture was extracted with EA (50 mL×3), washed with brine (20 mL), dried over Na₂SO₄, concentrated under vacuum and purified by flash column chromatography (10-30% MeOH in DCM) on silica gel to give compound 30-3 as an oil (1.5 g, 92% yield).

Mass calc. for C₂₃H₂₇O₂N: 349.20; found: 350.2 [M+H]⁺, ESI.

¹H NMR (400 MHZ, DMSO-d₆) δ 7.37-7.26 (m, 10H), 6.02 (s, 1H), 4.47 (s, 2H), 3.85 (s, 2H), 3.54 (d, J=7.6 Hz, 2H), 3.46 (s, 3H), 3.13-3.09 (m, 4H), 2.88 (d, J=2 Hz, 2H), 2.24 (t, J=6.8 Hz, 1H).

### Compound 30-4:

2,2,2-trichloroacetic acid (883.64 mg, 5.41 mmol) in water (15 mL) was added to compound 30-3 (1.5 g, 4.29 mmol) in ACN (45 mL). The reaction was stirred at rt overnight. The reaction was quenched with saturated NaHCO₃ and pH was adjusted to 7-8. The reaction mixture was extracted with EA (20 mL×3), washed with brine (10 mL), dried over Na₂SO₄, concentrated under vacuum and purified by flash column chromatography (10-30% EA in PE) on silica gel to give compound 30-4 as an oil (600 mg, 42% yield).

Mass calc. for C₂₂H₂₅O₂N: 335.25; found: 336.2 [M+H]⁺, ESI.

¹H NMR (400 MHZ, DMSO-d₆) δ 9.90 (s, 1H), 7.37-7.26 (m, 10H), 4.41 (s, 2H), 3.85 (s, 2H), 3.44 (d, J=7.6 Hz, 2H), 3.03-2.89 (m, 4H), 2.86 (d, J=4 Hz, 1H), 2.69 (s, 2H), 2.04 (t, J=6.8 Hz, 1H).

### Compound 30-5:

Compound 30-4 (600 mg, 1.79 mmol) was dissolved in MeOH (18 mL), then NaBH₄ (148.88 mg, 3.94 mmol) was added slowly in several portions. The reaction was stirred at rt for 1 h. The reaction was quenched with water. pH was adjusted to 6-7 with saturated citric acid. The reaction was concentrated under vacuum to remove MeOH. The residue was extracted with DCM (20 mL×3), washed with brine (10 mL), dried over Na₂SO₄, concentrated under vacuum to give compound 30-5 (600 mg).

Mass calc. for C₂₂H₂₇NO₂: 337.15; found: 338.2 [M+H]⁺, ESI.

¹H NMR (400 MHZ, DMSO-d₆) δ 7.37-7.26 (m, 10H), 4.47 (s, 2H), 3.90 (s, 2H), 3.69 (d, J=7.6 Hz, 2H), 3.62-3.59 (m, 4H), 3.11 (d, J=10.8 Hz, 2H), 2.61-2.56 (m, 2H), 2.19-2.04 (m, 2H).

### Compound 30-6:

A mixture of compound 30-5 (200 mg, 592.67 μmol), Pd/C (100.00 mg, 823.38 μmol) in MeOH (6 mL) was stirred under 0.4 MPa H2 for 88 hr. The mixture was filtered and concentrated under vacuum, purified by prep. HPLC to give compound 30-6 (70 mg, 46% yield).

Mass calc. for C₁₃H₂₃NO₂: 257.2; found: 158.2 [M−Boc+H]⁺, ESI.

¹H NMR (400 MHZ, CDCl₃) δ 3.96 (d, J=8.0 Hz, 4H), 3.70-3.68 (m, 4H), 3.25-3.17 (m, 4H), 2.36-2.30 (m, 2H), 2.00-1.95 (m, 2H), 1.49 (s, 9H).

### Compound 30-7:

Compound 30-6 (1 g, 3.89 mmol) was dissolved in HCl/1,4-Dioxane (4M, 10 mL). The reaction mixture was stirred at rt for 3 h. The reaction mixture was concentrated under vacuum to give crude product 30-7 (610 mg), which was used in the next step without further purification.

Mass calc. for C₈H₁₅NO₂: 157.2; found: 158.1 [M+H]⁺, ESI.

### Compound 30-8:

A mixture of DIEA (1.25 g, 9.70 mmol, 1.69 mL), HOBT (629.15 mg, 4.66 mmol), EDCI (892.59 mg, 4.66 mmol), Fmoc-6-aminohexanoic acid (1.37 g, 3.88 mmol) in DCM (30 mL) was stirred at 0° C. for 30 min. Compound 30-7 (610 mg, 3.88 mmol) was added to the reaction mixture and stirred at rt overnight. The reaction was quenched with water, washed with 10% citric acid (10 mL) and saturated NaHCO₃ solution (10 mL) and brine (10 mL), dried over Na₂SO₄, concentrated under vacuum and purified by flash column chromatography (0-10% MeOH) on silica gel to give compound 30-8 (1.55 g, 81% yield).

Mass calc. for C₂₉H₃₆N₂O₅: 492.3; found: 493.3 [M+H]⁺, ESI.

¹H NMR (400 MHZ, DMSO-d₆) δ 7.90-7.68 (m, 4H), 7.44-7.25 (m, 5H), 4.56 (t, J=5.2 Hz, 2H), 4.31-4.20 (m, 3H), 3.70-3.67 (m, 4H), 3.55 (s, 2H), 3.31 (s, 4H), 2.99-2.95 (m, 2H), 2.29-2.21 (m, 2H), 1.71 (t, J=6.0 Hz, 2H), 1.53-1.24 (m, 6H).

### Compound 30-9:

Compound 30-8 (1.55 g, 3.15 mmol), DMAP (38.44 mg, 314.65 μmol), DIEA (610.00 mg, 4.72 mmol, 822.10 μL) was dissolved in DCM (50 mL). DMTrCl (1.17 g, 3.46 mmol) was added to the reaction mixture at 0° C. in portions. The reaction was stirred at rt overnight. The reaction mixture was concentrated under vacuum and purified by flash column chromatography (20-50% EA in PE) on Al₂O₃ to give compound 30-9 (1 g, 40% yield).

Mass calc. for C₅₀H₅₄N₂O₇: 794.4; found: 817.4 [M+Na]⁺, ESI.

¹H NMR (400 MHZ, DMSO-d₆) δ 7.90-7.68 (m, 4H), 7.44-7.25 (m, 14H), 6.90 (d, J=8.8 Hz, 4H), 4.56 (t, J=5.2 Hz, 1H), 4.31-4.20 (m, 3H), 3.74 (s, 6H), 3.70-3.67 (m, 4H), 3.55 (s, 2H), 3.31 (s, 4H), 2.99-2.95 (m, 2H), 2.29-2.21 (m, 2H), 1.89-1.67 (m, 2H), 1.53-1.24 (m, 6H).

### Compound 30-10:

Compound 30-9 (700 mg, 880.54 μmol) was dissolved in MeOH (14 mL), then piperidine (1.4 mL) was added to the mixture. The reaction mixture was stirred at rt overnight. The reaction mixture concentrated under vacuum and purified by flash column chromatography (0-10% MeOH) on Al₂O₃ to give compound 30-10 as a white foam solid (300 mg, 59% yield).

Mass calc. for C₃₅H₄₄N₂O₅: 572.3; found: 595.3 [M+Na]⁺, ESI.

### Compound 30-11:

A mixture of compound 11-11 (1.00 g, 523.80 μmol), DIEA (169.25 mg, 1.31 mmol, 228.09 μL), HOBT (84.93 mg, 628.57 μmol) and EDCI (120.50 mg, 628.57 μmol) in DCM (50 mL) was stirred at 0° C. for 30 min. Compound 30-10 (300 mg, 523.80 μmol) was added to the mixture. And the reaction was stirred at rt overnight. The reaction mixture was quenched with water (20 mL), extracted with DCM (25 mL×2), washed with brine (50 mL), dried over Na₂SO₄, concentrated under vacuum and purified by HPLC to give compound 30-11 (595 mg, 46% yield).

Mass calc. for C₁₂₅H₁₈₅N₁₁O₃₉: 2464.3; found: 1255.2 [M+2Na]²⁺, ESI.

¹H NMR (400 MHZ, DMSO-d₆) δ 7.83-7.69 (m, 7H), 7.43 (s, 1H), 7.37-7.20 (m, 9H), 6.91-6.89 (m, J=8.8 Hz, 4H), 5.22 (d, J=3.2 Hz, 3H), 4.99-4.95 (m, 3H), 4.49-4.46 (m, 6H), 4.03 (s, 9H), 3.91-3.85 (m, 5H), 3.74 (s, 6H), 3.74-3.26 (m, 9H), 3.01-2.99 (m, 10H), 2.90-2.87 (m, 2H), 2.27-2.20 (m, 9H), 2.12 (s, 11H), 2.06-2.02 (m, 8H), 2.02 (s, 11H), 1.89 (s, 11H), 1.78 (s, 9H), 1.62-1.21 (m, 52H).

### Example 31

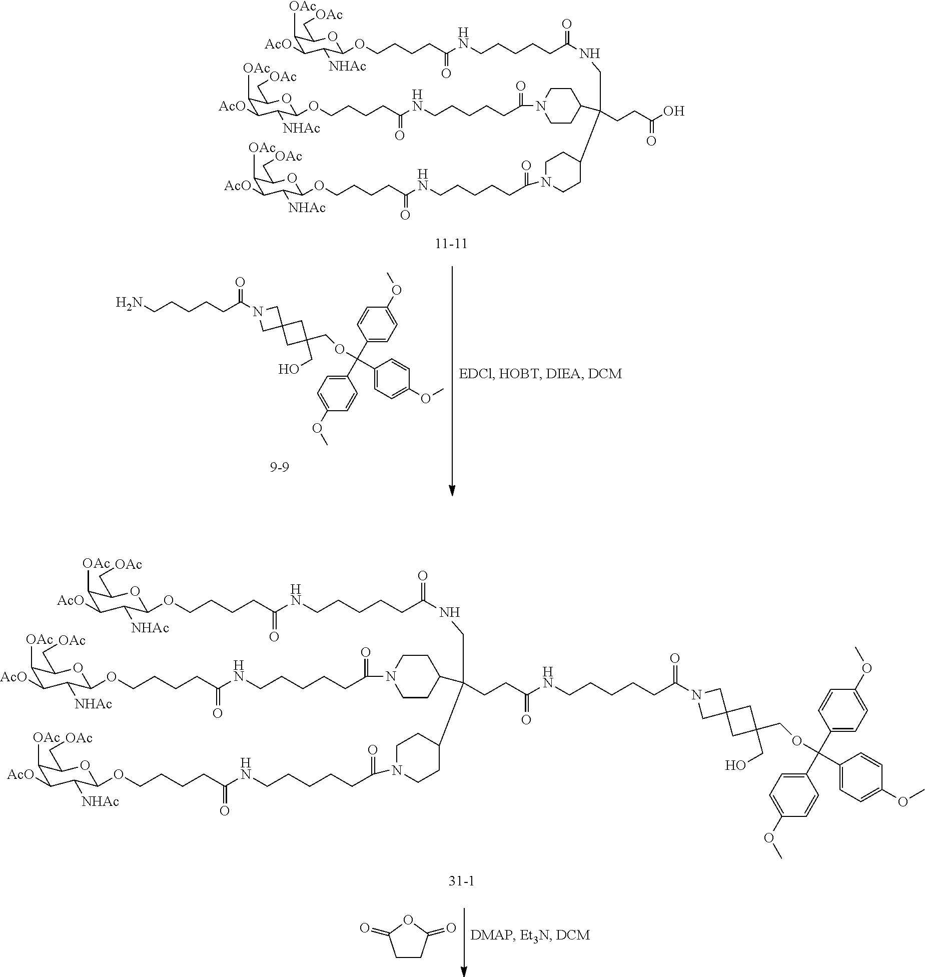

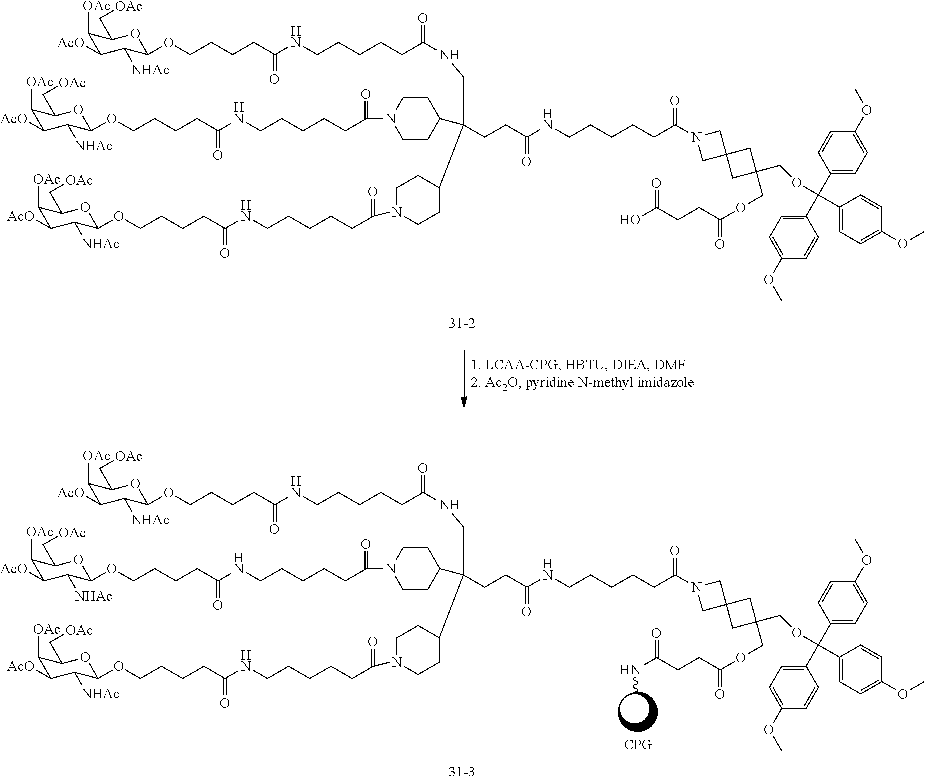

### Compound 31-1:

A mixture of compound 11-11 (3.17 g, 1.66 mmol), HOBT (448.34 mg, 3.32 mmol), EDCI (954.11 mg, 4.98 mmol) and DIEA (857.66 mg, 6.64 mmol) in DCM (20 mL) was stirred for 15 min, then compound 9-9 (1.00 g, 1.66 mmol) was added to the reaction mixture and stirring was continued for 4 h. The mixture was partitioned between DCM (50 mL) and water (35 mL), and the DCM extract was washed with brine (100 ml), dried over sodium sulfate and concentrated in vacuo. The residue was purified by reverse phase column (C18 column) to provide compound 31-1 (2.25 g, 54% yield).

¹H NMR (400 MHZ, DMSO-d₆) δ 7.85-7.78 (m, 4H), 7.72 (t, J=5.4 Hz, 3H), 7.45 (s, 1H), 7.27-7.20 (m, 6H), 6.91-6.85 (m, 6H), 5.21 (d, J=3.4 Hz, 3H), 4.99-4.94 (m, 3H), 4.64 (t, J=5.1 Hz, 1H), 4.50-4.41 (m, 5H), 4.05-3.95 (m, 10H), 3.91-3.83 (m, 5H), 3.75-3.65 (m, 14H), 3.51 (s, 1H), 3.44-3.31 (m, 5H), 3.06-2.94 (m, 10H), 2.90 (s, 2H), 2.89-2.78 (m, 2H), 2.41-2.30 (m, 2H), 2.28-2.21 (m, 4H), 2.10 (s, 9H), 2.08-2.00 (m, 10H), 1.99 (s, 11H), 1.96-1.92 (m, 2H), 1.89 (s, 9H), 1.86-1.80 (m, 2H), 1.77 (s, 9H), 1.66-1.31 (m, 35H), 1.30-1.13 (m, 13H).

### Compound 31-2:

To a solution of compound 31-1 (150 mg, 0.06 mmol) in anhydrous DCM (1.0 mL), DMAP (14 mg, 0.12 mmol) and TEA (24 mg, 0.24 mmol) were added, followed by adding succinic anhydride (20 mg, 0.2 mmol). The reaction mixture was stirred at rt for 3 h, LCMS showed the starting material was consumed completely. The reaction mixture was diluted with DCM (10 mL) and washed with H₂O (3 mL×4), then washed with brine (3 mL×4), the organic layer was concentrated to give compound 31-2 as a white solid (130 mg, 97% yield).

Mass calc. for C₁₃₀H₁₉₁N₁₁O₄₃: 2594.31; found: 1297.4 [M+2H]²⁺, ESI.

¹HNMR (600 MHZ, DMSO-d₆) δ 7.92-7.69 (m, 7H), 7.50-7.41 (m, 1H), 7.31-7.12 (m, 9H), 6.92-6.89 (m, 6H), 5.21 (d, J=6.0 Hz, 3H), 4.98-4.95 (m, 3H), 4.52-4.41 (m, 5H), 4.07-4.01 (m, 12H), 3.90-3.83 (m, 5H), 3.80-3.64 (m, 14H), 3.49-3.25 (m, 17H), 3.05-2.84 (m, 14H), 2.47-2.23 (m, 8H), 2.12-1.77 (m, 52H) 1.61-1.21 (m, 46H).

### Solid Support 31-3:

Native amino-LCAA-CPG (loading value: 75 umol/g, 1000 Å) was washed with ACN (100 mL×2), DMF (100 mL×2) and DCM (100 mL×2), then dried under high vacuum overnight.

To a solution of succinate 31-2 (130 mg, 0.05 mmol) and HBTU (53 mg, 0.14 mmol) in anhydrous DMF (1.5 mL), DIPEA (30 mg, 0.23 mmol) was added, the reaction mixture was shaken at rt for 10 min, then native amino-LCAA-CPG (300 mg, loading 75 umol/g) was added, the suspension was shaken at rt for 20 h, then filtered, washed with DMF (20 mL×5), ACN (20 mL×5), DCM (20 mL×5) until TLC showed no spot at 254 nm in eluent. The solid support was dried under vacuum for 2 h to give solid support (300 mg). The unreacted amines on the solid support were capped by stirring with Ac2O/pyridine/N-methyl imidazole (90 μL/1.0 mL/80 μL) at RT for 1 h, washed with DMF (20 mL×5), CAN (20 mL×5), DCM (20 mL×5) until TLC showed no spot at 254 nm in eluent. The solid support was dried under vacuum for 15 h to give solid support 31-3 (300 mg). For loading calculation, take 5.3 mg of the dried loaded CPG and add 20 mL of 3% DCA on DCM. Shake the solution and measure UV absorbance at 482 nm. Make sure the absorbance value is below 1.0 units to ensure no signal saturation. Then apply the following equation:

Loading in umol/g=(total volume of DCA added in mL)*(Abs value at 482 nm)*1000)/78.3*(mg of CPG taken)
Total volume of DCA added in mL=20 mL
Abs value at 482 nm=0.681
Mg of CPG taken=5.3 mg
Loading in umol/g=((20)*(0.681)*1000)/78.3*(5.3)=32.82 umol/g.

### Example 32

Synthesis of GalNAc-Conjugated and Unconjugated Oligonucleotide and siRNAs

### GalNAc-Conjugated Phosphoramidites:

The synthesis of all other GalNAc-conjugated phosphoramidites was following the procedures described in examples 2-7, 9 and 11.

### GalNAc-Conjugated Succinate and Loading to Solid Support:

The synthesis of all other GalNAc-conjugated succinate and loading to solid support was following the procedures described in examples 2, 4, 9-11 and 31.

siRNA Sequence and Chemical Modification for Targeting Human Proprotein Convertase Subtilisin/Kexin Type 9 (hPCSK9):

Sense strand (5′ to 3′):
(1)
CmsUmsAmGmAmCmCfUmGfUmdTUmUmGmCmUmUmUmUmGmUm-
GalNAc:
(2)
GalNAc-CmUmAmGmAmCmCfUmGfUmdTUmUmGmCmUmUmUmUm
sGmsUm
(3)
GalNAc-CmUmAmGmAmCmCfUmGfUmdTUmUmGmCmUmUmUmUm
GmUm-GalNAc
Antisense strand (5′ to 3′):
AmsCfsAmAfAfAfGmCfAmAfAmAfCmAfGmGfUmCfUmAmGmsAms
Am

siRNA sequence and chemical modification for targeting mouse complement component 3 (mC3):

Sense strand (5′ to 3′):
(1)
UmsGmsGmUmCmAmAfGfGfUmCmUmUmCmUmCmUmCmUm-GalNAc
(2)
GalNAc-UmGmGmUmCmAmAfGfGfUmCmUmUmCmUmCmUmsCmsUm
Antisense strand (5′ to 3′):
(E)-VP-UmGfAmGfAmGfAmAfGmAfCmCfUmUfGmAfCmsCfsAm

siRNA Sequence and Chemical Modification for Targeting Mouse Transthyretin (mTTR):

Sense strand (5′ to 3′):
(1)
AfsAmsCfAmGfUmGfUmUfCfUfUmGfCmUfCmUfAmUfAmAf-
GalNAc
(2)
GalNAc-AfAmCfAmGfUmGfUmUfCfUfUmGfCmUfCmUfAmUfs
AmsAf
Antisense strand (5′ to 3′):
UmsUfAmUfAmGfAmGfCmAfAmGmAmAfCmAfCmUfGmUfUmsUmsUm
s: phosphorothioate, otherwise phosphodiester,
m: 2′-OMe;
f: 2′-F;
(E)-VP: (E)-vinylphosphonate.

### General Procedure for Preparation:

1. Loading: GalNAc- or nucleoside-preloaded CPG (0.19 g, 500 A, ˜10 μmol) in 5 mL syringe.
2. Washing: acetonitrile (2.0 mL, 0.3 min, repeated two times, at room temperature).
3. Detritylation: 3% trichloroacetic acid in dichloromethane (2.0 mL, 0.7 min, repeated four times, at room temperature).
4. Washing: acetonitrile (2.0 mL, 0.3 min, repeated two times, at room temperature).
5. Coupling: 0.067 M reactant monomer in acetonitrile (1.0 mL) and 0.30 M 5-(Benzylthio)-1H-tetrazole (BTT) in acetonitrile (1.0 mL) as activator (7.0 min, repeated three times, at room temperature)
6. Sulfurization or oxidation: N,N-Dimethyl-N′-(3-thioxo-3H-1,2,4-dithiazol-5-yl) methanimidamide (DDTT, 4.80 M, in pyridine/acetonitrile=2/1, 2.0 mL, 1 min, two times, at room temperature) or 0.05 M I₂ in pyridine/H₂O=80/20 (v/v) (2.0 mL, 1 min, two time, at room temperature).
7. Capping: 1-Methylimidazole (NMI)/acetonitrile=15/85 (v/v) (2.0 mL) and acetic anhydride/acetonitrile=20/80 (v/v) (0.9 mL, 1 min, one time, at room temperature).
8. Washing: acetonitrile (2.6 mL, 0.3 min, repeated two times, at room temperature).
9. The synthesis program was automatically circulated for 19 times.
10. The solid support was immersed in 10% DEA for 20 min. The solid support was suspended in NH₃·H₂O (5 mL) and stirred at 40° C. for 16 h in a 48 mL of sealed tube. The reaction mixture was allowed to cool to 25° C. Then the solid support was filtered and the aqueous phase was concentrated in vacuum to give a yellow solution.
11. 40 mL ethanol was added to the filtrate (10 mL), then followed by adding 0.3 mL NaCl (3M). The centrifuge tube was standing for 20 mins at −20° C. Then the tube was centrifuged. The supernatant liquid was discarded and the remained solid was collected.
12. The white solid was purified by prep-HPLC (column: O-C18 150*40 mm*10 um; mobile phase: [0.1 M TEAB-ACN]; B %: 14%-24%, 30 min).
13. The desired compound was obtained as a white solid after lyophilization.

### Annealing Steps:

1. Calculate the mole value of duplex (for example, 2 mg of duplex, the mole value=2 mg/duplex M.W., free acid), which is the single strand mole value.
2. Dissolve the sense strand and antisense strand in ultrapure water (DNase/RNase-free, sterile) at 25° C., respectively. The concentration of oligos was quantified by UV-vis.
3. Mix the two solutions with molar ratio of 1:1.
4. Placed the mix samples at room temperature over 10 min. Test by HPLC to monitor whether the ratio was appropriate.
5. Aliquot the solution into tubes and lyophilize to give the siRNA samples.

TABLE 1
siRNA ID and their GalNAc types and positions at sense strands

| siRNA ID | targets | GalNAc type and position at sense strand |
|---|---|---|
| siRNA-1 | hPCSK9 | GalNAc-1 at 3′ end |
| siRNA-2 | hPCSK9 | GalNAc-2 at 3′ end |
| siRNA-3 | hPCSK9 | GalNAc-3 at 3′ end |
| siRNA-4 | hPCSK9 | GalNAc-4 at 3′ and 5′ end |
| siRNA-5 | hPCSK9 | GalNAc-5 at 5′ end |
| siRNA-6 | hPCSK9 | GalNAc-6 at 3′ end |
| siRNA-7 | hPCSK9 | GalNAc-7 at 3′ end |
| siRNA-8 | hPCSK9 | GalNAc-1 at 3′ end (Cy3 at 5′ end) |
| SiRNA-9 | hPCSK9 | GalNAc-8 at 5′ end |
| siRNA-10 | hPCSK9 | GalNAc-9 at 5′ end |
| siRNA-11 | hPCSK9 | GalNAc-10 at 5′ end |
| siRNA-12 | hPCSK9 | GalNAc-11 at 5′ end |
| siRNA-13 | hPCSK9 | GalNAc-3 at 5′ end |
| siRNA-14 | hPCSK9 | GalNAc-12 at 3′ end |
| SIRNA-15 | hPCSK9 | GalNAc-13 at 3′ end |
| siRNA-16 | hPCSK9 | GalNAc-14 at 3′ end |
| siRNA-17 | hPCSK9 | GalNAc-15 at 3′ end |
| siRNA-18 | hPCSK9 | GalNAc-16 at 3′ end |
| siRNA-19 | hPCSK9 | GalNAc-17 at 3′ end |
| siRNA-20 | hPCSK9 | GalNAc-18 at 3′ end |
| siRNA-21 | hPCSK9 | GalNAc-7 at 5′ end |
| siRNA-22 | hPCSK9 | GalNAc-19 at 5′ end |
| SIRNA-23 | mC3 | GalNAc-1 at 3′ end |
| siRNA-24 | mC3 | GalNAc-6 at 3′ end |
| siRNA-25 | mC3 | GalNAc-7 at 3′ end |
| siRNA-26 | mTTR | GalNAc-1 at 3′ end |
| siRNA-27 | mTTR | GalNAc-6 at 3′ end |
| siRNA-28 | mTTR | GalNAc-7 at 3′ end |
| siRNA-29 | hPCSK9 | GalNAc-2 at 5′ end |
| siRNA-30 | hPCSK9 | GalNAc-20 at 3′ end |
| siRNA-31 | hPCSK9 | GalNAc-21 at 3′ end |
| siRNA-32 | hPCSK9 | GalNAc-22 at 3′ end |
| siRNA-33 | hPCSK9 | GalNAc-23 at 3′ end |
| siRNA-34 | hPCSK9 | GalNAc-24 at 3′ end |
| siRNA-35 | hPCSK9 | GalNAc-25 at 3′ end |
| siRNA-36 | mTTR | GalNAc-3 at 3′ end |
| siRNA-37 | mTTR | GalNAc-3 at 5′ end |
| siRNA-38 | mTTR | GalNAc-5 at 5′ end |
| siRNA-39 | mTTR | GalNAc-2 at 3′ end |
| siRNA-40 | mTTR | GalNAc-2 at 5′ end |
| siRNA-41 | hPCSK9 | GalNAc-26 at 3′ end |
| siRNA-42 | hPCSK9 | GalNAc-27 at 3′ end |
| siRNA-43 | hPCSK9 | GalNAc-28 at 3′ end |
| siRNA-44 | hPCSK9 | GalNAc-29 at 3′ end |
| siRNA-45 | hPCSK9 | GalNAc-30 at 3′ end |
| siRNA-46 | hPCSK9 | GalNAc-31 at 3′ end |

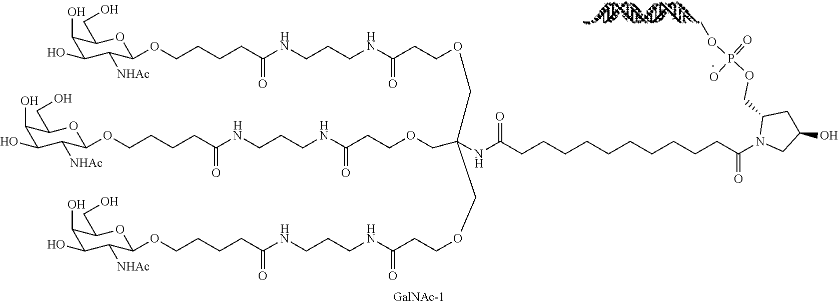 [SMILES: COP(=O)(O)OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

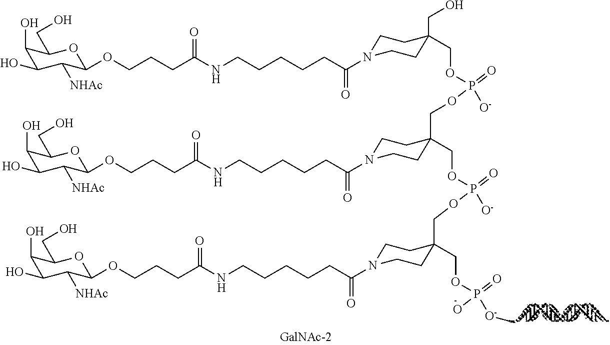

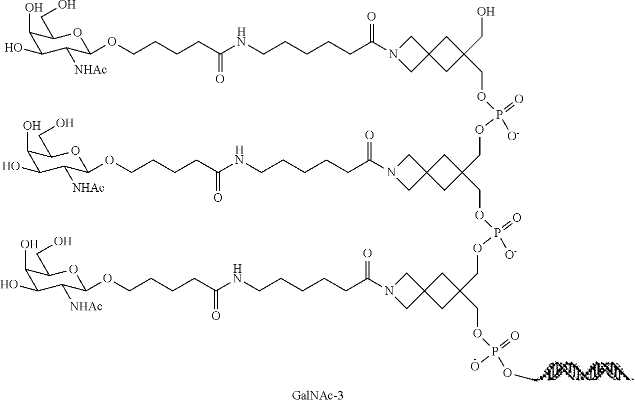 [SMILES: COP(=O)(O)OCC1(COP(=O)([O-])OCC2(COP(=O)([O-])OCC3(CO)CC4(CN(C(=O)CCCCCNC(=O)CCCCOC5OC(CO)C(O)C(O)C5NC(C)=O)C4)C3)CC3(CN(C(=O)CCCCCNC(=O)CCCCOC4OC(CO)C(O)C(O)C4NC(C)=O)C3)C2)CC2(CN(C(=O)CCCCCNC(=O)CCCCOC3OC(CO)C(O)C(O)C3NC(C)=O)C2)C1]

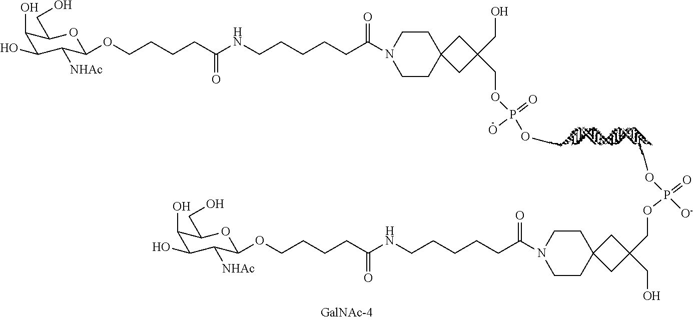 [SMILES: COP(=O)(O)OCC1(CO)CC2(CCN(C(=O)CCCCCNC(=O)CCCCOC3OC(CO)C(O)C(O)C3NC(C)=O)CC2)C1 ; COP(=O)([O-])OCC1(CO)CC2(CCN(C(=O)CCCCCNC(=O)CCCCOC3OC(CO)C(O)C(O)C3NC(C)=O)CC2)C1]

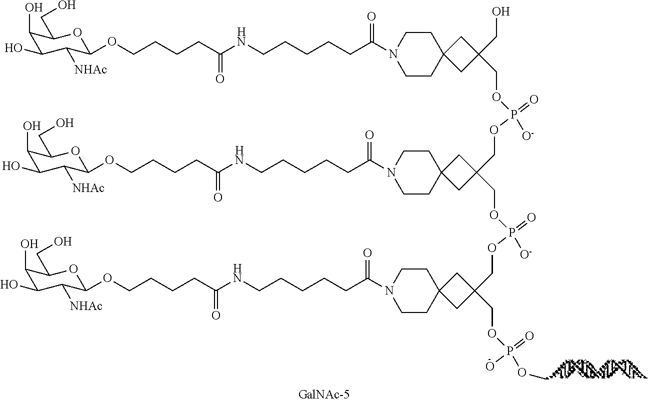 [SMILES: COP(=O)(O)OCC1(COP(=O)([O-])OCC2(COP(=O)([O-])OCC3(CO)CC4(CCN(C(=O)CCCCCNC(=O)CCCCOC5OC(CO)C(O)C(O)C5NC(C)=O)CC4)C3)CC3(CCN(C(=O)CCCCCNC(=O)CCCCOC4OC(CO)C(O)C(O)C4NC(C)=O)CC3)C2)CC2(CCN(C(=O)CCCCCNC(=O)CCCCOC3OC(CO)C(O)C(O)C3NC(C)=O)CC2)C1]

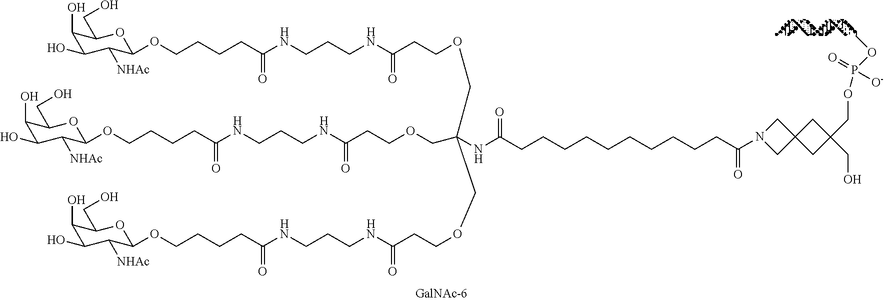 [SMILES: COP(=O)([O-])OCC1(CO)CC2(CN(C(=O)CCCCCCCCCCC(=O)NC(COCCC(=O)NCCCNC(=O)CCCCOC3OC(CO)C(O)C(O)C3NC(C)=O)(COCCC(=O)NCCCNC(=O)CCCCOC3OC(CO)C(O)C(O)C3NC(C)=O)COCCC(=O)NCCCNC(=O)CCCCOC3OC(CO)C(O)C(O)C3NC(C)=O)C2)C1]

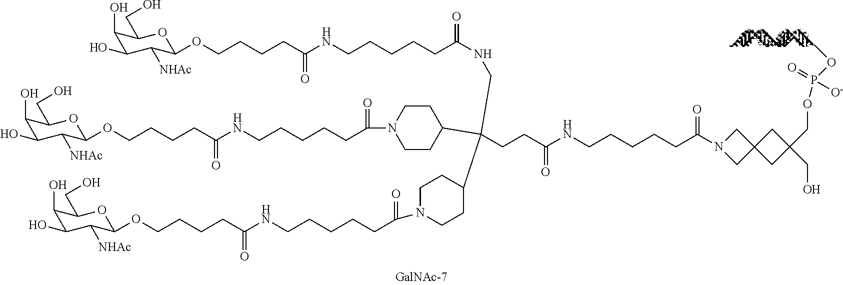 [SMILES: COP(=O)([O-])OCC1(CO)CC2(CN(C(=O)CCCCCNC(=O)CCC(CNC(=O)CCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)C2)C1]

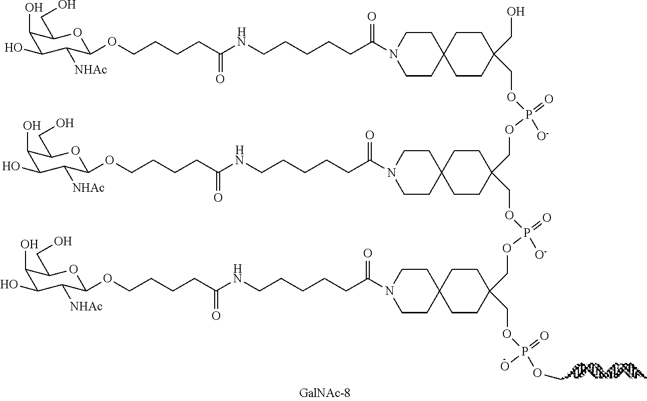 [SMILES: COP(=O)(O)OCC1(COP(=O)([O-])OCC2(COP(=O)([O-])OCC3(CO)CCC4(CCN(C(=O)CCCCCNC(=O)CCCCOC5OC(CO)C(O)C(O)C5NC(C)=O)CC4)CC3)CCC3(CCN(C(=O)CCCCCNC(=O)CCCCOC4OC(CO)C(O)C(O)C4NC(C)=O)CC3)CC2)CCC2(CCN(C(=O)CCCCCNC(=O)CCCCOC3OC(CO)C(O)C(O)C3NC(C)=O)CC2)CC1]

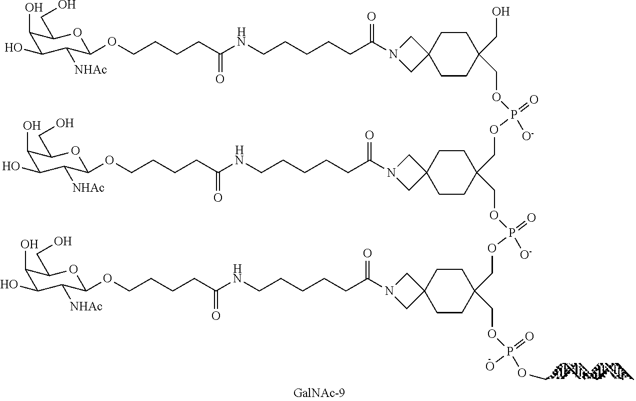 [SMILES: COP(=O)(O)OCC1(COP(=O)([O-])OCC2(COP(=O)([O-])OCC3(CO)CCC4(CC3)CN(C(=O)CCCCCNC(=O)CCCCOC3OC(CO)C(O)C(O)C3NC(C)=O)C4)CCC3(CC2)CN(C(=O)CCCCCNC(=O)CCCCOC2OC(CO)C(O)C(O)C2NC(C)=O)C3)CCC2(CC1)CN(C(=O)CCCCCNC(=O)CCCCOC1OC(CO)C(O)C(O)C1NC(C)=O)C2]

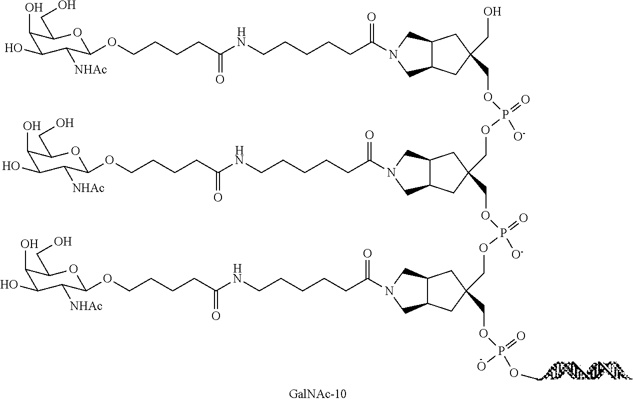 [SMILES: COP(=O)(O)OC[C@@]1(COP(=O)([O-])OC[C@@]2(COP(=O)([O-])OC[C@@]3(CO)C[C@H]4CN(C(=O)CCCCCNC(=O)CCCCOC5OC(CO)C(O)C(O)C5NC(C)=O)C[C@H]4C3)C[C@H]3CN(C(=O)CCCCCNC(=O)CCCCOC4OC(CO)C(O)C(O)C4NC(C)=O)C[C@H]3C2)C[C@H]2CN(C(=O)CCCCCNC(=O)CCCCOC3OC(CO)C(O)C(O)C3NC(C)=O)C[C@H]2C1]

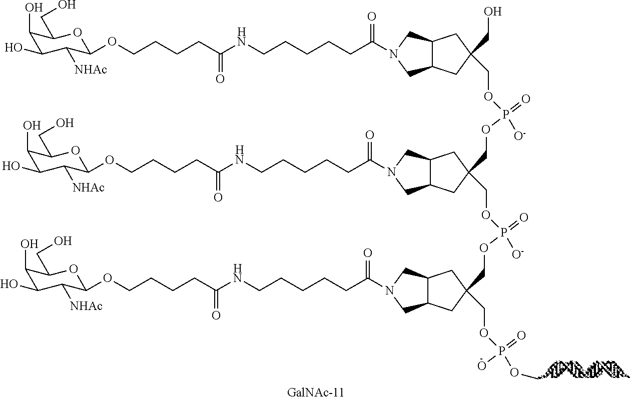 [SMILES: COP(=O)(O)OC[C@]1(COP(=O)([O-])OC[C@]2(COP(=O)([O-])OC[C@]3(CO)C[C@H]4CN(C(=O)CCCCCNC(=O)CCCCOC5OC(CO)C(O)C(O)C5NC(C)=O)C[C@H]4C3)C[C@H]3CN(C(=O)CCCCCNC(=O)CCCCOC4OC(CO)C(O)C(O)C4NC(C)=O)C[C@H]3C2)C[C@H]2CN(C(=O)CCCCCNC(=O)CCCCOC3OC(CO)C(O)C(O)C3NC(C)=O)C[C@H]2C1]

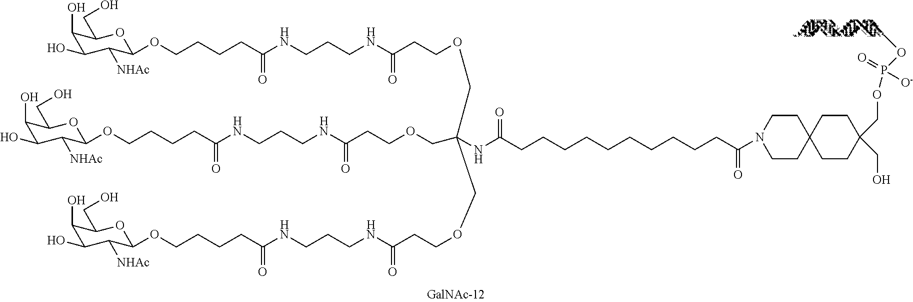 [SMILES: COP(=O)([O-])OCC1(CO)CCC2(CCN(C(=O)CCCCCCCCCCC(=O)NC(COCCC(=O)NCCCNC(=O)CCCCOC3OC(CO)C(O)C(O)C3NC(C)=O)(COCCC(=O)NCCCNC(=O)CCCCOC3OC(CO)C(O)C(O)C3NC(C)=O)COCCC(=O)NCCCNC(=O)CCCCOC3OC(CO)C(O)C(O)C3NC(C)=O)CC2)CC1]

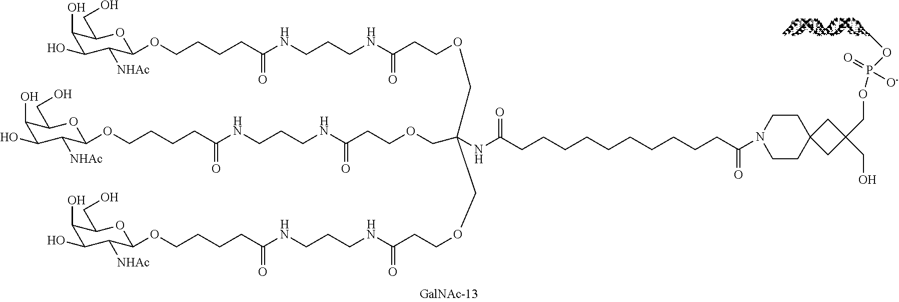 [SMILES: COP(=O)([O-])OCC1(CO)CC2(CCN(C(=O)CCCCCCCCCCC(=O)NC(COCCC(=O)NCCCNC(=O)CCCCOC3OC(CO)C(O)C(O)C3NC(C)=O)(COCCC(=O)NCCCNC(=O)CCCCOC3OC(CO)C(O)C(O)C3NC(C)=O)COCCC(=O)NCCCNC(=O)CCCCOC3OC(CO)C(O)C(O)C3NC(C)=O)CC2)C1]

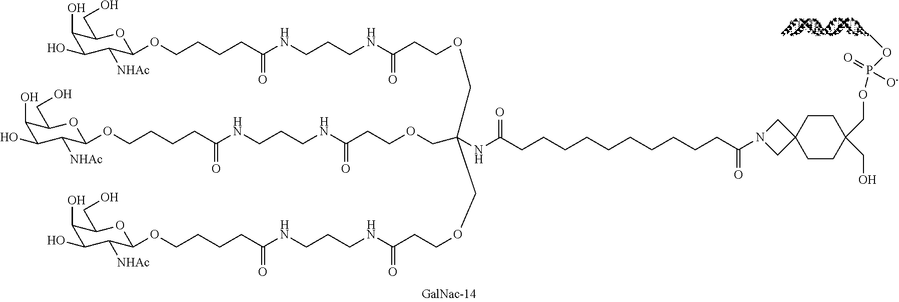 [SMILES: COP(=O)([O-])OCC1(CO)CCC2(CC1)CN(C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O)C2]

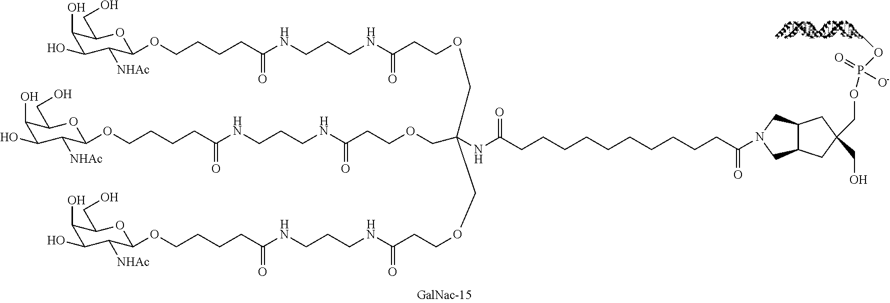 [SMILES: COP(=O)([O-])OC[C@]1(CO)C[C@H]2CN(C(=O)CCCCCCCCCCC(=O)NC(COCCC(=O)NCCCNC(=O)CCCCOC3OC(CO)C(O)C(O)C3NC(C)=O)(COCCC(=O)NCCCNC(=O)CCCCOC3OC(CO)C(O)C(O)C3NC(C)=O)COCCC(=O)NCCCNC(=O)CCCCOC3OC(CO)C(O)C(O)C3NC(C)=O)C[C@H]2C1]

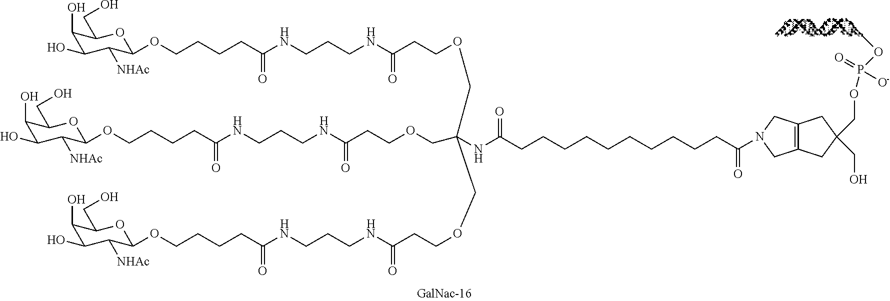 [SMILES: COP(=O)([O-])OCC1(CO)CC2=C(CN(C(=O)CCCCCCCCCCC(=O)NC(COCCC(=O)NCCCNC(=O)CCCCOC3OC(CO)C(O)C(O)C3NC(C)=O)(COCCC(=O)NCCCNC(=O)CCCCOC3OC(CO)C(O)C(O)C3NC(C)=O)COCCC(=O)NCCCNC(=O)CCCCOC3OC(CO)C(O)C(O)C3NC(C)=O)C2)C1]

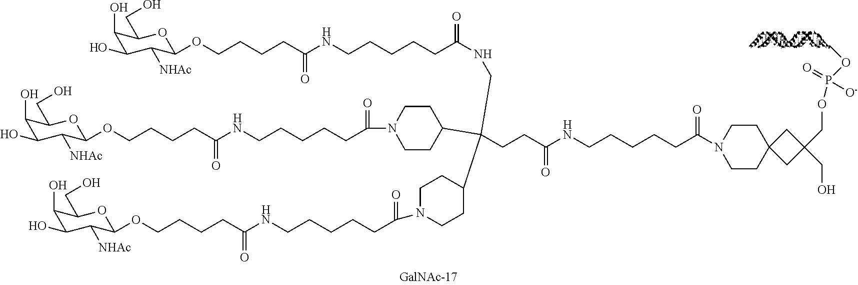 [SMILES: COP(=O)([O-])OCC1(CO)CC2(CCN(C(=O)CCCCCNC(=O)CCC(CNC(=O)CCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)CC2)C1]

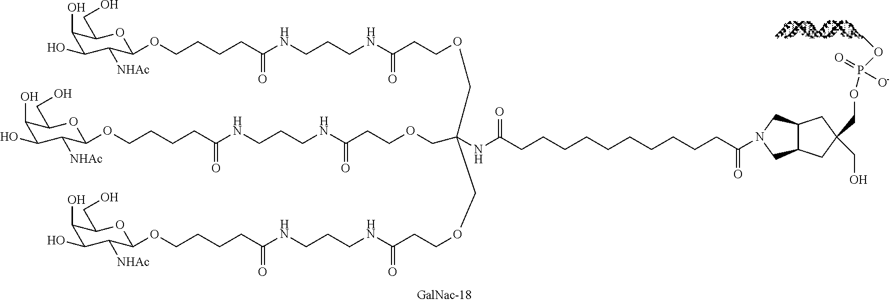 [SMILES: COP(=O)([O-])OC[C@@]1(CO)C[C@H]2CN(C(=O)CCCCCCCCCCC(=O)NC(COCCC(=O)NCCCNC(=O)CCCCOC3OC(CO)C(O)C(O)C3NC(C)=O)(COCCC(=O)NCCCNC(=O)CCCCOC3OC(CO)C(O)C(O)C3NC(C)=O)COCCC(=O)NCCCNC(=O)CCCCOC3OC(CO)C(O)C(O)C3NC(C)=O)C[C@H]2C1]

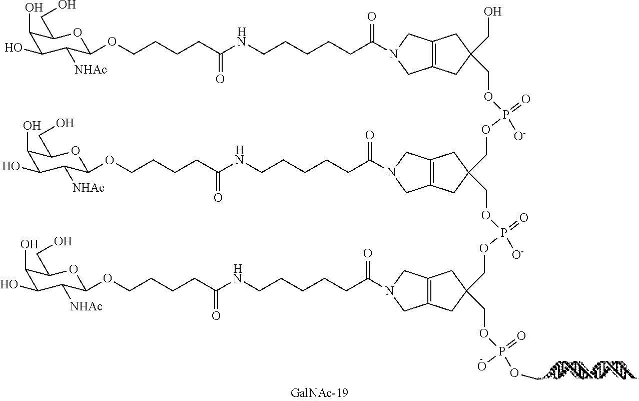 [SMILES: COP(=O)(O)OCC1(COP(=O)([O-])OCC2(COP(=O)([O-])OCC3(CO)CC4=C(CN(C(=O)CCCCCNC(=O)CCCCOC5OC(CO)C(O)C(O)C5NC(C)=O)C4)C3)CC3=C(CN(C(=O)CCCCCNC(=O)CCCCOC4OC(CO)C(O)C(O)C4NC(C)=O)C3)C2)CC2=C(CN(C(=O)CCCCCNC(=O)CCCCOC3OC(CO)C(O)C(O)C3NC(C)=O)C2)C1]

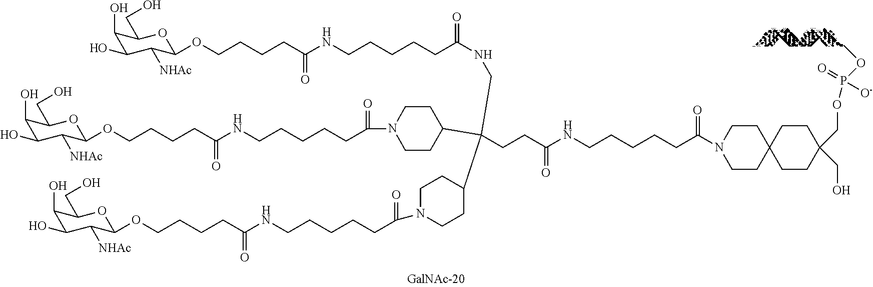 [SMILES: COP(=O)([O-])OCC1(CO)CCC2(CCN(C(=O)CCCCCNC(=O)CCC(CNC(=O)CCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)CC2)CC1]

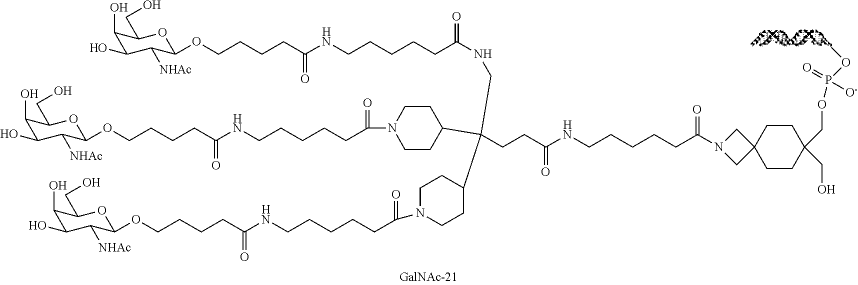 [SMILES: COP(=O)([O-])OCC1(CO)CCC2(CC1)CN(C(=O)CCCCCNC(=O)CCC(CNC(=O)CCCCCNC(=O)CCCCOC1OC(CO)C(O)C(O)C1NC(C)=O)(C1CCN(C(=O)CCCCCNC(=O)CCCCOC3OC(CO)C(O)C(O)C3NC(C)=O)CC1)C1CCN(C(=O)CCCCCNC(=O)CCCCOC3OC(CO)C(O)C(O)C3NC(C)=O)CC1)C2]

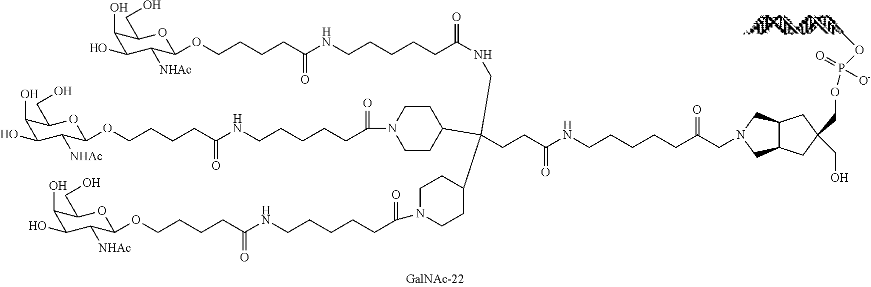 [SMILES: COP(=O)([O-])OC[C@@]1(CO)C[C@H]2CN(CC(=O)CCCCCNC(=O)CCC(CNC(=O)CCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)C[C@H]2C1]

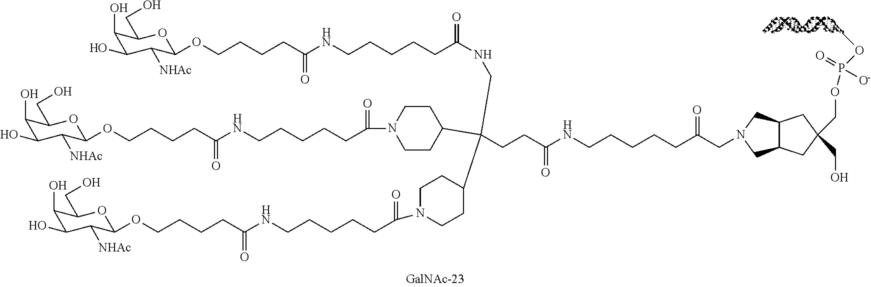 [SMILES: COP(=O)([O-])OC[C@]1(CO)C[C@H]2CN(CC(=O)CCCCCNC(=O)CCC(CNC(=O)CCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)C[C@H]2C1]

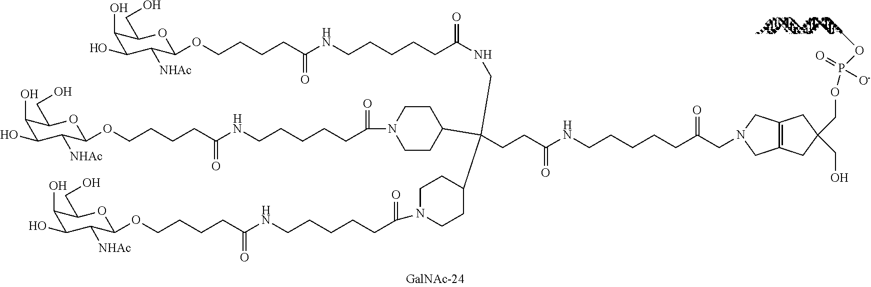 [SMILES: COP(=O)([O-])OCC1(CO)CC2=C(CN(CC(=O)CCCCCNC(=O)CCC(CNC(=O)CCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)C2)C1]

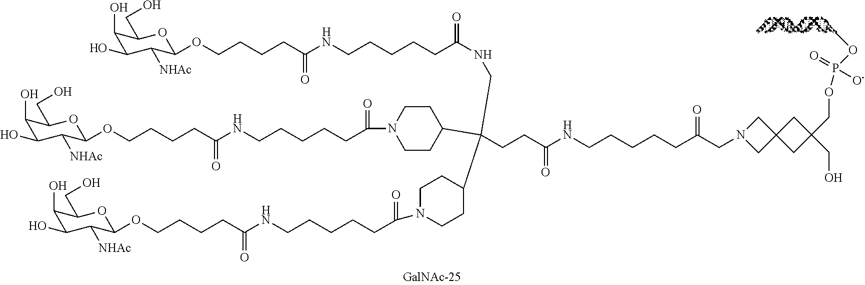 [SMILES: COP(=O)([O-])OCC1(CO)CC2(CN(CC(=O)CCCCCNC(=O)CCC(CNC(=O)CCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)C2)C1]

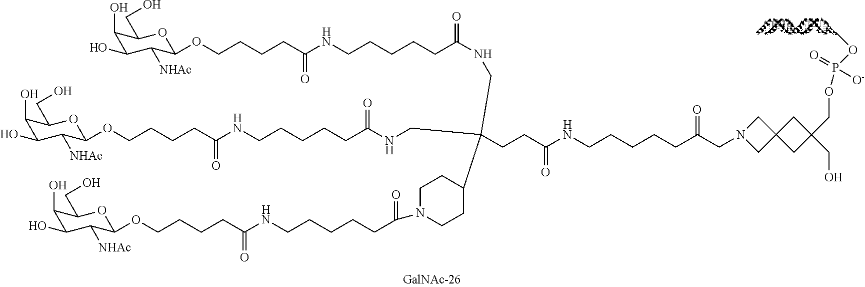 [SMILES: COP(=O)([O-])OCC1(CO)CC2(CN(CC(=O)CCCCCNC(=O)CCC(CNC(=O)CCCCCNC(=O)CCCCOC3OC(CO)C(O)C(O)C3NC(C)=O)(CNC(=O)CCCCCNC(=O)CCCCOC3OC(CO)C(O)C(O)C3NC(C)=O)C3CCN(C(=O)CCCCCNC(=O)CCCCOC4OC(CO)C(O)C(O)C4NC(C)=O)CC3)C2)C1]

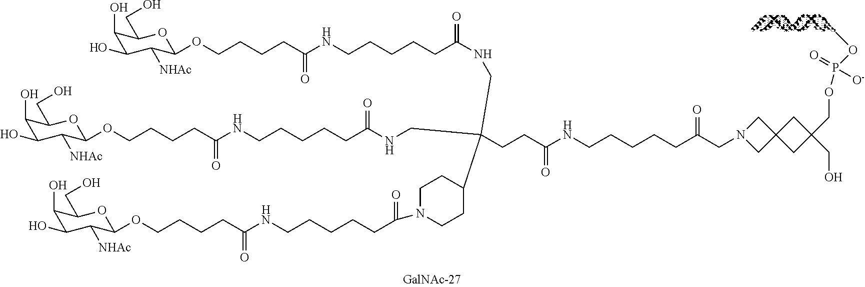 [SMILES: COP(=O)([O-])OCC1(CO)CC2(CN(CC(=O)CCCCCNC(=O)CCC(CNC(=O)CCCCCNC(=O)CCCCOC3OC(CO)C(O)C(O)C3NC(C)=O)(CNC(=O)CCCCCNC(=O)CCCCOC3OC(CO)C(O)C(O)C3NC(C)=O)C3CCN(C(=O)CCCCCNC(=O)CCCCOC4OC(CO)C(O)C(O)C4NC(C)=O)CC3)C2)C1]

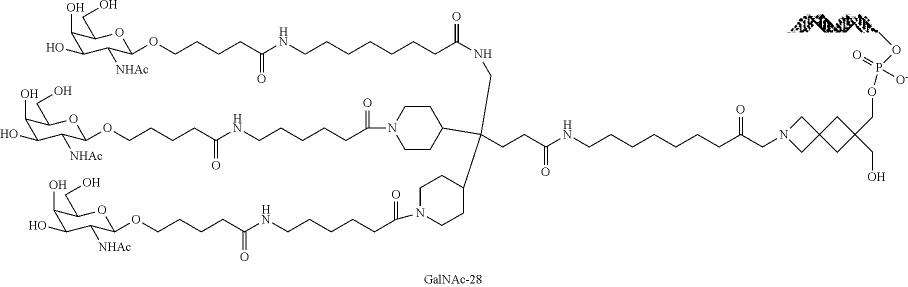 [SMILES: COP(=O)([O-])OCC1(CO)CC2(CN(CC(=O)CCCCCCCNC(=O)CCC(CNC(=O)CCCCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)C2)C1]

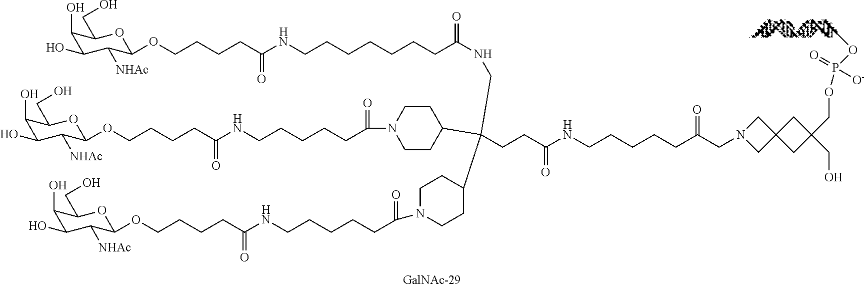 [SMILES: COP(=O)([O-])OCC1(CO)CC2(CN(CC(=O)CCCCCNC(=O)CCC(CNC(=O)CCCCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)C2)C1]

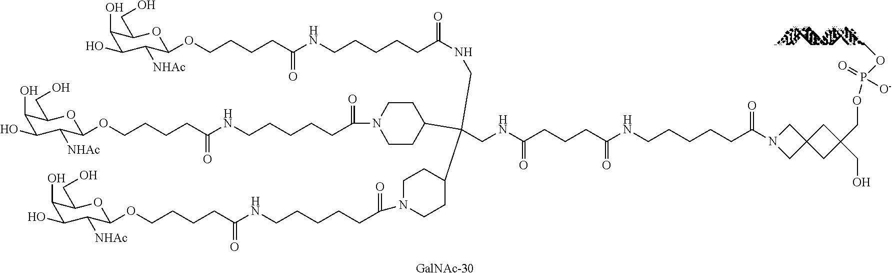 [SMILES: COP(=O)([O-])OCC1(CO)CC2(CN(C(=O)CCCCCNC(=O)CCCC(=O)NCC(CNC(=O)CCCCCNC(=O)CCCCOC3OC(CO)C(O)C(O)C3NC(C)=O)(C3CCN(C(=O)CCCCCNC(=O)CCCCOC4OC(CO)C(O)C(O)C4NC(C)=O)CC3)C3CCN(C(=O)CCCCCNC(=O)CCCCOC4OC(CO)C(O)C(O)C4NC(C)=O)CC3)C2)C1]

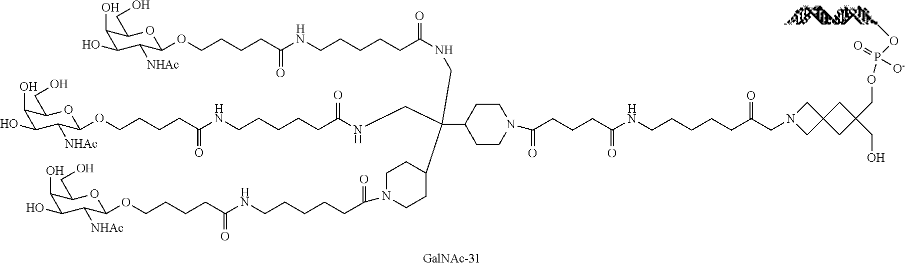 [SMILES: COP(=O)([O-])OCC1(CO)CC2(CN(CC(=O)CCCCCNC(=O)CCCC(=O)N3CCC(C(CNC(=O)CCCCCNC(=O)CCCCOC4OC(CO)C(O)C(O)C4NC(C)=O)(CNC(=O)CCCCCNC(=O)CCCCOC4OC(CO)C(O)C(O)C4NC(C)=O)C4CCN(C(=O)CCCCCNC(=O)CCCCOC5OC(CO)C(O)C(O)C5NC(C)=O)CC4)CC3)C2)C1]

### Biological Assays

GalNAc conjugated siRNAs against human Proprotein Convertase Subtilisin/Kexin Type 9 (hPCSK9), mouse complement component 3 (mC3), or mouse Transthyretin (mTTR) genes in this invention were tested for free uptake in the primary human hepatocytes (PHH), in the primary mouse hepatocytes (PMH), and in wild type mice. The efficacy was determined by the reduction of hPCSK9, mC3, or mTTR mRNA level using the real time quantitative RT-PCR assay.

GalNAc conjugated siRNAs against hPCSK9 gene in this invention were also examined for competition activity with a reporter siRNA-8 (GalNAc-1 conjugated siRNA labeled with Cy3 dye) to bind with asialoglycoprotein receptors (ASGPR) in human liver cancer cell line, HepG2. The competition activity was determined by the reduction of mean fluorescence intensity in the cells, measured by the flow cytometry.

### 1. Cell Culture

Human HepG2 cell was purchased from ATCC and was passaged at 70% confluency in the culture media, DMEM (Invitrogen) supplemented with 10% Fetal Bovine Serum (FBS, Invitrogen).

Primary human hepatocytes (PHH) purchased as frozen vials from Invitrogen were thawed in Cryopreserved Hepatocyte Recovery Medium (CHRM, Gibco) and plated with plating medium (William Medium E (Gibco, A1217601) supplemented with Hepatocyte Plating Supplement Pack (Gibco, CM3000)) for 4-6 hrs in collagen I coated 96-well plates (Gibco, A1142803) at the density of about 1.0×10⁵ cells/100 ul medium/well. Then, the plating medium was changed to incubation medium (Williams Medium E (Gibco, A1217601) supplemented with Hepatocyte Maintenance Supplement Pack (Gibco, CM4000)), with the treatment of GalNAc conjugated siRNA for free uptake.

PMH purchased as frozen vials from Xenotech were thawed in OptiThaw hepatocyte media and plated with plating medium (OptiPlate hepatocyte media) for 4-6 hrs in collagen I coated 96-well plates (Gibco, A1142803) at the density of about 1.0×10⁵ cells/100 ul medium/well. Then, the plating medium was changed to incubation medium (OptiCulture media), with the treatment of GalNAc conjugated siRNA for free uptake.

2. siRNAs for hPCSK9, mC3, and mTTR (Example 32)

siRNA for hPCSK9 was synthesized by solid phase oligonucleotide synthesis according to the following published sequences:

Sense strand 5′-3′:
CUAGACCUGUTUUGCUUUUGU
antisense strand 3′-5′:
AAGAUCUGGACAAAACGAAAACA

siRNA for mC3 was synthesized by solid phase oligonucleotide synthesis according to the following published sequences:

Sense strand 5′-3′:
UGGUCAAGGUCUUCUCUCU
antisense strand 3′-5′:
ACCAGUUCCAGAAGAGAGU

siRNA for mTTR was synthesized by solid phase oligonucleotide synthesis according to the following published sequences:

Sense strand 5′-3′:
AACAGUGUUCUUGCUCUAUAA
antisense strand 3′-5′:
UUUUGUCACAAGAACGAGAUAUU

### 3. Free Uptake

Plating medium of PHH or PMH cells as indicated was changed to pre-warmed incubation medium with siRNAs at multiple final concentrations of 100, 20, 4, 0.8, 0.16, 0.032, 0.0064, 0.00128, 0.000256 nM. After incubation for 48 hours, mRNA was extracted for RT-qPCR.

4. mRNA Isolation

mRNA of PHH or PMH was isolated by using Dynabeads™ mRNA DIRECT™ Purification Kit (Invitrogen, Cat #61012). Incubation medium was discarded and 100 ul of lysis/binding buffer provided in the kit was added to the cells. The lysate of cells was collected after 15-30 mins incubation at room temperature followed by mixing with the magnetic Dynabeads. After 5 mins incubation, Dynabeads were washed with buffers provided in the kit, air dried and then processed with cDNA synthesis.

5. cDNA Synthesis

Following mRNA isolation, reverse transcription was done immediately by SuperScript VILO cDNA Synthesis Kit (Invitrogen life technology).

### 6. Gene Expression Analysis (RT-qPCR).

All probes for RNA quantification were acquired from Life Technologies utilizing their TaqMan gene expression system with dual-labeled probes. Target gene expression was normalized to TATA-box binding protein (TBP) as a control in each well. Ct values were measured using Quantstudio 5 real time PCR system (Invitrogen life technology). To calculate relative fold change, real time data was analyzed using the comparative CT method and normalized to relative target mRNA levels in untreated cells. TaqMan probe catalogue numbers are shown as following

Target genes:
Hunan PCSK9-FAM (Assay ID: Hs00545399_m1)
Mouse C3-FAM (Assay ID: Mm01232779_m1)
Mouse TTR-FAM (Assay ID: Mm00443267_m1)
Housekeeping genes:
Human TBP-VIC (Assay ID: Hs00427620_m1)
Mouse TBP-VIC (Assay ID: Mm01277042_m1)

### Example 1. Triantennary GalNAc-siRNAs Free Uptake Assay in PHH

The triantennary GalNAc conjugated siRNAs targeting hPCSK9 mRNA were assayed for target mRNA knockdown activity in PHH. Plateable human cryopreserved primary hepatocytes were purchased from Gibco (Cat #: HMCPUS, Lot #: Hu8115). Cells were thawed immediately in 37° C., after taking from liquid nitrogen, then, transferred to pre-warmed thawing medium (Gibco, CM7000) before complete melting. Cells were centrifuged at 100 g for 10 mins at room temperature, removed the thawing medium, and added plating medium, which is William Medium E (Gibco, A1217601) supplemented with Hepatocyte Plating Supplement Pack (Gibco, CM3000) to plate cells in collagen I coated 96-well plates (Gibco, A1142803) at the density of about 1.0×10⁵ cells/100 ul medium/well. After incubation in 37° C. for 4 hours, plating medium was changed to incubation medium (Williams Medium E (Gibco, A1217601) supplemented with Hepatocyte Maintenance Supplement Pack (Gibco, CM4000)), containing novel GalNAc conjugated siRNAs with 5-fold dilution from 100 nM stepped down to 0.000256 nM, except siRNA-14, -16 with 3-fold dilution from 100 nM stepped down to 0.01 nM. Two biological replicates were done for the data. Cells were harvested 48 hours post siRNA addition, followed by mRNA RNA isolation by Dynabeads™ mRNA DIRECT™ Purification Kit (Invitrogen, Cat #61012) and cDNA synthesis by SuperScript VILO cDNA Synthesis Kit (Invitrogen life technology) according to the manufacturer's instructions. The level of hPCSK9 mRNA in each well was measured using TaqMan Gene Expression Assay (ThermoFisher), normalized to hTBP mRNA, and in duplicate by real-time qPCR on a Quantstudio 5 real time PCR system (Invitrogen life technology). Data was calculated by using the delta-delta Ct method and was normalized to relative target mRNA levels in untreated cells. Dose-response curves were made with GraphPad Prism version 9.3.1 using a four-parameter logistic model.

The data show the maximum effect of novel GalNAc conjugates on hPCSK9 mRNA level. The GalNAc moiety L96 (siRNA-1) was used for a positive control in the assay. If the remained hPCSK9 mRNA levels in the GalNAc conjugate treatments were lower than L96, we think the novel GacNAcs are better than siRNA-1. Otherwise, they are worse than siRNA-1. The rank for all triantennary GalNAcs shown in Table 2 was determined by the average maximum effect at 100 nM in the two biological replicates. The fold of maximum effect was calculated by ratio from the percentage of remained mRNA from the cells treated with siRNA-1 divided by other GalNAc-siRNA treatments. The results of free uptake assay in PHH show that designed triantennary GalNAc with the maximum effect around 35% of remained target mRNA are comparable to siRNA-1.

TABLE 2
Rank of triantennary GalNAcs based on maximum
effect in PHH free uptake assay.

| | GalNAc type | | 100 nM |
|---|---|---|---|
| | and position | 100 nM Average | (Average fold, |
| siRNA ID | at sense strand | percentage | siRNA-1/X) |
| siRNA-14 | GalNAc-12 at 3′ end | 21.61 | 1.60 |
| siRNA-16 | GalNAc-14 at 3′ end | 26.21 | 1.32 |
| siRNA-15 | GalNAc-13 at 3′ end | 28.17 | 1.23 |
| siRNA-17 | GalNAc-15 at 3′ end | 28.51 | 1.22 |
| siRNA-19 | GalNAc-17 at 3′ end | 32.17 | 1.08 |
| siRNA-18 | GalNAc-16 at 3′ end | 32.99 | 1.05 |
| siRNA-20 | GalNAc-18 at 3′ end | 34.34 | 1.01 |
| siRNA-21 | GalNAc-7 at 5′ end | 34.35 | 1.01 |
| siRNA-1 | GalNAc-1 at 3′ end | 34.66 | 1.00 |
| siRNA-7 | GalNAc-7 at 3′ end | 34.88 | 0.99 |
| siRNA-6 | GalNAc-6 at 3′ end | 35.98 | 0.96 |
| siRNA-30 | GalNAc-20 at 3′ end | 48.12 | 0.72 |
| siRNA-31 | GalNAc-21 at 3′ end | 55.90 | 0.62 |
| siRNA-33 | GalNAc-23 at 3′ end | 59.50 | 0.58 |
| siRNA-32 | GalNAc-22 at 3′ end | 60.41 | 0.57 |
| siRNA-34 | GalNAc-24 at 3′ end | 66.23 | 0.52 |
| siRNA-41 | GalNAc-24 at 3′ end | 67.81 | 0.47 |

### Example 2. (1+1+1) Trivalent GalNAc-siRNAs Free Uptake Assay in PHH

The (1+1+1) trivalent GalNAc conjugated siRNAs targeting hPCSK9 mRNA were assayed for target mRNA knockdown activity in PHH. The methods and procedures for PHH free uptake, mRNA isolation, and RT-qPCR were completely same as triantennary GalNAc conjugated siRNAs as described above. The rank of (1+1+1) trivalent GalNAc conjugates is shown in Table 3. The ranking is based on the maximum effect on remained target mRNA percentage. The data of siRNA-treated cells at 100 nM show the maximum effect of novel GalNAc conjugates on hPCSK9 mRNA level. The positive controls are siRNA-2 and siRNA-29.

TABLE 3
Rank of (1 + 1 + 1) trivalent GalNAcs based
on maximum effect in PHH free uptake assay.

| | GalNAc type | | 100 nM | 100 nM |
|---|---|---|---|---|
| | and position | 100 nM Average | (Average fold, | (Average fold, |
| siRNA ID | at sense strand | percentage | siRNA-2/X) | siRNA-29/X) |
| siRNA-5 | GalNAc-5 at 5′ end | 22.00 | 3.01 | 3.05 |
| siRNA-3 | GalNAc-3 at 3′ end | 28.22 | 2.16 | 2.38 |
| siRNA-13 | GalNAc-3 at 5′ end | 31.03 | 1.71 | 2.16 |
| siRNA-12 | GalNAc-11 at 5′ end | 34.29 | 1.59 | 1.96 |
| siRNA-11 | GalNAc-10 at 5′ end | 37.04 | 1.91 | 1.81 |
| siRNA-22 | GalNAc-19 at 5′ end | 47.27 | 1.12 | 1.42 |
| siRNA-2 | GalNAc-2 at 3′ end | 52.85 | 1.00 | 1.27 |
| siRNA-9 | GalNAc-8 at 5′ end | 55.99 | 0.98 | 1.20 |
| siRNA-10 | GalNAc-9 at 5′ end | 65.09 | 0.82 | 1.03 |
| siRNA-29 | GalNAc-2 at 5′ end | 67.15 | 0.80 | 1.00 |

The result of free uptake assay in PHH shows that designed (1+1+1) trivalent GalNAc conjugates with the maximum effect on remaining target mRNA after knocking down by less than 50% are more potent to siRNA-2 and siRNA-29 (controls). siRNA-5 and siRNA-3 inhibited target mRNA as low as less than 30%, although with the same siRNA sequences and chemical modifications as others.

### Example 3. Competition Assay to Detect the Uptake Activity of GalNAcs in HepG2 Cells

GalNAc is a high affinity ligand for ASGPR. To further compare ASGPR mediated uptake and potency of different GalNAc conjugated siRNAs, we used Cy3-labeled GalNAc-siRNA (siRNA-8) as a reporter and mixed with different concentration of GalNAc-siRNAs by measurement of siRNA-8 internalization in liver cells by flow cytometry. As the amount of GalNAc-siRNA increased, the internalization of the reporter decreased. We chose siRNAs with more or less efficient GalNAc compared to positive controls in PHH free uptake assays for the competition assay. HepG2 cells were seeded in 384-well plates (1×10⁴ cells/well) and allowed to attach and equilibrate for at least 16 h at 37° C. The Cy3-labeled GalNAc-siRNA (siRNA-8) was mixed with 8-point 3-fold diluted designed GalNAc-siRNAs in the culture media. The HepG2 cells were placed on ice for siRNA treatment. The old media of HepG2 cells was changed with the media containing siRNAs, and cells were continuously cultured at 37° C. for 2 h. Following the incubation, period cells were placed on ice and washed three times with ice-cold PBS (pH=7.4, ThermoFisher), and lifted with trypsin at 37° C. for 10 min followed by ice-cold media to stop trypsin treatment. Cells were then transferred to new 96-well plates and the media were changed to EasySep™ Buffer (Stemcell Technologies) on ice. Cells then were analyzed by flow cytometry for mean fluorescence intensity using a BD LSRFortessa™ X-20 (BD Biosciences). Internalized siRNA was expressed as mean fluorescence intensity (MFI). Dose-response curves were made with GraphPad Prism version 9.3.1 using a four-parameter logistic model.

TABLE 4
Rank of triantennary GalNAcs based on
maximum effect in competition assay.

| | GalNAc type | Maximum | Maximum | SPR |
|---|---|---|---|---|
| | and position | effect | effect (fold, | K_{D} |
| siRNA ID | at sense strand | (%) | siRNA-1/X) | (nM) |
| siRNA-32 | GalNAc-22 at 3′ end | 36.50 | 1.24 | 2.48 |
| siRNA-6 | GalNAc-6 at 3′ end | 39.36 | 1.15 | 5.85 |
| siRNA-14 | GalNAc-12 at 3′ end | 40.24 | 1.13 | 3.95 |
| siRNA-7 | GalNAc-7 at 3′ end | 41.32 | 1.10 | 3.77 |
| siRNA-17 | GalNAc-15 at 3′ end | 42.07 | 1.08 | 4.66 |
| siRNA-1 | GalNAc-1 at 3′ end | 45.40 | 1.00 | 5.91 |
| siRNA-15 | GalNAc-13 at 3′ end | 47.64 | 0.95 | 4.42 |

TABLE 5
Rank of (1 + 1 + 1) trivalent GalNAcs based
on maximum effect in competition assay.

| | GalNAc type | Maximum | Maximum | SPR |
|---|---|---|---|---|
| | and position | effect | effect (fold, | K_{D} |
| siRNA ID | at sense strand | (%) | siRNA-2/X) | (nM) |
| siRNA-5 | GalNAc-5 at 5′ end | 66.21 | 1.51 | 4.13 |
| siRNA-11 | GalNAc-10 at 5′ end | 70.59 | 1.42 | 3.96 |
| siRNA-3 | GalNAc-3 at 3′ end | 89.26 | 1.12 | 4.85 |
| siRNA-2 | GalNAc-2 at 3′ end | 100.14 | 1.00 | 6.76 |

In parallel, binding affinity of GalNAc-siRNA to ASGPR was tested by Surface Plasmon Resonance (SPR) assay on Biacore 8K machine (Biacore AB), equipped by NiHC200M Chip). Here, in-house made his-tagged human ASPGR1 CRD domain of H1 subunit (aa 154-281) was immobilized to the chip. The results of binding affinity were also shown in Tables 4 and 5.

The result of competition assay shows that all of triantennary GalNAc conjugates could inhibit siRNA-8 internalization at the 6 uM from 36% to 47% and had similar binding affinity to ASGPR tested by SPR.

The result of competition assay shows that siRNA-2 could not inhibit Cy3-labeled GalNAc-siRNA (siRNA-8) internalization at the 6 uM, while other siRNAs with different (1+1+1) trivalent GalNAc could inhibit and had similar binding affinity to ASGPR tested by SPR.

### Example 4. Triantennary and (1+1+1) Trivalent GalNAc-siRNAs in Free Uptake Assays in PMH

We chose two triantennary GalNAcs (GalNAc-6 at 3′ end of sense strand and GalNac-7 at 3′ sense strand) for further analysis in PMH free uptake assays. siRNA-24 and siRNA-27 (conjugated with GalNAc-6 at 3′ end of sense strand) are targeting mC3 and mTTR respectively. siRNA-25 and siRNA-28 (conjugated with GalNAc-7 at 3′ end of sense strand) are targeting mC3 and mTTR mRNAs, respectively.

We also chose three (1+1+1) trivalent GalNAcs (GalNAc-3 at 5′ end of sense strand, and GalNAc-5 at 5′ end of sense strand) for further analysis in PMH free uptake assays. The three (1+1+1) trivalent GalNAcs are conjugated to siRNA-37, and siRNA-38, respectively, which are targeting mTTR mRNA.

The triantennary or (1+1+1) trivalent GalNAc conjugated siRNAs targeting mC3 mRNA or mTTR were assayed for target mRNA knockdown activity in PMH. Cells were thawed immediately in 37° C., after taking from liquid nitrogen, then, transferred to pre-warmed OptiThaw hepatocyte media before complete melting. Cells were centrifuged at 100 g for 10 mins at room temperature, removed the thawing medium, and added OptiPlate hepatocyte media to plate cells in collagen I coated 96-well plates (Gibco, A1142803) at the density of about 1.0×10⁵ cells/100 ul medium/well. After incubation in 37° C. for 4 hours, plating medium was changed to OptiCulture media, containing novel GalNAc conjugated siRNAs with 5-fold dilution from 100 nM stepped down to 0.000256 nM. Cells were harvested 48 hours post siRNA addition, followed by mRNA RNA isolation by Dynabeads™ mRNA DIRECT™ Purification Kit (Invitrogen, Cat #61012) and cDNA synthesis by SuperScript VILO CDNA Synthesis Kit (Invitrogen life technology) according to the manufacturer's instructions. The level of mC3 mRNA in each well was measured using TaqMan Gene Expression Assay (ThermoFisher), normalized to mTBP mRNA, and in duplicate by real-time qPCR on a Quantstudio 5 real time PCR system (Invitrogen life technology). Date was calculated by using the delta-delta Ct method and was normalized to relative target mRNA levels in untreated cells. Dose-response curves were made with GraphPad Prism version 9.3.1 using a four-parameter logistic model.

TABLE 6
Rank of triantennary GalNAcs based on maximum effect
in PMH free uptake assay (targeting mC3 mRNA).

| | GalNAc type | Maximum | |
|---|---|---|---|
| | and position | effect | Maximum effect |
| siRNA ID | at sense strand | (%) | (fold, siRNA-23/X) |
| siRNA-24 | GalNAc-6 at 3′ end | 4.72 | 2.04 |
| siRNA-25 | GalNAc-7 at 3′ end | 8.49 | 1.13 |
| siRNA-23 | GalNAc-1 at 3′ end | 9.63 | 1.00 |

TABLE 7
Rank of triantennary GalNAcs based on maximum effect
in PMH free uptake assay (targeting mTTR mRNA).

| | GalNAc type | Maximum | |
|---|---|---|---|
| | and position | effect | Maximum effect |
| siRNA ID | at sense strand | (%) | (fold, siRNA-26/X) |
| siRNA-27 | GalNAc-6 at 3′ end | 0.30 | 1.62 |
| siRNA-28 | GalNAc-7 at 3′ end | 0.46 | 1.07 |
| siRNA-26 | GalNAc-1 at 3′ end | 0.49 | 1.00 |

TABLE 8
Rank of 1 + 1 + 1 GalNAcs based on maximum effect
in PMH free uptake assay (targeting mTTR mRNA).

| | GalNAc type | Maximum | Maximum |
|---|---|---|---|
| | and position | effect | effect (fold, |
| siRNA ID | at sense strand | (%) | siRNA-40/X) |
| siRNA-37 | GalNAc-3 at 5′ end | 0.20 | 1.61 |
| siRNA-40 | GalNAc-2 at 5′ end | 0.32 | 1.00 |
| siRNA-38 | GalNAc-5 at 5′ end | 0.37 | 0.87 |

The result in Tables 6 and 7 shows the designed triantenanry GalNAc are more potent than the controls (siRNA-23 and siRNA-26) and the result in Table 8 shows the designed (1+1+1) trivalent GalNAc are more potent than the control (siRNA-40).

### Example 5. Triantennary and (1+1+1) Trivalent GalNAc-siRNAs In Vivo Studies

### In Vivo Study Procedure

Male C57BL/6 mice were obtained from Jackson Laboratories at around 6-8 weeks of age. Animals were weighed and then assigned to each group to ensure the average and variation of body weights in each group were similar. Testing articles (GalNAc-conjugated siRNA) were prepared in sterile phosphate-buffered saline (PBS) at 1 mg/mL. siRNA concentration and quality were measured using QIAxpert (Qiagen). Mice were dosed subcutaneously at 5 mL/kg with siRNAs or PBS control (vehicle). Blood was collected using the retro-orbital eye bleed procedure and then subjected to serum preparation. After obtaining the serum, it was transferred to a 1.5 mL tube for storage at −80° C. Baseline blood samples were collected 2 days before the injection. Blood was then collected again on Day 7 post-administration. Animals were sacrificed on day 14, followed by blood collection and liver harvest. One lobe of the liver was stored in 2.5 mL of RNAlater (Thermo Fisher Scientific) and kept at 4° C. for RNA extraction later. Another lobe of the liver was snap-frozen and kept at −80° C. For serum proteins, the mTTR level was measured by the ELISA kit from LS Bio (Cat. No. LS-F7530), and the mC3 level was measured by the ELISA kit from Abcam (Cat. No. ab263884).

For liver gene expression, liver tissues in RNAlater were homogenized, and then mRNAs were isolated with Dynabeads® mRNA DIRECT™ Purification Kit (Thermo Fisher Scientific). cDNA productions were performed with SuperScript™ IV VILO™ Master Mix (Thermo Fisher Scientific). Real-Time PCR reactions were done using gene-specific TaqMan assays (Thermo Fisher Scientific).

### In Vivo Results Summary

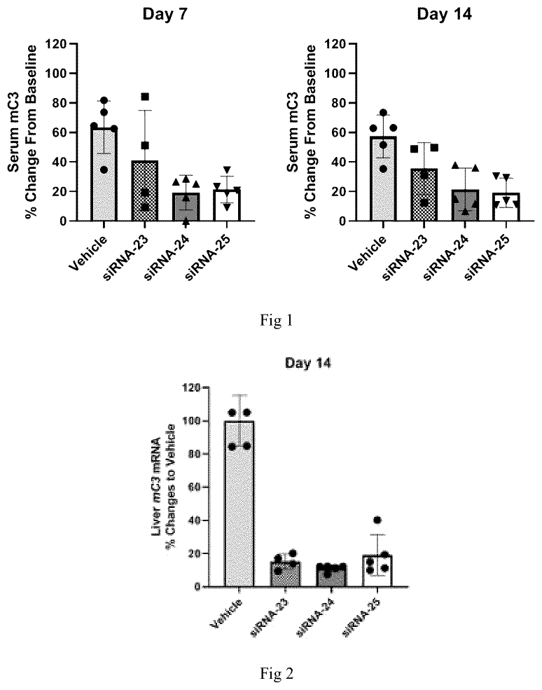

After administration, siRNA-23, siRNA-24, and siRNA-25 showed sustainable serum mC3 protein reduction up to the last time point examined (day 14). In siRNA-24 or siRNA-25 treated groups, mC3 protein levels are statistically significantly lower than the vehicle-treated group on both day 7 and day 14. siRNA-24 and siRNA-25 showed a trend of better mC3 knock-down when compared with siRNA-23 (FIG. 1). In addition, siRNA-24, and siRNA-25 significantly decreased the mC3 mRNA levels in the liver on Day 14 when compared to the vehicle group (FIG. 2).

| | | Vehicle | siRNA-23 | siRNA-24 | siRNA-25 |
|---|---|---|---|---|---|
| Liver mC3 | Day 14 | 100.00 | 15.23 | 11.07 | 19.20 |
| mRNA % | | | | | |
| Serum mC3 | Day 7 | 63.41 | 40.94 | 19.25 | 21.27 |
| Protein % | Day 14 | 57.22 | 35.43 | 21.42 | 19.05 |

Similarly, continuous knock-down of mTTR protein by siRNA-37, -38, -39, and -40 are observed up to day 14 post-dose. All siRNAs significantly decreased mTTR protein levels on days 7 and 14 and mTTR mRNA levels on days 14 compared with the vehicle-treated group.

| | | | siRNA- | siRNA- | siRNA- | siRNA- |
|---|---|---|---|---|---|---|
| | | Vehicle | 37 | 38 | 39 | 40 |
| Liver | Day 14 | 100.00 | 2.99 | 5.19 | 8.68 | 6.73 |
| mTTR | | | | | | |
| mRNA % | | | | | | |
| Serum | Day 7 | 125.43 | 0.37 | 0.38 | 1.28 | 0.33 |
| mTTR | Day 14 | 106.87 | 1.42 | 0.75 | 2.85 | 1.57 |
| Protein % | | | | | | |

Our data indicated that the designed GalNAc conjugates have good expression knock-down.

1. A targeting ligand having the structure shown in formula (III-b), (III-c) or (III-d) or (III) or (III-a): 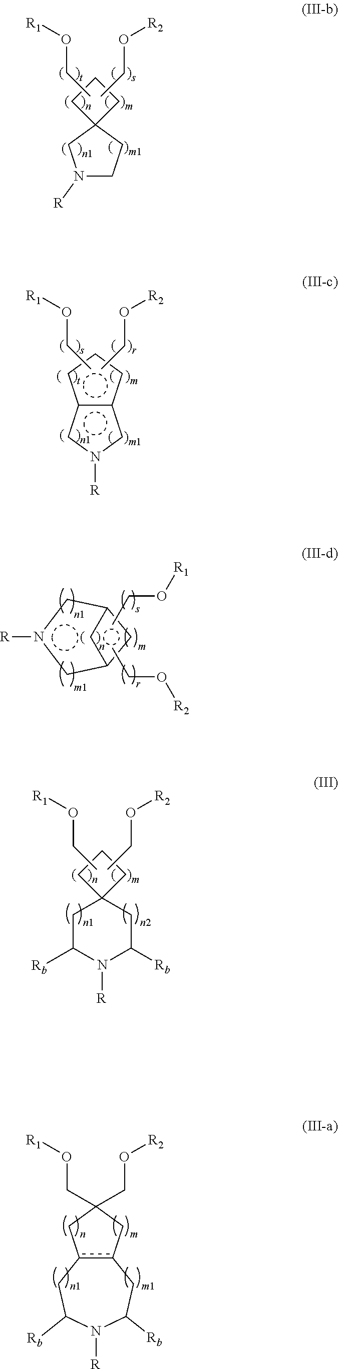
(i) in formula (III-b), (III-c) or (III-d);
R₁ is hydrogen or a protecting group;
R₂ is hydrogen or a solid support, optionally linked via a linker group, or a phosphoramidite;
R is a carbohydrate or a derivative thereof having hydroxyl protecting group, linked via a linker group;
m is independently selected from 0, 1, 2, 3; 4 and 5;
n is independently selected from 1, 2, 3; 4 and 5;
each of r, s and t is independently selected from 0, 1 and 2;
each of m1 and n1 is each independently selected from 0, 1, 2, 3 and 4;
(ii) in formula (III) or (III-a): [symbol]
is a single or double bond;
R₁ is hydrogen or a protecting group;
R₂ is hydrogen or a solid support, optionally linked via a linker group, or a phosphoramidite;
R is a carbohydrate or a derivative thereof having hydroxyl protecting group, linked via a linker group;
R_{b} is independently selected from the group of hydrogen and oxo;
each of m and n is independently selected from 1, 2, 3; 4 and 5;
each of m1 and n1 is independently selected from 0, 1, 2, 3 and 4.

2. (canceled)

3. The targeting ligand of claim 1, wherein the R having the structure shown in formula (IV), (IV-a), (IV-b) or (IV-c): 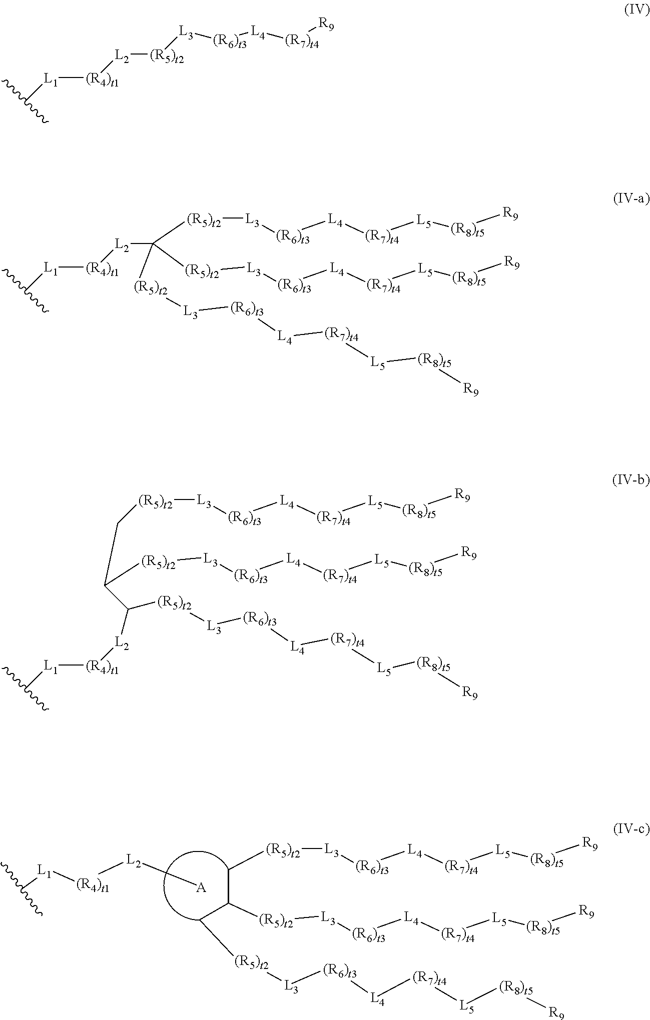
A is C₃₋₁₀ cycloalkyl or 4-12 membered heterocyclyl;
each of L₁, L₂, L₃, L₄ and L₅ is independently selected from the group of substituents consisting of absent, O, S, S—S, NH, CO, CONH, NHCO and 4-10 membered heterocyclyl;
each of R₄, R₆, R₇ and R₈ is independently selected from the group of substituents consisting of CH₂, OCH₂CH₂ and CH₂CH₂O;
R₅ is independently selected from the group of substituents consisting of absent, CH₂, OCH₂, CH₂O, OCH₂CH₂, CH₂CH₂O, NHCH₂, CH₂NH, NHCH₂CH₂, CH₂CH₂NH, C₀₋₆ alkyl (C₃₋₈ cycloalkyl) and C₀₋₆ alkyl (4-10 membered heterocyclyl);
R₉ is a carbohydrate;
each of t1, t2, t3, t4 and t5 is independently selected from 0, 1, 2, 3, 4, 5 and 6.

4. The targeting ligand of claim 3, wherein the R having the structure shown in formula (IV-1), (IV-a-1), (IV-b-1) or (IV-c-1): 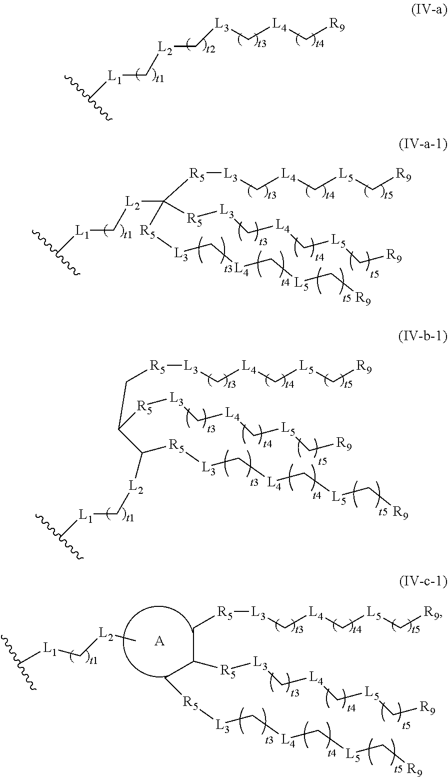
wherein,
A is 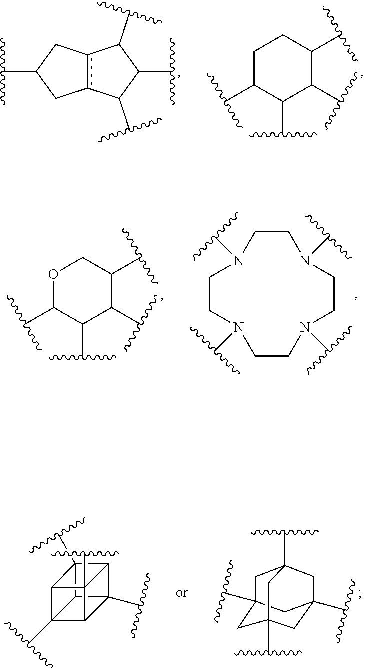
each of L₁, L₃, L₄ and L₅ is independently selected from the group of substituents consisting of absent, O, S, S—S, NH, CO, CONH and NHCO;
L₂ is absent, O, S, S—S, NH, CO, CONH, NHCO and 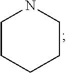
R₅ is is independently selected from the group of substituents consisting of absent, CH₂, CH₂O, CH₂NH, C₃₋₆ cycloalkyl, C₃₋₆ CycloalkylC₁₋₃ alkyl, 4-8 membered heterocyclyl containing 1, 2 or 3 of heteroatoms selected from N or O and 4-8 membered heterocyclylC₁₋₃ alkyl containing 1, 2 or 3 of ring heteroatoms selected from N or O.

5. The targeting ligand of claim 3, wherein the R₅ is independently selected from —OCH₂—, —NHCH₂—,

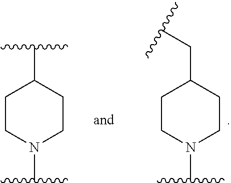 [SMILES: *C1CCN(*)CC1 ; *CC1CCN(*)CC1]

6. The targeting ligand of claim 3, wherein R₉ is selected from the group consisting of galactose, galactosamine, N-acetylgalactosamine (GalNAc), D-galactosaminitol, mannose, mannosamine, mannose-6-phosphate, glucose, glucosamine, N-acetyl-glucosamine (GluNAc), glucose-6-phosphate, glucosaminitol, glucose glyceraldehyde, fucose, fucosamine, fuculose, lactose, allose, altrose, arabinose, cladinose, erythrose, erythrulose, fructose, D-fucitol, L-fucitol, L-glycero-D-mannos-heptose, glycerol, glycerone, gulose, idose, lyxose, psicose, quinovose, quinovosamine, rhamnose, rhamnitol, rhamnosamine, ribose, ribulose, sedoheptulose, sorbose, tagatose, talose, tartaric acid, threose, xylose, and xylulose; preferably, R₉ is N-acetylgalactosamine.

7. The targeting ligand of claim 3, wherein the R having the structure shown in formula (IV-d), (IV-e), (IV-f), (IV-d-a), (IV-e-a) or (IV-f-a): 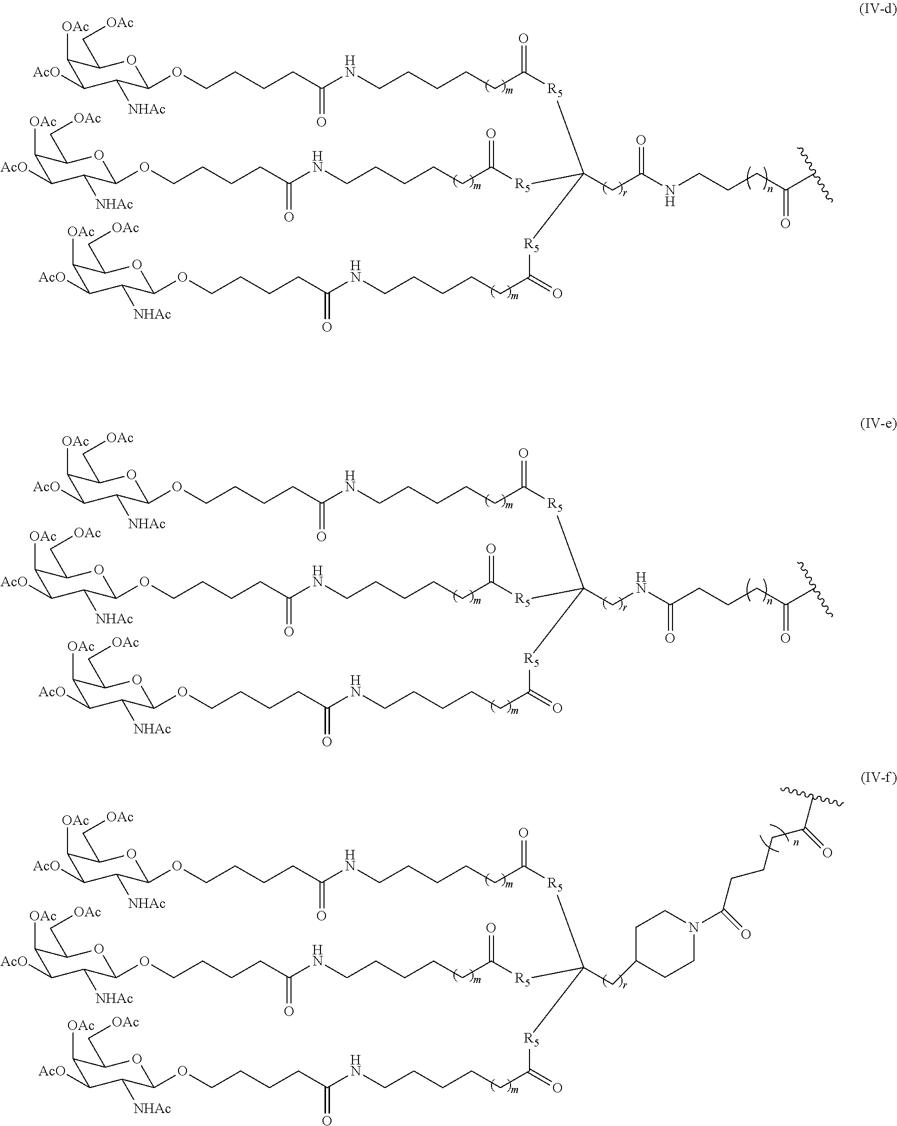 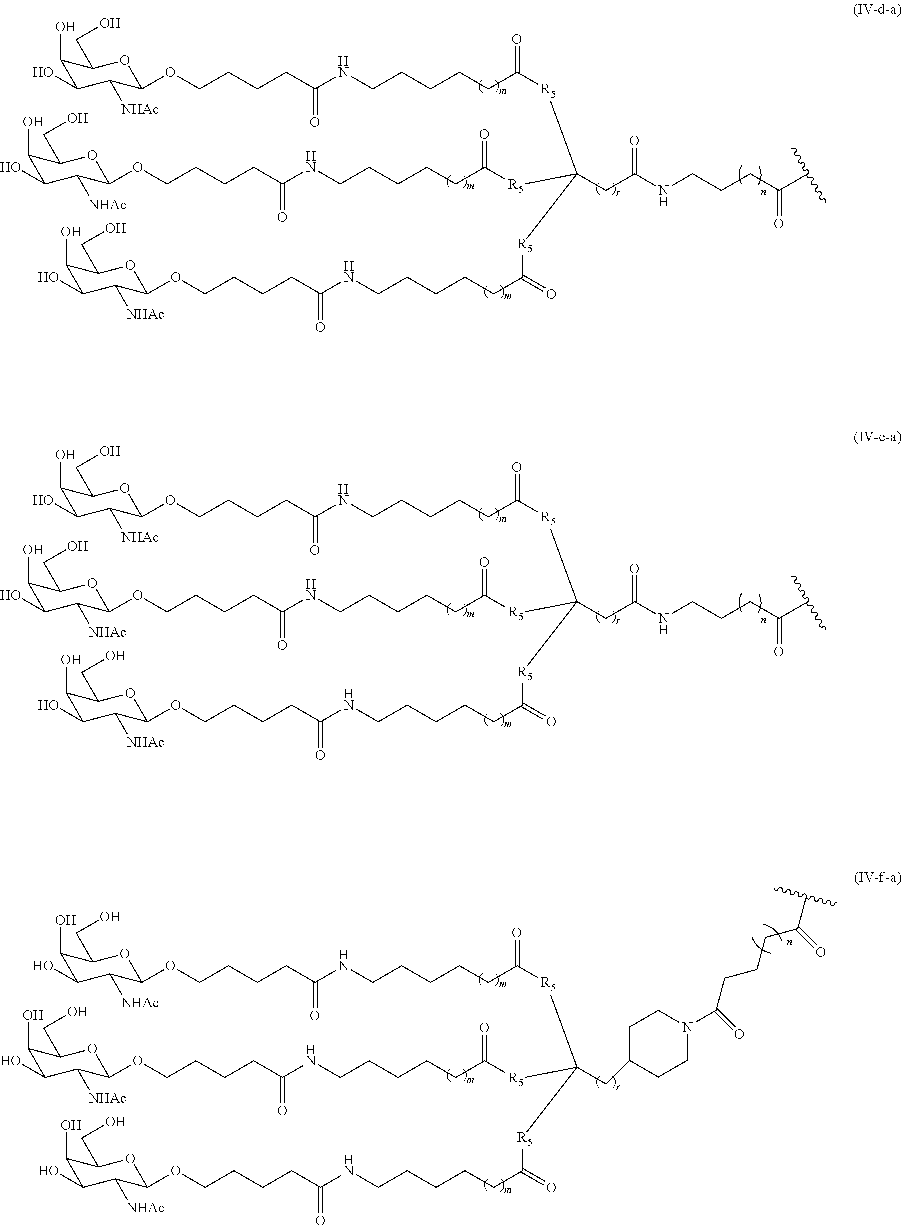
wherein,
R₅ is independently selected from the group of substituents consisting of OCH₂, NHCH₂ and 4-6 membered heterocyclyl containing 1 or 2 of heteroatoms selected from N or O;
m is independently selected from 0, 1, 2, 3, 4, 5 and 6;
n is independently selected from 1, 2, 3, 4, 5, 6, 7, 8, 9 and 10; and
r is independently selected from 0, 1, 2, 3, 4 and 5.

8. The targeting ligand of claim 3, wherein the R is: 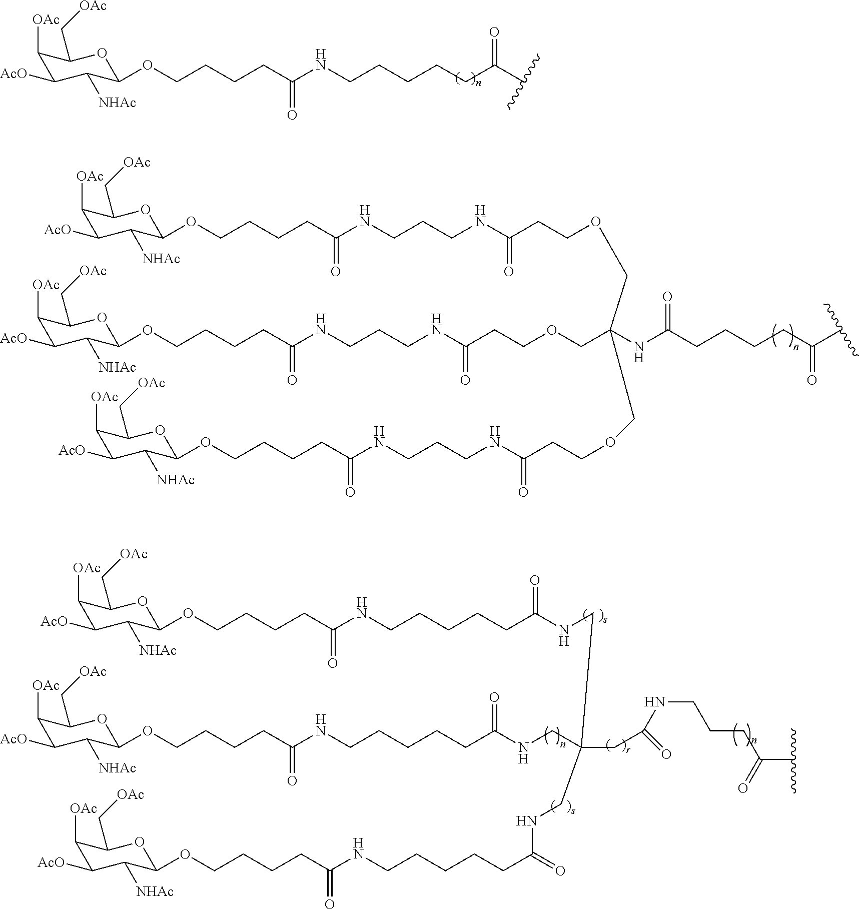 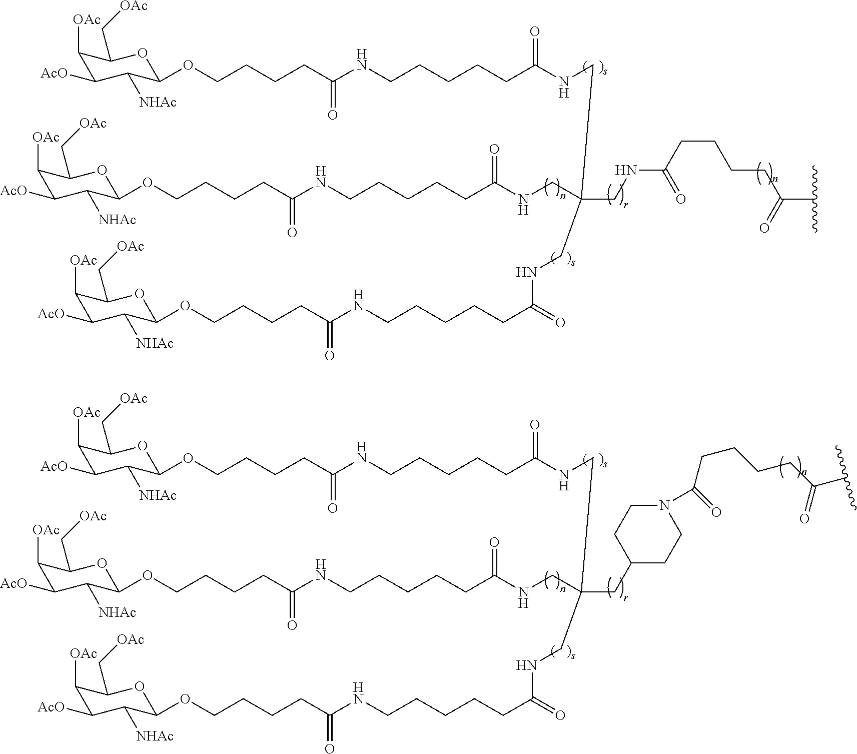 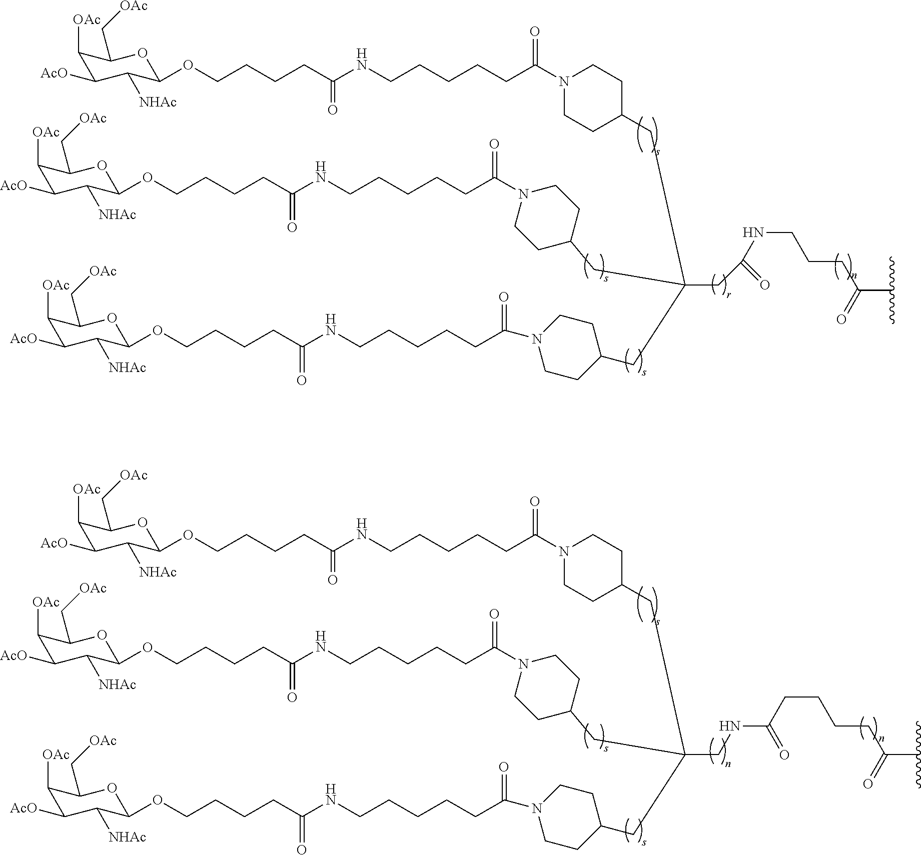 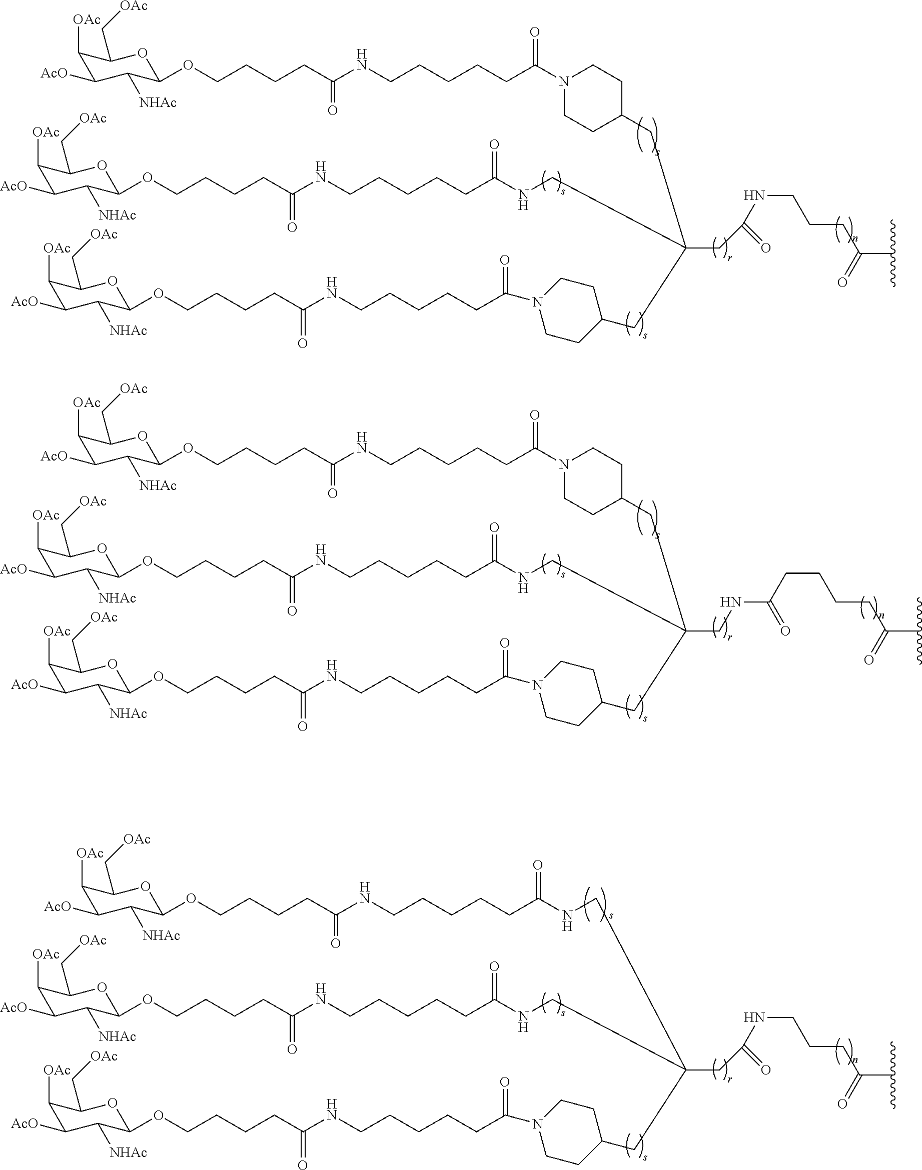 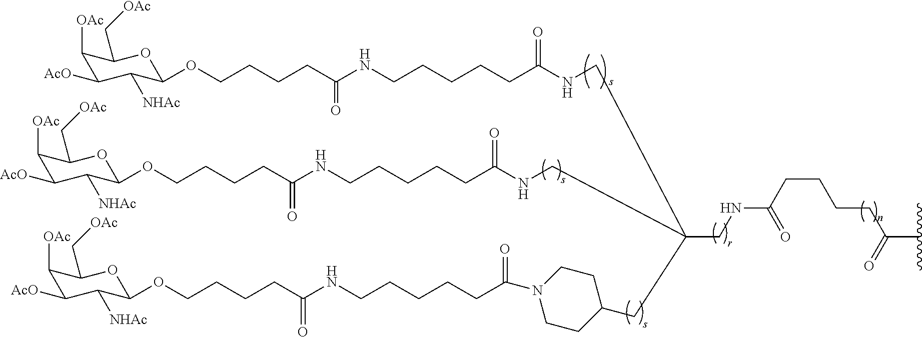
s is independently selected from 0, 1, 2 and 3;
r is independently selected from 0, 1, 2, 3, 4 and 5;
n is independently selected from 1, 2, 3, 4, 5, 6, 7, 8, 9 and 10.

9. The targeting ligand of claim 3, wherein the targeting ligand is: 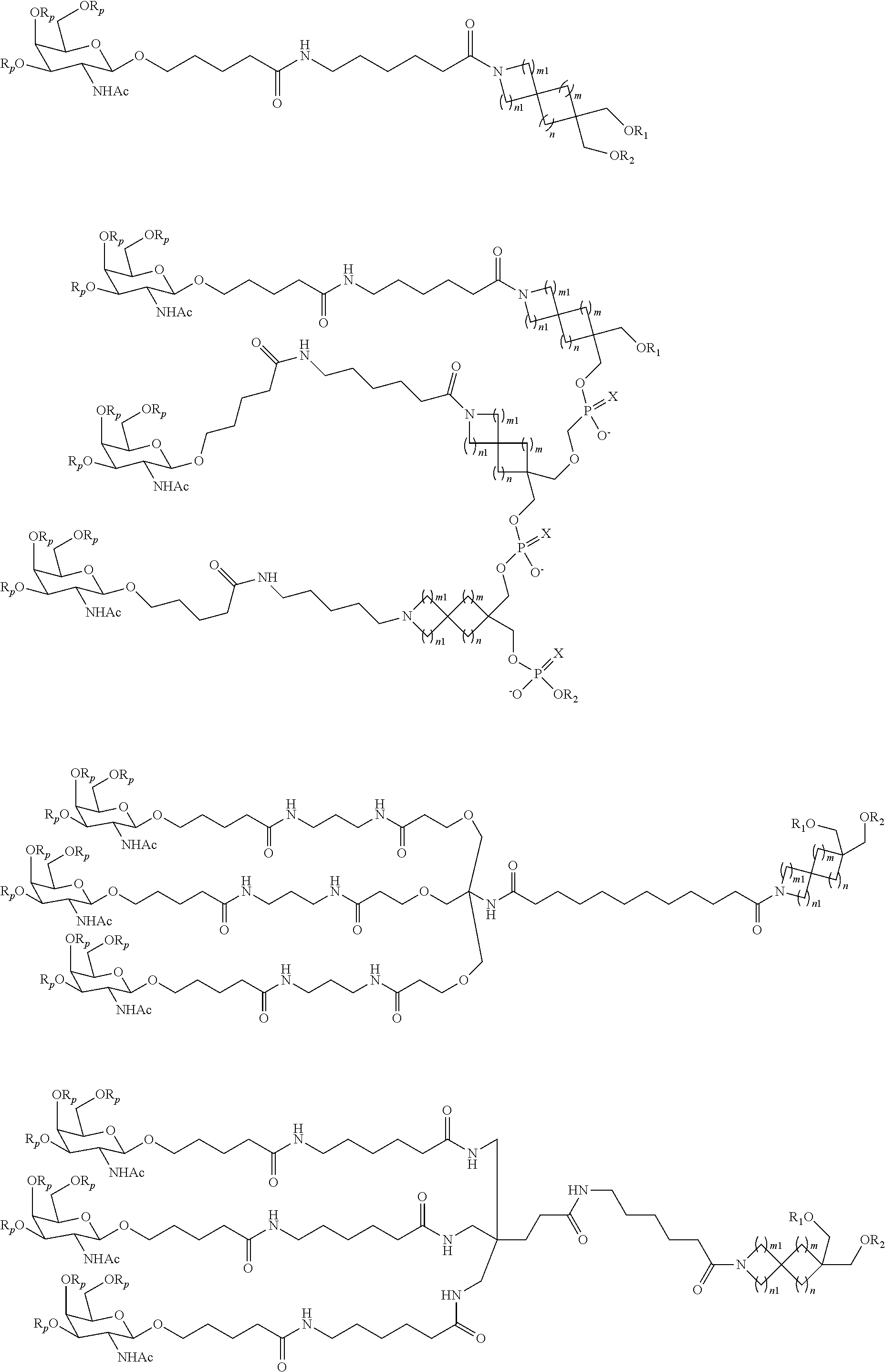 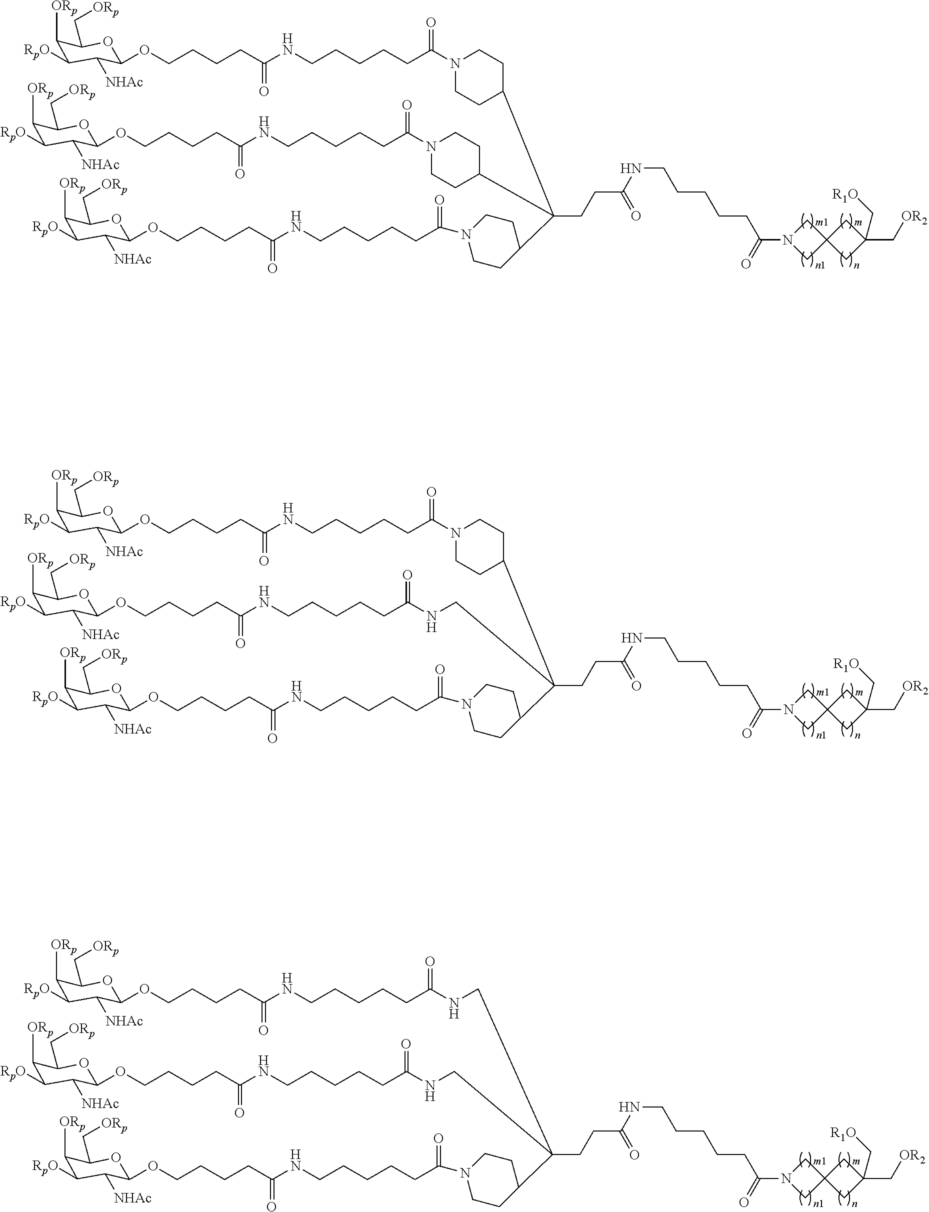 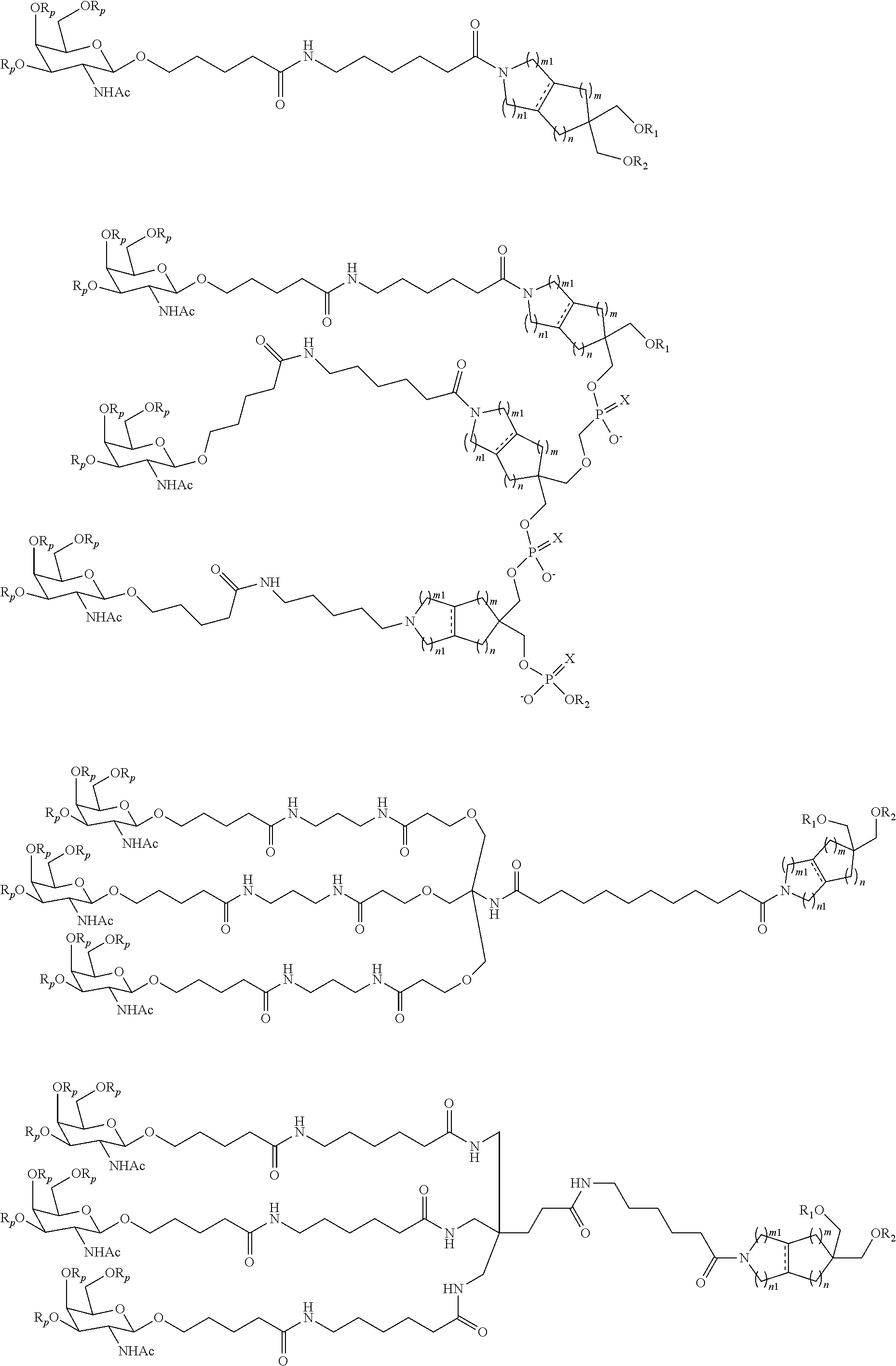 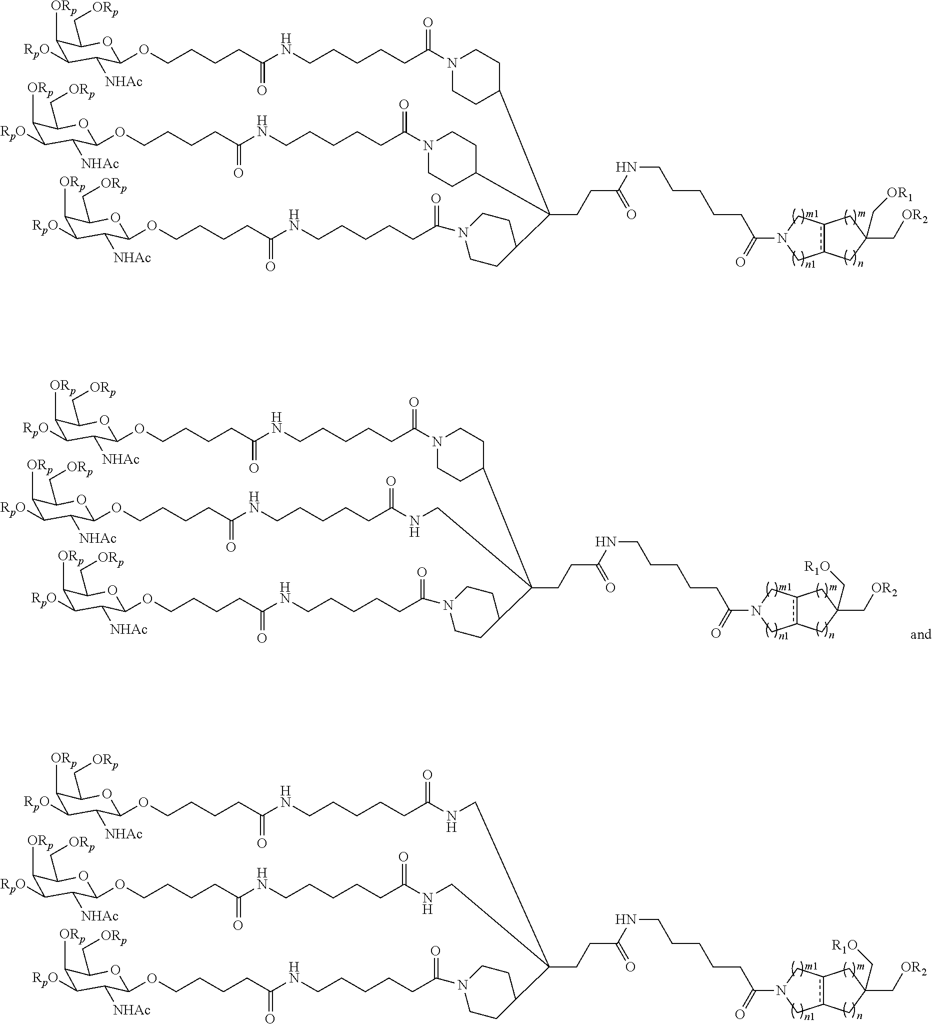
Rₚ is H or hydroxyl protecting group, preferably, hydroxyl protecting group is acetyl;
X is O or S;
each of m and n is independently selected from 1 or 2;
each of m1 and n1 is independently selected from 1, 2 or 3.

10. The targeting ligand of claim 3, wherein R₁ is hydrogen or substituted or unsubstituted triphenylmethyl hydroxyl protecting group; preferably, R₁ is

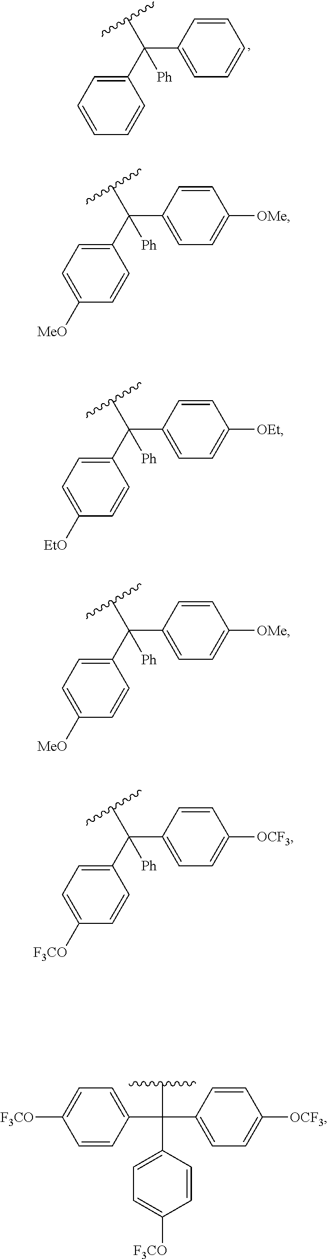 [SMILES: *C(c1ccccc1)(c1ccccc1)c1ccccc1 ; *C(c1ccccc1)(c1ccc(OC)cc1)c1ccc(OC)cc1 ; *C(c1ccccc1)(c1ccc(OCC)cc1)c1ccc(OCC)cc1 ; *C(c1ccccc1)(c1ccc(OC)cc1)c1ccc(OC)cc1 ; *C(c1ccccc1)(c1ccc(OC(F)(F)F)cc1)c1ccc(OC(F)(F)F)cc1 ; *C(c1ccc(OC(F)(F)F)cc1)(c1ccc(OC(F)(F)F)cc1)c1ccc(OC(F)(F)F)cc1]

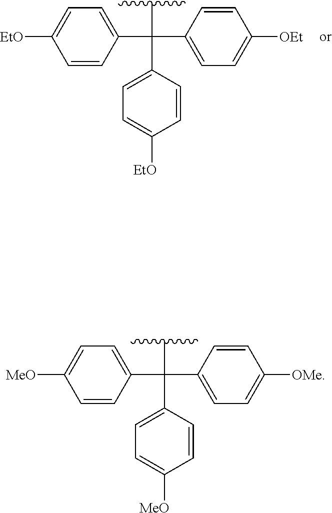 [SMILES: *C(c1ccc(OCC)cc1)(c1ccc(OCC)cc1)c1ccc(OCC)cc1 ; *C(c1ccc(OC)cc1)(c1ccc(OC)cc1)c1ccc(OC)cc1]

11. The targeting ligand of claim 3, wherein R₂ is hydrogen, 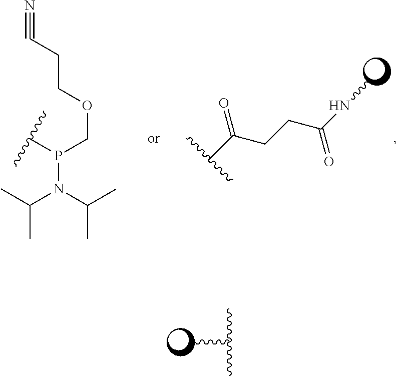
is a solid support.

12. The targeting ligand of claim 3, wherein the targeting ligand is: 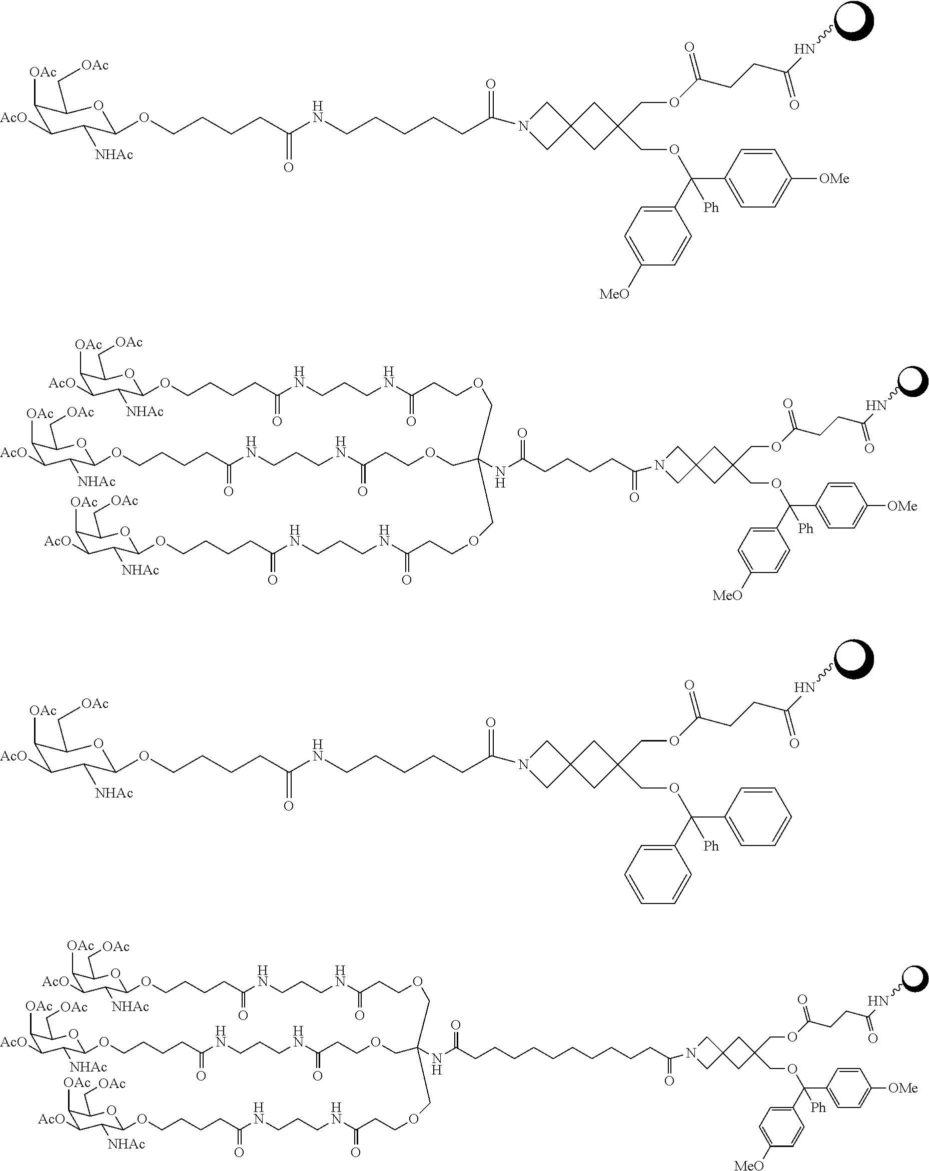 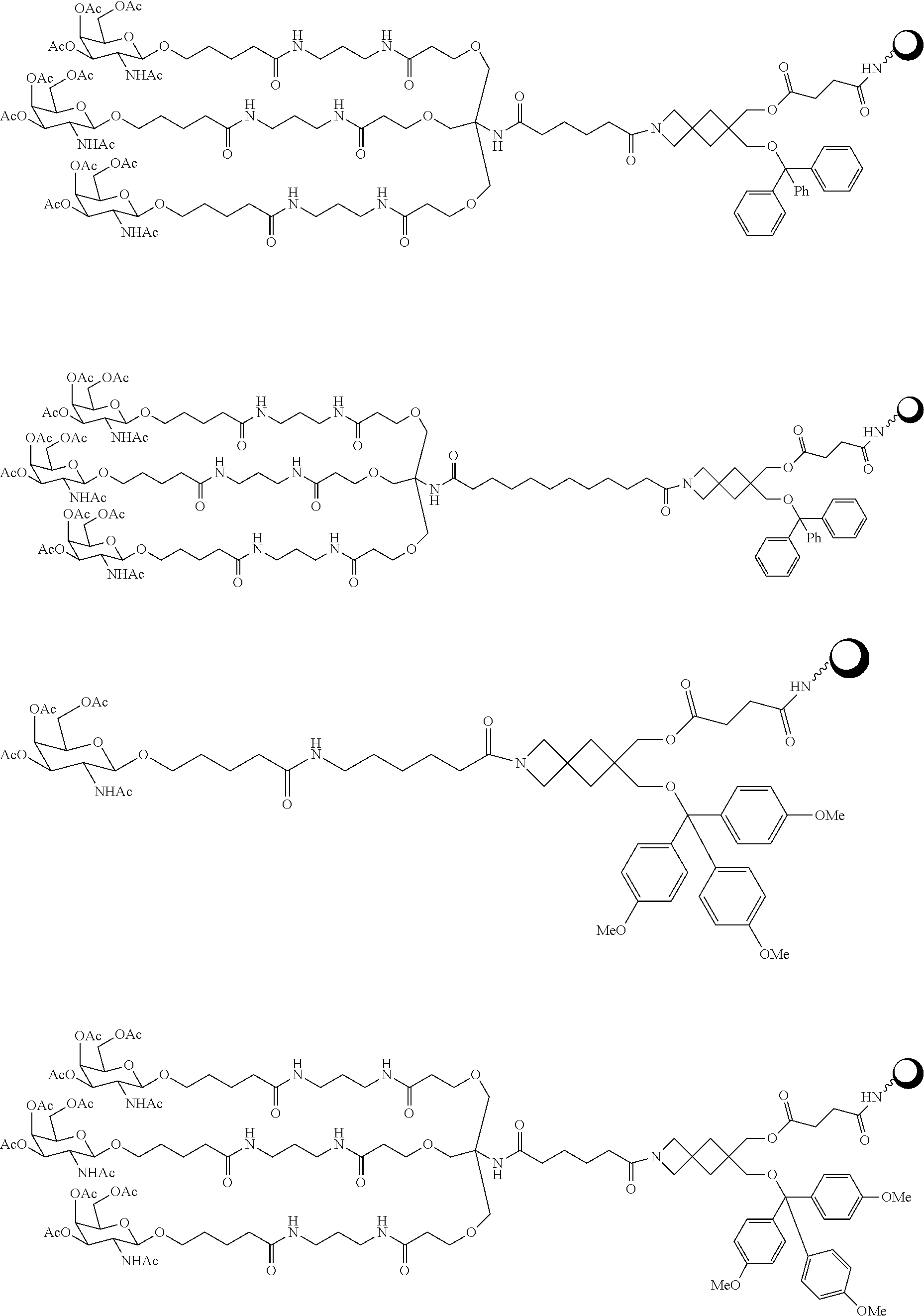 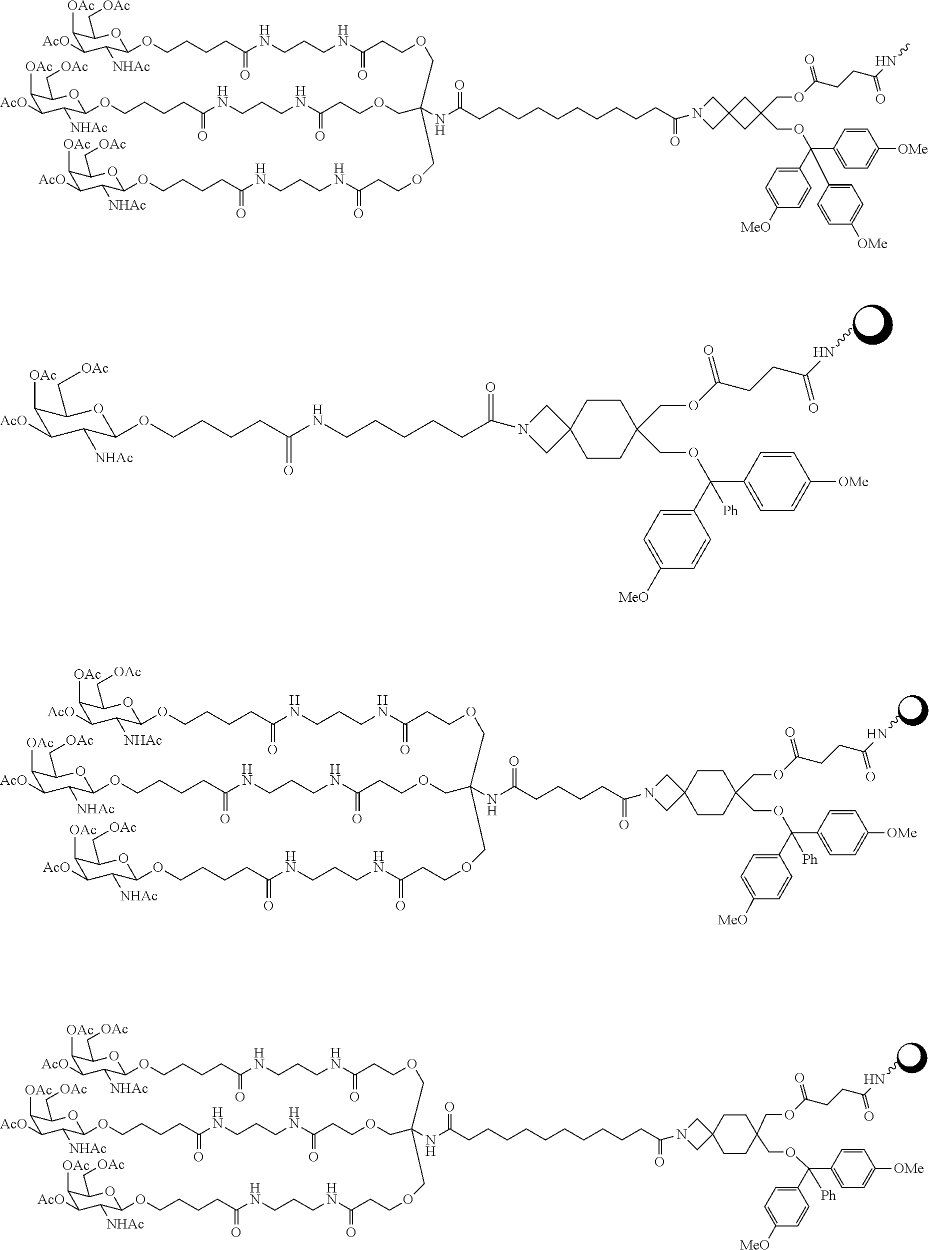 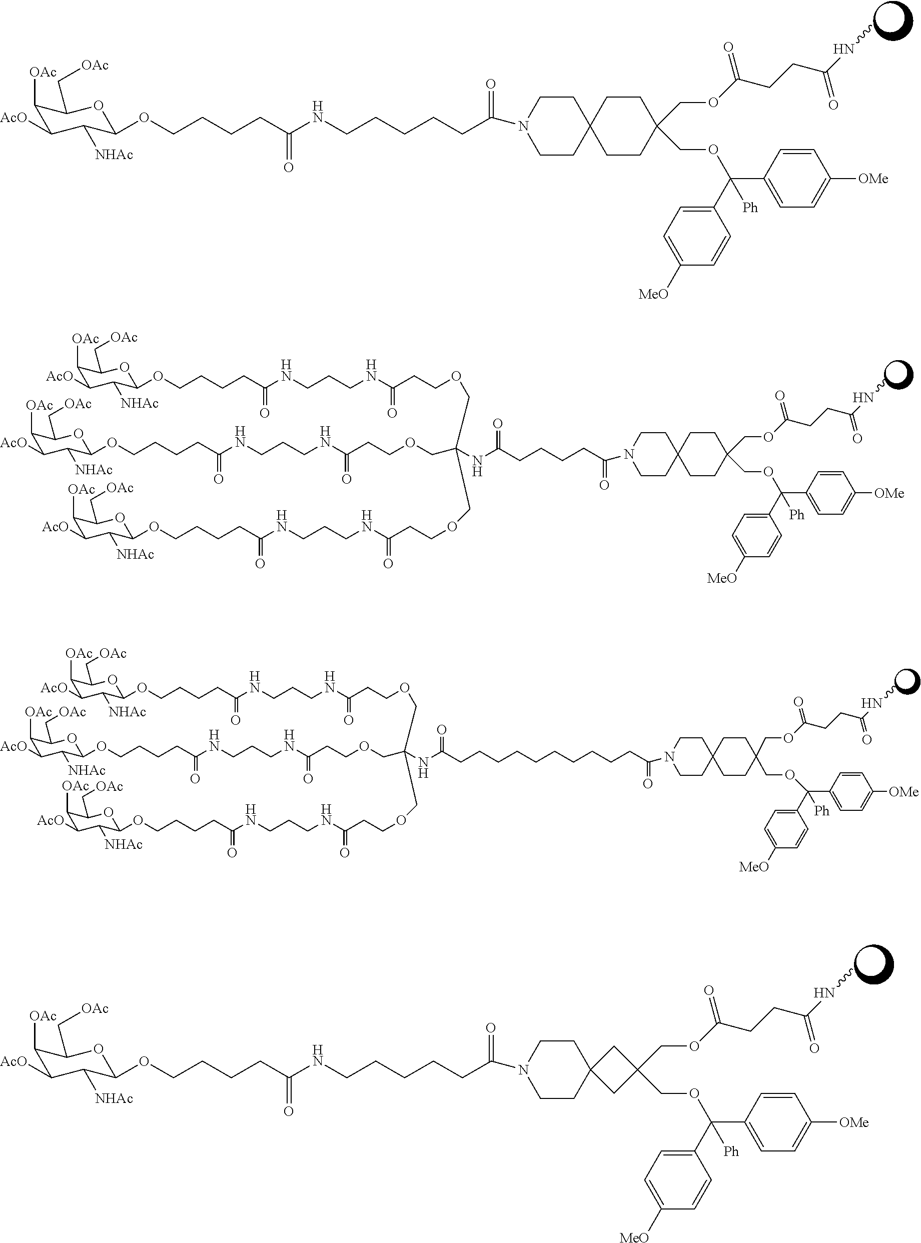 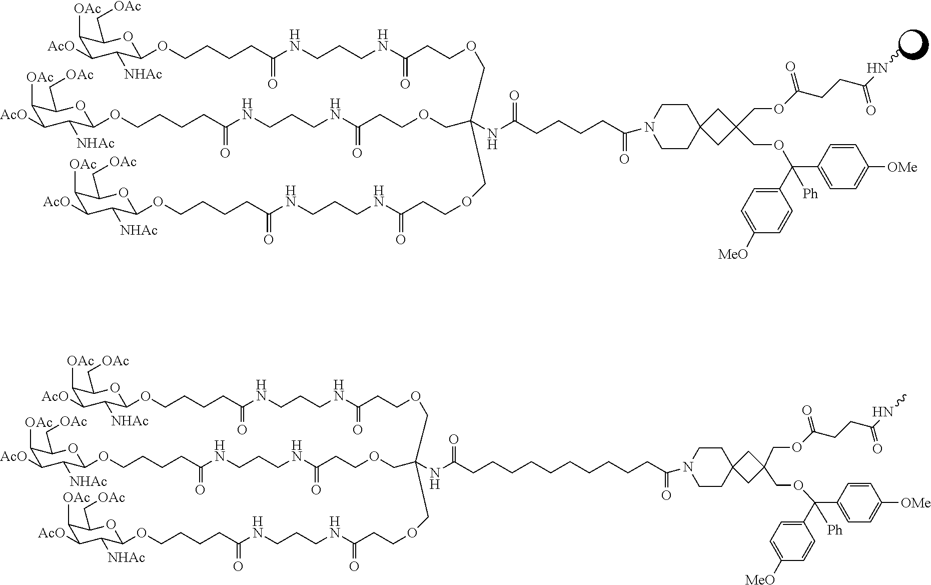 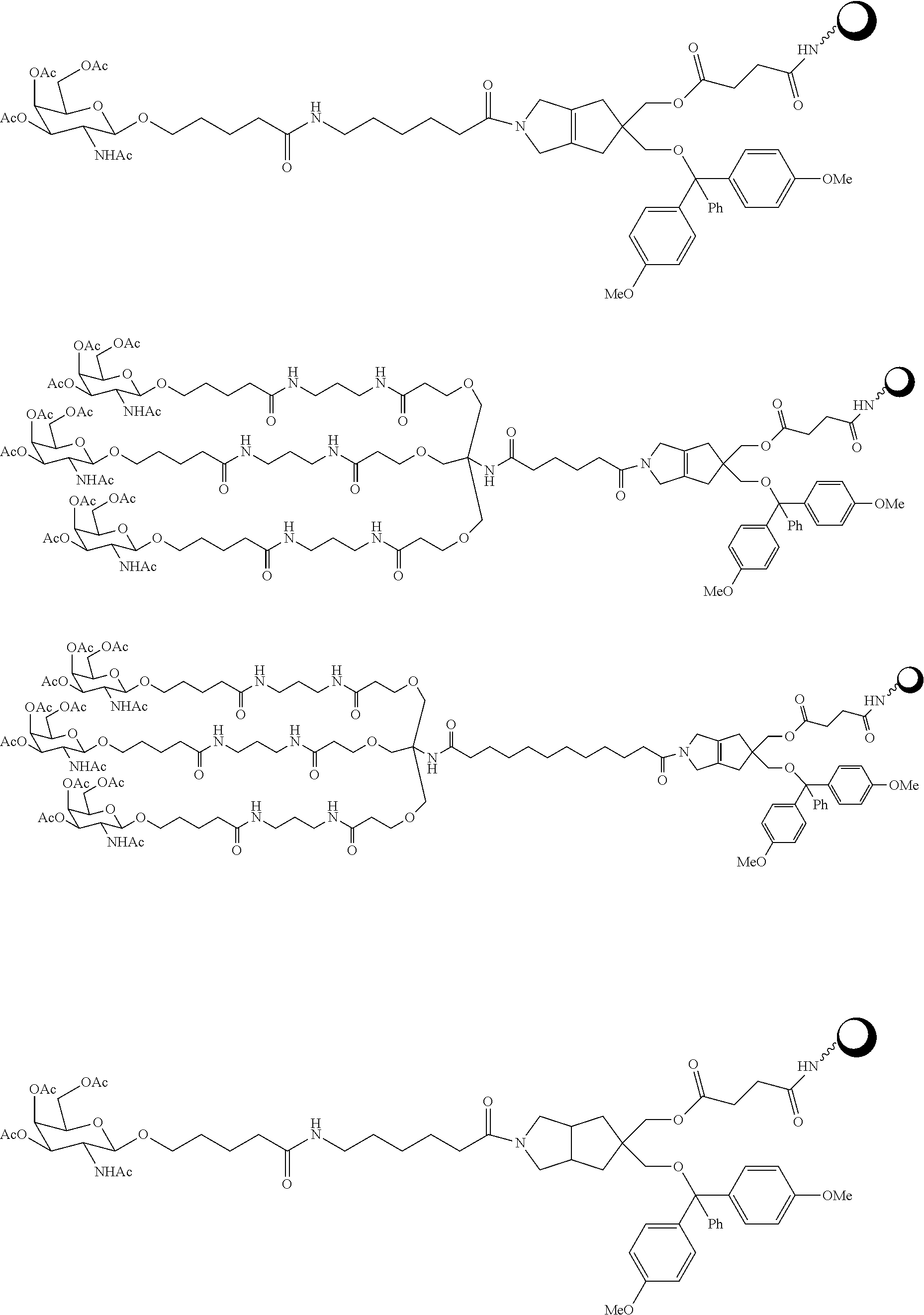 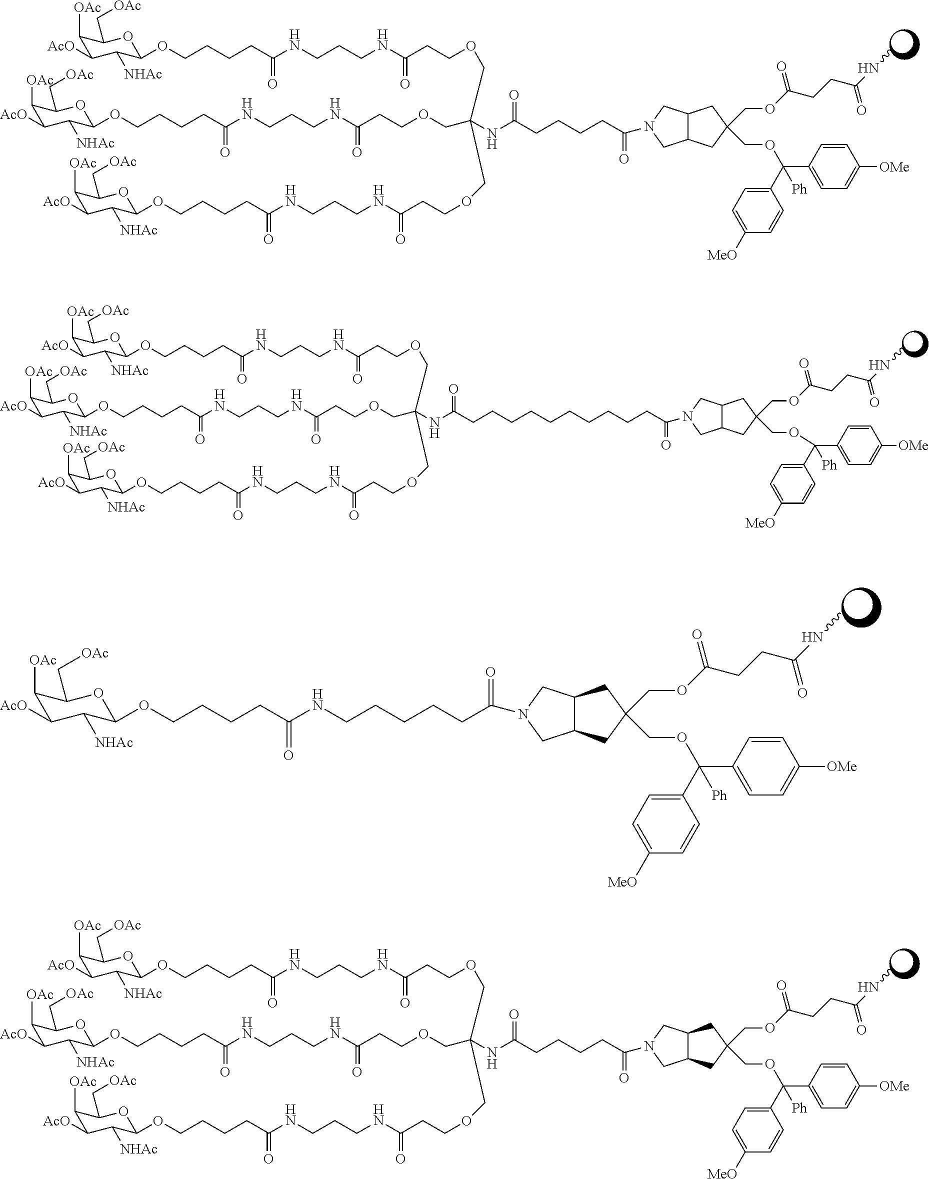 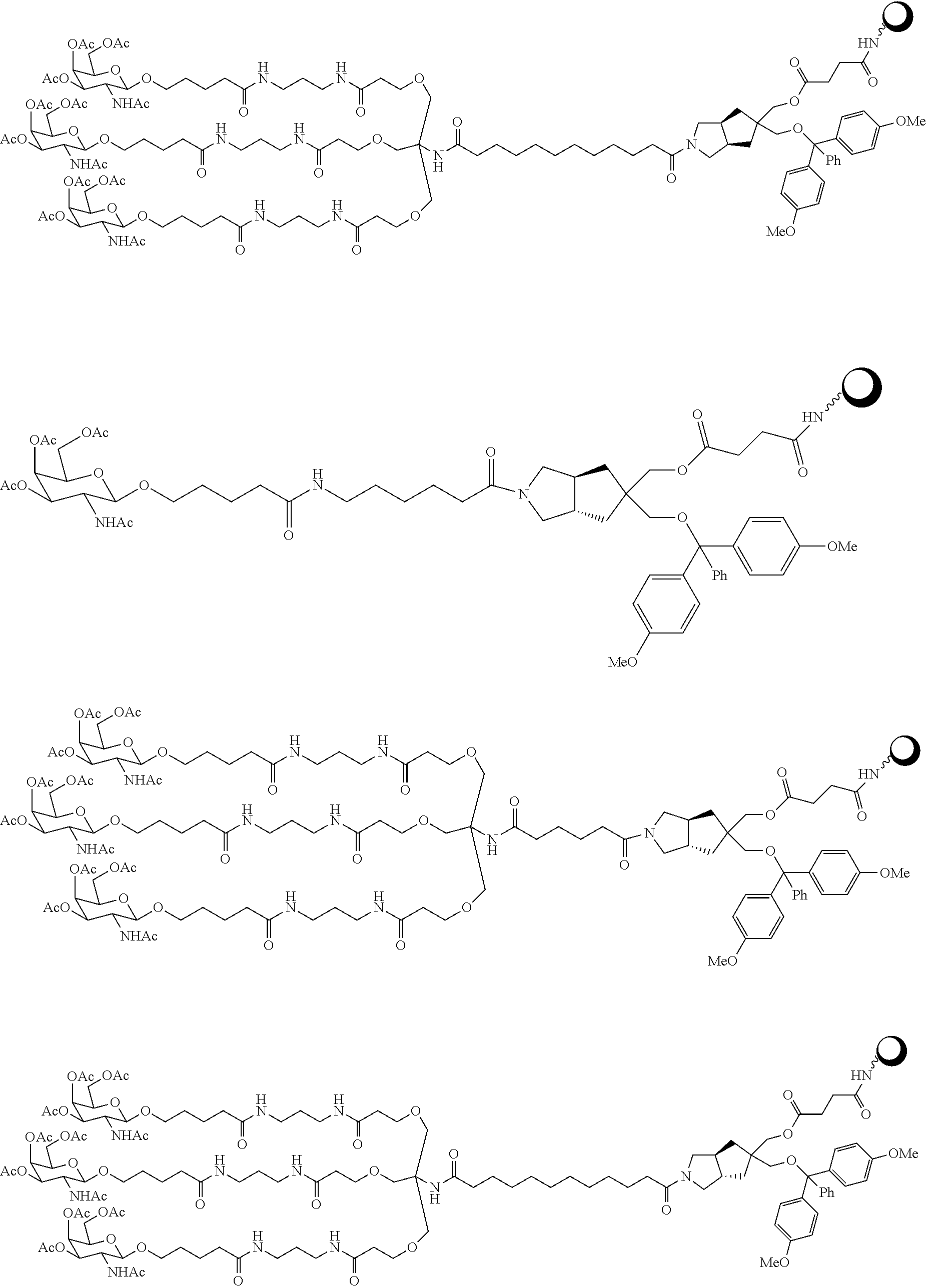 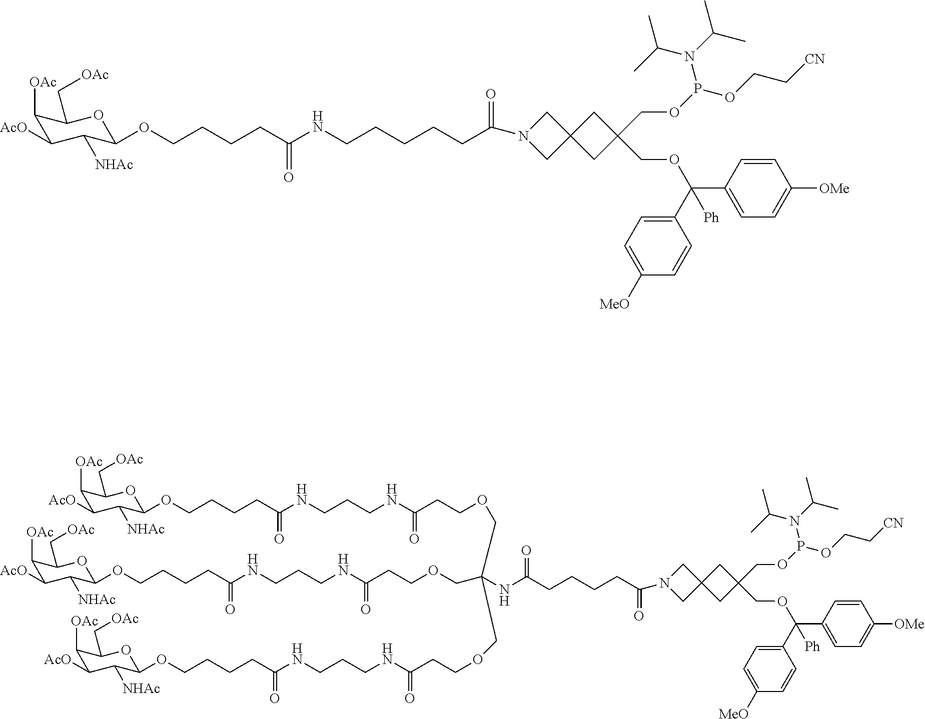 [SMILES: COc1ccc(C(OCC2(COP(OCCC#N)N(C(C)C)C(C)C)CC3(CN(C(=O)CCCCCNC(=O)CCCCOC4OC(COC(C)=O)C(OC(C)=O)C(OC(C)=O)C4NC(C)=O)C3)C2)(c2ccccc2)c2ccc(OC)cc2)cc1 ; COc1ccc(C(OCC2(COP(OCCC#N)N(C(C)C)C(C)C)CC3(CN(C(=O)CCCCC(=O)NC(COCCC(=O)NCCCNC(=O)CCCCOC4OC(COC(C)=O)C(OC(C)=O)C(OC(C)=O)C4NC(C)=O)(COCCC(=O)NCCCNC(=O)CCCCOC4OC(COC(C)=O)C(OC(C)=O)C(OC(C)=O)C4NC(C)=O)COCCC(=O)NCCCNC(=O)CCCCOC4OC(COC(C)=O)C(OC(C)=O)C(OC(C)=O)C4NC(C)=O)C3)C2)(c2ccccc2)c2ccc(OC)cc2)cc1] 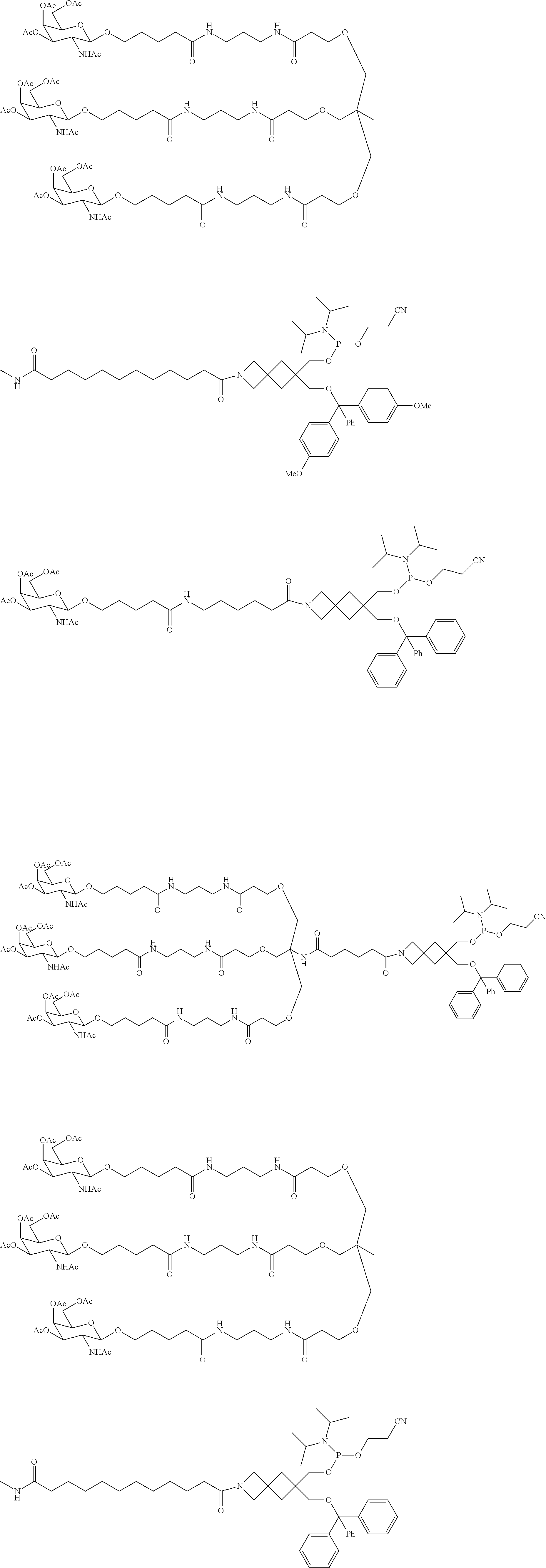 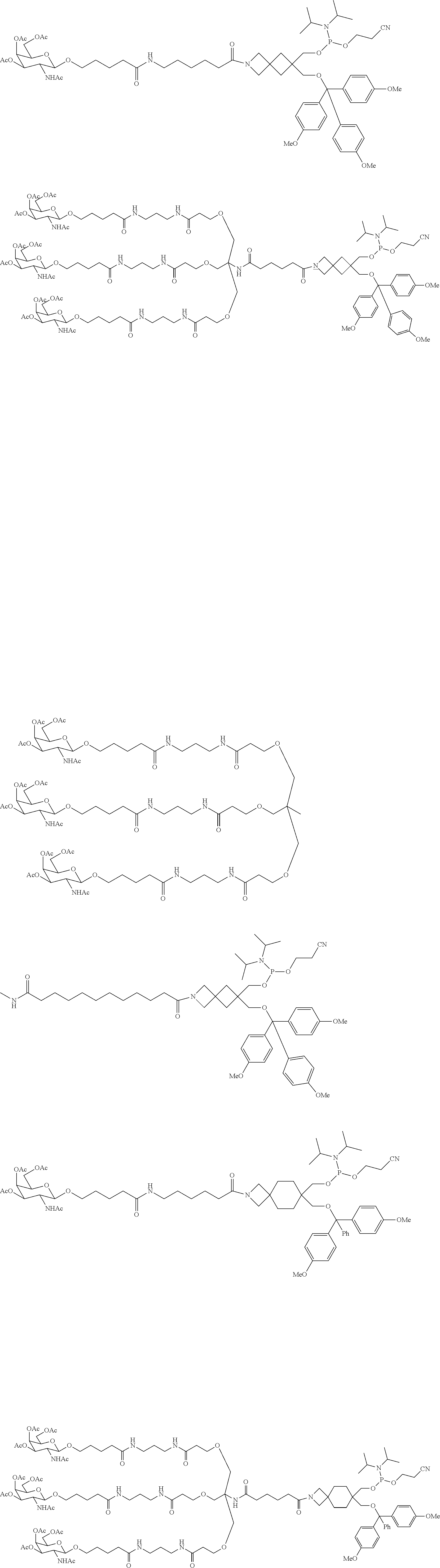 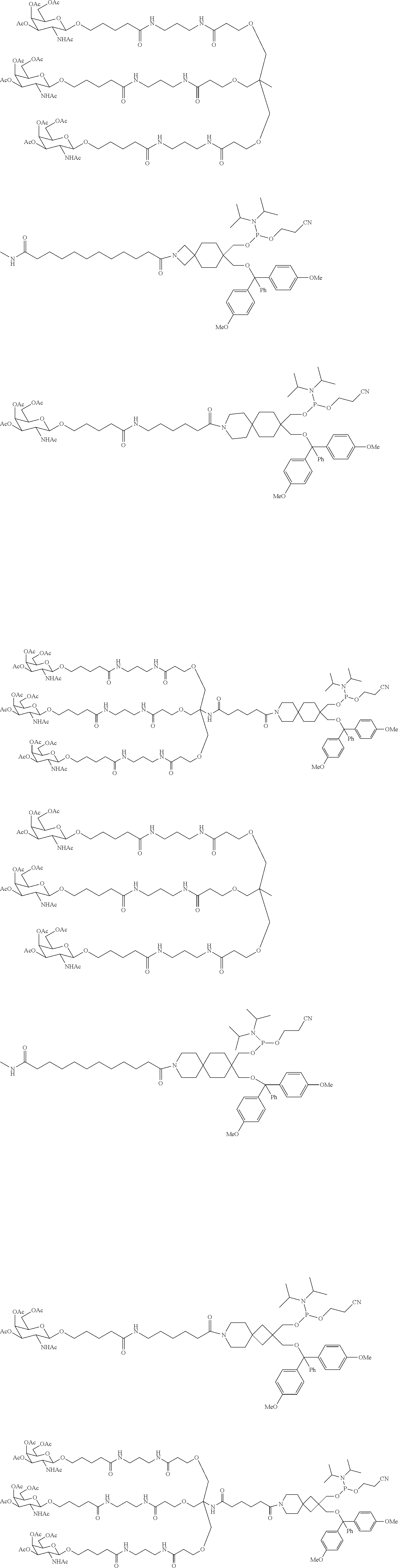 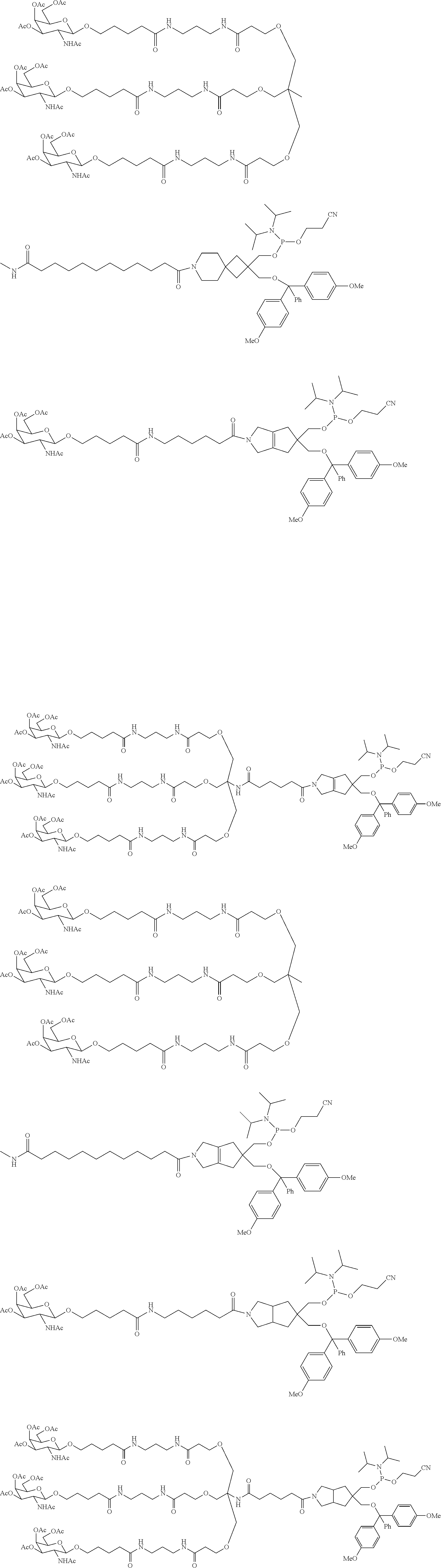 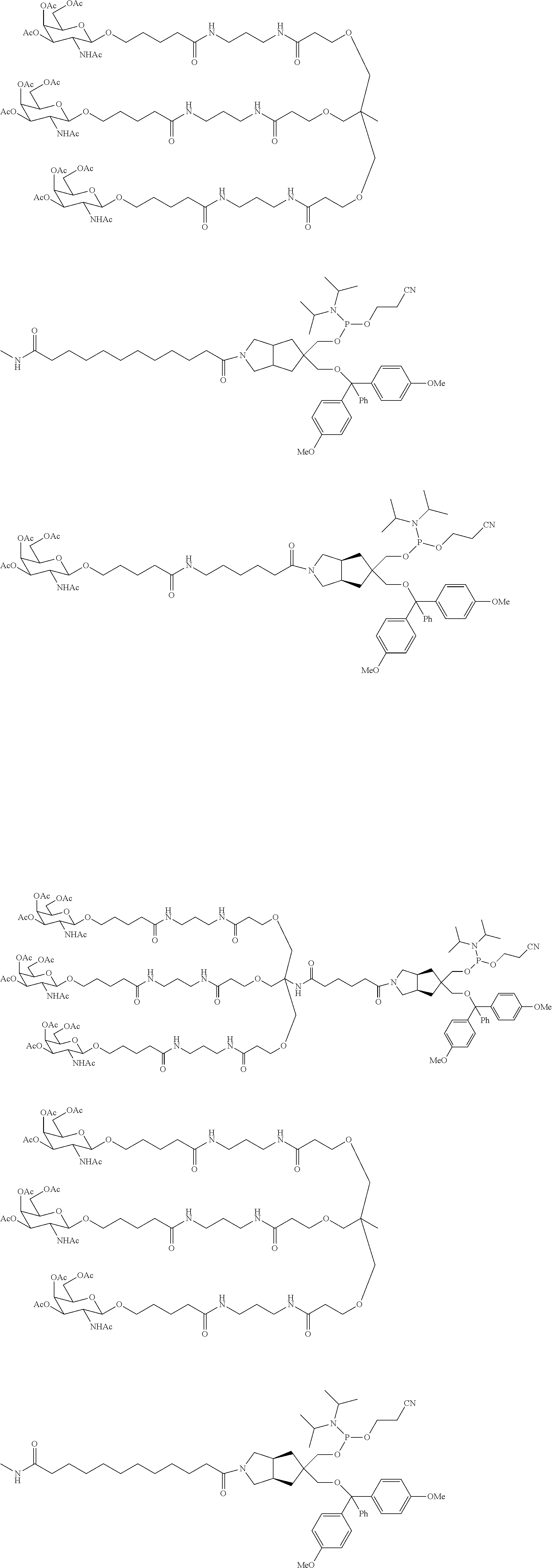 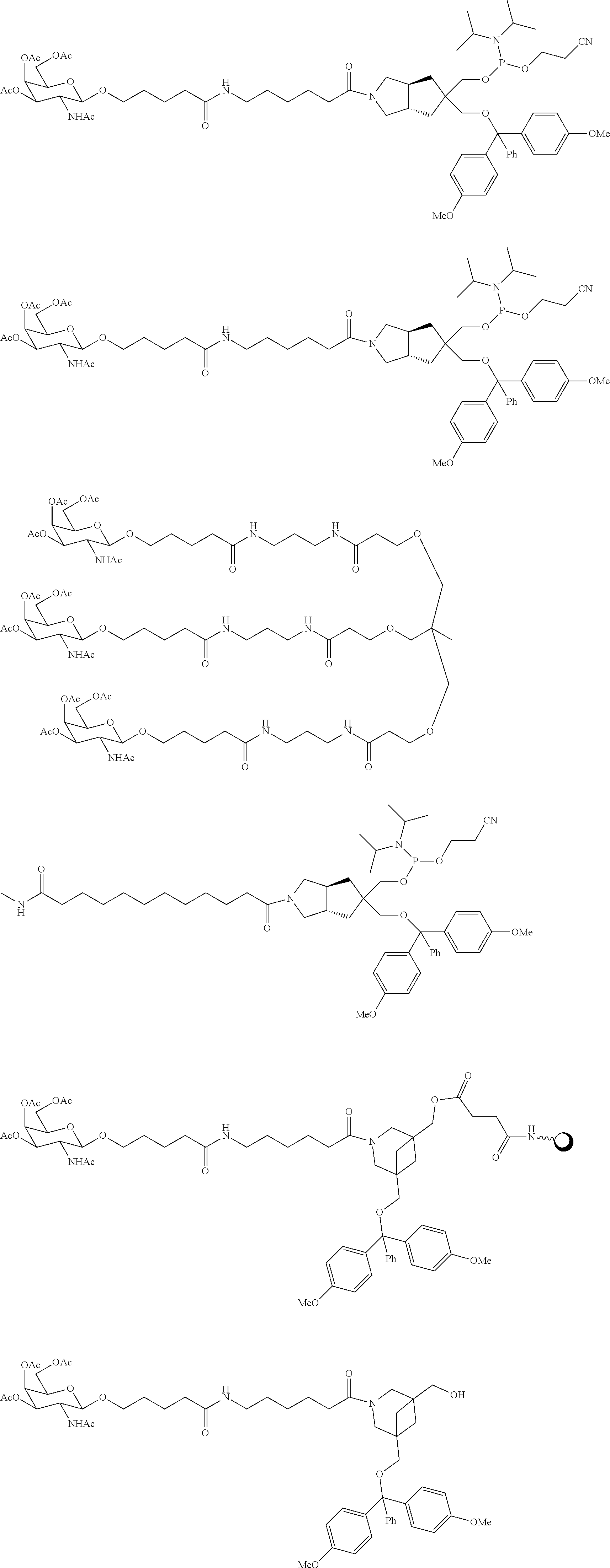 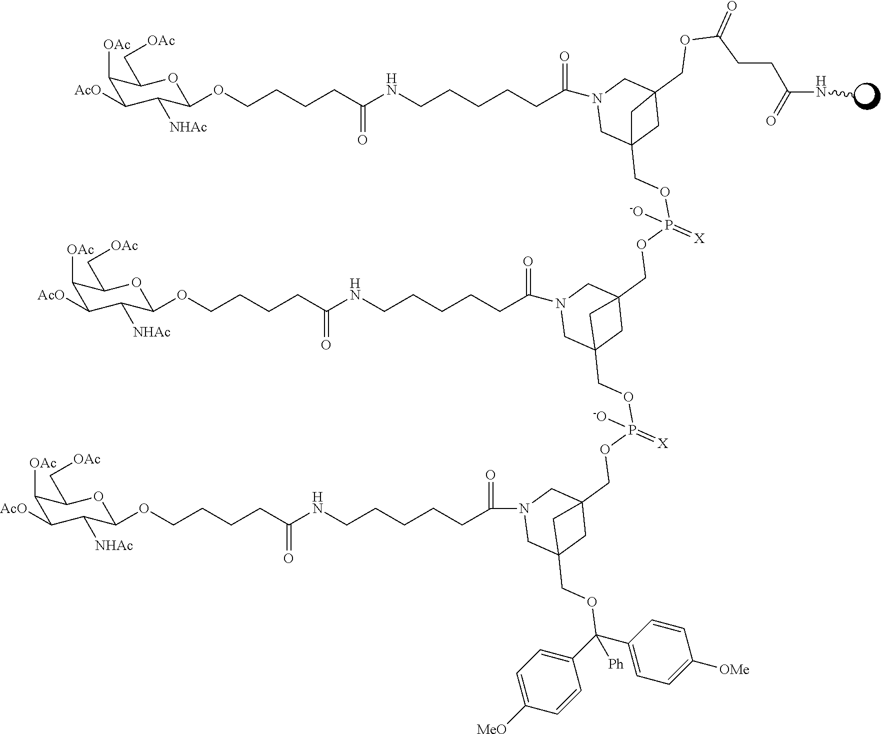 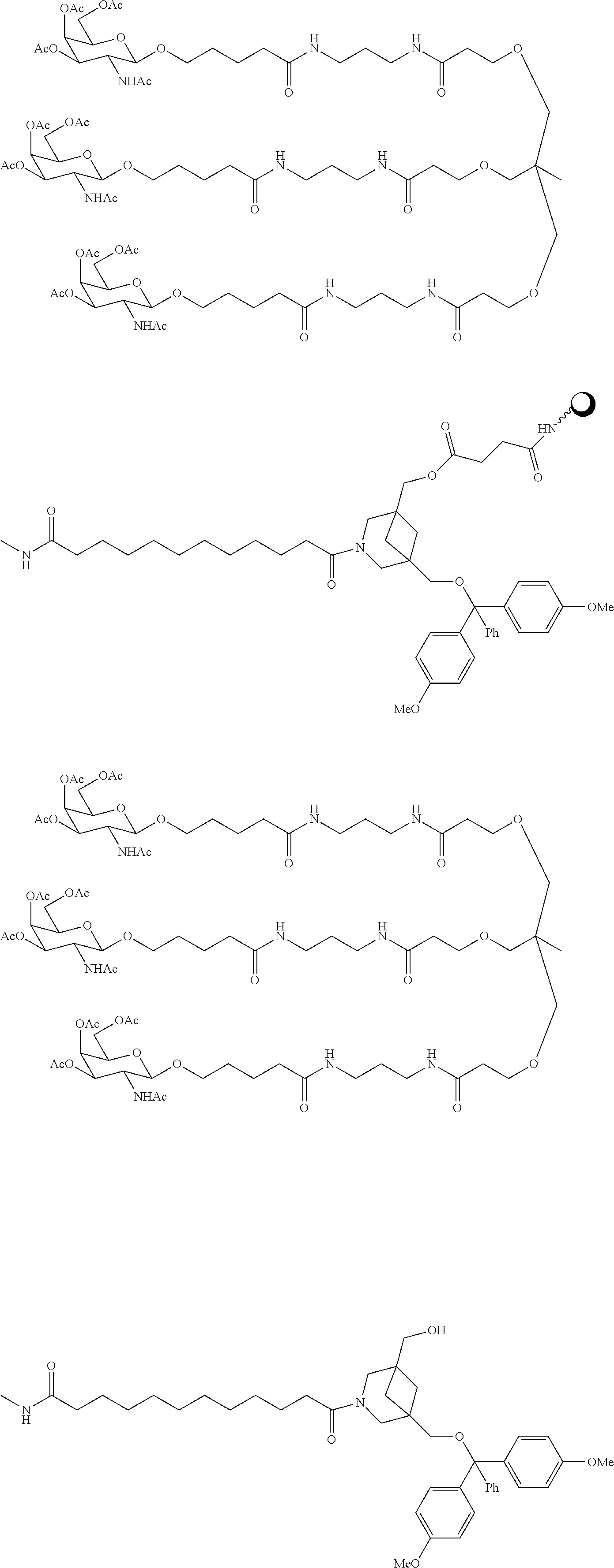 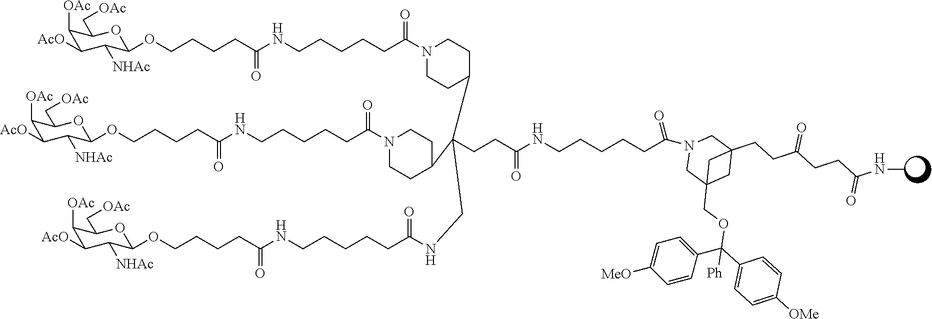 [SMILES: CNC(=O)CCC(=O)CCC12CN(C(=O)CCCCCNC(=O)CCC(CNC(=O)CCCCCNC(=O)CCCCOC3OC(COC(C)=O)C(OC(C)=O)C(OC(C)=O)C3NC(C)=O)(C3CCN(C(=O)CCCCCNC(=O)CCCCOC4OC(COC(C)=O)C(OC(C)=O)C(OC(C)=O)C4NC(C)=O)CC3)C3CCN(C(=O)CCCCCNC(=O)CCCCOC4OC(COC(C)=O)C(OC(C)=O)C(OC(C)=O)C4NC(C)=O)CC3)CC(COC(c3ccccc3)(c3ccc(OC)cc3)c3ccc(OC)cc3)(C1)C2] 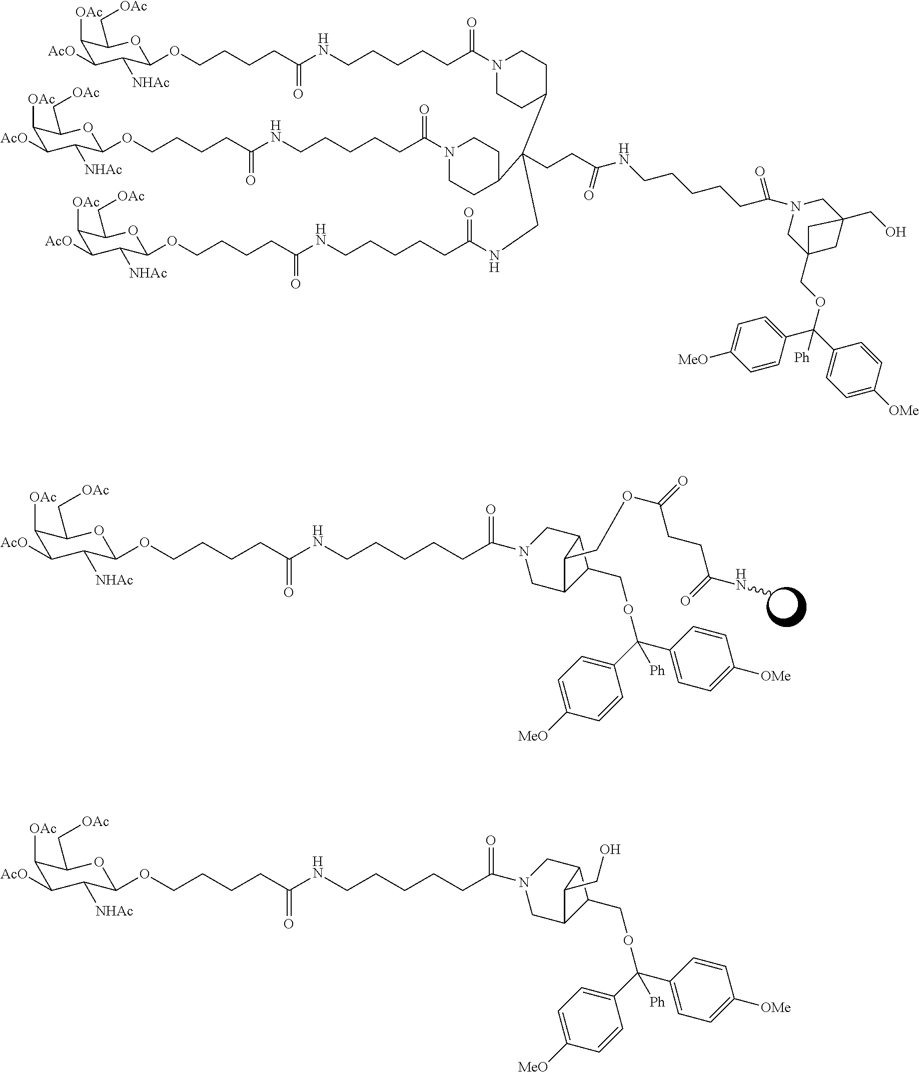 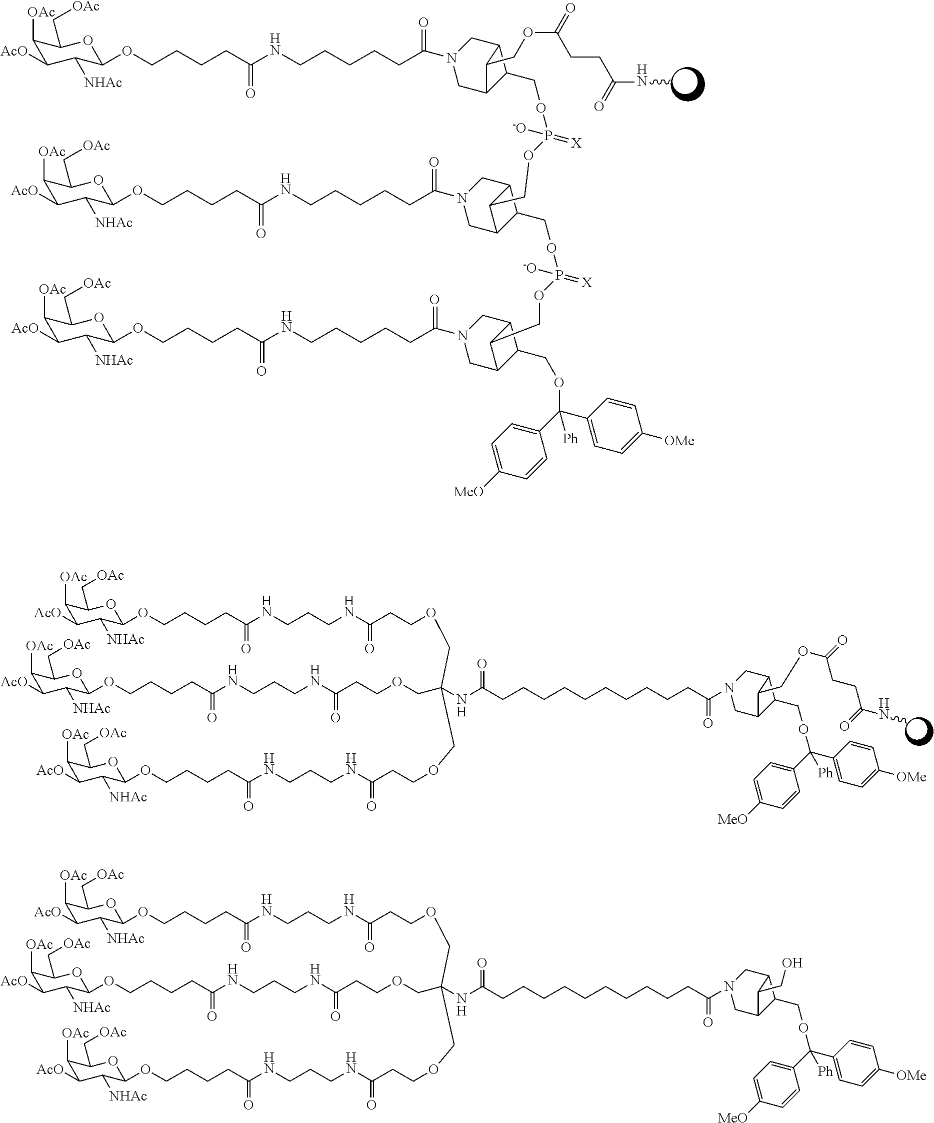 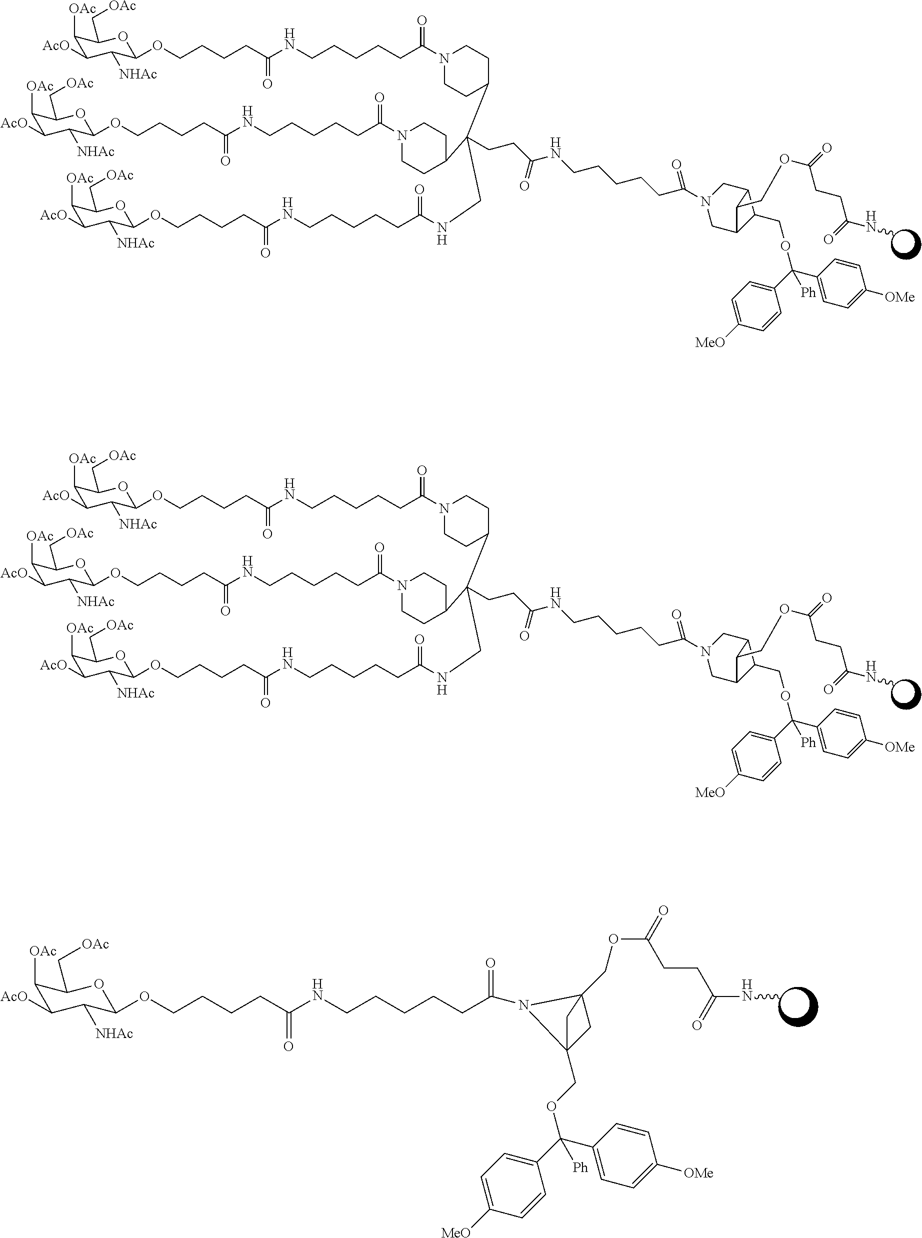 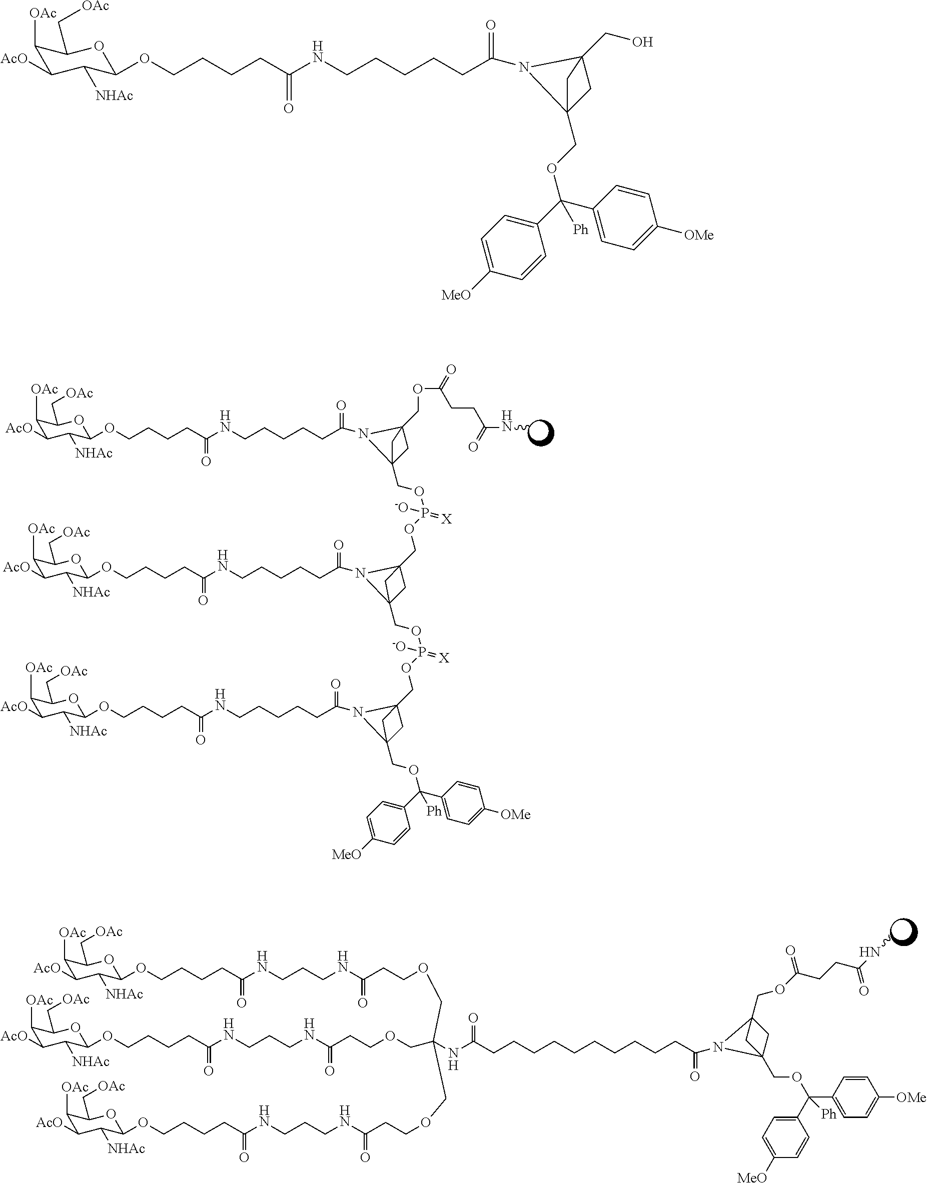 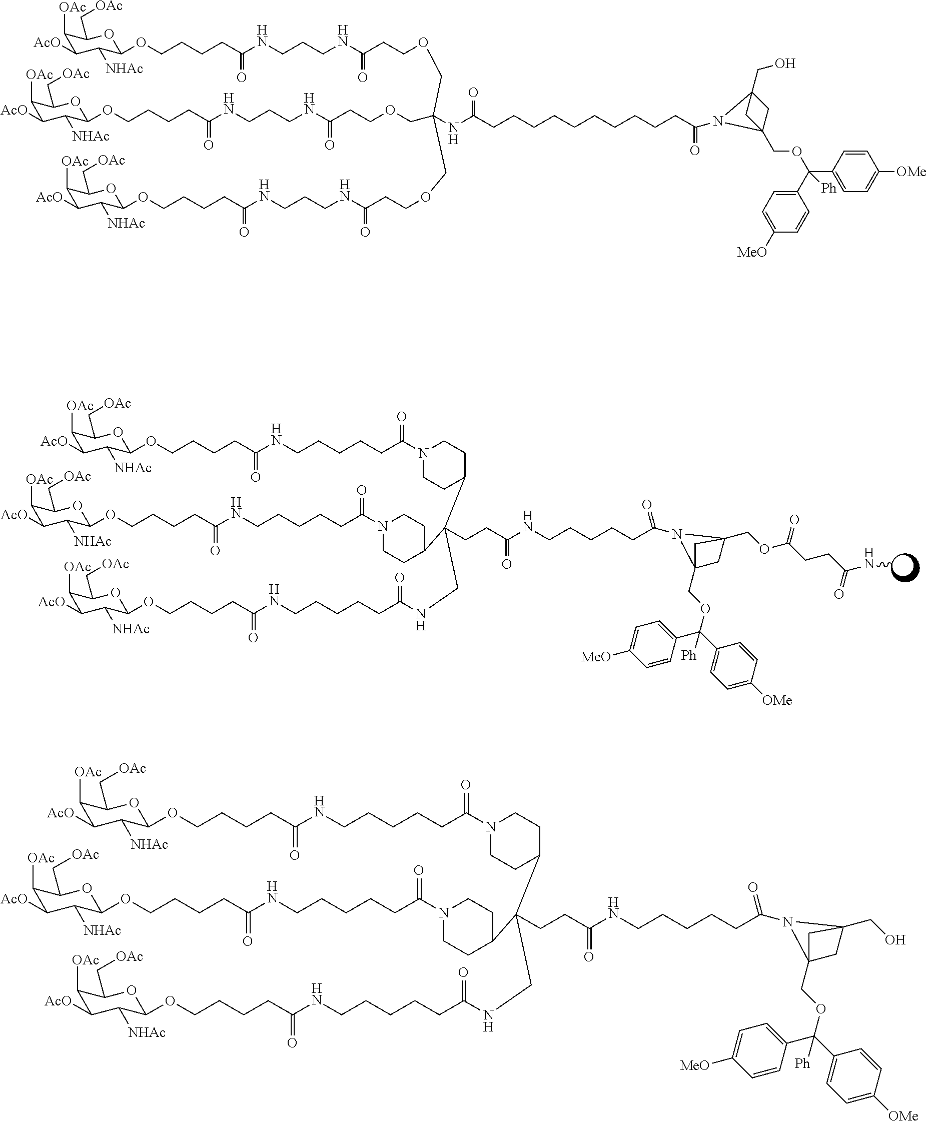 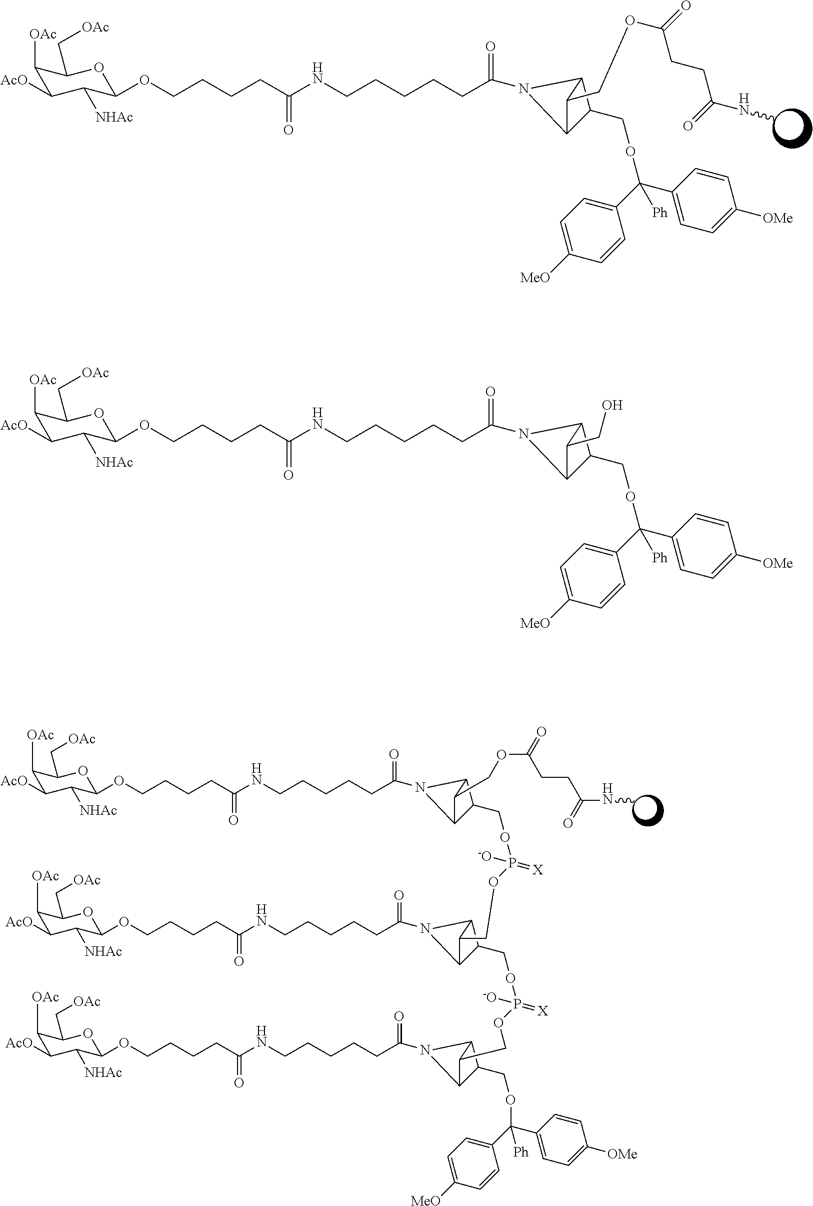 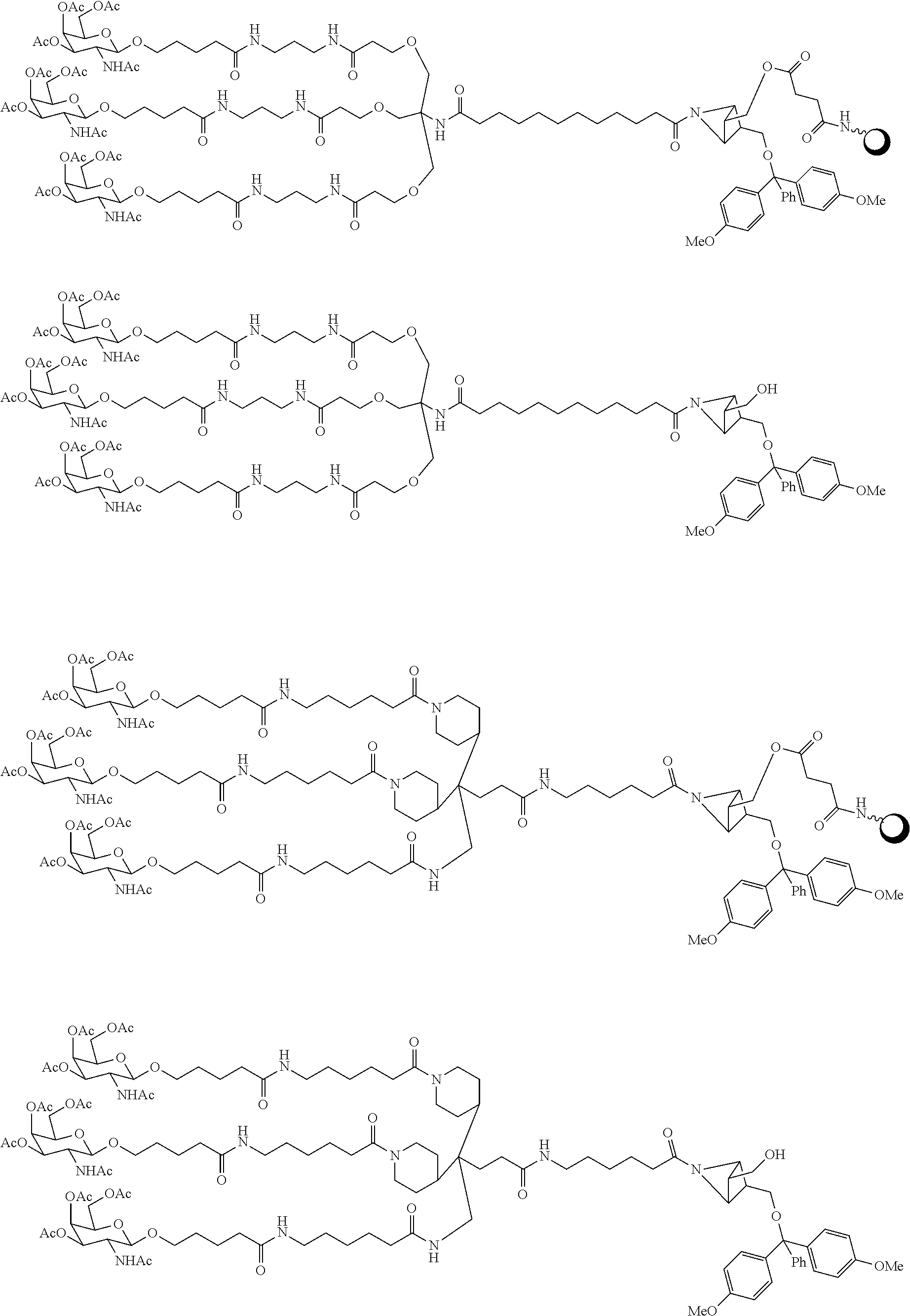 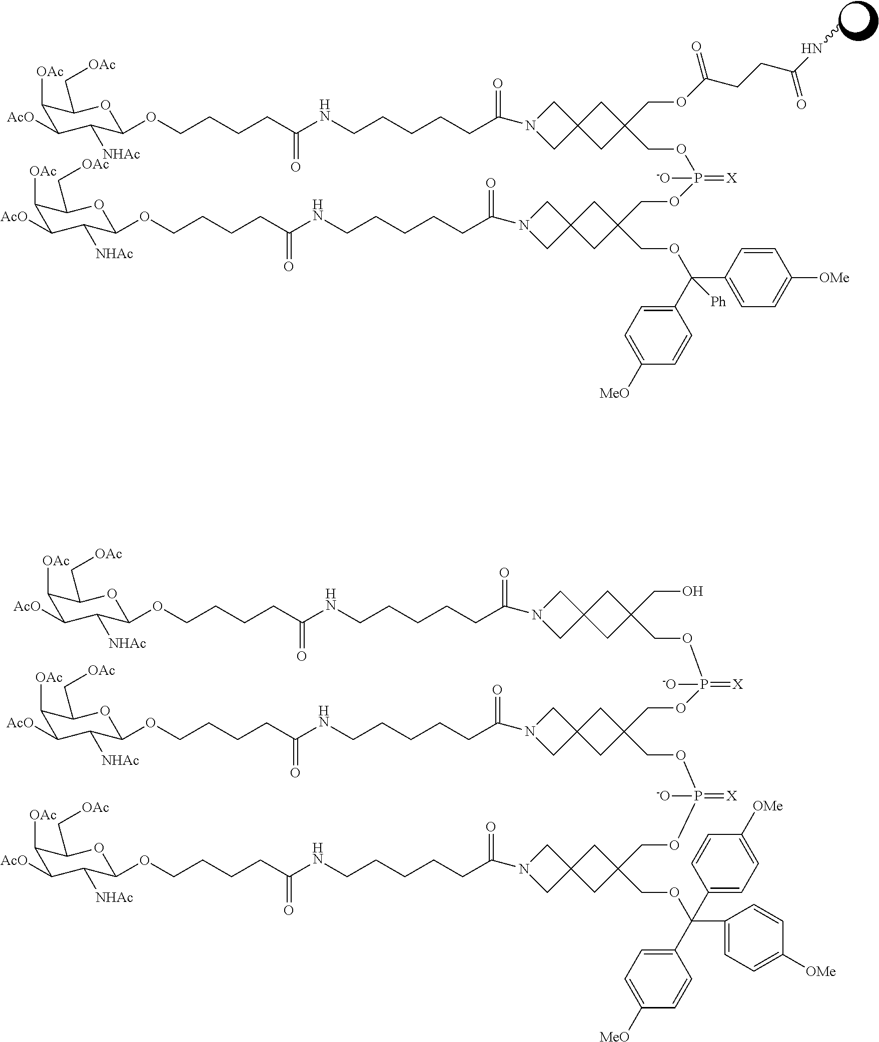 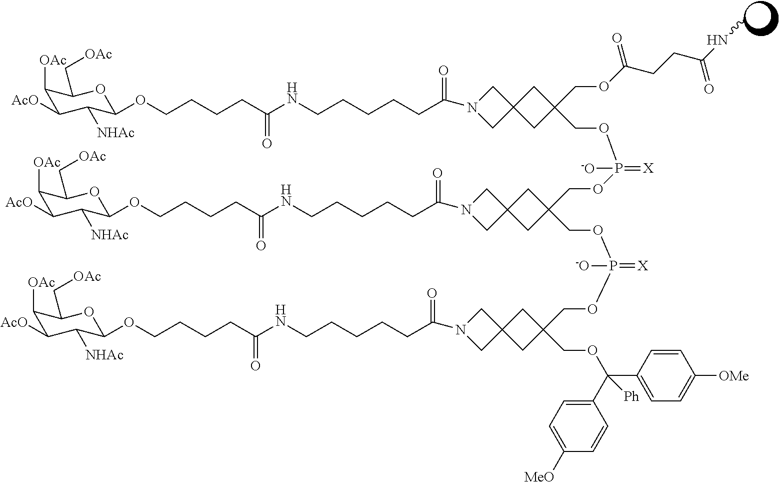 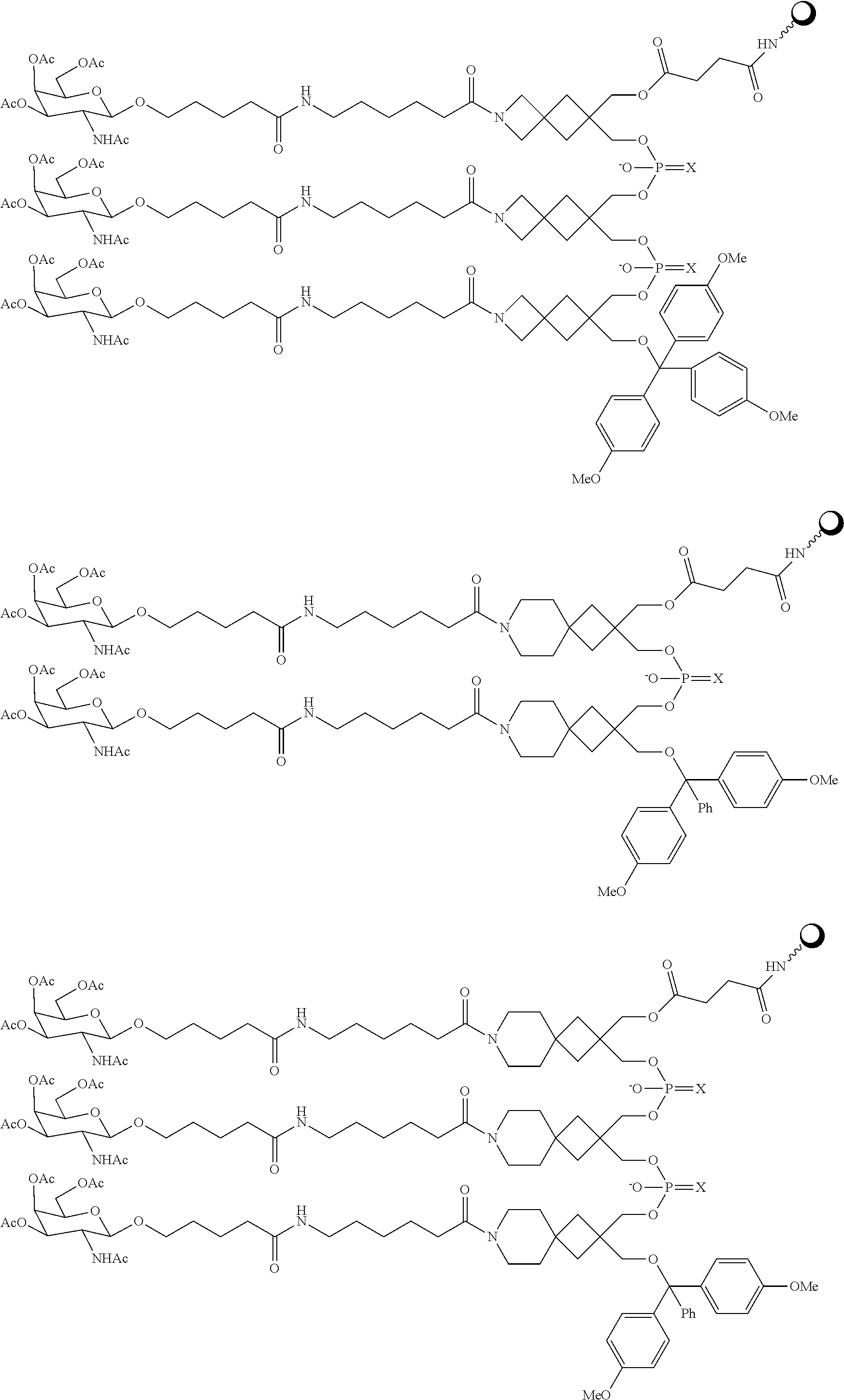 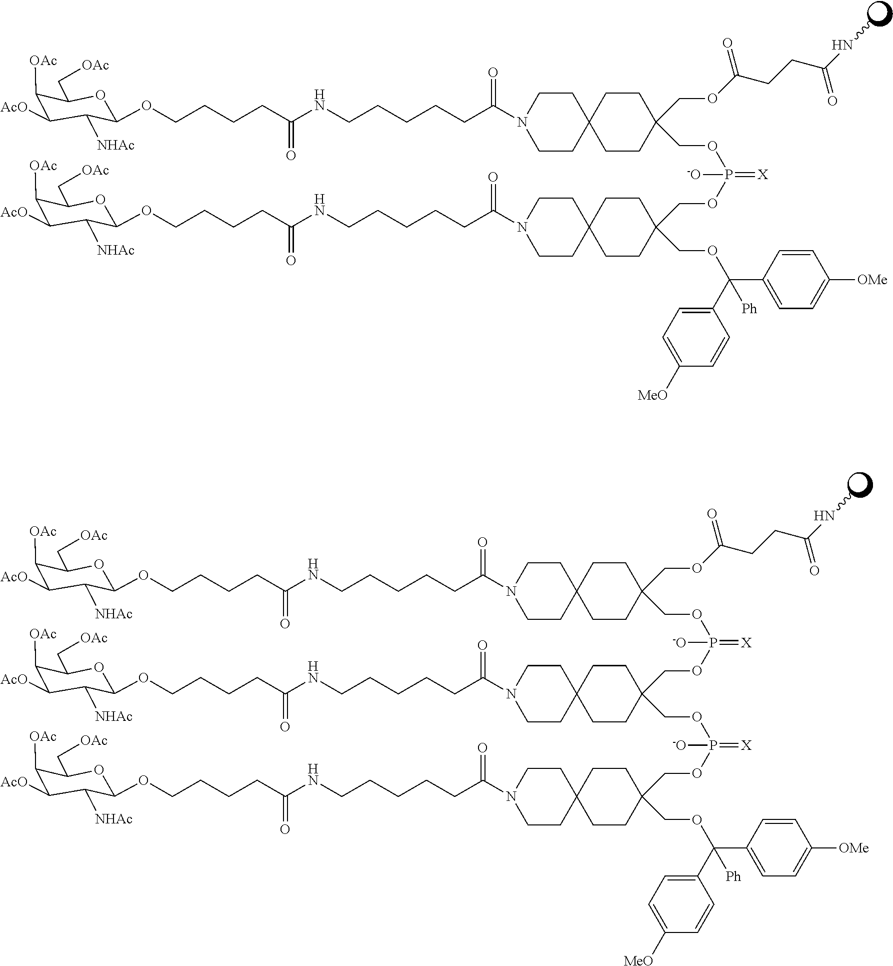 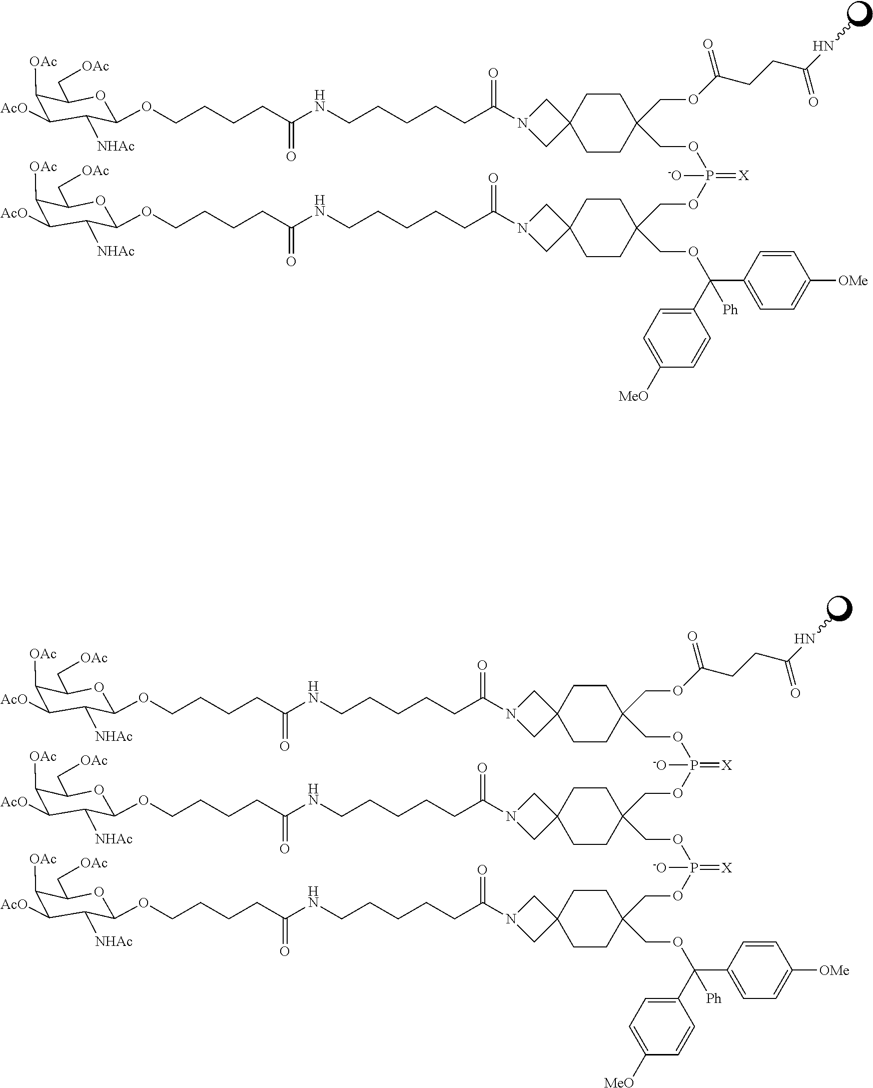 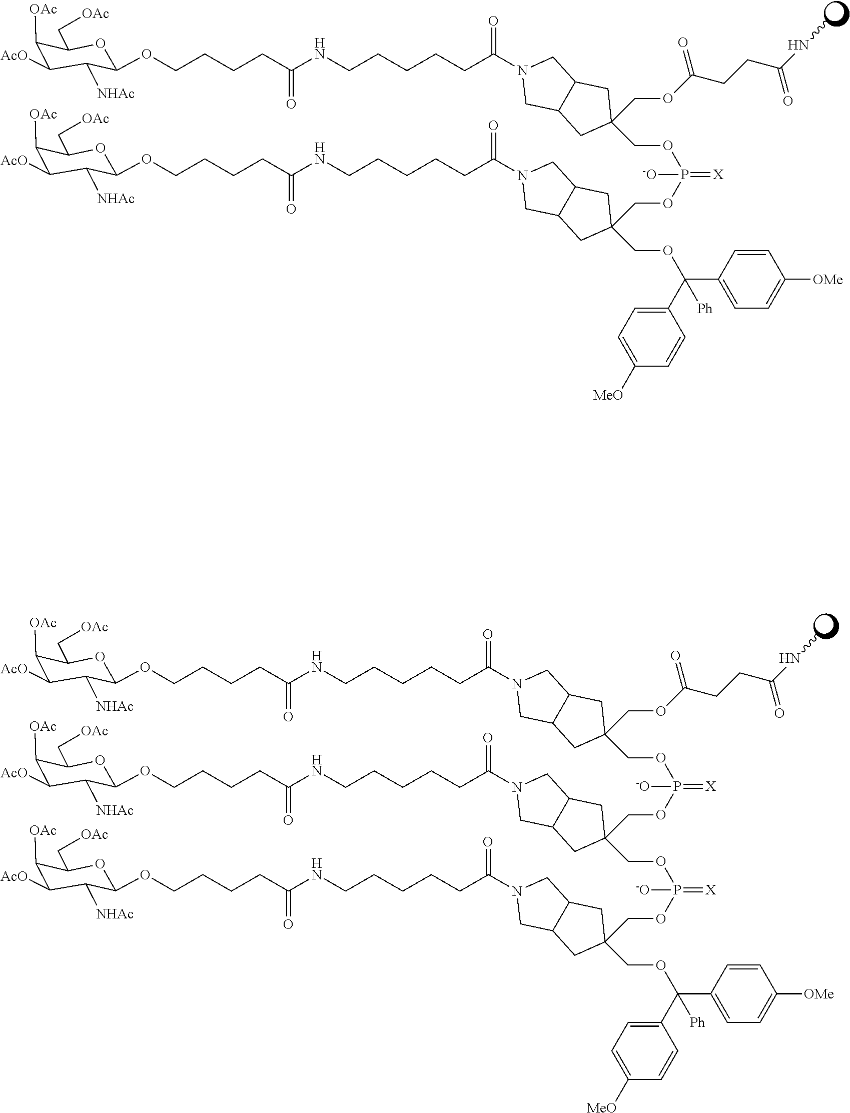 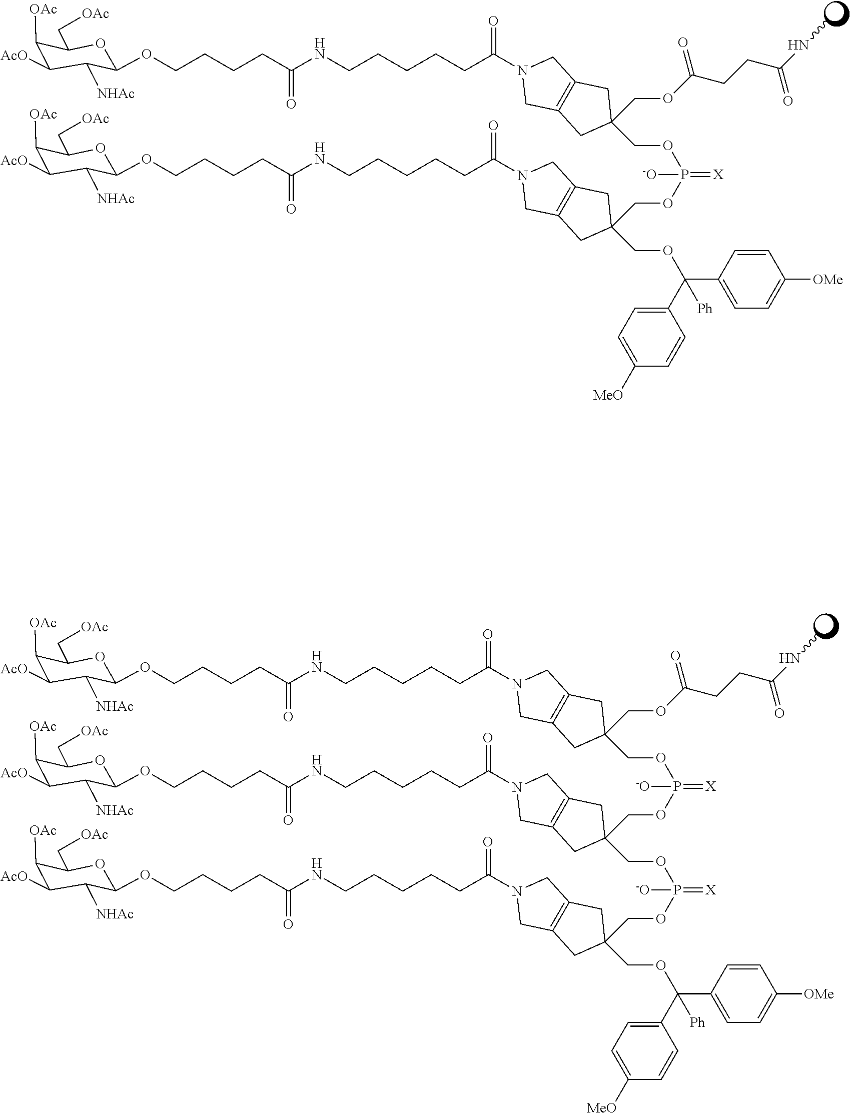 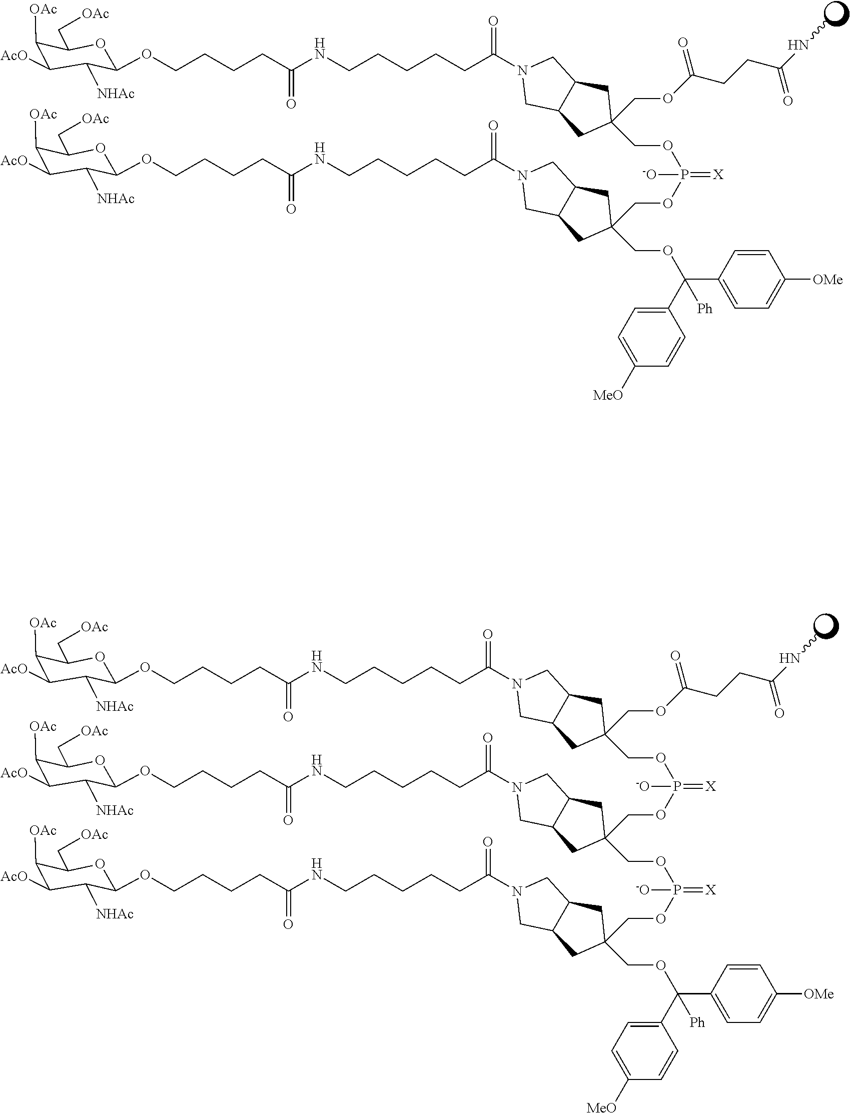 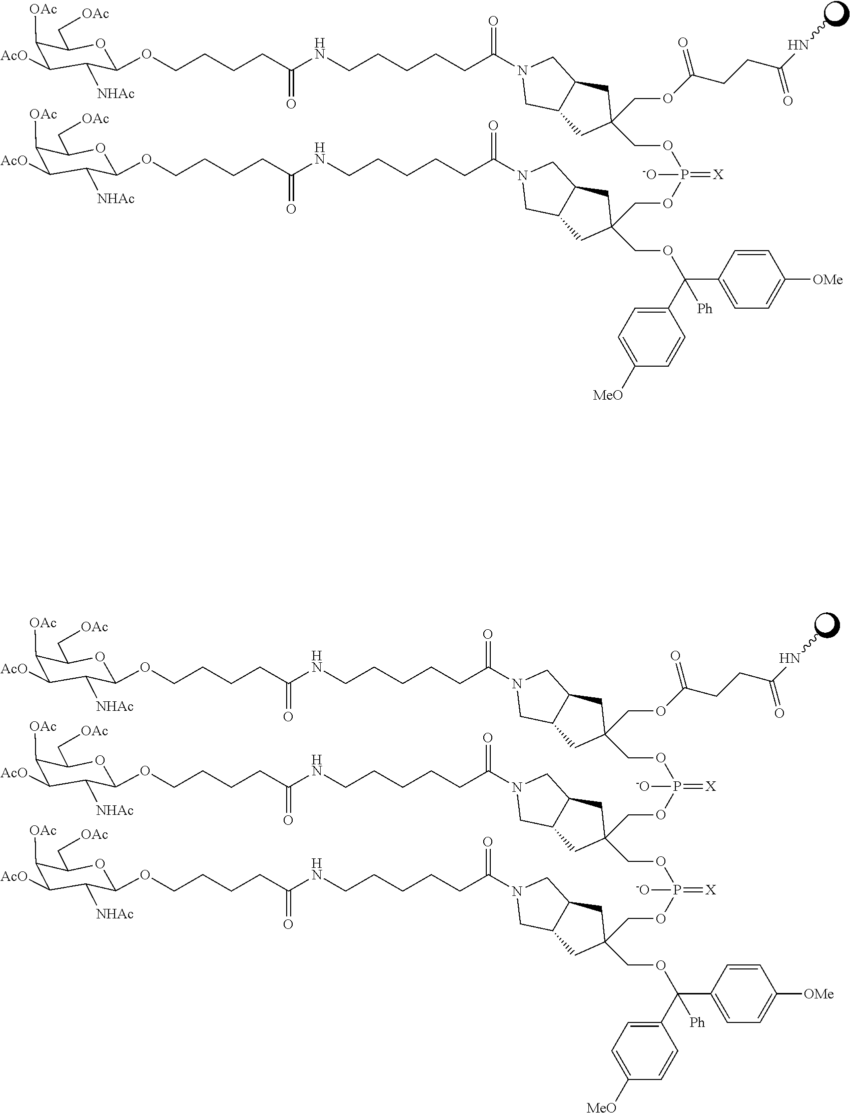 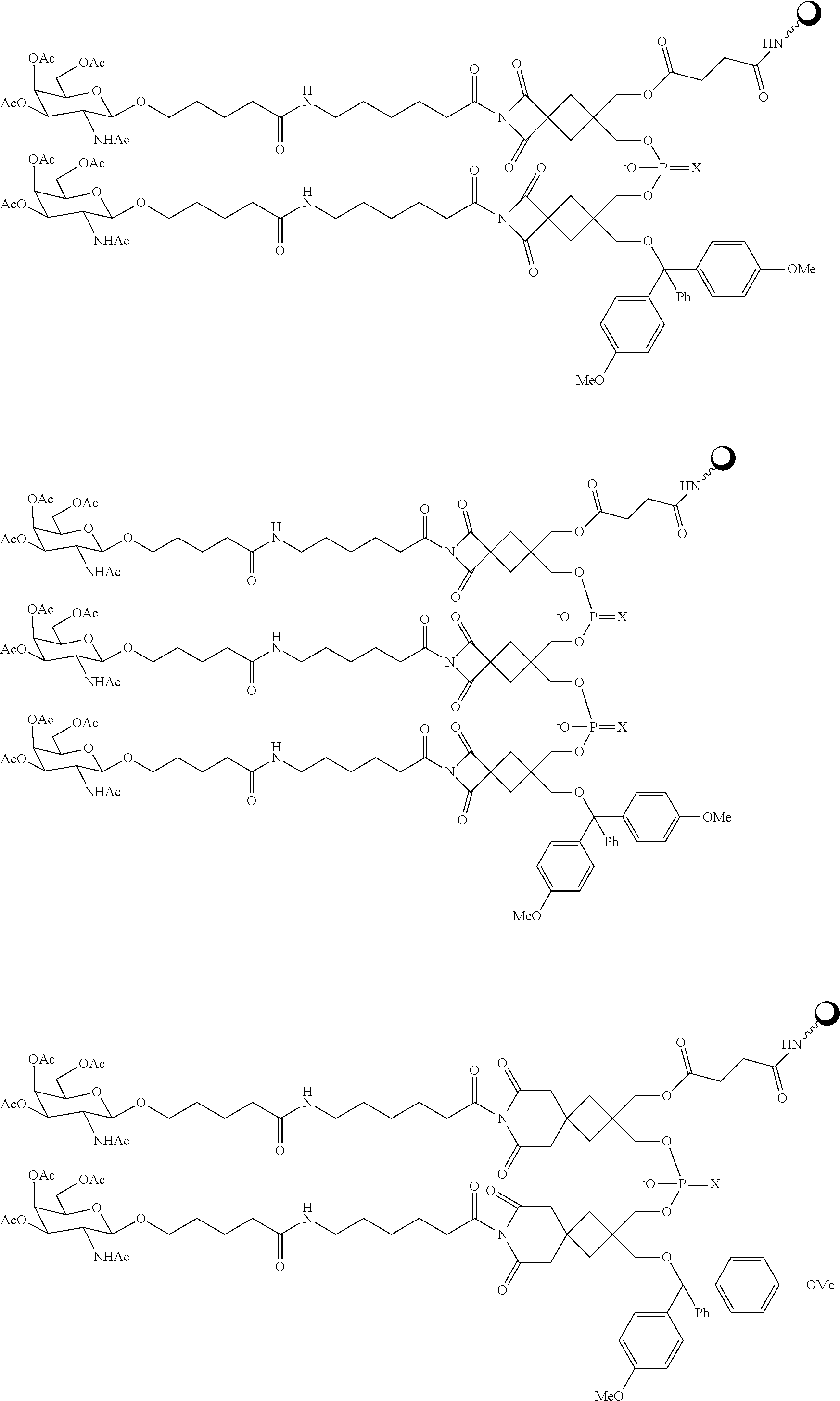 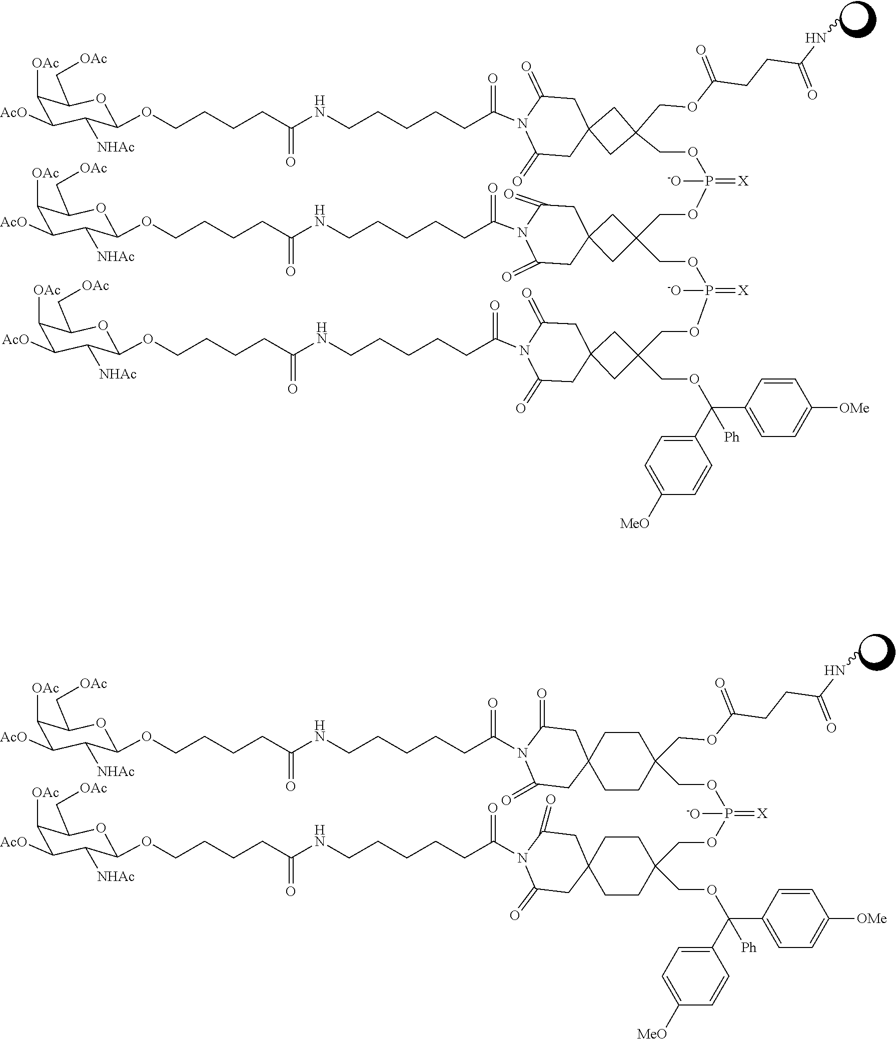 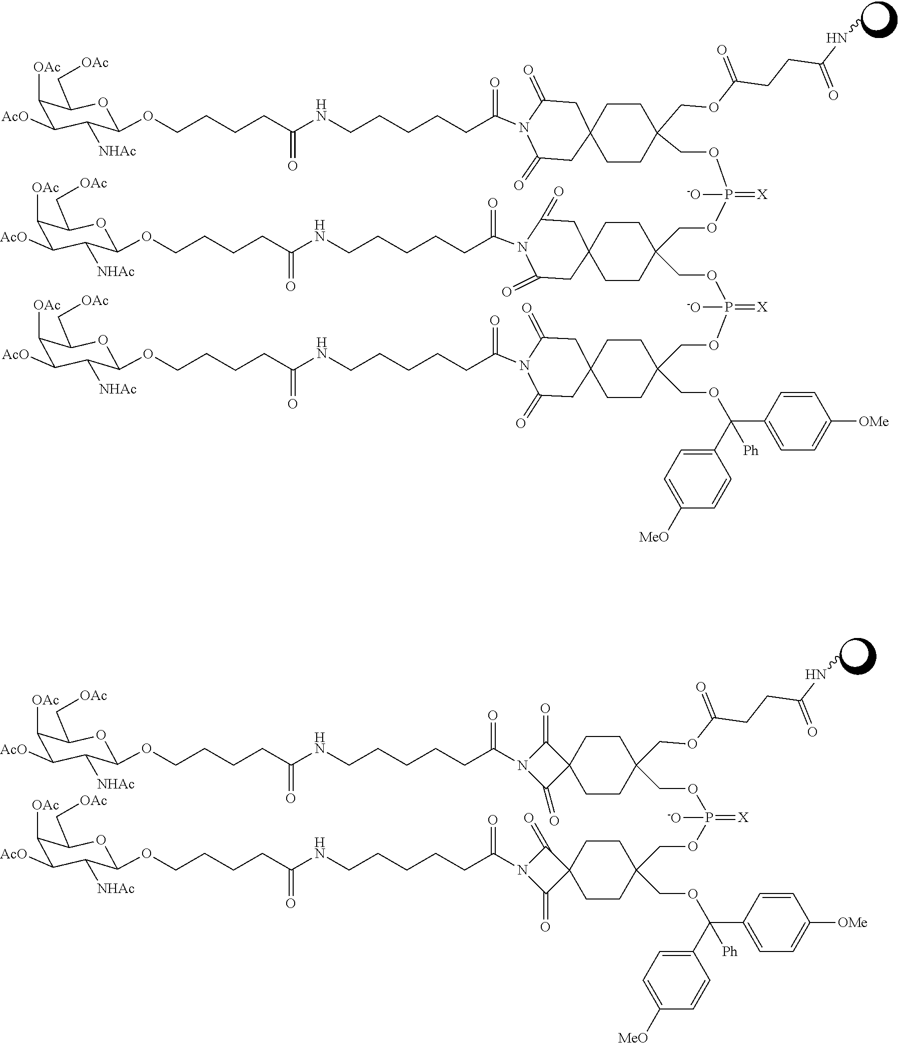 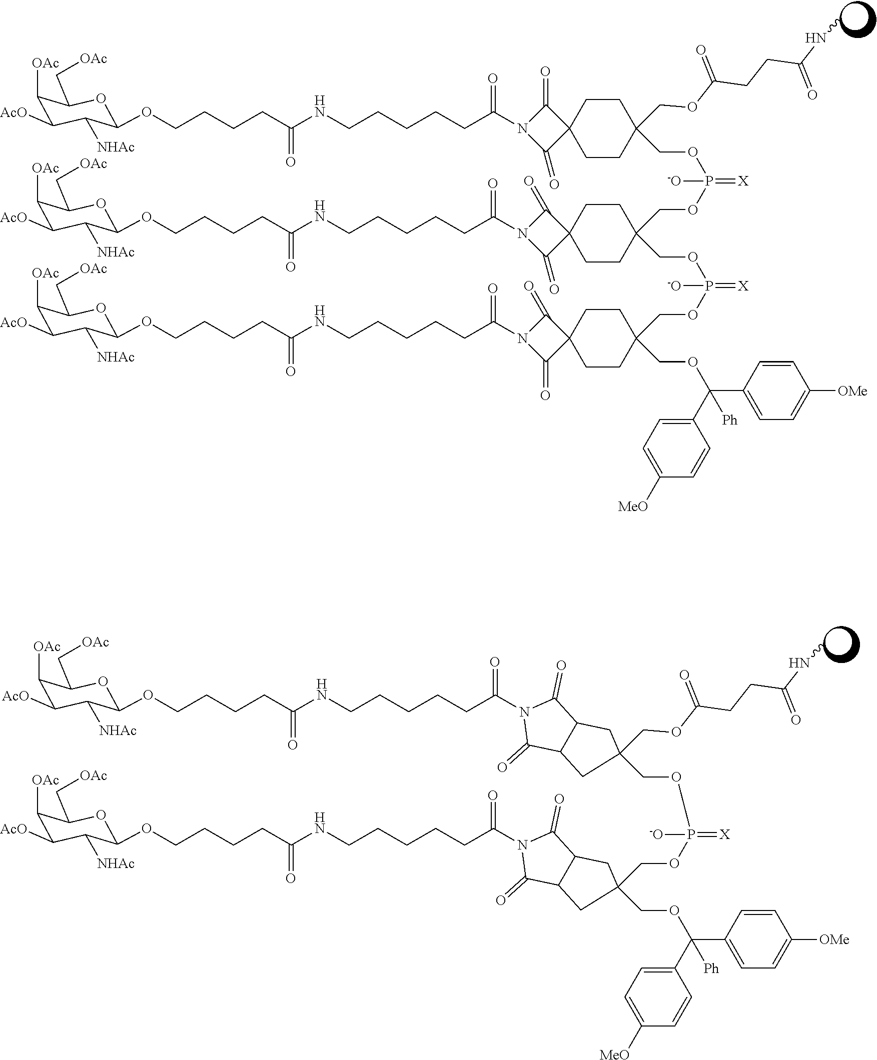 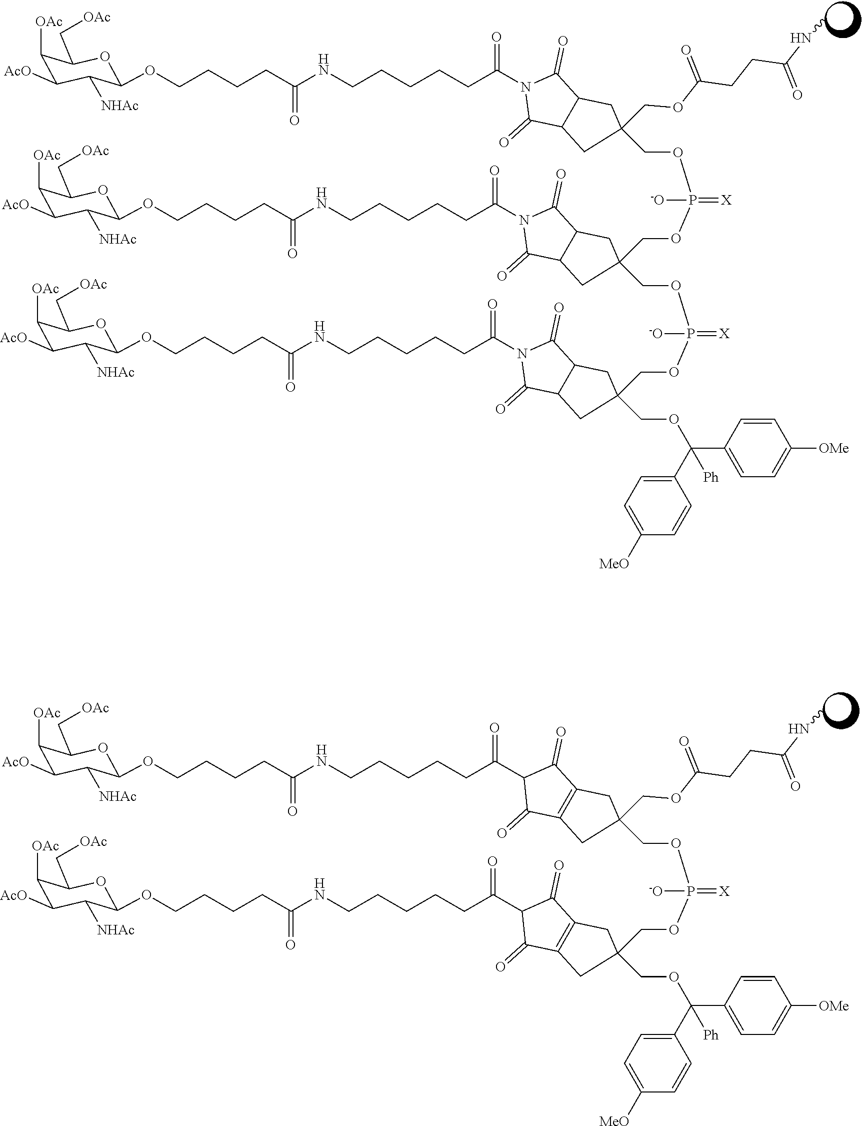 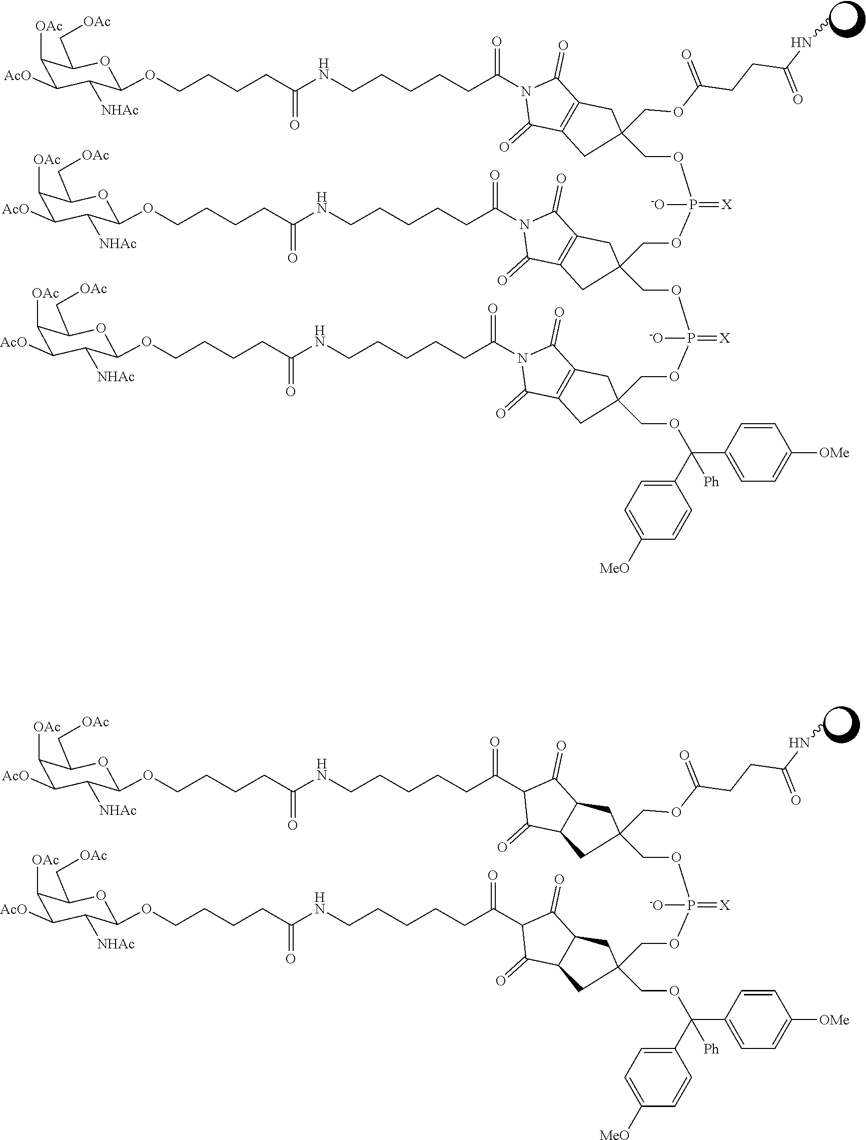 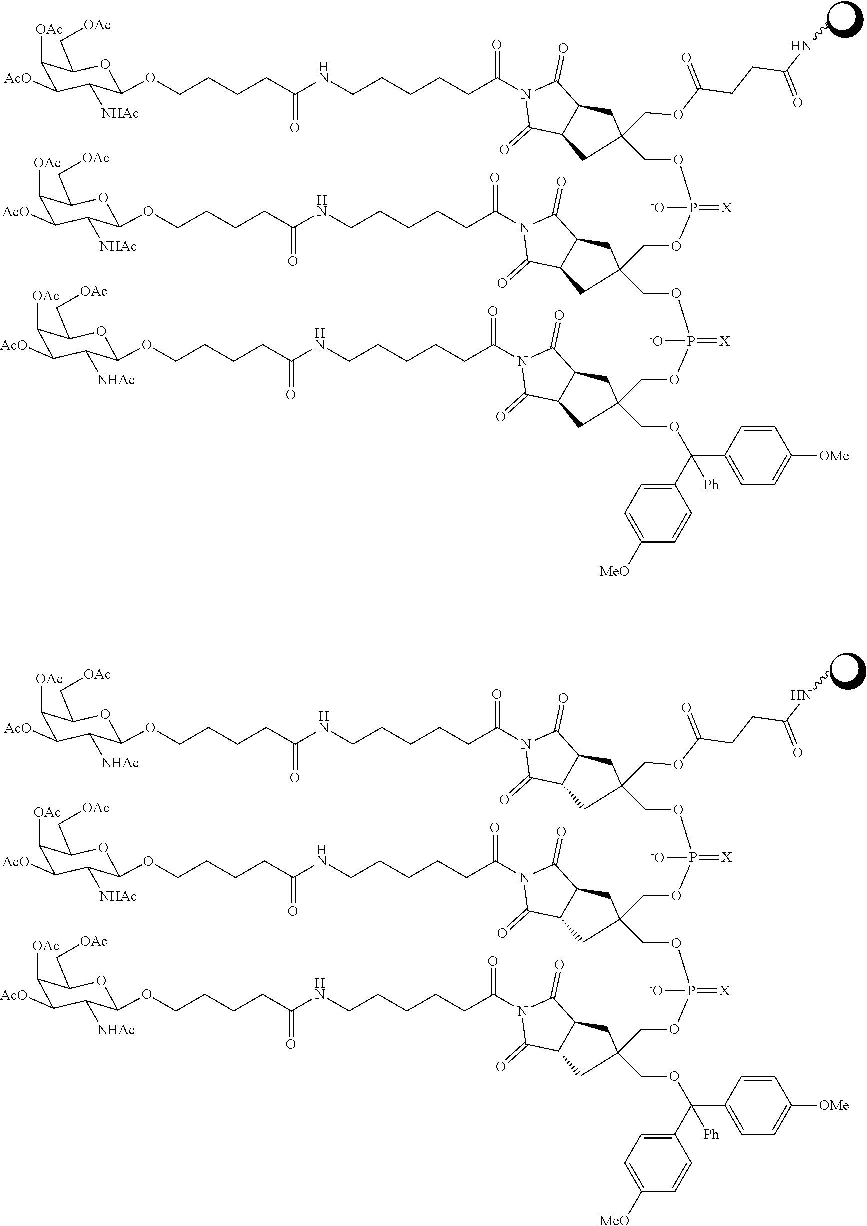 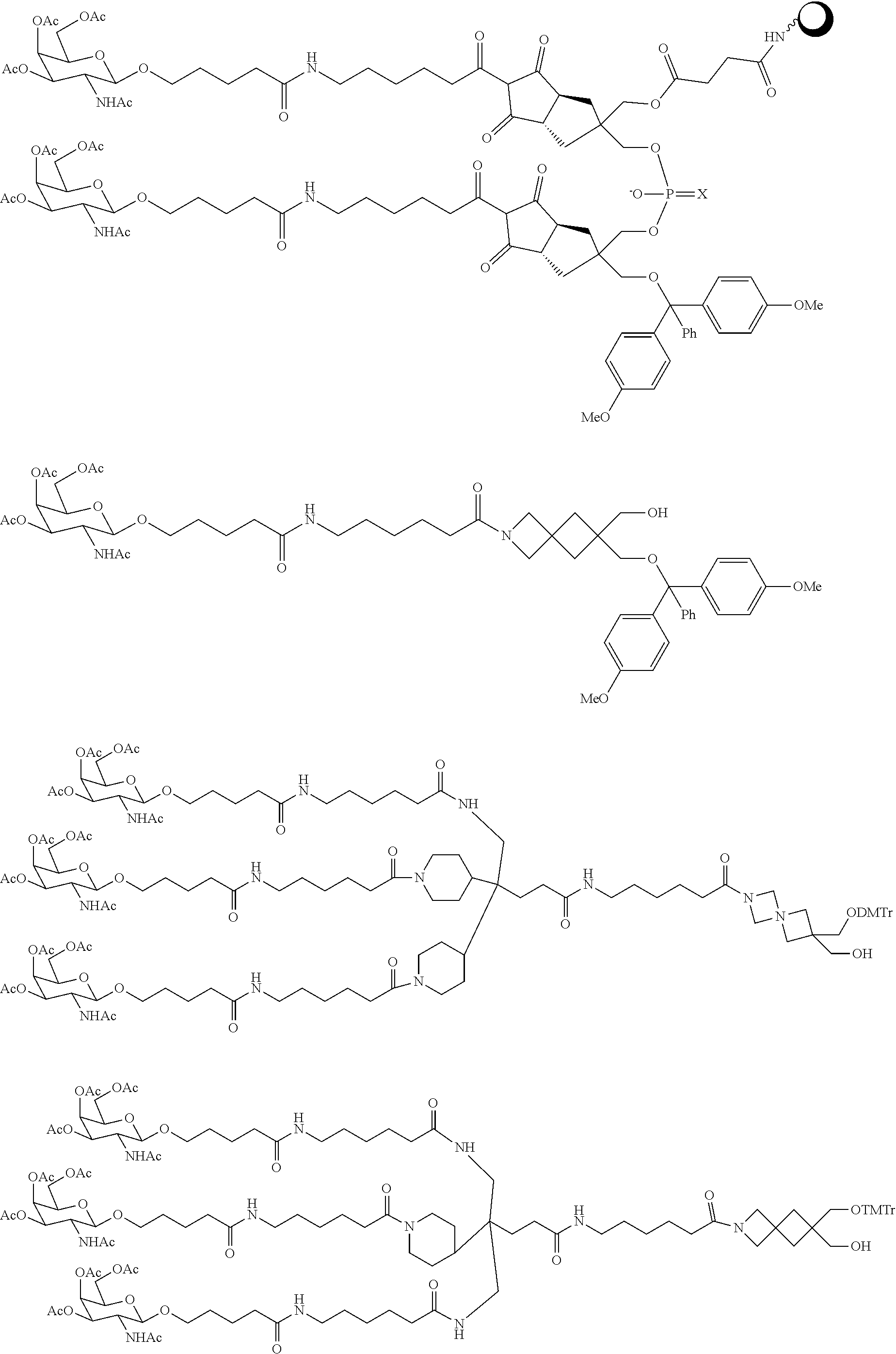 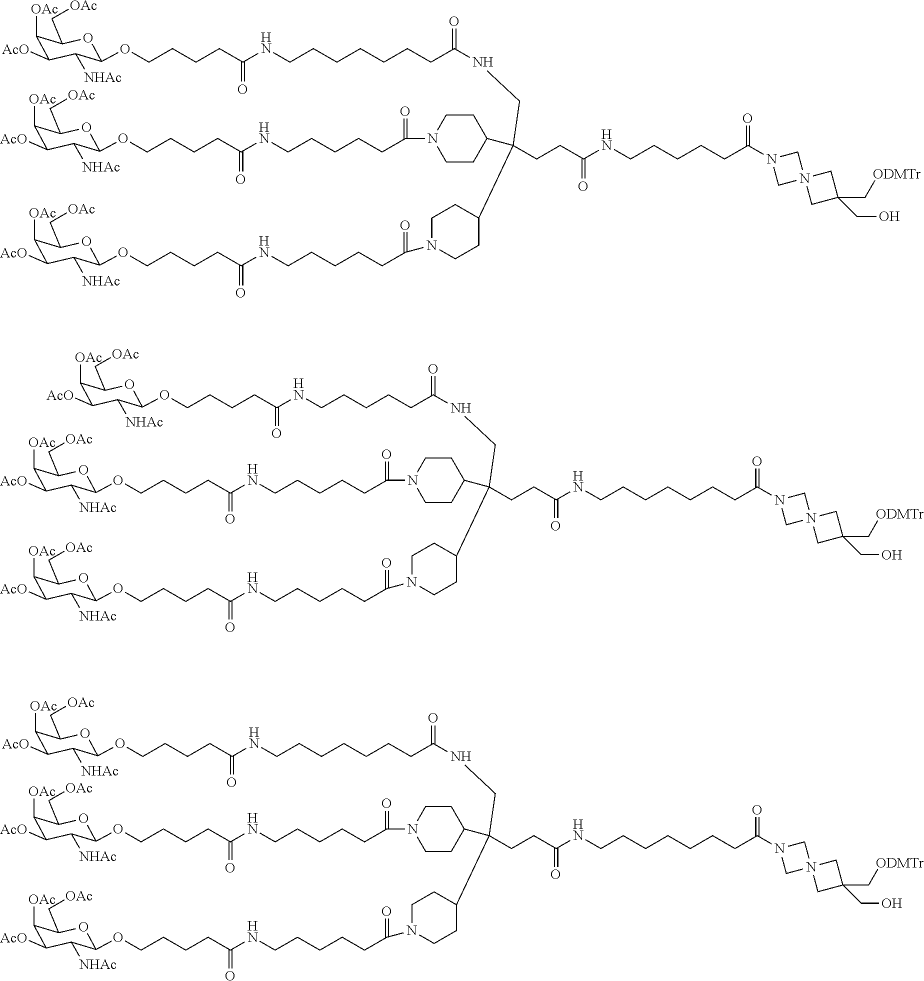 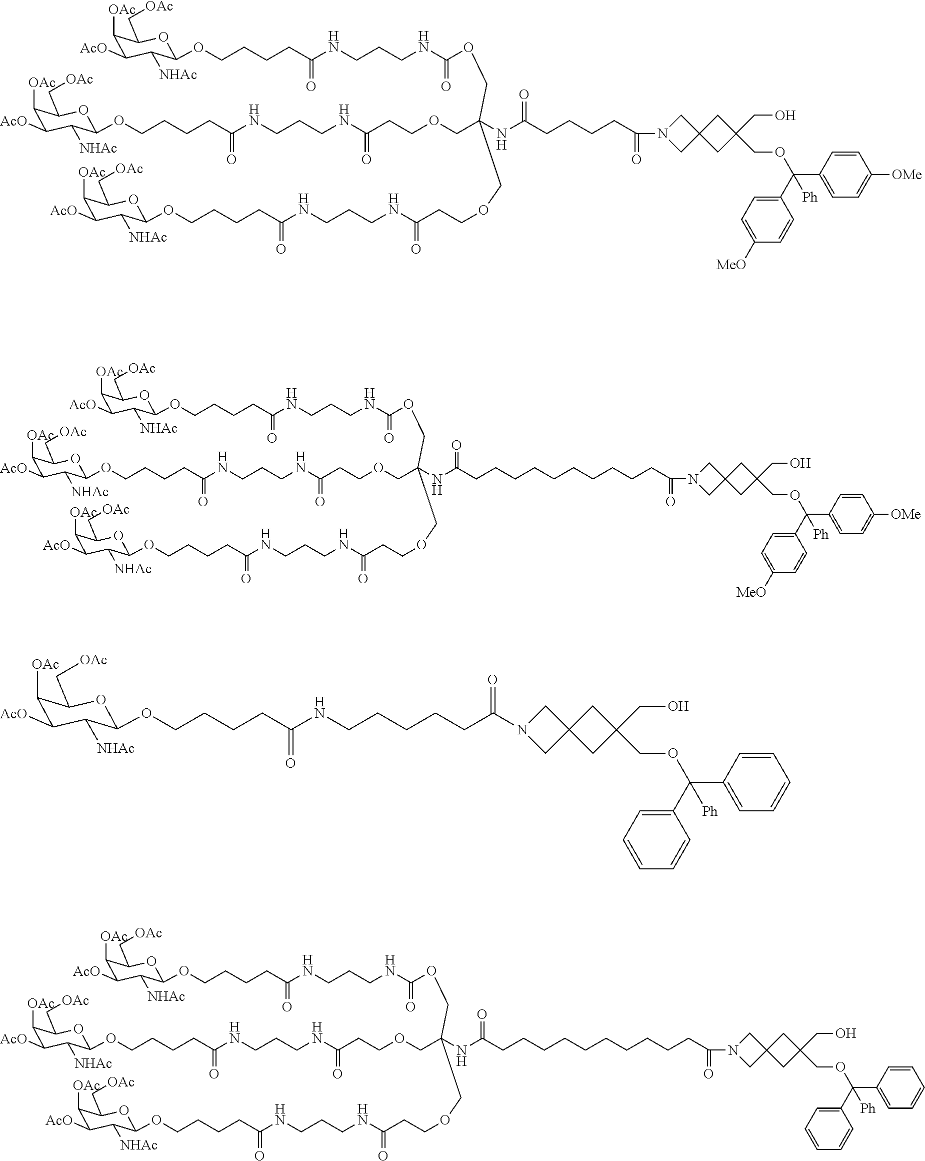 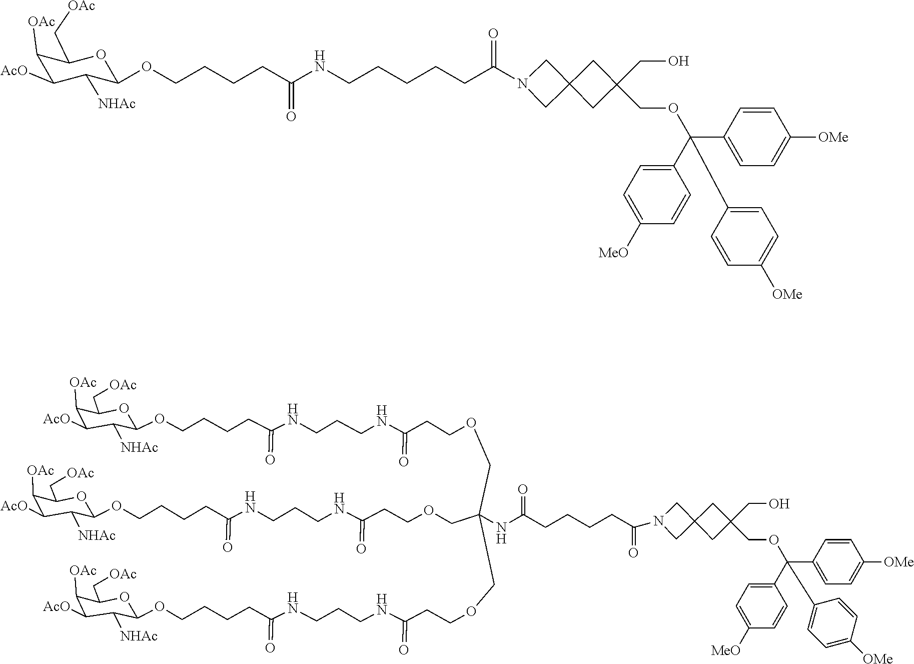 [SMILES: COc1ccc(C(OCC2(CO)CC3(CN(C(=O)CCCCCNC(=O)CCCCOC4OC(COC(C)=O)C(OC(C)=O)C(OC(C)=O)C4NC(C)=O)C3)C2)(c2ccc(OC)cc2)c2ccc(OC)cc2)cc1 ; COc1ccc(C(OCC2(CO)CC3(CN(C(=O)CCCCC(=O)NC(COCCC(=O)NCCCNC(=O)CCCCOC4OC(COC(C)=O)C(OC(C)=O)C(OC(C)=O)C4NC(C)=O)(COCCC(=O)NCCCNC(=O)CCCCOC4OC(COC(C)=O)C(OC(C)=O)C(OC(C)=O)C4NC(C)=O)COCCC(=O)NCCCNC(=O)CCCCOC4OC(COC(C)=O)C(OC(C)=O)C(OC(C)=O)C4NC(C)=O)C3)C2)(c2ccc(OC)cc2)c2ccc(OC)cc2)cc1] 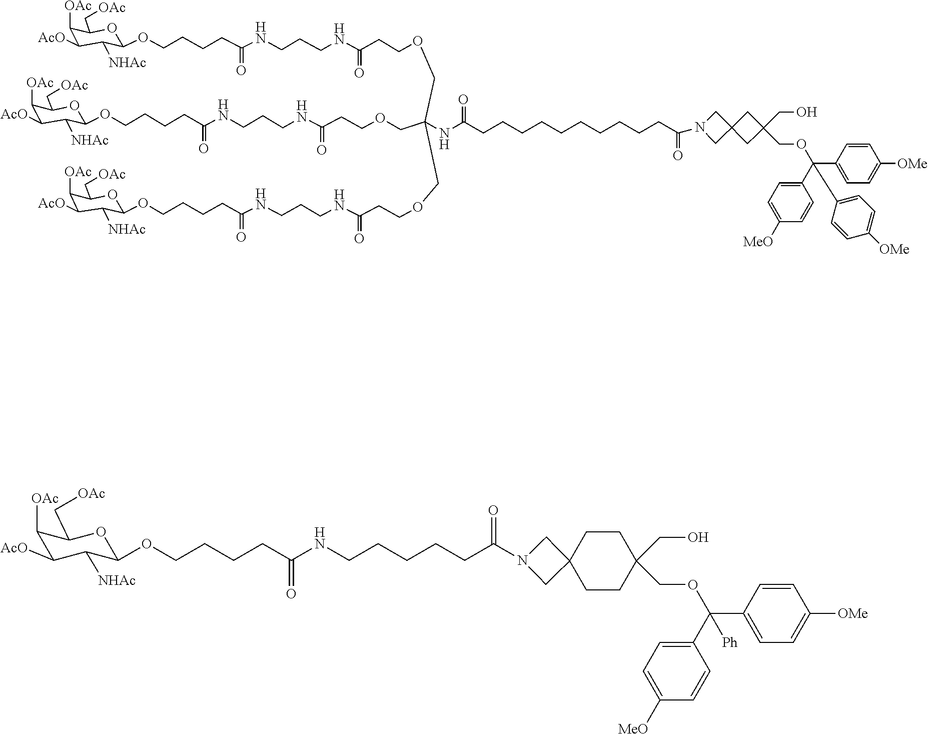 [SMILES: COc1ccc(C(OCC2(CO)CC3(CN(C(=O)CCCCCCCCCCC(=O)NC(COCCC(=O)NCCCNC(=O)CCCCOC4OC(COC(C)=O)C(OC(C)=O)C(OC(C)=O)C4NC(C)=O)(COCCC(=O)NCCCNC(=O)CCCCOC4OC(COC(C)=O)C(OC(C)=O)C(OC(C)=O)C4NC(C)=O)COCCC(=O)NCCCNC(=O)CCCCOC4OC(COC(C)=O)C(OC(C)=O)C(OC(C)=O)C4NC(C)=O)C3)C2)(c2ccc(OC)cc2)c2ccc(OC)cc2)cc1 ; COc1ccc(C(OCC2(CO)CCC3(CC2)CN(C(=O)CCCCCNC(=O)CCCCOC2OC(COC(C)=O)C(OC(C)=O)C(OC(C)=O)C2NC(C)=O)C3)(c2ccccc2)c2ccc(OC)cc2)cc1] 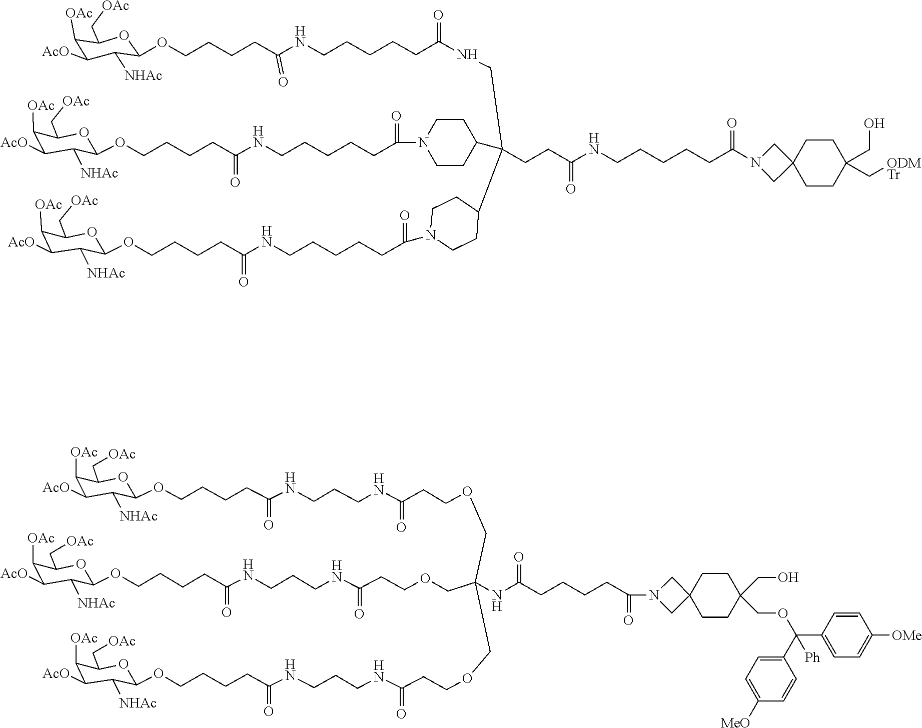 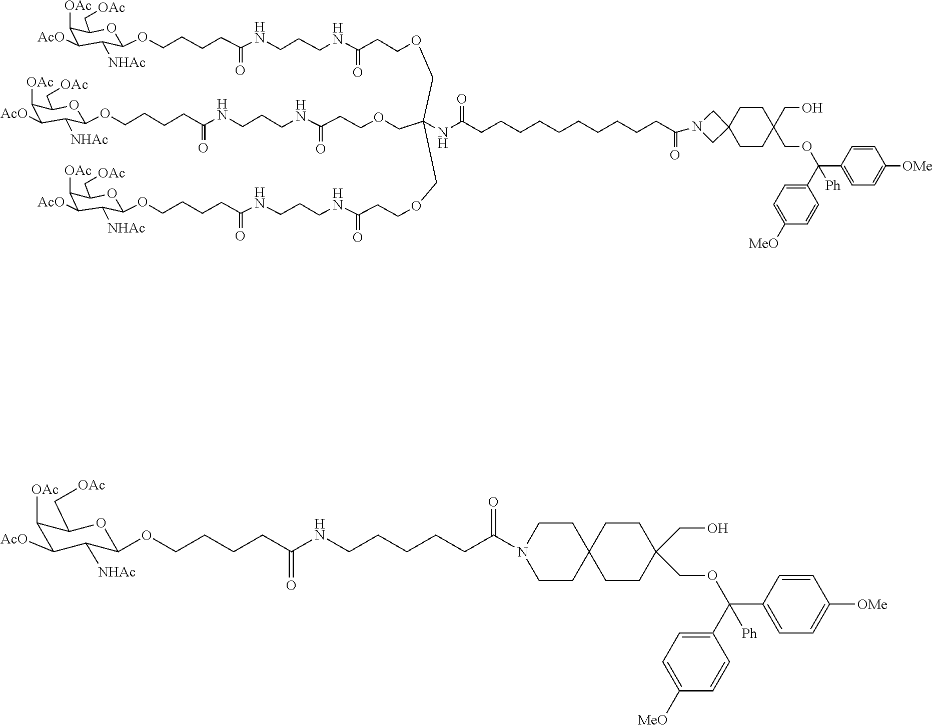 [SMILES: COc1ccc(C(OCC2(CO)CCC3(CC2)CN(C(=O)CCCCCCCCCCC(=O)NC(COCCC(=O)NCCCNC(=O)CCCCOC2OC(COC(C)=O)C(OC(C)=O)C(OC(C)=O)C2NC(C)=O)(COCCC(=O)NCCCNC(=O)CCCCOC2OC(COC(C)=O)C(OC(C)=O)C(OC(C)=O)C2NC(C)=O)COCCC(=O)NCCCNC(=O)CCCCOC2OC(COC(C)=O)C(OC(C)=O)C(OC(C)=O)C2NC(C)=O)C3)(c2ccccc2)c2ccc(OC)cc2)cc1 ; COc1ccc(C(OCC2(CO)CCC3(CCN(C(=O)CCCCCNC(=O)CCCCOC4OC(COC(C)=O)C(OC(C)=O)C(OC(C)=O)C4NC(C)=O)CC3)CC2)(c2ccccc2)c2ccc(OC)cc2)cc1] 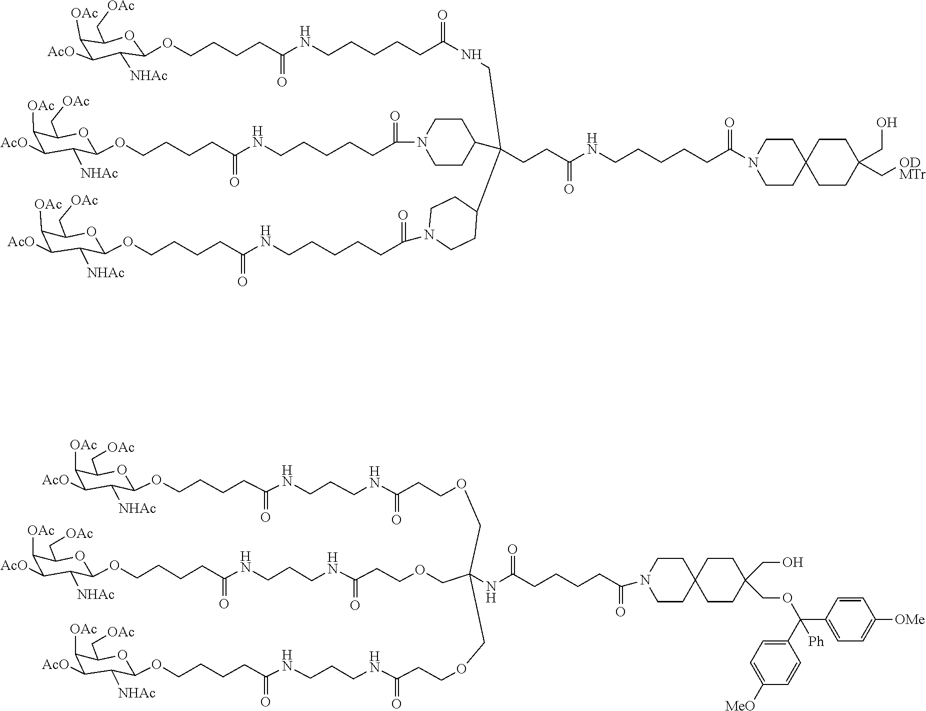 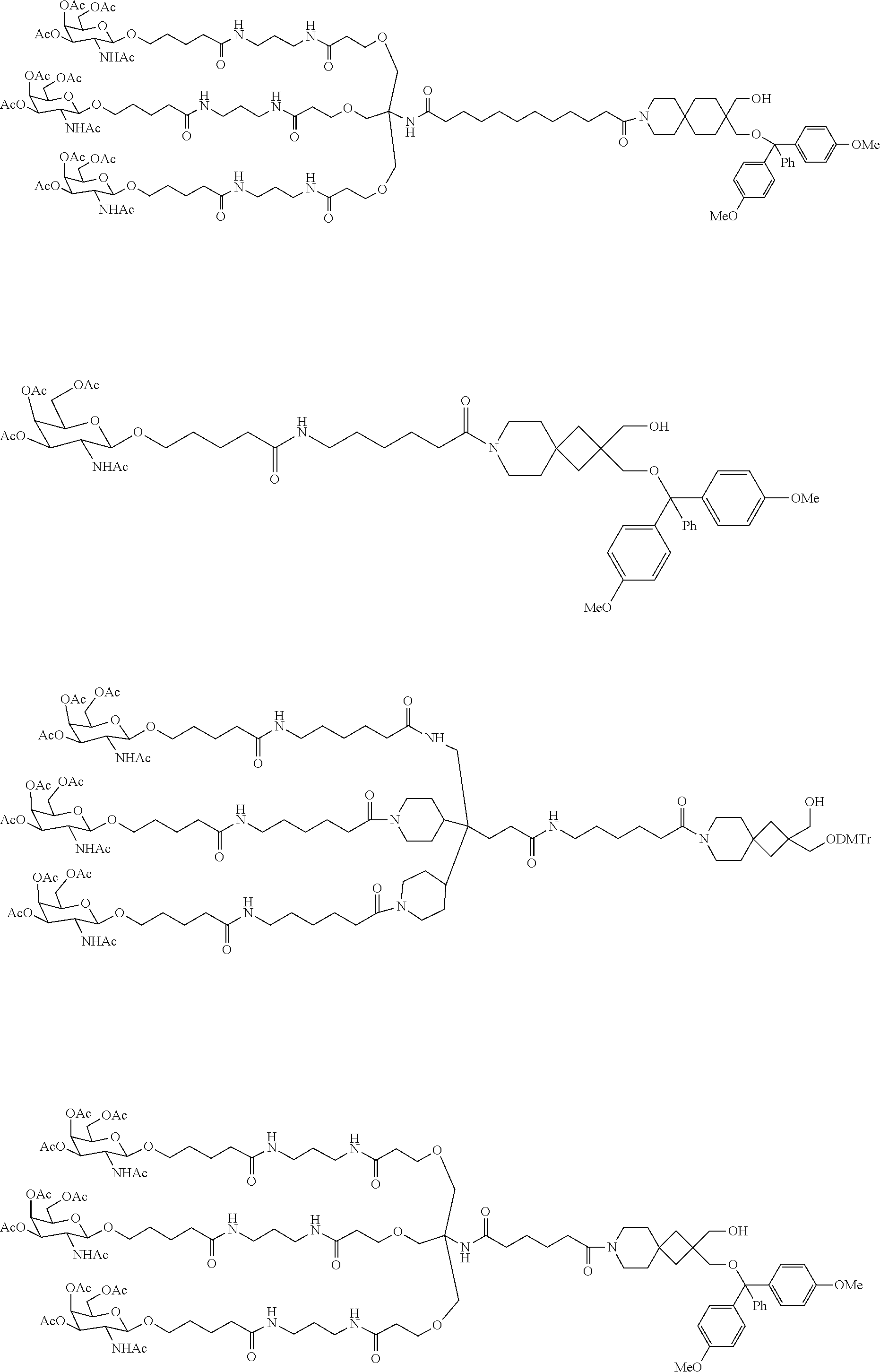 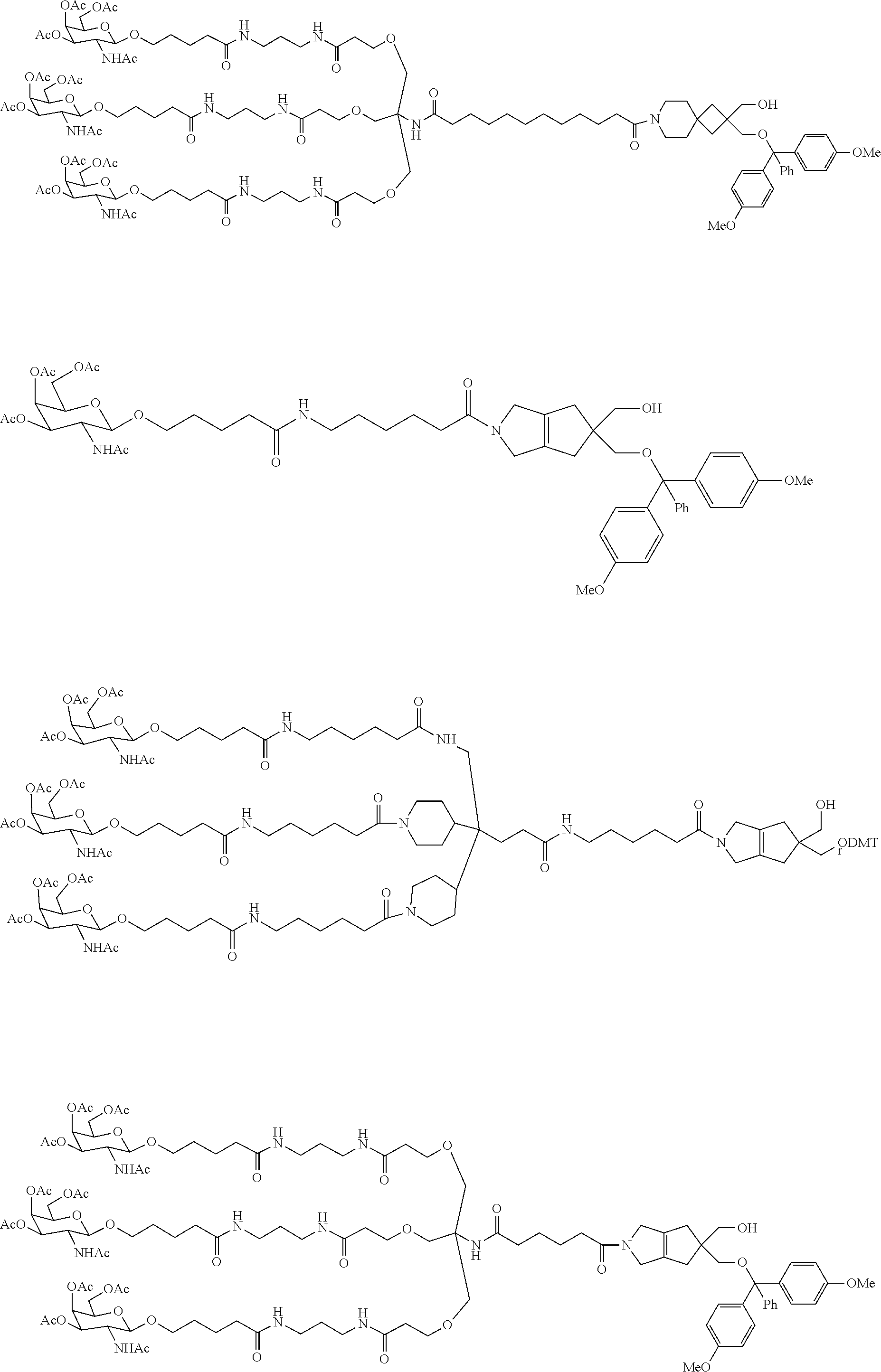 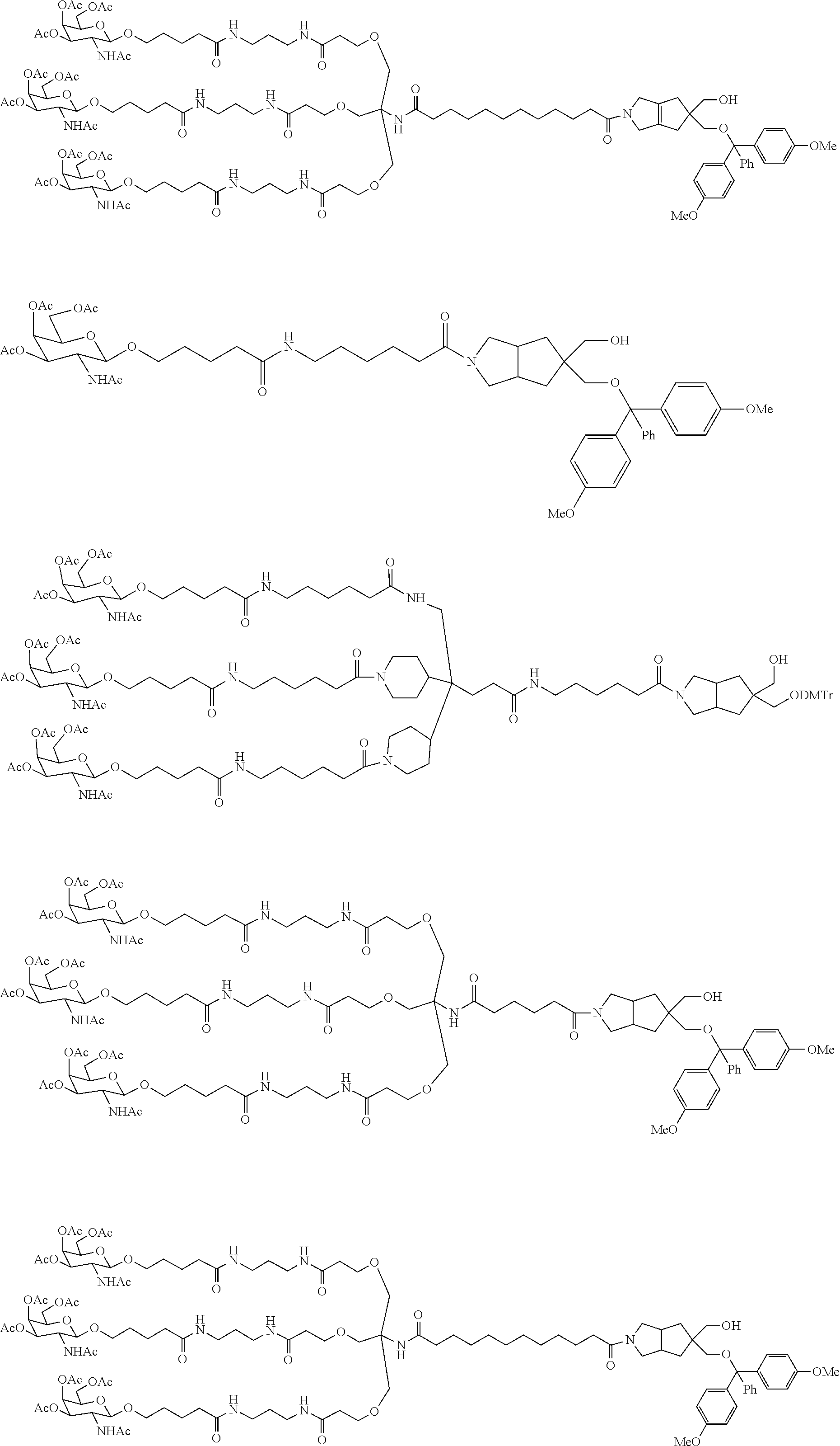 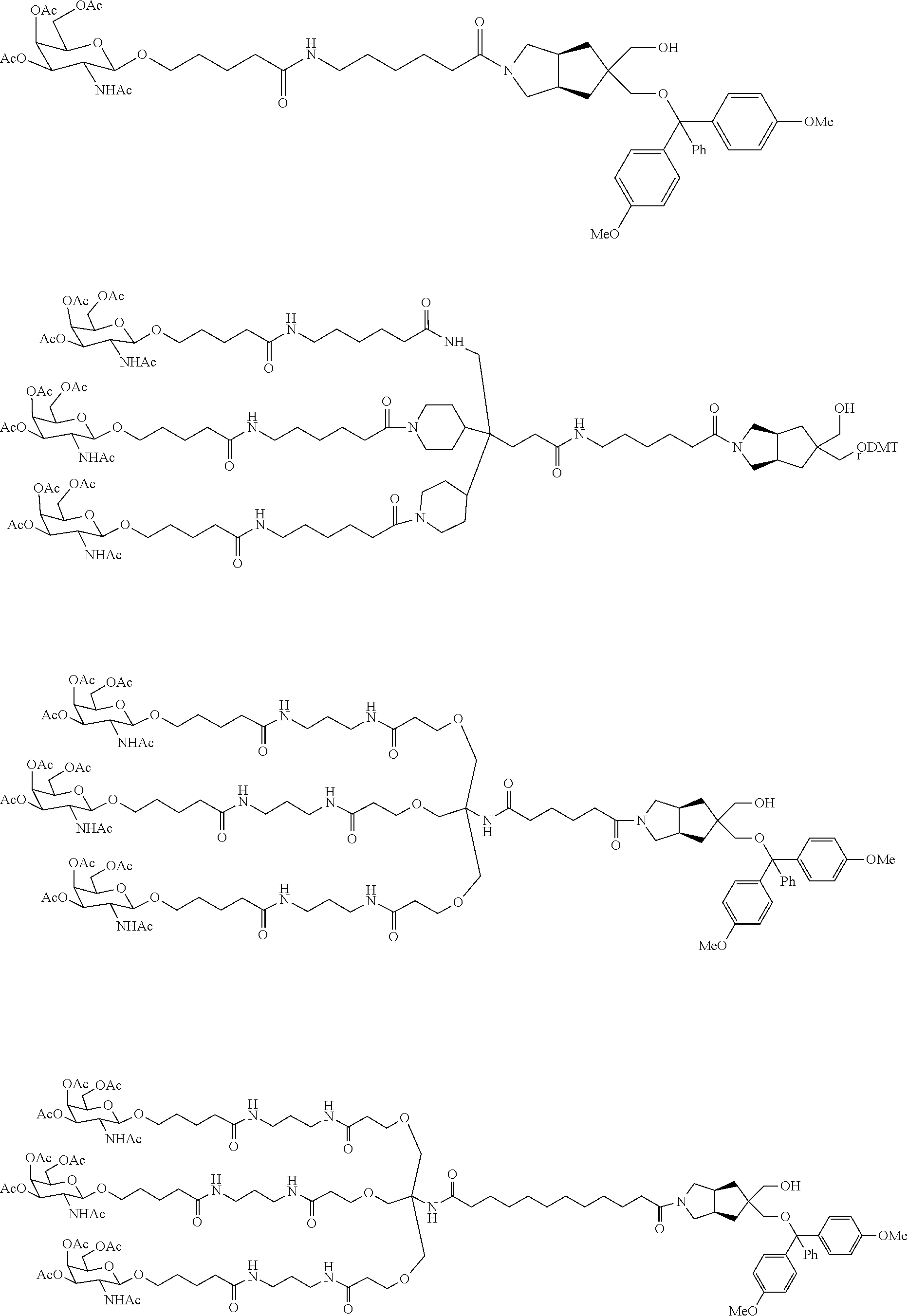 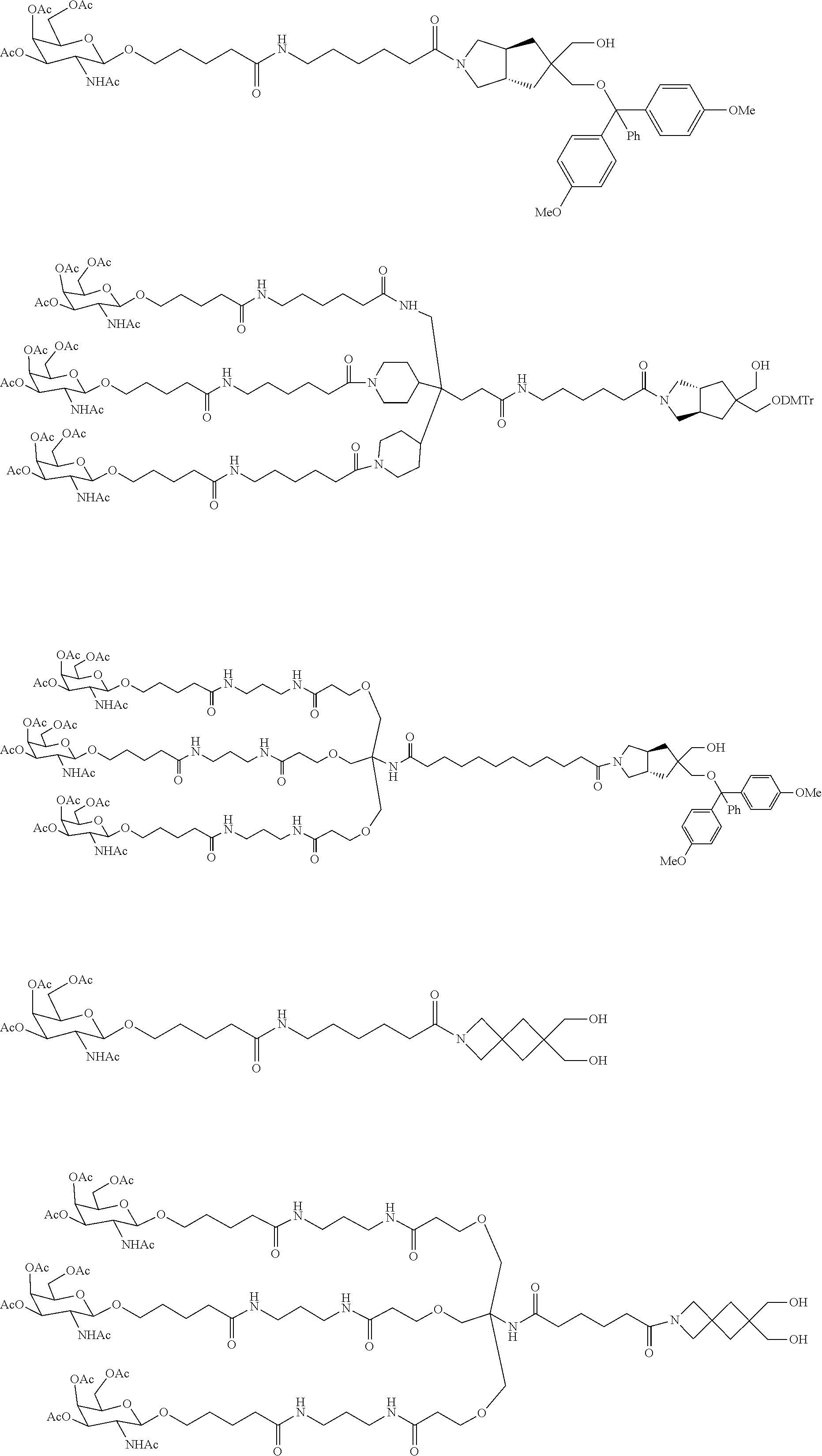 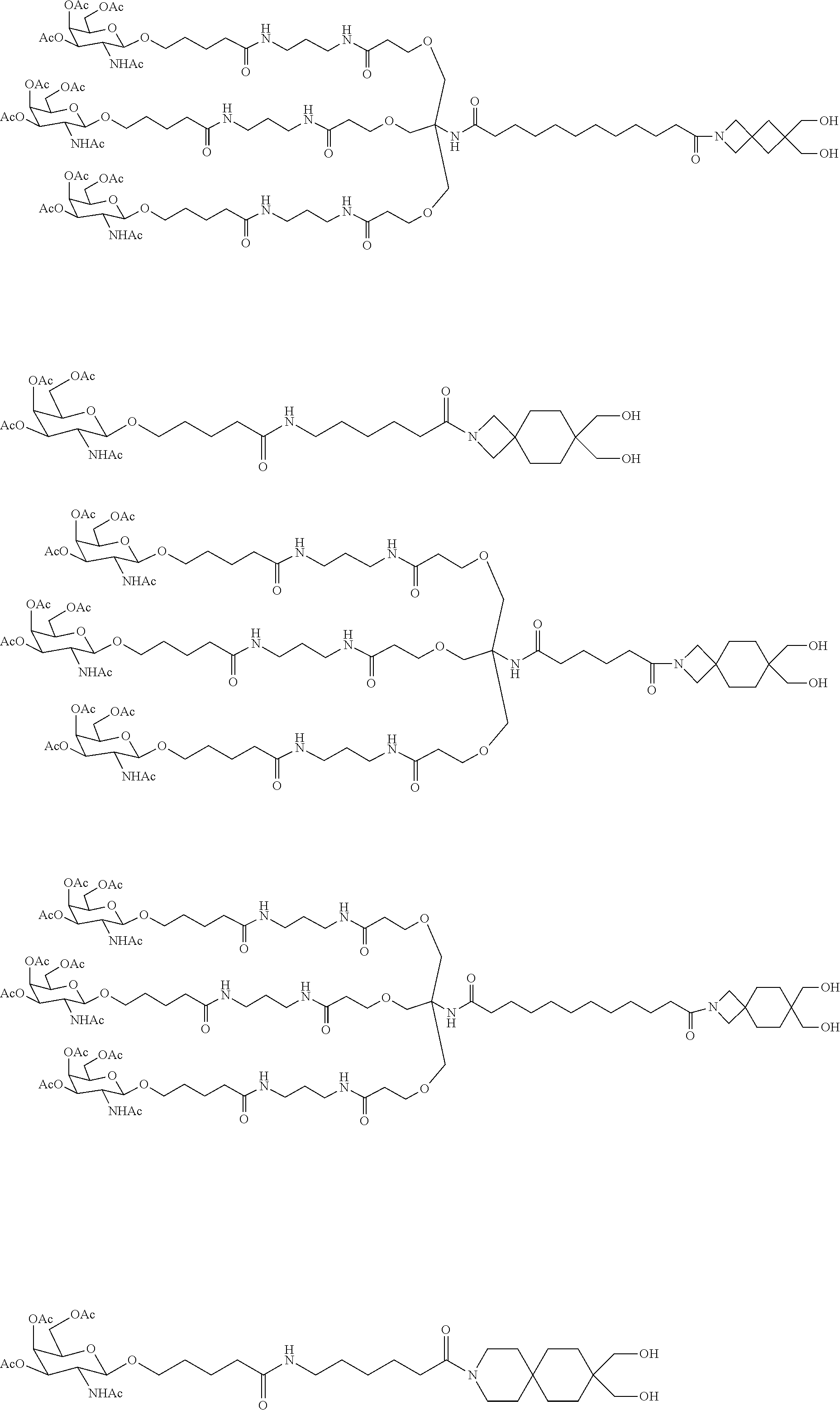 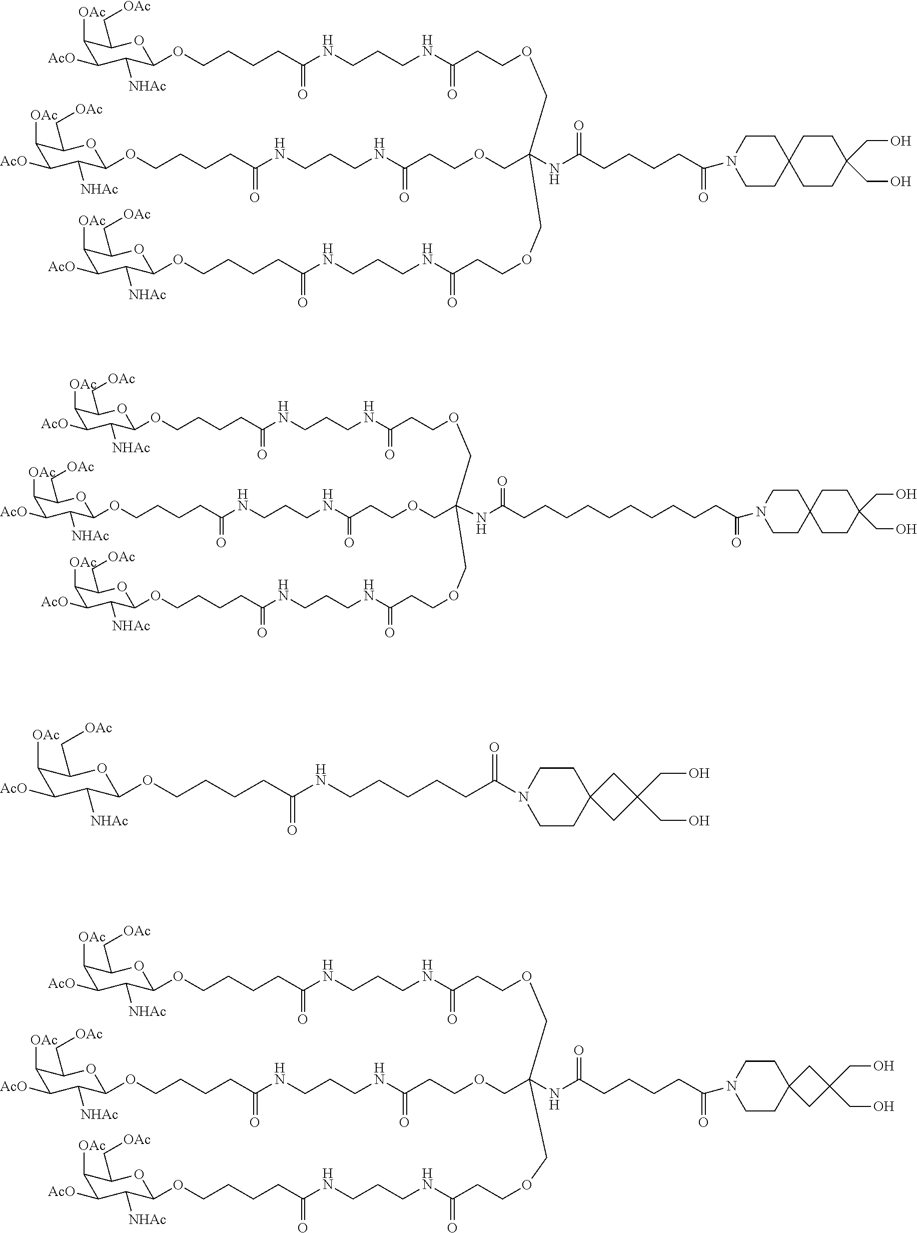 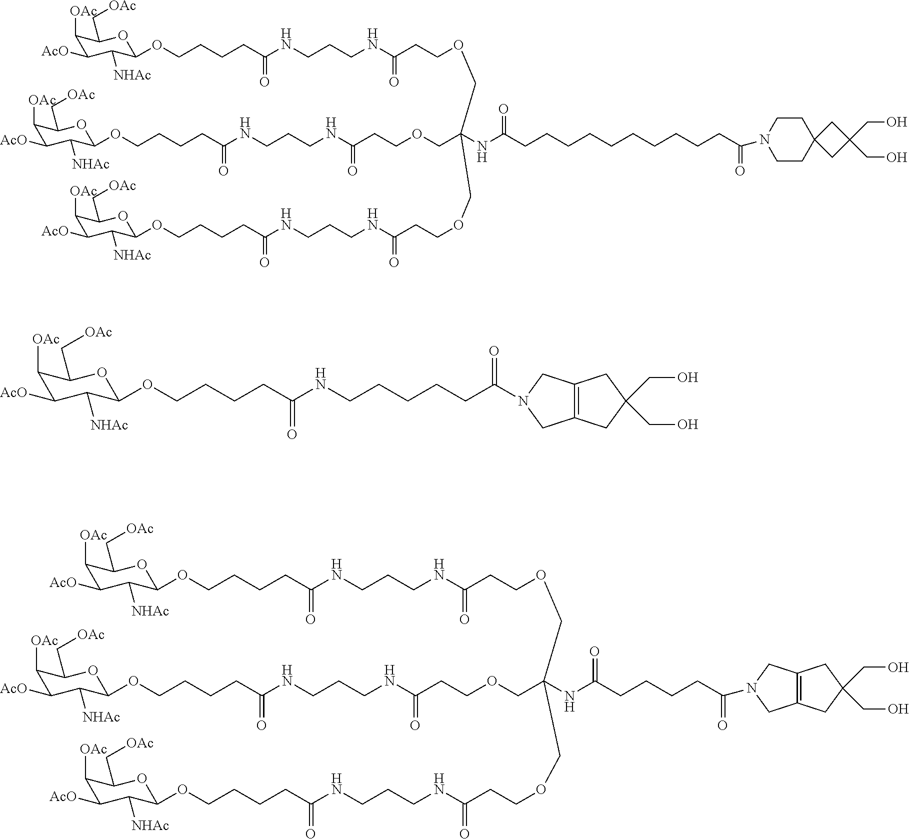 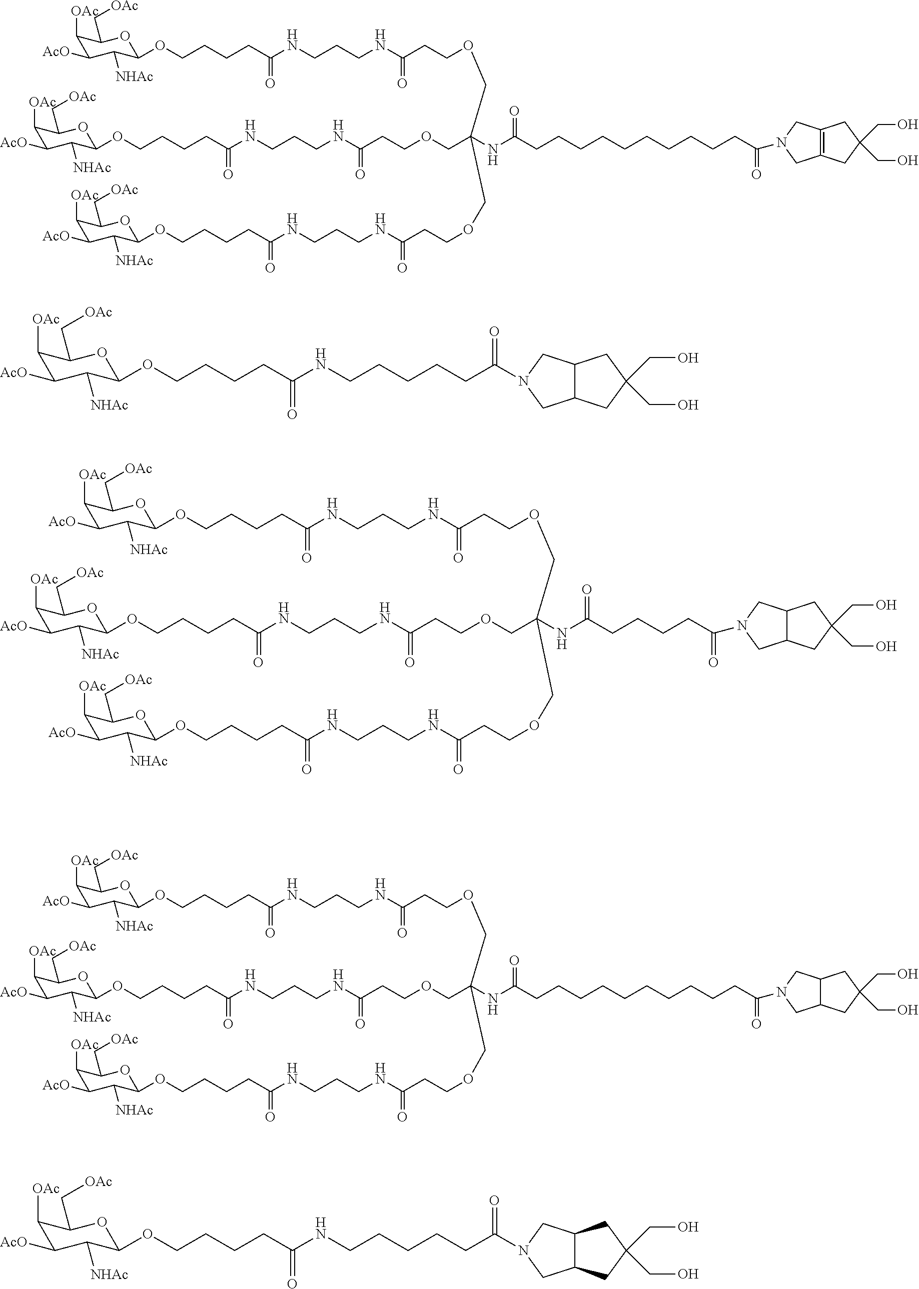 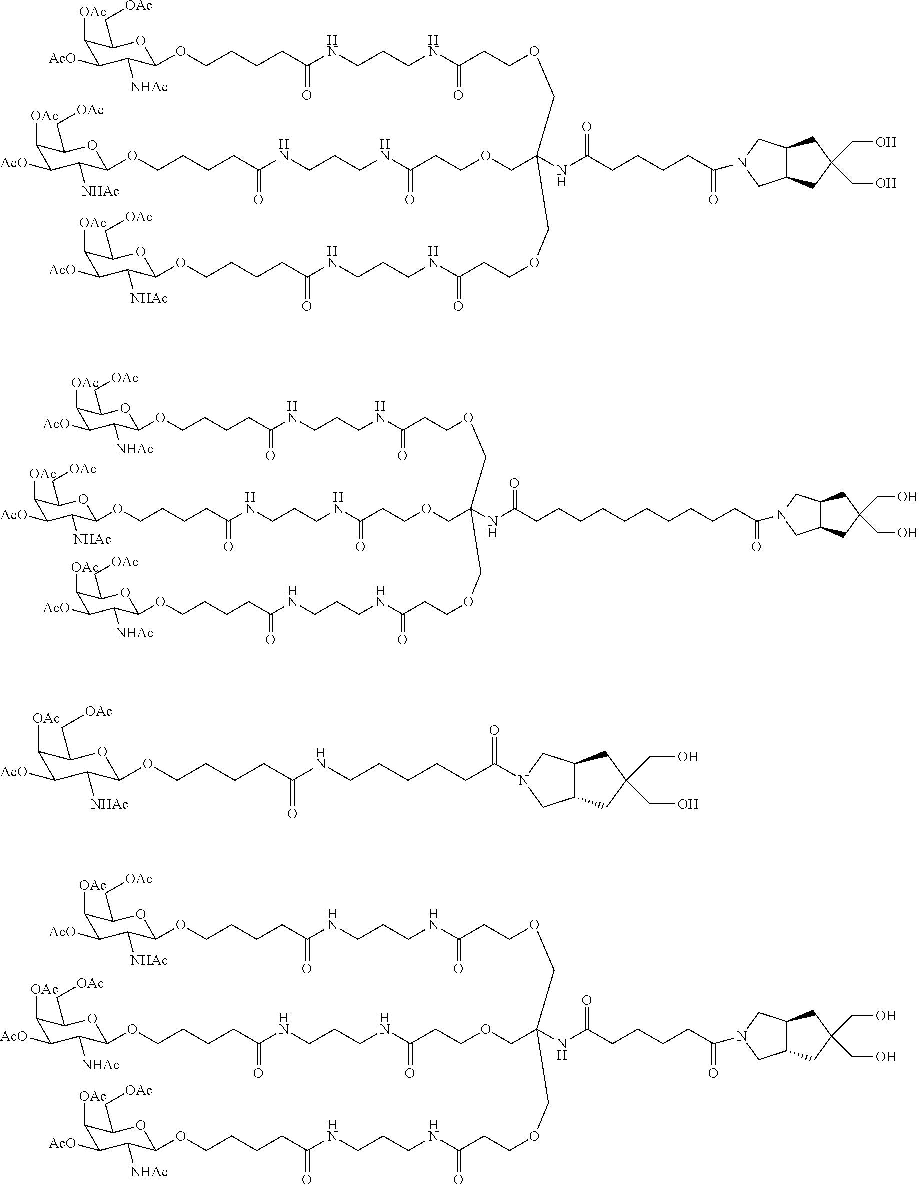 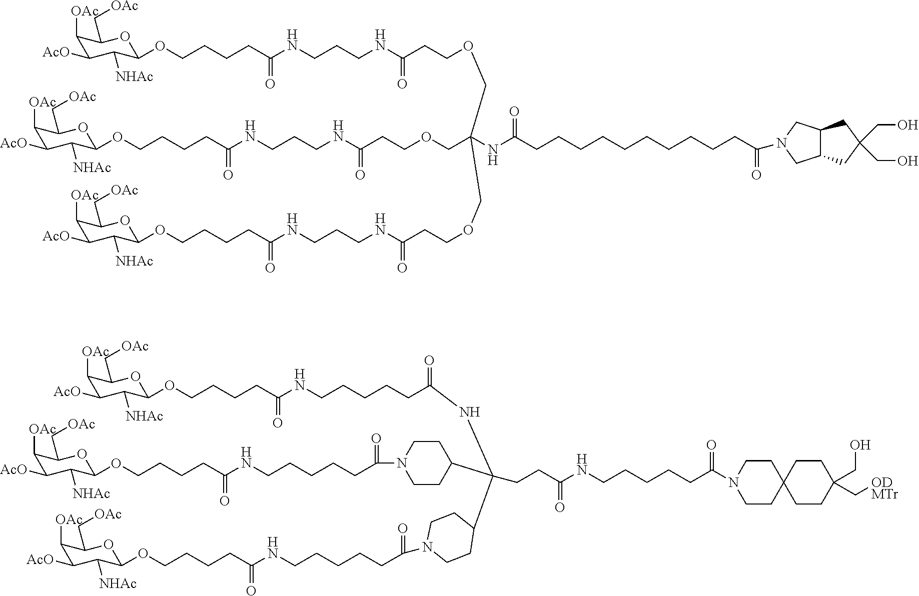 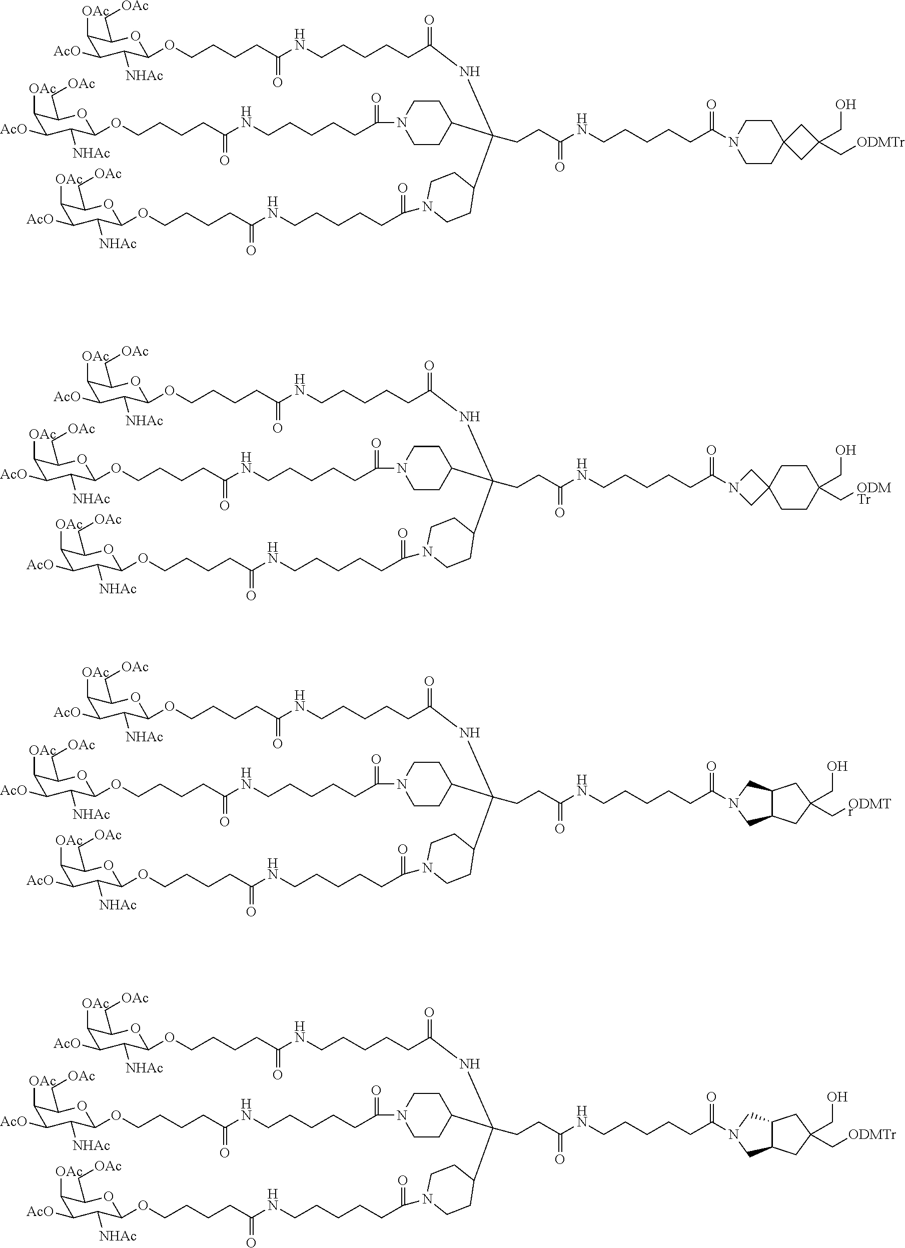 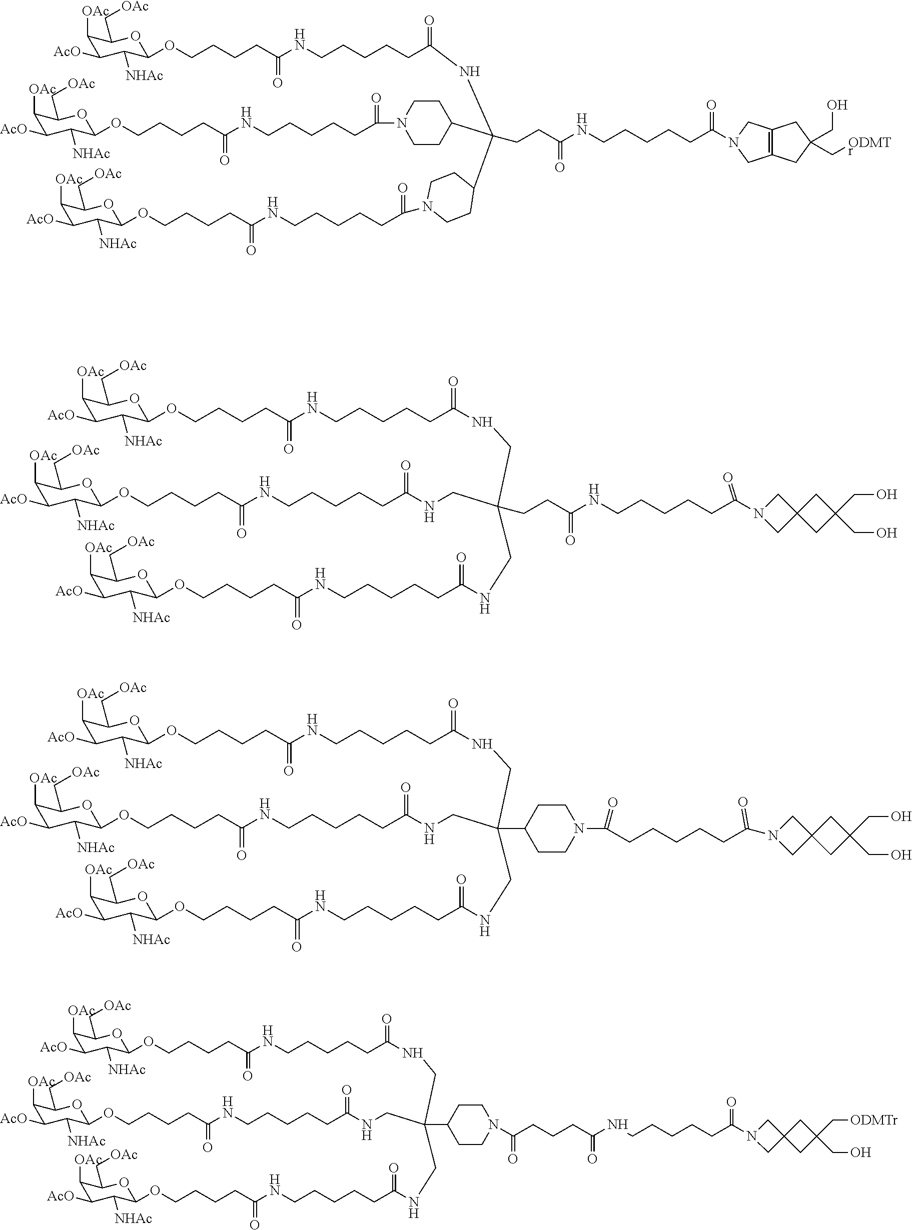 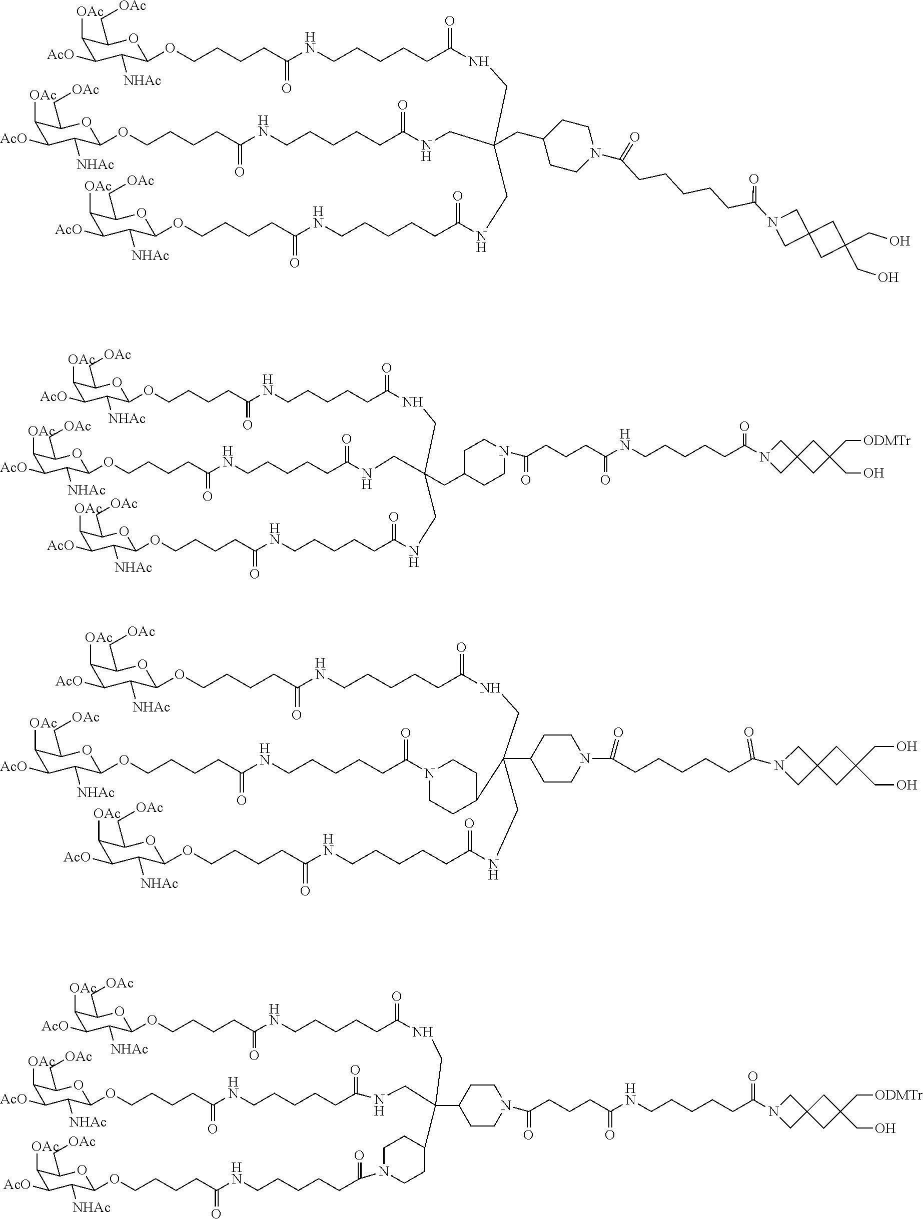 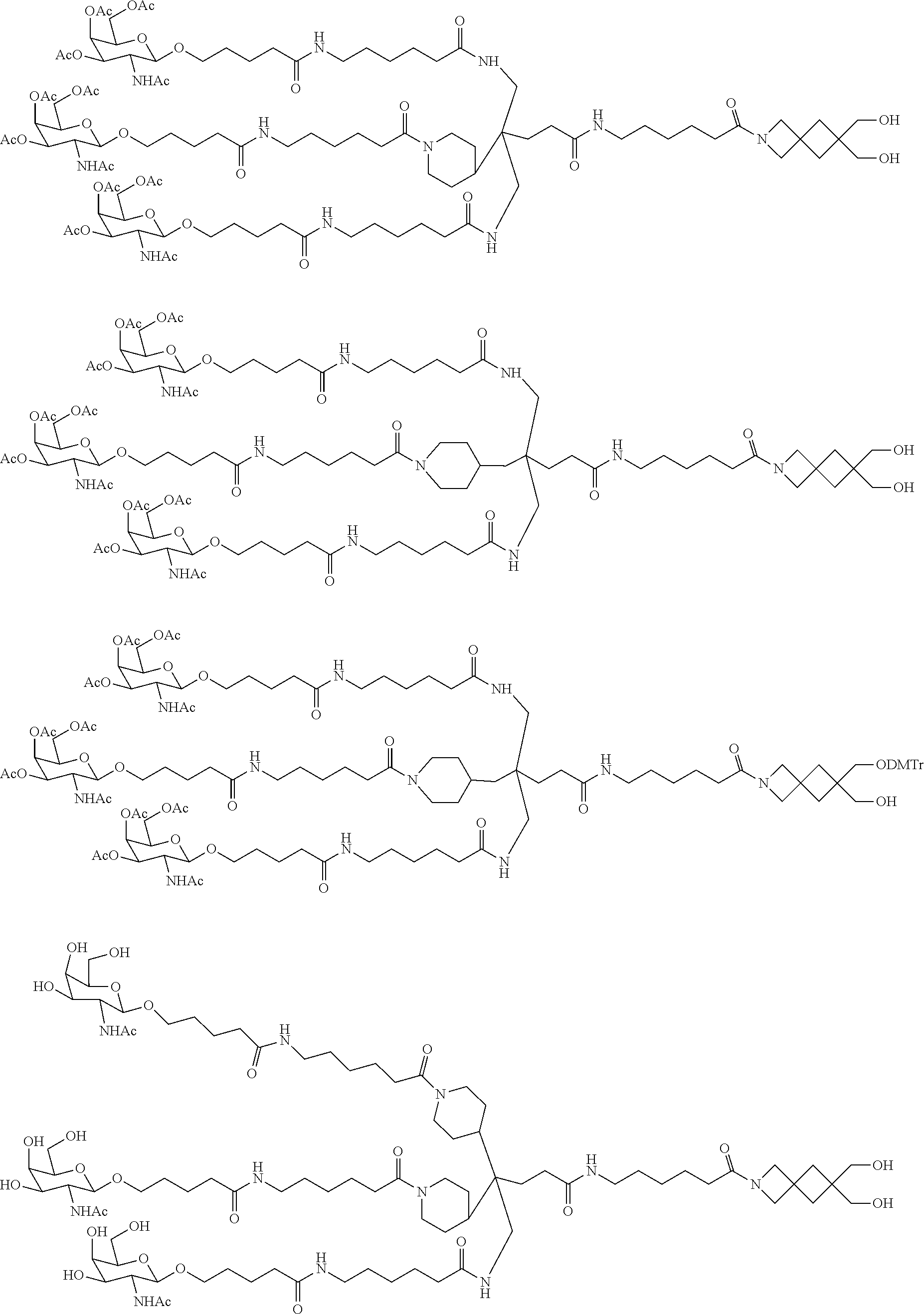 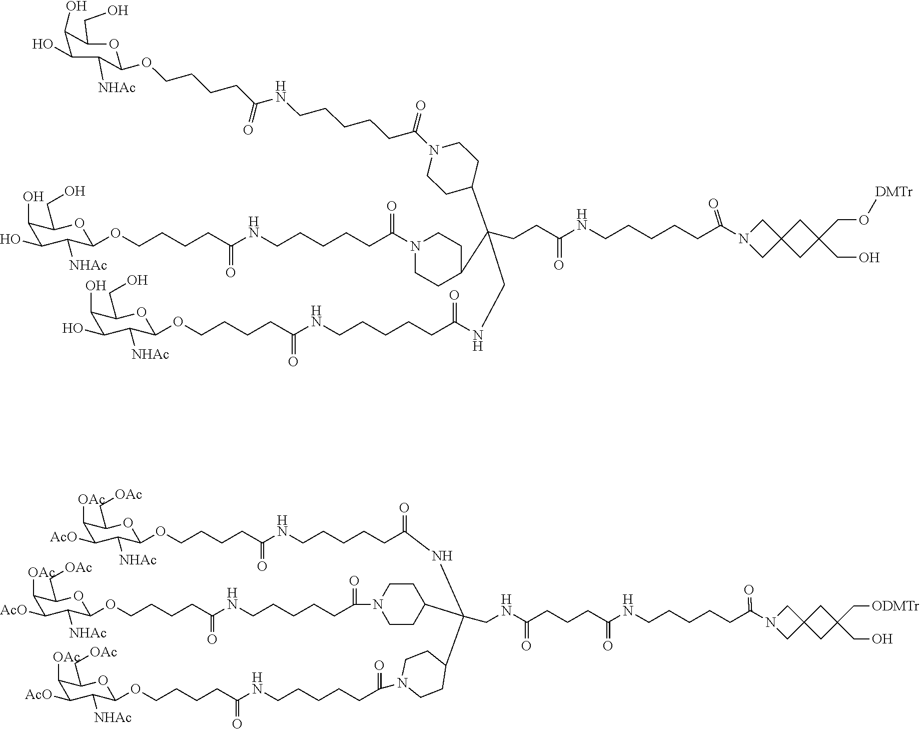 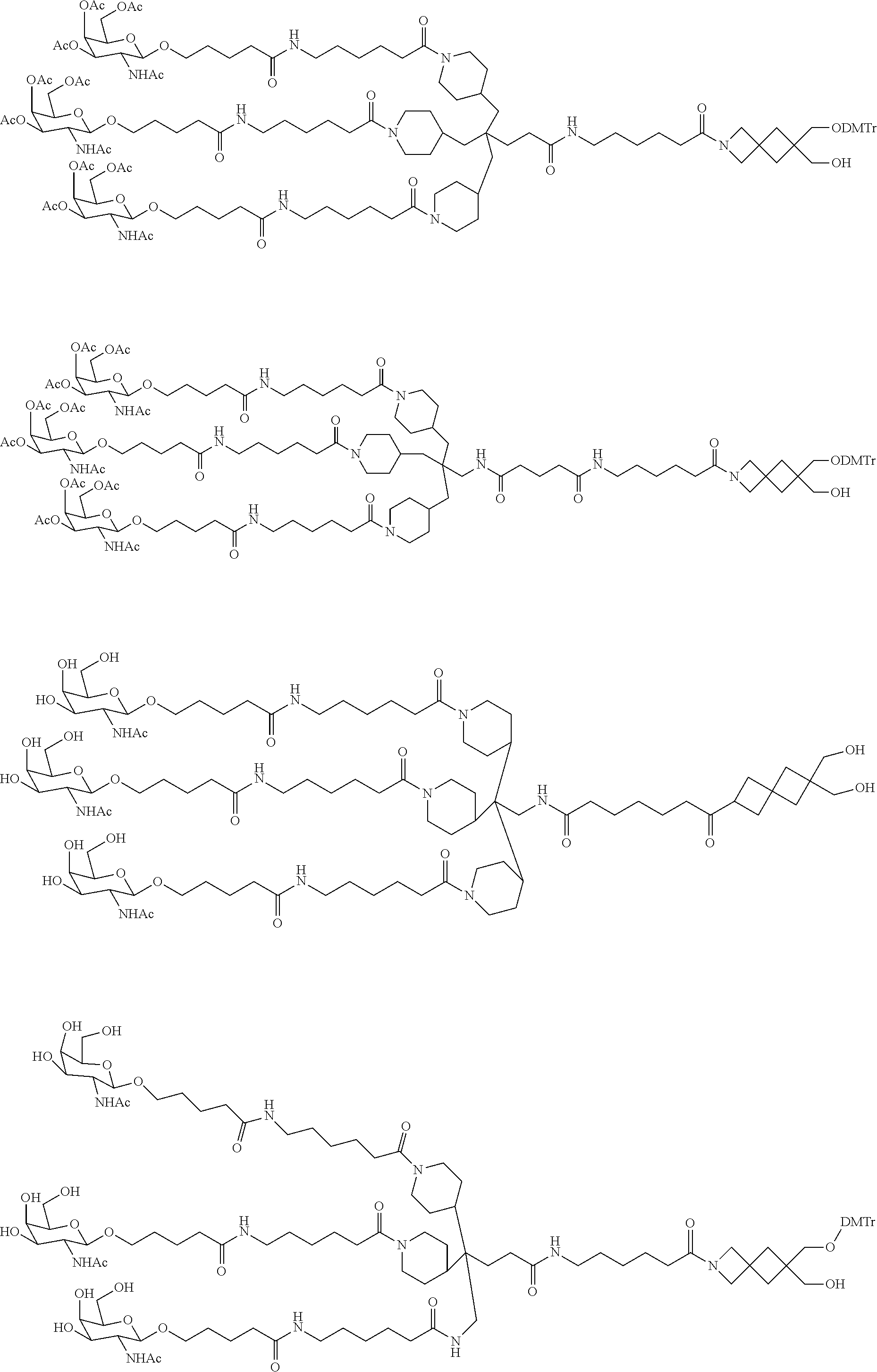 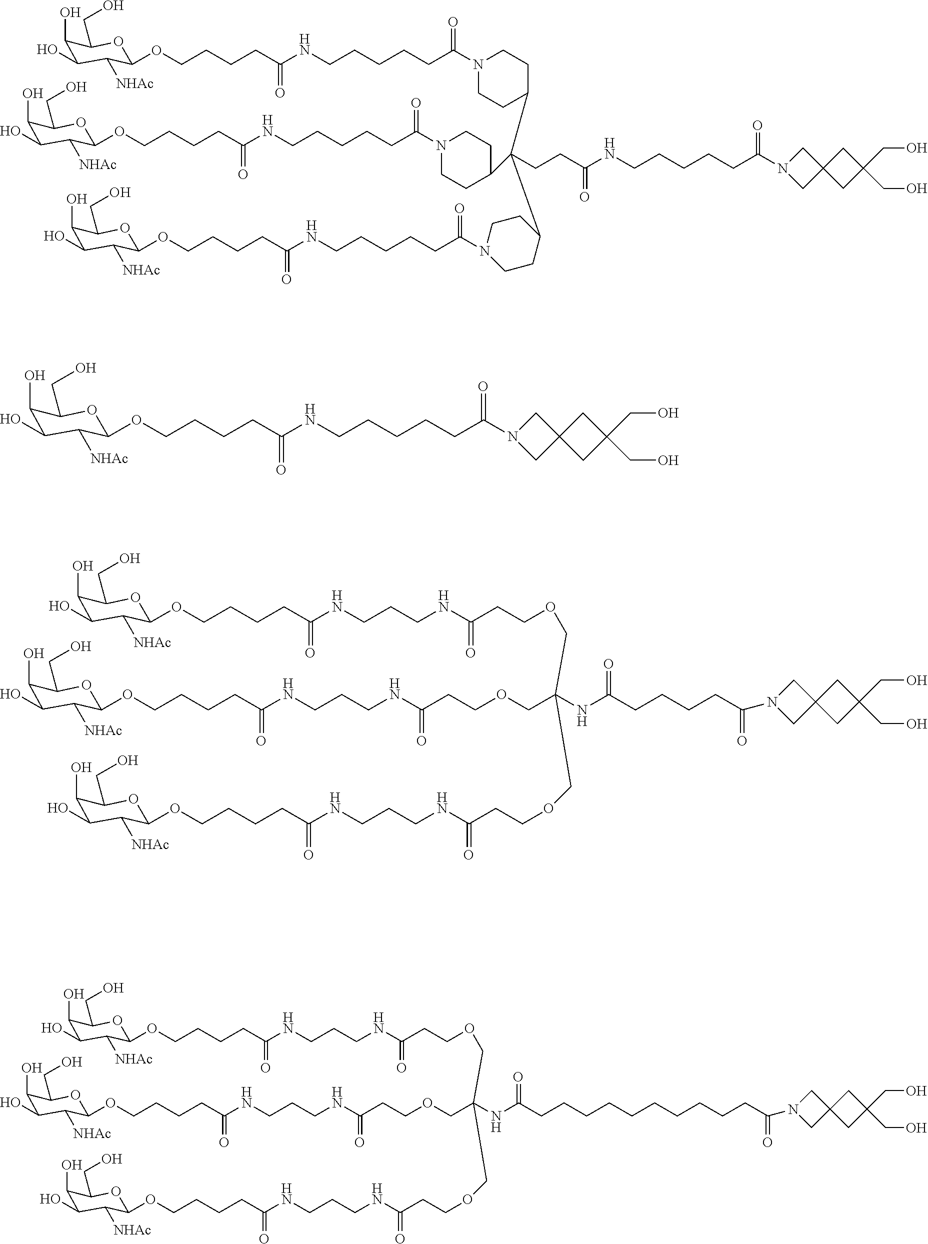 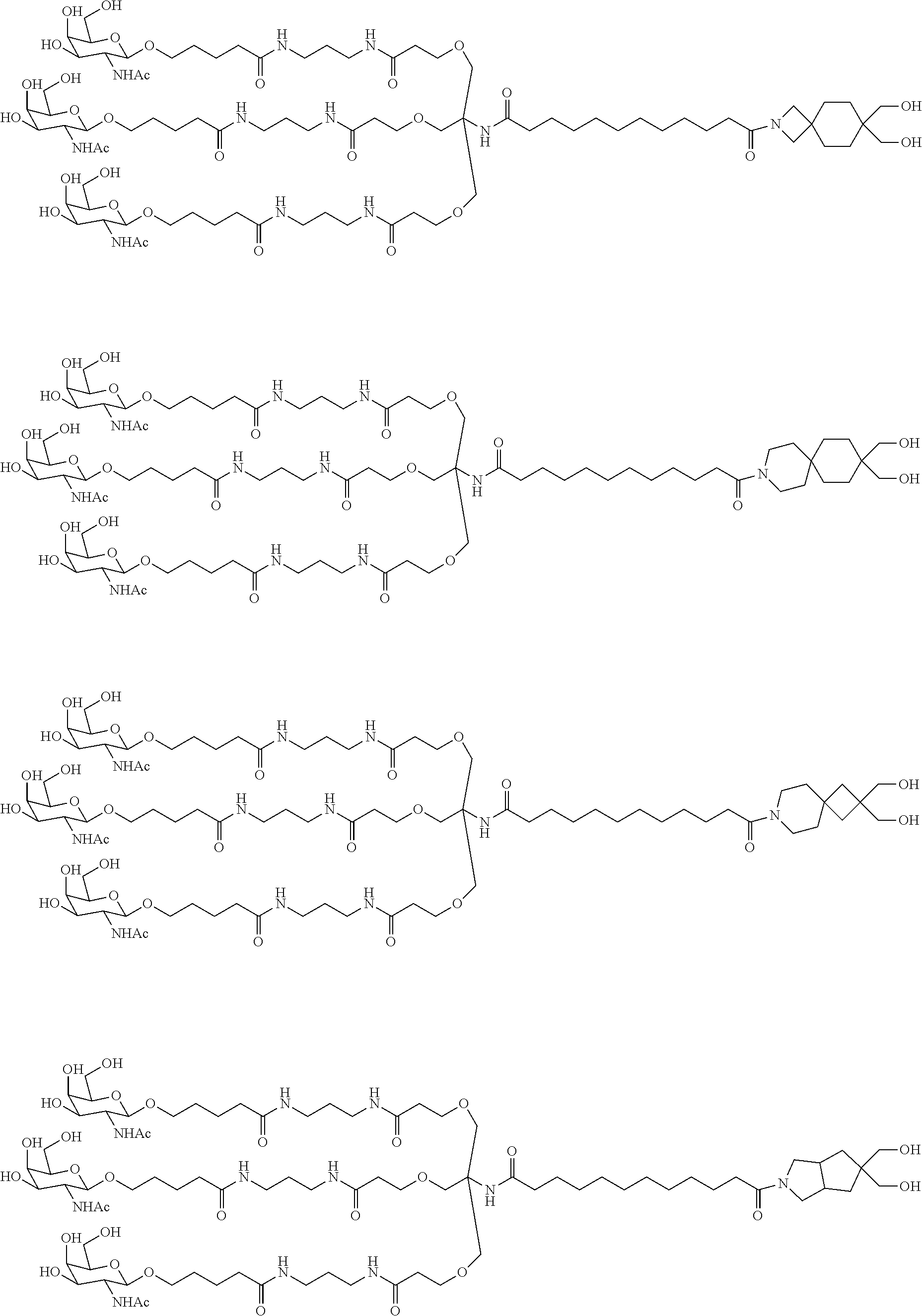 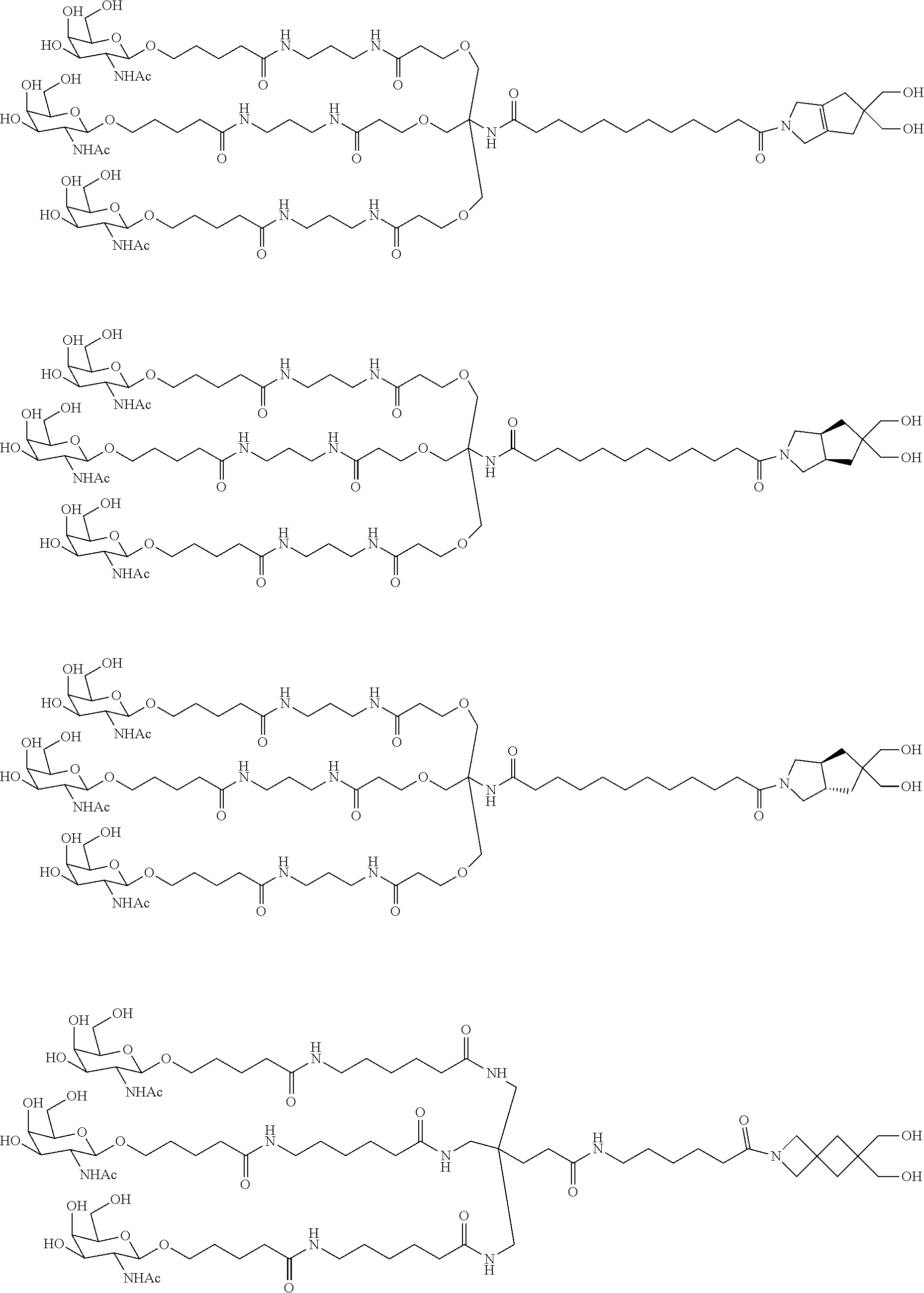 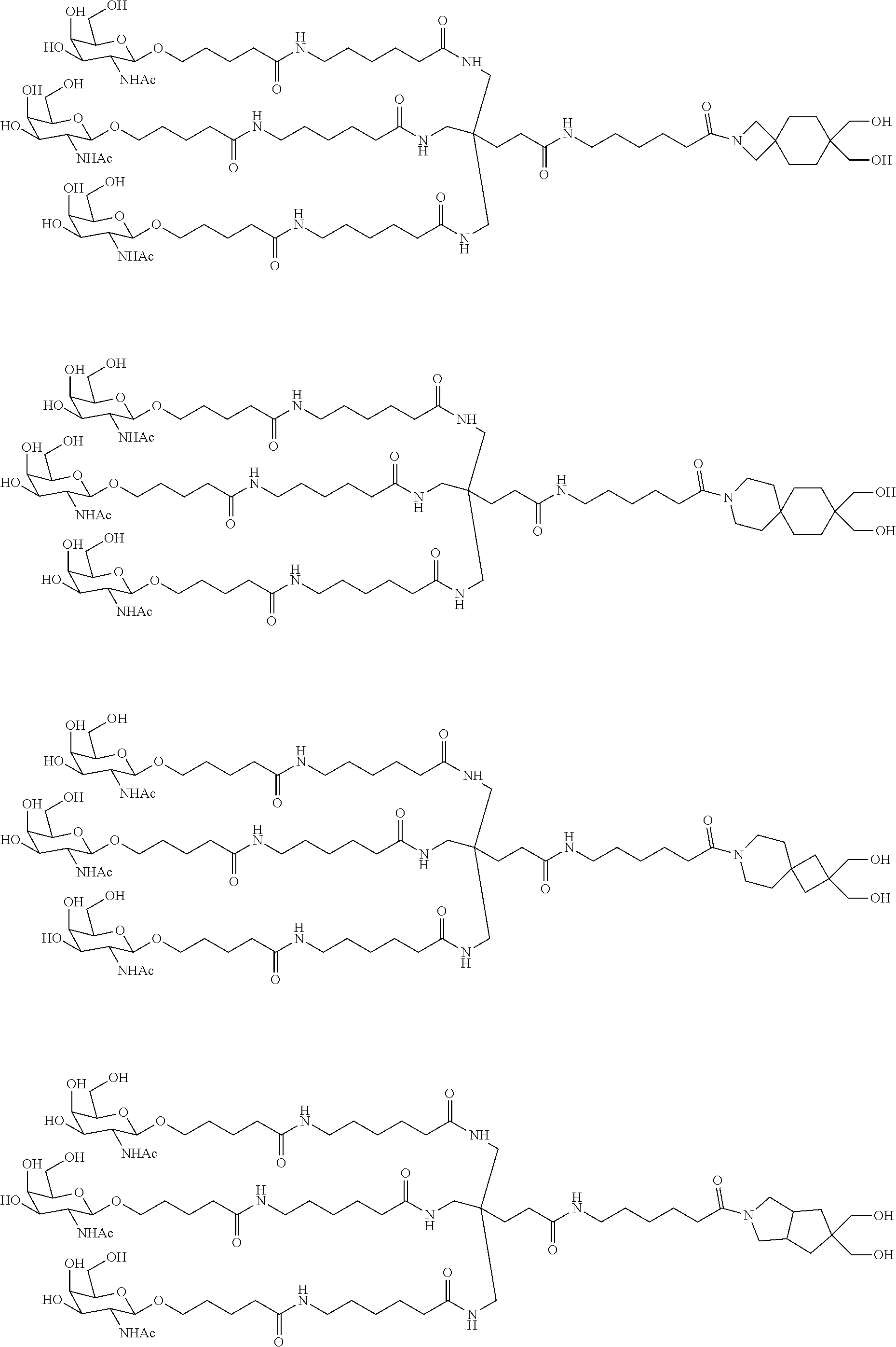 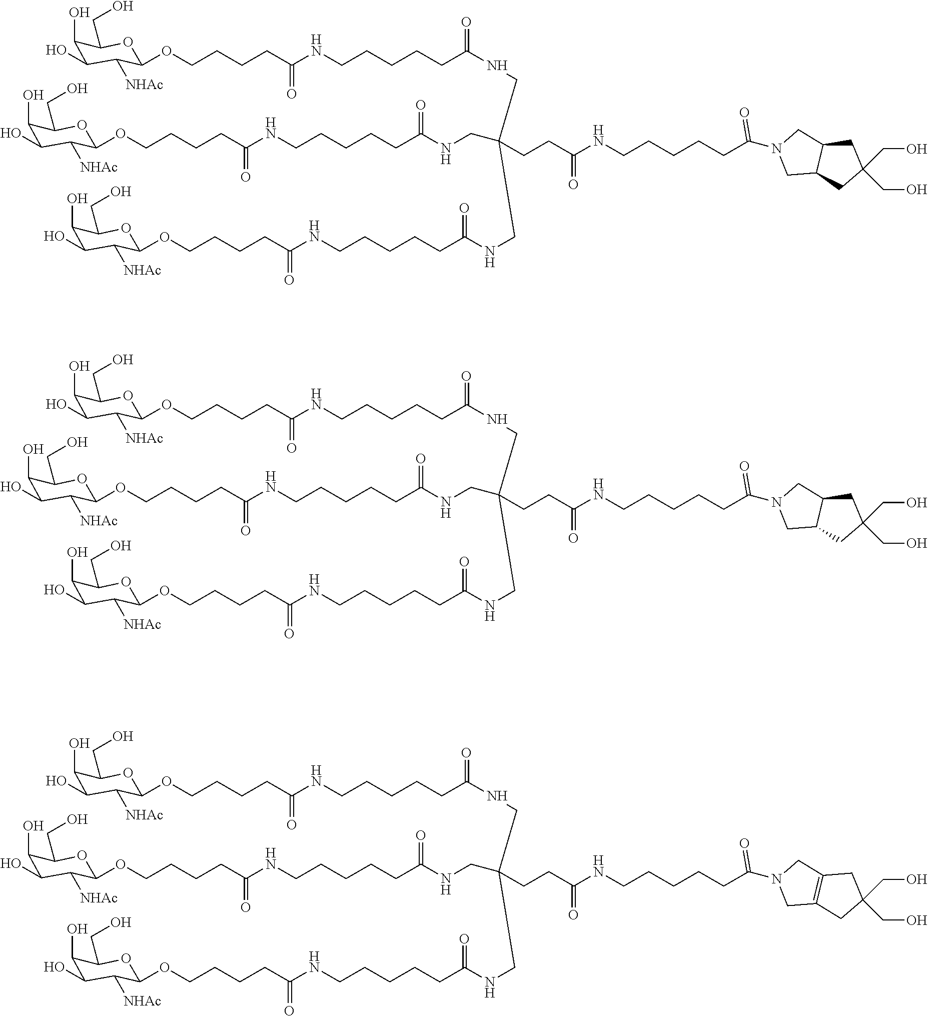 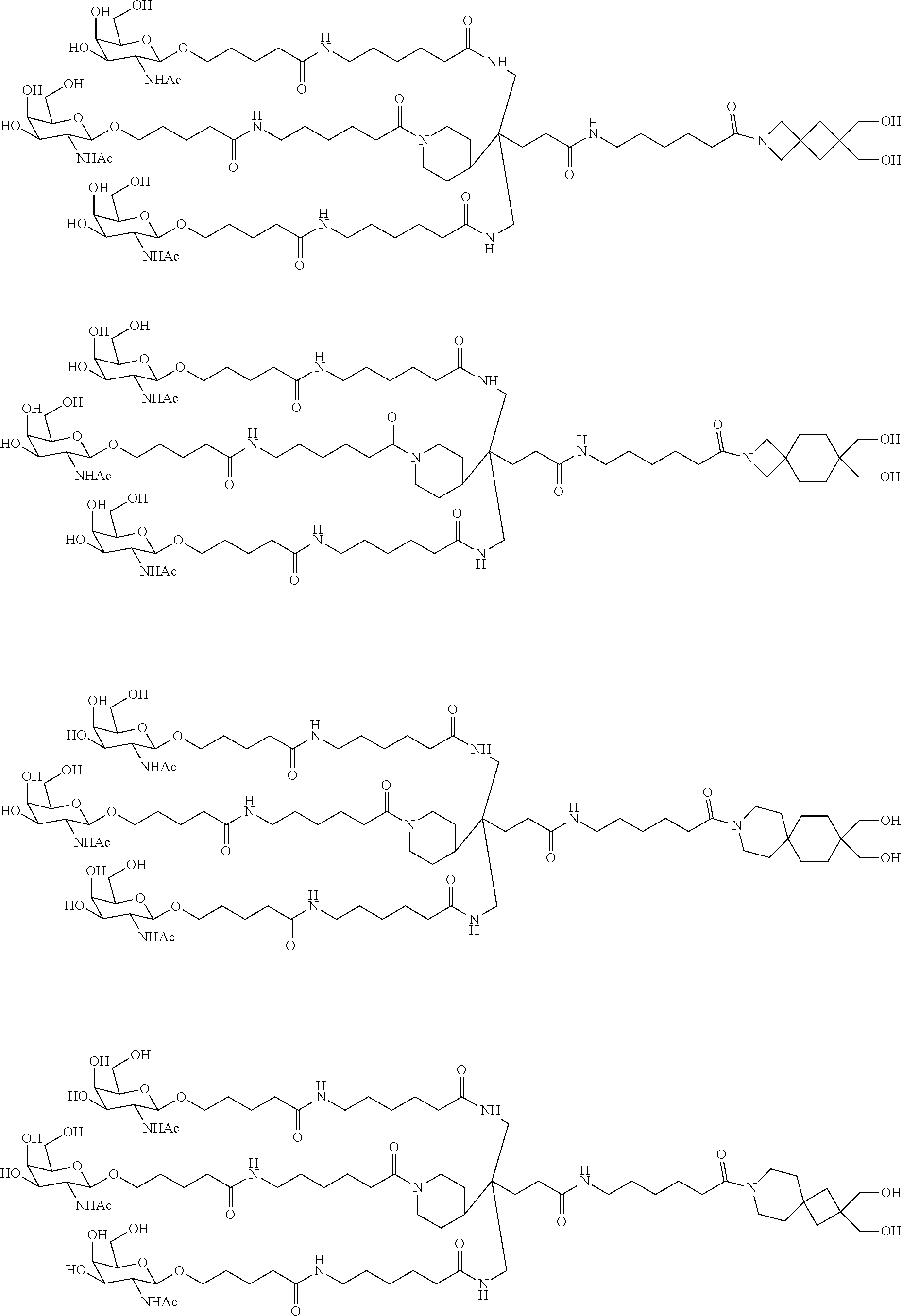 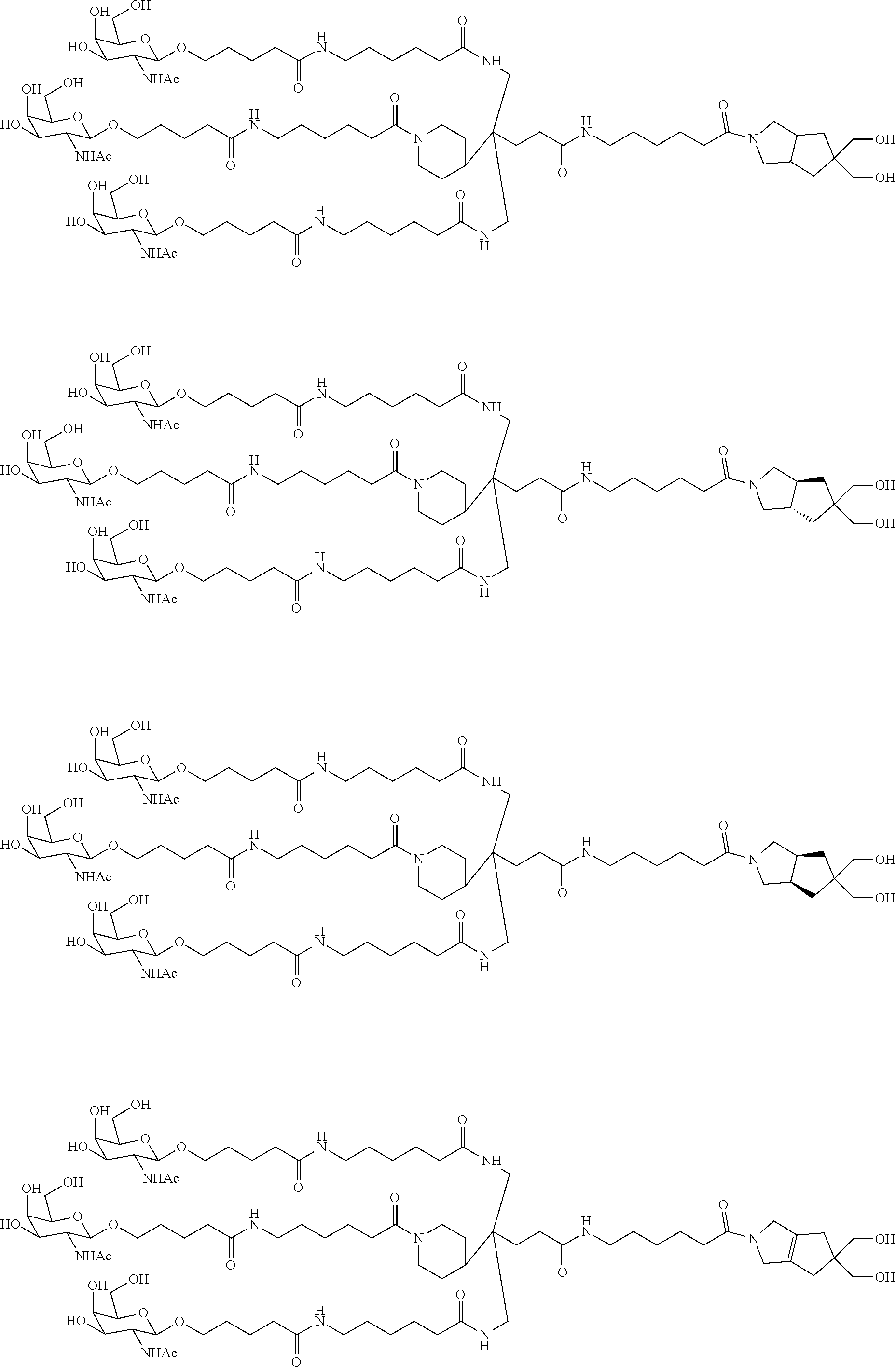 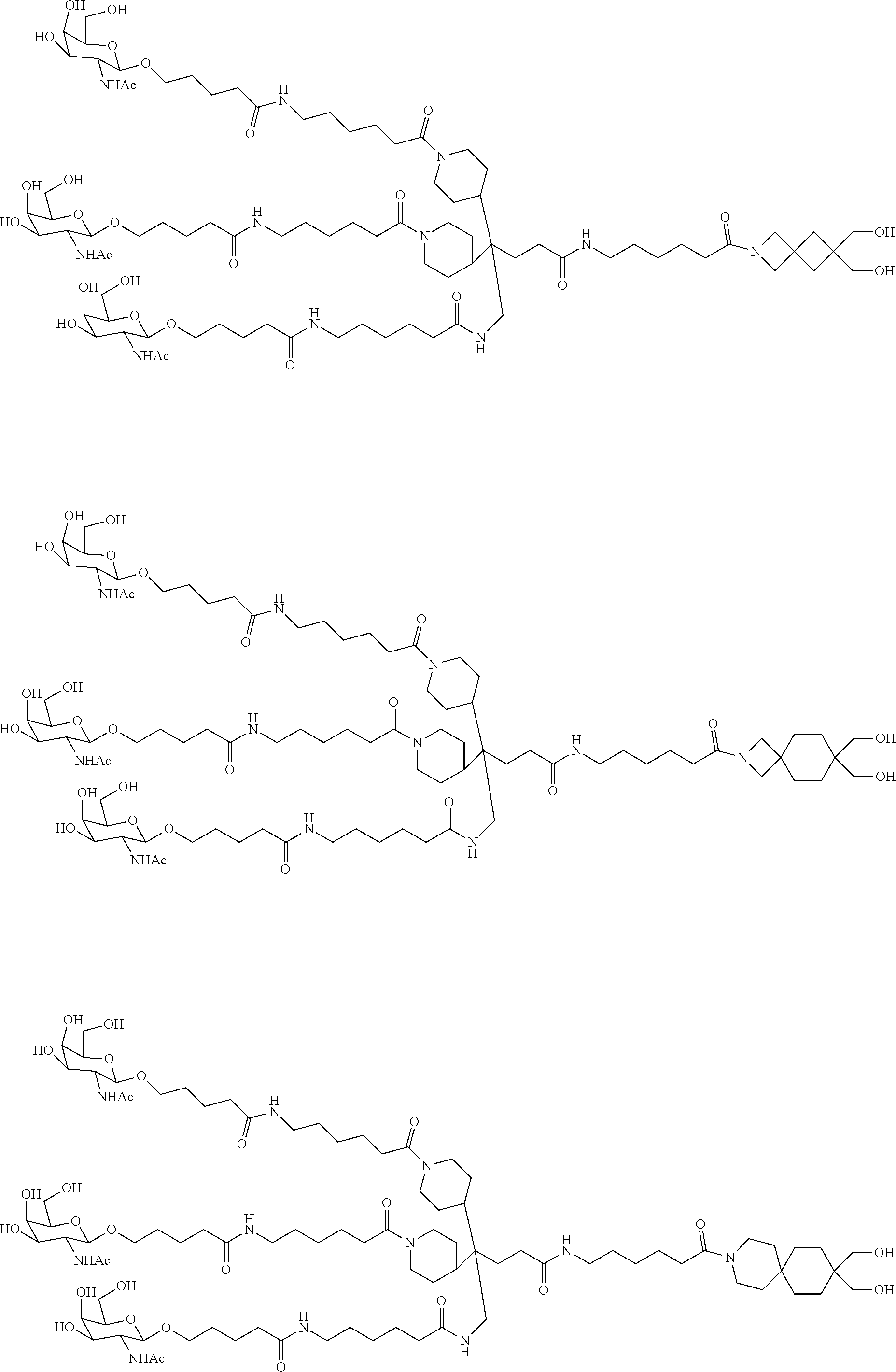 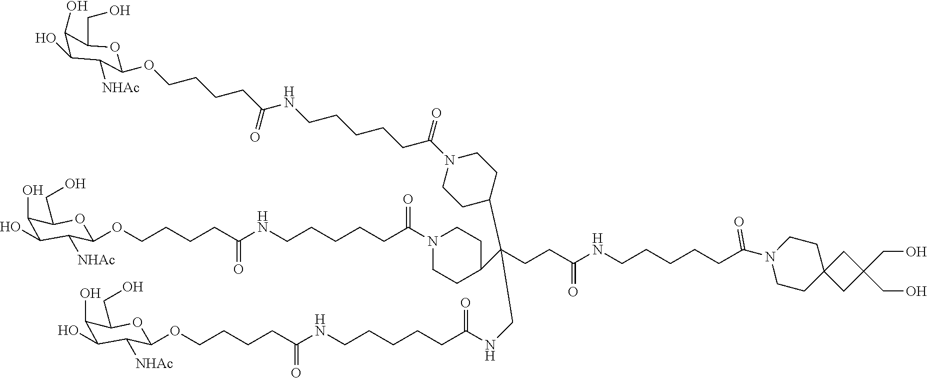 [SMILES: CC(=O)NC1C(OCCCCC(=O)NCCCCCC(=O)NCC(CCC(=O)NCCCCCC(=O)N2CCC3(CC2)CC(CO)(CO)C3)(C2CCN(C(=O)CCCCCNC(=O)CCCCOC3OC(CO)C(O)C(O)C3NC(C)=O)CC2)C2CCN(C(=O)CCCCCNC(=O)CCCCOC3OC(CO)C(O)C(O)C3NC(C)=O)CC2)OC(CO)C(O)C1O] 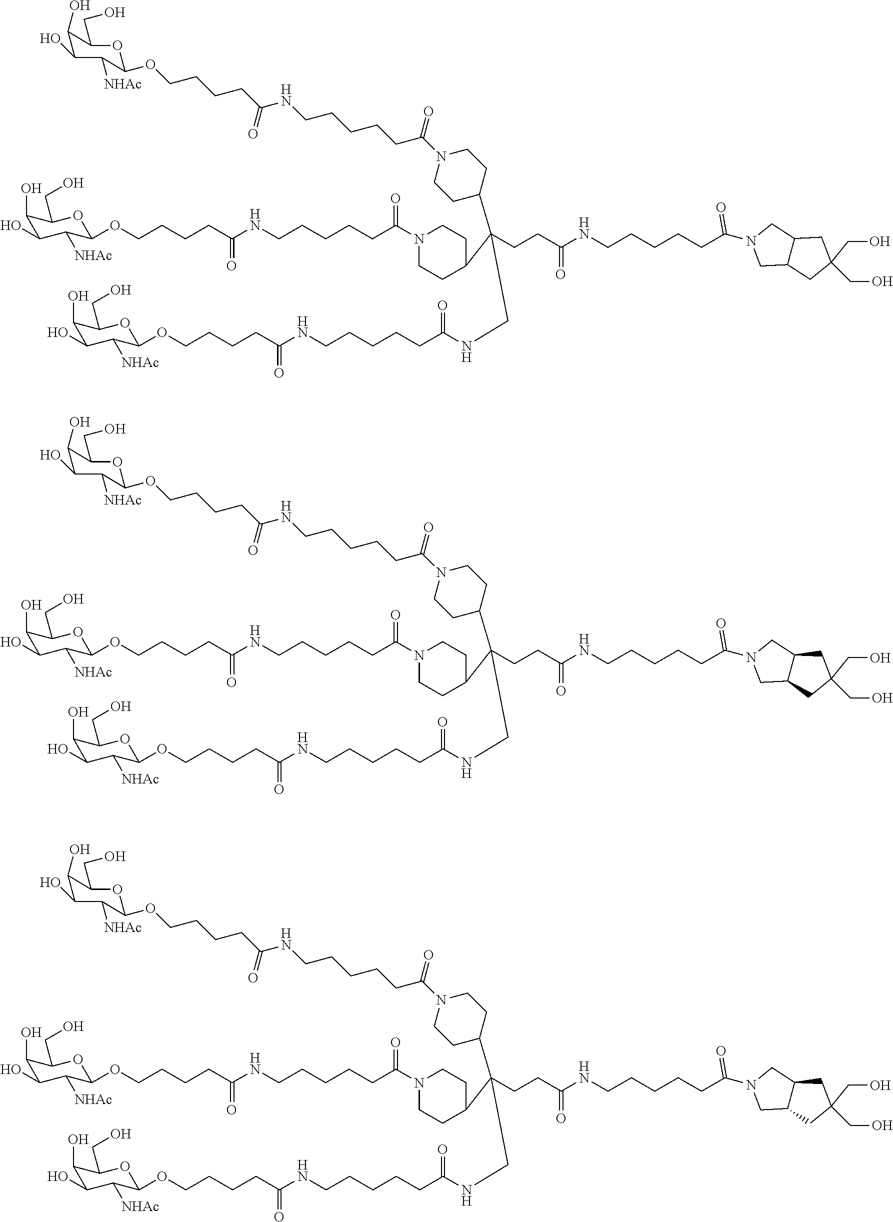 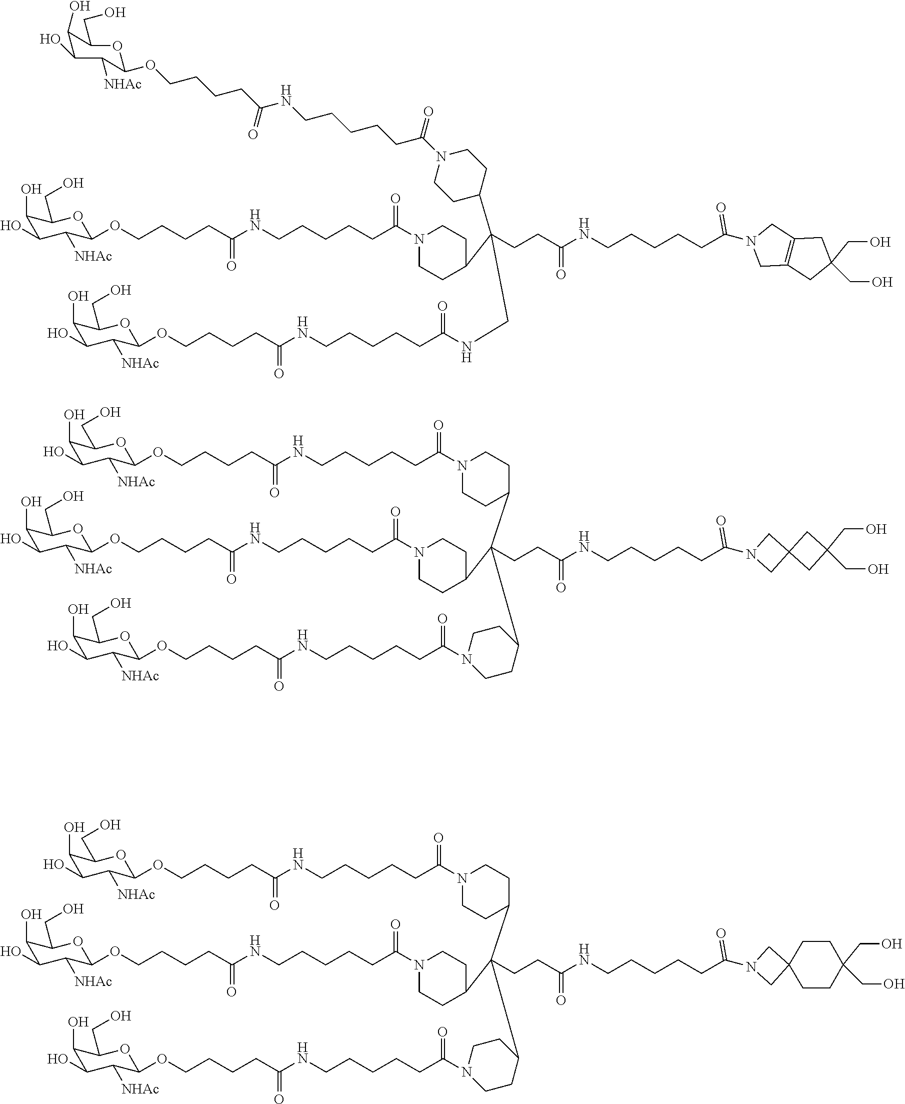 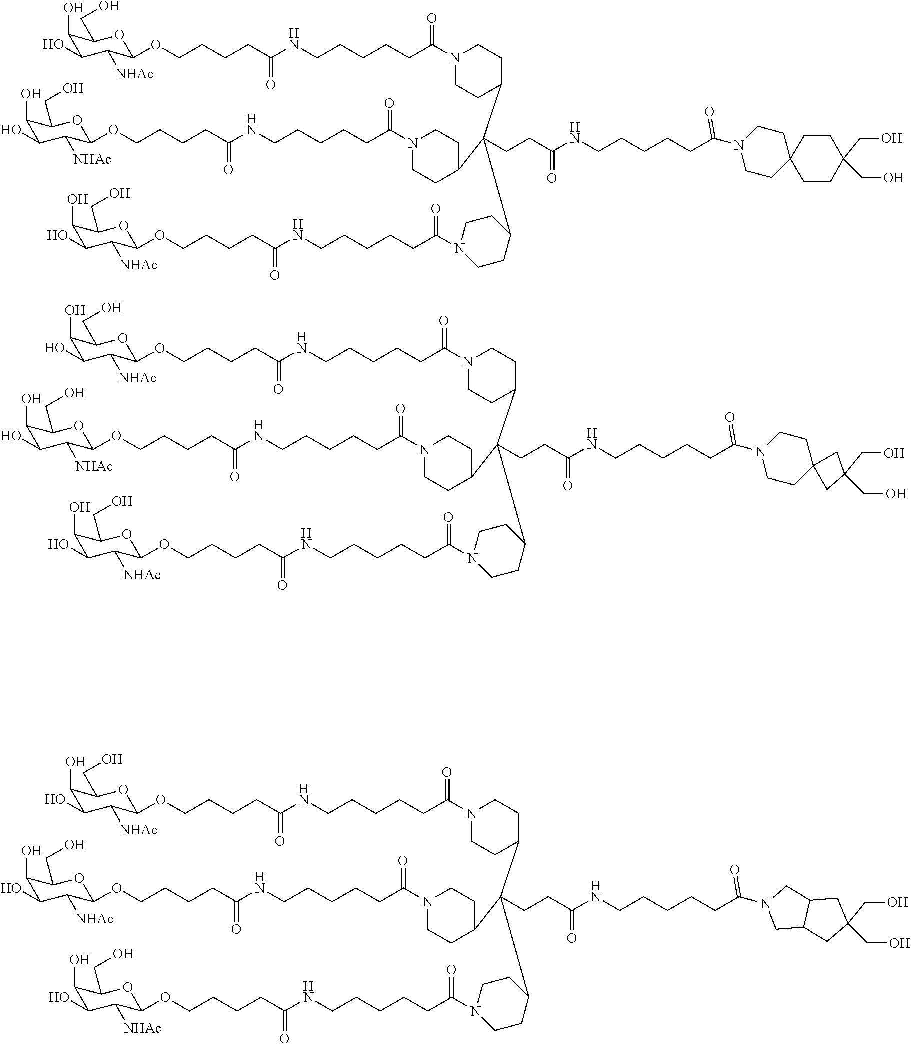 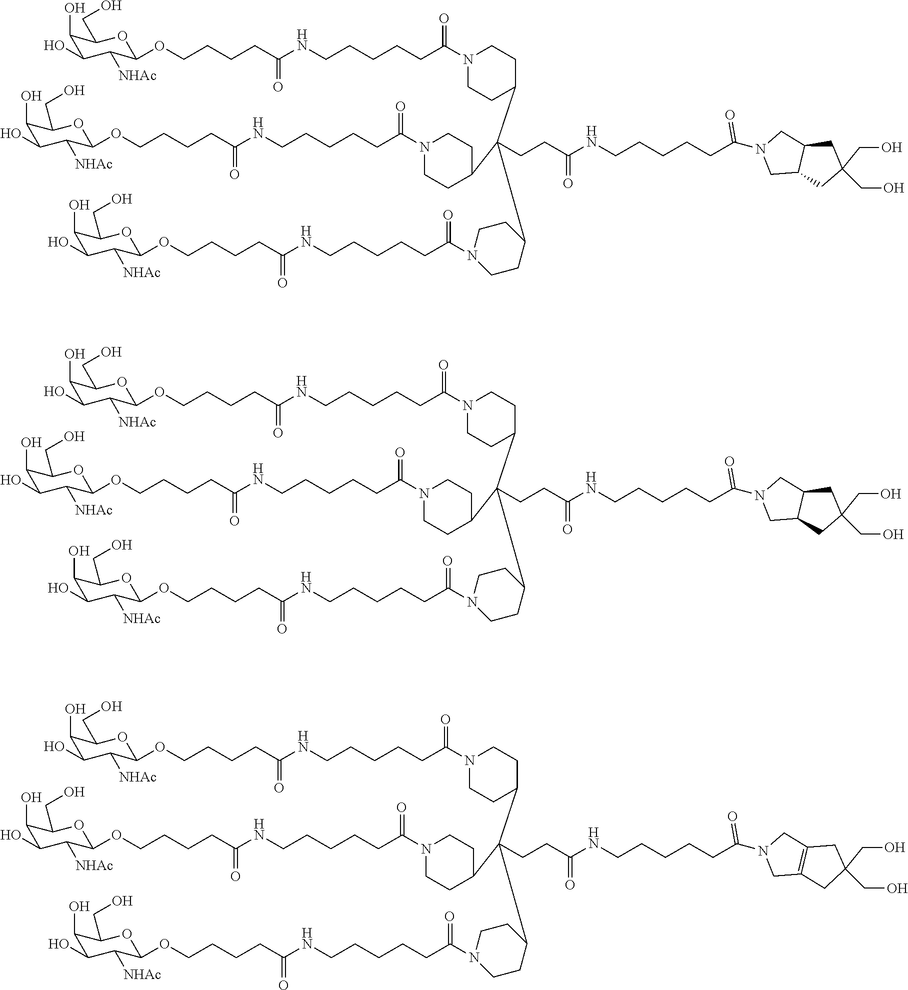 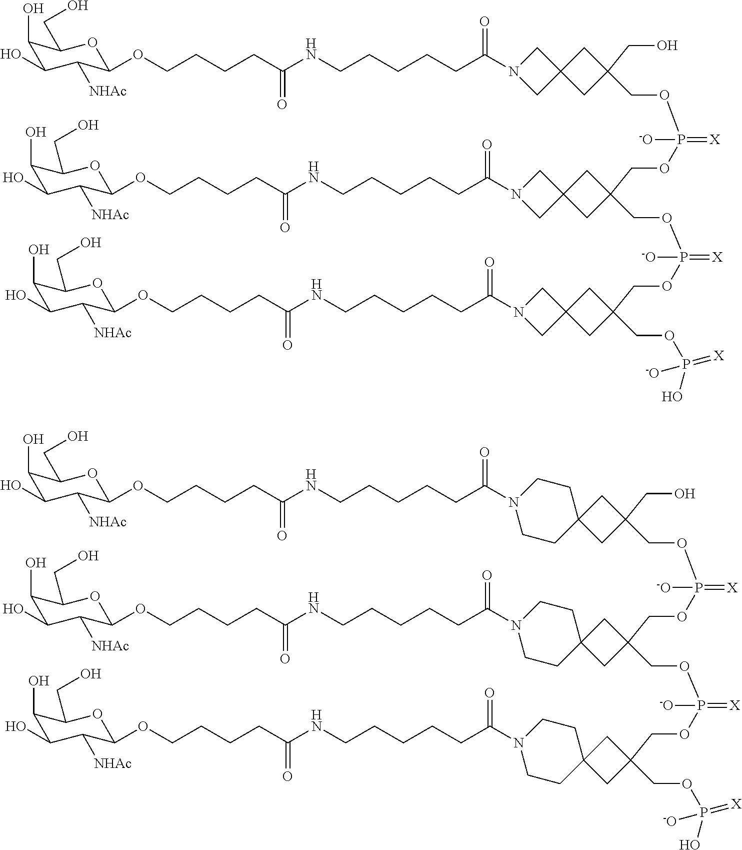 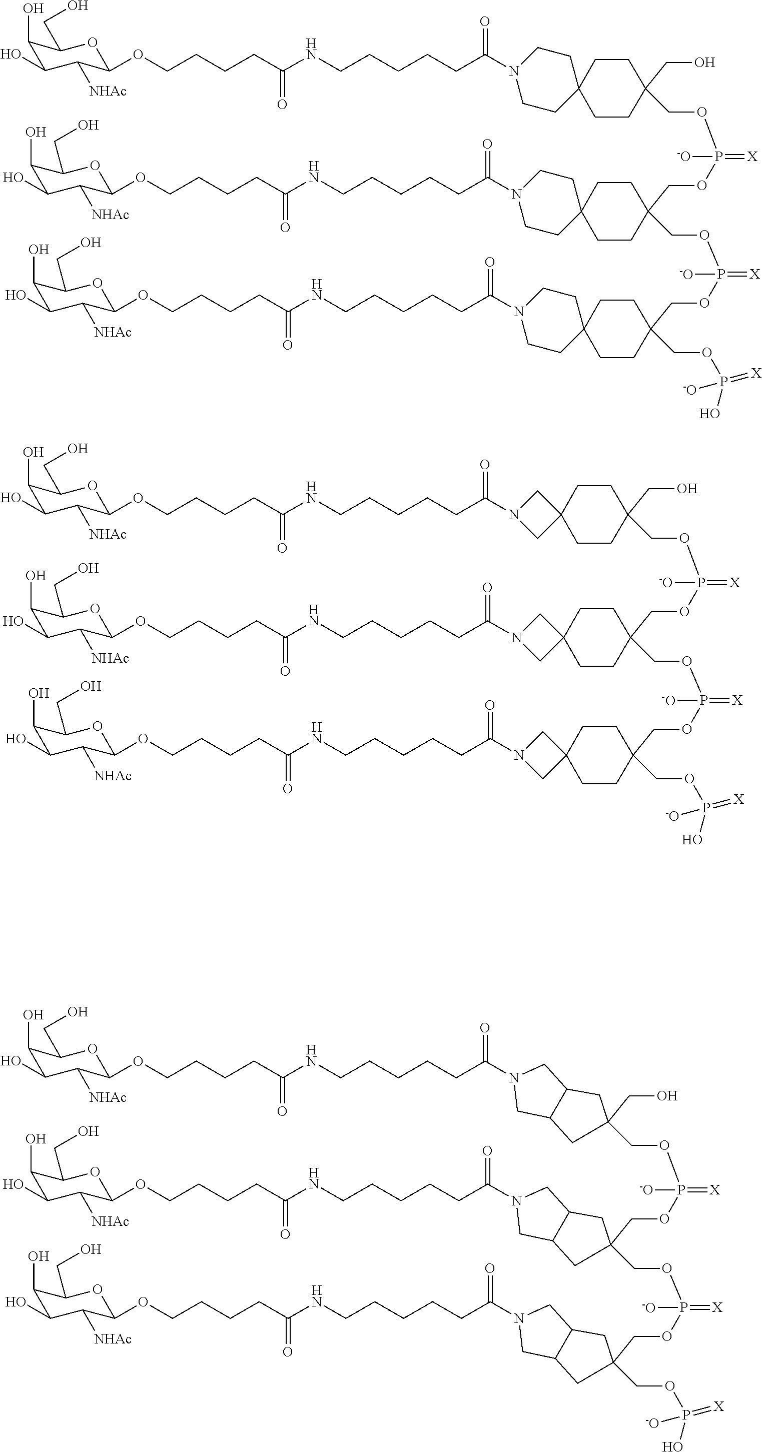 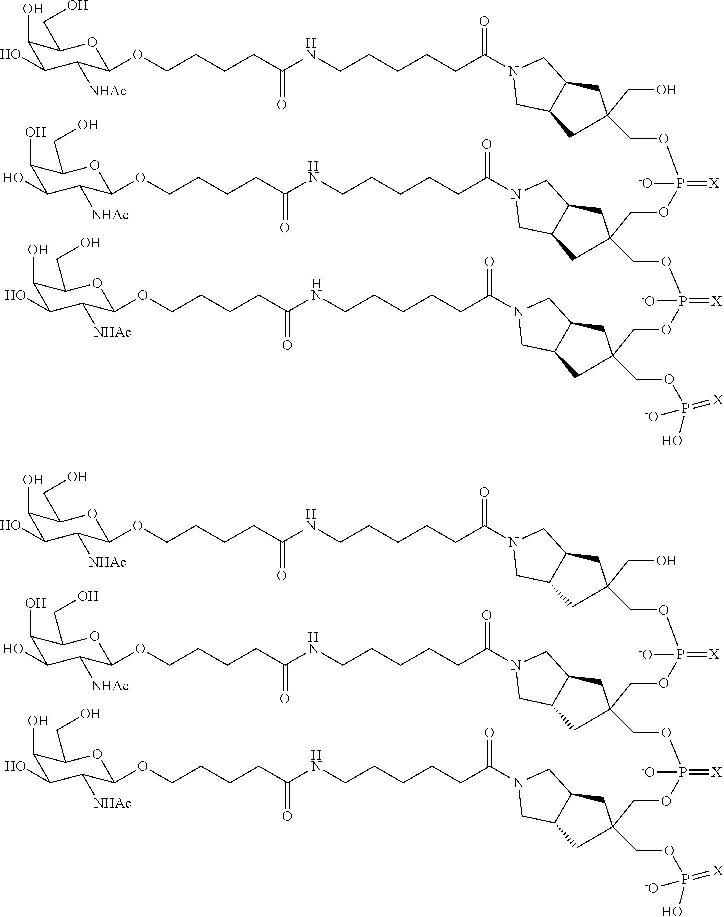 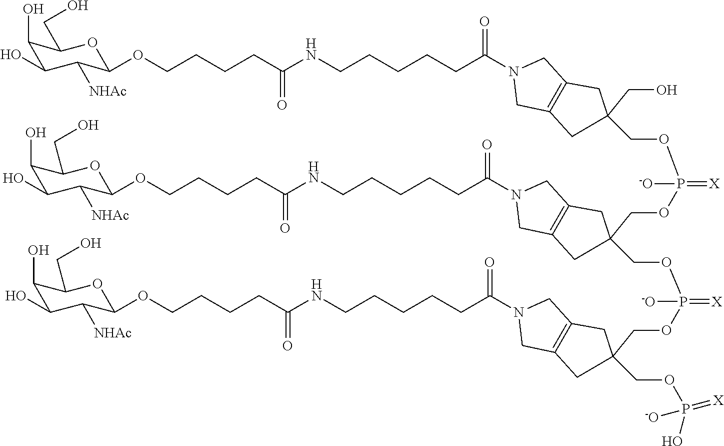
X is O or S.

13. A targeting ligand of claim 1, linked to a therapeutic agent, for use as medicament.

14. A therapeutic compound or pharmaceutically acceptable salt thereof, comprising at least one therapeutic oligonucleotide, and the oligonucleotide conjugate to the targeting ligand of claim 1.

15. The therapeutic compound or pharmaceutically acceptable salt thereof of claim 14, wherein the therapeutic compound is: 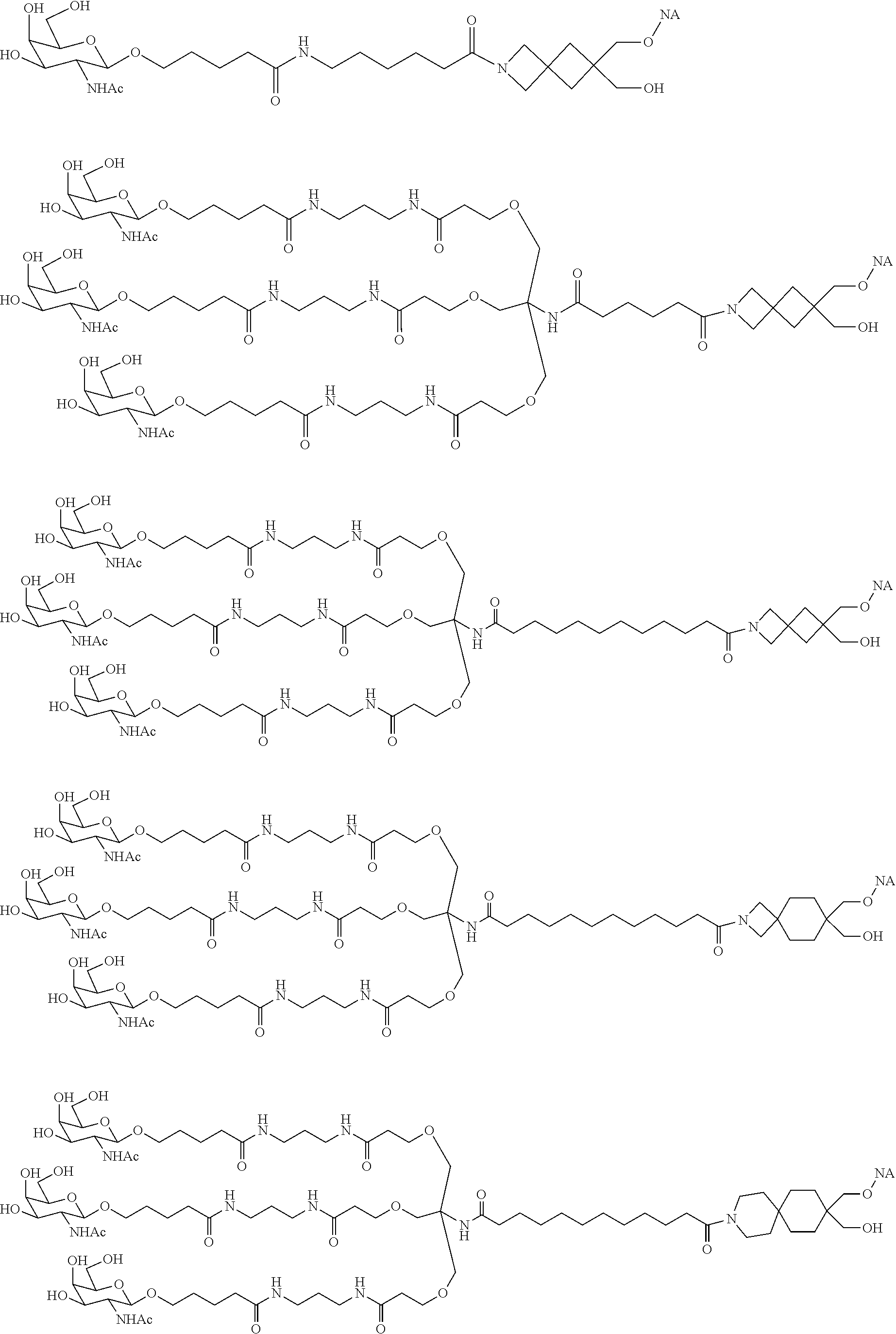 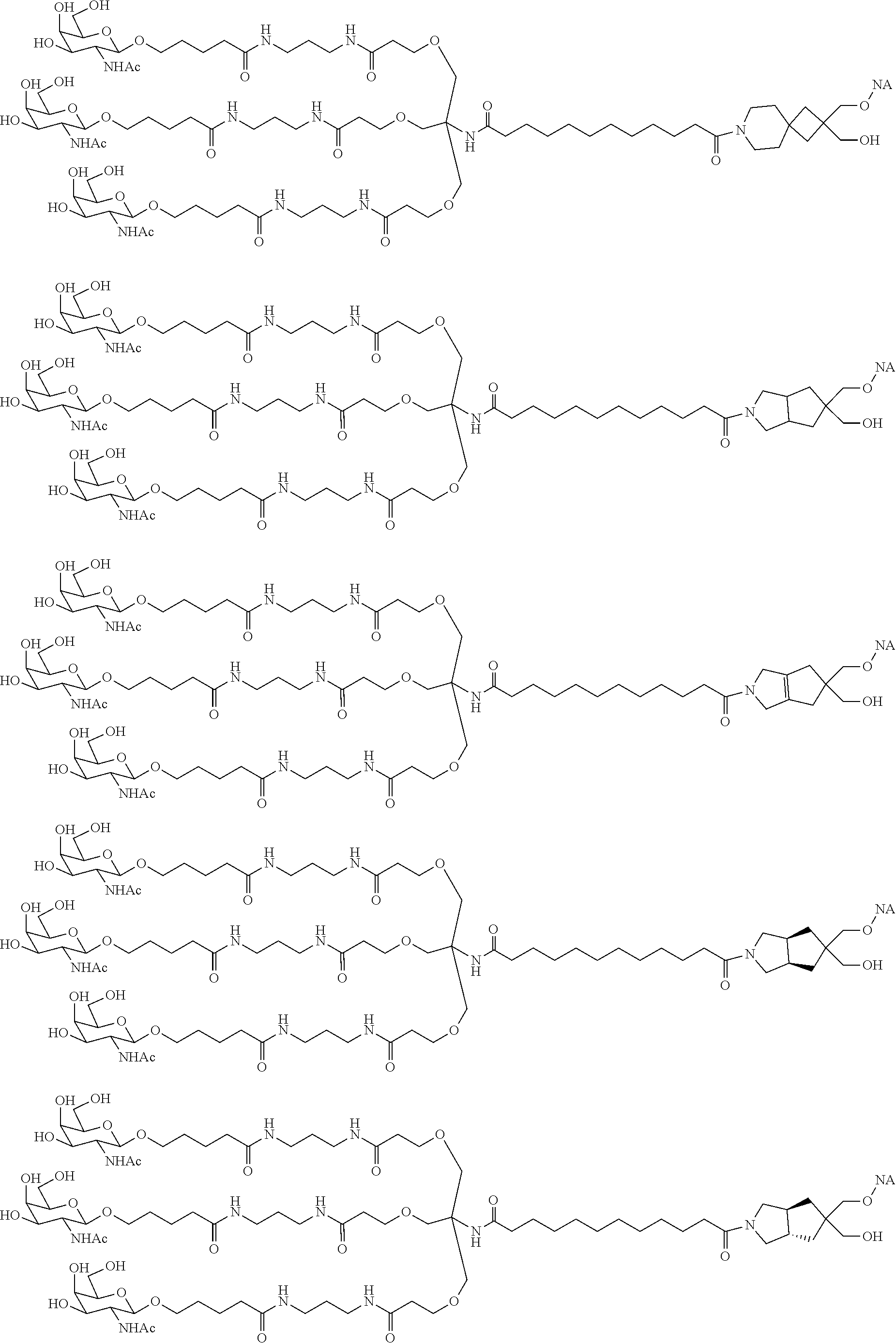 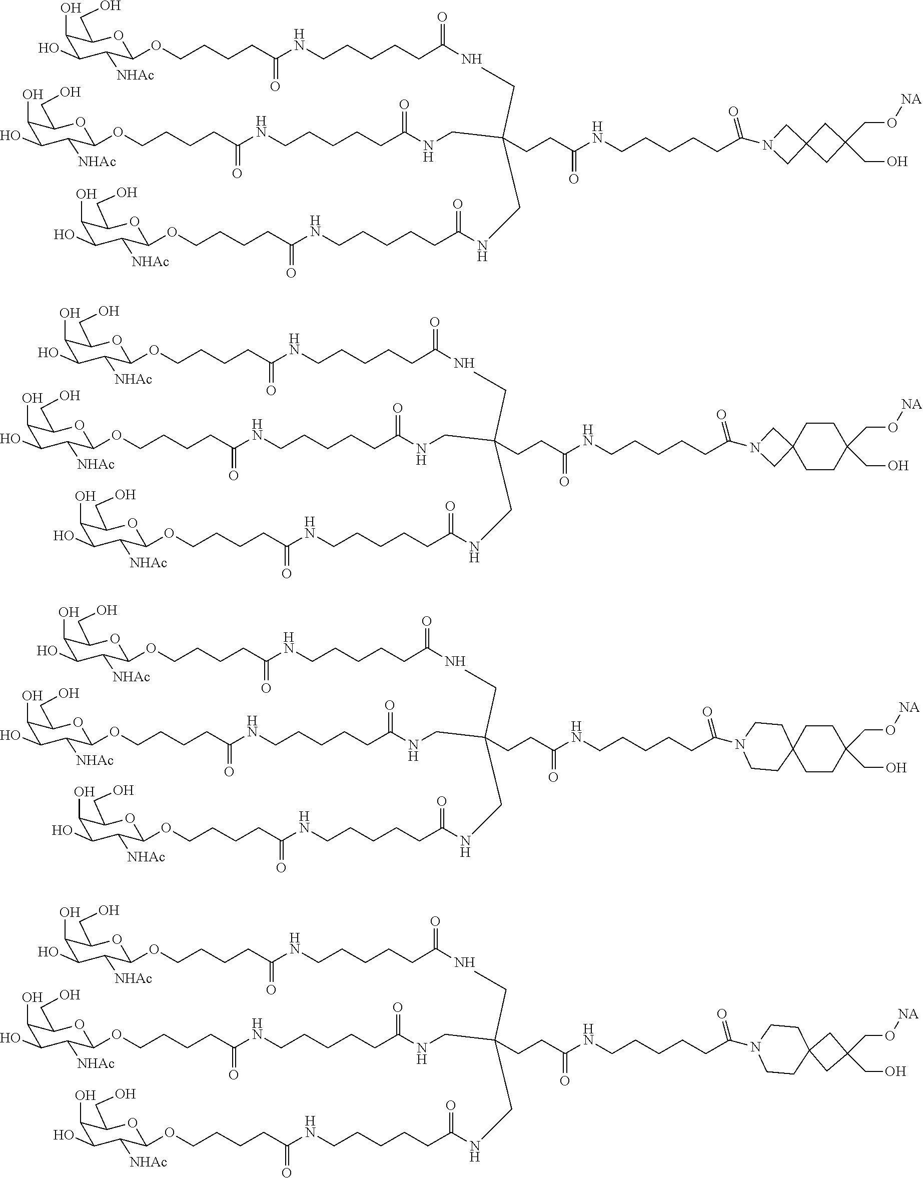 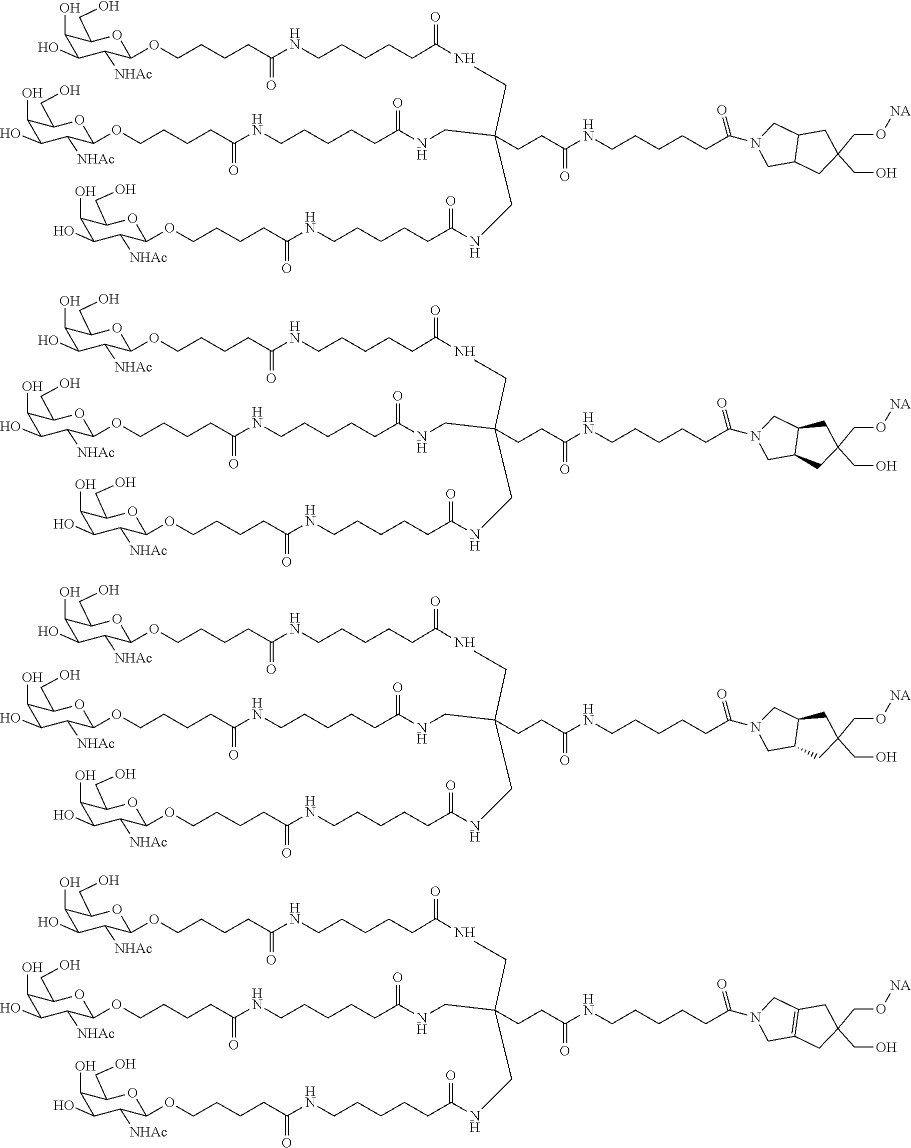 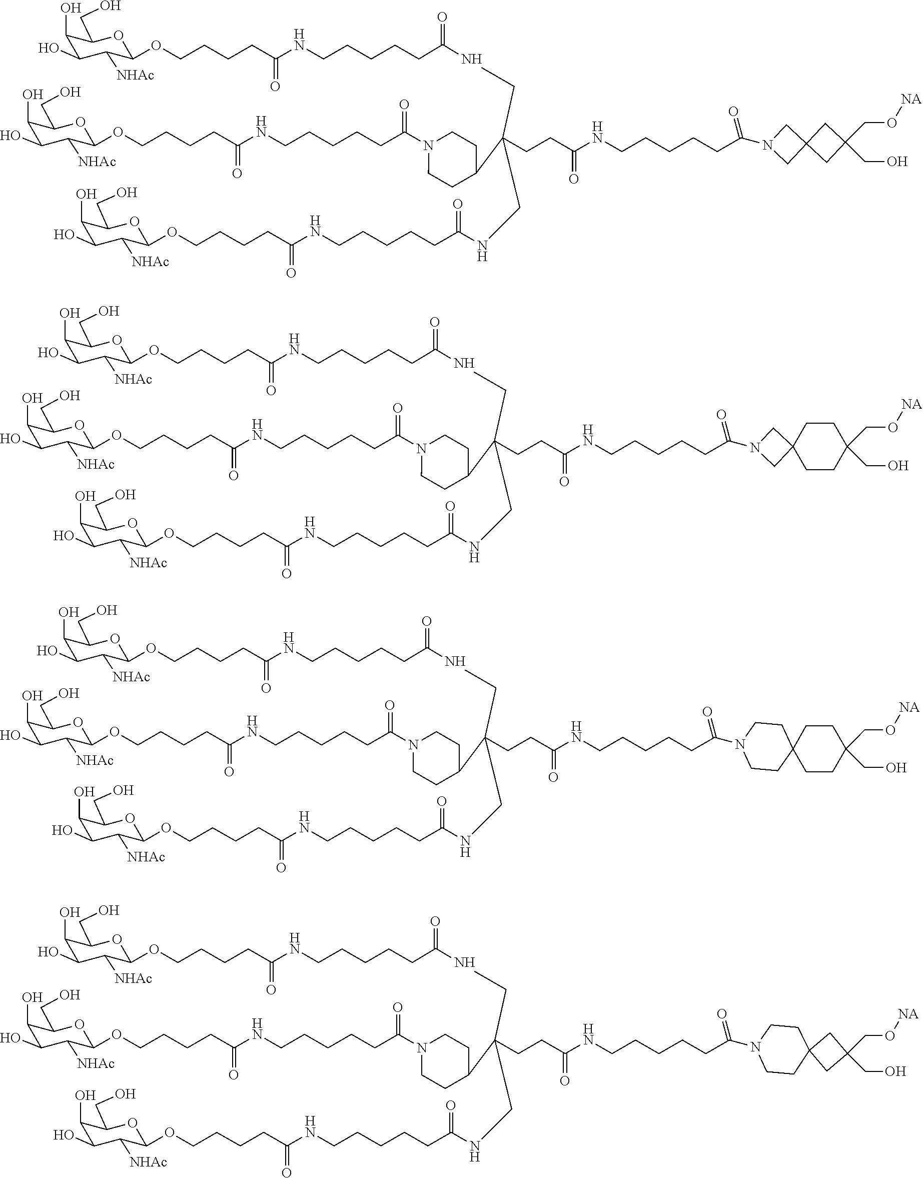 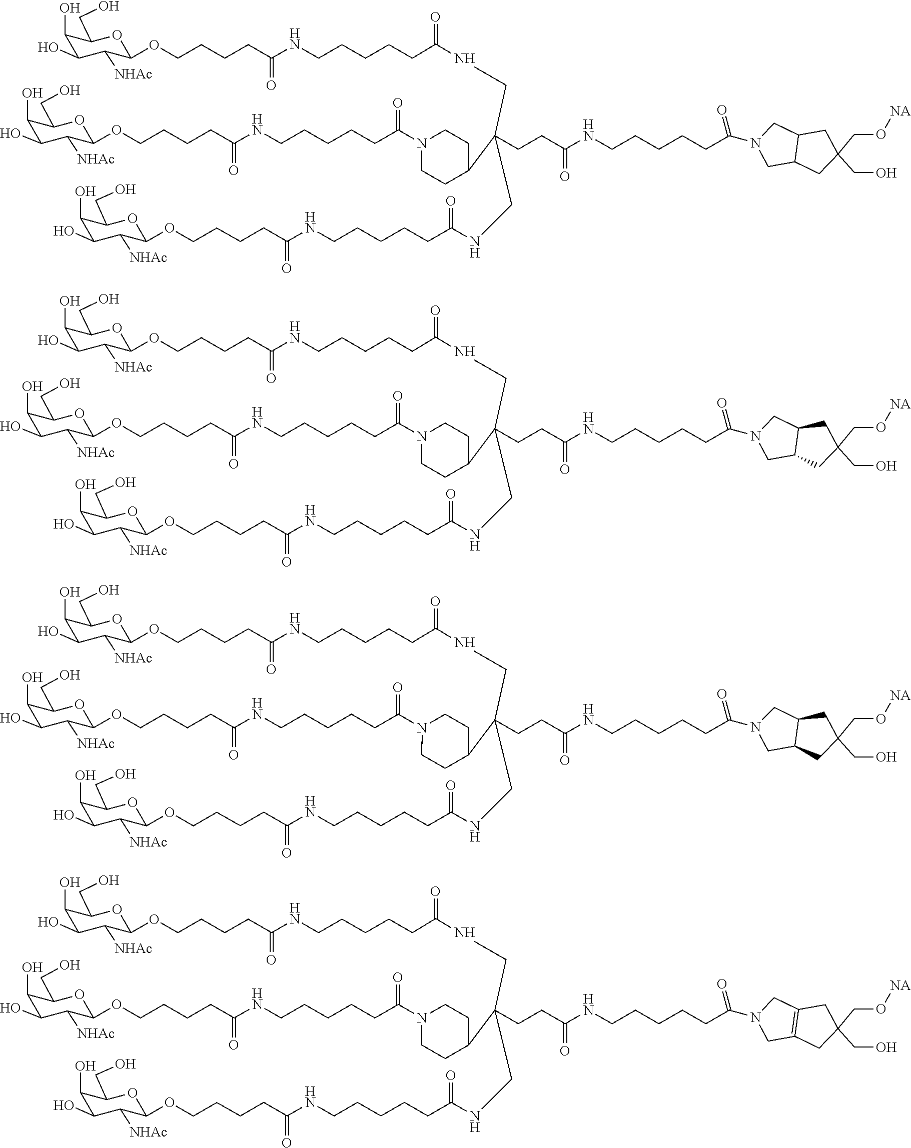 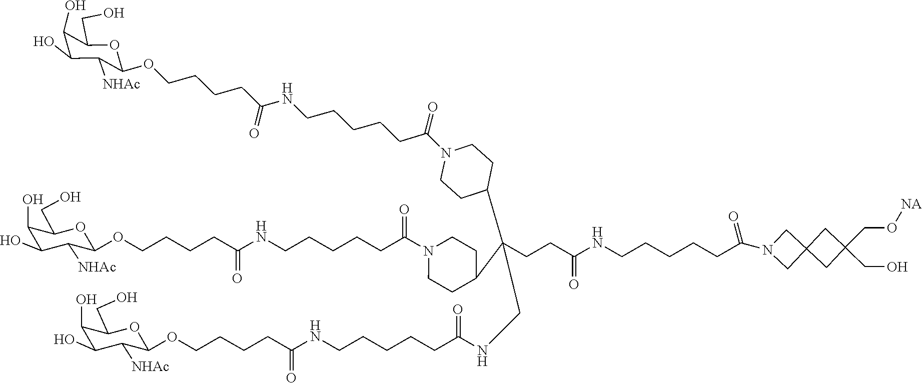 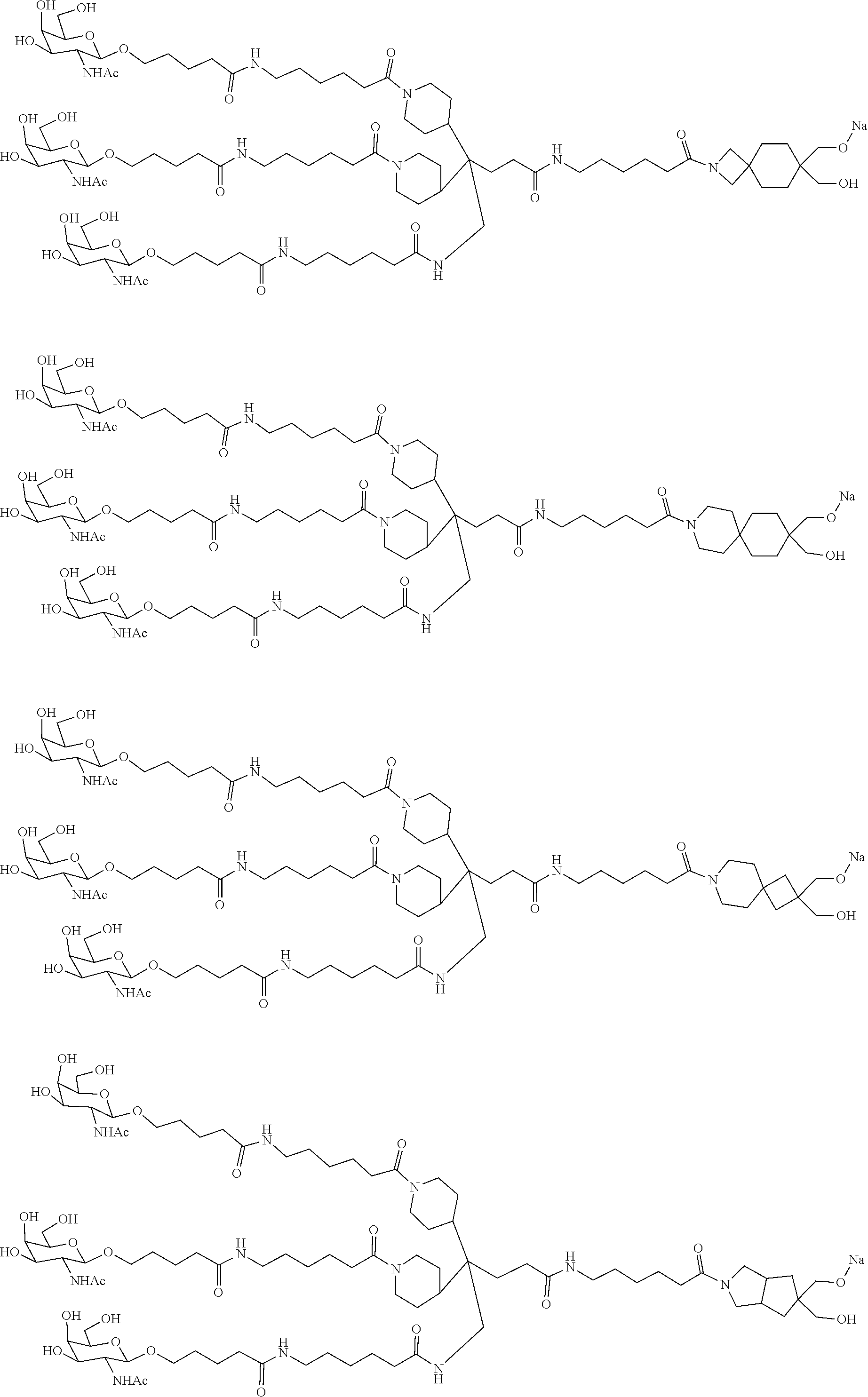 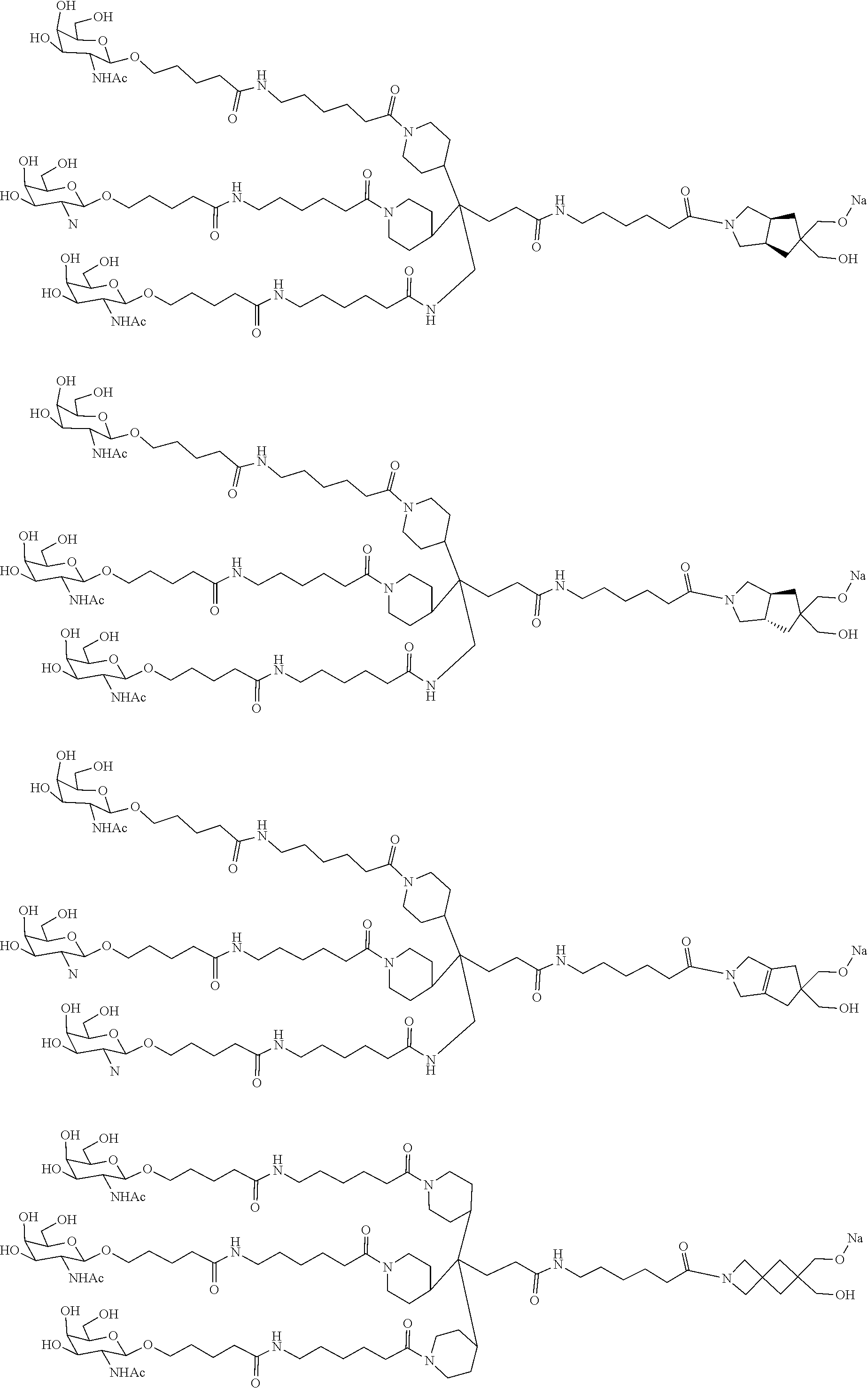 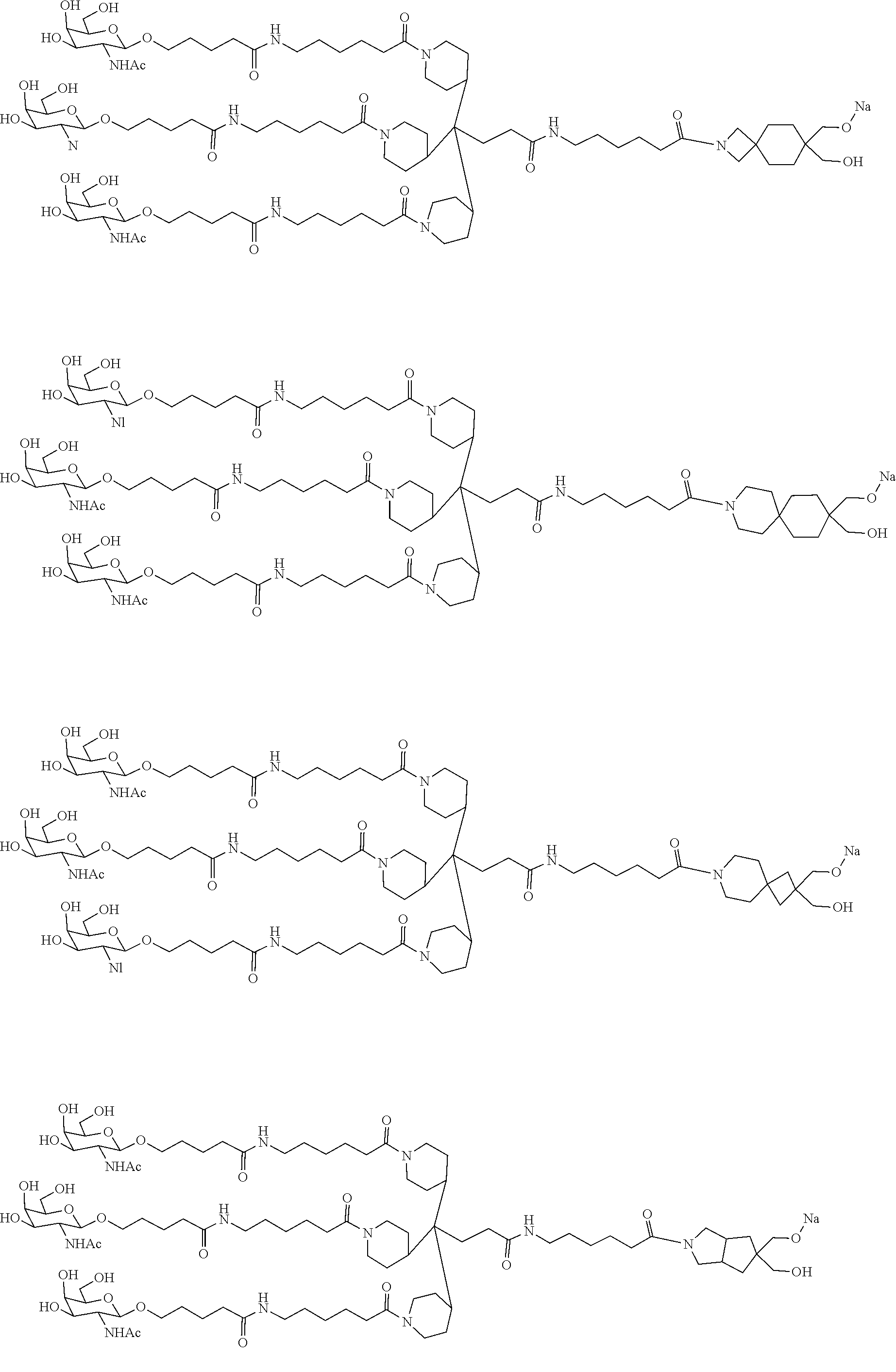 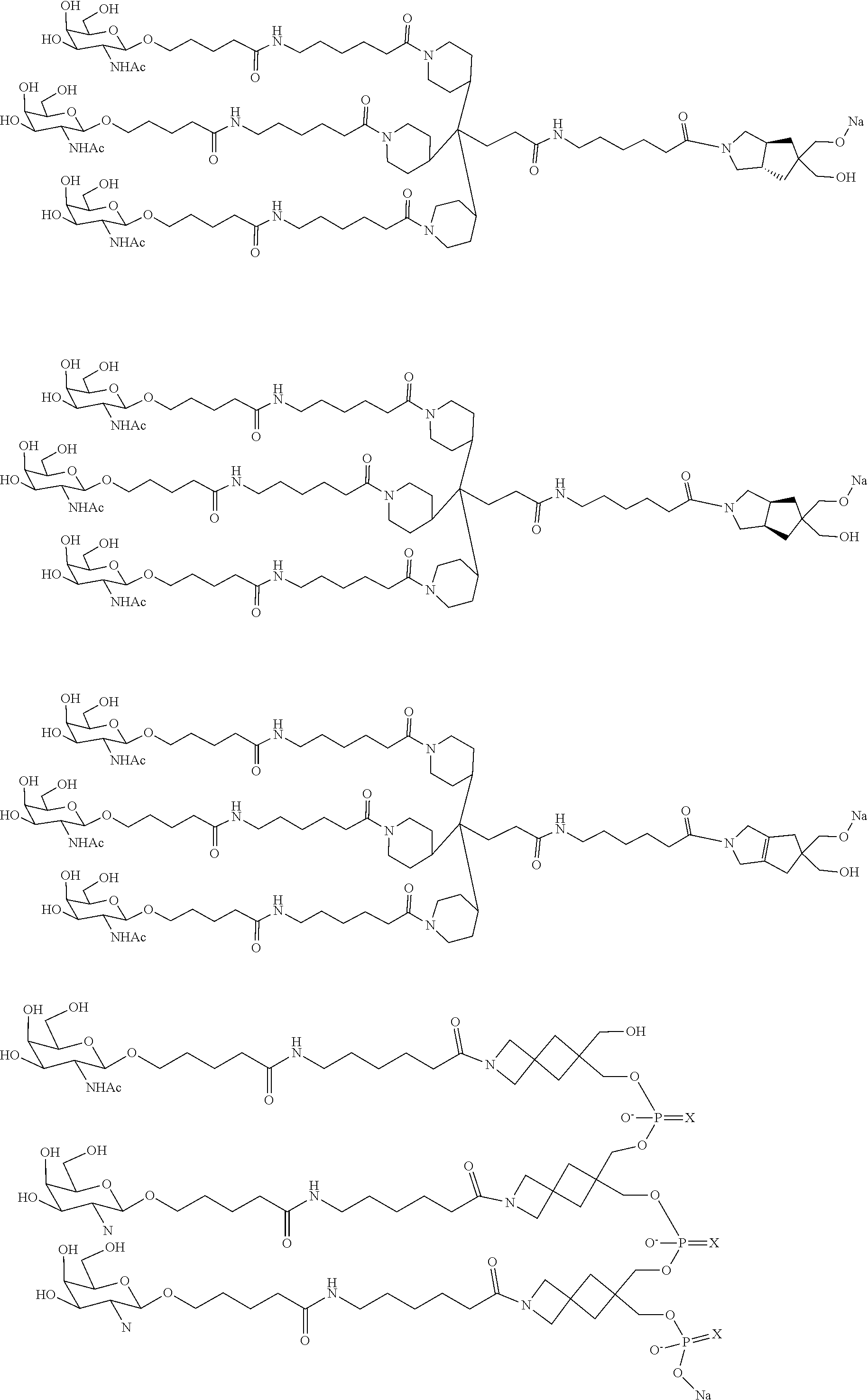 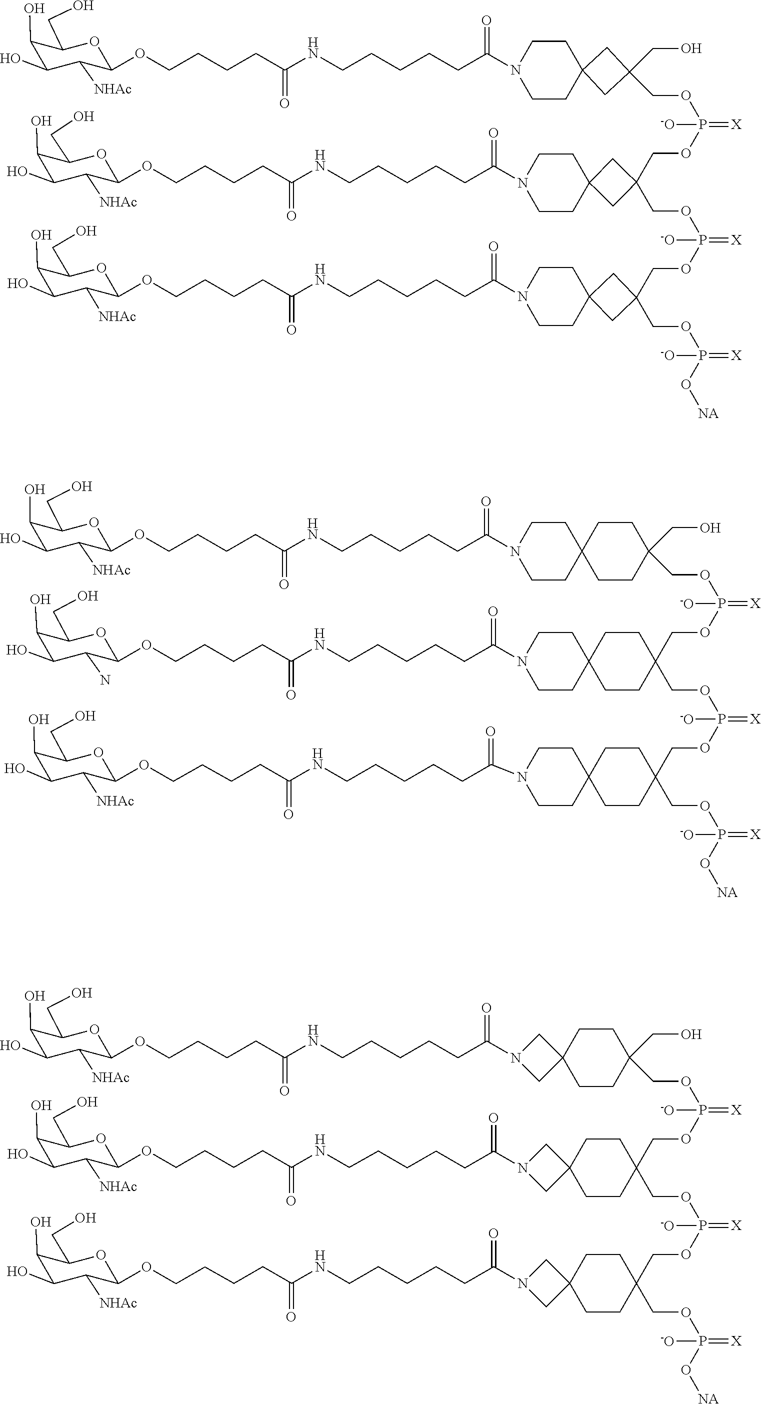 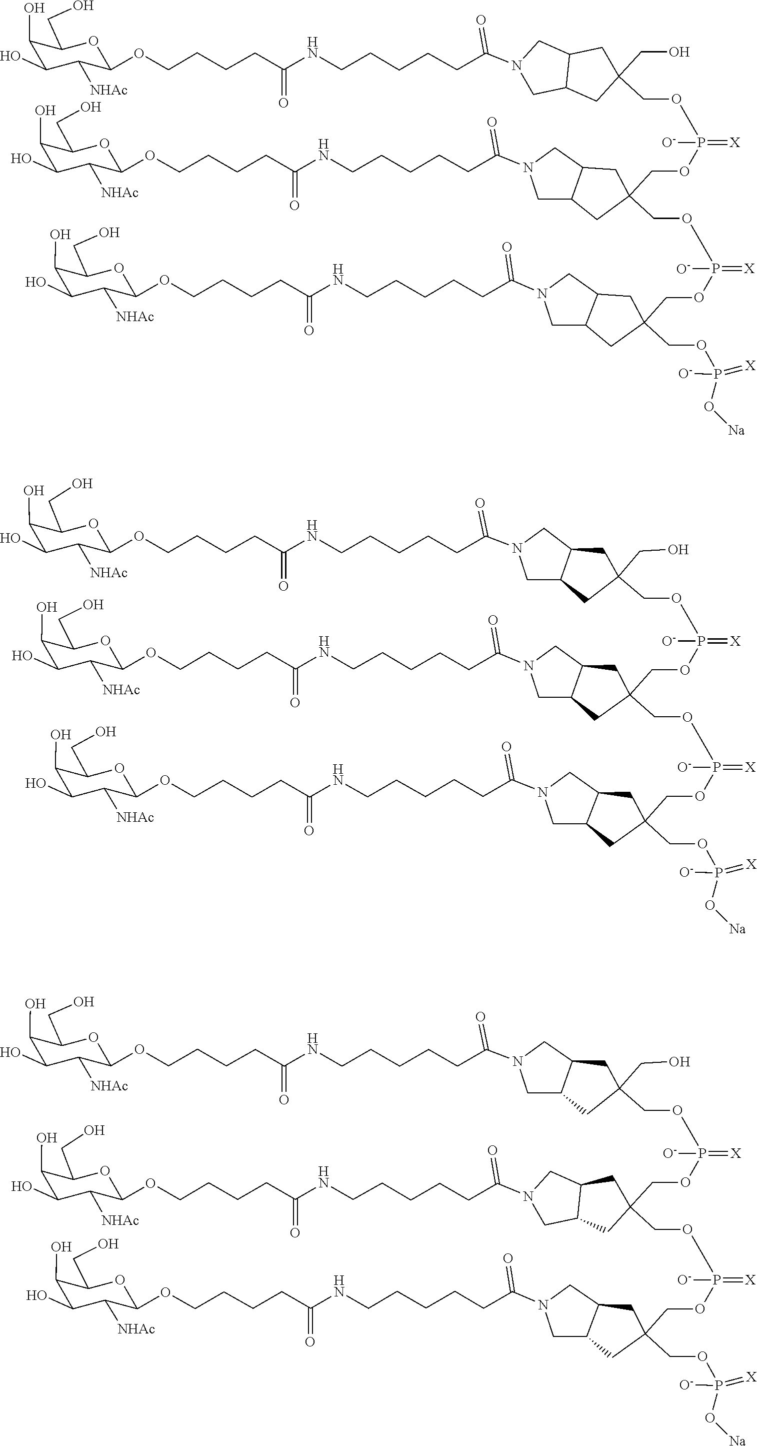 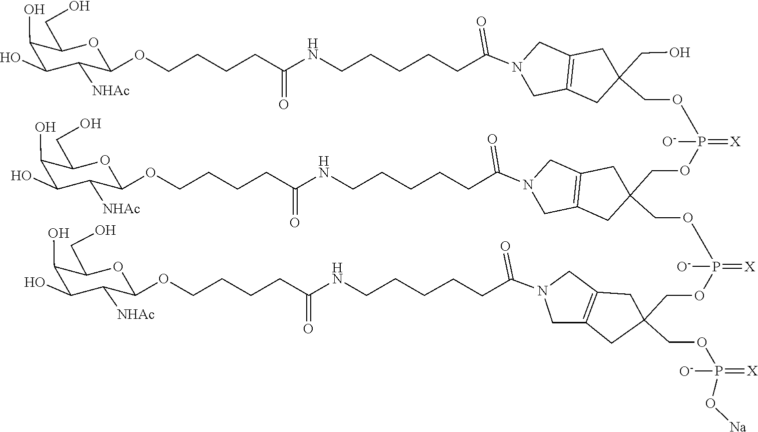
NA is oligonucleotide;
X is O or S.

16. The therapeutic compound of claim 14, wherein the oligonucleotide is antisense oligonucleotide; preferably, the oligonucleotide is siRNA.

17. A pharmaceutical composition comprising the therapeutic compound of claim 14 or pharmaceutically acceptable salt thereof, and a pharmaceutically acceptable carrier, diluent or excipients.

18. A method for treating a disease or disorder that would benefit from administration of the therapeutic compound of claim 14 or pharmaceutically acceptable salt thereof.

19. A method of synthesizing the therapeutic compound of claim 14, comprising:
providing a targeting ligand of claim 1, and conjugating the targeting ligand to an oligonucleotide; preferably, the targeting ligand conjugate to the 3′ end or the 5′ end of oligonucleotide; more preferably, the oligonucleotide is siRNA.

20. A compound of formula (V-a), (V-b), (V-c), (VI-a), (VI-b), or (VI-c), or tautomer, pharmaceutically acceptable salt thereof: 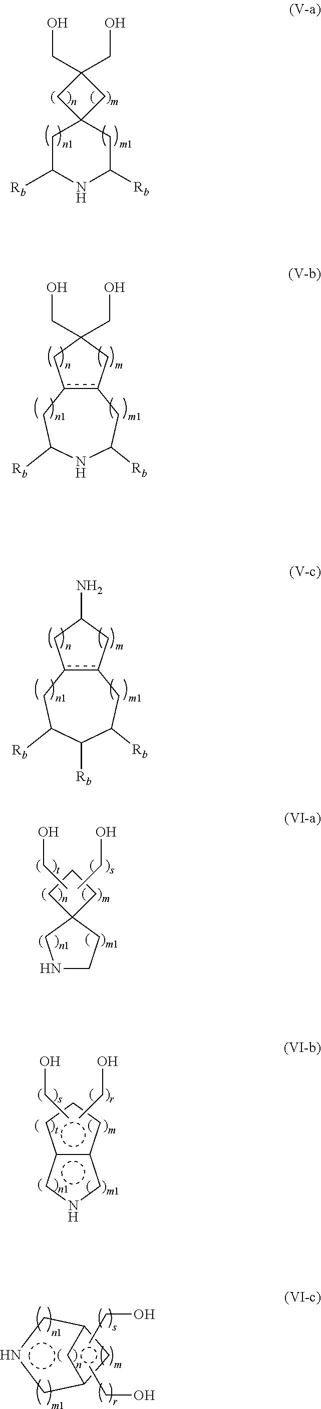
(i) in the formula (V-a), (V-b), (V-c): [symbol]
is a single or double bond;
R_{b} is independently selected from the group of hydrogen and oxo;
each of m and n is independently selected from 1, 2, 3; 4 and 5 each of m1 and n1 is each independently selected from 0, 1, 2, 3 and 4;
(ii) in the formula (VI-a), (VI-b), or (VI-c),
m is independently selected from 0, 1, 2, 3; 4 and 5;
n is independently selected from 1, 2, 3; 4 and 5;
each of r, s and t is independently selected from 0, 1 and 2;
in the formula (VI-a), m1 and n1 is each independently selected from 0, 1, 2, 3 and 4,
and in the formula (VI-b) or (VI-c), m1 is 1, n1 is 0, 2, 3 or 4.

21. (canceled)

22. A compound of claim 20 having the following structure: 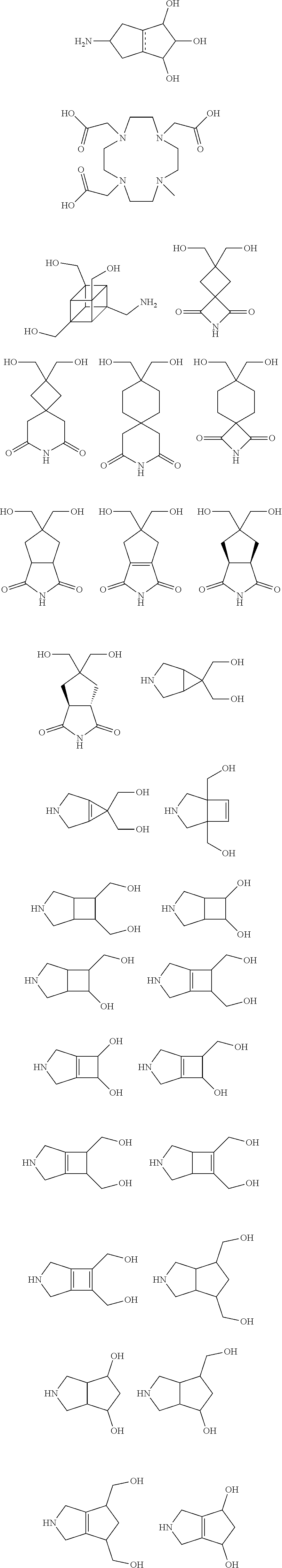 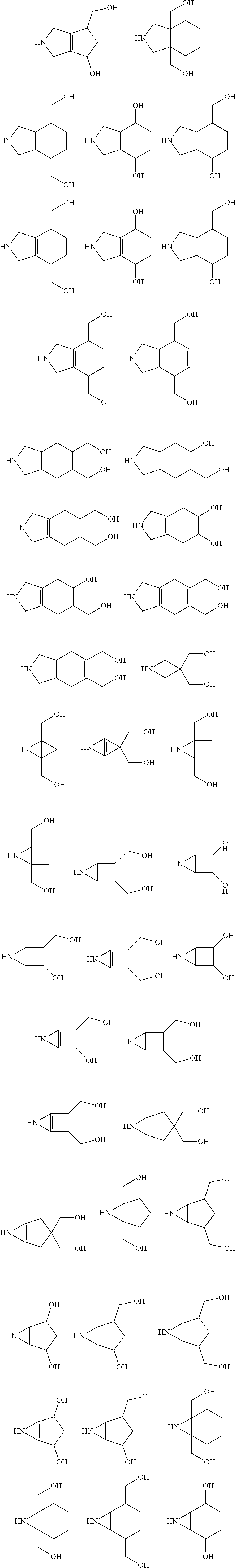 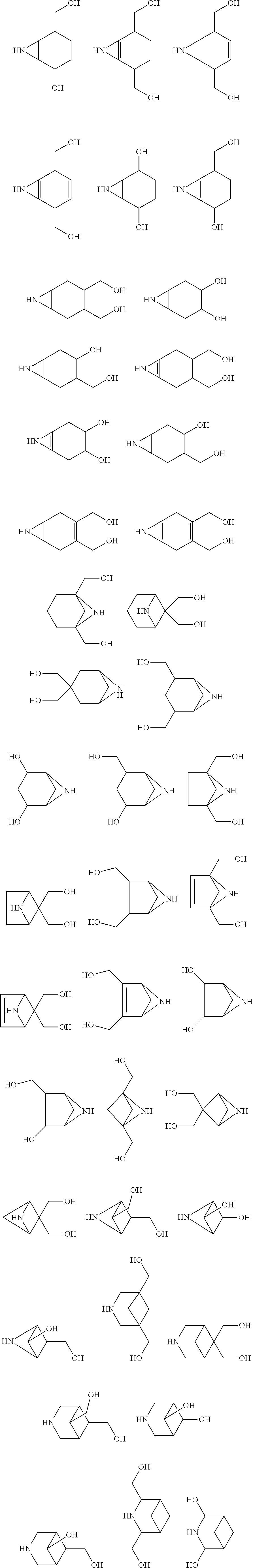 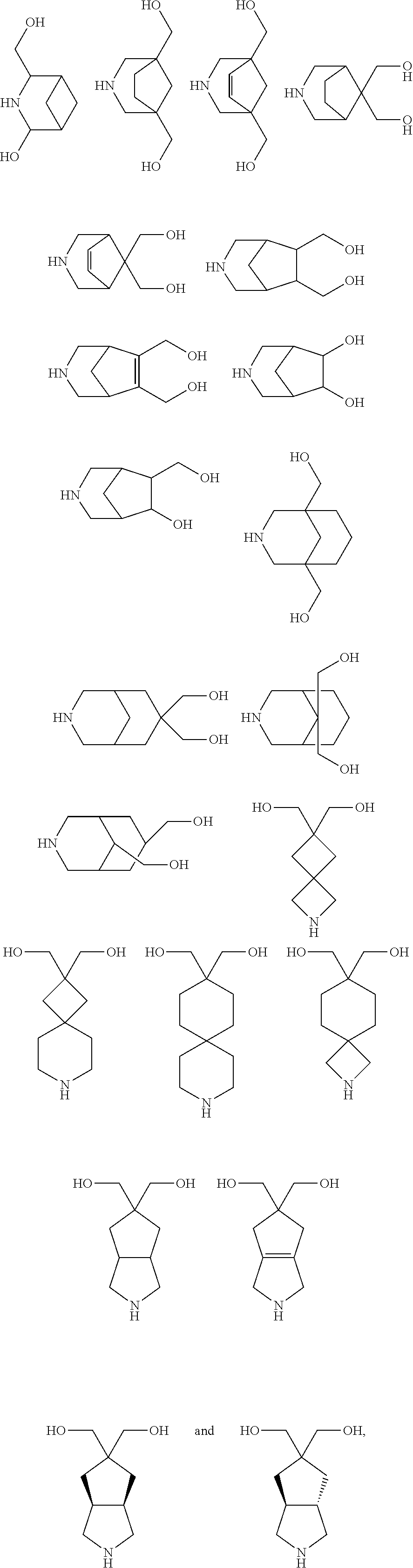
or tautomer, pharmaceutically acceptable salt thereof.
